(19) United States
(12) Patent Application Publication
(10) Pub. No.: US 2026/0265262 A1
(43) Pub. Date: Sep. 10, 2026

(54) CDK2 INHIBITORS AND METHODS OF USING THE SAME

(71) Applicant: Cedilla Therapeutics, Inc., Foxboro, MA (US)

(72) Inventors: Louise Clare KIRMAN, Foxboro, MA (US); Carl Eric SCHWARTZ, Foxboro, MA (US); Thomas P. BLAISDELL, Foxboro, MA (US)

(21) Appl. No.: 18/995,330

(22) PCT Filed: Jul. 28, 2023
(86) PCT No.: PCT/US2023/071258

§ 371 Date: Jan. 16, 2025

Related U.S. Application Data
(60) Provisional application No. 63/393,066, filed on Jul. 28, 2022.

(51) Int. Cl. C07D 487/10; A61K 31/4155; A61K 31/422; A61K 31/427; A61K 31/438; A61K 31/454; A61K 31/4545; A61K 31/497; A61K 31/675; A61P 35/00; C07D 519/00; C07F 9/6561

(52) U.S. Cl. CPC C07D 487/10; A61K 31/4155; A61K 31/422; A61K 31/427; A61K 31/438; A61K 31/454; A61K 31/4545; A61K 31/497; A61K 31/675; A61P 35/00; C07D 519/00; C07F 9/6561

(57) ABSTRACT

The present disclosure provides compounds, compositions thereof, and methods of using the same for the inhibition of CDK2, and the treatment of CDK2 related diseases and disorders.

### CROSS REFERENCE TO RELATED APPLICATIONS

This application claims the benefit of priority to U.S. Provisional Application No. 63/393,066, filed Jul. 28, 2022, the entire contents of which is herein incorporated by reference.

### FIELD

The present disclosure relates generally to Cyclin-dependent kinase 2 (CDK2) inhibiting chemical compounds and uses thereof in the inhibition of the activity of CDK2. The disclosure also provides pharmaceutically acceptable compositions comprising compounds disclosed herein and methods of using said compounds and compositions in the treatment of various disorders related to CDK2 activity.

### BACKGROUND

Cell cycle dysregulation, including uncontrolled cell growth, impaired cell differentiation and abnormal apoptosis have been shown to be caused by over activity of Cyclin-dependent kinases (CDKs). CDKs are important serine/threonine protein kinases that become active when combined with a specific cyclin partner. There are various subtypes of CDKs, each having a different role during the cell cycle, with varying levels of activity during each of the phases. CDK1, CDK2, CDK4 and CDK6 have been found to be specifically important subtypes, where over activity of one or more of these subtypes may lead to dysregulation of the cell cycle and the development of a variety of cancers. The S phase of the cell cycle is responsible for DNA replication and is the phase where aberrant DNA replication may occur. The CDK2/cyclin E complex is required for the cell cycle transition from the G1 phase to the S phase and the CDK2/cyclin A complex is required for the cell cycle transition from the S phase to the G2 phase. Therefore, selective inhibition of the CDK2/cyclin E and/or CDK2/cyclin A complexes can prevent aberrant DNA replication and can be used to treat certain cancers.

Accordingly, there is a need for the development of compounds capable of inhibiting the activity of CDK2/cyclin complexes, and pharmaceutical compositions thereof, for the prevention, and treatment of CDK2 related diseases or disorders.

### SUMMARY

The present disclosure is based at least in part on the identification of compounds that bind and inhibit Cyclin-dependent kinase 2 (CDK2) and/or CDK2/cyclin complexes and methods of using the same to treat diseases associated with CDK2 activity. Disclosed herein is a compound according to Formula IA or a pharmaceutically acceptable salt thereof:

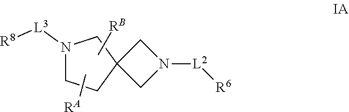

wherein each variable is as defined and described herein.

Compounds of the present disclosure, and pharmaceutically acceptable compositions thereof, are useful for treating a variety of diseases, disorders or conditions, associated with CDK2 activity. Such diseases, disorders, or conditions include those described herein.

### DETAILED DESCRIPTION OF CERTAIN EMBODIMENTS

### 1. General Description of Compounds of the Disclosure

The present disclosure provides compounds capable of inhibiting Cyclin-dependent kinase 2 (CDK2) and/or CDK2/cyclin complexes.

In some embodiments, the inhibitors of CDK2 include compounds of Formula IA:

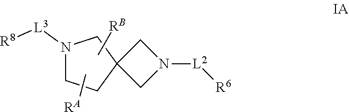

or a pharmaceutically acceptable salt thereof, wherein:
R^{A} is

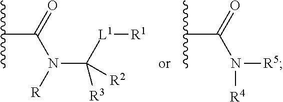

R^{B} is hydrogen, an optionally substituted C₁₋₆ aliphatic group, —OR, —NR₂ or a halogen;
L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
R² is hydrogen, an optionally substituted C₁₋₆ aliphatic group, —C₁₋₆ alkylene-OR, —C₁₋₃ alkylene-O—C₁₋₃ alkylene-R, —C(O)OR, —C(O)NR₂, —S(O)₂R, —S(O)₂NR₂, —S(O)R, —P(O)R₂, —C(O)NRS(O)₂R, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R³ is hydrogen; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R⁵ is hydrogen; or
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
when there are two R groups on the same nitrogen they are taken together with their intervening atoms to form a cyclic group selected from a 4-7 membered saturated, partially unsaturated, or heteroaryl ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms independently selected from nitrogen, oxygen, and sulfur) wherein the cyclic group is optionally substituted with one or more instances of R⁹;
wherein the compound is not Compound X, wherein Compound X is defined herein.

Overexpression of CDK2 is associated with abnormal regulation of the cell-cycle. The cyclin E/CDK2 complex plays an important role in regulation of the G1/S transition, histone biosynthesis and centrosome duplication. Progressive phosphorylation of retinoblastoma (Rb) by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al., Nat. Rev. Drug. Discov. 2015; 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Mol. Cancer Ther. (2012) 11:1488-99; Herrera-Abreu et al., Cancer Res. (2016) 76: 2301-2313). Cyclin E amplification also reportedly contributes to trastuzumab resistance in HER2+ breast cancer. (Scaltriti et al., Proc Natl Acad Sci. (2011) 108: 3761-6). Cyclin E overexpression has also been reported to play a role in basal-like and triple negative breast cancer (TNBC), as well as inflammatory breast cancer. (Elsawaf & Sinn, Breast Care (2011) 6:273-278; Alexander et al., Oncotarget (2017) 8: 14897-14911).

Amplification or overexpression of cyclin E1 (CCNE1) is also associated with poor outcomes in ovarian, gastric, endometrial and other cancers. (Nakayama et al., Gene amplification CCNE1 is related to poor survival and potential therapeutic target in ovarian cancer, Cancer (2010) 116: 2621-34; Etemadmoghadam et al., Clin Cancer Res (2013) 19: 5960-71; Au-Yeung et al., Clin. Cancer Res. (2017) 23:1862-1874; Ayhan et al., Modern Pathology (2017) 30: 297-303; Ooi et al., Hum Pathol. (2017) 61: 58-67; Noske et al., Oncotarget (2017) 8: 14794-14805).

There remains a need in the art for CDK inhibitors, especially selective CDK2 inhibitors, which may be useful for the treatment of cancer or other proliferative diseases or conditions. In particular, CDK2 inhibitors may be useful in treating CCNE1 or CCNE2 amplified tumors.

### 2. Compounds and Definitions

Compounds of this present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. “Bicyclic” may refer to a “bridged bicyclic” or “spirocyclic” ring. As used herein, “bridged bicyclic” rings are to be understood to be a subset of, and falling within the scope of, “bicyclic ring”. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

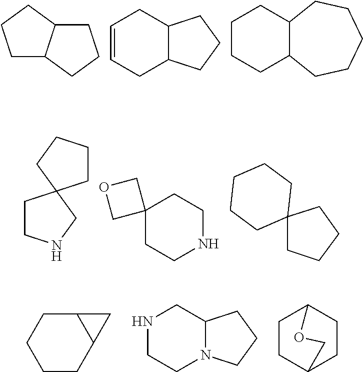

Exemplary bridged bicyclics, contemplated as falling under the scope of a “bicycle” or “bicyclic ring” include:

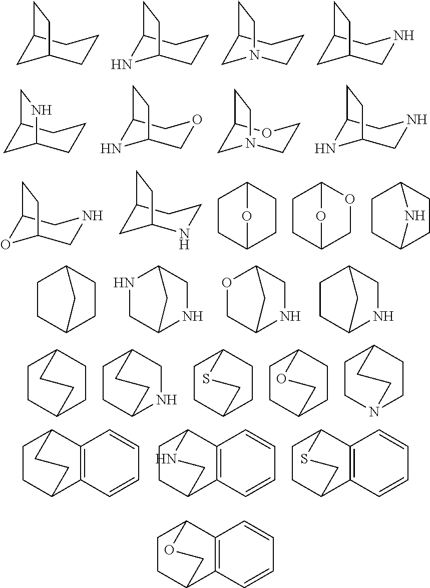

The term “Compound X” refers to 6-(1-benzyl-1H-pyrazole-4-carbonyl)-N-(3-(benzyloxy)-1-(methylamino)-1-oxobutan-2-yl)-2-(2,2-dimethylcyclopropane-1-carbonyl)-2,6-diazaspiro[3.4]octane-8-carboxamide. Compound X may also be depicted as

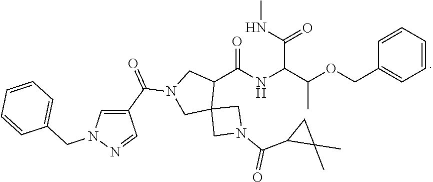 [SMILES: CNC(=O)C(NC(=O)C1CN(C(=O)c2cnn(Cc3ccccc3)c2)CC12CN(C(=O)C1CC1(C)C)C2)C(C)OCc1ccccc1]

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted.

Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₆R^{o}; —(CH₂)₀₋₆OR^{o}; —O(CH₂)₀₋₆R^{o}, —O—(CH₂)₀₋₆C(O)OR^{o}; —(CH₂)O—CH(OR^{o})₂; —(CH₂)₀₋₆SR^{o}; —(CH₂)₀₋₆Ph, which Ph may be substituted with R^{o}; —(CH₂)₀₋₄₆O(CH₂)₀₋₁Ph which Ph may be substituted with R^{o}; —CH═CHPh, which Ph may be substituted with R^{o}; —(CH₂)₀₋₆O(CH₂)₀₋₁-pyridyl which pyridyl may be substituted with R^{o}; —NO₂; —CN; —N₃; —(CH₂)₀₋₆N(R^{o})₂; —(CH₂)₀₋₆N(R^{o})C(O)R^{o}; —N(R^{o})C(S)R^{o}; —(CH₂)₀₋₆N(R^{o})C(O)NR^{o}₂; —N(R^{o})C(S)NR^{o}₂, —(CH₂)₀₋₆N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₆C(O)R^{o}; —C(S)R^{o}; —(CH₂)₀₋₆C(O)OR^{o}; —(CH₂)₀₋₆C(O)SR^{o}; —(CH₂)₀₋₆C(O)OSiR^{o}₃; —(CH₂)₀₋₆OC(O)R^{o}; —OC(O)(CH₂)₀₋₆SR^{o}, —(CH₂)₀₋₆SC(O)R^{o}; —(CH₂)₀₋₆C(O)NR^{o}₂; —C(S)NR^{o}₂; —C(S)SR^{o}; —SC(S)SR^{o}, —(CH₂)₀₋₆OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₆SSR^{o}; —(CH₂)₀₋₆S(O)₂R^{o}; —(CH₂)₀₋₆S(O)₂OR^{o}; —(CH₂)₀₋₆—OS(O)₂R^{o}; —S(O)₂NR^{o}₂; —(CH₂)₀₋₆S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —P(O)(OR^{o})₂; —OP(O)(R^{o})OR^{o}; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5- to 6-membered heteroaryl ring), —CH₂-(5- to 6-membered saturated or partially unsaturated monocyclic heterocyclic ring [having 1-2 heteroatoms independently selected from nitrogen, oxygen, and sulfur]), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{o}, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below. An “optionally substituted” group may be substituted with one or more (i.e., from 1 to 6) substituents selected from the aforementioned monovalent substituents and one or more (i.e., from 1 to 6) suitable divalent substituents of ═O, ═S, ═NNR^{o}₂, ═NNHC(O)R^{o}, ═NNHC(O)OR^{o}, ═NNHS(O)₂R^{o}, ═NR^{o}, ═NOR^{o}, —O(C(R^{o}₂))₂₋₃O—, —(C(R^{o})₂)₂₋₆—, and —S(C(R^{o}₂))₂₋₃S—, wherein each of the one or more substituents is independently selected from said monovalent substituents and divalent substituents, and wherein said divalent substituents may be on a saturated carbon of an “optionally substituted” group.

Suitable monovalent substituents on R^{o} (or the ring formed by taking two independent occurrences of R^{o} together with their intervening atoms), wherein R^{o} may be substituted with one or more instances of said monovalent substituents (i.e., from 1 to 6) and suitable divalent substituents described at the end of this paragraph, and said monovalent substituents are each independently halogen, —(CH₂)₀₋₂R^{•}, -(haloR^{•}), —(CH₂)₀₋₂OH, —(CH₂)₀₋₂OR*, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur). Suitable divalent substituents on a saturated carbon atom of R^{o} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(Rt)S(O)₂R^{†}; wherein each R^{†} is independently hydrogen, C₁₋₆ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of RT, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

As used herein, the term “provided compound” or “compound of the present disclosure” refers to any genus, subgenus, and/or species set forth herein.

“One or more instances” or “one or more” as referencing substitutions, as used herein, refers to, for example, 1, 2, 3, 4, 5, 6, 7, etc. instances of substitution of functional groups, which may each be independently selected, on a chemical moiety to which “one or more” instances of substitution refers. It is to be understood that any “optionally substituted” moiety, may be substituted with “one or more” optional substituents each independently selected from those optional substituents as described herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

As used herein, the term “inhibitor” is defined as a compound that binds to and/or inhibits CDK2 with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM, when measured in an appropriate assay.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily or degratorily active metabolite or residue thereof.

As used herein, the term “inhibitorily active metabolite or residue thereof” means that a metabolite or residue thereof is also an inhibitor of a CDK2 protein, or a mutant thereof.

### 3. Description of Exemplary Embodiments

In certain embodiments, the present disclosure provides inhibitors of CDK2 activity.

In some embodiments, the inhibitors of CDK2 include compounds of Formula IA:

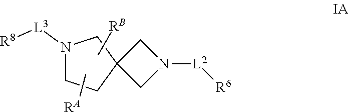

or a pharmaceutically acceptable salt thereof, wherein:
R^{A} is

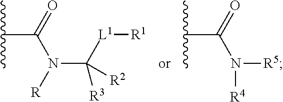

R^{B} is hydrogen, an optionally substituted C₁₋₆ aliphatic group, —OR, —NR₂ or a halogen;
L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
R² is hydrogen, an optionally substituted C₁₋₆ aliphatic group, —C₁₋₆ alkylene-OR, —C₁₋₃ alkylene-O—C₁₋₃ alkylene-R, —C(O)OR, —C(O)NR₂, —S(O)₂R, —S(O)₂NR₂, —S(O)R, —P(O)R₂, —C(O)NRS(O)₂R, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R³ is hydrogen; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R⁵ is hydrogen; or
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
when there are two R groups on the same nitrogen they are taken together with their intervening atoms to form a cyclic group selected from a 4-7 membered saturated, partially unsaturated, or heteroaryl ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹; and
wherein the compound is not Compound X, wherein Compound X is defined herein.

In some embodiments, the inhibitors of CDK2 include compounds of Formula I:

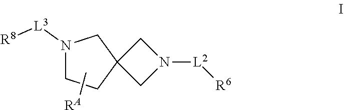

or a pharmaceutically acceptable salt thereof, wherein:
R^{A} is

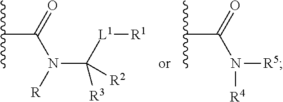

L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
R² is an optionally substituted C₁₋₆ aliphatic group, —C₁₋₆ alkylene-OR, —C(O)OR, —C(O)NR₂, —C(O)NRS(O)₂R, or an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen;
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L¹ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
when there are two R groups on the same nitrogen they are taken together with their intervening atoms to form a cyclic group selected from a 4-7 membered saturated, partially unsaturated, or heteroaryl ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹; and
wherein the compound is not Compound X, wherein Compound X is defined herein.

As defined generally above, R^{A} is

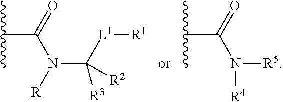

In some embodiments, R^{A} is

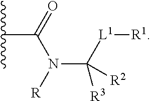

In some embodiments, R^{A} is

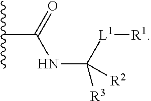

In some embodiments, R^{A} is

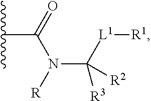

wherein the R group shown is an optionally substituted C₁₋₆ aliphatic group. In some embodiments, R^{A} is

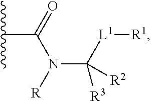

wherein the R group shown is an optionally substituted methyl group. In some embodiments, R^{A} is

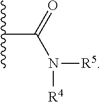

In some embodiments, R^{A} is

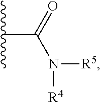

wherein R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur). In some embodiments, R^{A} is selected from those depicted in the compounds of Table 1, below.

As defined generally above, R^{B} is hydrogen, an optionally substituted C₁₋₆ aliphatic group, —OR, —NR₂ or a halogen. In some embodiments, R^{B} is hydrogen. In some embodiments, R^{B} is an optionally substituted C₁₋₆ aliphatic group. In some embodiments, R^{B} is —OR. In some embodiments, R^{B} is —NR₂. In some embodiments, R^{B} is a halogen. In some embodiments, R^{B} is a methyl group. In some embodiments, R^{B} is a fluoro group. In some embodiments, R^{B} is selected from those depicted in the compounds of Table 1, below.

As defined generally above, L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L¹ is a covalent bond. In some embodiments, L¹ is a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O— or —NRC(O)NR—. In some embodiments, L¹ is a saturated or unsaturated, straight or branched, optionally substituted bivalent C₁₋₄ hydrocarbon chain, wherein 0-2 methylene units of L are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is a saturated or unsaturated, straight or branched, optionally substituted bivalent C₁₋₄ hydrocarbon chain. In some embodiments, L¹ is a saturated or unsaturated, straight or branched, optionally substituted bivalent C₁₋₄ hydrocarbon chain, wherein 1 or 2 methylene units of L¹ are replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is a saturated, straight or branched, optionally substituted bivalent C₁₋₄ hydrocarbon chain. In some embodiments, L¹ is a partially unsaturated, straight or branched, optionally substituted bivalent C₁₋₄ hydrocarbon chain. In some embodiments, L¹ is a saturated, straight, optionally substituted bivalent C₁₋₄ hydrocarbon chain, wherein 1-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—. In some embodiments, L¹ is an optionally substituted straight or branched C₁₋₄ alkylene chain, wherein 1-2 methylene units of L¹ are independently replaced by —O—, —NR—, —S—, —C(O)O—, —C(O)—, —S(O)₂—, or —NRC(O)—. In some embodiments, L¹ is an optionally substituted straight or branched C₁₋₄ alkylene chain, wherein 1-2 methylene units of L¹ are independently replaced by —O—, —NR—, —C(O)O—, —C(O)—, or —NRC(O)—. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is an optionally substituted straight or branched C₁₋₄ alkylene chain, wherein 1-2 methylene units of L¹ are independently replaced by —O—. In some embodiments, L¹ is an optionally substituted straight or branched C₁₋₄ alkylene chain, wherein 1-2 methylene units of L¹ are independently replaced by —S—. In some embodiments, L¹ is an optionally substituted straight or branched C₁₋₄ alkylene chain, wherein 1-2 methylene units of L¹ are independently replaced by —S(O)₂—. In some embodiments, L¹ is an optionally substituted straight or branched C₁₋₄ alkylene chain, wherein 1-2 methylene units of L¹ are independently replaced by —NR—. In some embodiments, L¹ is an optionally substituted straight or branched C₁₋₄ alkylene chain, wherein 1-2 methylene units of L¹ are independently replaced by —C(O)O—. In some embodiments, L¹ is an optionally substituted straight or branched C₁₋₄ alkylene chain, wherein 1-2 methylene units of L¹ are independently replaced by —NRC(O)—. In some embodiments, L¹ is an unsubstituted straight chain C₁₋₄ alkynylene.

In some embodiments, L¹ is a covalent bond,

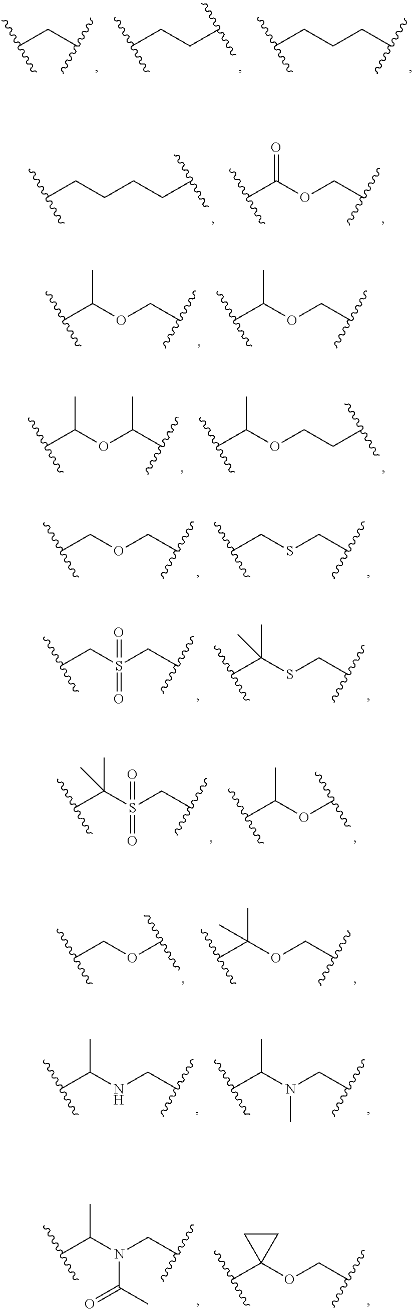

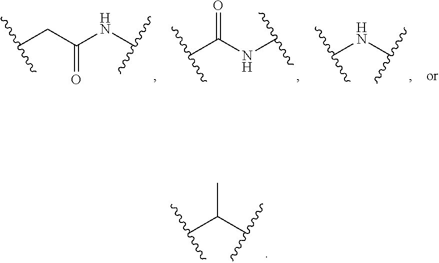

In some embodiments, L¹ is

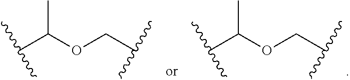

In some embodiments, L¹ is

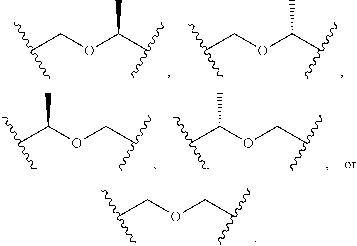

In some embodiments, L³ is

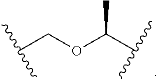 [SMILES: *CO[C@H](*)C]

In some embodiments, L¹ is

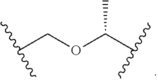 [SMILES: *CO[C@@H](*)C]

In some embodiments, L¹ is

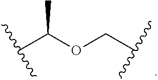 [SMILES: *CO[C@@H](*)C]

In some embodiments, L¹ is

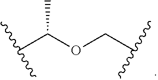 [SMILES: *CO[C@H](*)C]

In some embodiments, L¹ is

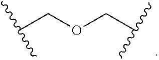 [SMILES: *COC*]

In some embodiments, L¹ is selected from those depicted in the compounds of Table 1, below.

As defined generally above, R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur).

In some embodiments, R¹ is hydrogen. In some embodiments, R¹ is an optionally substituted C₁₋₆ aliphatic group. In some embodiments, R¹ is methyl. In some embodiments, R¹ is ethyl. In some embodiments, R¹ is isopropyl.

In some embodiments, R¹ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R¹ is an optionally substituted cyclic group selected from a 3-8 membered saturated or partially unsaturated monocyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 3-8 membered saturated or partially unsaturated monocyclic heterocyclic ring (having 1-2 heteroatoms independently selected from nitrogen, oxygen, and sulfur). In some embodiments, R¹ is an optionally substituted 5-6 membered monocyclic heteroaromatic ring (having 1-4 heteroatoms independently selected from nitrogen, oxygen, and sulfur). In some embodiments, R¹ is an optionally substituted 8-10 membered bicyclic heteroaromatic ring (having 1-5 heteroatoms independently selected from nitrogen, oxygen, and sulfur). In some embodiments, R¹ is an optionally substituted cyclic group selected from phenyl, cyclohexyl, cyclopentyl, cyclobutyl, cyclopropyl, cycloheptyl, oxazolyl, pyridinyl, pyridazinyl, 1,3,4-oxadiazolyl, 1,2,3-triazolyl, pyrazolyl, and tetrahydropyranyl. In some embodiments, R¹ is optionally substituted phenyl. In some embodiments, R¹ is optionally substituted cyclohexyl.

In some embodiments, R¹ is an optionally substituted 7-12 membered saturated or partially unsaturated bicyclic heterocyclic ring that is optionally bridged bicyclic or spirocyclic (having 1-4 heteroatoms independently selected from nitrogen, oxygen, and sulfur). In some embodiments, R¹ is an optionally substituted a 7-12 membered saturated or partially unsaturated bridged bicyclic carbocyclic ring. In some embodiments, R¹ is an optionally substituted 7-12 membered bridge bicyclic carbocyclic ring or an optionally substituted 7-12 membered bridged bicyclic heterocyclic ring (having 1-4 heteroatoms independently selected from nitrogen, oxygen, and sulfur). In some embodiments, R¹ is optionally substituted oxabicyclo[2.2.2]octanyl. In some embodiments, R¹ is optionally substituted bicyclo[2.2.2]octanyl.

In some embodiments, R¹ is

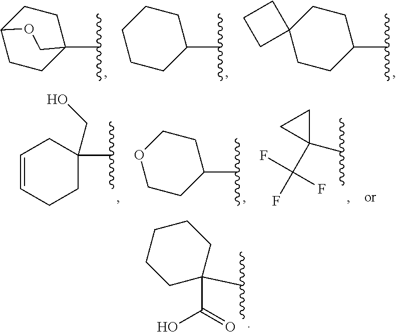

In some embodiments, R¹ is

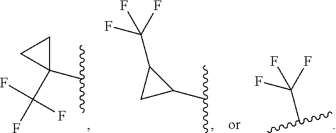 [SMILES: *C1(C(F)(F)F)CC1 ; *C1CC1C(F)(F)F ; *C(F)(F)F]

In some embodiments, R¹ is selected from those depicted in the compounds of Table 1, below.

As defined generally above, R² is hydrogen, an optionally substituted C₁₋₆ aliphatic group, —C₁₋₆ alkylene-OR, —C₁₋₃ alkylene-O—C₁₋₃ alkylene-R, —C(O)OR, —C(O)NR₂, —S(O)₂R, —S(O)₂NR₂, —S(O)R, —P(O)R₂, —C(O)NRS(O)₂R, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R³ is hydrogen; or R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur).

In some embodiments, R² is hydrogen, an optionally substituted C₁₋₆ aliphatic group, —C₁₋₆ alkylene-OR, —C₁₋₃ alkylene-O—C₁₋₃ alkylene-R, —C(O)OR, or —C(O)NR₂; and R³ is hydrogen; or R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur).

Alternatively, R² is hydrogen, an optionally substituted C₁₋₆ aliphatic group, —C₁₋₆alkylene-OR, —C₁₋₃ alkylene-O—C₁₋₃ alkylene-R, —C(O)OR, —C(O)NR₂, —S(O)₂R, —S(O)₂NR₂, —S(O)R, —P(O)R₂, —C(O)NRS(O)₂R, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R³ is hydrogen; or R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur).

In some embodiments, R² is hydrogen, an optionally substituted C₁₋₆ aliphatic group, —C₁₋₆ alkylene-OR, —C₁₋₃ alkylene-O—C₁₋₃ alkylene-R, —C(O)OR, —C(O)NR₂, —S(O)₂R, —S(O)₂NR₂, —S(O)R, —P(O)R₂, —C(O)NRS(O)₂R, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R³ is hydrogen.

In some embodiments, R² is an optionally substituted C₁₋₆ aliphatic group, —C₁₋₆ alkylene-OR, —C(O)OR, —C(O)NR₂, —C(O)NRS(O)₂R, or an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen.

In some embodiments, R² is an optionally substituted C₁₋₆ aliphatic group, —C₁₋₆ alkylene-OR, —C(O)OR, —C(O)NR₂, —C(O)NRS(O)₂R, or an optionally substituted phenyl; and R³ is hydrogen.

In some embodiments, R² is hydrogen, an optionally substituted C₁₋₆ aliphatic group, —C₁₋₆ alkylene-OR, —C₁₋₃ alkylene-O—C₁₋₃ alkylene-R, —C(O)OR, or —C(O)NR₂; and R³ is hydrogen. In some embodiments, R² is hydrogen, methyl, —CH₂OR, —CH₂OCH₂R, —C(O)OR, or —C(O)NR₂; and R³ is hydrogen. In some embodiments, R² is hydrogen. In some embodiments, R² is an optionally substituted C₁₋₆ aliphatic group. In some embodiments, R² is methyl. In some embodiments, R² is —C₁₋₆ alkylene-OR. In some embodiments, R² is —CH₂OR. In some embodiments, R² is —CH₂OCH₂R. In some embodiments, R² is —C(O)OR. In some embodiments, R² is —C(O)NR₂. In some embodiments, R² is —C(O)NR₂, wherein the two R groups, taken together with the intervening nitrogen atom, form an optionally substituted 4-7 membered saturated, partially unsaturated, or heteroaryl ring (having 0-3 heteroatoms, in addition to the nitrogen, independently selected from nitrogen, oxygen, and sulfur). In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur). In some embodiments, R² is —C(O)NR₂, wherein the two R groups, taken together with the intervening nitrogen atom, form an optionally substituted 4-7 membered saturated ring, selected from a piperidinyl, morpholinyl, piperazinyl, azetindinyl, pyrrolidinyl, azaspiro[3.3]heptanyl, and diazaspiro[3.3]heptanyl.

In some embodiments, R² is —S(O)₂R, —S(O)₂NR₂, —S(O)R, or —P(O)R₂. In some embodiments, R² is —S(O)₂R. In some embodiments, R² is —S(O)₂NR₂. In some embodiments, R² is —S(O)R. In some embodiments, R² is —P(O)R₂. In some embodiments, R² is —S(O₂)CH₃. In some embodiments, R² is —P(O)(CH₃)₂.

In some embodiments, R² is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R² is an optionally substituted cyclic group selected from a 3-8 membered saturated or partially unsaturated monocyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R² is an optionally substituted 3-8 membered saturated or partially unsaturated monocyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 3-8 membered saturated or partially unsaturated monocyclic heterocyclic ring (having 1-2 heteroatoms independently selected from nitrogen, oxygen, and sulfur). In some embodiments, R² is an optionally substituted 5-6 membered monocyclic heteroaromatic ring (having 1-4 heteroatoms independently selected from nitrogen, oxygen, and sulfur). In some embodiments, R² is an optionally substituted 8-10 membered bicyclic heteroaromatic ring (having 1-5 heteroatoms independently selected from nitrogen, oxygen, and sulfur). In some embodiments, R² is a tetrahydrofuranyl. In some embodiments, R² is a dioxanyl. In some embodiments, R⁸ is a furanyl. In some embodiments, R² is an oxadiazolyl. In some embodiments, R² is an oxazolyl.

In some embodiments, R² is selected from those depicted in the compounds of Table 1, below.

In some embodiments, R³ is hydrogen and R² is a substituent in Table A:

TABLE A
Exemplary R² substituents

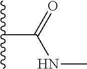 [SMILES: *C(=O)NC]

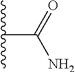 [SMILES: *C(N)=O]

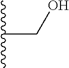 [SMILES: *CO]

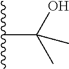 [SMILES: *C(C)(C)O]

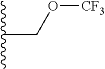 [SMILES: *COC(F)(F)F]

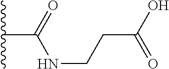 [SMILES: *C(=O)NCCC(=O)O]

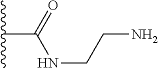 [SMILES: *C(=O)NCCN]

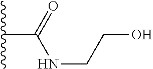 [SMILES: *C(=O)NCCO]

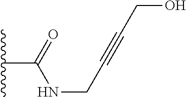 [SMILES: *C(=O)NCC#CCO]

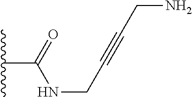 [SMILES: *C(=O)NCC#CCN]

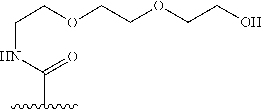 [SMILES: *C(=O)NCCOCCOCCO]

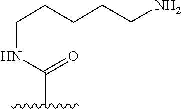 [SMILES: *C(=O)NCCCCCN]

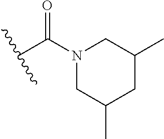 [SMILES: *C(=O)N1CC(C)CC(C)C1]

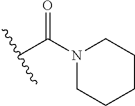 [SMILES: *C(=O)N1CCCCC1]

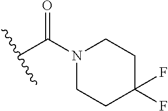 [SMILES: *C(=O)N1CCC(F)(F)CC1]

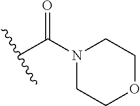 [SMILES: *C(=O)N1CCOCC1]

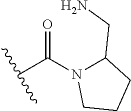 [SMILES: *C(=O)N1CCCC1CN]

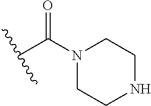 [SMILES: *C(=O)N1CCNCC1]

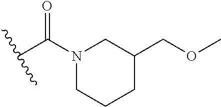 [SMILES: *C(=O)N1CCCC(COC)C1]

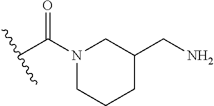 [SMILES: *C(=O)N1CCCC(CN)C1]

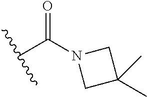 [SMILES: *C(=O)N1CC(C)(C)C1]

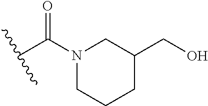 [SMILES: *C(=O)N1CCCC(CO)C1]

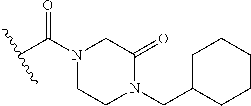 [SMILES: *C(=O)N1CCN(CC2CCCCC2)C(=O)C1]

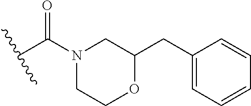 [SMILES: *C(=O)N1CCOC(Cc2ccccc2)C1]

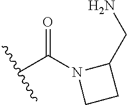 [SMILES: *C(=O)N1CCC1CN]

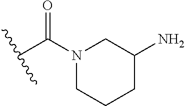 [SMILES: *C(=O)N1CCCC(N)C1]

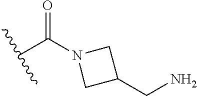 [SMILES: *C(=O)N1CC(CN)C1]

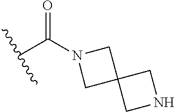 [SMILES: *C(=O)N1CC2(CNC2)C1]

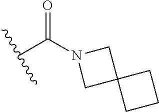 [SMILES: *C(=O)N1CC2(CCC2)C1]

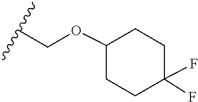 [SMILES: *COC1CCC(F)(F)CC1]

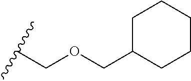 [SMILES: *COCC1CCCCC1]

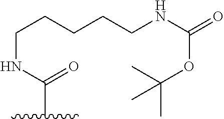 [SMILES: *C(=O)NCCCCCNC(=O)OC(C)(C)C]

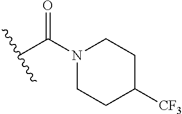 [SMILES: *C(=O)N1CCC(C(F)(F)F)CC1]

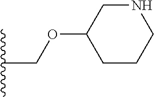 [SMILES: *COC1CCCNC1]

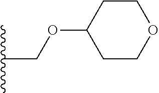 [SMILES: *COC1CCOCC1]

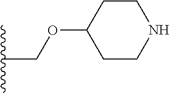 [SMILES: *COC1CCNCC1]

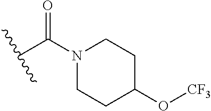 [SMILES: *C(=O)N1CCC(OC(F)(F)F)CC1]

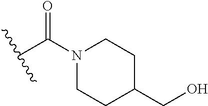 [SMILES: *C(=O)N1CCC(CO)CC1]

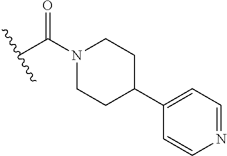 [SMILES: *C(=O)N1CCC(c2ccncc2)CC1]

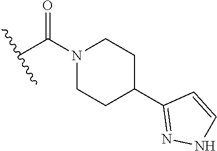 [SMILES: *C(=O)N1CCC(c2cc[nH]n2)CC1]

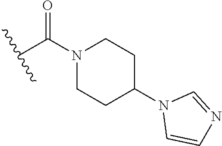 [SMILES: *C(=O)N1CCC(n2ccnc2)CC1]

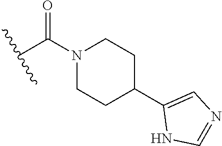 [SMILES: *C(=O)N1CCC(c2cnc[nH]2)CC1]

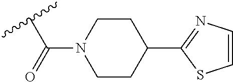 [SMILES: *C(=O)N1CCC(c2nccs2)CC1]

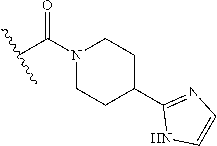 [SMILES: *C(=O)N1CCC(c2ncc[nH]2)CC1]

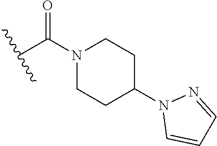 [SMILES: *C(=O)N1CCC(n2cccn2)CC1]

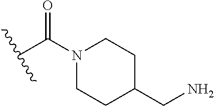 [SMILES: *C(=O)N1CCC(CN)CC1]

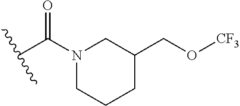 [SMILES: *C(=O)N1CCCC(COC(F)(F)F)C1]

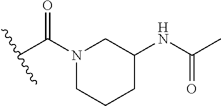 [SMILES: *C(=O)N1CCCC(NC(C)=O)C1]

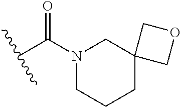 [SMILES: *C(=O)N1CCCC2(COC2)C1]

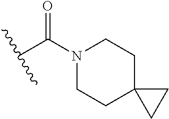 [SMILES: *C(=O)N1CCC2(CC1)CC2]

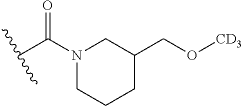 [SMILES: *C(=O)N1CCCC(COC([2H])([2H])[2H])C1]

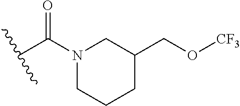 [SMILES: *C(=O)N1CCCC(COC(F)(F)F)C1]

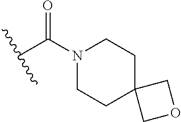 [SMILES: *C(=O)N1CCC2(CC1)COC2]

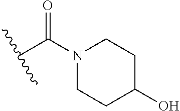 [SMILES: *C(=O)N1CCC(O)CC1]

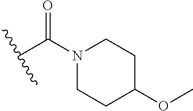 [SMILES: *C(=O)N1CCC(OC)CC1]

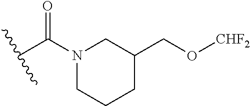 [SMILES: *C(=O)N1CCCC(COC(F)F)C1]

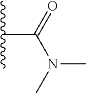 [SMILES: *C(=O)N(C)C]

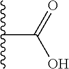 [SMILES: *C(=O)O]

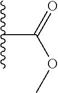 [SMILES: *C(=O)OC]

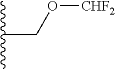 [SMILES: *COC(F)F]

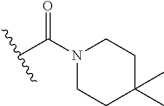 [SMILES: *C(=O)N1CCC(C)(C)CC1]

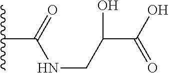 [SMILES: *C(=O)NCC(O)C(=O)O]

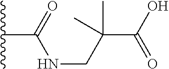 [SMILES: *C(=O)NCC(C)(C)C(=O)O]

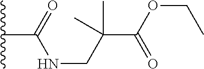 [SMILES: *C(=O)NCC(C)(C)C(=O)OCC]

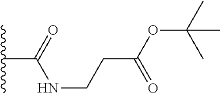 [SMILES: *C(=O)NCCC(=O)OC(C)(C)C]

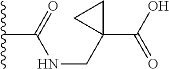 [SMILES: *C(=O)NCC1(C(=O)O)CC1]

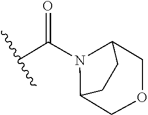 [SMILES: *C(=O)N1C2CCC1COC2]

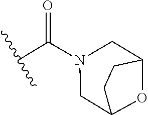 [SMILES: *C(=O)N1CC2CCC(C1)O2]

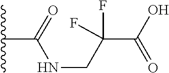 [SMILES: *C(=O)NCC(F)(F)C(=O)O]

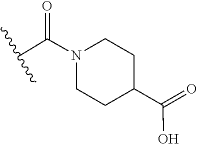 [SMILES: *C(=O)N1CCC(C(=O)O)CC1]

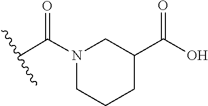 [SMILES: *C(=O)N1CCCC(C(=O)O)C1]

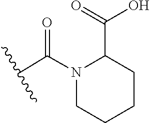 [SMILES: *C(=O)N1CCCCC1C(=O)O]

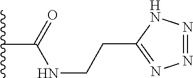 [SMILES: *C(=O)NCCc1nnn[nH]1]

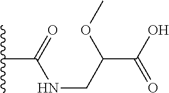 [SMILES: *C(=O)NCC(OC)C(=O)O]

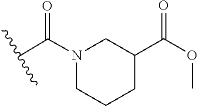 [SMILES: *C(=O)N1CCCC(C(=O)OC)C1]

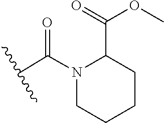 [SMILES: *C(=O)N1CCCCC1C(=O)OC]

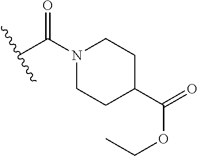 [SMILES: *C(=O)N1CCC(C(=O)OCC)CC1]

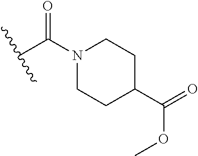 [SMILES: *C(=O)N1CCC(C(=O)OC)CC1]

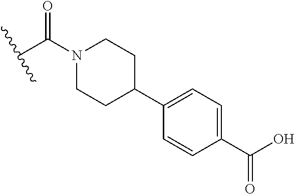 [SMILES: *C(=O)N1CCC(c2ccc(C(=O)O)cc2)CC1]

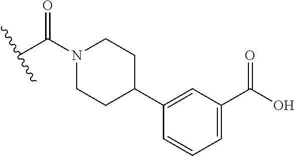 [SMILES: *C(=O)N1CCC(c2cccc(C(=O)O)c2)CC1]

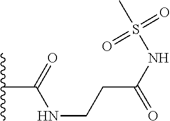 [SMILES: *C(=O)NCCC(=O)NS(C)(=O)=O]

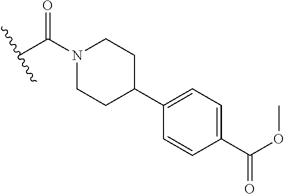 [SMILES: *C(=O)N1CCC(c2ccc(C(=O)OC)cc2)CC1]

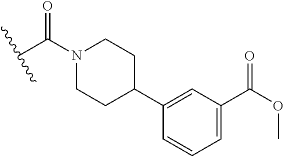 [SMILES: *C(=O)N1CCC(c2cccc(C(=O)OC)c2)CC1]

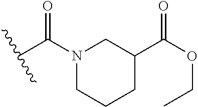 [SMILES: *C(=O)N1CCCC(C(=O)OCC)C1]

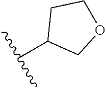 [SMILES: *C1CCOC1]

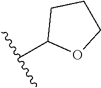 [SMILES: *C1CCCO1]

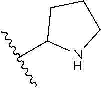 [SMILES: *C1CCCN1]

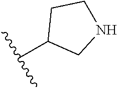 [SMILES: *C1CCNC1]

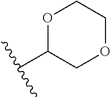 [SMILES: *C1COCCO1]

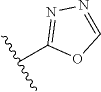 [SMILES: *c1nnco1]

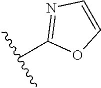 [SMILES: *c1ncco1]

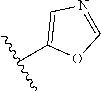 [SMILES: *c1cnco1]

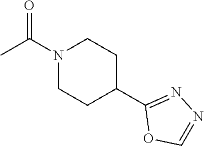

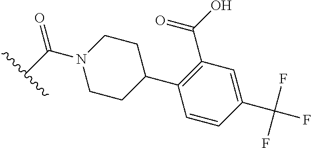 [SMILES: *C(=O)N1CCC(c2ccc(C(F)(F)F)cc2C(=O)O)CC1]

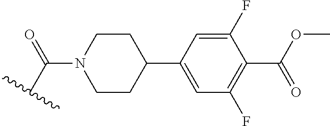 [SMILES: *C(=O)N1CCC(c2cc(F)c(C(=O)OC)c(F)c2)CC1]

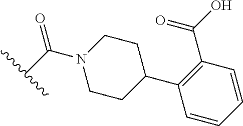 [SMILES: *C(=O)N1CCC(c2ccccc2C(=O)O)CC1]

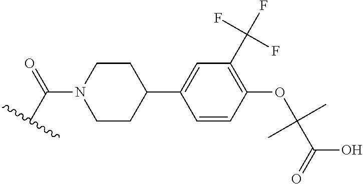 [SMILES: *C(=O)N1CCC(c2ccc(OC(C)(C)C(=O)O)c(C(F)(F)F)c2)CC1]

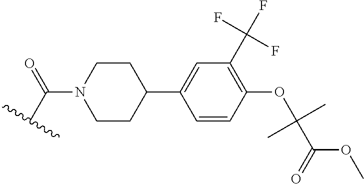 [SMILES: *C(=O)N1CCC(c2ccc(OC(C)(C)C(=O)OC)c(C(F)(F)F)c2)CC1]

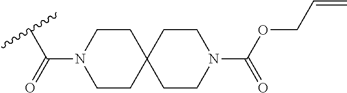 [SMILES: *C(=O)N1CCC2(CC1)CCN(C(=O)OCC=C)CC2]

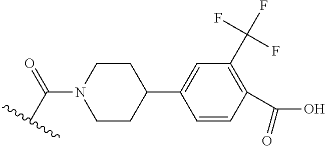 [SMILES: *C(=O)N1CCC(c2ccc(C(=O)O)c(C(F)(F)F)c2)CC1]

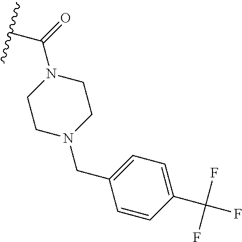 [SMILES: *C(=O)N1CCN(Cc2ccc(C(F)(F)F)cc2)CC1]

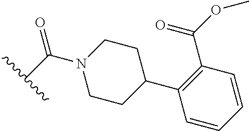 [SMILES: *C(=O)N1CCC(c2ccccc2C(=O)OC)CC1]

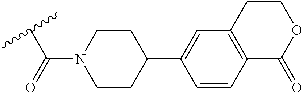 [SMILES: *C(=O)N1CCC(c2ccc3c(c2)CCOC3=O)CC1]

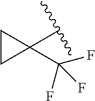 [SMILES: *C1(C(F)(F)F)CC1]

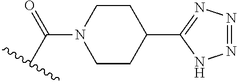 [SMILES: *C(=O)N1CCC(c2nnn[nH]2)CC1]

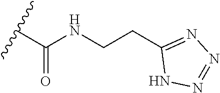 [SMILES: *C(=O)NCCc1nnn[nH]1]

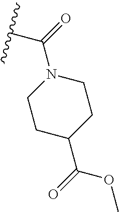 [SMILES: *C(=O)N1CCC(C(=O)OC)CC1]

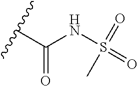 [SMILES: *C(=O)NS(C)(=O)=O]

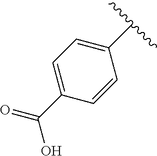 [SMILES: *c1ccc(C(=O)O)cc1]

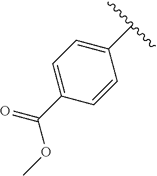 [SMILES: *c1ccc(C(=O)OC)cc1]

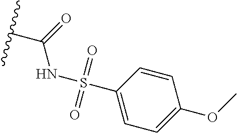 [SMILES: *C(=O)NS(=O)(=O)c1ccc(OC)cc1]

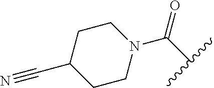 [SMILES: *C(=O)N1CCC(C#N)CC1]

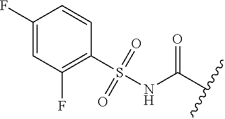 [SMILES: *C(=O)NS(=O)(=O)c1ccc(F)cc1F]

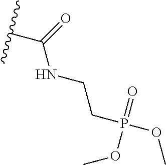 [SMILES: *C(=O)NCCP(=O)(OC)OC]

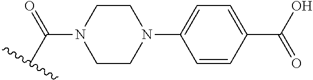 [SMILES: *C(=O)N1CCN(c2ccc(C(=O)O)cc2)CC1]

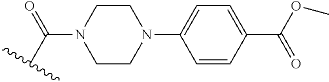 [SMILES: *C(=O)N1CCN(c2ccc(C(=O)OC)cc2)CC1]

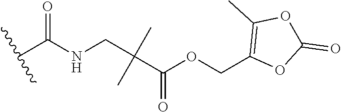 [SMILES: *C(=O)NCC(C)(C)C(=O)OCc1oc(=O)oc1C]

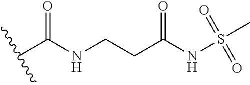 [SMILES: *C(=O)NCCC(=O)NS(C)(=O)=O]

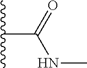 [SMILES: *C(=O)NC]

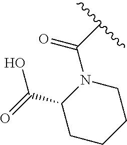 [SMILES: *C(=O)N1CCCC[C@@H]1C(=O)O]

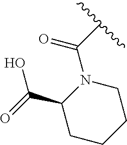 [SMILES: *C(=O)N1CCCC[C@H]1C(=O)O]

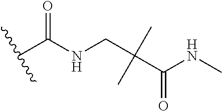 [SMILES: *C(=O)NCC(C)(C)C(=O)NC]

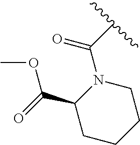 [SMILES: *C(=O)N1CCCC[C@H]1C(=O)OC]

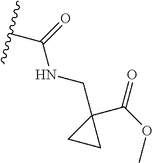 [SMILES: *C(=O)NCC1(C(=O)OC)CC1]

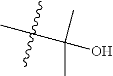 [SMILES: *C(C)(C)O]

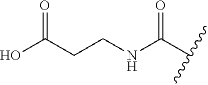 [SMILES: *C(=O)NCCC(=O)O]

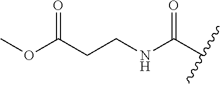 [SMILES: *C(=O)NCCC(=O)OC]

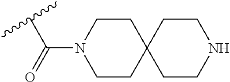 [SMILES: *C(=O)N1CCC2(CCNCC2)CC1]

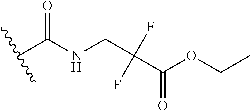 [SMILES: *C(=O)NCC(F)(F)C(=O)OCC]

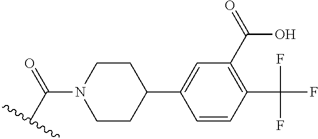 [SMILES: *C(=O)N1CCC(c2ccc(C(F)(F)F)c(C(=O)O)c2)CC1]

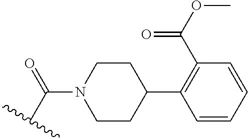 [SMILES: *C(=O)N1CCC(c2ccccc2C(=O)OC)CC1]

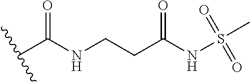 [SMILES: *C(=O)NCCC(=O)NS(C)(=O)=O]

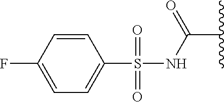 [SMILES: *C(=O)NS(=O)(=O)c1ccc(F)cc1]

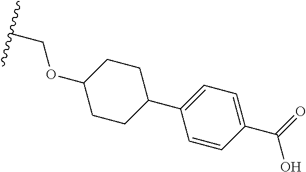 [SMILES: *COC1CCC(c2ccc(C(=O)O)cc2)CC1]

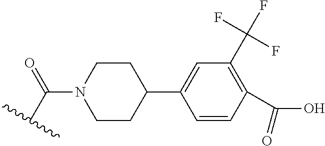 [SMILES: *C(=O)N1CCC(c2ccc(C(=O)O)c(C(F)(F)F)c2)CC1]

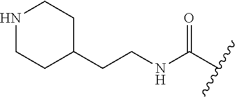 [SMILES: *C(=O)NCCC1CCNCC1]

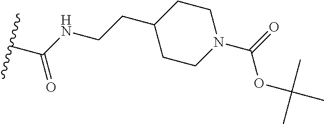 [SMILES: *C(=O)NCCC1CCN(C(=O)OC(C)(C)C)CC1]

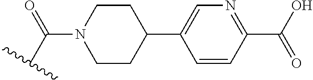 [SMILES: *C(=O)N1CCC(c2ccc(C(=O)O)nc2)CC1]

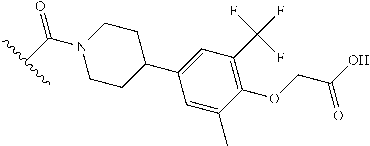 [SMILES: *C(=O)N1CCC(c2cc(C)c(OCC(=O)O)c(C(F)(F)F)c2)CC1]

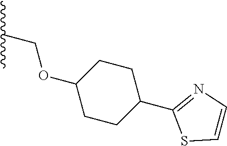 [SMILES: *COC1CCC(c2nccs2)CC1]

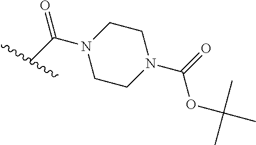 [SMILES: *C(=O)N1CCN(C(=O)OC(C)(C)C)CC1]

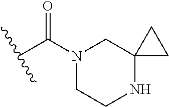 [SMILES: *C(=O)N1CCNC2(CC2)C1]

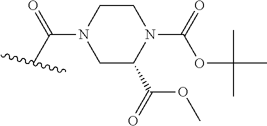 [SMILES: *C(=O)N1CCN(C(=O)OC(C)(C)C)[C@H](C(=O)OC)C1]

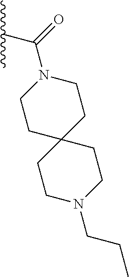 [SMILES: *C(=O)N1CCC2(CCN(CCC)CC2)CC1]

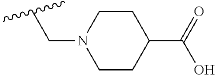 [SMILES: *CN1CCC(C(=O)O)CC1]

In some embodiments, R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur). In some embodiments, R² and R³ together with the intervening carbon atom form an optionally substituted 3-7 membered saturated or partially unsaturated carbocyclic ring. In some embodiments, R² and R³ together with the intervening carbon atom form an optionally substituted 3-7 membered saturated carbocyclic ring. In some embodiments, R² and R³ together with the intervening carbon atom form an optionally substituted 3-7 membered saturated or partially unsaturated heterocyclic ring (having 1-2 heteroatoms independently selected from nitrogen, oxygen, and sulfur). In some embodiments, R² and R³ together with the intervening carbon atom form an optionally substituted 3-7 membered saturated heterocyclic ring (having 1-2 heteroatoms independently selected from nitrogen, oxygen, and sulfur). In some embodiments, R² and R³ together with the intervening carbon atom form an optionally substituted oxetanyl, cyclopropyl, cyclobutyl, cyclopentyl, tetrahydropyranyl, piperidinyl, piperazinyl, morpholinyl, pyrrolidinyl, or 1,4-oxazepanyl. In some embodiments, R² and R³ form a cyclic group selected from those depicted in the compounds of Table 1, below.

As defined generally above, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), and R⁵ is hydrogen; or R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, independently selected from nitrogen, oxygen, and sulfur).

In some embodiments, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), and R⁵ is hydrogen. In some embodiments, R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), and a 5-6 membered monocyclic heteroaromatic ring (having 1-4 heteroatoms independently selected from nitrogen, oxygen, and sulfur). In some embodiments, R⁴ is an optionally substituted 3-8 membered saturated or partially unsaturated monocyclic carbocyclic ring. In some embodiments, R⁴ is an optionally substituted 7-12 membered saturated or partially unsaturated bicyclic carbocyclic ring. In some embodiments, R⁴ is an optionally substituted phenyl. In some embodiments, R⁴ is an optionally substituted 8-10 membered bicyclic aromatic carbocyclic ring. In some embodiments, R⁴ is an optionally substituted 3-8 membered saturated or partially unsaturated monocyclic heterocyclic ring (having 1-2 heteroatoms independently selected from nitrogen, oxygen, and sulfur). In some embodiments, R⁴ is an optionally substituted 5-6 membered monocyclic heteroaromatic ring (having 1-4 heteroatoms independently selected from nitrogen, oxygen, and sulfur). In some embodiments, R⁴ is an optionally substituted cyclic group selected from phenyl, piperidinyl, tetrahydropyranyl, 1,4-oxazepanyl, oxazolyl, cyclobutyl, cyclopentyl, or pyrrolidinyl. In some embodiments, R⁴ is selected from those depicted in the compounds of Table 1, below.

In some embodiments, R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur). In some embodiments, R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur). In some embodiments, R⁴ and R⁵ together with the intervening nitrogen atom form an optionally substituted heteroaryl ring (having 0-3 heteroatoms, in addition to the nitrogen, independently selected from nitrogen, oxygen, and sulfur).

In some embodiments, R⁴ and R⁵ together with the intervening nitrogen atom form an optionally substituted 6 membered saturated heterocyclic ring (having 0 or 1 additional nitrogen atoms, in addition to the intervening nitrogen). In some embodiments, R⁴ and R⁵ together with the intervening nitrogen atom form an optionally substituted 6 membered saturated heterocyclic ring.

In some embodiments, R⁴ and R⁵ together with the intervening nitrogen atom form an optionally substituted 6 membered saturated heterocyclic ring (having 1 additional nitrogen atom, in addition to the intervening nitrogen).

In some embodiments, R⁴ and R⁵ together with the intervening nitrogen atom form an optionally substituted cyclic group selected from piperindinyl, piperazinyl, morpholinyl, and pyrrolidinyl. In some embodiments, R⁴ and R⁵ together with the intervening nitrogen atom form a substituted cyclic group, wherein the cyclic group is substituted with one or more groups selected from —C₁₋₆ alkylene-phenyl, —O—C₁₋₆ alkylene-phenyl, —C₁₋₆ alkylene-cyclohexyl, —O—C₁₋₆ alkylene-cyclohexyl, —C₁₋₆ alkylene-COOH, —C₁₋₆ alkylene-C(O)O—(C₁₋₄alkyl), —C₁₋₆ alkylene-C(O)NHS(O)₂—(C₁₋₄alkyl). In some embodiments, R⁴ and R⁵ form a cyclic group selected from those depicted in the compounds of Table 1, below.

In some embodiments, R^{A} is a substituent of Table B:

TABLE B
Exemplary R^{A} substituents

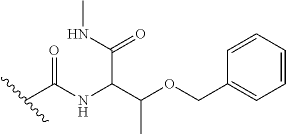 [SMILES: *C(=O)NC(C(=O)NC)C(C)OCc1ccccc1]

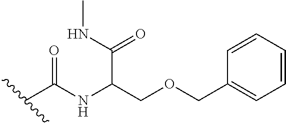 [SMILES: *C(=O)NC(COCc1ccccc1)C(=O)NC]

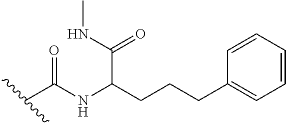 [SMILES: *C(=O)NC(CCCc1ccccc1)C(=O)NC]

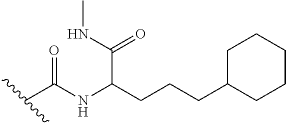 [SMILES: *C(=O)NC(CCCC1CCCCC1)C(=O)NC]

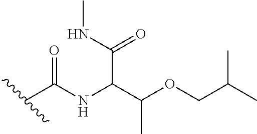 [SMILES: *C(=O)NC(C(=O)NC)C(C)OCC(C)C]

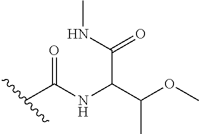 [SMILES: *C(=O)NC(C(=O)NC)C(C)OC]

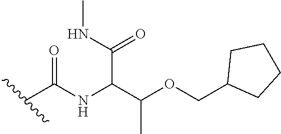 [SMILES: *C(=O)NC(C(=O)NC)C(C)OCC1CCCC1]

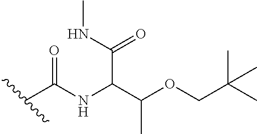 [SMILES: *C(=O)NC(C(=O)NC)C(C)OCC(C)(C)C]

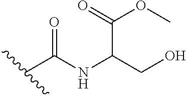 [SMILES: *C(=O)NC(CO)C(=O)OC]

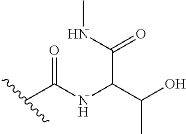 [SMILES: *C(=O)NC(C(=O)NC)C(C)O]

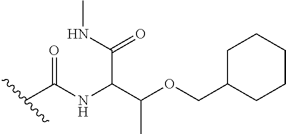 [SMILES: *C(=O)NC(C(=O)NC)C(C)OCC1CCCCC1]

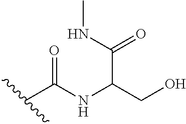 [SMILES: *C(=O)NC(CO)C(=O)NC]

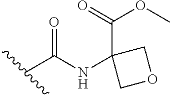 [SMILES: *C(=O)NC1(C(=O)OC)COC1]

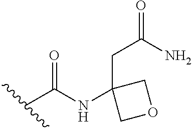 [SMILES: *C(=O)NC1(CC(N)=O)COC1]

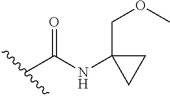 [SMILES: *C(=O)NC1(COC)CC1]

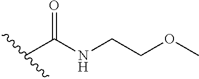 [SMILES: *C(=O)NCCOC]

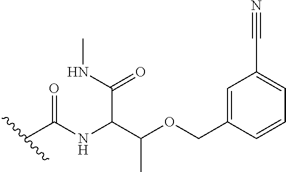 [SMILES: *C(=O)NC(C(=O)NC)C(C)OCc1cccc(C#N)c1]

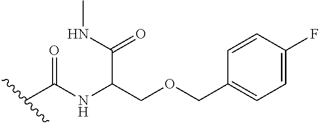 [SMILES: *C(=O)NC(COCc1ccc(F)cc1)C(=O)NC]

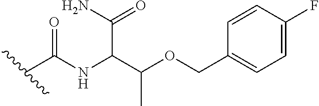 [SMILES: *C(=O)NC(C(N)=O)C(C)OCc1ccc(F)cc1]

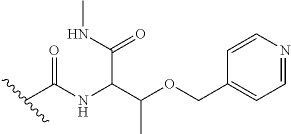 [SMILES: *C(=O)NC(C(=O)NC)C(C)OCc1ccncc1]

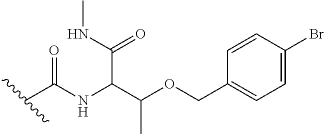 [SMILES: *C(=O)NC(C(=O)NC)C(C)OCc1ccc(Br)cc1]

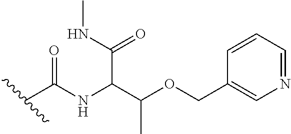 [SMILES: *C(=O)NC(C(=O)NC)C(C)OCc1cccnc1]

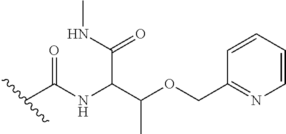 [SMILES: *C(=O)NC(C(=O)NC)C(C)OCc1ccccn1]

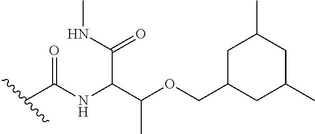 [SMILES: *C(=O)NC(C(=O)NC)C(C)OCC1CC(C)CC(C)C1]

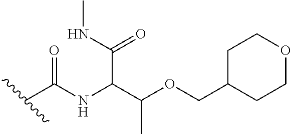 [SMILES: *C(=O)NC(C(=O)NC)C(C)OCC1CCOCC1]

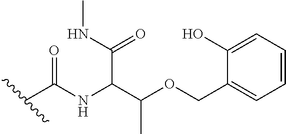 [SMILES: *C(=O)NC(C(=O)NC)C(C)OCc1ccccc1O]

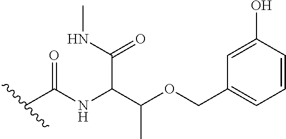 [SMILES: *C(=O)NC(C(=O)NC)C(C)OCc1cccc(O)c1]

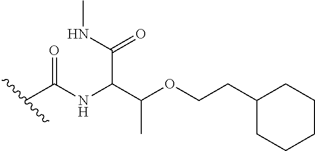 [SMILES: *C(=O)NC(C(=O)NC)C(C)OCCC1CCCCC1]

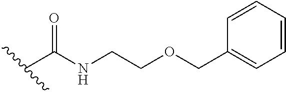 [SMILES: *C(=O)NCCOCc1ccccc1]

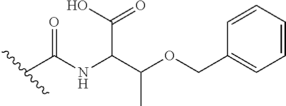 [SMILES: *C(=O)NC(C(=O)O)C(C)OCc1ccccc1]

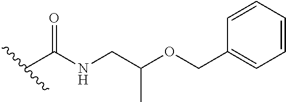 [SMILES: *C(=O)NCC(C)OCc1ccccc1]

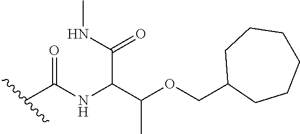 [SMILES: *C(=O)NC(C(=O)NC)C(C)OCC1CCCCCC1]

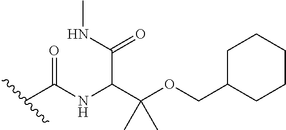 [SMILES: *C(=O)NC(C(=O)NC)C(C)(C)OCC1CCCCC1]

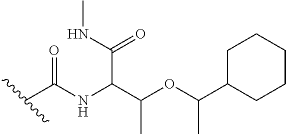 [SMILES: *C(=O)NC(C(=O)NC)C(C)OC(C)C1CCCCC1]

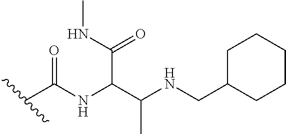 [SMILES: *C(=O)NC(C(=O)NC)C(C)NCC1CCCCC1]

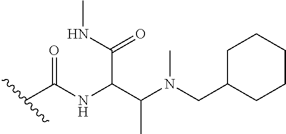 [SMILES: *C(=O)NC(C(=O)NC)C(C)N(C)CC1CCCCC1]

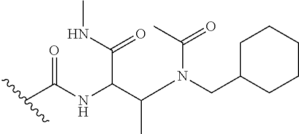 [SMILES: *C(=O)NC(C(=O)NC)C(C)N(CC1CCCCC1)C(C)=O]

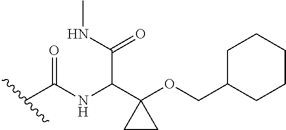 [SMILES: *C(=O)NC(C(=O)NC)C1(OCC2CCCCC2)CC1]

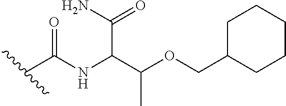 [SMILES: *C(=O)NC(C(N)=O)C(C)OCC1CCCCC1]

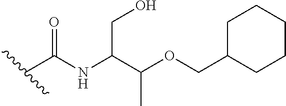 [SMILES: *C(=O)NC(CO)C(C)OCC1CCCCC1]

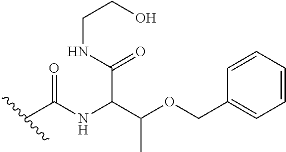 [SMILES: *C(=O)NC(C(=O)NCCO)C(C)OCc1ccccc1]

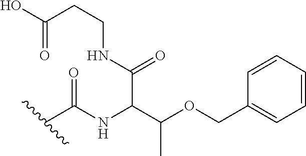 [SMILES: *C(=O)NC(C(=O)NCCC(=O)O)C(C)OCc1ccccc1]

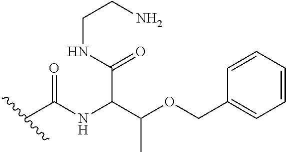 [SMILES: *C(=O)NC(C(=O)NCCN)C(C)OCc1ccccc1]

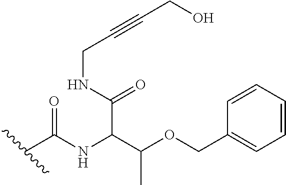 [SMILES: *C(=O)NC(C(=O)NCC#CCO)C(C)OCc1ccccc1]

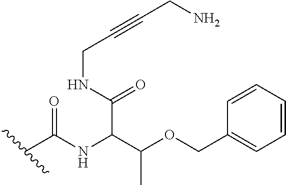 [SMILES: *C(=O)NC(C(=O)NCC#CCN)C(C)OCc1ccccc1]

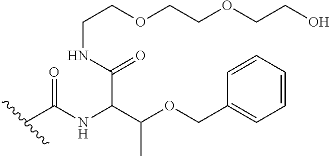 [SMILES: *C(=O)NC(C(=O)NCCOCCOCCO)C(C)OCc1ccccc1]

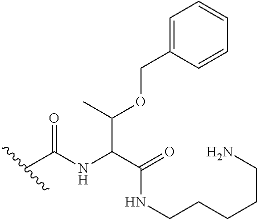 [SMILES: *C(=O)NC(C(=O)NCCCCCN)C(C)OCc1ccccc1]

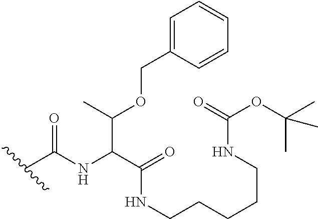 [SMILES: *C(=O)NC(C(=O)NCCCCCNC(=O)OC(C)(C)C)C(C)OCc1ccccc1]

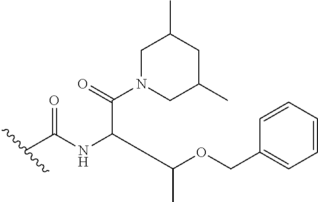 [SMILES: *C(=O)NC(C(=O)N1CC(C)CC(C)C1)C(C)OCc1ccccc1]

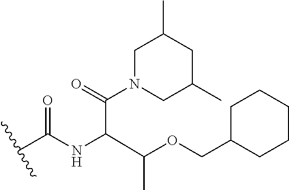 [SMILES: *C(=O)NC(C(=O)N1CC(C)CC(C)C1)C(C)OCC1CCCCC1]

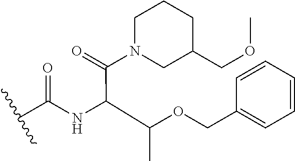 [SMILES: *C(=O)NC(C(=O)N1CCCC(COC)C1)C(C)OCc1ccccc1]

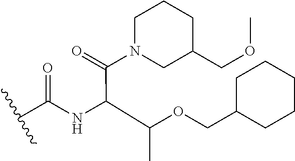 [SMILES: *C(=O)NC(C(=O)N1CCCC(COC)C1)C(C)OCC1CCCCC1]

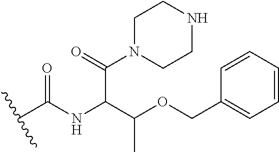 [SMILES: *C(=O)NC(C(=O)N1CCNCC1)C(C)OCc1ccccc1]

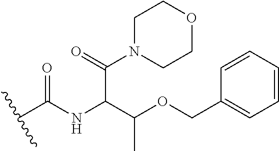 [SMILES: *C(=O)NC(C(=O)N1CCOCC1)C(C)OCc1ccccc1]

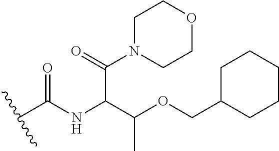 [SMILES: *C(=O)NC(C(=O)N1CCOCC1)C(C)OCC1CCCCC1]

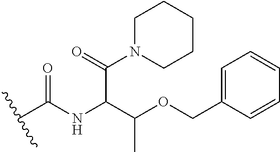 [SMILES: *C(=O)NC(C(=O)N1CCCCC1)C(C)OCc1ccccc1]

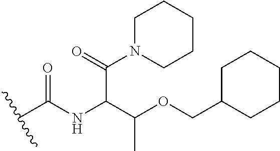 [SMILES: *C(=O)NC(C(=O)N1CCCCC1)C(C)OCC1CCCCC1]

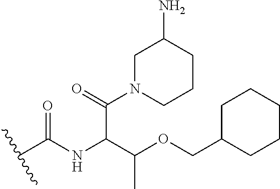 [SMILES: *C(=O)NC(C(=O)N1CCCC(N)C1)C(C)OCC1CCCCC1]

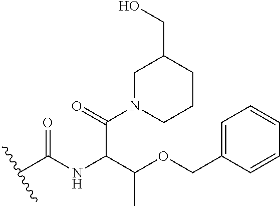 [SMILES: *C(=O)NC(C(=O)N1CCCC(CO)C1)C(C)OCc1ccccc1]

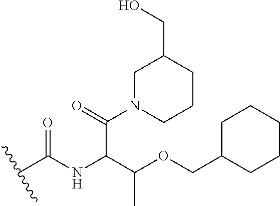 [SMILES: *C(=O)NC(C(=O)N1CCCC(CO)C1)C(C)OCC1CCCCC1]

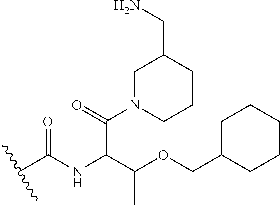 [SMILES: *C(=O)NC(C(=O)N1CCCC(CN)C1)C(C)OCC1CCCCC1]

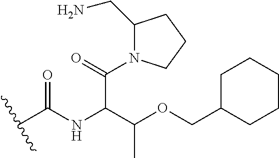 [SMILES: *C(=O)NC(C(=O)N1CCCC1CN)C(C)OCC1CCCCC1]

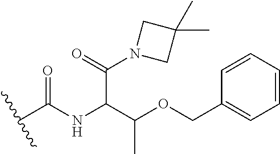 [SMILES: *C(=O)NC(C(=O)N1CC(C)(C)C1)C(C)OCc1ccccc1]

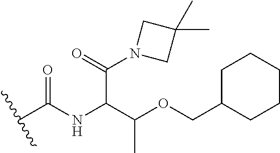 [SMILES: *C(=O)NC(C(=O)N1CC(C)(C)C1)C(C)OCC1CCCCC1]

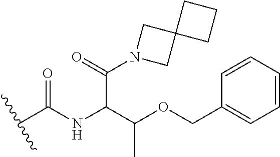 [SMILES: *C(=O)NC(C(=O)N1CC2(CCC2)C1)C(C)OCc1ccccc1]

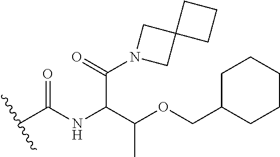 [SMILES: *C(=O)NC(C(=O)N1CC2(CCC2)C1)C(C)OCC1CCCCC1]

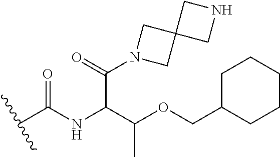 [SMILES: *C(=O)NC(C(=O)N1CC2(CNC2)C1)C(C)OCC1CCCCC1]

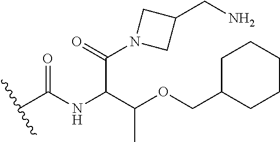 [SMILES: *C(=O)NC(C(=O)N1CC(CN)C1)C(C)OCC1CCCCC1]

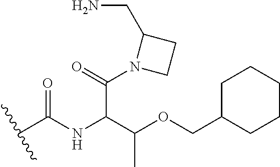 [SMILES: *C(=O)NC(C(=O)N1CCC1CN)C(C)OCC1CCCCC1]

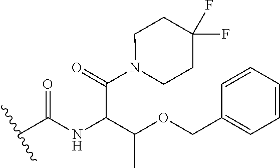 [SMILES: *C(=O)NC(C(=O)N1CCC(F)(F)CC1)C(C)OCc1ccccc1]

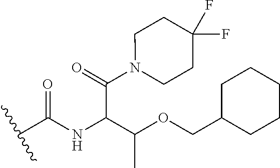 [SMILES: *C(=O)NC(C(=O)N1CCC(F)(F)CC1)C(C)OCC1CCCCC1]

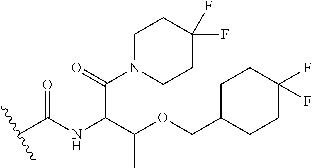 [SMILES: *C(=O)NC(C(=O)N1CCC(F)(F)CC1)C(C)OCC1CCC(F)(F)CC1]

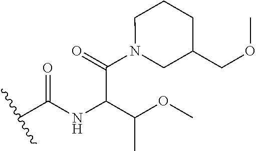 [SMILES: *C(=O)NC(C(=O)N1CCCC(COC)C1)C(C)OC]

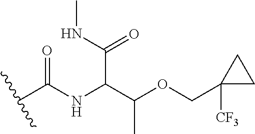 [SMILES: *C(=O)NC(C(=O)NC)C(C)OCC1(C(F)(F)F)CC1]

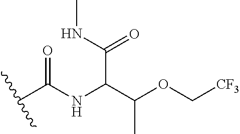 [SMILES: *C(=O)NC(C(=O)NC)C(C)OCC(F)(F)F]

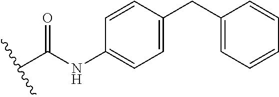 [SMILES: *C(=O)Nc1ccc(Cc2ccccc2)cc1]

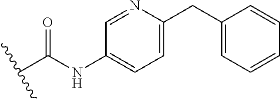 [SMILES: *C(=O)Nc1ccc(Cc2ccccc2)nc1]

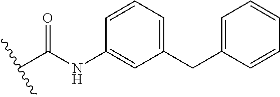 [SMILES: *C(=O)Nc1cccc(Cc2ccccc2)c1]

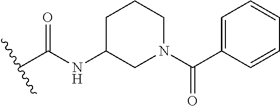 [SMILES: *C(=O)NC1CCCN(C(=O)c2ccccc2)C1]

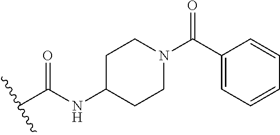 [SMILES: *C(=O)NC1CCN(C(=O)c2ccccc2)CC1]

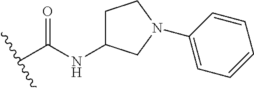 [SMILES: *C(=O)NC1CCN(c2ccccc2)C1]

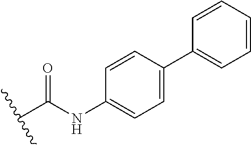 [SMILES: *C(=O)Nc1ccc(-c2ccccc2)cc1]

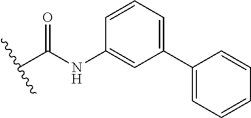 [SMILES: *C(=O)Nc1cccc(-c2ccccc2)c1]

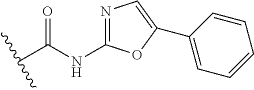 [SMILES: *C(=O)Nc1ncc(-c2ccccc2)o1]

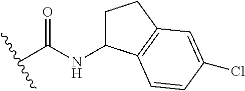 [SMILES: *C(=O)NC1CCc2cc(Cl)ccc21]

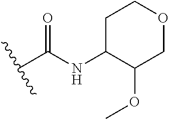 [SMILES: *C(=O)NC1CCOCC1OC]

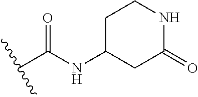 [SMILES: *C(=O)NC1CCNC(=O)C1]

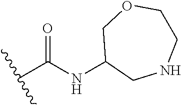 [SMILES: *C(=O)NC1CNCCOC1]

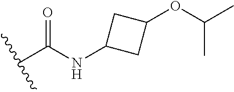 [SMILES: *C(=O)NC1CC(OC(C)C)C1]

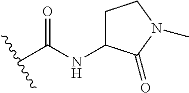 [SMILES: *C(=O)NC1CCN(C)C1=O]

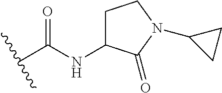 [SMILES: *C(=O)NC1CCN(C2CC2)C1=O]

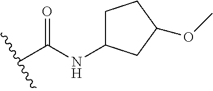 [SMILES: *C(=O)NC1CCC(OC)C1]

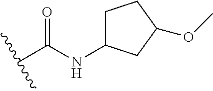 [SMILES: *C(=O)NC1CCC(OC)C1]

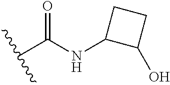 [SMILES: *C(=O)NC1CCC1O]

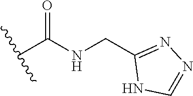 [SMILES: *C(=O)NCc1nnc[nH]1]

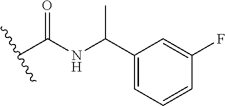 [SMILES: *C(=O)NC(C)c1cccc(F)c1]

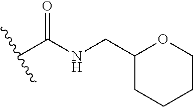 [SMILES: *C(=O)NCC1CCCCO1]

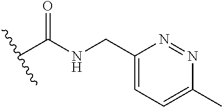 [SMILES: *C(=O)NCc1ccc(C)nn1]

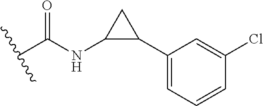 [SMILES: *C(=O)NC1CC1c1cccc(Cl)c1]

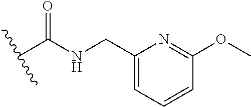 [SMILES: *C(=O)NCc1cccc(OC)n1]

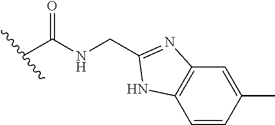 [SMILES: *C(=O)NCc1nc2cc(C)ccc2[nH]1]

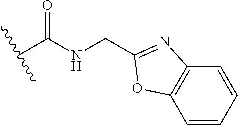 [SMILES: *C(=O)NCc1nc2ccccc2o1]

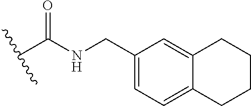 [SMILES: *C(=O)NCc1ccc2c(c1)CCCC2]

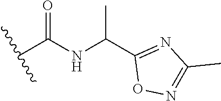 [SMILES: *C(=O)NC(C)c1nc(C)no1]

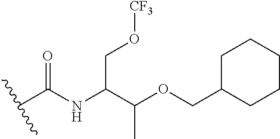 [SMILES: *C(=O)NC(COC(F)(F)F)C(C)OCC1CCCCC1]

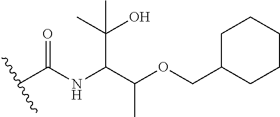 [SMILES: *C(=O)NC(C(C)OCC1CCCCC1)C(C)(C)O]

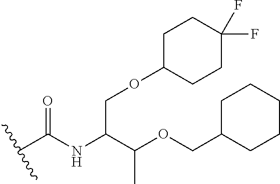 [SMILES: *C(=O)NC(COC1CCC(F)(F)CC1)C(C)OCC1CCCCC1]

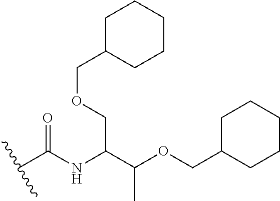 [SMILES: *C(=O)NC(COCC1CCCCC1)C(C)OCC1CCCCC1]

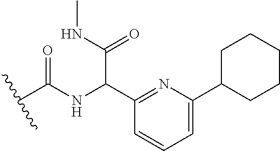 [SMILES: *C(=O)NC(C(=O)NC)c1cccc(C2CCCCC2)n1]

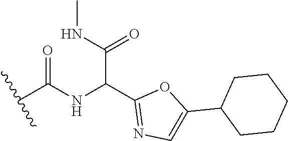 [SMILES: *C(=O)NC(C(=O)NC)c1ncc(C2CCCCC2)o1]

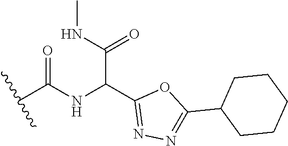 [SMILES: *C(=O)NC(C(=O)NC)c1nnc(C2CCCCC2)o1]

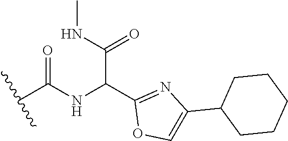 [SMILES: *C(=O)NC(C(=O)NC)c1nc(C2CCCCC2)co1]

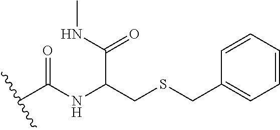 [SMILES: *C(=O)NC(CSCc1ccccc1)C(=O)NC]

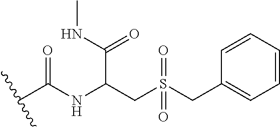 [SMILES: *C(=O)NC(CS(=O)(=O)Cc1ccccc1)C(=O)NC]

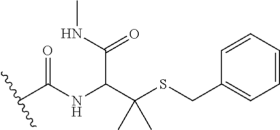 [SMILES: *C(=O)NC(C(=O)NC)C(C)(C)SCc1ccccc1]

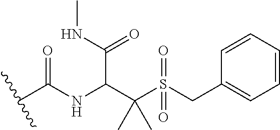 [SMILES: *C(=O)NC(C(=O)NC)C(C)(C)S(=O)(=O)Cc1ccccc1]

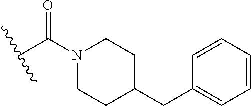 [SMILES: *C(=O)N1CCC(Cc2ccccc2)CC1]

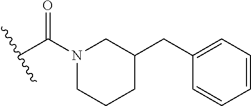 [SMILES: *C(=O)N1CCCC(Cc2ccccc2)C1]

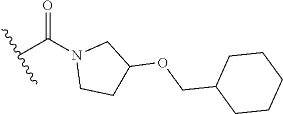 [SMILES: *C(=O)N1CCC(OCC2CCCCC2)C1]

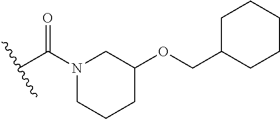 [SMILES: *C(=O)N1CCCC(OCC2CCCCC2)C1]

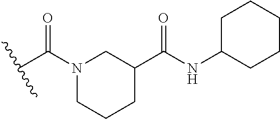 [SMILES: *C(=O)N1CCCC(C(=O)NC2CCCCC2)C1]

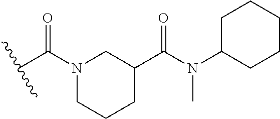 [SMILES: *C(=O)N1CCCC(C(=O)N(C)C2CCCCC2)C1]

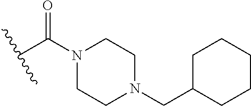 [SMILES: *C(=O)N1CCN(CC2CCCCC2)CC1]

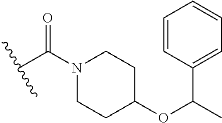 [SMILES: *C(=O)N1CCC(OC(C)c2ccccc2)CC1]

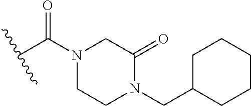 [SMILES: *C(=O)N1CCN(CC2CCCCC2)C(=O)C1]

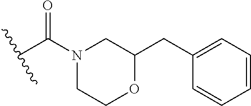 [SMILES: *C(=O)N1CCOC(Cc2ccccc2)C1]

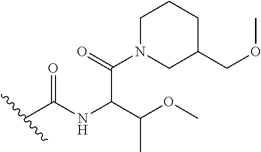 [SMILES: *C(=O)NC(C(=O)N1CCCC(COC)C1)C(C)OC]

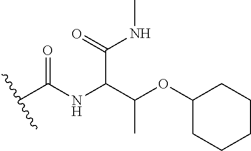 [SMILES: *C(=O)NC(C(=O)NC)C(C)OC1CCCCC1]

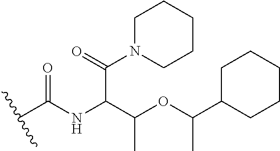 [SMILES: *C(=O)NC(C(=O)N1CCCCC1)C(C)OC(C)C1CCCCC1]

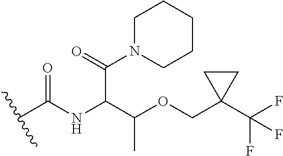 [SMILES: *C(=O)NC(C(=O)N1CCCCC1)C(C)OCC1(C(F)(F)F)CC1]

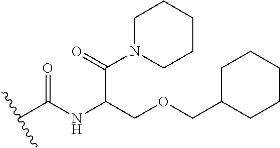 [SMILES: *C(=O)NC(COCC1CCCCC1)C(=O)N1CCCCC1]

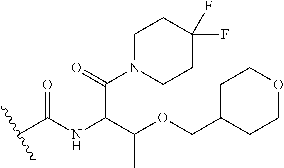 [SMILES: *C(=O)NC(C(=O)N1CCC(F)(F)CC1)C(C)OCC1CCOCC1]

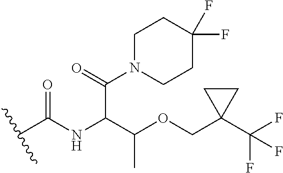 [SMILES: *C(=O)NC(C(=O)N1CCC(F)(F)CC1)C(C)OCC1(C(F)(F)F)CC1]

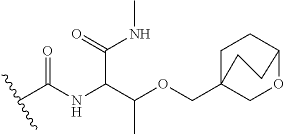 [SMILES: *C(=O)NC(C(=O)NC)C(C)OCC12CCC(CC1)OC2]

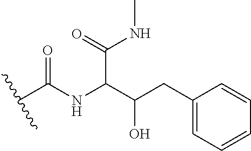 [SMILES: *C(=O)NC(C(=O)NC)C(O)Cc1ccccc1]

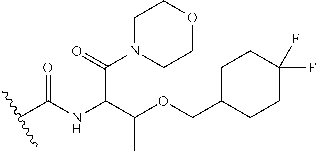 [SMILES: *C(=O)NC(C(=O)N1CCOCC1)C(C)OCC1CCC(F)(F)CC1]

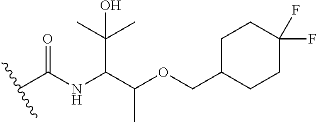 [SMILES: *C(=O)NC(C(C)OCC1CCC(F)(F)CC1)C(C)(C)O]

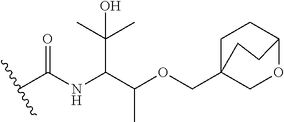 [SMILES: *C(=O)NC(C(C)OCC12CCC(CC1)OC2)C(C)(C)O]

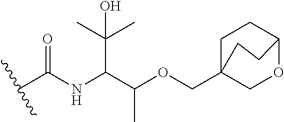 [SMILES: *C(=O)NC(C(C)OCC12CCC(CC1)OC2)C(C)(C)O]

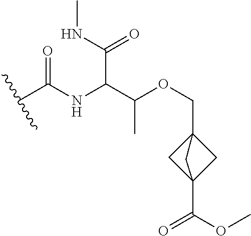 [SMILES: *C(=O)NC(C(=O)NC)C(C)OCC12CC(C(=O)OC)(C1)C2]

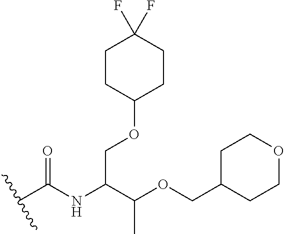 [SMILES: *C(=O)NC(COC1CCC(F)(F)CC1)C(C)OCC1CCOCC1]

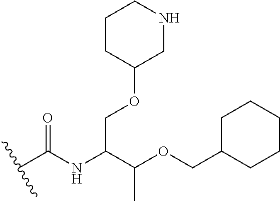 [SMILES: *C(=O)NC(COC1CCCNC1)C(C)OCC1CCCCC1]

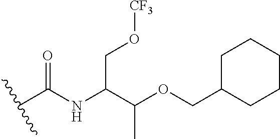 [SMILES: *C(=O)NC(COC(F)(F)F)C(C)OCC1CCCCC1]

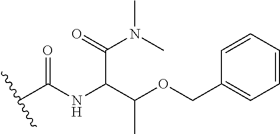 [SMILES: *C(=O)NC(C(=O)N(C)C)C(C)OCc1ccccc1]

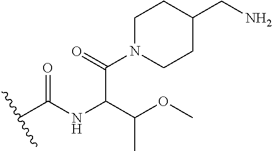 [SMILES: *C(=O)NC(C(=O)N1CCC(CN)CC1)C(C)OC]

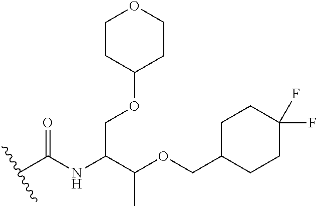 [SMILES: *C(=O)NC(COC1CCOCC1)C(C)OCC1CCC(F)(F)CC1]

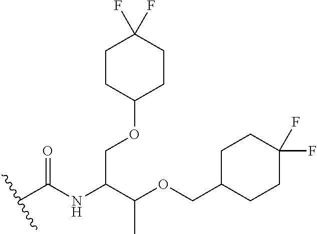 [SMILES: *C(=O)NC(COC1CCC(F)(F)CC1)C(C)OCC1CCC(F)(F)CC1]

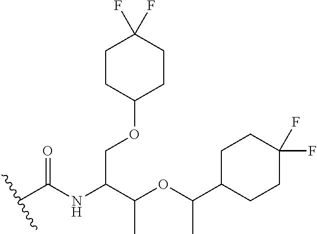 [SMILES: *C(=O)NC(COC1CCC(F)(F)CC1)C(C)OC(C)C1CCC(F)(F)CC1]

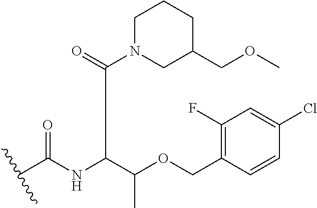 [SMILES: *C(=O)NC(C(=O)N1CCCC(COC)C1)C(C)OCc1ccc(Cl)cc1F]

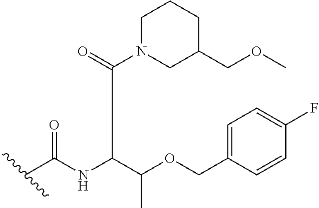 [SMILES: *C(=O)NC(C(=O)N1CCCC(COC)C1)C(C)OCc1ccc(F)cc1]

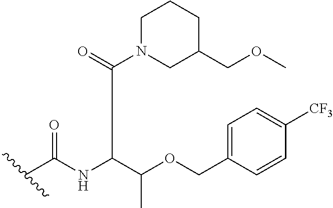 [SMILES: *C(=O)NC(C(=O)N1CCCC(COC)C1)C(C)OCc1ccc(C(F)(F)F)cc1]

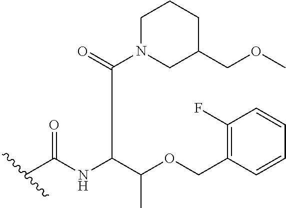 [SMILES: *C(=O)NC(C(=O)N1CCCC(COC)C1)C(C)OCc1ccccc1F]

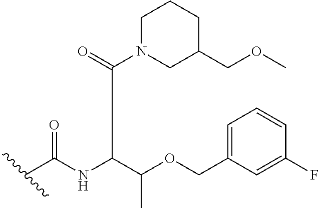 [SMILES: *C(=O)NC(C(=O)N1CCCC(COC)C1)C(C)OCc1cccc(F)c1]

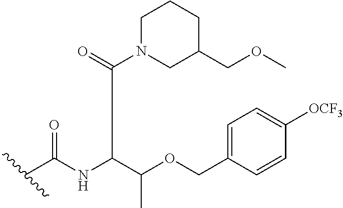 [SMILES: *C(=O)NC(C(=O)N1CCCC(COC)C1)C(C)OCc1ccc(OC(F)(F)F)cc1]

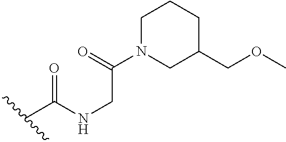 [SMILES: *C(=O)NCC(=O)N1CCCC(COC)C1]

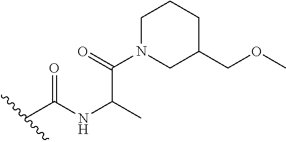 [SMILES: *C(=O)NC(C)C(=O)N1CCCC(COC)C1]

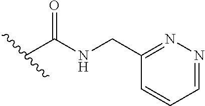 [SMILES: *C(=O)NCc1cccnn1]

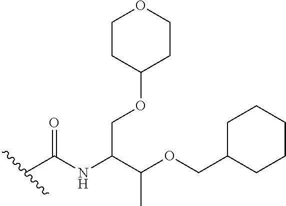 [SMILES: *C(=O)NC(COC1CCOCC1)C(C)OCC1CCCCC1]

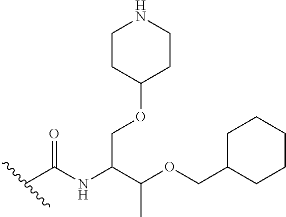 [SMILES: *C(=O)NC(COC1CCNCC1)C(C)OCC1CCCCC1]

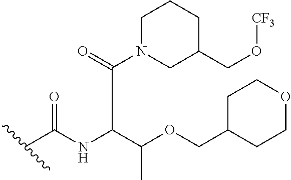 [SMILES: *C(=O)NC(C(=O)N1CCCC(COC(F)(F)F)C1)C(C)OCC1CCOCC1]

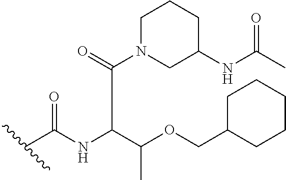 [SMILES: *C(=O)NC(C(=O)N1CCCC(NC(C)=O)C1)C(C)OCC1CCCCC1]

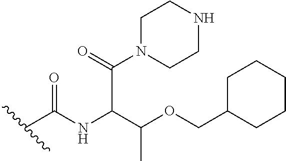 [SMILES: *C(=O)NC(C(=O)N1CCNCC1)C(C)OCC1CCCCC1]

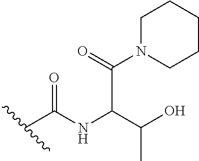 [SMILES: *C(=O)NC(C(=O)N1CCCCC1)C(C)O]

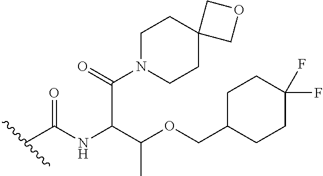 [SMILES: *C(=O)NC(C(=O)N1CCC2(CC1)COC2)C(C)OCC1CCC(F)(F)CC1]

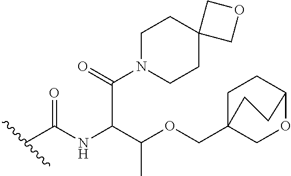 [SMILES: *C(=O)NC(C(=O)N1CCC2(CC1)COC2)C(C)OCC12CCC(CC1)OC2]

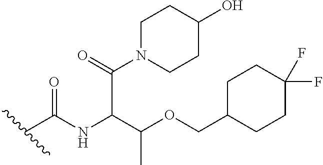 [SMILES: *C(=O)NC(C(=O)N1CCC(O)CC1)C(C)OCC1CCC(F)(F)CC1]

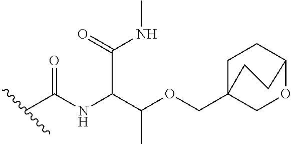 [SMILES: *C(=O)NC(C(=O)NC)C(C)OCC12CCC(CC1)OC2]

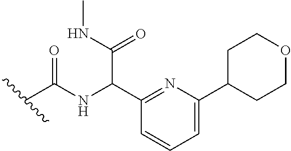 [SMILES: *C(=O)NC(C(=O)NC)c1cccc(C2CCOCC2)n1]

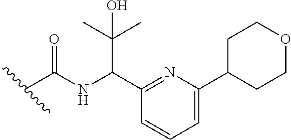 [SMILES: *C(=O)NC(c1cccc(C2CCOCC2)n1)C(C)(C)O]

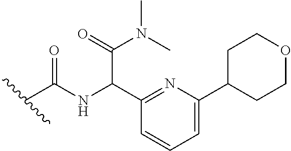 [SMILES: *C(=O)NC(C(=O)N(C)C)c1cccc(C2CCOCC2)n1]

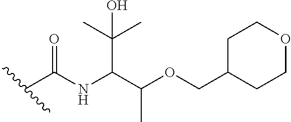 [SMILES: *C(=O)NC(C(C)OCC1CCOCC1)C(C)(C)O]

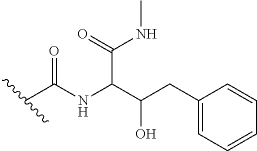 [SMILES: *C(=O)NC(C(=O)NC)C(O)Cc1ccccc1]

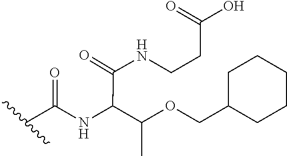 [SMILES: *C(=O)NC(C(=O)NCCC(=O)O)C(C)OCC1CCCCC1]

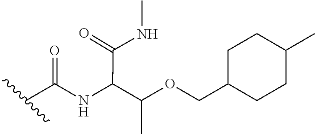 [SMILES: *C(=O)NC(C(=O)NC)C(C)OCC1CCC(C)CC1]

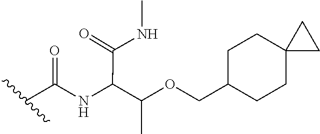 [SMILES: *C(=O)NC(C(=O)NC)C(C)OCC1CCC2(CC1)CC2]

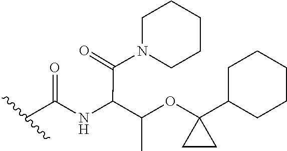 [SMILES: *C(=O)NC(C(=O)N1CCCCC1)C(C)OC1(C2CCCCC2)CC1]

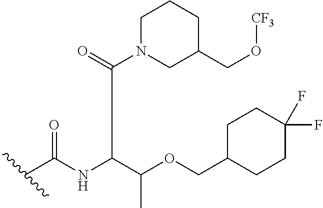 [SMILES: *C(=O)NC(C(=O)N1CCCC(COC(F)(F)F)C1)C(C)OCC1CCC(F)(F)CC1]

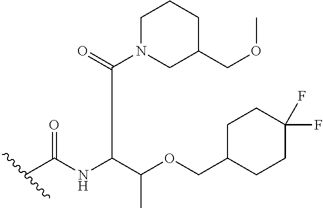 [SMILES: *C(=O)NC(C(=O)N1CCCC(COC)C1)C(C)OCC1CCC(F)(F)CC1]

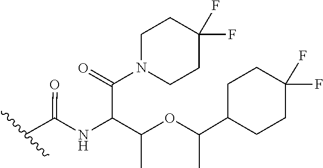 [SMILES: *C(=O)NC(C(=O)N1CCC(F)(F)CC1)C(C)OC(C)C1CCC(F)(F)CC1]

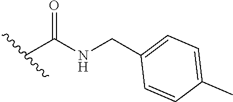 [SMILES: *C(=O)NCc1ccc(C)cc1]

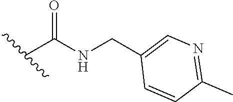 [SMILES: *C(=O)NCc1ccc(C)nc1]

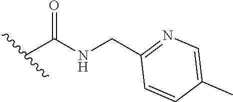 [SMILES: *C(=O)NCc1ccc(C)cn1]

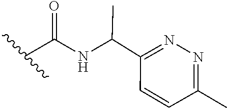 [SMILES: *C(=O)NC(C)c1ccc(C)nn1]

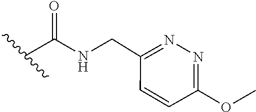 [SMILES: *C(=O)NCc1ccc(OC)nn1]

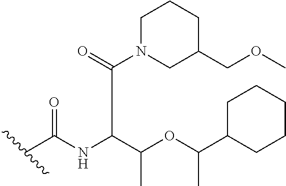 [SMILES: *C(=O)NC(C(=O)N1CCCC(COC)C1)C(C)OC(C)C1CCCCC1]

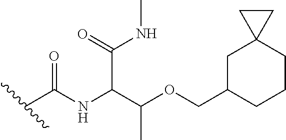 [SMILES: *C(=O)NC(C(=O)NC)C(C)OCC1CCCC2(CC2)C1]

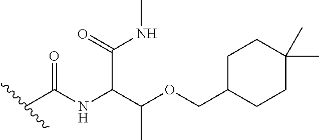 [SMILES: *C(=O)NC(C(=O)NC)C(C)OCC1CCC(C)(C)CC1]

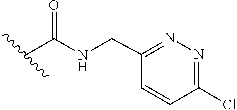 [SMILES: *C(=O)NCc1ccc(Cl)nn1]

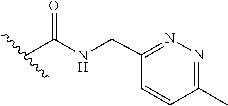 [SMILES: *C(=O)NCc1ccc(C)nn1]

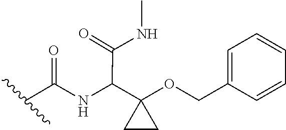 [SMILES: *C(=O)NC(C(=O)NC)C1(OCc2ccccc2)CC1]

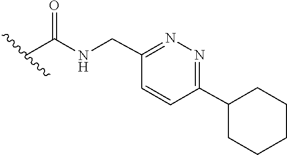 [SMILES: *C(=O)NCc1ccc(C2CCCCC2)nn1]

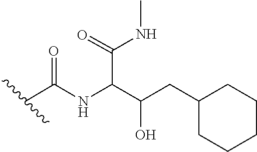 [SMILES: *C(=O)NC(C(=O)NC)C(O)CC1CCCCC1]

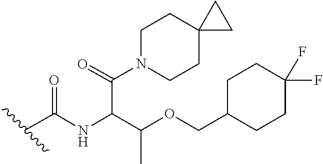 [SMILES: *C(=O)NC(C(=O)N1CCC2(CC1)CC2)C(C)OCC1CCC(F)(F)CC1]

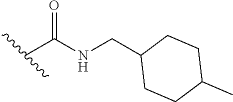 [SMILES: *C(=O)NCC1CCC(C)CC1]

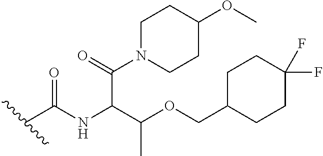 [SMILES: *C(=O)NC(C(=O)N1CCC(OC)CC1)C(C)OCC1CCC(F)(F)CC1]

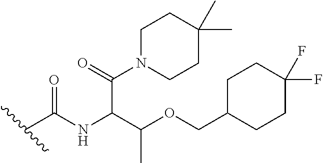 [SMILES: *C(=O)NC(C(=O)N1CCC(C)(C)CC1)C(C)OCC1CCC(F)(F)CC1]

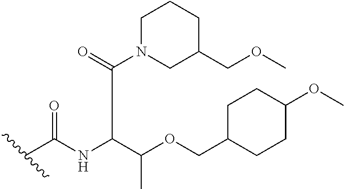 [SMILES: *C(=O)NC(C(=O)N1CCCC(COC)C1)C(C)OCC1CCC(OC)CC1]

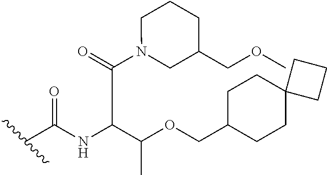 [SMILES: *C(=O)NC(C(=O)N1CCCC(COC)C1)C(C)OCC1CCC2(CCC2)CC1]

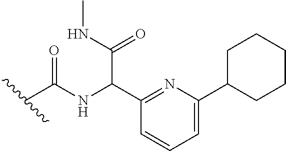 [SMILES: *C(=O)NC(C(=O)NC)c1cccc(C2CCCCC2)n1]

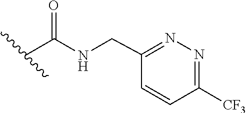 [SMILES: *C(=O)NCc1ccc(C(F)(F)F)nn1]

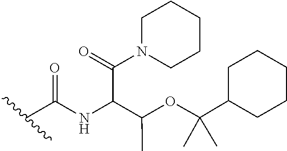 [SMILES: *C(=O)NC(C(=O)N1CCCCC1)C(C)OC(C)(C)C1CCCCC1]

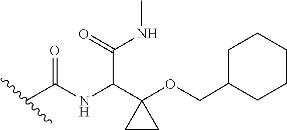 [SMILES: *C(=O)NC(C(=O)NC)C1(OCC2CCCCC2)CC1]

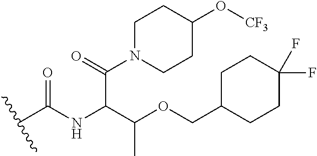 [SMILES: *C(=O)NC(C(=O)N1CCC(OC(F)(F)F)CC1)C(C)OCC1CCC(F)(F)CC1]

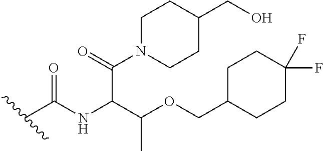 [SMILES: *C(=O)NC(C(=O)N1CCC(CO)CC1)C(C)OCC1CCC(F)(F)CC1]

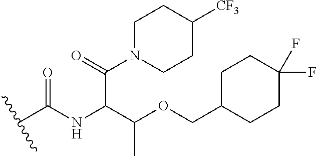 [SMILES: *C(=O)NC(C(=O)N1CCC(C(F)(F)F)CC1)C(C)OCC1CCC(F)(F)CC1]

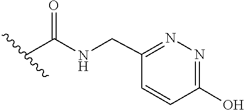 [SMILES: *C(=O)NCc1ccc(O)nn1]

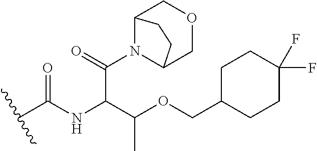 [SMILES: *C(=O)NC(C(=O)N1C2CCC1COC2)C(C)OCC1CCC(F)(F)CC1]

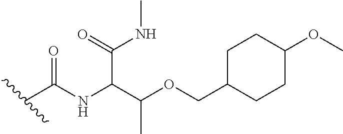 [SMILES: *C(=O)NC(C(=O)NC)C(C)OCC1CCC(OC)CC1]

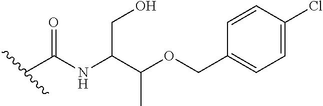 [SMILES: *C(=O)NC(CO)C(C)OCc1ccc(Cl)cc1]

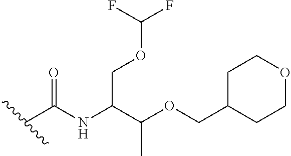 [SMILES: *C(=O)NC(COC(F)F)C(C)OCC1CCOCC1]

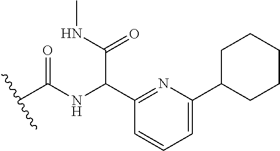 [SMILES: *C(=O)NC(C(=O)NC)c1cccc(C2CCCCC2)n1]

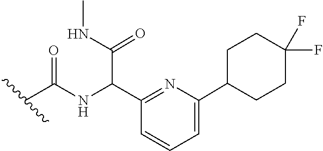 [SMILES: *C(=O)NC(C(=O)NC)c1cccc(C2CCC(F)(F)CC2)n1]

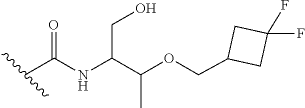 [SMILES: *C(=O)NC(CO)C(C)OCC1CC(F)(F)C1]

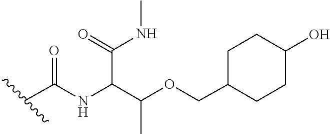 [SMILES: *C(=O)NC(C(=O)NC)C(C)OCC1CCC(O)CC1]

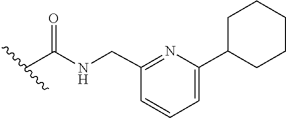 [SMILES: *C(=O)NCc1cccc(C2CCCCC2)n1]

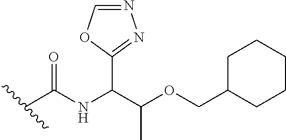 [SMILES: *C(=O)NC(c1nnco1)C(C)OCC1CCCCC1]

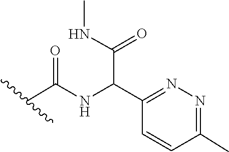 [SMILES: *C(=O)NC(C(=O)NC)c1ccc(C)nn1]

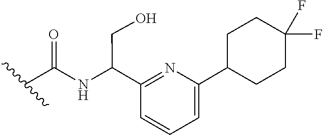 [SMILES: *C(=O)NC(CO)c1cccc(C2CCC(F)(F)CC2)n1]

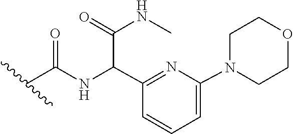 [SMILES: *C(=O)NC(C(=O)NC)c1cccc(N2CCOCC2)n1]

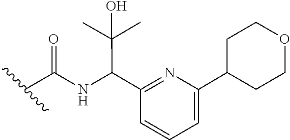 [SMILES: *C(=O)NC(c1cccc(C2CCOCC2)n1)C(C)(C)O]

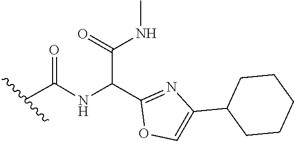 [SMILES: *C(=O)NC(C(=O)NC)c1nc(C2CCCCC2)co1]

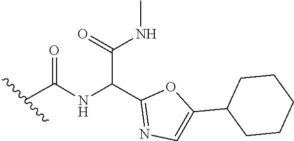 [SMILES: *C(=O)NC(C(=O)NC)c1ncc(C2CCCCC2)o1]

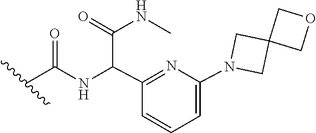 [SMILES: *C(=O)NC(C(=O)NC)c1cccc(N2CC3(COC3)C2)n1]

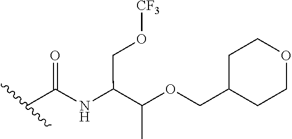 [SMILES: *C(=O)NC(COC(F)(F)F)C(C)OCC1CCOCC1]

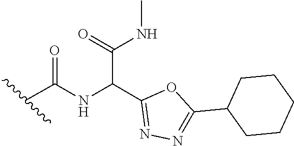 [SMILES: *C(=O)NC(C(=O)NC)c1nnc(C2CCCCC2)o1]

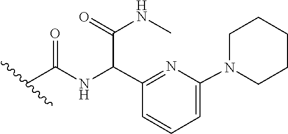 [SMILES: *C(=O)NC(C(=O)NC)c1cccc(N2CCCCC2)n1]

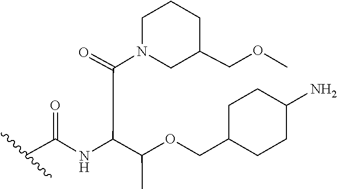 [SMILES: *C(=O)NC(C(=O)N1CCCC(COC)C1)C(C)OCC1CCC(N)CC1]

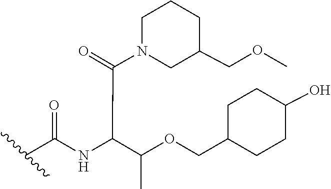 [SMILES: *C(=O)NC(C(=O)N1CCCC(COC)C1)C(C)OCC1CCC(O)CC1]

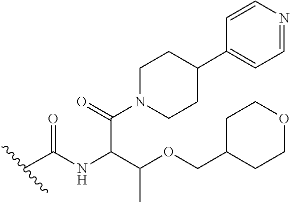 [SMILES: *C(=O)NC(C(=O)N1CCC(c2ccncc2)CC1)C(C)OCC1CCOCC1]

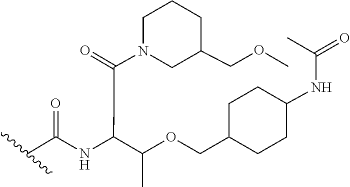 [SMILES: *C(=O)NC(C(=O)N1CCCC(COC)C1)C(C)OCC1CCC(NC(C)=O)CC1]

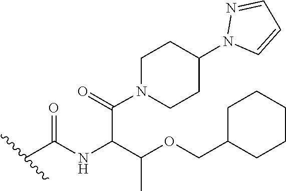 [SMILES: *C(=O)NC(C(=O)N1CCC(n2cccn2)CC1)C(C)OCC1CCCCC1]

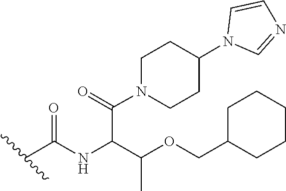 [SMILES: *C(=O)NC(C(=O)N1CCC(n2ccnc2)CC1)C(C)OCC1CCCCC1]

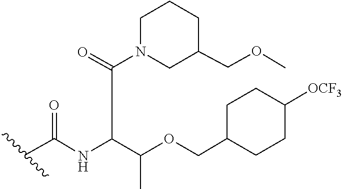 [SMILES: *C(=O)NC(C(=O)N1CCCC(COC)C1)C(C)OCC1CCC(OC(F)(F)F)CC1]

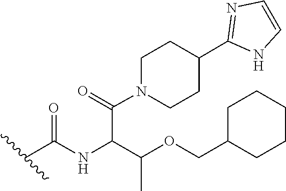 [SMILES: *C(=O)NC(C(=O)N1CCC(c2ncc[nH]2)CC1)C(C)OCC1CCCCC1]

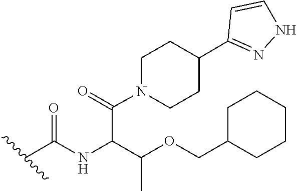 [SMILES: *C(=O)NC(C(=O)N1CCC(c2cc[nH]n2)CC1)C(C)OCC1CCCCC1]

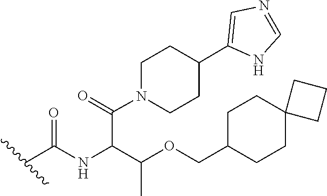 [SMILES: *C(=O)NC(C(=O)N1CCC(c2cnc[nH]2)CC1)C(C)OCC1CCC2(CCC2)CC1]

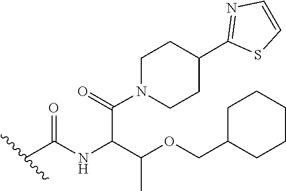 [SMILES: *C(=O)NC(C(=O)N1CCC(c2nccs2)CC1)C(C)OCC1CCCCC1]

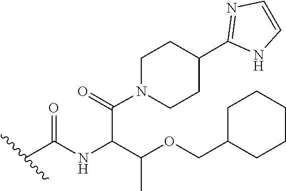 [SMILES: *C(=O)NC(C(=O)N1CCC(c2ncc[nH]2)CC1)C(C)OCC1CCCCC1]

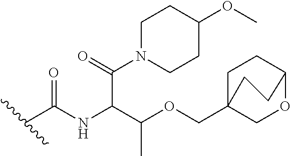 [SMILES: *C(=O)NC(C(=O)N1CCC(OC)CC1)C(C)OCC12CCC(CC1)OC2]

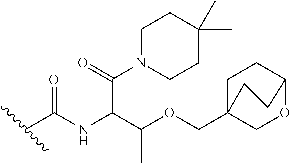 [SMILES: *C(=O)NC(C(=O)N1CCC(C)(C)CC1)C(C)OCC12CCC(CC1)OC2]

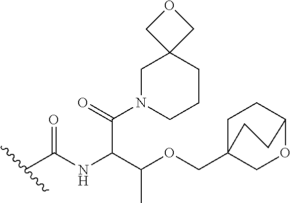 [SMILES: *C(=O)NC(C(=O)N1CCCC2(COC2)C1)C(C)OCC12CCC(CC1)OC2]

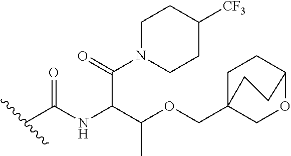 [SMILES: *C(=O)NC(C(=O)N1CCC(C(F)(F)F)CC1)C(C)OCC12CCC(CC1)OC2]

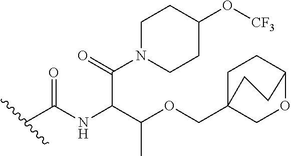 [SMILES: *C(=O)NC(C(=O)N1CCC(OC(F)(F)F)CC1)C(C)OCC12CCC(CC1)OC2]

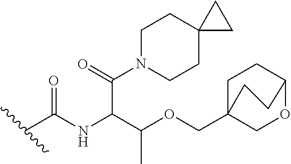 [SMILES: *C(=O)NC(C(=O)N1CCC2(CC1)CC2)C(C)OCC12CCC(CC1)OC2]

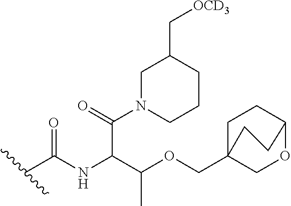

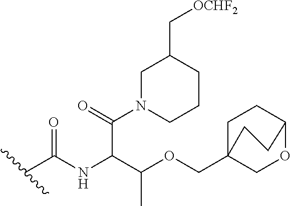 [SMILES: *C(=O)NC(C(=O)N1CCCC(COC(F)F)C1)C(C)OCC12CCC(CC1)OC2]

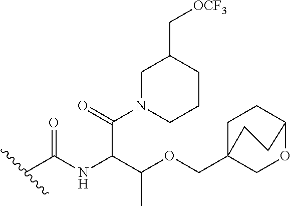 [SMILES: *C(=O)NC(C(=O)N1CCCC(COC(F)(F)F)C1)C(C)OCC12CCC(CC1)OC2]

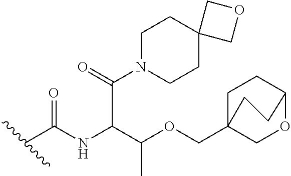 [SMILES: *C(=O)NC(C(=O)N1CCC2(CC1)COC2)C(C)OCC12CCC(CC1)OC2]

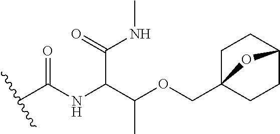 [SMILES: *C(=O)NC(C(=O)NC)C(C)OC[C@]12CC[C@H](CC1)O2]

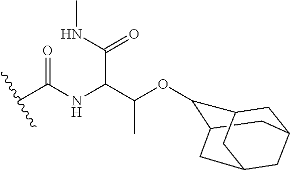 [SMILES: *C(=O)NC(C(=O)NC)C(C)OC1C2CC3CC(C2)CC1C3]

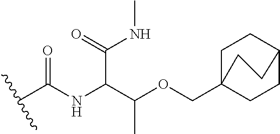 [SMILES: *C(=O)NC(C(=O)NC)C(C)OCC12CCC(CC1)CC2]

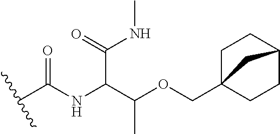 [SMILES: *C(=O)NC(C(=O)NC)C(C)OC[C@]12CC[C@H](CC1)C2]

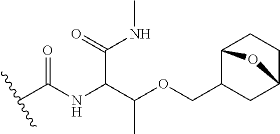 [SMILES: *C(=O)NC(C(=O)NC)C(C)OCC1C[C@H]2CC[C@@H]1O2]

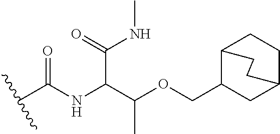 [SMILES: *C(=O)NC(C(=O)NC)C(C)OCC1CC2CCC1CC2]

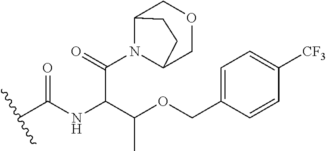 [SMILES: *C(=O)NC(C(=O)N1C2CCC1COC2)C(C)OCc1ccc(C(F)(F)F)cc1]

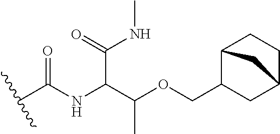 [SMILES: *C(=O)NC(C(=O)NC)C(C)OCC1C[C@H]2CC[C@@H]1C2]

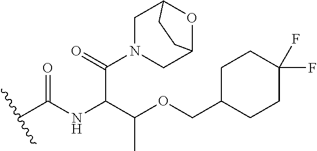 [SMILES: *C(=O)NC(C(=O)N1CC2CCC(C1)O2)C(C)OCC1CCC(F)(F)CC1]

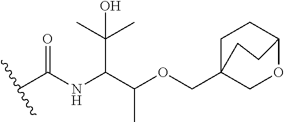 [SMILES: *C(=O)NC(C(C)OCC12CCC(CC1)OC2)C(C)(C)O]

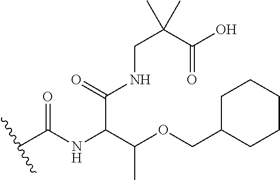 [SMILES: *C(=O)NC(C(=O)NCC(C)(C)C(=O)O)C(C)OCC1CCCCC1]

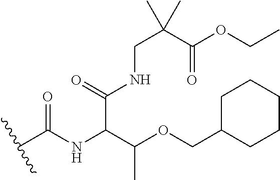 [SMILES: *C(=O)NC(C(=O)NCC(C)(C)C(=O)OCC)C(C)OCC1CCCCC1]

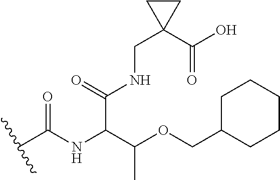 [SMILES: *C(=O)NC(C(=O)NCC1(C(=O)O)CC1)C(C)OCC1CCCCC1]

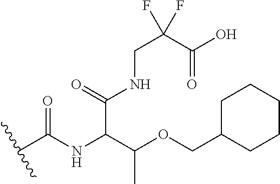 [SMILES: *C(=O)NC(C(=O)NCC(F)(F)C(=O)O)C(C)OCC1CCCCC1]

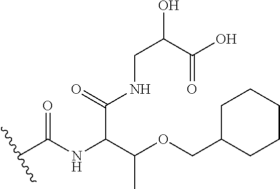 [SMILES: *C(=O)NC(C(=O)NCC(O)C(=O)O)C(C)OCC1CCCCC1]

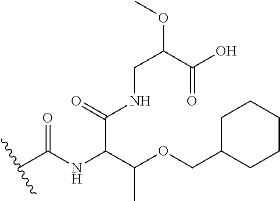 [SMILES: *C(=O)NC(C(=O)NCC(OC)C(=O)O)C(C)OCC1CCCCC1]

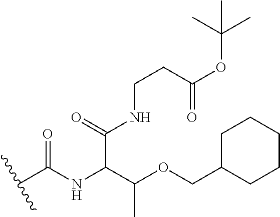 [SMILES: *C(=O)NC(C(=O)NCCC(=O)OC(C)(C)C)C(C)OCC1CCCCC1]

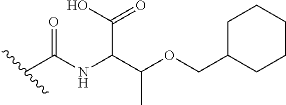 [SMILES: *C(=O)NC(C(=O)O)C(C)OCC1CCCCC1]

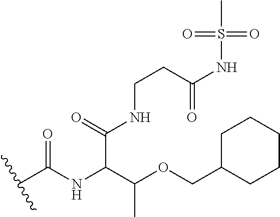 [SMILES: *C(=O)NC(C(=O)NCCC(=O)NS(C)(=O)=O)C(C)OCC1CCCCC1]

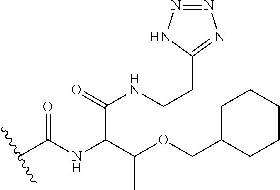 [SMILES: *C(=O)NC(C(=O)NCCc1nnn[nH]1)C(C)OCC1CCCCC1]

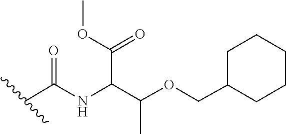 [SMILES: *C(=O)NC(C(=O)OC)C(C)OCC1CCCCC1]

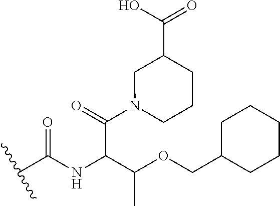 [SMILES: *C(=O)NC(C(=O)N1CCCC(C(=O)O)C1)C(C)OCC1CCCCC1]

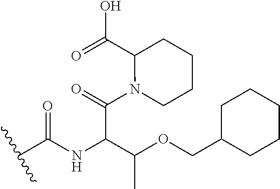 [SMILES: *C(=O)NC(C(=O)N1CCCCC1C(=O)O)C(C)OCC1CCCCC1]

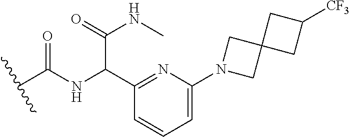 [SMILES: *C(=O)NC(C(=O)NC)c1cccc(N2CC3(CC(C(F)(F)F)C3)C2)n1]

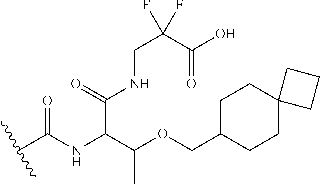 [SMILES: *C(=O)NC(C(=O)NCC(F)(F)C(=O)O)C(C)OCC1CCC2(CCC2)CC1]

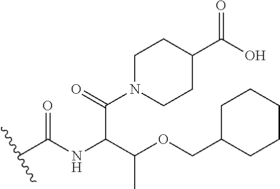 [SMILES: *C(=O)NC(C(=O)N1CCC(C(=O)O)CC1)C(C)OCC1CCCCC1]

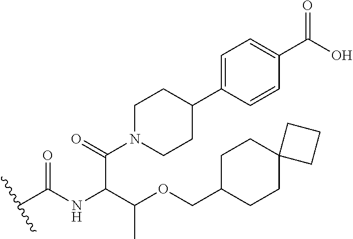 [SMILES: *C(=O)NC(C(=O)N1CCC(c2ccc(C(=O)O)cc2)CC1)C(C)OCC1CCC2(CCC2)CC1]

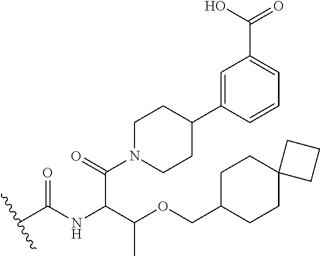 [SMILES: *C(=O)NC(C(=O)N1CCC(c2cccc(C(=O)O)c2)CC1)C(C)OCC1CCC2(CCC2)CC1]

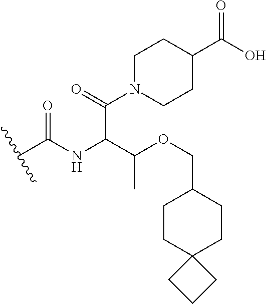 [SMILES: *C(=O)NC(C(=O)N1CCC(C(=O)O)CC1)C(C)OCC1CCC2(CCC2)CC1]

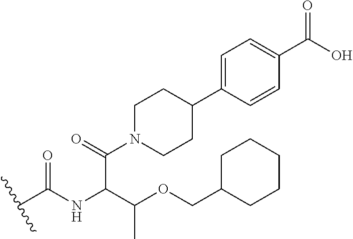 [SMILES: *C(=O)NC(C(=O)N1CCC(c2ccc(C(=O)O)cc2)CC1)C(C)OCC1CCCCC1]

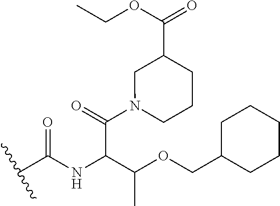 [SMILES: *C(=O)NC(C(=O)N1CCCC(C(=O)OCC)C1)C(C)OCC1CCCCC1]

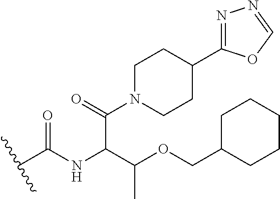 [SMILES: *C(=O)NC(C(=O)N1CCC(c2nnco2)CC1)C(C)OCC1CCCCC1]

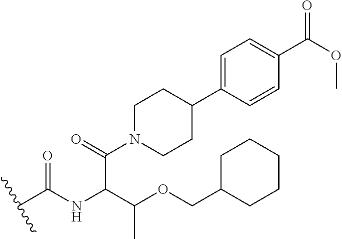 [SMILES: *C(=O)NC(C(=O)N1CCC(c2ccc(C(=O)OC)cc2)CC1)C(C)OCC1CCCCC1]

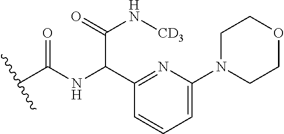 [SMILES: *C(=O)NC(C(=O)NC([2H])([2H])[2H])c1cccc(N2CCOCC2)n1]

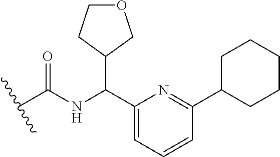 [SMILES: *C(=O)NC(c1cccc(C2CCCCC2)n1)C1CCOC1]

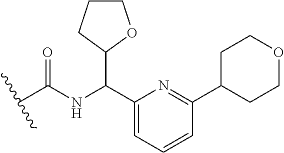 [SMILES: *C(=O)NC(c1cccc(C2CCOCC2)n1)C1CCCO1]

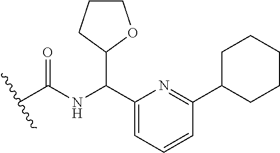 [SMILES: *C(=O)NC(c1cccc(C2CCCCC2)n1)C1CCCO1]

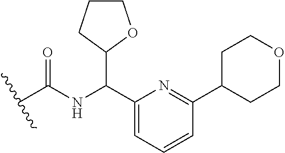 [SMILES: *C(=O)NC(c1cccc(C2CCOCC2)n1)C1CCCO1]

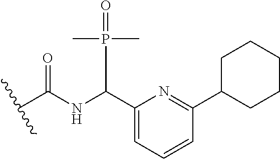 [SMILES: *C(=O)NC(c1cccc(C2CCCCC2)n1)P(C)(C)=O]

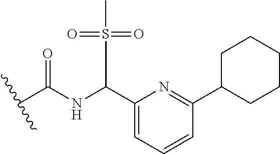 [SMILES: *C(=O)NC(c1cccc(C2CCCCC2)n1)S(C)(=O)=O]

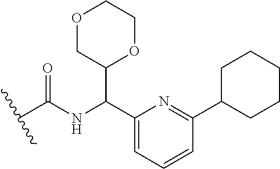 [SMILES: *C(=O)NC(c1cccc(C2CCCCC2)n1)C1COCCO1]

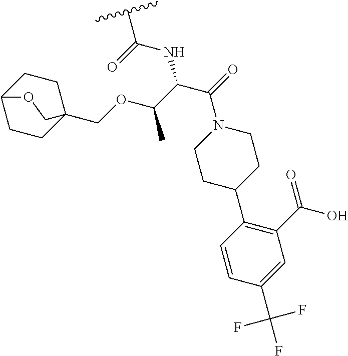 [SMILES: *C(=O)N[C@H](C(=O)N1CCC(c2ccc(C(F)(F)F)cc2C(=O)O)CC1)[C@@H](C)OCC12CCC(CC1)OC2]

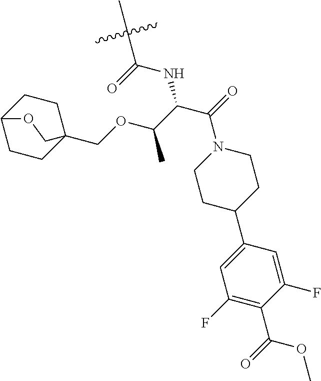 [SMILES: *C(=O)N[C@H](C(=O)N1CCC(c2cc(F)c(C(=O)OC)c(F)c2)CC1)[C@@H](C)OCC12CCC(CC1)OC2]

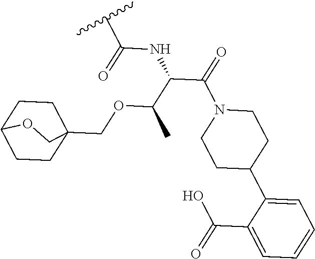 [SMILES: *C(=O)N[C@H](C(=O)N1CCC(c2ccccc2C(=O)O)CC1)[C@@H](C)OCC12CCC(CC1)OC2]

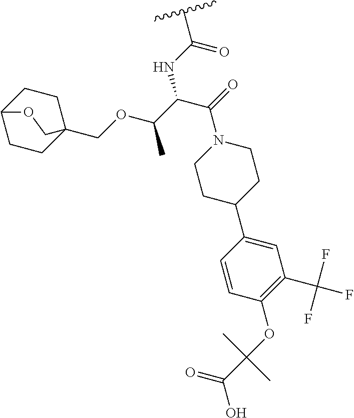 [SMILES: *C(=O)N[C@H](C(=O)N1CCC(c2ccc(OC(C)(C)C(=O)O)c(C(F)(F)F)c2)CC1)[C@@H](C)OCC12CCC(CC1)OC2]

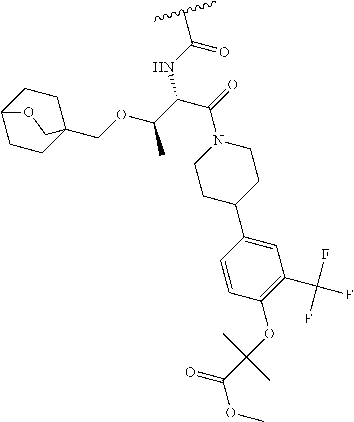 [SMILES: *C(=O)N[C@H](C(=O)N1CCC(c2ccc(OC(C)(C)C(=O)OC)c(C(F)(F)F)c2)CC1)[C@@H](C)OCC12CCC(CC1)OC2]

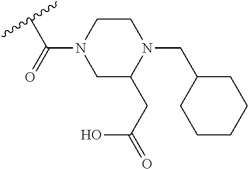 [SMILES: *C(=O)N1CCN(CC2CCCCC2)C(CC(=O)O)C1]

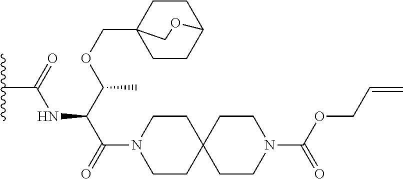 [SMILES: *C(=O)N[C@H](C(=O)N1CCC2(CCN(C(=O)OCC=C)CC2)CC1)[C@@H](C)OCC12CCC(CC1)OC2]

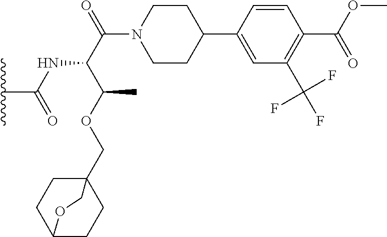 [SMILES: *C(=O)N[C@H](C(=O)N1CCC(c2ccc(C(=O)OC)c(C(F)(F)F)c2)CC1)[C@@H](C)OCC12CCC(CC1)OC2]

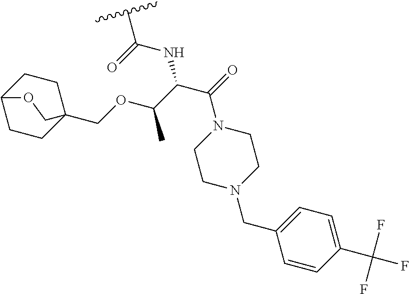 [SMILES: *C(=O)N[C@H](C(=O)N1CCN(Cc2ccc(C(F)(F)F)cc2)CC1)[C@@H](C)OCC12CCC(CC1)OC2]

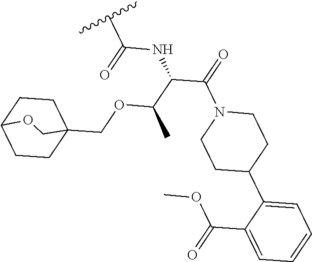 [SMILES: *C(=O)N[C@H](C(=O)N1CCC(c2ccccc2C(=O)OC)CC1)[C@@H](C)OCC12CCC(CC1)OC2]

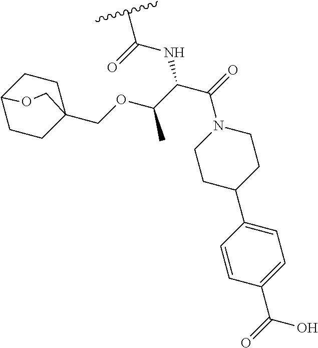 [SMILES: *C(=O)N[C@H](C(=O)N1CCC(c2ccc(C(=O)O)cc2)CC1)[C@@H](C)OCC12CCC(CC1)OC2]

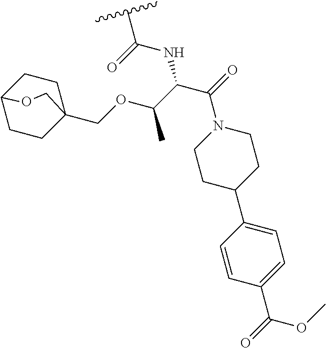 [SMILES: *C(=O)N[C@H](C(=O)N1CCC(c2ccc(C(=O)OC)cc2)CC1)[C@@H](C)OCC12CCC(CC1)OC2]

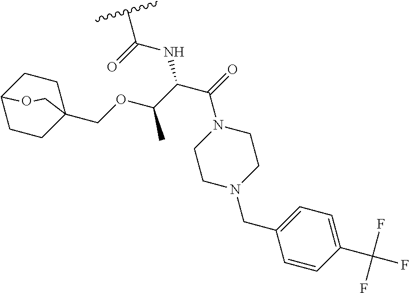 [SMILES: *C(=O)N[C@H](C(=O)N1CCN(Cc2ccc(C(F)(F)F)cc2)CC1)[C@@H](C)OCC12CCC(CC1)OC2]

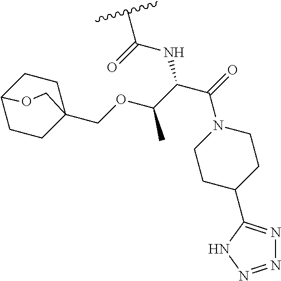 [SMILES: *C(=O)N[C@H](C(=O)N1CCC(c2nnn[nH]2)CC1)[C@@H](C)OCC12CCC(CC1)OC2]

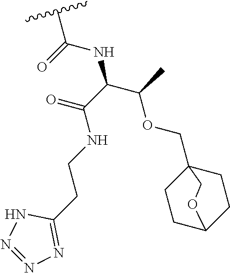 [SMILES: *C(=O)N[C@H](C(=O)NCCc1nnn[nH]1)[C@@H](C)OCC12CCC(CC1)OC2]

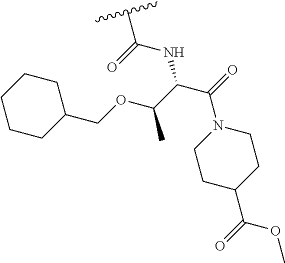 [SMILES: *C(=O)N[C@H](C(=O)N1CCC(C(=O)OC)CC1)[C@@H](C)OCC1CCCCC1]

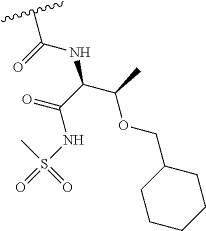 [SMILES: *C(=O)N[C@H](C(=O)NS(C)(=O)=O)[C@@H](C)OCC1CCCCC1]

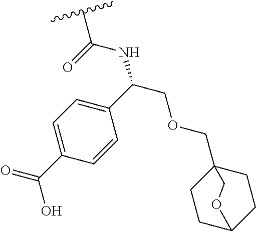 [SMILES: *C(=O)N[C@H](COCC12CCC(CC1)OC2)c1ccc(C(=O)O)cc1]

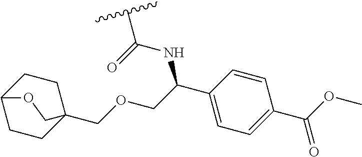 [SMILES: *C(=O)N[C@H](COCC12CCC(CC1)OC2)c1ccc(C(=O)OC)cc1]

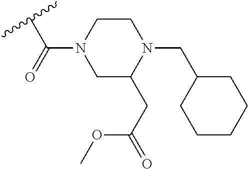 [SMILES: *C(=O)N1CCN(CC2CCCCC2)C(CC(=O)OC)C1]

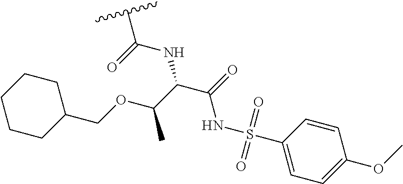 [SMILES: *C(=O)N[C@H](C(=O)NS(=O)(=O)c1ccc(OC)cc1)[C@@H](C)OCC1CCCCC1]

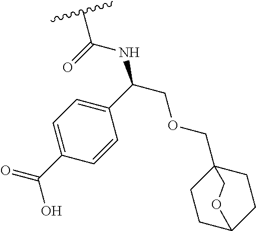 [SMILES: *C(=O)N[C@@H](COCC12CCC(CC1)OC2)c1ccc(C(=O)O)cc1]

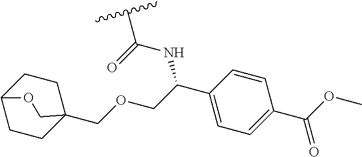 [SMILES: *C(=O)N[C@@H](COCC12CCC(CC1)OC2)c1ccc(C(=O)OC)cc1]

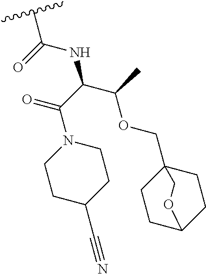 [SMILES: *C(=O)N[C@H](C(=O)N1CCC(C#N)CC1)[C@@H](C)OCC12CCC(CC1)OC2]

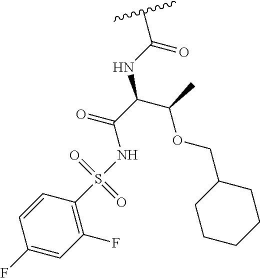 [SMILES: *C(=O)N[C@H](C(=O)NS(=O)(=O)c1ccc(F)cc1F)[C@@H](C)OCC1CCCCC1]

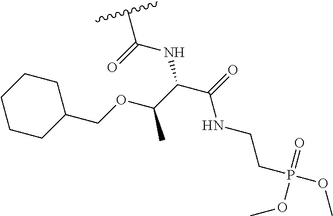 [SMILES: *C(=O)N[C@H](C(=O)NCCP(=O)(OC)OC)[C@@H](C)OCC1CCCCC1]

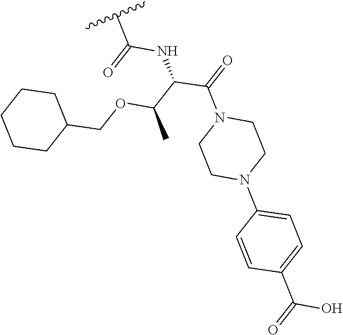 [SMILES: *C(=O)N[C@H](C(=O)N1CCN(c2ccc(C(=O)O)cc2)CC1)[C@@H](C)OCC1CCCCC1]

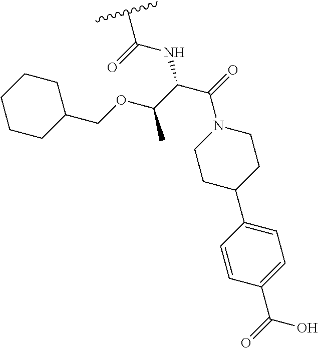 [SMILES: *C(=O)N[C@H](C(=O)N1CCC(c2ccc(C(=O)O)cc2)CC1)[C@@H](C)OCC1CCCCC1]

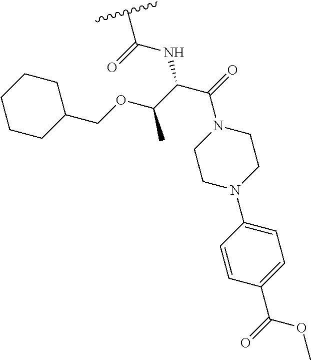 [SMILES: *C(=O)N[C@H](C(=O)N1CCN(c2ccc(C(=O)OC)cc2)CC1)[C@@H](C)OCC1CCCCC1]

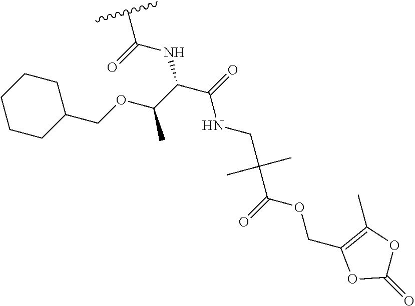 [SMILES: *C(=O)N[C@H](C(=O)NCC(C)(C)C(=O)OCc1oc(=O)oc1C)[C@@H](C)OCC1CCCCC1]

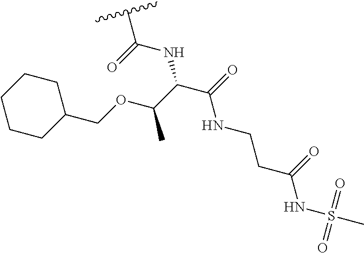 [SMILES: *C(=O)N[C@H](C(=O)NCCC(=O)NS(C)(=O)=O)[C@@H](C)OCC1CCCCC1]

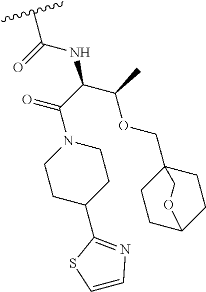 [SMILES: *C(=O)N[C@H](C(=O)N1CCC(c2nccs2)CC1)[C@@H](C)OCC12CCC(CC1)OC2]

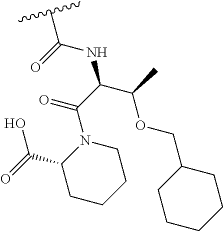 [SMILES: *C(=O)N[C@H](C(=O)N1CCCC[C@@H]1C(=O)O)[C@@H](C)OCC1CCCCC1]

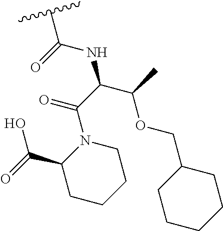 [SMILES: *C(=O)N[C@H](C(=O)N1CCCC[C@H]1C(=O)O)[C@@H](C)OCC1CCCCC1]

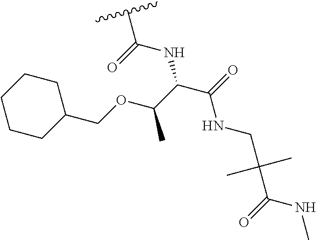 [SMILES: *C(=O)N[C@H](C(=O)NCC(C)(C)C(=O)NC)[C@@H](C)OCC1CCCCC1]

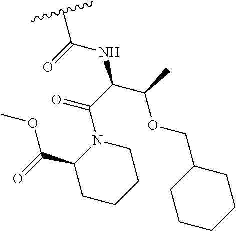 [SMILES: *C(=O)N[C@H](C(=O)N1CCCC[C@H]1C(=O)OC)[C@@H](C)OCC1CCCCC1]

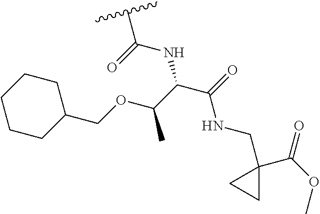 [SMILES: *C(=O)N[C@H](C(=O)NCC1(C(=O)OC)CC1)[C@@H](C)OCC1CCCCC1]

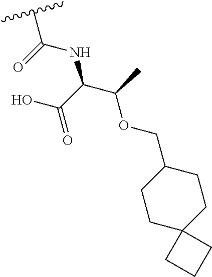 [SMILES: *C(=O)N[C@H](C(=O)O)[C@@H](C)OCC1CCC2(CCC2)CC1]

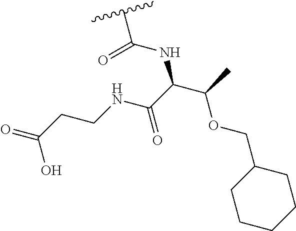 [SMILES: *C(=O)N[C@H](C(=O)NCCC(=O)O)[C@@H](C)OCC1CCCCC1]

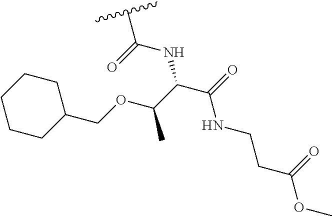 [SMILES: *C(=O)N[C@H](C(=O)NCCC(=O)OC)[C@@H](C)OCC1CCCCC1]

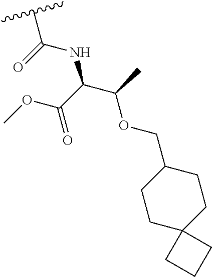 [SMILES: *C(=O)N[C@H](C(=O)OC)[C@@H](C)OCC1CCC2(CCC2)CC1]

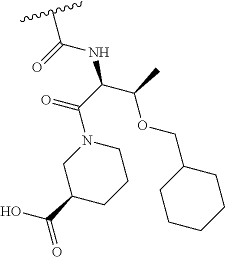 [SMILES: *C(=O)N[C@H](C(=O)N1CCC[C@@H](C(=O)O)C1)[C@@H](C)OCC1CCCCC1]

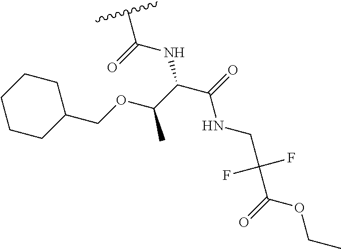 [SMILES: *C(=O)N[C@H](C(=O)NCC(F)(F)C(=O)OCC)[C@@H](C)OCC1CCCCC1]

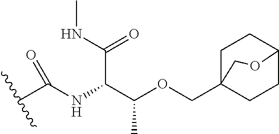 [SMILES: *C(=O)N[C@H](C(=O)NC)[C@@H](C)OCC12CCC(CC1)OC2]

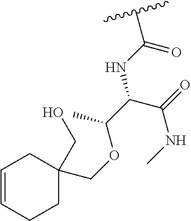 [SMILES: *C(=O)N[C@H](C(=O)NC)[C@@H](C)OCC1(CO)CC=CCC1]

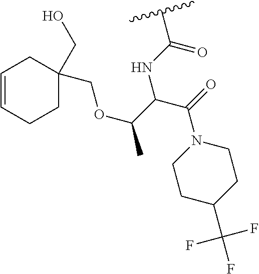 [SMILES: *C(=O)NC(C(=O)N1CCC(C(F)(F)F)CC1)[C@@H](C)OCC1(CO)CC=CCC1]

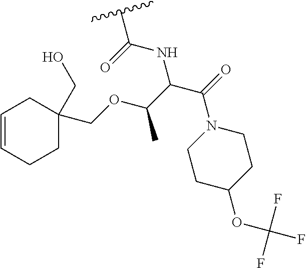 [SMILES: *C(=O)NC(C(=O)N1CCC(OC(F)(F)F)CC1)[C@@H](C)OCC1(CO)CC=CCC1]

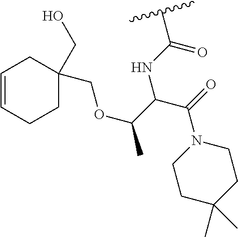 [SMILES: *C(=O)NC(C(=O)N1CCC(C)(C)CC1)[C@@H](C)OCC1(CO)CC=CCC1]

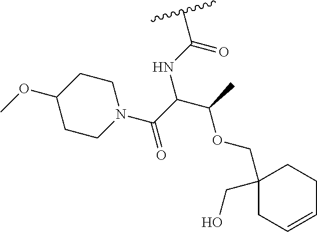 [SMILES: *C(=O)NC(C(=O)N1CCC(OC)CC1)[C@@H](C)OCC1(CO)CC=CCC1]

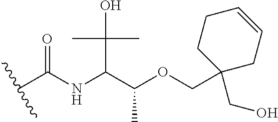 [SMILES: *C(=O)NC([C@@H](C)OCC1(CO)CC=CCC1)C(C)(C)O]

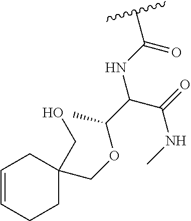 [SMILES: *C(=O)NC(C(=O)NC)[C@@H](C)OCC1(CO)CC=CCC1]

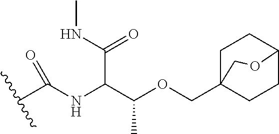 [SMILES: *C(=O)NC(C(=O)NC)[C@@H](C)OCC12CCC(CC1)OC2]

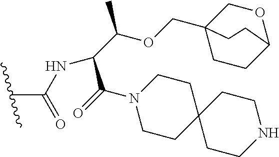 [SMILES: *C(=O)N[C@H](C(=O)N1CCC2(CCNCC2)CC1)[C@@H](C)OCC12CCC(CC1)OC2]

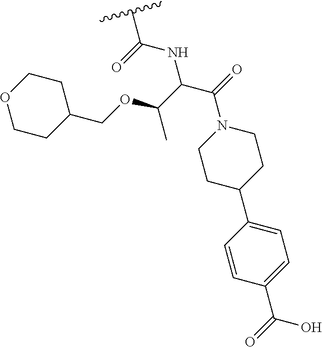 [SMILES: *C(=O)NC(C(=O)N1CCC(c2ccc(C(=O)O)cc2)CC1)[C@@H](C)OCC1CCOCC1]

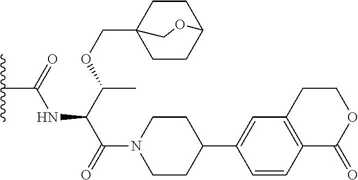 [SMILES: *C(=O)N[C@H](C(=O)N1CCC(c2ccc3c(c2)CCOC3=O)CC1)[C@@H](C)OCC12CCC(CC1)OC2]

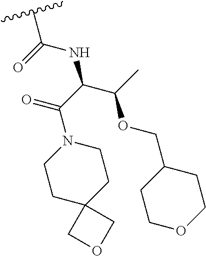 [SMILES: *C(=O)N[C@H](C(=O)N1CCC2(CC1)COC2)[C@@H](C)OCC1CCOCC1]

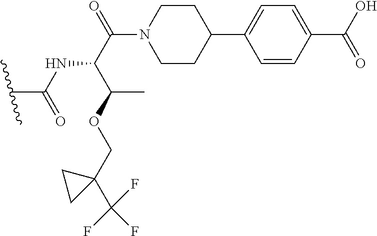 [SMILES: *C(=O)N[C@H](C(=O)N1CCC(c2ccc(C(=O)O)cc2)CC1)[C@@H](C)OCC1(C(F)(F)F)CC1]

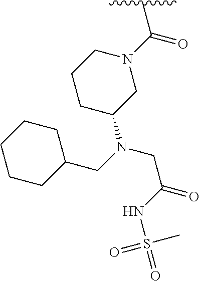 [SMILES: *C(=O)N1CCC[C@@H](N(CC(=O)NS(C)(=O)=O)CC2CCCCC2)C1]

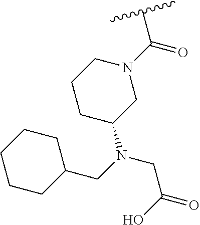 [SMILES: *C(=O)N1CCC[C@@H](N(CC(=O)O)CC2CCCCC2)C1]

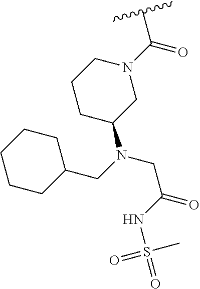 [SMILES: *C(=O)N1CCC[C@H](N(CC(=O)NS(C)(=O)=O)CC2CCCCC2)C1]

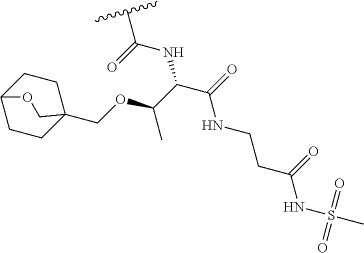 [SMILES: *C(=O)N[C@H](C(=O)NCCC(=O)NS(C)(=O)=O)[C@@H](C)OCC12CCC(CC1)OC2]

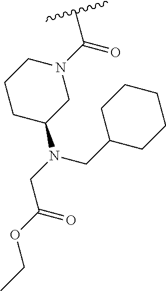 [SMILES: *C(=O)N1CCC[C@H](N(CC(=O)OCC)CC2CCCCC2)C1]

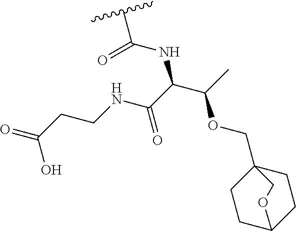 [SMILES: *C(=O)N[C@H](C(=O)NCCC(=O)O)[C@@H](C)OCC12CCC(CC1)OC2]

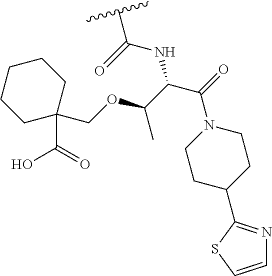 [SMILES: *C(=O)N[C@H](C(=O)N1CCC(c2nccs2)CC1)[C@@H](C)OCC1(C(=O)O)CCCCC1]

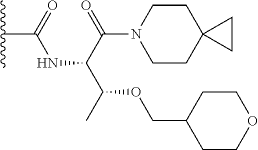 [SMILES: *C(=O)N[C@H](C(=O)N1CCC2(CC1)CC2)[C@@H](C)OCC1CCOCC1]

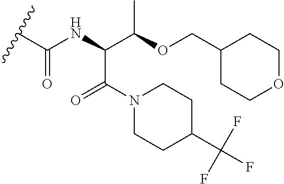 [SMILES: *C(=O)N[C@H](C(=O)N1CCC(C(F)(F)F)CC1)[C@@H](C)OCC1CCOCC1]

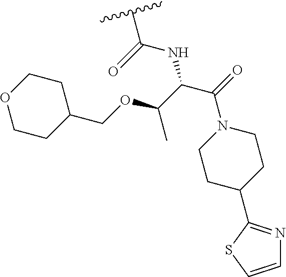 [SMILES: *C(=O)N[C@H](C(=O)N1CCC(c2nccs2)CC1)[C@@H](C)OCC1CCOCC1]

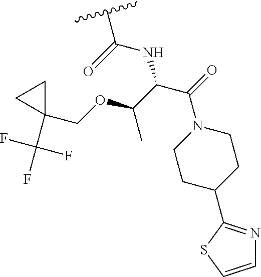 [SMILES: *C(=O)N[C@H](C(=O)N1CCC(c2nccs2)CC1)[C@@H](C)OCC1(C(F)(F)F)CC1]

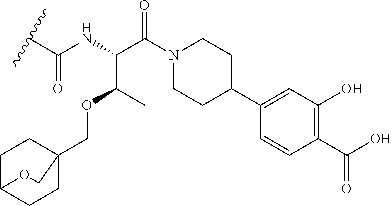 [SMILES: *C(=O)N[C@H](C(=O)N1CCC(c2ccc(C(=O)O)c(O)c2)CC1)[C@@H](C)OCC12CCC(CC1)OC2]

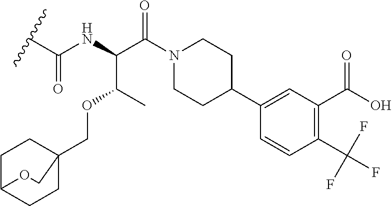 [SMILES: *C(=O)N[C@@H](C(=O)N1CCC(c2ccc(C(F)(F)F)c(C(=O)O)c2)CC1)[C@H](C)OCC12CCC(CC1)OC2]

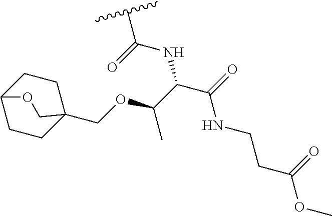 [SMILES: *C(=O)N[C@H](C(=O)NCCC(=O)OC)[C@@H](C)OCC12CCC(CC1)OC2]

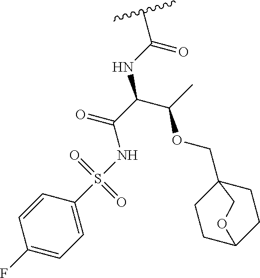 [SMILES: *C(=O)N[C@H](C(=O)NS(=O)(=O)c1ccc(F)cc1)[C@@H](C)OCC12CCC(CC1)OC2]

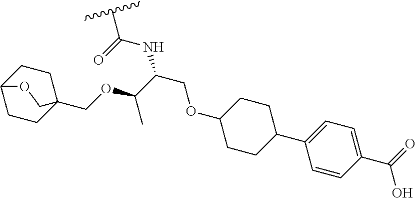 [SMILES: *C(=O)N[C@H](COC1CCC(c2ccc(C(=O)O)cc2)CC1)[C@@H](C)OCC12CCC(CC1)OC2]

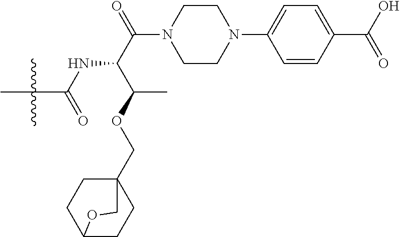 [SMILES: *C(=O)N[C@H](C(=O)N1CCN(c2ccc(C(=O)O)cc2)CC1)[C@@H](C)OCC12CCC(CC1)OC2]

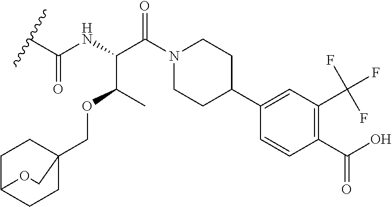 [SMILES: *C(=O)N[C@H](C(=O)N1CCC(c2ccc(C(=O)O)c(C(F)(F)F)c2)CC1)[C@@H](C)OCC12CCC(CC1)OC2]

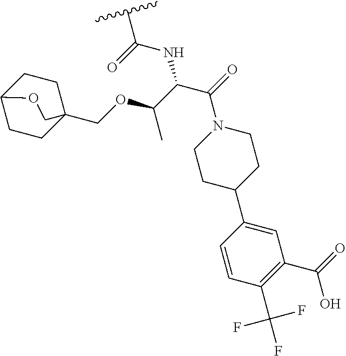 [SMILES: *C(=O)N[C@H](C(=O)N1CCC(c2ccc(C(F)(F)F)c(C(=O)O)c2)CC1)[C@@H](C)OCC12CCC(CC1)OC2]

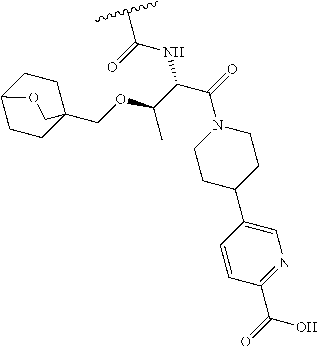 [SMILES: *C(=O)N[C@H](C(=O)N1CCC(c2ccc(C(=O)O)nc2)CC1)[C@@H](C)OCC12CCC(CC1)OC2]

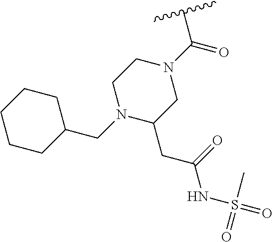 [SMILES: *C(=O)N1CCN(CC2CCCCC2)C(CC(=O)NS(C)(=O)=O)C1]

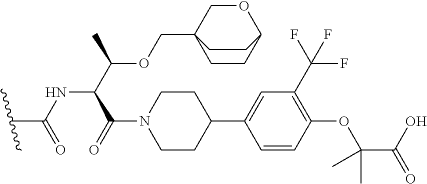 [SMILES: *C(=O)N[C@H](C(=O)N1CCC(c2ccc(OC(C)(C)C(=O)O)c(C(F)(F)F)c2)CC1)[C@@H](C)OCC12CCC(CC1)OC2]

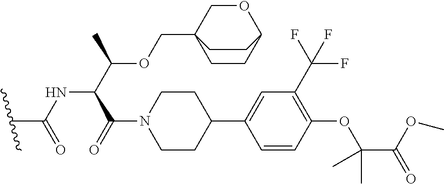 [SMILES: *C(=O)N[C@H](C(=O)N1CCC(c2ccc(OC(C)(C)C(=O)OC)c(C(F)(F)F)c2)CC1)[C@@H](C)OCC12CCC(CC1)OC2]

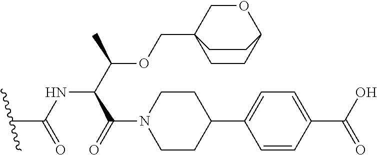 [SMILES: *C(=O)N[C@H](C(=O)N1CCC(c2ccc(C(=O)O)cc2)CC1)[C@@H](C)OCC12CCC(CC1)OC2]

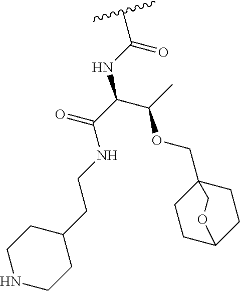 [SMILES: *C(=O)N[C@H](C(=O)NCCC1CCNCC1)[C@@H](C)OCC12CCC(CC1)OC2]

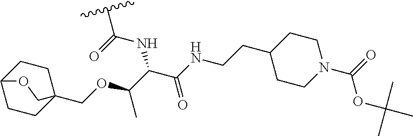 [SMILES: *C(=O)N[C@H](C(=O)NCCC1CCN(C(=O)OC(C)(C)C)CC1)[C@@H](C)OCC12CCC(CC1)OC2]

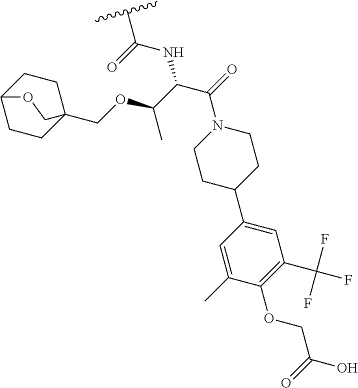 [SMILES: *C(=O)N[C@H](C(=O)N1CCC(c2cc(C)c(OCC(=O)O)c(C(F)(F)F)c2)CC1)[C@@H](C)OCC12CCC(CC1)OC2]

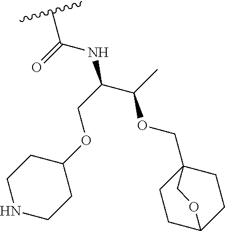 [SMILES: *C(=O)N[C@H](COC1CCNCC1)[C@@H](C)OCC12CCC(CC1)OC2]

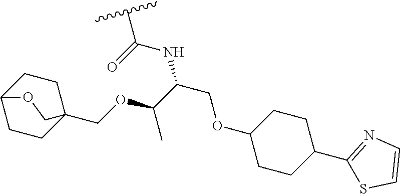 [SMILES: *C(=O)N[C@H](COC1CCC(c2nccs2)CC1)[C@@H](C)OCC12CCC(CC1)OC2]

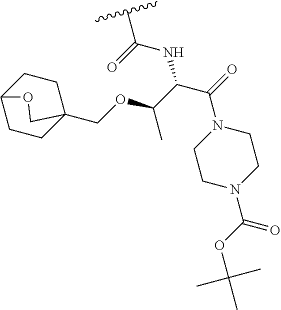 [SMILES: *C(=O)N[C@H](C(=O)N1CCN(C(=O)OC(C)(C)C)CC1)[C@@H](C)OCC12CCC(CC1)OC2]

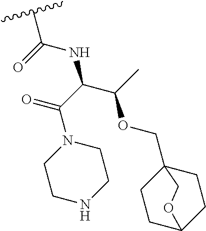 [SMILES: *C(=O)N[C@H](C(=O)N1CCNCC1)[C@@H](C)OCC12CCC(CC1)OC2]

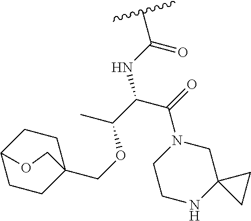 [SMILES: *C(=O)N[C@H](C(=O)N1CCNC2(CC2)C1)[C@@H](C)OCC12CCC(CC1)OC2]

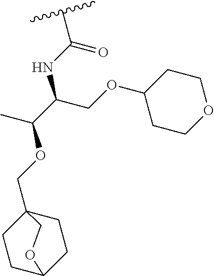 [SMILES: *C(=O)N[C@@H](COC1CCOCC1)[C@H](C)OCC12CCC(CC1)OC2]

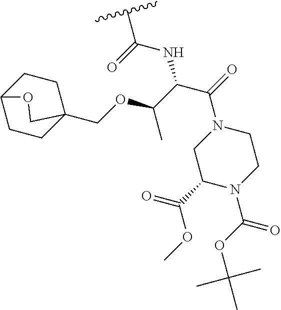 [SMILES: *C(=O)N[C@H](C(=O)N1CCN(C(=O)OC(C)(C)C)[C@H](C(=O)OC)C1)[C@@H](C)OCC12CCC(CC1)OC2]

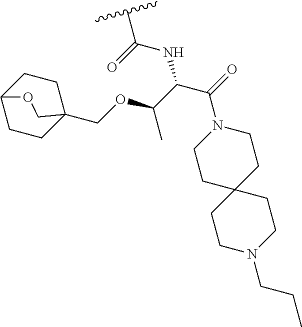 [SMILES: *C(=O)N[C@H](C(=O)N1CCC2(CCN(CCC)CC2)CC1)[C@@H](C)OCC12CCC(CC1)OC2]

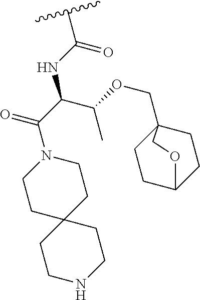 [SMILES: *C(=O)N[C@H](C(=O)N1CCC2(CCNCC2)CC1)[C@@H](C)OCC12CCC(CC1)OC2]

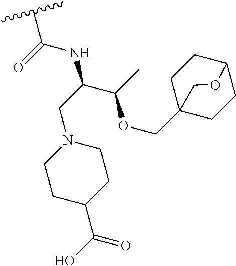 [SMILES: *C(=O)N[C@H](CN1CCC(C(=O)O)CC1)[C@@H](C)OCC12CCC(CC1)OC2]

In some embodiments, R^{A} is

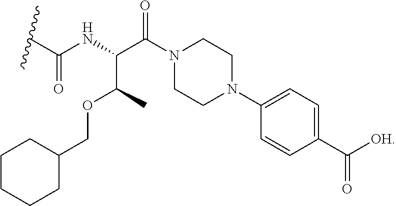 [SMILES: *C(=O)N[C@H](C(=O)N1CCN(c2ccc(C(=O)O)cc2)CC1)[C@@H](C)OCC1CCCCC1]

In some embodiments, R^{A} is

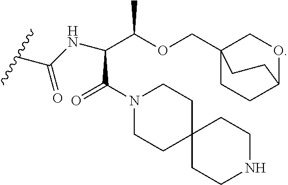 [SMILES: *C(=O)N[C@H](C(=O)N1CCC2(CCNCC2)CC1)[C@@H](C)OCC12CCC(CC1)OC2]

As defined generally above, L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L² is a saturated or unsaturated, straight or branched, optionally substituted bivalent C₁₋₄ hydrocarbon chain, wherein 0-2 methylene units of L² are independently replaced by —C(O)O—, —C(O)—, or —C(O)NR—. In some embodiments, L² is a C₁₋₄ alkylene chain, wherein 1-2 methylene units of L² are independently replaced by —C(O)O—, —C(O)—, or —C(O)NR—. In some embodiments, L² is C₁₋₄ alkylene chain, wherein 1 methylene unit of L² is replaced by —C(O)O—, —C(O)—, or —C(O)NR—. In some embodiments, L² is a saturated optionally substituted bivalent C₁₋₄ hydrocarbon chain. In some embodiments, L² is a saturated bivalent C₁₋₄ hydrocarbon chain, substituted on a single methylene unit by two substituents, which together with the intervening carbon atom form a 3-7 membered carbocyclic ring or heterocyclic ring (having 1-2 heteroatoms independently selected from nitrogen, oxygen, and sulfur). In some embodiments, L²

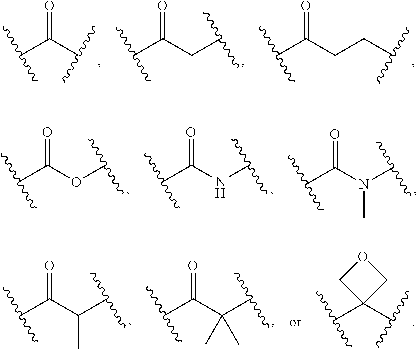

In some embodiments, L² is

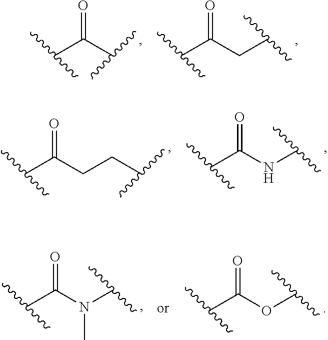

In some embodiments, L² is

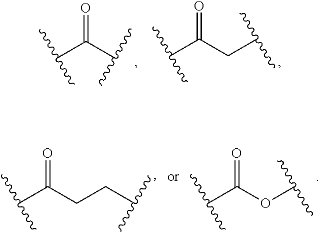

In some embodiments, L² is

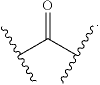 [SMILES: *C(*)=O]

In some embodiments, L² is selected from those depicted in the compounds of Table 1, below.

In some embodiments, L² is a saturated, straight or branched, optionally substituted bivalent C₁₋₄ hydrocarbon chain. In some embodiments, L² is methylene.

In some embodiments, L² is —S(O)₂—.

As defined generally above, R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷.

In some embodiments, R⁶ is an optionally substituted C₁₋₆ aliphatic group. In some embodiments, R⁶ is an optionally substituted methyl, ethyl, isopropyl, or tert-butyl group.

In some embodiments, R⁶ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷. In some embodiments, R⁶ is a 3-8 membered saturated or partially unsaturated monocyclic carbocyclic ring, optionally substituted with one or more instances of R⁷. In some embodiments, R⁶ is a phenyl group, optionally substituted with one or more instances of R⁷. In some embodiments, R⁶ is a cyclic group selected from cyclopropyl, cyclobutyl, cyclohexyl and phenyl, wherein the cyclic group is optionally substituted with one or more instances of R⁷. In some embodiments, R⁶ is a cyclopropyl group, optionally substituted with one or more instances of R⁷. In some embodiments, R⁶ is a cyclopropyl group, optionally substituted with one instance of —CF₃. In some embodiments, R⁶ is selected from those depicted in the compounds of Table 1, below.

In some embodiments, R⁶ is a 3-8 membered saturated or partially unsaturated monocyclic heterocyclic ring (having 1-2 heteroatoms independently selected from nitrogen, oxygen, and sulfur), optionally substituted with one or more instances of R⁷. In some embodiments, R⁶ is tetrahydrofuranyl, optionally substituted with one or more instances of R⁷. In some embodiments, R⁶ is tetrahydropyranyl, optionally substituted with one or more instances of R⁷. In some embodiments, R⁶ is oxetanyl, optionally substituted with one or more instances of R⁷.

In some embodiments, R⁶ is a 5-6 membered monocyclic heteroaromatic ring (having 1-4 heteroatoms independently selected from nitrogen, oxygen, and sulfur), optionally substituted with one or more instances of R⁷. In some embodiments, R⁶ is furanyl, optionally substituted with one or more instances of R⁷. In some embodiments, R⁶ is pyrazolyl, optionally substituted with one or more instances of R⁷. In some embodiments, R⁶ is oxazolyl, optionally substituted with one or more instances of R⁷.

As defined generally above, each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy. In some embodiments, each instance of R⁷ is independently halogen, —OR, —CN, an optionally substituted C₁₋₆ aliphatic group, an optionally substituted C₁₋₆ aliphatic-Cy group, or Cy. In some embodiments, each instance of R⁷ is independently —F, methyl, ethyl, isopropyl, isobutyl, —CN, optionally substituted phenyl, optionally substituted benzyl, —CF₃, —CH₂OH, —CH₂OCH₃, —CH₂CH₂OCH₃, —CH₂CH₂F, cyclopropyl or —CH₂-(cyclopropyl). In some embodiments, each instance of R⁷ is independently a C₁₋₆ aliphatic group. In some embodiments, R⁷ is —CF₃.

In some embodiments, -L²-R⁶ is a substituent of Table C:

TABLE C
Exemplary-L²-R⁶ substituents

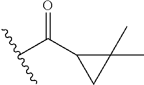 [SMILES: *C(=O)C1CC1(C)C]

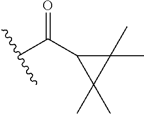 [SMILES: *C(=O)C1C(C)(C)C1(C)C]

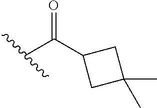 [SMILES: *C(=O)C1CC(C)(C)C1]

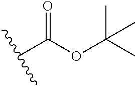 [SMILES: *C(=O)OC(C)(C)C]

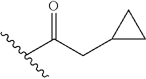 [SMILES: *C(=O)CC1CC1]

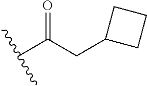 [SMILES: *C(=O)CC1CCC1]

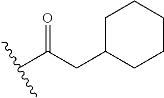 [SMILES: *C(=O)CC1CCCCC1]

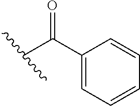 [SMILES: *C(=O)c1ccccc1]

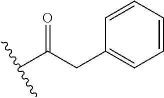 [SMILES: *C(=O)Cc1ccccc1]

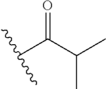 [SMILES: *C(=O)C(C)C]

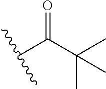 [SMILES: *C(=O)C(C)(C)C]

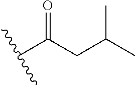 [SMILES: *C(=O)CC(C)C]

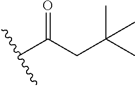 [SMILES: *C(=O)CC(C)(C)C]

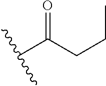 [SMILES: *C(=O)CCC]

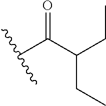 [SMILES: *C(=O)C(CC)CC]

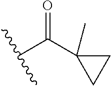 [SMILES: *C(=O)C1(C)CC1]

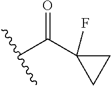 [SMILES: *C(=O)C1(F)CC1]

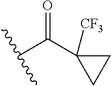 [SMILES: *C(=O)C1(C(F)(F)F)CC1]

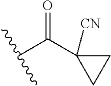 [SMILES: *C(=O)C1(C#N)CC1]

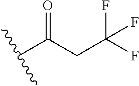 [SMILES: *C(=O)CC(F)(F)F]

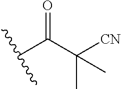 [SMILES: *C(=O)C(C)(C)C#N]

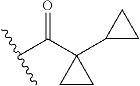 [SMILES: *C(=O)C1(C2CC2)CC1]

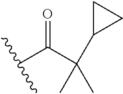 [SMILES: *C(=O)C(C)(C)C1CC1]

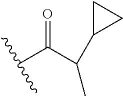 [SMILES: *C(=O)C(C)C1CC1]

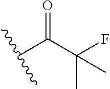 [SMILES: *C(=O)C(C)(C)F]

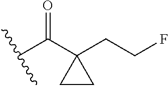 [SMILES: *C(=O)C1(CCF)CC1]

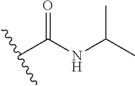 [SMILES: *C(=O)NC(C)C]

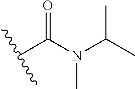 [SMILES: *C(=O)N(C)C(C)C]

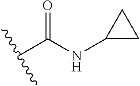 [SMILES: *C(=O)NC1CC1]

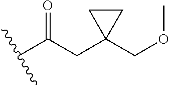 [SMILES: *C(=O)CC1(COC)CC1]

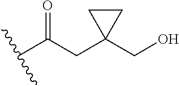 [SMILES: *C(=O)CC1(CO)CC1]

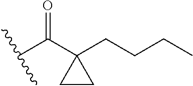 [SMILES: *C(=O)C1(CCCC)CC1]

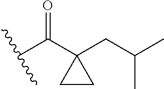 [SMILES: *C(=O)C1(CC(C)C)CC1]

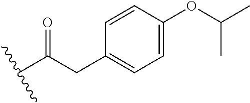 [SMILES: *C(=O)Cc1ccc(OC(C)C)cc1]

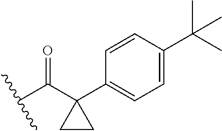 [SMILES: *C(=O)C1(c2ccc(C(C)(C)C)cc2)CC1]

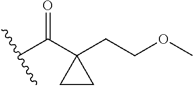 [SMILES: *C(=O)C1(CCOC)CC1]

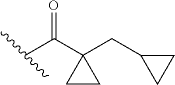 [SMILES: *C(=O)C1(CC2CC2)CC1]

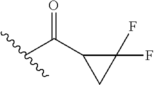 [SMILES: *C(=O)C1CC1(F)F]

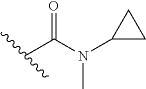 [SMILES: *C(=O)N(C)C1CC1]

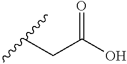 [SMILES: *CC(=O)O]

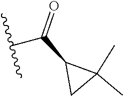 [SMILES: *C(=O)[C@@H]1CC1(C)C]

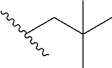 [SMILES: *CC(C)(C)C]

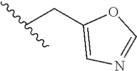 [SMILES: *Cc1cnco1]

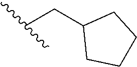 [SMILES: *CC1CCCC1]

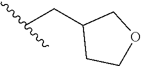 [SMILES: *CC1CCOC1]

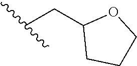 [SMILES: *CC1CCCO1]

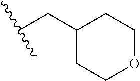 [SMILES: *CC1CCOCC1]

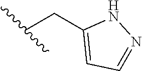 [SMILES: *Cc1ccn[nH]1]

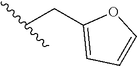 [SMILES: *Cc1ccco1]

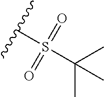 [SMILES: *S(=O)(=O)C(C)(C)C]

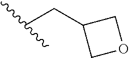 [SMILES: *CC1COC1]

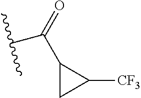 [SMILES: *C(=O)C1CC1C(F)(F)F]

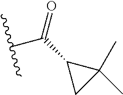 [SMILES: *C(=O)[C@H]1CC1(C)C]

In some embodiments, -L^{Z}-R⁶ is

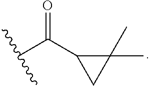 [SMILES: *C(=O)C1CC1(C)C]

In some embodiments, -L^{Z}-R⁶ is

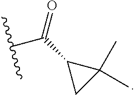 [SMILES: *C(=O)[C@H]1CC1(C)C]

In some embodiments, -L^{Z}-R⁶ is

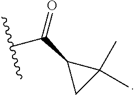 [SMILES: *C(=O)[C@@H]1CC1(C)C]

In some embodiments, -L²-R⁶ is

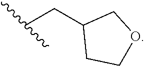 [SMILES: *CC1CCOC1]

In some embodiments, -L²-R⁶ is

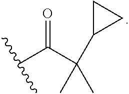 [SMILES: *C(=O)C(C)(C)C1CC1]

In some embodiments, -L²-R is

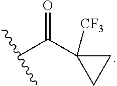 [SMILES: *C(=O)C1(C(F)(F)F)CC1]

As defined generally above, L³ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—.

In some embodiments, L³ is a saturated or unsaturated, straight or branched, optionally substituted bivalent C₁₋₄ hydrocarbon chain, wherein 0-2 methylene units of L³ are independently replaced by —S(O)₂—, —C(O)NR—, or —C(O)—. In some embodiments, L³ is a C₁₋₄ alkylene chain, wherein 1-2 methylene units of L³ are independently replaced by —S(O)₂—, —C(O)NR—, or —C(O)—. In some embodiments, L³ is C₁₋₄ alkylene chain, wherein 1 methylene unit of L³ is replaced by —S(O)₂—, —C(O)NR—, or —C(O)—. In some embodiments, L³ is a saturated or unsaturated, straight or branched, optionally substituted bivalent C₁₋₄ alkylene chain, wherein 0-2 methylene units of L³ are independently replaced by —C(O)O—, or —C(O)—. In some embodiments, L³ is a C₁₋₄ alkylene chain, wherein 1-2 methylene units of L³ are independently replaced by —C(O)O—, or —C(O)—. In some embodiments, L³ is C₁₋₄ alkylene chain, wherein 1 methylene unit of L³ is replaced by —C(O)O—, or —C(O)—. In some embodiments, L³ is a saturated optionally substituted bivalent C₁₋₄ hydrocarbon chain. In some embodiments, L³ is a saturated bivalent C₁₋₄ hydrocarbon chain, substituted on a single methylene unit by two substituents, which together with the intervening carbon atom form a 3-7 membered carbocyclic ring or heterocyclic ring (having 1-2 heteroatoms independently selected from nitrogen, oxygen, and sulfur). In some embodiments, L³ is

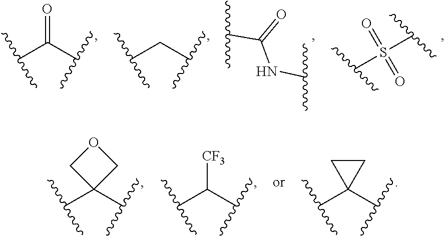

In some embodiments, L³ is

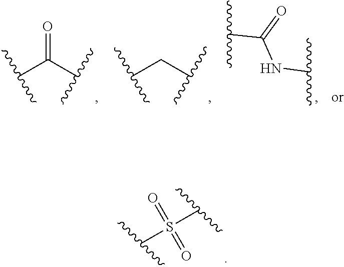

In some embodiments, L¹ is

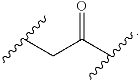 [SMILES: *CC(*)=O]

In some embodiments, L³ is

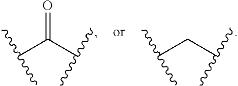

In some embodiments, L³ is

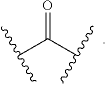 [SMILES: *C(*)=O]

In some embodiments, L³ is selected from those depicted in the compounds of Table 1, below.

As defined generally above, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹.

In some embodiments, R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a cyclic group selected from a 3-8 membered saturated or partially unsaturated monocyclic heterocyclic ring (having 1-2 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹. In some embodiments, R⁸ is a 3-8 membered saturated or partially unsaturated monocyclic heterocyclic ring (having 1-2 heteroatoms independently selected from nitrogen, oxygen, and sulfur), optionally substituted with one or more instances of R⁹. In some embodiments, R⁸ is a 5-6 membered monocyclic heteroaromatic ring (having 1-4 heteroatoms independently selected from nitrogen, oxygen, and sulfur), optionally substituted with one or more instances of R⁹. In some embodiments, R⁸ is an 8-10 membered bicyclic heteroaromatic ring (having 1-5 heteroatoms independently selected from nitrogen, oxygen, and sulfur), optionally substituted with one or more instances of R⁹. In some embodiments, R⁸ is a cyclic group selected from pyrazolyl, oxazolyl, thiazolyl, pyrrolidinyl, tetrahydropyranyl, pyridinyl, imidazolyl, indolyl, 1,2,4-triazolyl, 1,2,4-thiadiazolyl, piperidinyl, pyrazinyl, and indazolyl, wherein the cyclic group is optionally substituted with one or more instances of R⁹. In some embodiments, R⁸ is a pyrazolyl or thiazolyl group, optionally substituted with one or more instances of R⁹. In some embodiments, R⁸ is a pyrazolyl or thiazolyl group. In some embodiments, R⁸ is selected from those depicted in the compounds of Table 1, below.

As defined generally above, each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy.

In some embodiments, each instance of R⁹ is independently halogen, an optionally substituted C₁₋₆ aliphatic group, an optionally substituted C₁₋₆ aliphatic-Cy group, or Cy. In some embodiments, each instance of R⁹ is independently an optionally substituted C₁₋₆ aliphatic-Cy group, wherein the Cy is an optionally substituted group selected from phenyl, cyclohexyl, pyridinyl, piperidinyl, cyclopropyl, or tetrahydropyranyl. In some embodiments, R⁹ is a benzylic group. In some embodiments, each instance of R⁹ is independently halogen or an optionally substituted C₁₋₆ aliphatic group. In some embodiments, R⁹ is selected from those depicted in the compounds of Table 1, below.

In some embodiments, -L³-R⁸ is a substituent of Table D:

TABLE D
Exemplary-L³-R⁸ substituents

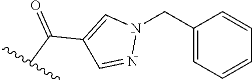 [SMILES: *C(=O)c1cnn(Cc2ccccc2)c1]

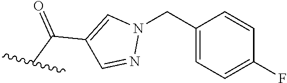 [SMILES: *C(=O)c1cnn(Cc2ccc(F)cc2)c1]

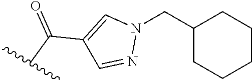 [SMILES: *C(=O)c1cnn(CC2CCCCC2)c1]

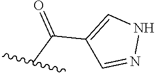 [SMILES: *C(=O)c1cn[nH]c1]

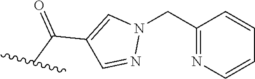 [SMILES: *C(=O)c1cnn(Cc2ccccn2)c1]

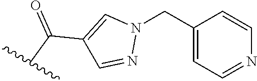 [SMILES: *C(=O)c1cnn(Cc2ccncc2)c1]

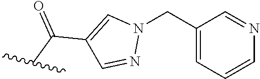 [SMILES: *C(=O)c1cnn(Cc2cccnc2)c1]

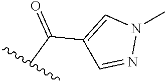 [SMILES: *C(=O)c1cnn(C)c1]

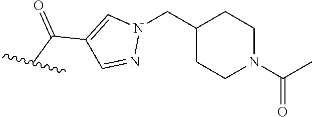 [SMILES: *C(=O)c1cnn(CC2CCN(C(C)=O)CC2)c1]

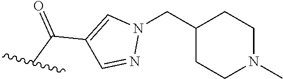 [SMILES: *C(=O)c1cnn(CC2CCN(C)CC2)c1]

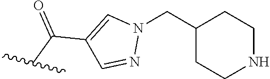 [SMILES: *C(=O)c1cnn(CC2CCNCC2)c1]

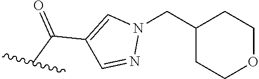 [SMILES: *C(=O)c1cnn(CC2CCOCC2)c1]

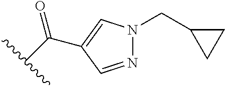 [SMILES: *C(=O)c1cnn(CC2CC2)c1]

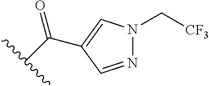 [SMILES: *C(=O)c1cnn(CC(F)(F)F)c1]

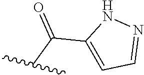 [SMILES: *C(=O)c1ccn[nH]1]

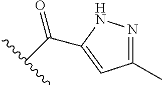 [SMILES: *C(=O)c1cc(C)n[nH]1]

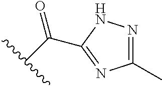 [SMILES: *C(=O)c1nc(C)n[nH]1]

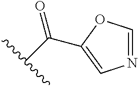 [SMILES: *C(=O)c1cnco1]

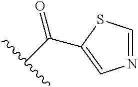 [SMILES: *C(=O)c1cncs1]

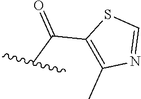 [SMILES: *C(=O)c1scnc1C]

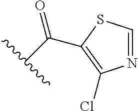 [SMILES: *C(=O)c1scnc1Cl]

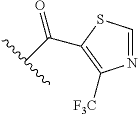 [SMILES: *C(=O)c1scnc1C(F)(F)F]

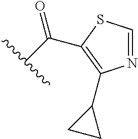 [SMILES: *C(=O)c1scnc1C1CC1]

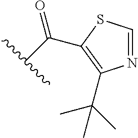 [SMILES: *C(=O)c1scnc1C(C)(C)C]

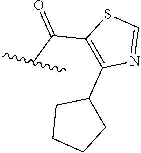 [SMILES: *C(=O)c1scnc1C1CCCC1]

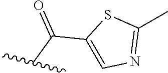 [SMILES: *C(=O)c1cnc(C)s1]

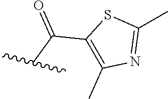 [SMILES: *C(=O)c1sc(C)nc1C]

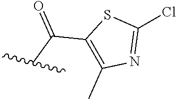 [SMILES: *C(=O)c1sc(Cl)nc1C]

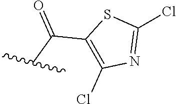 [SMILES: *C(=O)c1sc(Cl)nc1Cl]

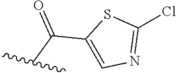 [SMILES: *C(=O)c1cnc(Cl)s1]

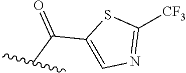 [SMILES: *C(=O)c1cnc(C(F)(F)F)s1]

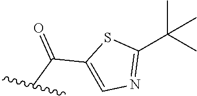 [SMILES: *C(=O)c1cnc(C(C)(C)C)s1]

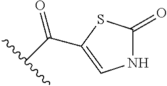 [SMILES: *C(=O)c1c[nH]c(=O)s1]

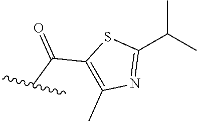 [SMILES: *C(=O)c1sc(C(C)C)nc1C]

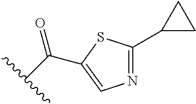 [SMILES: *C(=O)c1cnc(C2CC2)s1]

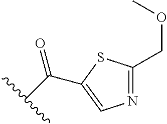 [SMILES: *C(=O)c1cnc(COC)s1]

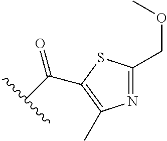 [SMILES: *C(=O)c1sc(COC)nc1C]

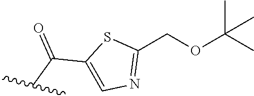 [SMILES: *C(=O)c1cnc(COC(C)(C)C)s1]

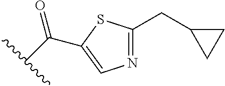 [SMILES: *C(=O)c1cnc(CC2CC2)s1]

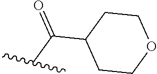 [SMILES: *C(=O)C1CCOCC1]

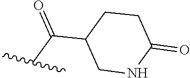 [SMILES: *C(=O)C1CCC(=O)NC1]

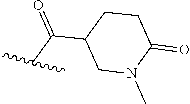 [SMILES: *C(=O)C1CCC(=O)N(C)C1]

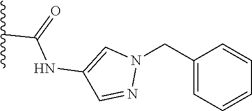 [SMILES: *C(=O)Nc1cnn(Cc2ccccc2)c1]

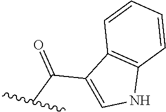 [SMILES: *C(=O)c1c[nH]c2ccccc12]

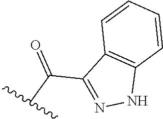 [SMILES: *C(=O)c1n[nH]c2ccccc12]

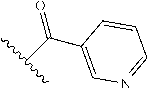 [SMILES: *C(=O)c1cccnc1]

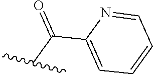 [SMILES: *C(=O)c1ccccn1]

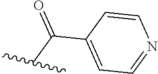 [SMILES: *C(=O)c1ccncc1]

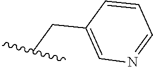 [SMILES: *Cc1cccnc1]

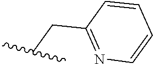 [SMILES: *Cc1ccccn1]

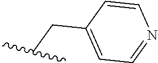 [SMILES: *Cc1ccncc1]

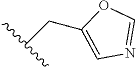 [SMILES: *Cc1cnco1]

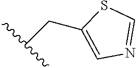 [SMILES: *Cc1cncs1]

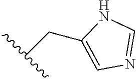 [SMILES: *Cc1cnc[nH]1]

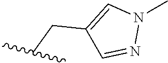 [SMILES: *Cc1cnn(C)c1]

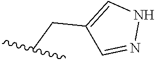 [SMILES: *Cc1cn[nH]c1]

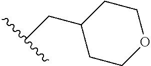 [SMILES: *CC1CCOCC1]

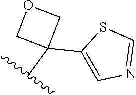 [SMILES: *C1(c2cncs2)COC1]

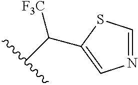 [SMILES: *C(c1cncs1)C(F)(F)F]

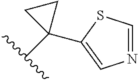 [SMILES: *C1(c2cncs2)CC1]

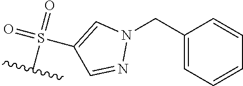 [SMILES: *S(=O)(=O)c1cnn(Cc2ccccc2)c1]

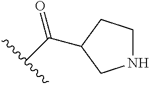 [SMILES: *C(=O)C1CCNC1]

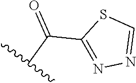 [SMILES: *C(=O)c1nncs1]

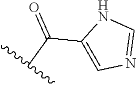 [SMILES: *C(=O)c1cnc[nH]1]

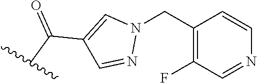 [SMILES: *C(=O)c1cnn(Cc2ccncc2F)c1]

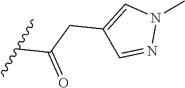 [SMILES: *C(=O)Cc1cnn(C)c1]

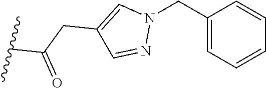 [SMILES: *C(=O)Cc1cnn(Cc2ccccc2)c1]

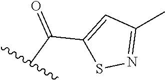 [SMILES: *C(=O)c1cc(C)ns1]

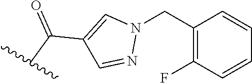 [SMILES: *C(=O)c1cnn(Cc2ccccc2F)c1]

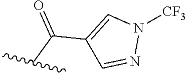 [SMILES: *C(=O)c1cnn(C(F)(F)F)c1]

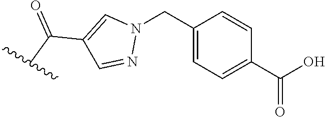 [SMILES: *C(=O)c1cnn(Cc2ccc(C(=O)O)cc2)c1]

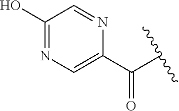 [SMILES: *C(=O)c1cnc(O)cn1]

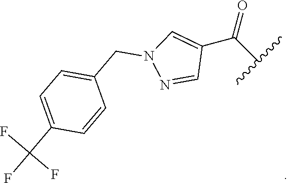 [SMILES: *C(=O)c1cnn(Cc2ccc(C(F)(F)F)cc2)c1]

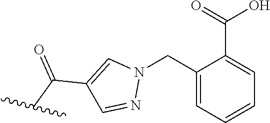 [SMILES: *C(=O)c1cnn(Cc2ccccc2C(=O)O)c1]

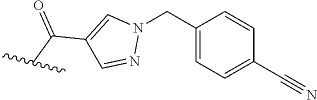 [SMILES: *C(=O)c1cnn(Cc2ccc(C#N)cc2)c1]

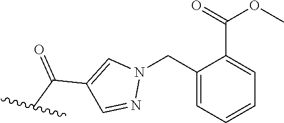 [SMILES: *C(=O)c1cnn(Cc2ccccc2C(=O)OC)c1]

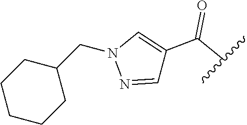 [SMILES: *C(=O)c1cnn(CC2CCCCC2)c1]

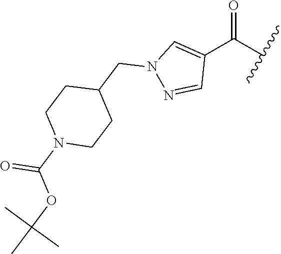 [SMILES: *C(=O)c1cnn(CC2CCN(C(=O)OC(C)(C)C)CC2)c1]

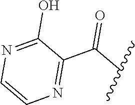 [SMILES: *C(=O)c1nccnc1O]

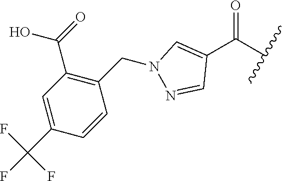 [SMILES: *C(=O)c1cnn(Cc2ccc(C(F)(F)F)cc2C(=O)O)c1]

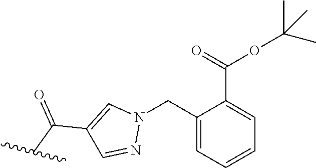 [SMILES: *C(=O)c1cnn(Cc2ccccc2C(=O)OC(C)(C)C)c1]

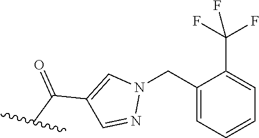 [SMILES: *C(=O)c1cnn(Cc2ccccc2C(F)(F)F)c1]

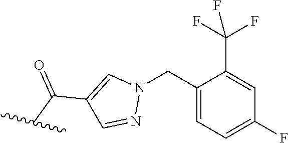 [SMILES: *C(=O)c1cnn(Cc2ccc(F)cc2C(F)(F)F)c1]

In some embodiments, -L³-R⁸ is

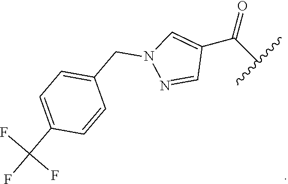 [SMILES: *C(=O)c1cnn(Cc2ccc(C(F)(F)F)cc2)c1]

In some embodiments, -L³-R⁸ is

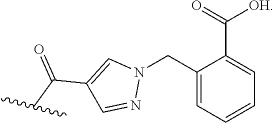 [SMILES: *C(=O)c1cnn(Cc2ccccc2C(=O)O)c1]

In some embodiments, -L¹-R⁸ is

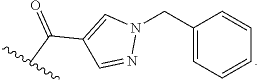 [SMILES: *C(=O)c1cnn(Cc2ccccc2)c1]

In some embodiments, -L³-R⁸ is

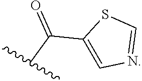 [SMILES: *C(=O)c1cncs1]

In some embodiments, the compound of Formula IA is a compound of Formula IIA:

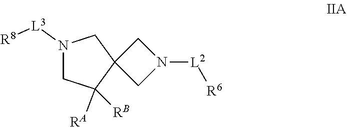

or a pharmaceutically acceptable salt thereof, wherein R^{A}, R^{B}, L², R⁶, L³ and R⁸, and their constituent groups, are each as defined and described herein. In some embodiments, R^{A}, R^{B}, L², R⁶, L³ and R⁸, and their constituent groups, are each as defined and described in Formula IA. In some embodiments, R^{A} is a substituent from Table B. In some embodiments, -L²-R⁶ is a substituent from Table C. In some embodiments, -L³-R⁸ is a substituent from Table D. In some embodiments, R^{A} is a substituent from Table B, and -L²-R⁶ is a substituent from Table C. In some embodiments, R^{A} is a substituent from Table B, and -L³-R⁸ is a substituent from Table D. In some embodiments, -L²-R⁶ is a substituent from Table C, and -L³-R⁸ is a substituent from Table D. And in some embodiments, R^{A} is a substituent from Table B, -L²-R⁶ is a substituent from Table C, and -L³-R⁸ is a substituent from Table D.

In some embodiments, the compound of Formula IA is a compound of Formula IIB:

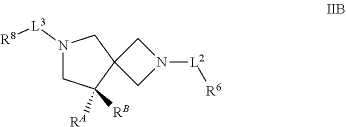

or a pharmaceutically acceptable salt thereof, wherein R^{A}, R^{B}, L², R⁶, L³ and R⁸, and their constituent groups, are each as defined and described herein. In some embodiments, R^{A}, R^{B}, L², R⁶, L³ and R⁸, and their constituent groups, are each as defined and described in Formula IA. In some embodiments, R^{A} is a substituent from Table B. In some embodiments, -L²-R⁶ is a substituent from Table C. In some embodiments, -L³-R⁸ is a substituent from Table D. In some embodiments, R^{A} is a substituent from Table B, and -L²-R⁶ is a substituent from Table C. In some embodiments, R^{A} is a substituent from Table B, and -L³-R⁸ is a substituent from Table D. In some embodiments, -L²-R⁶ is a substituent from Table C, and -L³-R⁸ is a substituent from Table D. In some embodiments, R^{A} is a substituent from Table B, -L²-R⁶ is a substituent from Table C, and -L³-R⁸ is a substituent from Table D.

In some embodiments, the compound of Formula I or IA is a compound of Formula II:

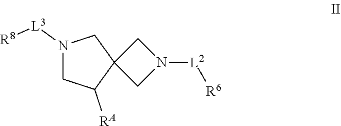

or a pharmaceutically acceptable salt thereof, wherein R^{A} L², R⁶, L³ and R⁸, and their constituent groups, are each as defined and described herein. In some embodiments, R^{A}, L², R⁶, L³ and R⁸, and their constituent groups, are each as defined and described in Formula IA. In some embodiments, R^{A} is a substituent from Table B. In some embodiments, -L²-R⁶ is a substituent from Table C. In some embodiments, -L³-R⁸ is a substituent from Table D. In some embodiments, R^{A} is a substituent from Table B, and -L²-R⁶ is a substituent from Table C. In some embodiments, R^{A} is a substituent from Table B, and -L³-R⁸ is a substituent from Table D. In some embodiments, -L²-R⁶ is a substituent from Table C, and -L³-R⁸ is a substituent from Table D. And in some embodiments, R^{A} is a substituent from Table B, -L²-R⁶ is a substituent from Table C, and -L³-R⁸ is a substituent from Table D.

In some embodiments, the compound of Formula I or IA is a compound of Formula IIIa:

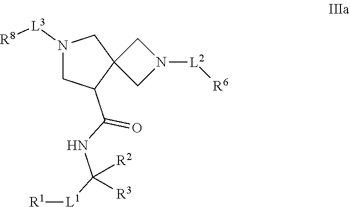

or a pharmaceutically acceptable salt thereof, wherein L¹, R¹, R², R³, L², R⁶, L³ and R⁸, and their constituent groups, are each as defined and described herein. In some embodiments, R¹ is phenyl. In some embodiments, R¹ is cyclohexyl. In some embodiments, R¹ is

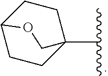 [SMILES: *C12CCC(CC1)OC2]

In some embodiments, R¹ is

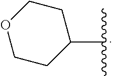 [SMILES: *C1CCOCC1]

In some embodiments, R¹ is

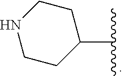 [SMILES: *C1CCNCC1]

In some embodiments, R¹ is

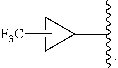

In some embodiments, R¹ is —CF₃. In some embodiments, R² is a substituent from Table A. In some embodiments, R² is —C(O)NR₂, wherein the two R groups of —C(O)NR₂, taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₄alkyl), —O(C₁₋₄alkyl), C₁₋₄haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, L² is a methylene. In some embodiments, L³ is a methylene. In some embodiments, both L² and L³ are methylene. In some embodiments, L² is a —C(O)—. In some embodiments, L³ is a —C(O)—. In some embodiments, both L² and L³ are —C(O)—. In some embodiments, R^{A} is a substituent of Table B. In some embodiments, -L²-R⁶ is

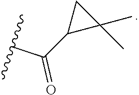 [SMILES: *C(=O)C1CC1(C)C]

In some embodiments, -L²-R⁶ is

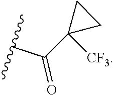 [SMILES: *C(=O)C1(C(F)(F)F)CC1]

In some embodiments, -L²-R⁶ is a substituent of Table C. In some embodiments, -L³-R⁸ is a substituent from Table D In some embodiments, -L³-R⁸ is

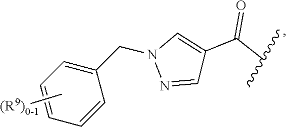

wherein R⁹ is —CF₃, —CN, —C(O)OH, —C(O)OCH₃, —C(O)OCH₂CH₃, —C(O)OC(CH₃)₃, or —C(O)OCH₂CH₂CH₃.

In some embodiments, the compound of Formula I or IA is a compound of Formula IIIb:

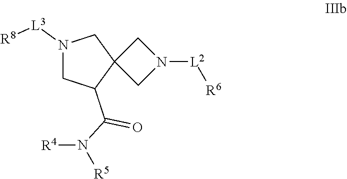

or a pharmaceutically acceptable salt thereof, wherein R⁴, R⁵, L², R⁶, L³ and R⁸, and their constituent groups, are each as defined and described herein. In some embodiments, L² is a methylene. In some embodiments, L³ is a methylene. In some embodiments, both L² and L³ are methylene. In some embodiments, L² is —C(O)—. In some embodiments, L³ is —C(O)—. In some embodiments, both L² and L³ are —C(O)—. In some embodiments, -L²-R⁶ is a substituent from Table C. In some embodiments, -L²-R⁶ is

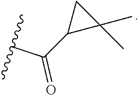 [SMILES: *C(=O)C1CC1(C)C]

In some embodiments, -L²-R⁶ is

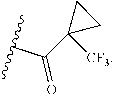 [SMILES: *C(=O)C1(C(F)(F)F)CC1]

In some embodiments, -L³-R⁸ is a substituent from Table D. In some embodiments, -L³-R⁸ is

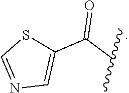 [SMILES: *C(=O)c1cncs1]

In some embodiments, R⁴ and R⁵, together, with the nitrogen to which they are attached, form an optionally substituted piperazine or an optionally substituted piperidine.

In some embodiments, the compound of Formula I or IA is a compound of Formula IVa:

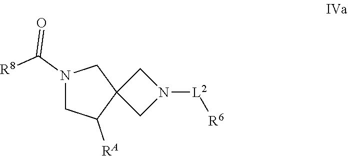

or a pharmaceutically acceptable salt thereof, wherein R^{A}, L², R⁶, and R⁸, and their constituent groups, are each as defined and described herein. In some embodiments, R^{A} is a substituent from Table B. In some embodiments, R^{A} is

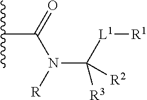

wherein R² is —C(O)NR₂, wherein the two R groups of —C(O)NR₂, taken together with the intervening nitrogen atom, form a cyclic group selected from a 4-7 membered saturated heterocyclic ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, -L²-R⁶ is a substituent from Table C. In some embodiments, -L²-R⁶ is

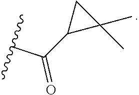 [SMILES: *C(=O)C1CC1(C)C]

In some embodiments, -L²-R⁶ is

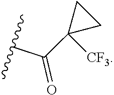 [SMILES: *C(=O)C1(C(F)(F)F)CC1]

In some embodiments, -L³-R⁸ is

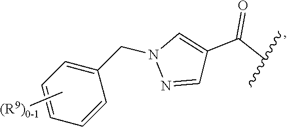

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

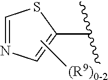

wherein R⁹ is methyl. In some embodiments, R⁸ is

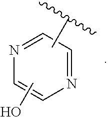

In some embodiments, the compound of Formula I is a compound of Formula IVb:

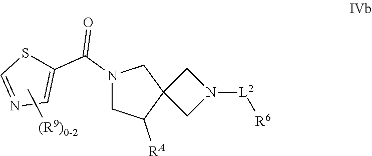

or a pharmaceutically acceptable salt thereof, wherein R^{A}, L², R⁶, and R⁹, and their constituent groups, are each as defined and described herein. In some embodiments, the thiazolyl group is not substituted with R⁹. In some embodiments, R^{A} is a substituent from Table B. In some embodiments, -L²-R⁶ is a substituent from Table C. In some embodiments, -L²-R⁶ is

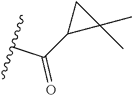 [SMILES: *C(=O)C1CC1(C)C]

In some embodiments, -L²-R⁶ is

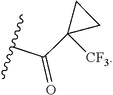 [SMILES: *C(=O)C1(C(F)(F)F)CC1]

In some embodiments, the compound of Formula I or IA is a compound of Formula IVc:

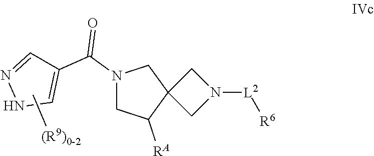

or a pharmaceutically acceptable salt thereof, wherein R^{A}, L², R⁶, and R⁹, and their constituent groups, are each as defined and described herein. In some embodiments, the pyrazolyl group is not substituted with R⁹. In some embodiments, the pyrazolyl group is substituted with one instance of R⁹, wherein R⁹ is an optionally substituted benzyl group. In some embodiments, R^{A} is a substituent from Table B. In some embodiments, -L²-R⁶ is a substituent from Table C. In some embodiments, -L²-R⁶ is

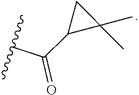 [SMILES: *C(=O)C1CC1(C)C]

In some embodiments, -L²-R⁶ is

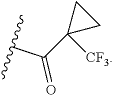 [SMILES: *C(=O)C1(C(F)(F)F)CC1]

In some embodiments, the compound of Formula I or IA is a compound of Formula Va:

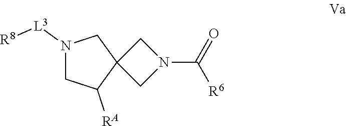

or a pharmaceutically acceptable salt thereof, wherein R^{A}, R⁶, L³ and R⁸, and their constituent groups, are each as defined and described herein. In some embodiments, R⁶ is an optionally substituted cyclopropyl group. In some embodiments, R^{A} is a substituent from Table B. In some embodiments, -L³-R⁸ is a substituent from Table D. In some embodiments, -L³-R⁸ is

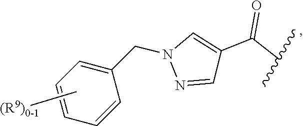

wherein R⁹ is —CF₃, —CN, —C(O)OH, —C(O)OCH₃, —C(O)OCH₂CH₃, —C(O)OC(CH₃)₃, or —C(O)OCH₂CH₂CH₃.

In some embodiments, the compound of Formula I or IA is a compound of Formula Vb:

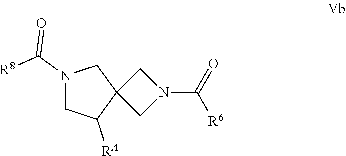

or a pharmaceutically acceptable salt thereof, wherein R^{A}, R⁶, and R⁸, and their constituent groups, are each as defined and described herein. In some embodiments, R⁶ is an optionally substituted cyclopropyl group. In some embodiments, R^{A} is a substituent from Table B. In some embodiments, R⁶ is

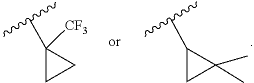

In some embodiments, R⁸ is

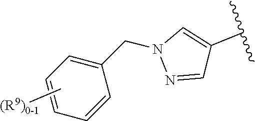

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

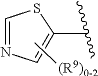

wherein R⁹ is methyl. In some embodiments, R⁸ is

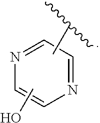

In some embodiments, the compound of Formula I or IA is a compound of Formula VIa:

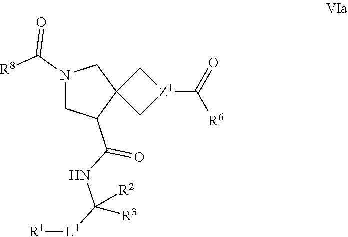

or a pharmaceutically acceptable salt thereof, wherein L¹, R¹, R², R³, R⁶, and R⁸, and their constituent groups, are each as defined and described herein. In some embodiments, L¹ is

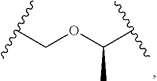 [SMILES: *CO[C@@H](*)C]

wherein the [symbol] on the left connects to R¹. In some embodiments, R⁶ is an optionally substituted cyclopropyl group. In some embodiments, L¹ is

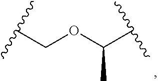 [SMILES: *CO[C@@H](*)C]

wherein the [symbol] on the left connects to R¹, wherein R¹ is

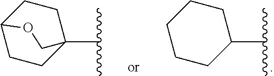 [SMILES: *C12CCC(CC1)OC2 ; *C1CCCCC1]

In some embodiments, R¹ is phenyl. In some embodiments, R¹ is cyclohexyl. In some embodiments, R¹ is

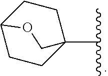 [SMILES: *C12CCC(CC1)OC2]

In some embodiments, R¹ is

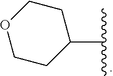 [SMILES: *C1CCOCC1]

In some embodiments, R¹ is

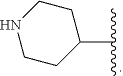 [SMILES: *C1CCNCC1]

In some embodiments, R¹ is

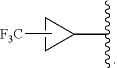

In embodiments, R¹ is —CF₃. In some embodiments, R² is a substituent from Table A. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, R² is —C(O)NR₂, wherein the two R groups of —C(O)NR₂, taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

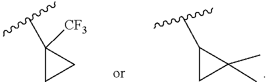

In some embodiments, R⁸ is

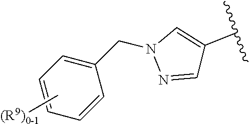

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

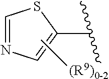

wherein R⁹ is methyl. In some embodiments, R⁸ is

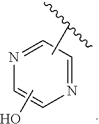

In some embodiments, the compound of Formula I or IA is a compound of Formula VIb:

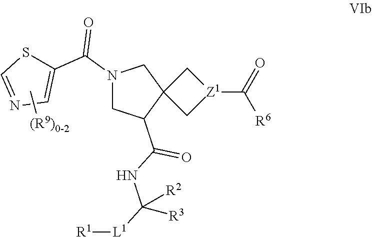

or a pharmaceutically acceptable salt thereof, wherein L¹, R¹, R², R³, R⁶, and R⁹, and their constituent groups, are each as defined and described herein. In some embodiments, R¹ is phenyl. In some embodiments, R¹ is cyclohexyl. In some embodiments, R¹ is

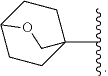 [SMILES: *C12CCC(CC1)OC2]

In some embodiments, R¹ is

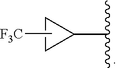

In some embodiments, R¹ is —CF₃. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

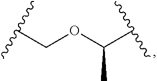 [SMILES: *CO[C@@H](*)C]

wherein the [symbol] on the left connects to R¹. In some embodiments, L¹ is

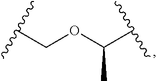 [SMILES: *CO[C@@H](*)C]

wherein the [symbol] on the left connects to R¹, wherein R¹ is or

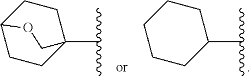 [SMILES: *C12CCC(CC1)OC2 ; *C1CCCCC1]

In some embodiments, R² is a substituent from Table A. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, the thiazolyl group is not substituted with R⁹. In some embodiments, R² is —C(O)NR₂, wherein the two R groups, —C(O)NR₂, taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

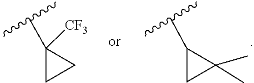

In some embodiments, the compound of Formula I or IA is a compound of Formula VIc:

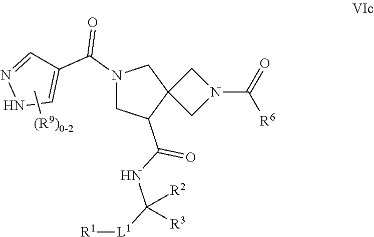

or a pharmaceutically acceptable salt thereof, wherein L¹, R¹, R², R³, R⁶, and R⁹, and their constituent groups, are each as defined and described herein. In some embodiments, R¹ is phenyl.

In some embodiments, R¹ is

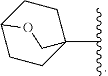 [SMILES: *C12CCC(CC1)OC2]

In some embodiments, R¹ is cyclohexyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

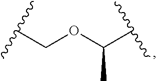 [SMILES: *CO[C@@H](*)C]

wherein the [symbol] on the left connects to R¹. In some embodiments, L¹ is

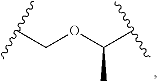 [SMILES: *CO[C@@H](*)C]

wherein the [symbol] on the left connects to R¹, wherein R¹ is

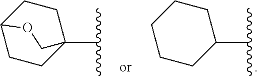 [SMILES: *C12CCC(CC1)OC2 ; *C1CCCCC1]

In some embodiments, R² is a substituent from Table A. In some embodiments, R² is —C(O)NR₂, wherein the two R groups, taken together with the intervening nitrogen atom, form an optionally substituted 4-7 membered saturated ring (having 0-3 heteroatoms, in addition to the nitrogen, independently selected from nitrogen, oxygen, and sulfur), and R³ is hydrogen. In some embodiments, the pyrazolyl group is not substituted with R⁹. In some embodiments, the pyrazolyl group is substituted with one instance of R⁹, wherein R⁹ is a benzyl group. In some embodiments, R² is —C(O)NR₂, wherein the two R groups of —C(O)NR₂, taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

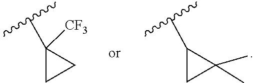

In some embodiments, the compound of Formula I or IA is a compound of Formula VId:

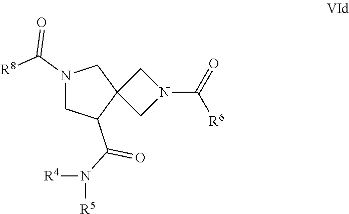

or a pharmaceutically acceptable salt thereof, wherein R⁴, R⁵, R⁶, and R⁸, and their constituent groups, are each as defined and described herein. In some embodiments, R⁶ is an optionally substituted cyclopropyl group. In some embodiments, R⁶ is

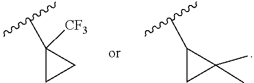

In some embodiments, R⁸ is

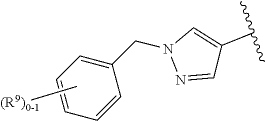

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

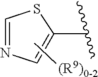

wherein R⁹ is methyl. In some embodiments, R⁸ is

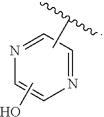

In some embodiments, R⁴ and R⁵, together, with the nitrogen to which they are attached, form an optionally substituted piperazine or an optionally substituted piperidine.

In some embodiments, the compound of Formula I or IA is a compound of Formula VIe:

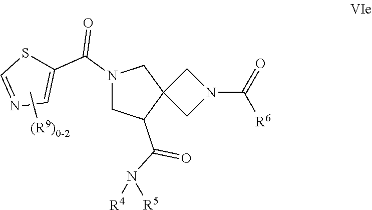

or a pharmaceutically acceptable salt thereof, wherein R⁴, R⁵, R⁶, and R⁹, and their constituent groups, are each as defined and described herein. In some embodiments, R⁶ is an optionally substituted cyclopropyl group. In some embodiments, R⁶ is

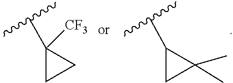

In some embodiments, R⁹ is methyl. In some embodiments, the thiazolyl group is not substituted with R⁹. In some embodiments, R⁴ and R⁵, together, with the nitrogen to which they are attached, form an optionally substituted piperazine or an optionally substituted piperidine.

In some embodiments, the compound of Formula I or IA is a compound of Formula VIf.

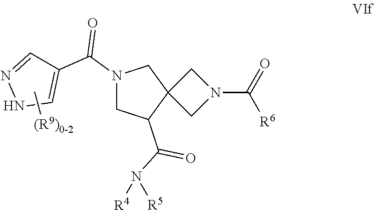

or a pharmaceutically acceptable salt thereof, wherein R⁴, R⁵, R⁶, and R⁹, and their constituent groups, are each as defined and described herein. In some embodiments, R⁶ is an optionally substituted cyclopropyl group. In some embodiments, the pyrazolyl group is not substituted with R⁹. In some embodiments, R⁶ is

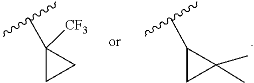

In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with CF₃, —CN, —C(O)OH, —C(O)OCH₃, —C(O)OCH₂CH₃, —C(O)OC(CH₃)₃, or —C(O)OCH₂CH₂CH₃. In some embodiments, R⁴ and R⁵, together, with the nitrogen to which they are attached, form an optionally substituted piperazine or an optionally substituted piperidine.

In some embodiments, the compound of Formula I or IA is a compound of Formula VIIa:

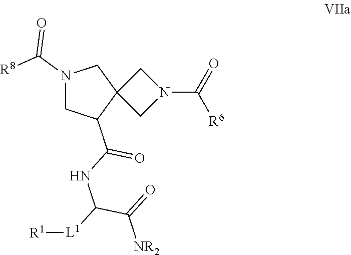

or a pharmaceutically acceptable salt thereof, wherein L¹, R¹, R², R⁶, and R⁸, and their constituent groups, are each as defined and described herein. In some embodiments, R¹ is phenyl. In some embodiments, R¹ is

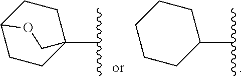

In some embodiments, R¹ is

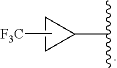

In some embodiments, R¹ is —CF₃. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

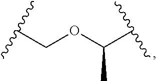 [SMILES: *CO[C@@H](*)C]

wherein the [symbol] on the left connects to R¹. In some embodiments, L¹ is

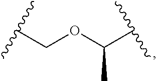 [SMILES: *CO[C@@H](*)C]

wherein the [symbol] on the left connects to R¹, wherein R¹ is

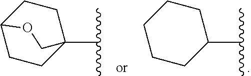 [SMILES: *C12CCC(CC1)OC2 ; *C1CCCCC1]

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁸ is

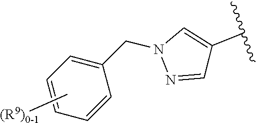

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

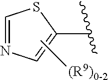

wherein R⁹ is methyl. In some embodiments, R⁸ is

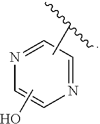

In some embodiments, R⁶ is an optionally substituted cyclopropyl group. In some embodiments, R⁶ is

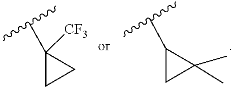

In some embodiments, the compound of Formula I or IA is a compound of Formula VIIb:

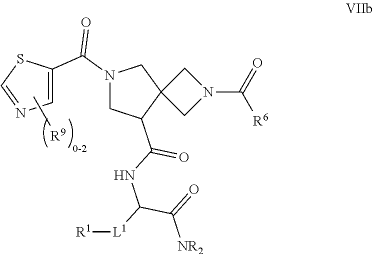

or a pharmaceutically acceptable salt thereof, wherein L¹, R¹, R², R⁶, and R⁹, and their constituent groups, are each as defined and described herein. In some embodiments, R¹ is phenyl. In some embodiments, R¹ is cyclohexyl. In some embodiments, R¹ is

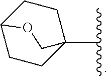 [SMILES: *C12CCC(CC1)OC2]

In some embodiments, R¹ is

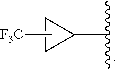

In some embodiments, R¹ is —CF₃. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

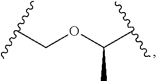 [SMILES: *CO[C@@H](*)C]

wherein the [symbol] on the left connects to R¹. In some embodiments, R⁶ is an optionally substituted cyclopropyl group. In some embodiments, L¹ is

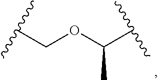 [SMILES: *CO[C@@H](*)C]

wherein the [symbol] on the left connects to R¹, wherein R¹ is

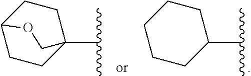 [SMILES: *C12CCC(CC1)OC2 ; *C1CCCCC1]

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

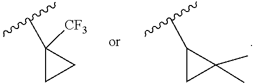

In some embodiments, the thiazolyl group is not substituted with R⁹.

In some embodiments, the compound of Formula I or IA is a compound of Formula VIIc:

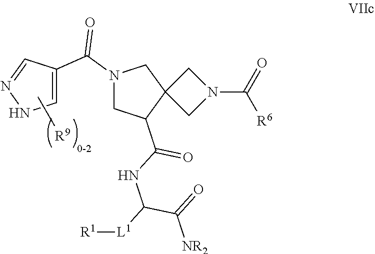

or a pharmaceutically acceptable salt thereof, wherein L¹, R¹, R², R⁶, and R⁹, and their constituent groups, are each as defined and described herein. In some embodiments, R¹ is phenyl. In some embodiments, R¹ is cyclohexyl. In some embodiments, R¹ is

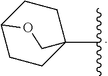 [SMILES: *C12CCC(CC1)OC2]

In some embodiments, R¹ is

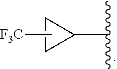

In some embodiments, R¹ is —CF₃. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

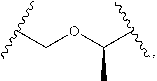 [SMILES: *CO[C@@H](*)C]

wherein the [symbol] on the left connects to R¹. In some embodiments, L¹ is

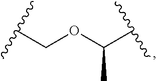 [SMILES: *CO[C@@H](*)C]

wherein the [symbol] on the left connects to R¹, wherein R¹ is

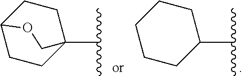

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, RP is an optionally substituted cyclopropyl group. In some embodiments, R⁶ is

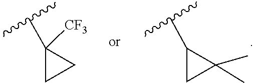

In some embodiments, the pyrazolyl group is not substituted with R⁹. In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with CF₃, —CN, —C(O)OH, —C(O)OCH₃, —C(O)OCH₂CH₃, —C(O)OC(CH₃)₃, or —C(O)OCH₂CH₂CH₃.

In some embodiments, the compound of Formula I or IA is a compound of Formula Villa:

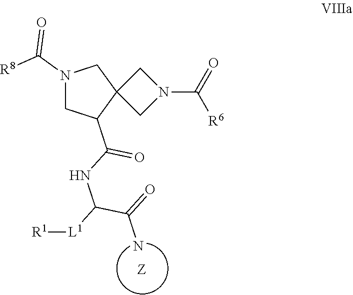

or a pharmaceutically acceptable salt thereof, wherein L¹, R¹, R⁶, and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

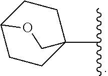 [SMILES: *C12CCC(CC1)OC2]

In some embodiments, L¹ is an optionally substituted straight or branched C₁₋₄ alkylene chain, wherein 1-2 methylene units of L¹ are independently replaced by —O—, —NR—, —C(O)O—, or —NRC(O)—In some embodiments, L¹ is

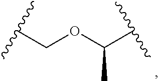 [SMILES: *CO[C@@H](*)C]

wherein the [symbol] on the left connects to R¹. In some embodiments, L¹ is

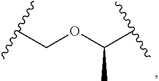 [SMILES: *CO[C@@H](*)C]

wherein the [symbol] on the left connects to R¹, wherein R¹ is

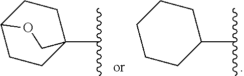

In some embodiments, R¹ is

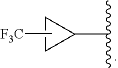

In some embodiments, R¹ is —CF₃. In some embodiments, cyclic moiety Z with the intervening nitrogen atom, forms a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R¹ is

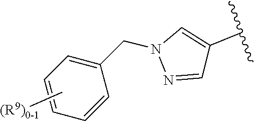

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

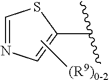

wherein R⁹ is methyl. In some embodiments, R⁹ is

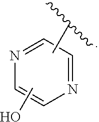

In some embodiments, R⁶ is

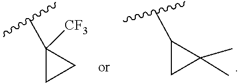

In some embodiments, R⁶ is an optionally substituted cyclopropyl group. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula VIIIb:

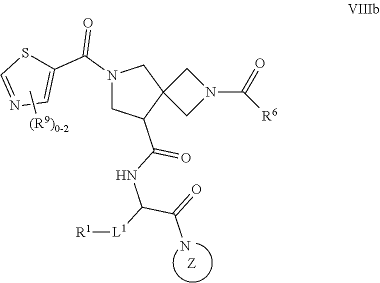

or a pharmaceutically acceptable salt thereof, wherein L¹, R¹, and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is

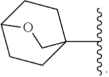 [SMILES: *C12CCC(CC1)OC2]

In some embodiments, R¹ is

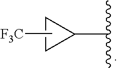

In some embodiments, R¹ is —CF₃. In some embodiments, R¹ is cyclohexyl. In some embodiments, L¹ is an optionally substituted straight or branched C₁₋₄ alkylene chain, wherein 1-2 methylene units of L¹ are independently replaced by —O—, —NR—, —C(O)O—, or —NRC(O)—. In some embodiments, L¹ is

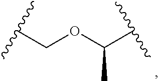 [SMILES: *CO[C@@H](*)C]

wherein the [symbol] on the left connects to R¹. In some embodiments, L¹ is

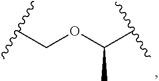 [SMILES: *CO[C@@H](*)C]

wherein the [symbol] on the left connects to R¹, wherein R¹ is

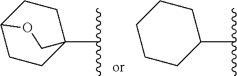

In some embodiments, the cyclic moiety Z taken together with the intervening nitrogen atom, forms a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

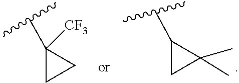

In some embodiments, the thiazolyl group is not substituted with R⁹. In some embodiments, the thiazolyl group is substituted with 0-1 R⁹ instances which are methyl. In some embodiments, Z, or in any of the aforementioned embodiments of this paragraph, is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula VIIIc:

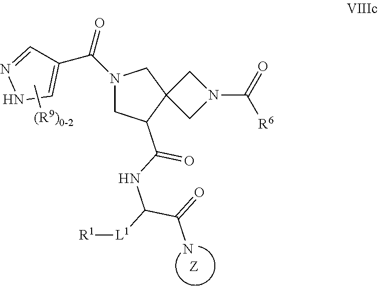

or a pharmaceutically acceptable salt thereof, wherein L¹, R¹, R⁶, and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

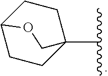 [SMILES: *C12CCC(CC1)OC2]

In some embodiments, L¹ is an optionally substituted straight or branched C₁₋₄ alkylene chain, wherein 1-2 methylene units of L¹ are independently replaced by —O—, —NR—, —C(O)O—, or —NRC(O)—.
In some embodiments, L¹ is

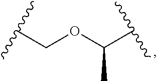 [SMILES: *CO[C@@H](*)C]

wherein the [symbol] on the left connects to R¹. In some embodiments, L¹ is

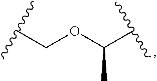 [SMILES: *CO[C@@H](*)C]

wherein the [symbol] on the left connects to R¹, wherein R¹ is

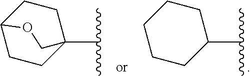 [SMILES: *C12CCC(CC1)OC2 ; *C1CCCCC1]

In some embodiments, R⁸ is

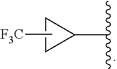

In some embodiments, R⁸ is —CF₃. In some embodiments, the cyclic moiety Z taken together with the intervening nitrogen atom, forms a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

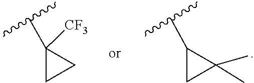

In some embodiments, the pyrazolyl group is not substituted with R⁹. In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with CF₃, —CN, —C(O)OH, —C(O)OCH₃, —C(O)OCH₂CH₃, or —C(O)OCH₂CH₂CH₃. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula IXa:

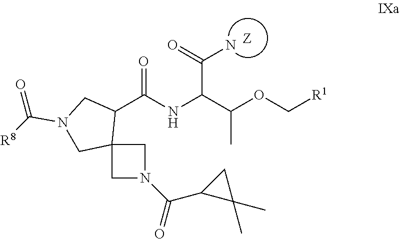

or a pharmaceutically acceptable salt thereof, wherein R¹ and R⁸, and their constituent groups, are each as defined and described herein, and cyclic moiety Z is an optionally substituted cyclic group formed from two R groups, as defined and described herein. In some embodiments, R¹ is phenyl.

In some embodiments, R¹ is

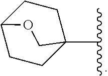 [SMILES: *C12CCC(CC1)OC2]

In some embodiments, R¹ is

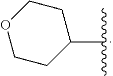 [SMILES: *C1CCOCC1]

In some embodiments, R¹ is

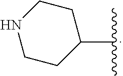 [SMILES: *C1CCNCC1]

In some embodiments, R¹ is cyclohexyl. In some embodiments, R¹ is

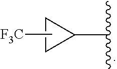

In some embodiments, R¹ is —CF₃. In some embodiments, R⁸ is

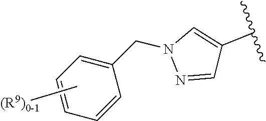

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

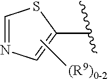

wherein R⁹ is methyl. In some embodiments, R⁸ is

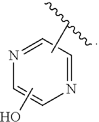

In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula IXa*:

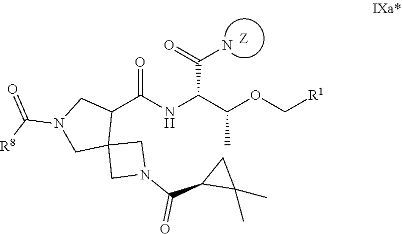

or a pharmaceutically acceptable salt thereof, wherein R¹ and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

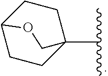 [SMILES: *C12CCC(CC1)OC2]

In some embodiments, R¹ is

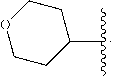 [SMILES: *C1CCOCC1]

In some embodiments, R¹ is

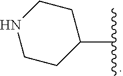 [SMILES: *C1CCNCC1]

In some embodiments, R¹ is

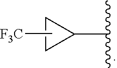

In some embodiments, R¹ is —CF₃. In some embodiments, R⁸ is

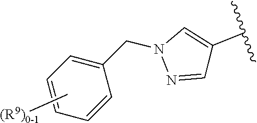

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

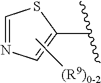

wherein R⁹ is methyl. In some embodiments, R⁸ is

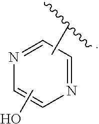

In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula IXa**:

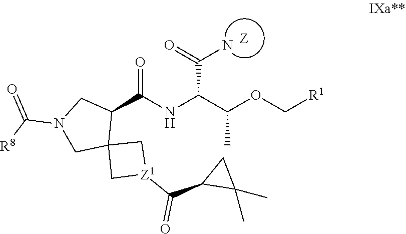

or a pharmaceutically acceptable salt thereof, wherein R¹ and R⁸, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

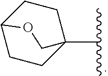 [SMILES: *C12CCC(CC1)OC2]

In some embodiments, R¹ is

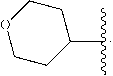 [SMILES: *C1CCOCC1]

In some embodiments, R¹ is

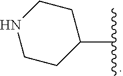 [SMILES: *C1CCNCC1]

In some embodiments, R¹ is

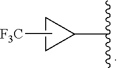

In some embodiments, R¹ is —CF₃. In some embodiments, R⁸ is

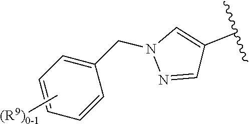

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

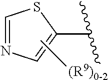

wherein R⁹ is methyl. In some embodiments, R⁸ is

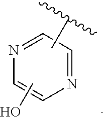

In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula IXb:

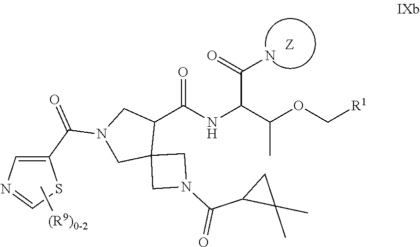

or a pharmaceutically acceptable salt thereof, wherein R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

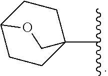 [SMILES: *C12CCC(CC1)OC2]

In some embodiments, R¹ is

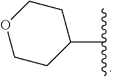 [SMILES: *C1CCOCC1]

In some embodiments, R¹ is

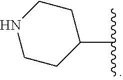 [SMILES: *C1CCNCC1]

In some embodiments, R¹ is

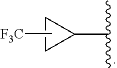

In some embodiments, R¹ is —CF₃. In some embodiments, the thiazolyl group is not substituted with R⁹. In some embodiments, the thiazolyl group is substituted with one instance of R⁹, wherein R⁹ is a benzyl group. In some embodiments, the thiazolyl group is substituted with one or two instances of R⁹, which are methyl groups. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ is wherein the R⁹ is an optionally substituted benzyl or an optionally substituted cyclic group selected from phenyl and 5-6 membered heteroaryl.

In some embodiments, the compound of Formula I or IA is a compound of Formula IXb*:

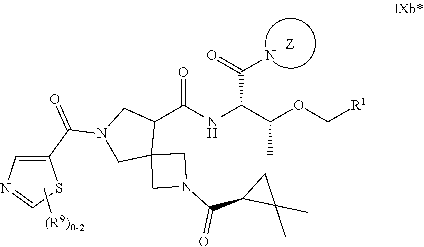

or a pharmaceutically acceptable salt thereof, wherein R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

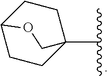 [SMILES: *C12CCC(CC1)OC2]

In some embodiments, R¹ is

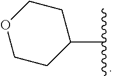 [SMILES: *C1CCOCC1]

In some embodiments, R¹ is

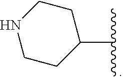 [SMILES: *C1CCNCC1]

In some embodiments, R¹ is

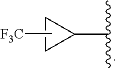

In some embodiments, R¹ is —CF₃. In some embodiments, the thiazolyl group is not substituted with R In some embodiments, the thiazolyl group is substituted with one instance of R⁹, wherein R⁹ is a benzyl group. In some embodiments, the thiazolyl group is substituted with one or two instances of R⁹, which are methyl groups. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula IXb**:

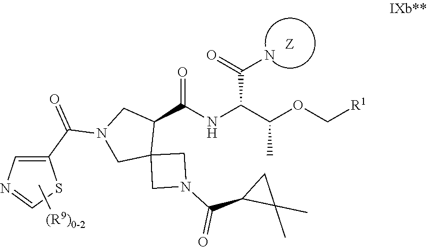

or a pharmaceutically acceptable salt thereof, wherein R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

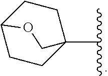 [SMILES: *C12CCC(CC1)OC2]

In some embodiments, R¹ is

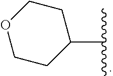 [SMILES: *C1CCOCC1]

In some embodiments, R¹ is

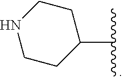 [SMILES: *C1CCNCC1]

In some embodiments, R¹ is

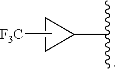

In some embodiments, R¹ is —CF₃. In some embodiments, the thiazolyl group is not substituted with R⁹. In some embodiments, the thiazolyl group is substituted with one instance of R⁹, wherein R⁹ is a benzyl group. In some embodiments, the thiazolyl group is substituted with one or two instances of R⁹, which are methyl groups. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula IXc.

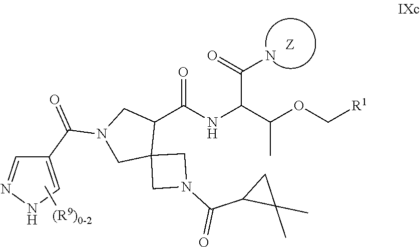

or a pharmaceutically acceptable salt thereof, wherein R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

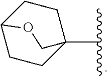 [SMILES: *C12CCC(CC1)OC2]

In some embodiments, R¹ is

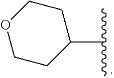 [SMILES: *C1CCOCC1]

In some embodiments, R¹ is

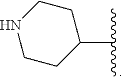 [SMILES: *C1CCNCC1]

In some embodiments, R¹ is

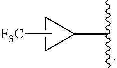

In some embodiments, R¹ is —CF₃. In some embodiments, the pyrazolyl group is not substituted with R⁹. In some embodiments, the pyrazolyl group is substituted with one instance of R⁹, wherein R⁹ is a benzyl group. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula IXc*:

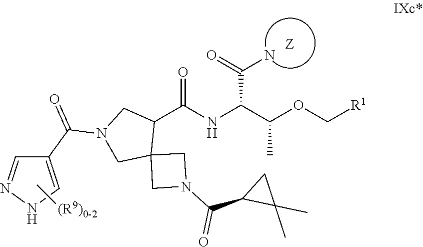

or a pharmaceutically acceptable salt thereof, wherein R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is cyclohexyl. In some embodiments, R¹ is

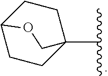 [SMILES: *C12CCC(CC1)OC2]

In some embodiments, R¹ is

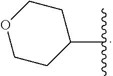 [SMILES: *C1CCOCC1]

In some embodiments, R¹ is

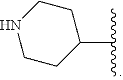 [SMILES: *C1CCNCC1]

In some embodiments, R¹ is

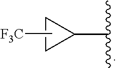

In some embodiments, R¹ is —CF₃. In some embodiments, the pyrazolyl group is not substituted with R⁹. In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with —CF₃, —CN, —C(O)OH, —C(O)OCH₃, —C(O)OCH₂CH₃, or —C(O)OCH₂CH₂CH₃. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula IXc**.

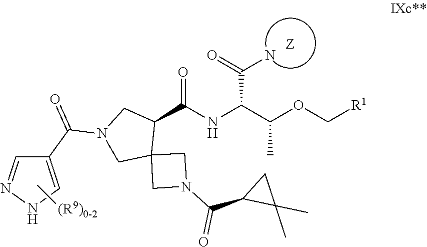

or a pharmaceutically acceptable salt thereof, wherein R¹ and R⁹, and their constituent groups, are each as defined and described herein, and cyclic moiety Z is an optionally substituted cyclic group formed from two R groups, as defined and described herein. In some embodiments, R¹ is phenyl. In some embodiments, R¹ is

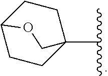 [SMILES: *C12CCC(CC1)OC2]

In some embodiments, R¹ is

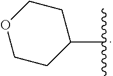 [SMILES: *C1CCOCC1]

In some embodiments, R¹ is

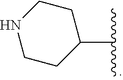 [SMILES: *C1CCNCC1]

In some embodiments, R¹ is

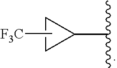

In some embodiments, R¹ is —CF₃. In some embodiments, R¹ is cyclohexyl. In some embodiments, the pyrazolyl group is not substituted with R⁹. In some embodiments, the pyrazolyl group is substituted with one instance of R⁹, wherein R⁹ is a benzyl group. In some embodiments, the pyrazolyl group is substituted with one instance of R⁹, wherein R⁹ is a benzyl group substituted with —CF₃, —CN, —C(O)OH, —C(O)OCH₃, —C(O)OCH₂CH₃, or —C(O)OCH₂CH₂CH₃. In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula I or IA is a compound of Formula Xa:

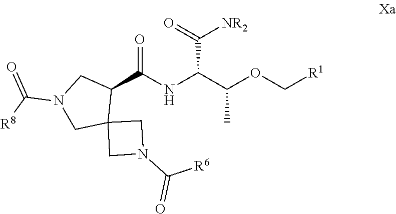

or a pharmaceutically acceptable salt thereof, wherein R¹, R⁶, and R⁸, and their constituent groups, are each as defined and described herein. In some embodiments, R¹ is phenyl. In some embodiments, R¹ is cyclohexyl. In some embodiments, R¹ is

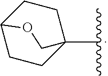 [SMILES: *C12CCC(CC1)OC2]

In some embodiments, R¹ is

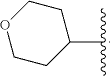 [SMILES: *C1CCOCC1]

In some embodiments, R¹ is

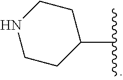 [SMILES: *C1CCNCC1]

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, an optionally substituted C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

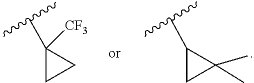

In some embodiments, R⁸ is

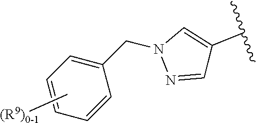

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

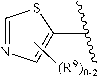

wherein R⁹ is methyl. In some embodiments, R⁸ is

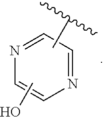

In some embodiments, the compound of Formula I or IA is a compound of Formula Xb:

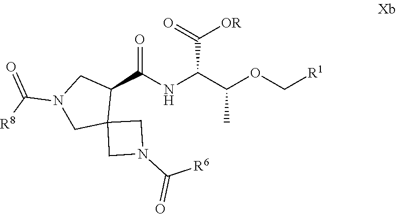

or a pharmaceutically acceptable salt thereof, wherein R¹, R⁶, and R⁸, and their constituent groups, are each as defined and described herein. In some embodiments, R¹ is phenyl. In some embodiments, R¹ is cyclohexyl. In some embodiments, R¹ is

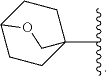 [SMILES: *C12CCC(CC1)OC2]

In some embodiments, R¹ is

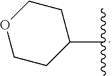 [SMILES: *C1CCOCC1]

In some embodiments, R¹ is

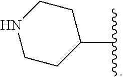 [SMILES: *C1CCNCC1]

In some embodiments, R⁶ is an optionally substituted cyclopropyl group. In some embodiments, R⁶ is

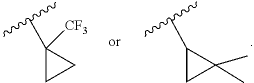

In some embodiments, R⁸ is

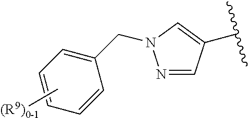

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

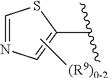

wherein R⁹ is methyl. In some embodiments, R⁸ is

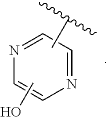

In some embodiments, the compound of Formula I or IA is a compound of Formula Xc:

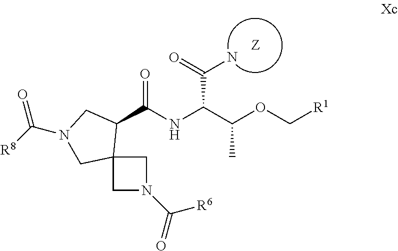

or a pharmaceutically acceptable salt thereof, wherein Z, R¹, R⁶, and R⁸, and their constituent groups, are each as defined and described herein. In some embodiments, R¹ is phenyl. In some embodiments, R¹ is cyclohexyl. In some embodiments, R¹ is

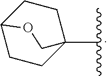 [SMILES: *C12CCC(CC1)OC2]

In some embodiments, R¹ is

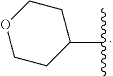 [SMILES: *C1CCOCC1]

In some embodiments, R¹ is

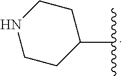 [SMILES: *C1CCNCC1]

In some embodiments, R⁶ is an optionally substituted cyclopropyl group. In some embodiments, R⁶ is

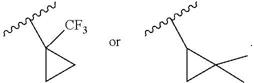

In some embodiments, R⁸ is

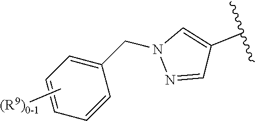

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

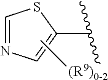

wherein R⁹ is methyl. In some embodiments, R⁸ is

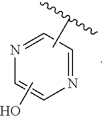

In some embodiments, or in any of the aforementioned embodiments of this paragraph, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula IA is a compound of Formula XIa:

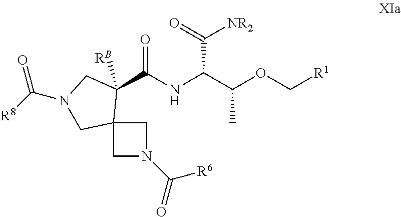

or a pharmaceutically acceptable salt thereof, wherein R^{B}, R¹, R⁶, and R⁸, and their constituent groups, are each as defined and described herein. In some embodiments, R¹ is phenyl. In some embodiments, R¹ is cyclohexyl. In some embodiments, R¹ is

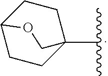 [SMILES: *C12CCC(CC1)OC2]

In some embodiments, R¹ is

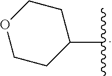 [SMILES: *C1CCOCC1]

In some embodiments, R¹ is

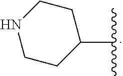 [SMILES: *C1CCNCC1]

In some embodiments, the two R groups taken together with the intervening nitrogen atom, form a cyclic group selected from an optionally substituted 4-7 membered saturated ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹, wherein R⁹ is selected from —CN, —C(O)O(C₁₋₃alkyl), —O(C₁₋₃alkyl), C₁₋₃haloalkyl, halo, C₁₋₆ aliphatic group, an optionally substituted cyclic group selected from phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and R³ is hydrogen. In some embodiments, R⁶ is an optionally substituted cyclopropyl group. In some embodiments, R⁶ is

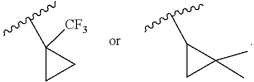

In some embodiments, R⁸ is

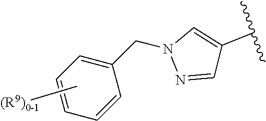

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

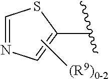

wherein R⁹ is methyl. In some embodiments, R⁸ is

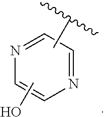

In some embodiments, the compound of Formula IA is a compound of Formula XIb:

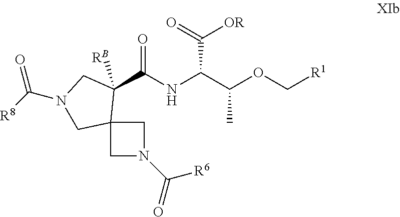

or a pharmaceutically acceptable salt thereof, wherein R^{B}, R¹, R⁶, and R⁸, and their constituent groups, are each as defined and described herein. In some embodiments, R¹ is phenyl. In some embodiments, R¹ is cyclohexyl. In some embodiments, R¹ is

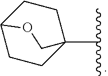 [SMILES: *C12CCC(CC1)OC2]

In some embodiments, R¹ is

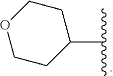 [SMILES: *C1CCOCC1]

In some embodiments, R¹ is

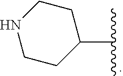 [SMILES: *C1CCNCC1]

In some embodiments, R⁶ is an optionally substituted cyclopropyl group. In some embodiments, R⁶ is

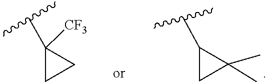

In some embodiments, R⁸ is

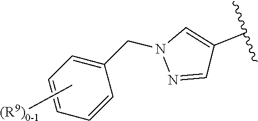

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

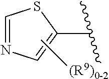

wherein R⁹ is methyl. In some embodiments, R⁸ is

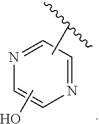

In some embodiments, the compound of Formula IA is a compound of Formula XIc:

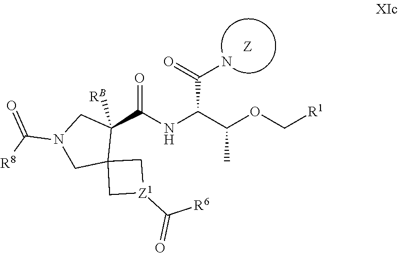

or a pharmaceutically acceptable salt thereof, wherein Z, R^{B}, R¹, R⁶, and R⁸, and their constituent groups, are each as defined and described herein. In some embodiments, R¹ is phenyl. In some embodiments, R¹ is cyclohexyl. In some embodiments, R¹ is

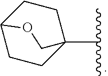 [SMILES: *C12CCC(CC1)OC2]

In some embodiments, R¹ is

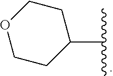 [SMILES: *C1CCOCC1]

In some embodiments, R¹ is

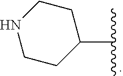 [SMILES: *C1CCNCC1]

In some embodiments, R⁶ is an optionally substituted cyclopropyl group. In some embodiments, R⁶ is

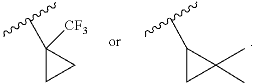

In some embodiments, R⁸ is

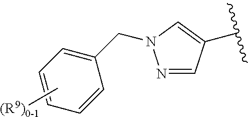

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

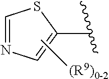

wherein R⁹ is methyl. In some embodiments, R⁸ is

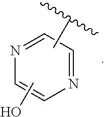

In some embodiments, Z is an optionally substituted cyclic group selected from piperidinyl, morpholinyl, piperazinyl, azetindinyl, pyrrolidinyl, azaspiro[3.3]heptanyl, and diazaspiro[3.3]heptanyl. In some embodiments, or in any of the aforementioned embodiments of this paragraph, Z is piperidinyl or piperazinyl substituted with one instance of R⁹ wherein the R⁹ is an optionally substituted benzyl or an optionally substituted cyclic group selected from phenyl and 5-6 membered heteroaryl.

In some embodiments, the compound of Formula IA is a compound of Formula XIIa or XIIb:

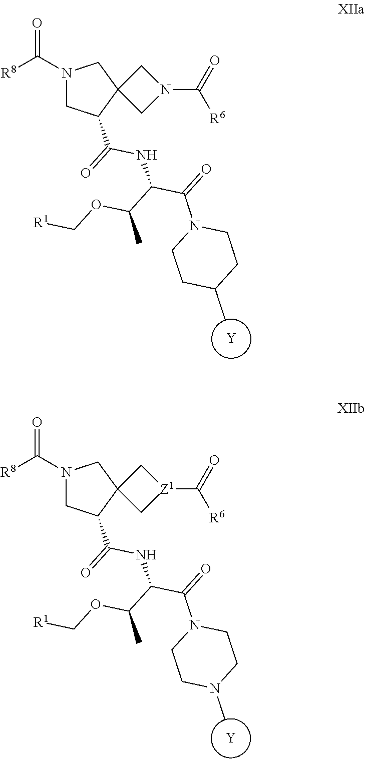

or a pharmaceutically acceptable salt thereof, wherein R¹, R⁶, and R⁸, and their constituent groups, are each as defined and described herein, and wherein

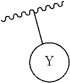

is —CH₂—Cy or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur). In some embodiments, R¹ is phenyl. In some embodiments, R¹ is cyclohexyl. In some embodiments, R¹ is

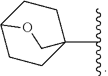 [SMILES: *C12CCC(CC1)OC2]

In some embodiments, R¹ is

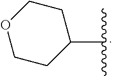 [SMILES: *C1CCOCC1]

In some embodiments, R¹ is

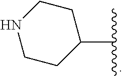 [SMILES: *C1CCNCC1]

In some embodiments, R⁶ is an optionally substituted cyclopropyl group. In some embodiments, R⁶ is

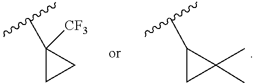

In some embodiments, R⁸ is

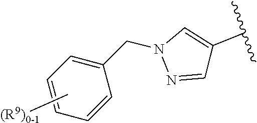

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

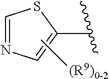

wherein R⁹ is methyl. In some embodiments, R⁸ is

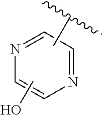

In some embodiments, the compound of Formula IA is a compound of Formula XIIIa, XIIIb, XIIIc, or XIIId:

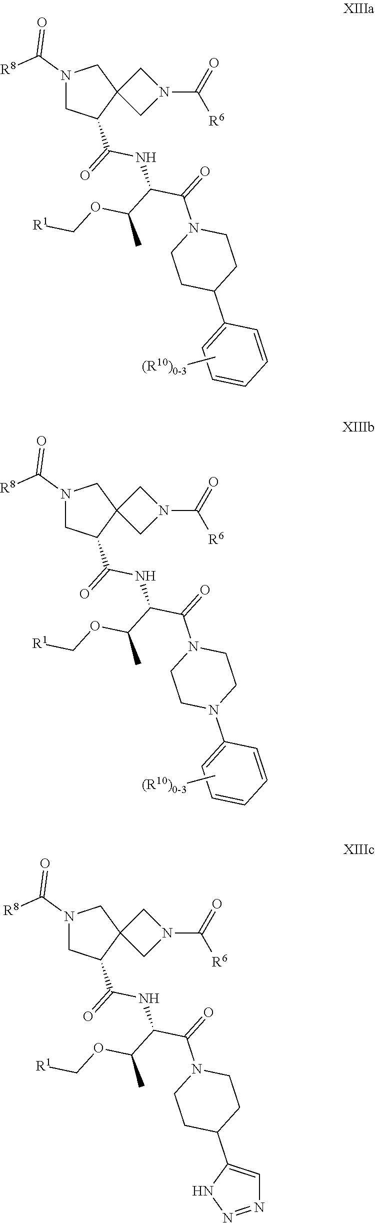

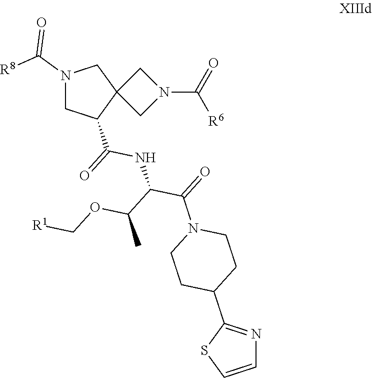

or a pharmaceutically acceptable salt thereof, wherein R¹, R⁶, and R⁸, and their constituent groups, are each as defined and described herein, and wherein R¹⁰ has from 0 to 3 instances each independently selected from halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, and Cy. In some embodiments, R¹⁰ has from 0 to 3 instances each independently selected from —F, —CF₃, —C(O)OH, —C(O)OCH₃, —C(O)OCH₂CH₃, —C(O)OCH₂CH₂CH₃, —C(O)OC(CH₃)₃, —OCH₂C(O)OH, —OCH₂C(O)OCH₃, —CH₃, —OC(CH₃)₂C(O)OH, —OC(CH₃)₂C(O)OCH₃. In some embodiments, R¹ is cyclohexyl. In some embodiments, R¹ is

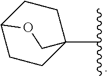 [SMILES: *C12CCC(CC1)OC2]

In some embodiments, R¹ is

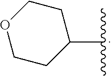 [SMILES: *C1CCOCC1]

In some embodiments, R¹ is

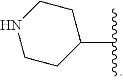 [SMILES: *C1CCNCC1]

In some embodiments, R⁶ is an optionally substituted cyclopropyl group. In some embodiments, R⁶ is

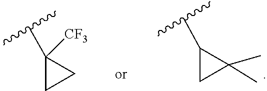

In some embodiments, R⁸ is

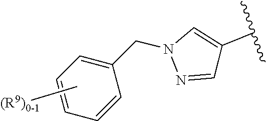

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

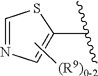

wherein R⁹ is methyl. In some embodiments, R⁸ is

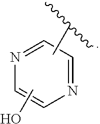

In some embodiments, the compound of Formula IA is a compound of Formula XIV:

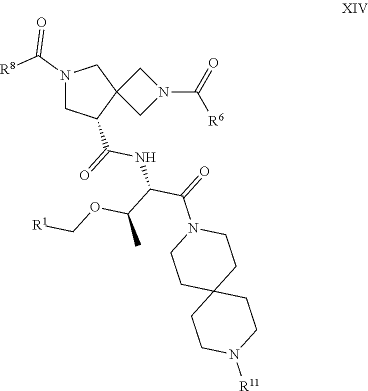

or a pharmaceutically acceptable salt thereof, wherein R¹, R⁶, and R⁸, and their constituent groups, are each as defined and described herein, and wherein R¹¹ is hydrogen, —S(O)₂R, —S(O)₂NR₂, —S(O)R, —S(O)NR₂, —C(O)R, —C(O)OR, —C(O)NR₂, —C(O)N(R)OR, an optionally substituted C₁₋₆ aliphatic group, an optionally substituted C₁₋₆ aliphatic-Cy group, or Cy. In some embodiments, R¹¹ is hydrogen, —C(O)OR, or an optionally substituted C₁₋₆ aliphatic group. In some embodiments, R¹ is cyclohexyl. In some embodiments, R¹ is

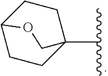 [SMILES: *C12CCC(CC1)OC2]

In some embodiments, R¹ is

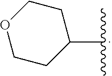 [SMILES: *C1CCOCC1]

In some embodiments, R⁸ is

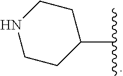 [SMILES: *C1CCNCC1]

In some embodiments, R⁶ is an optionally substituted cyclopropyl group. In some embodiments, R⁶ is

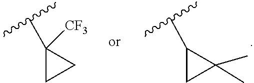

In some embodiments, R⁸ is

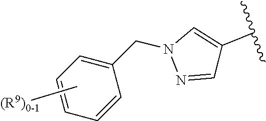

wherein R⁹ is —CF₃, —CN, —C(O)OH, —C(O)OCH₃, —C(O)OCH₂CH₃, —C(O)OC(CH₃)₃, or —C(O)OCH₂CH₂CH₃. In some embodiments, R⁸ is

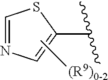

wherein R⁹ is methyl. In some embodiments, R⁸ is

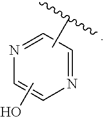

In some embodiments, at least one hydrogen atom of the compound is a deuterium atom. In some embodiments, at least one C₁-C₆ aliphatic group of the compound is substituted with at least one deuterium atom. In some embodiments, at least one C₁-C₆ alkyl group of the compound is substituted with at least one deuterium atom. In some embodiments, at least one C₁-C₆ alkylene group of the compound is substituted with at least one deuterium atom. In some embodiments, at least one bivalent C₁₋₆ hydrocarbon chain group of the compound is substituted with at least one deuterium atom. In some embodiments, R^{B} is —CD₃. In some embodiments, R² is substituted with one or more deuterium atoms. In some embodiments, R¹ is substituted with one or more deuterium atoms.

Exemplary compounds of the present disclosure are set forth in Table 1, below.

TABLE 1
Exemplary Compounds

| Cmpd # | Structure |
|---|---|
| I-1 | 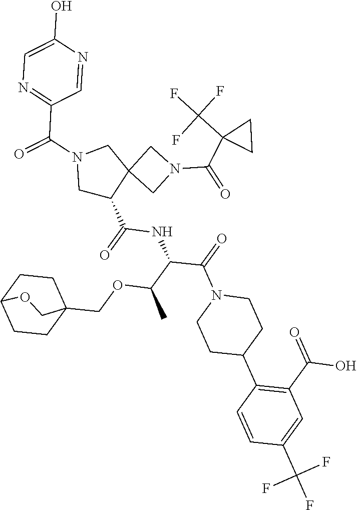 [SMILES: C[C@@H](OCC12CCC(CC1)OC2)[C@H](NC(=O)[C@@H]1CN(C(=O)c2cnc(O)cn2)CC12CN(C(=O)C1(C(F)(F)F)CC1)C2)C(=O)N1CCC(c2ccc(C(F)(F)F)cc2C(=O)O)CC1] |
| I-2 | 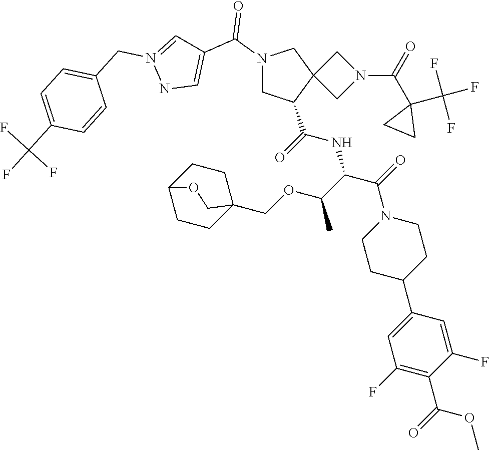 |
| I-3 | 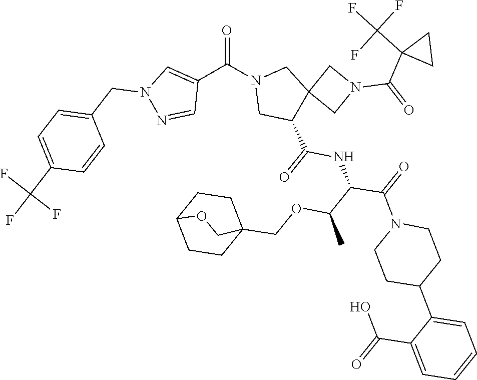 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccccc2C(=O)O)CC1] |
| I-4 | 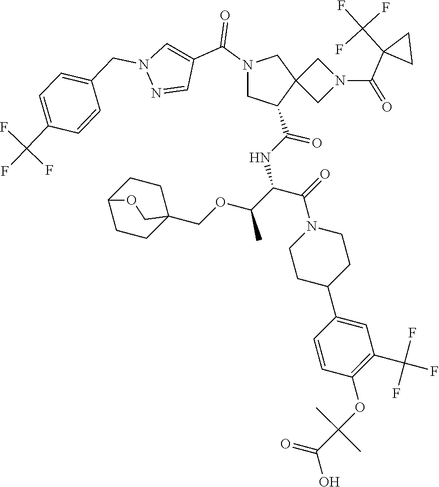 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccc(OC(C)(C)C(=O)O)c(C(F)(F)F)c2)CC1] |
| I-5 | 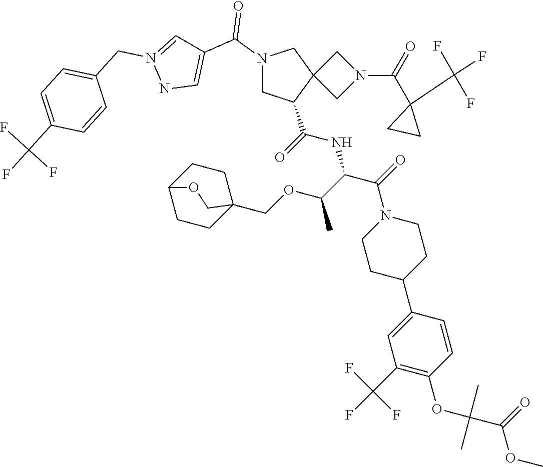 |
| I-6 | 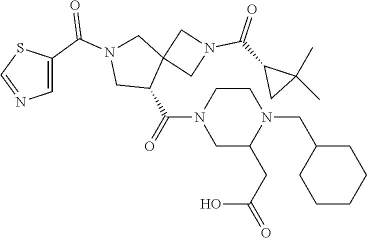 [SMILES: CC1(C)C[C@@H]1C(=O)N1CC2(CN(C(=O)c3cncs3)C[C@H]2C(=O)N2CCN(CC3CCCCC3)C(CC(=O)O)C2)C1] |
| I-7 | 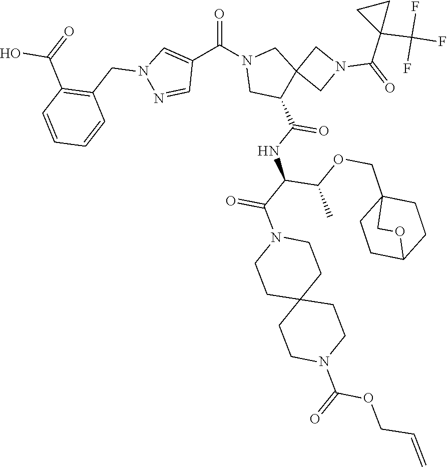 [SMILES: C=CCOC(=O)N1CCC2(CC1)CCN(C(=O)[C@@H](NC(=O)[C@@H]1CN(C(=O)c3cnn(Cc4ccccc4C(=O)O)c3)CC13CN(C(=O)C1(C(F)(F)F)CC1)C3)[C@@H](C)OCC13CCC(CC1)OC3)CC2] |
| I-8 | 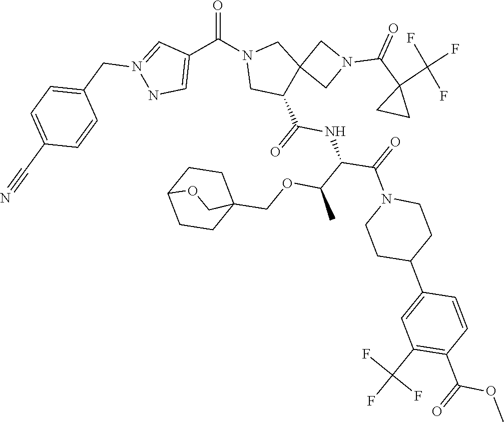 |
| I-9 | 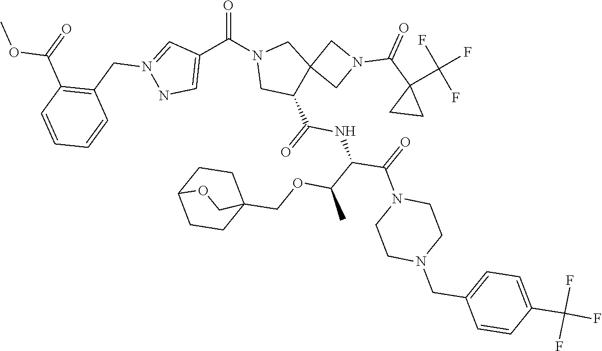 |
| I-10 | 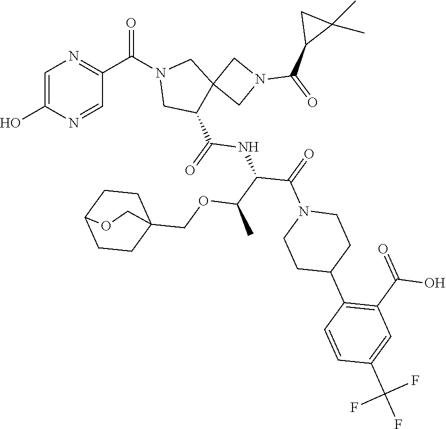 [SMILES: C[C@@H](OCC12CCC(CC1)OC2)[C@H](NC(=O)[C@@H]1CN(C(=O)c2cnc(O)cn2)CC12CN(C(=O)[C@H]1CC1(C)C)C2)C(=O)N1CCC(c2ccc(C(F)(F)F)cc2C(=O)O)CC1] |
| I-11 | 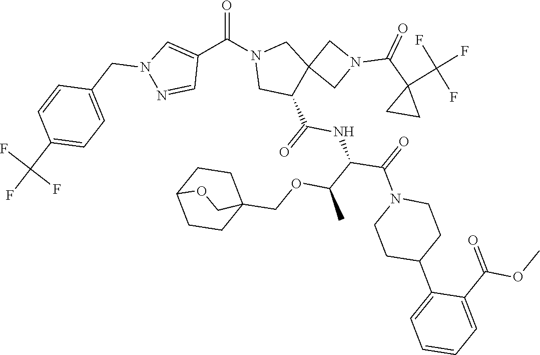 [SMILES: COC(=O)c1ccccc1C1CCN(C(=O)[C@@H](NC(=O)[C@@H]2CN(C(=O)c3cnn(Cc4ccc(C(F)(F)F)cc4)c3)CC23CN(C(=O)C2(C(F)(F)F)CC2)C3)[C@@H](C)OCC23CCC(CC2)OC3)CC1] |
| I-12 | 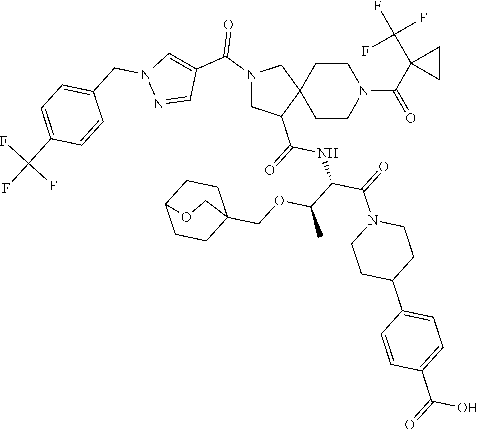 [SMILES: C[C@@H](OCC12CCC(CC1)OC2)[C@H](NC(=O)C1CN(C(=O)c2cnn(Cc3ccc(C(F)(F)F)cc3)c2)CC12CCN(C(=O)C1(C(F)(F)F)CC1)CC2)C(=O)N1CCC(c2ccc(C(=O)O)cc2)CC1] |
| I-13 | 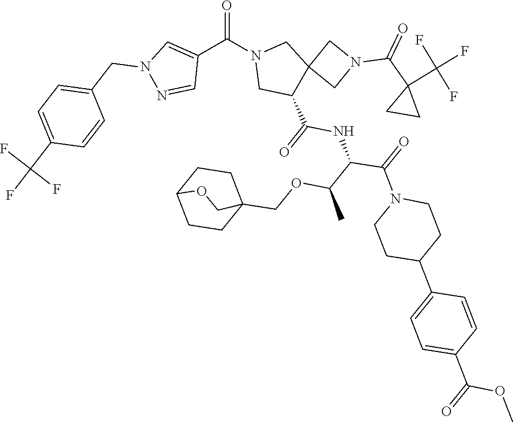 [SMILES: COC(=O)c1ccc(C2CCN(C(=O)[C@@H](NC(=O)[C@@H]3CN(C(=O)c4cnn(Cc5ccc(C(F)(F)F)cc5)c4)CC34CN(C(=O)C3(C(F)(F)F)CC3)C4)[C@@H](C)OCC34CCC(CC3)OC4)CC2)cc1] |
| I-14 | 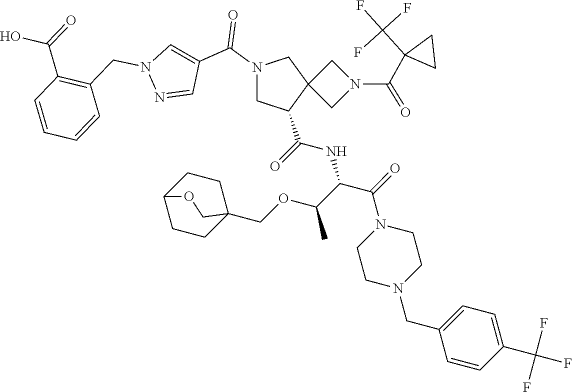 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)C1(C(F)(F)F)CC1)C2)C(=O)N1CCN(Cc2ccc(C(F)(F)F)cc2)CC1] |
| I-15 | 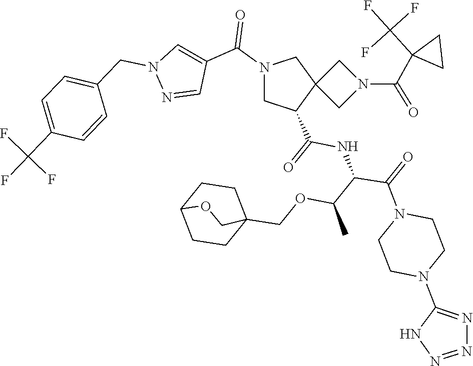 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCN(c2nnn[nH]2)CC1] |
| I-16 | 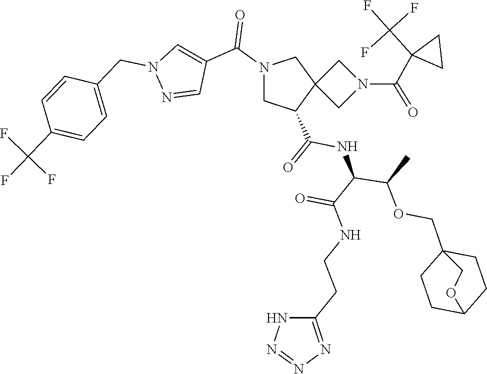 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)NCCc1nnn[nH]1] |
| I-17 | 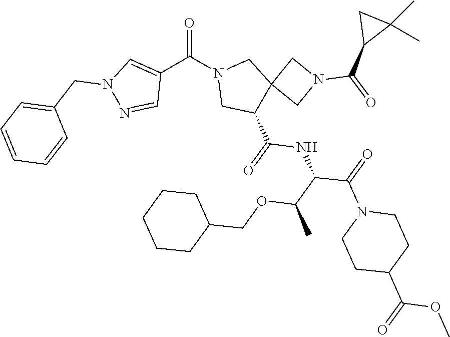 [SMILES: COC(=O)C1CCN(C(=O)[C@@H](NC(=O)[C@@H]2CN(C(=O)c3cnn(Cc4ccccc4)c3)CC23CN(C(=O)[C@H]2CC2(C)C)C3)[C@@H](C)OCC2CCCCC2)CC1] |
| I-18 | 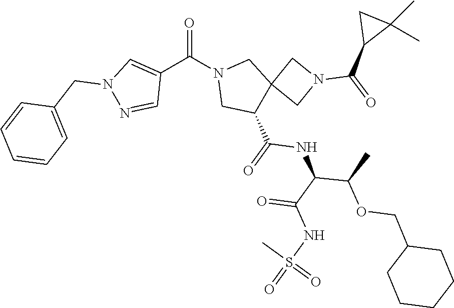 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)NS(C)(=O)=O] |
| I-19 | 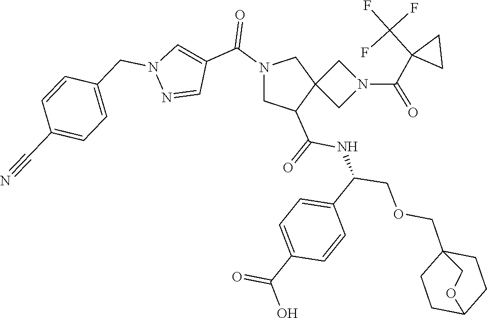 [SMILES: N#Cc1ccc(Cn2cc(C(=O)N3CC(C(=O)N[C@H](COCC45CCC(CC4)OC5)c4ccc(C(=O)O)cc4)C4(C3)CN(C(=O)C3(C(F)(F)F)CC3)C4)cn2)cc1] |
| I-20 | 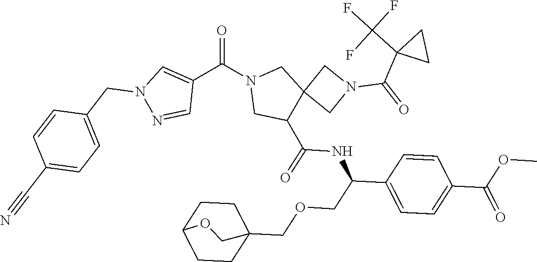 [SMILES: COC(=O)c1ccc([C@@H](COCC23CCC(CC2)OC3)NC(=O)C2CN(C(=O)c3cnn(Cc4ccc(C#N)cc4)c3)CC23CN(C(=O)C2(C(F)(F)F)CC2)C3)cc1] |
| I-21 | 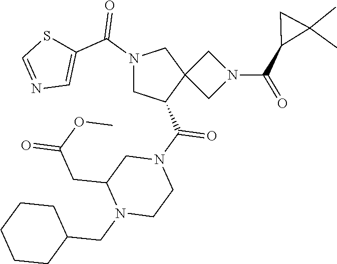 [SMILES: COC(=O)CC1CN(C(=O)[C@@H]2CN(C(=O)c3cncs3)CC23CN(C(=O)[C@H]2CC2(C)C)C3)CCN1CC1CCCCC1] |
| I-22 | 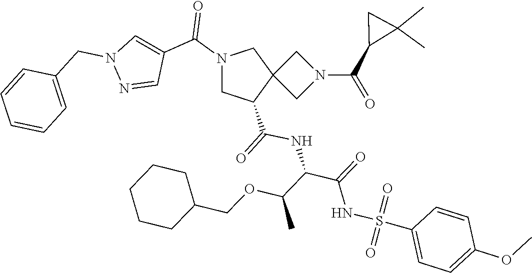 [SMILES: COc1ccc(S(=O)(=O)NC(=O)[C@@H](NC(=O)[C@@H]2CN(C(=O)c3cnn(Cc4ccccc4)c3)CC23CN(C(=O)[C@H]2CC2(C)C)C3)[C@@H](C)OCC2CCCCC2)cc1] |
| I-23 | 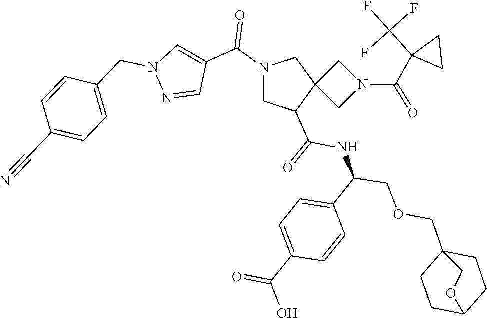 [SMILES: N#Cc1ccc(Cn2cc(C(=O)N3CC(C(=O)N[C@@H](COCC45CCC(CC4)OC5)c4ccc(C(=O)O)cc4)C4(C3)CN(C(=O)C3(C(F)(F)F)CC3)C4)cn2)cc1] |
| I-24 | 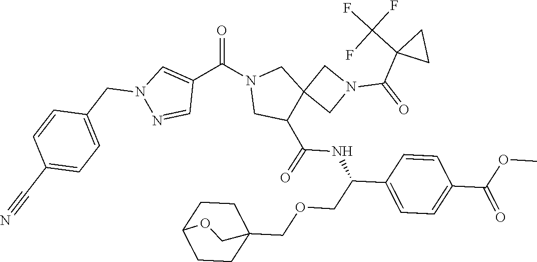 [SMILES: COC(=O)c1ccc([C@H](COCC23CCC(CC2)OC3)NC(=O)C2CN(C(=O)c3cnn(Cc4ccc(C#N)cc4)c3)CC23CN(C(=O)C2(C(F)(F)F)CC2)C3)cc1] |
| I-25 | 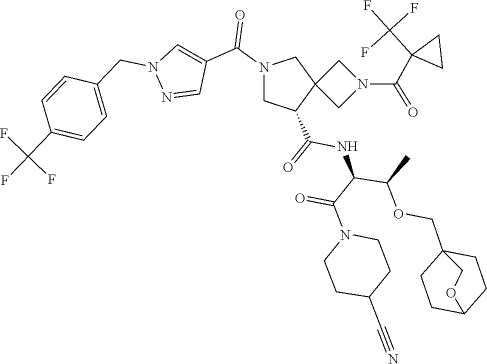 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(C#N)CC1] |
| I-26 | 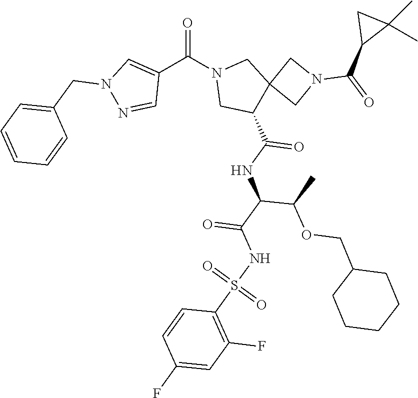 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)NS(=O)(=O)c1ccc(F)cc1F] |
| I-27 | 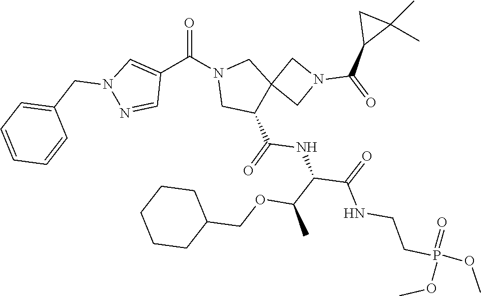 [SMILES: COP(=O)(CCNC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1)OC] |
| I-28 | 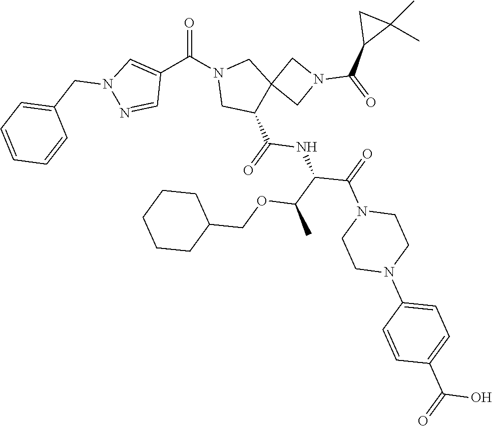 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCN(c2ccc(C(=O)O)cc2)CC1] |
| I-29 | 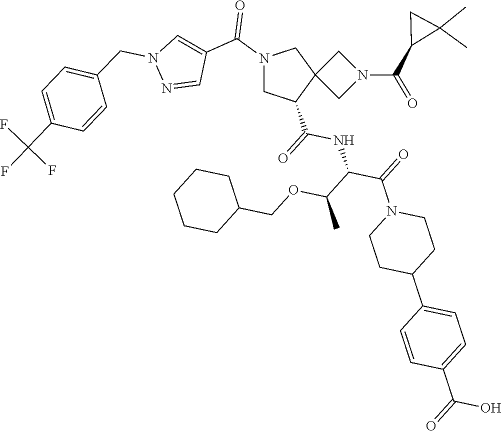 [SMILES: C[C@@H](OCC1CCCCC1)[C@H](NC(=O)[C@@H]1CN(C(=O)c2cnn(Cc3ccc(C(F)(F)F)cc3)c2)CC12CN(C(=O)[C@H]1CC1(C)C)C2)C(=O)N1CCC(c2ccc(C(=O)O)cc2)CC1] |
| I-30 | 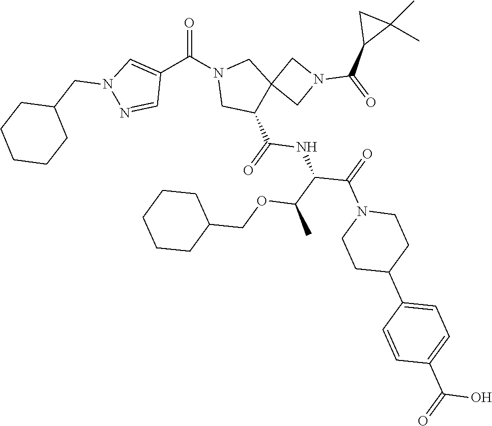 [SMILES: C[C@@H](OCC1CCCCC1)[C@H](NC(=O)[C@@H]1CN(C(=O)c2cnn(CC3CCCCC3)c2)CC12CN(C(=O)[C@H]1CC1(C)C)C2)C(=O)N1CCC(c2ccc(C(=O)O)cc2)CC1] |
| I-31 | 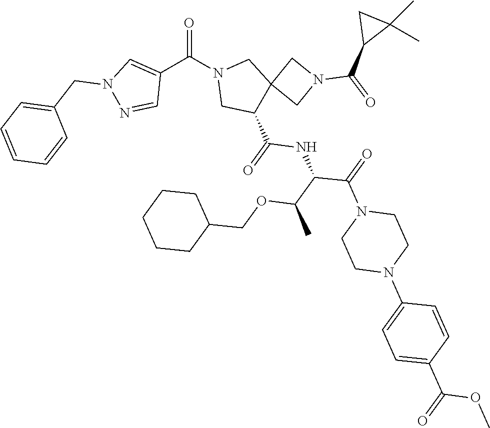 [SMILES: COC(=O)c1ccc(N2CCN(C(=O)[C@@H](NC(=O)[C@@H]3CN(C(=O)c4cnn(Cc5ccccc5)c4)CC34CN(C(=O)[C@H]3CC3(C)C)C4)[C@@H](C)OCC3CCCCC3)CC2)cc1] |
| I-32 | 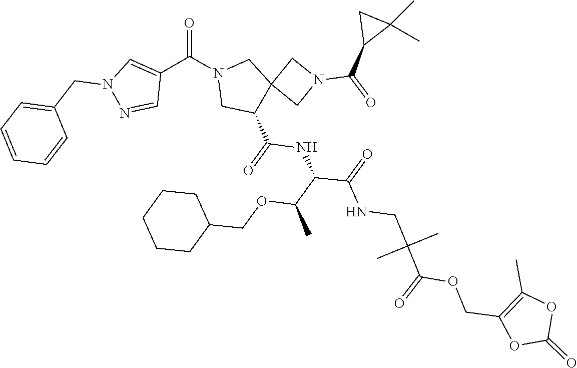 [SMILES: Cc1oc(=O)oc1COC(=O)C(C)(C)CNC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1] |
| I-33 | 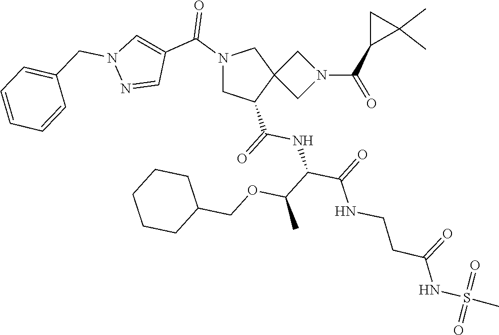 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)NCCC(=O)NS(C)(=O)=O] |
| I-34 | 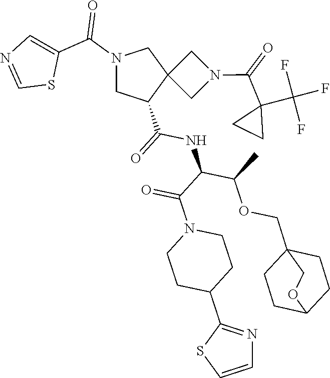 [SMILES: C[C@@H](OCC12CCC(CC1)OC2)[C@H](NC(=O)[C@@H]1CN(C(=O)c2cncs2)CC12CN(C(=O)C1(C(F)(F)F)CC1)C2)C(=O)N1CCC(c2nccs2)CC1] |
| I-35 | 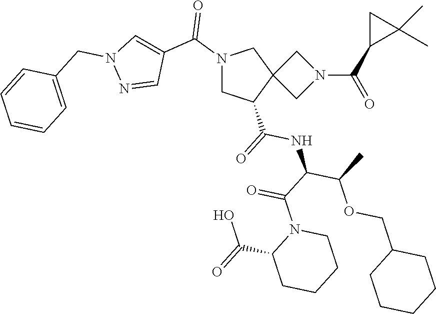 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCCC[C@@H]1C(=O)O] |
| I-36 | 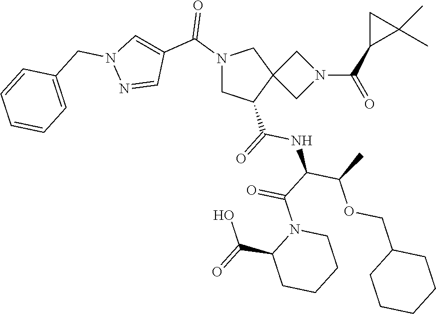 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCCC[C@H]1C(=O)O] |
| I-37 | 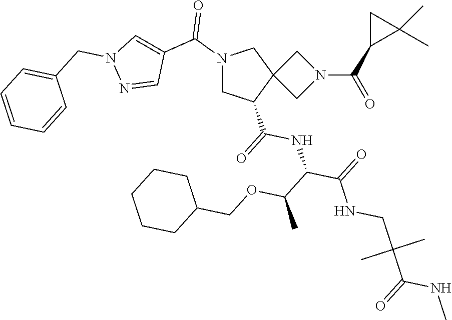 [SMILES: CNC(=O)C(C)(C)CNC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1] |
| I-38 | 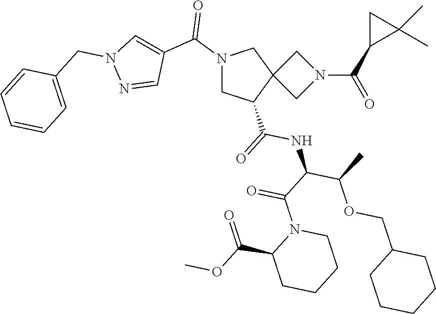 [SMILES: COC(=O)[C@@H]1CCCCN1C(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1] |
| I-39 | 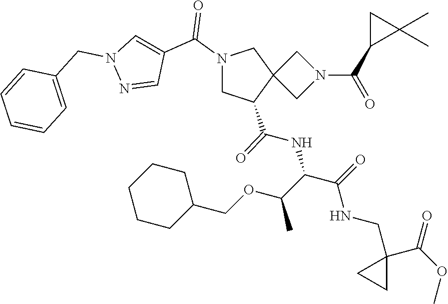 [SMILES: COC(=O)C1(CNC(=O)[C@@H](NC(=O)[C@@H]2CN(C(=O)c3cnn(Cc4ccccc4)c3)CC23CN(C(=O)[C@H]2CC2(C)C)C3)[C@@H](C)OCC2CCCCC2)CC1] |
| I-40 | 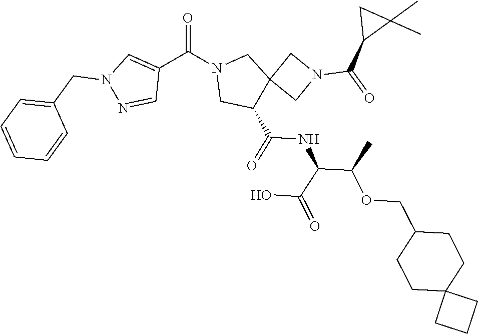 [SMILES: C[C@@H](OCC1CCC2(CCC2)CC1)[C@H](NC(=O)[C@@H]1CN(C(=O)c2cnn(Cc3ccccc3)c2)CC12CN(C(=O)[C@H]1CC1(C)C)C2)C(=O)O] |
| I-41 | 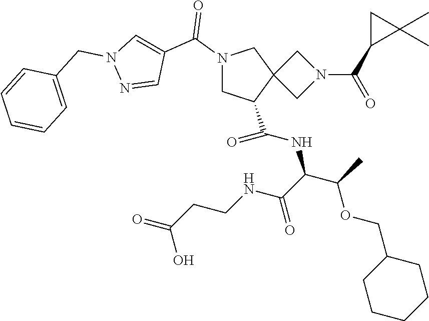 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)NCCC(=O)O] |
| I-42 | 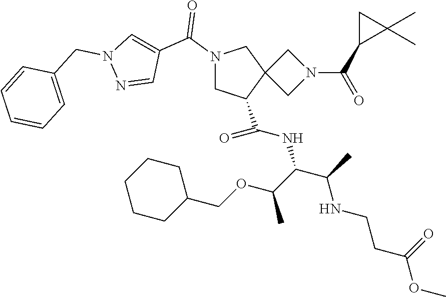 [SMILES: COC(=O)CCN[C@H](C)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1] |
| I-43 | 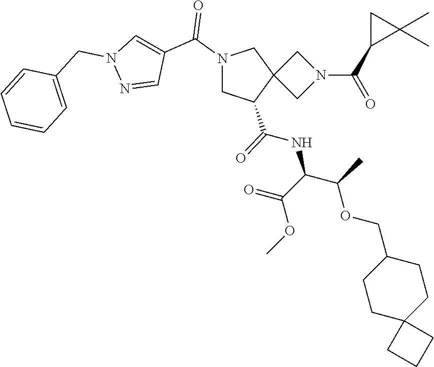 [SMILES: COC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCC2(CCC2)CC1] |
| I-44 | 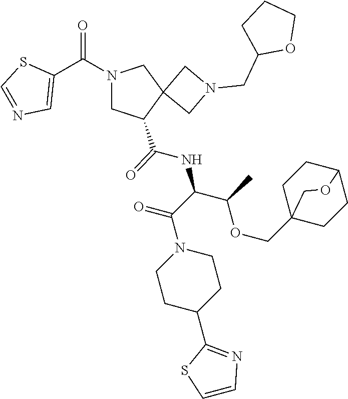 [SMILES: C[C@@H](OCC12CCC(CC1)OC2)[C@H](NC(=O)[C@@H]1CN(C(=O)c2cncs2)CC12CN(CC1CCCO1)C2)C(=O)N1CCC(c2nccs2)CC1] |
| I-45 | 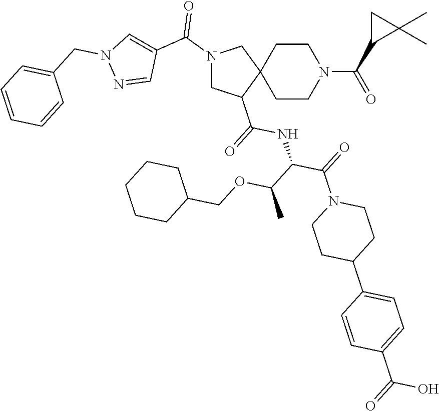 |
| I-46 | 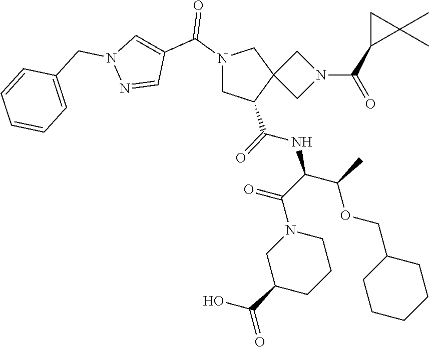 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCC[C@@H](C(=O)O)C1] |
| I-47 | 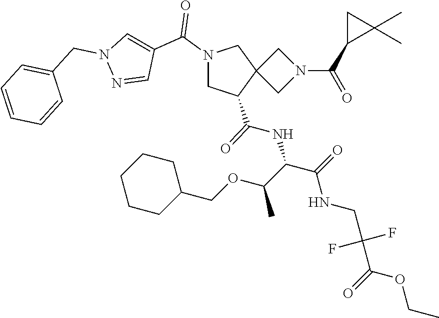 [SMILES: CCOC(=O)C(F)(F)CNC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1] |
| I-48 | 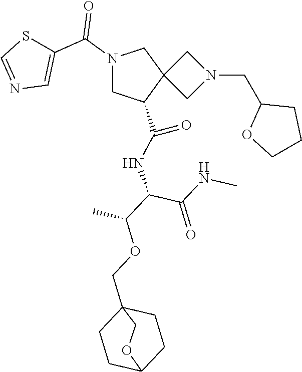 [SMILES: CNC(=O)[C@@H](NC(=O)[C@@H]1CN(C(=O)c2cncs2)CC12CN(CC1CCCO1)C2)[C@@H](C)OCC12CCC(CC1)OC2] |
| I-49 | 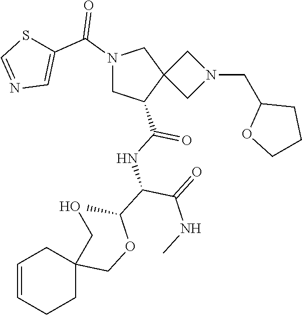 [SMILES: CNC(=O)[C@@H](NC(=O)[C@@H]1CN(C(=O)c2cncs2)CC12CN(CC1CCCO1)C2)[C@@H](C)OCC1(CO)CC=CCC1] |
| I-50 | 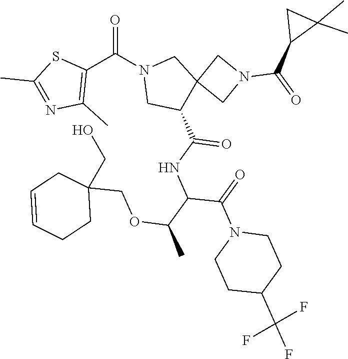 |
| I-51 | 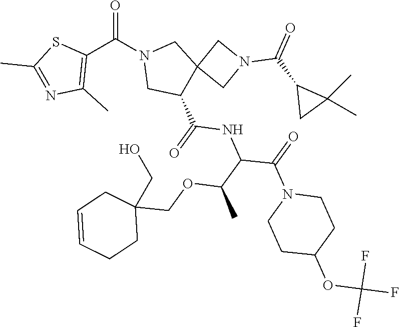 [SMILES: Cc1nc(C)c(C(=O)N2C[C@@H](C(=O)NC(C(=O)N3CCC(OC(F)(F)F)CC3)[C@@H](C)OCC3(CO)CC=CCC3)C3(C2)CN(C(=O)[C@H]2CC2(C)C)C3)s1] |
| I-52 | 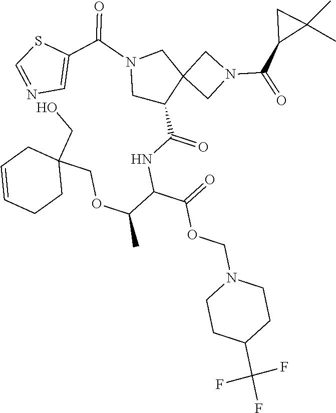 [SMILES: C[C@@H](OCC1(CO)CC=CCC1)C(NC(=O)[C@@H]1CN(C(=O)c2cncs2)CC12CN(C(=O)[C@H]1CC1(C)C)C2)C(=O)OCN1CCC(C(F)(F)F)CC1] |
| I-53 | 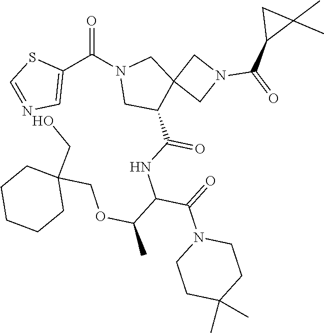 [SMILES: C[C@@H](OCC1(CO)CCCCC1)C(NC(=O)[C@@H]1CN(C(=O)c2cncs2)CC12CN(C(=O)[C@H]1CC1(C)C)C2)C(=O)N1CCC(C)(C)CC1] |
| I-54 | 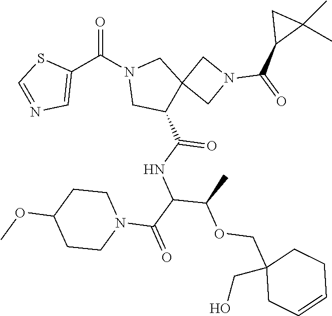 |
| I-55 | 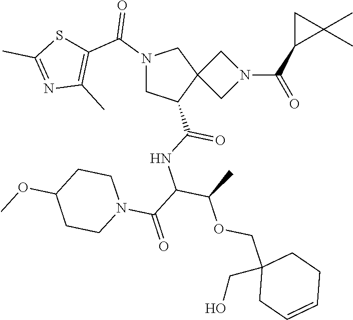 |
| I-56 | 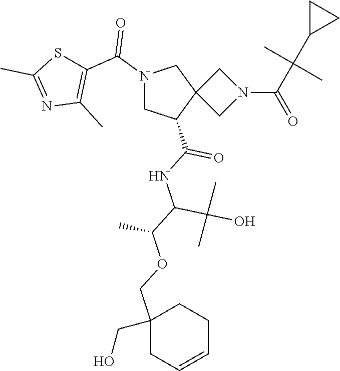 [SMILES: Cc1nc(C)c(C(=O)N2C[C@@H](C(=O)NC([C@@H](C)OCC3(CO)CC=CCC3)C(C)(C)O)C3(C2)CN(C(=O)C(C)(C)C2CC2)C3)s1] |
| I-57 | 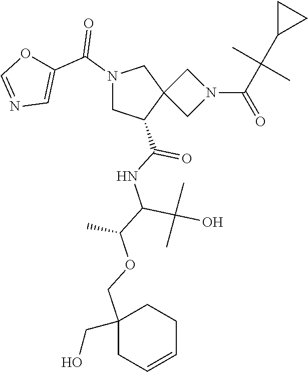 [SMILES: C[C@@H](OCC1(CO)CC=CCC1)C(NC(=O)[C@@H]1CN(C(=O)c2cnco2)CC12CN(C(=O)C(C)(C)C1CC1)C2)C(C)(C)O] |
| I-58 | 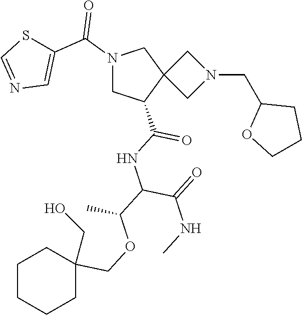 [SMILES: CNC(=O)C(NC(=O)[C@@H]1CN(C(=O)c2cncs2)CC12CN(CC1CCCO1)C2)[C@@H](C)OCC1(CO)CCCCC1] |
| I-59 | 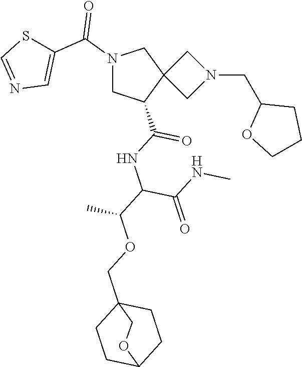 [SMILES: CNC(=O)C(NC(=O)[C@@H]1CN(C(=O)c2cncs2)CC12CN(CC1CCCO1)C2)[C@@H](C)OCC12CCC(CC1)OC2] |
| I-60 | 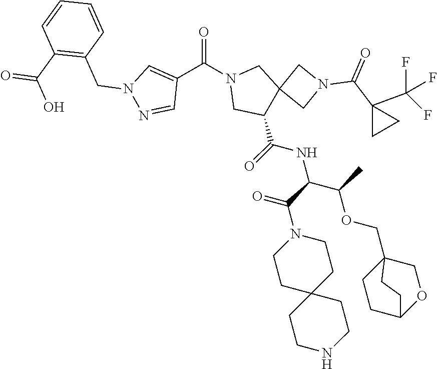 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)C1(C(F)(F)F)CC1)C2)C(=O)N1CCC2(CCNCC2)CC1] |
| I-61 | 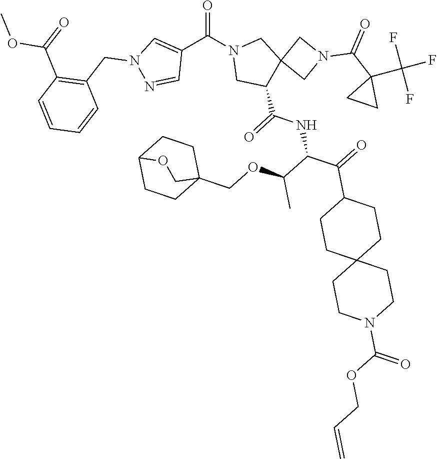 [SMILES: C=CCOC(=O)N1CCC2(CCC(C(=O)[C@@H](NC(=O)[C@@H]3CN(C(=O)c4cnn(Cc5ccccc5C(=O)OC)c4)CC34CN(C(=O)C3(C(F)(F)F)CC3)C4)[C@@H](C)OCC34CCC(CC3)OC4)CC2)CC1] |
| I-62 | 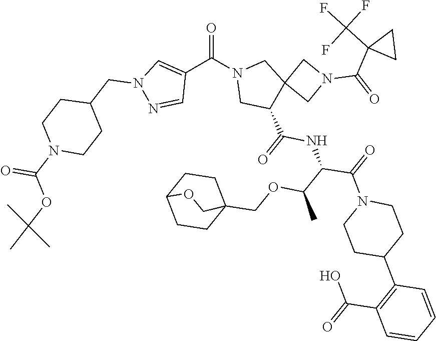 [SMILES: C[C@@H](OCC12CCC(CC1)OC2)[C@H](NC(=O)[C@@H]1CN(C(=O)c2cnn(CC3CCN(C(=O)OC(C)(C)C)CC3)c2)CC12CN(C(=O)C1(C(F)(F)F)CC1)C2)C(=O)N1CCC(c2ccccc2C(=O)O)CC1] |
| I-63 | 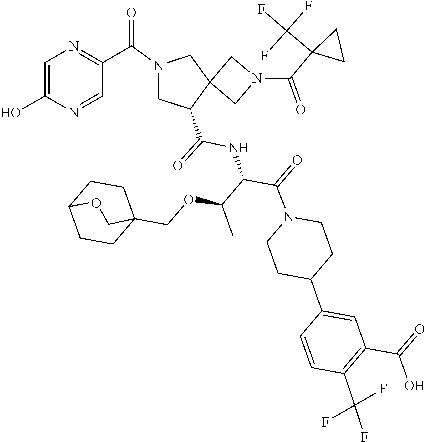 [SMILES: C[C@@H](OCC12CCC(CC1)OC2)[C@H](NC(=O)[C@@H]1CN(C(=O)c2cnc(O)cn2)CC12CN(C(=O)C1(C(F)(F)F)CC1)C2)C(=O)N1CCC(c2ccc(C(F)(F)F)c(C(=O)O)c2)CC1] |
| I-64 | 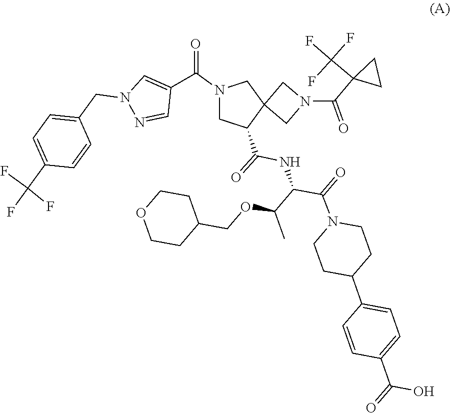 [SMILES: C[C@@H](OCC1CCOCC1)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccc(C(=O)O)cc2)CC1] |
| | 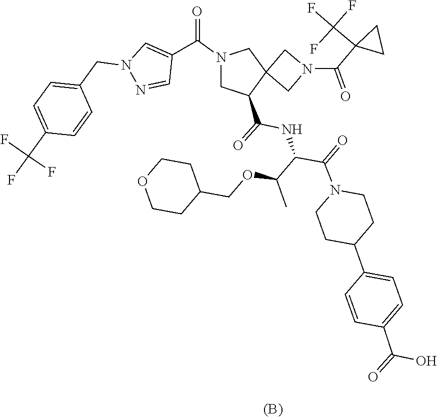 [SMILES: C[C@@H](OCC1CCOCC1)[C@H](NC(=O)[C@H]1CN(C(=O)c2cnn(Cc3ccc(C(F)(F)F)cc3)c2)CC12CN(C(=O)C1(C(F)(F)F)CC1)C2)C(=O)N1CCC(c2ccc(C(=O)O)cc2)CC1 ; "(B)"] |
| I-65 | 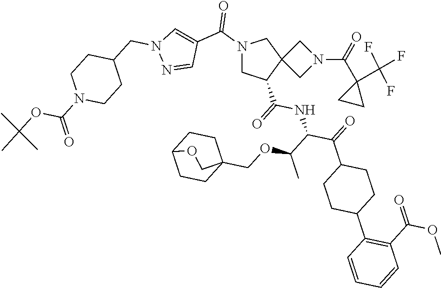 [SMILES: COC(=O)c1ccccc1C1CCC(C(=O)[C@@H](NC(=O)[C@@H]2CN(C(=O)c3cnn(CC4CCN(C(=O)OC(C)(C)C)CC4)c3)CC23CN(C(=O)C2(C(F)(F)F)CC2)C3)[C@@H](C)OCC23CCC(CC2)OC3)CC1] |
| I-66 | 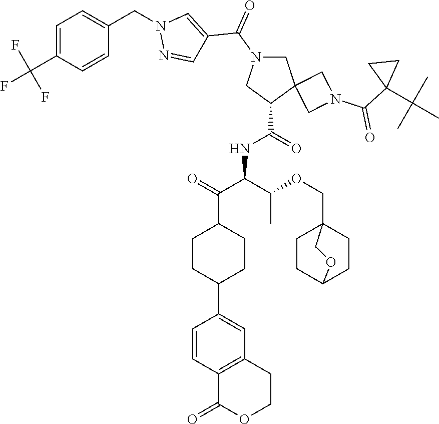 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(C)(C)C)CC1)C2)C(=O)C1CCC(c2ccc3c(c2)CCOC3=O)CC1] |
| I-67 | 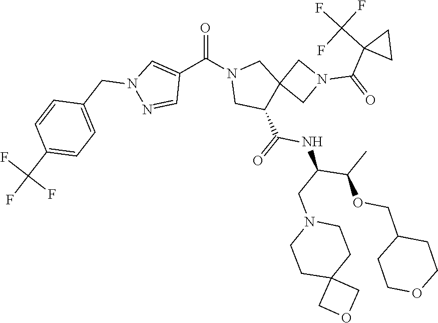 [SMILES: C[C@@H](OCC1CCOCC1)[C@@H](CN1CCC2(CC1)COC2)NC(=O)[C@@H]1CN(C(=O)c2cnn(Cc3ccc(C(F)(F)F)cc3)c2)CC12CN(C(=O)C1(C(F)(F)F)CC1)C2] |
| I-68 | 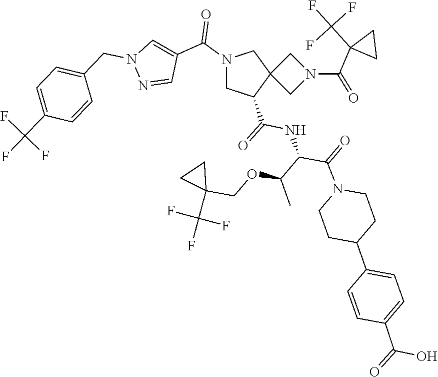 [SMILES: C[C@@H](OCC1(C(F)(F)F)CC1)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccc(C(=O)O)cc2)CC1] |
| I-69 | 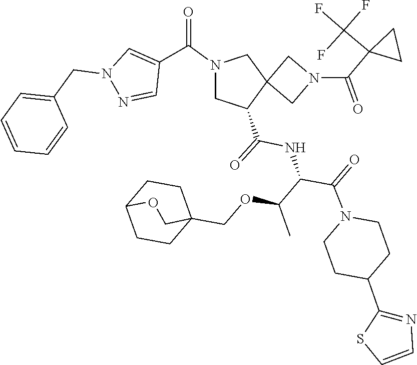 [SMILES: C[C@@H](OCC12CCC(CC1)OC2)[C@H](NC(=O)[C@@H]1CN(C(=O)c2cnn(Cc3ccccc3)c2)CC12CN(C(=O)C1(C(F)(F)F)CC1)C2)C(=O)N1CCC(c2nccs2)CC1] |
| I-70 | 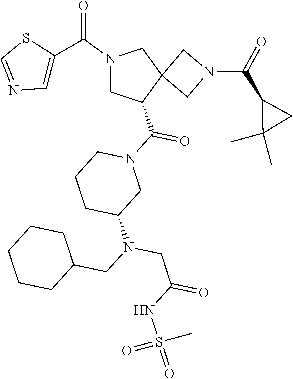 [SMILES: CC1(C)C[C@@H]1C(=O)N1CC2(CN(C(=O)c3cncs3)C[C@H]2C(=O)N2CCC[C@@H](N(CC(=O)NS(C)(=O)=O)CC3CCCCC3)C2)C1] |
| I-71 | 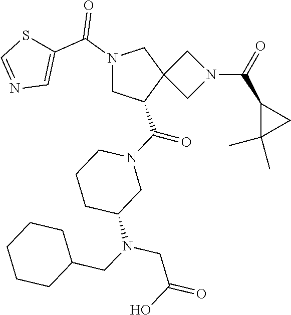 [SMILES: CC1(C)C[C@@H]1C(=O)N1CC2(CN(C(=O)c3cncs3)C[C@H]2C(=O)N2CCC[C@@H](N(CC(=O)O)CC3CCCCC3)C2)C1] |
| I-72 | 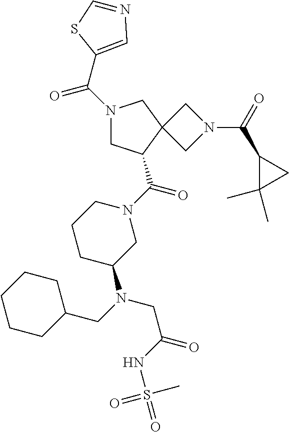 [SMILES: CC1(C)C[C@@H]1C(=O)N1CC2(CN(C(=O)c3cncs3)C[C@H]2C(=O)N2CCC[C@H](N(CC(=O)NS(C)(=O)=O)CC3CCCCC3)C2)C1] |
| I-73 | 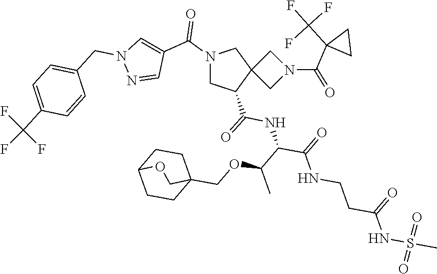 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)NCCC(=O)NS(C)(=O)=O] |
| I-74 | 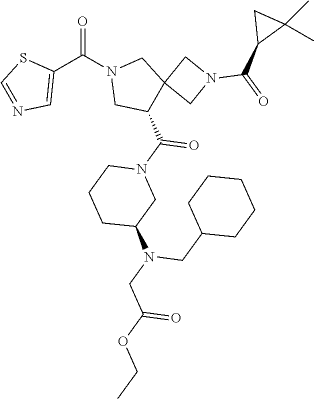 [SMILES: CCOC(=O)CN(CC1CCCCC1)[C@H]1CCCN(C(=O)[C@@H]2CN(C(=O)c3cncs3)CC23CN(C(=O)[C@H]2CC2(C)C)C3)C1] |
| I-75 | 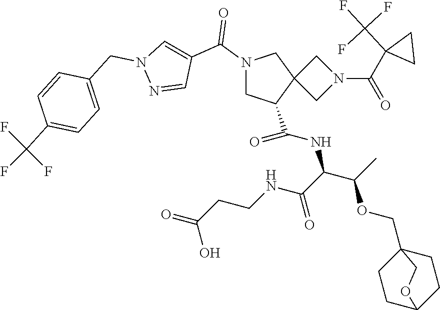 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)NCCC(=O)O] |
| I-76 | 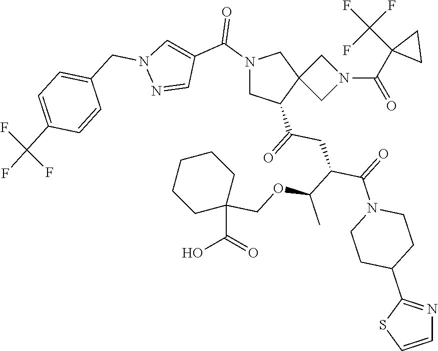 [SMILES: C[C@@H](OCC1(C(=O)O)CCCCC1)[C@H](CC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2nccs2)CC1] |
| I-77 | 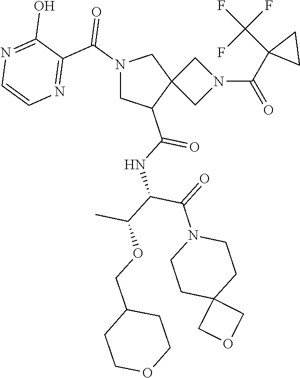 [SMILES: C[C@@H](OCC1CCOCC1)[C@H](NC(=O)C1CN(C(=O)c2nccnc2O)CC12CN(C(=O)C1(C(F)(F)F)CC1)C2)C(=O)N1CCC2(CC1)COC2] |
| I-78 | 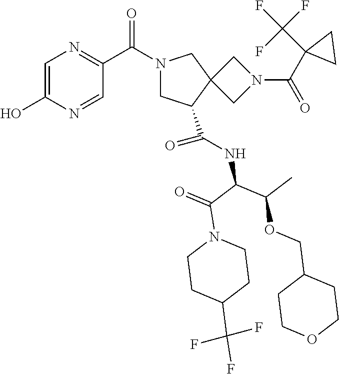 [SMILES: C[C@@H](OCC1CCOCC1)[C@H](NC(=O)[C@@H]1CN(C(=O)c2cnc(O)cn2)CC12CN(C(=O)C1(C(F)(F)F)CC1)C2)C(=O)N1CCC(C(F)(F)F)CC1] |
| I-79 | 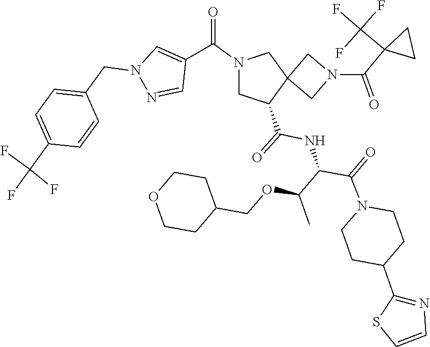 [SMILES: C[C@@H](OCC1CCOCC1)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2nccs2)CC1] |
| I-80 | 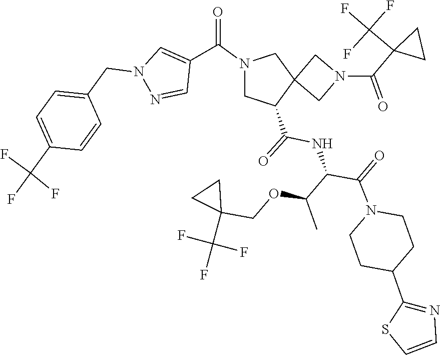 [SMILES: C[C@@H](OCC1(C(F)(F)F)CC1)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2nccs2)CC1] |
| I-81 | 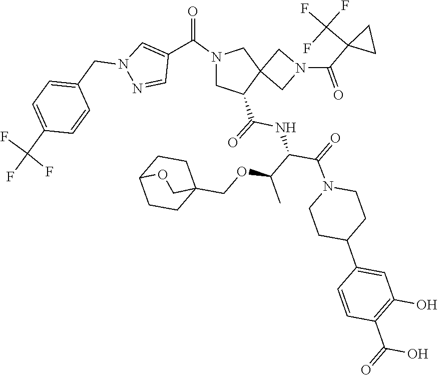 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccc(C(=O)O)c(O)c2)CC1] |
| I-82 | 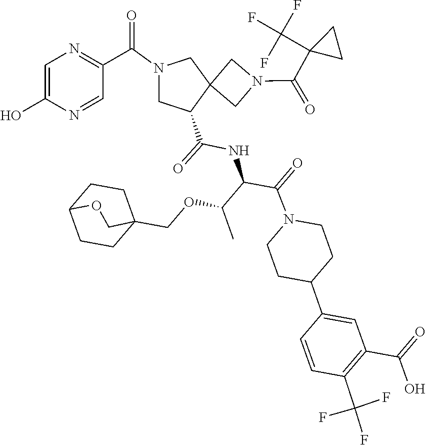 [SMILES: C[C@H](OCC12CCC(CC1)OC2)[C@@H](NC(=O)[C@@H]1CN(C(=O)c2cnc(O)cn2)CC12CN(C(=O)C1(C(F)(F)F)CC1)C2)C(=O)N1CCC(c2ccc(C(F)(F)F)c(C(=O)O)c2)CC1] |
| I-83 | 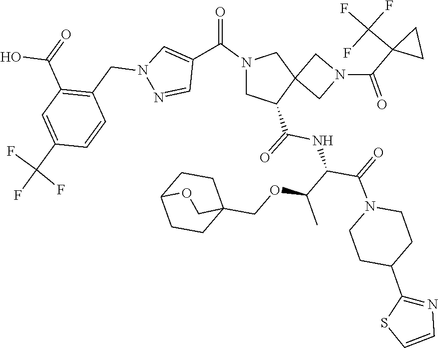 [SMILES: C[C@@H](OCC12CCC(CC1)OC2)[C@H](NC(=O)[C@@H]1CN(C(=O)c2cnn(Cc3ccc(C(F)(F)F)cc3C(=O)O)c2)CC12CN(C(=O)C1(C(F)(F)F)CC1)C2)C(=O)N1CCC(c2nccs2)CC1] |
| I-84 | 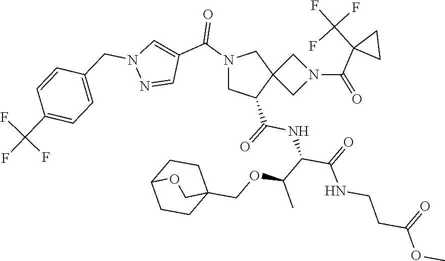 [SMILES: COC(=O)CCNC(=O)[C@@H](NC(=O)[C@@H]1CN(C(=O)c2cnn(Cc3ccc(C(F)(F)F)cc3)c2)CC12CN(C(=O)C1(C(F)(F)F)CC1)C2)[C@@H](C)OCC12CCC(CC1)OC2] |
| I-85 | 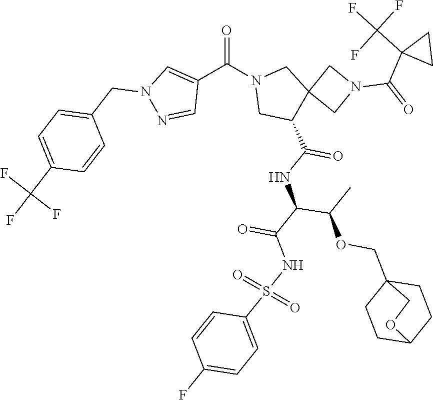 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)NS(=O)(=O)c1ccc(F)cc1] |
| I-86 | 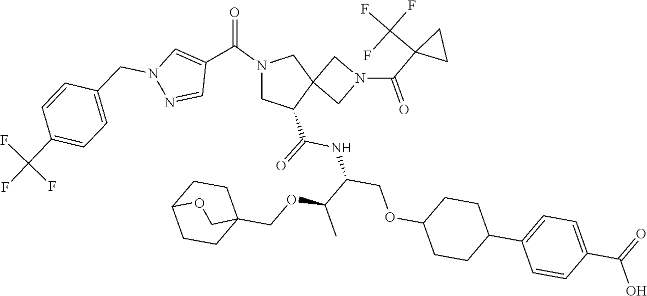 [SMILES: C[C@@H](OCC12CCC(CC1)OC2)[C@@H](COC1CCC(c2ccc(C(=O)O)cc2)CC1)NC(=O)[C@@H]1CN(C(=O)c2cnn(Cc3ccc(C(F)(F)F)cc3)c2)CC12CN(C(=O)C1(C(F)(F)F)CC1)C2] |
| I-87 | 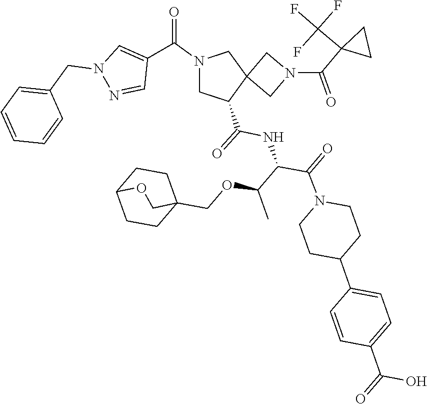 [SMILES: C[C@@H](OCC12CCC(CC1)OC2)[C@H](NC(=O)[C@@H]1CN(C(=O)c2cnn(Cc3ccccc3)c2)CC12CN(C(=O)C1(C(F)(F)F)CC1)C2)C(=O)N1CCC(c2ccc(C(=O)O)cc2)CC1] |
| I-88 | 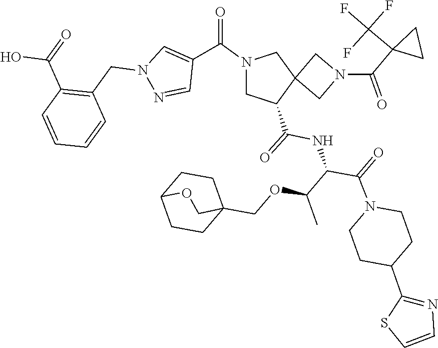 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)C1(C(F)(F)F)CC1)C2)C(=O)N1CCC(c2nccs2)CC1] |
| I-89 | 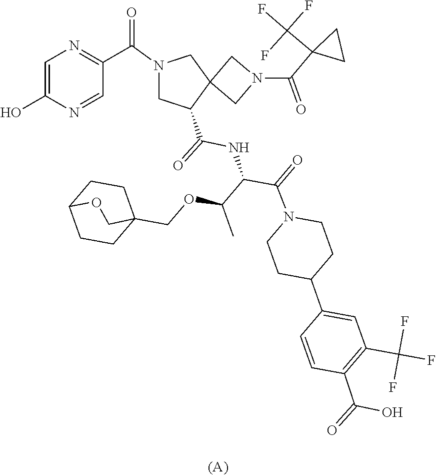 [SMILES: C[C@@H](OCC12CCC(CC1)OC2)[C@H](NC(=O)[C@@H]1CN(C(=O)c2cnc(O)cn2)CC12CN(C(=O)C1(C(F)(F)F)CC1)C2)C(=O)N1CCC(c2ccc(C(=O)O)c(C(F)(F)F)c2)CC1 ; "(A)"] |
| | 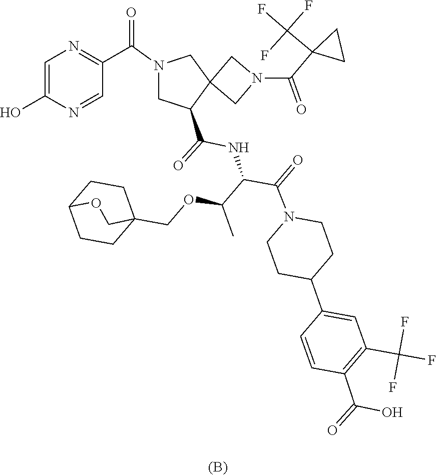 [SMILES: C[C@@H](OCC12CCC(CC1)OC2)[C@H](NC(=O)[C@H]1CN(C(=O)c2cnc(O)cn2)CC12CN(C(=O)C1(C(F)(F)F)CC1)C2)C(=O)N1CCC(c2ccc(C(=O)O)c(C(F)(F)F)c2)CC1 ; "(B)"] |
| | 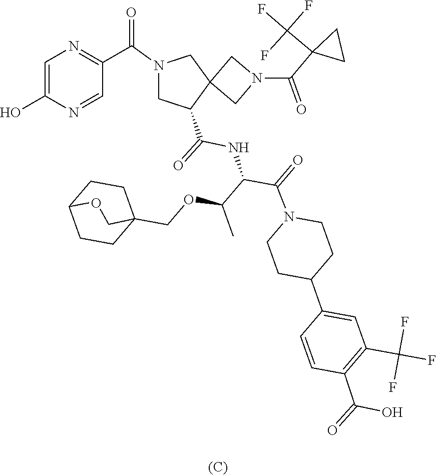 [SMILES: C[C@@H](OCC12CCC(CC1)OC2)[C@H](NC(=O)[C@@H]1CN(C(=O)c2cnc(O)cn2)CC12CN(C(=O)C1(C(F)(F)F)CC1)C2)C(=O)N1CCC(c2ccc(C(=O)O)c(C(F)(F)F)c2)CC1 ; "(C)"] |
| I-90 | 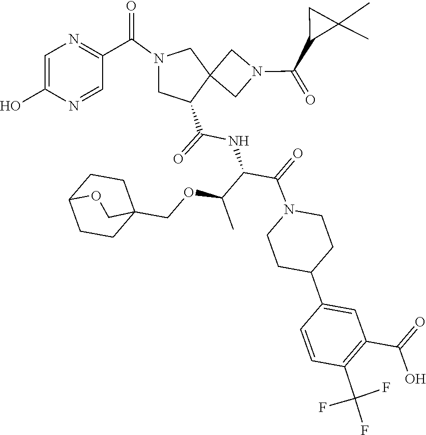 |
| I-91 | 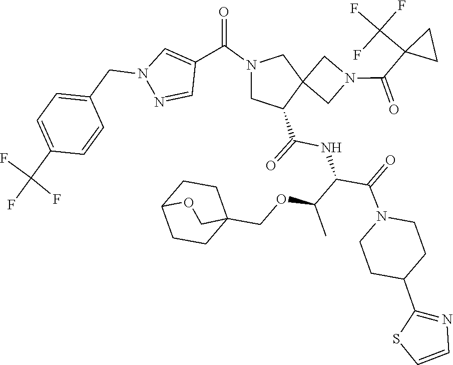 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2nccs2)CC1] |
| I-92 | 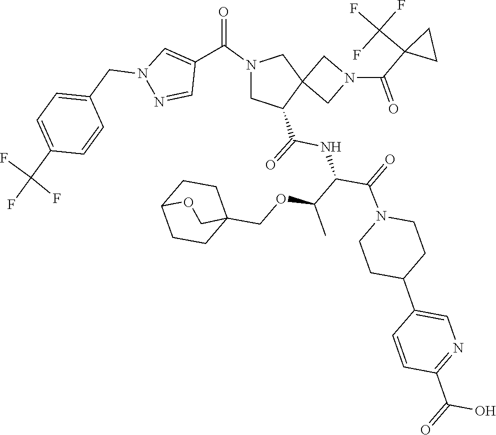 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccc(C(=O)O)nc2)CC1] |
| I-93 | 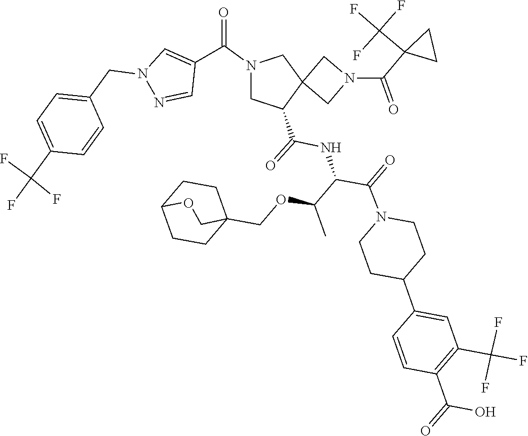 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccc(C(=O)O)c(C(F)(F)F)c2)CC1] |
| I-94 | 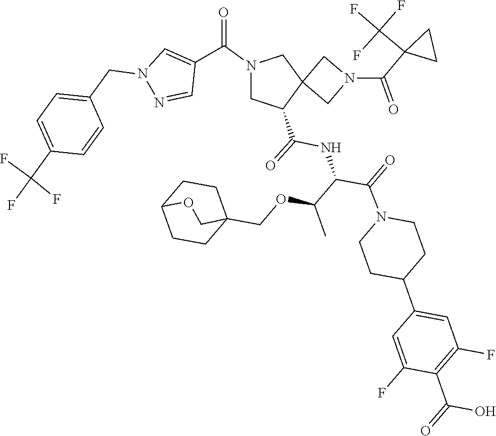 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2cc(F)c(C(=O)O)c(F)c2)CC1] |
| I-95 | 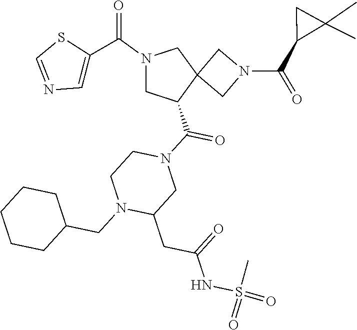 [SMILES: CC1(C)C[C@@H]1C(=O)N1CC2(CN(C(=O)c3cncs3)C[C@H]2C(=O)N2CCN(CC3CCCCC3)C(CC(=O)NS(C)(=O)=O)C2)C1] |
| I-96 | 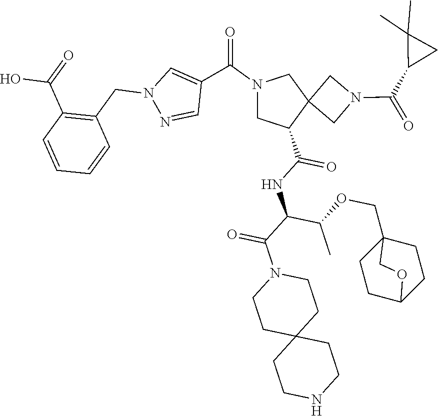 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)[C@H]1CC1(C)C)C2)C(=O)N1CCC2(CCNCC2)CC1] |
| I-97 | 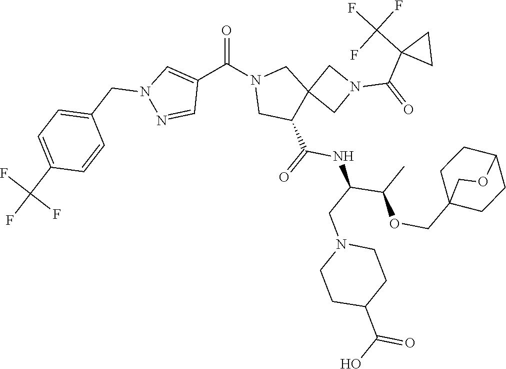 [SMILES: C[C@@H](OCC12CCC(CC1)OC2)[C@@H](CN1CCC(C(=O)O)CC1)NC(=O)[C@@H]1CN(C(=O)c2cnn(Cc3ccc(C(F)(F)F)cc3)c2)CC12CN(C(=O)C1(C(F)(F)F)CC1)C2] |
| I-98 | 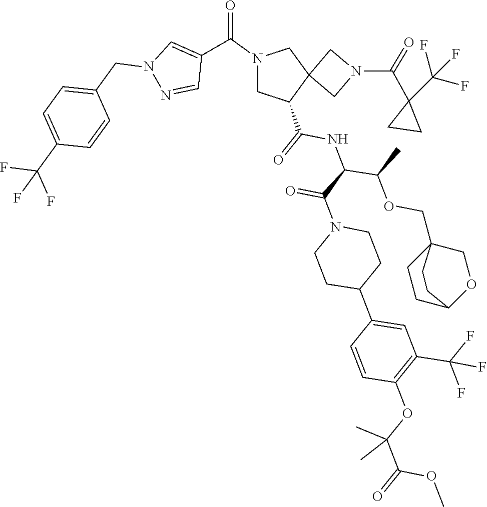 [SMILES: COC(=O)C(C)(C)Oc1ccc(C2CCN(C(=O)[C@@H](NC(=O)[C@@H]3CN(C(=O)c4cnn(Cc5ccc(C(F)(F)F)cc5)c4)CC34CN(C(=O)C3(C(F)(F)F)CC3)C4)[C@@H](C)OCC34CCC(CC3)OC4)CC2)cc1C(F)(F)F] |
| I-99 | 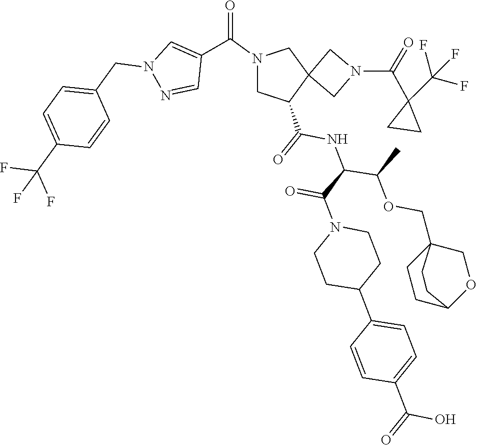 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccc(C(=O)O)cc2)CC1] |
| I-100 | 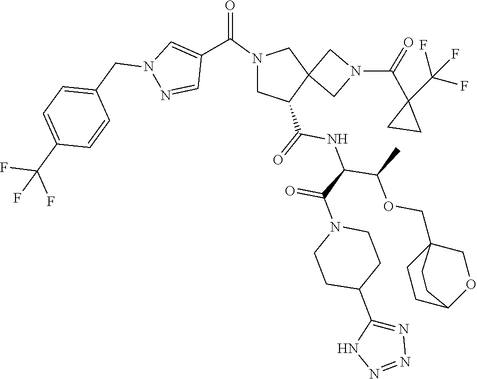 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2nnn[nH]2)CC1] |
| I-101 | 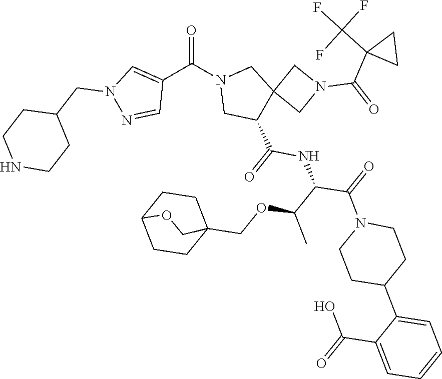 [SMILES: C[C@@H](OCC12CCC(CC1)OC2)[C@H](NC(=O)[C@@H]1CN(C(=O)c2cnn(CC3CCNCC3)c2)CC12CN(C(=O)C1(C(F)(F)F)CC1)C2)C(=O)N1CCC(c2ccccc2C(=O)O)CC1] |
| I-102 | 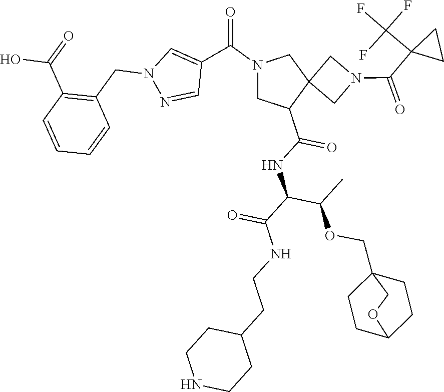 [SMILES: C[C@@H](OCC12CCC(CC1)OC2)[C@H](NC(=O)C1CN(C(=O)c2cnn(Cc3ccccc3C(=O)O)c2)CC12CN(C(=O)C1(C(F)(F)F)CC1)C2)C(=O)NCCC1CCNCC1] |
| I-103 | 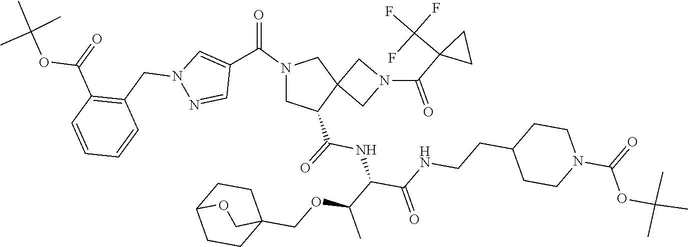 [SMILES: C[C@@H](OCC12CCC(CC1)OC2)[C@H](NC(=O)[C@@H]1CN(C(=O)c2cnn(Cc3ccccc3C(=O)OC(C)(C)C)c2)CC12CN(C(=O)C1(C(F)(F)F)CC1)C2)C(=O)NCCC1CCN(C(=O)OC(C)(C)C)CC1] |
| I-104 | 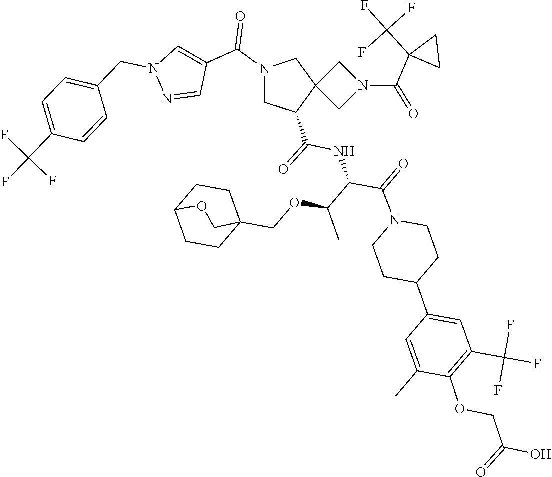 [SMILES: Cc1cc(C2CCN(C(=O)[C@@H](NC(=O)[C@@H]3CN(C(=O)c4cnn(Cc5ccc(C(F)(F)F)cc5)c4)CC34CN(C(=O)C3(C(F)(F)F)CC3)C4)[C@@H](C)OCC34CCC(CC3)OC4)CC2)cc(C(F)(F)F)c1OCC(=O)O] |
| I-105 | 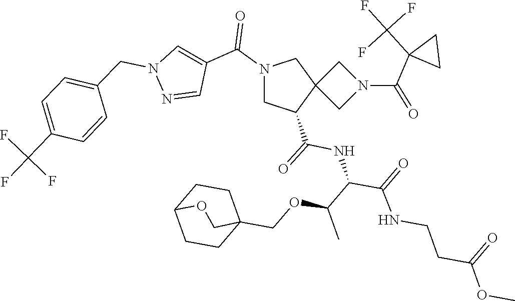 [SMILES: COC(=O)CCNC(=O)[C@@H](NC(=O)[C@@H]1CN(C(=O)c2cnn(Cc3ccc(C(F)(F)F)cc3)c2)CC12CN(C(=O)C1(C(F)(F)F)CC1)C2)[C@@H](C)OCC12CCC(CC1)OC2] |
| I-106 | 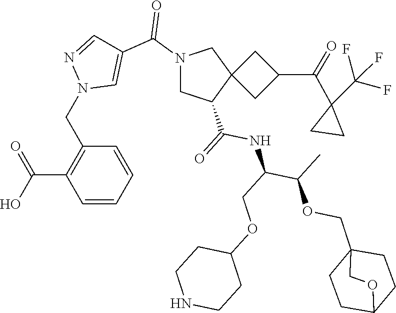 [SMILES: C[C@@H](OCC12CCC(CC1)OC2)[C@@H](COC1CCNCC1)NC(=O)[C@@H]1CN(C(=O)c2cnn(Cc3ccccc3C(=O)O)c2)CC12CC(C(=O)C1(C(F)(F)F)CC1)C2] |
| I-107 | 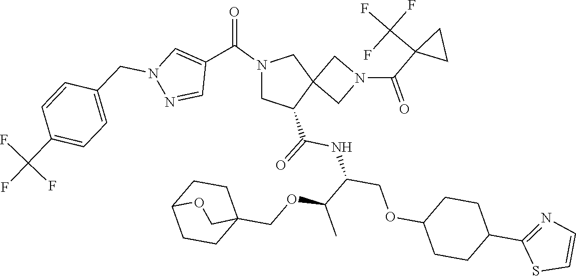 [SMILES: C[C@@H](OCC12CCC(CC1)OC2)[C@@H](COC1CCC(c2nccs2)CC1)NC(=O)[C@@H]1CN(C(=O)c2cnn(Cc3ccc(C(F)(F)F)cc3)c2)CC12CN(C(=O)C1(C(F)(F)F)CC1)C2] |
| I-108 | 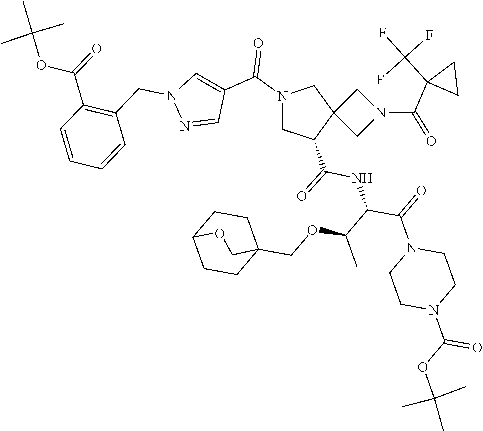 [SMILES: C[C@@H](OCC12CCC(CC1)OC2)[C@H](NC(=O)[C@@H]1CN(C(=O)c2cnn(Cc3ccccc3C(=O)OC(C)(C)C)c2)CC12CN(C(=O)C1(C(F)(F)F)CC1)C2)C(=O)N1CCN(C(=O)OC(C)(C)C)CC1] |
| I-109 | 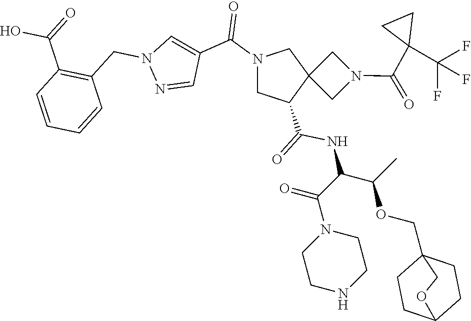 |
| I-110 | 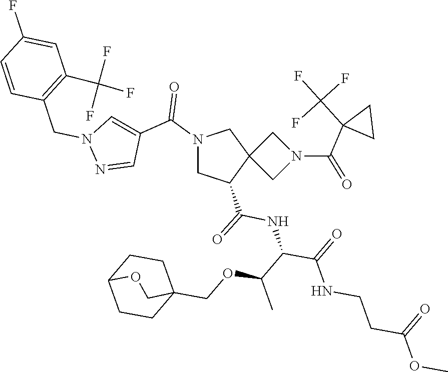 [SMILES: COC(=O)CCNC(=O)[C@@H](NC(=O)[C@@H]1CN(C(=O)c2cnn(Cc3ccc(F)cc3C(F)(F)F)c2)CC12CN(C(=O)C1(C(F)(F)F)CC1)C2)[C@@H](C)OCC12CCC(CC1)OC2] |
| I-111 | 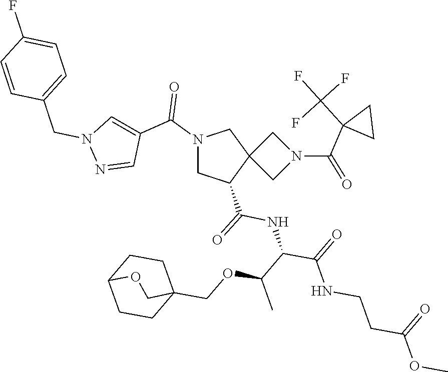 [SMILES: COC(=O)CCNC(=O)[C@@H](NC(=O)[C@@H]1CN(C(=O)c2cnn(Cc3ccc(F)cc3)c2)CC12CN(C(=O)C1(C(F)(F)F)CC1)C2)[C@@H](C)OCC12CCC(CC1)OC2] |
| I-112 | 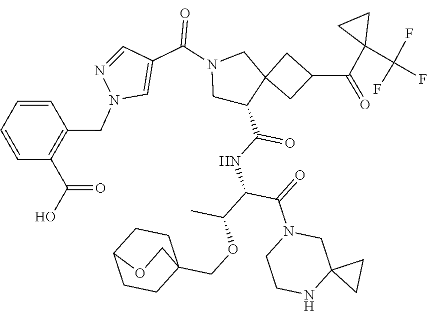 |
| I-113 | 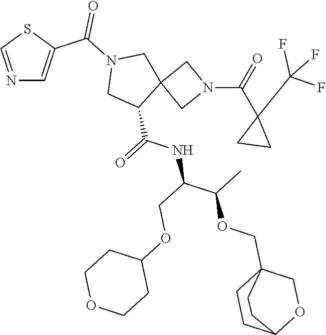 [SMILES: C[C@@H](OCC12CCC(CC1)OC2)[C@@H](COC1CCOCC1)NC(=O)[C@@H]1CN(C(=O)c2cncs2)CC12CN(C(=O)C1(C(F)(F)F)CC1)C2] |
| I-114 | 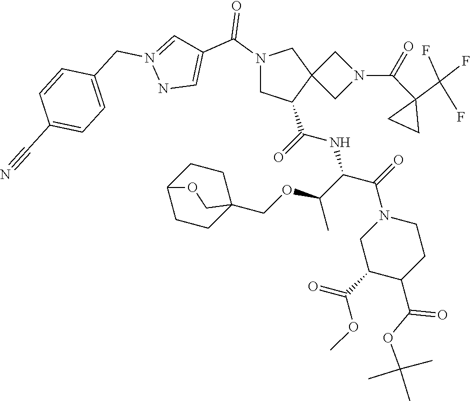 |
| I-115 | 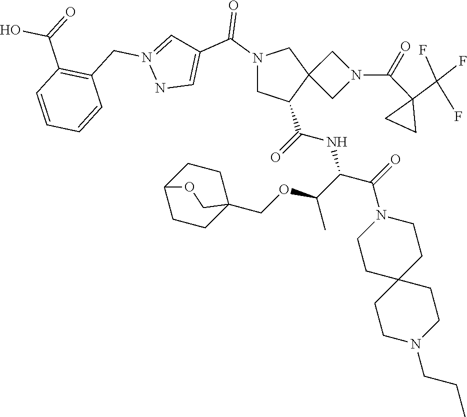 |
| I-116 | 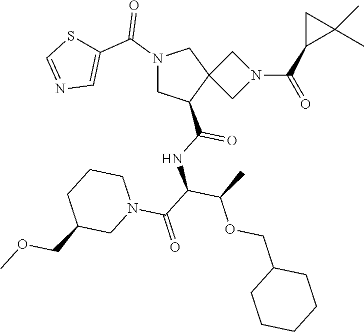 |
| I-117 | 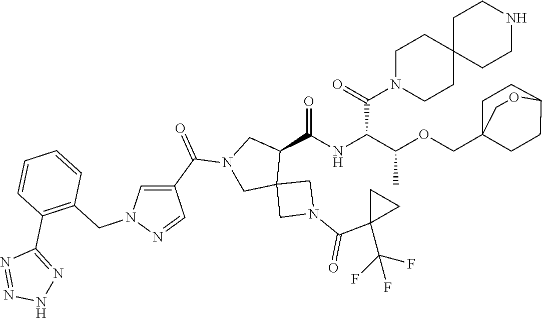 [SMILES: C[C@@H](OCC12CCC(CC1)OC2)[C@H](NC(=O)[C@@H]1CN(C(=O)c2cnn(Cc3ccccc3-c3nn[nH]n3)c2)CC12CN(C(=O)C1(C(F)(F)F)CC1)C2)C(=O)N1CCC2(CCNCC2)CC1] |
| I-118 | 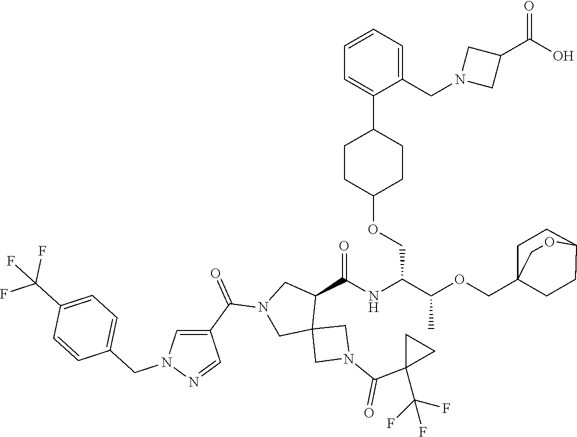 [SMILES: C[C@@H](OCC12CCC(CC1)OC2)[C@@H](COC1CCC(c2ccccc2CN2CC(C(=O)O)C2)CC1)NC(=O)[C@@H]1CN(C(=O)c2cnn(Cc3ccc(C(F)(F)F)cc3)c2)CC12CN(C(=O)C1(C(F)(F)F)CC1)C2] |
| I-119 | 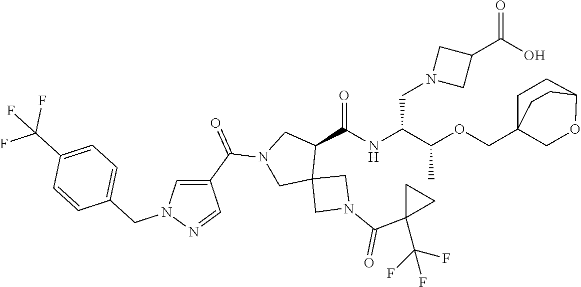 [SMILES: C[C@@H](OCC12CCC(CC1)OC2)[C@@H](CN1CC(C(=O)O)C1)NC(=O)[C@@H]1CN(C(=O)c2cnn(Cc3ccc(C(F)(F)F)cc3)c2)CC12CN(C(=O)C1(C(F)(F)F)CC1)C2] |
| I-120 | 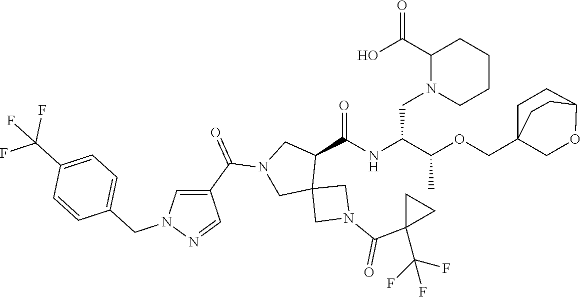 [SMILES: C[C@@H](OCC12CCC(CC1)OC2)[C@@H](CN1CCCCC1C(=O)O)NC(=O)[C@@H]1CN(C(=O)c2cnn(Cc3ccc(C(F)(F)F)cc3)c2)CC12CN(C(=O)C1(C(F)(F)F)CC1)C2] |
| I-121 | 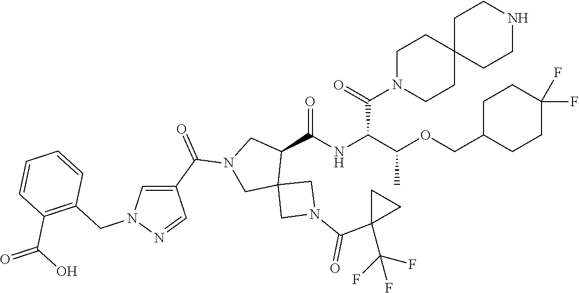 [SMILES: C[C@@H](OCC1CCC(F)(F)CC1)[C@H](NC(=O)[C@@H]1CN(C(=O)c2cnn(Cc3ccccc3C(=O)O)c2)CC12CN(C(=O)C1(C(F)(F)F)CC1)C2)C(=O)N1CCC2(CCNCC2)CC1] |
| I-122 | 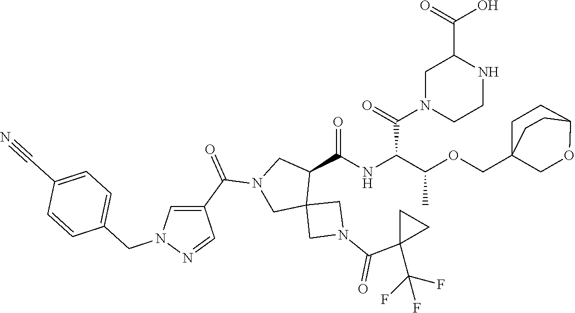 [SMILES: C[C@@H](OCC12CCC(CC1)OC2)[C@H](NC(=O)[C@@H]1CN(C(=O)c2cnn(Cc3ccc(C#N)cc3)c2)CC12CN(C(=O)C1(C(F)(F)F)CC1)C2)C(=O)N1CCNC(C(=O)O)C1] |
| I-123 | 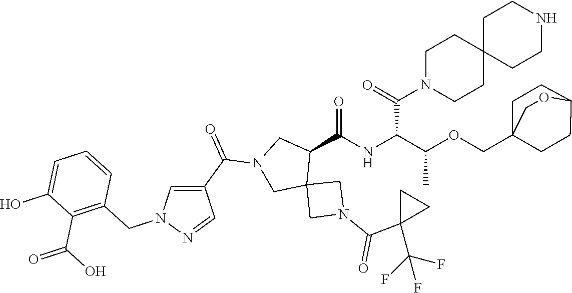 [SMILES: C[C@@H](OCC12CCC(CC1)OC2)[C@H](NC(=O)[C@@H]1CN(C(=O)c2cnn(Cc3cccc(O)c3C(=O)O)c2)CC12CN(C(=O)C1(C(F)(F)F)CC1)C2)C(=O)N1CCC2(CCNCC2)CC1] |
| I-124 | 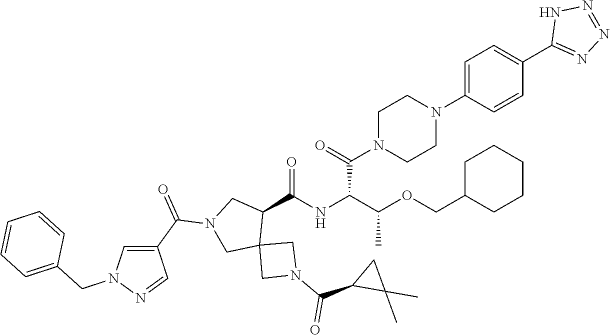 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCN(c2ccc(-c3nnn[nH]3)cc2)CC1] |
| I-125 | 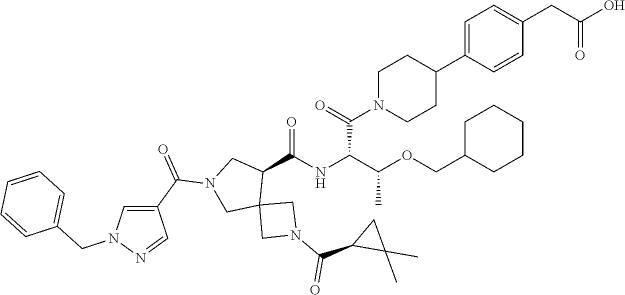 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCC(c2ccc(CC(=O)O)cc2)CC1] |
| I-126 | 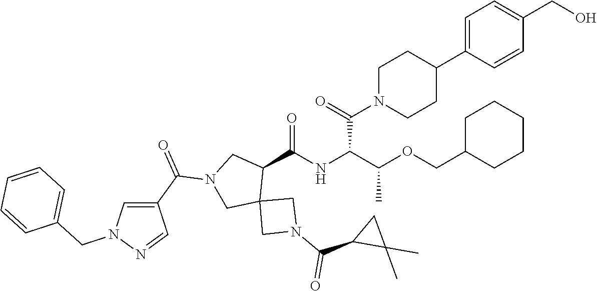 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCC(c2ccc(CO)cc2)CC1] |
| I-127 | 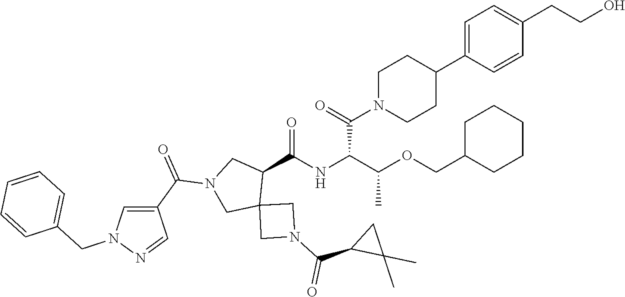 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCC(c2ccc(CCO)cc2)CC1] |
| I-128 | 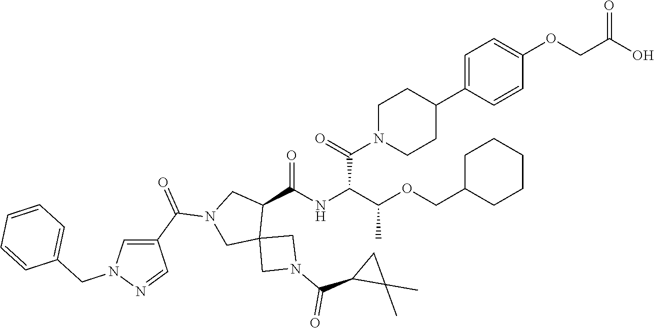 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCC(c2ccc(OCC(=O)O)cc2)CC1] |
| I-129 | 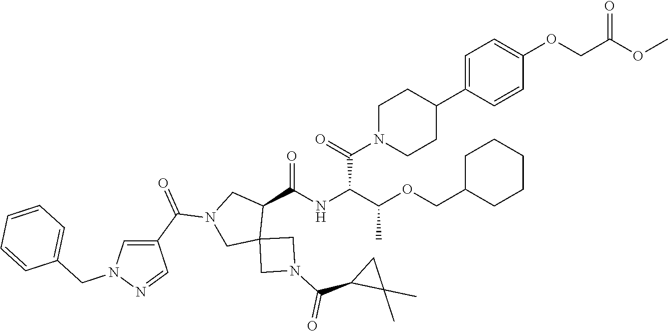 [SMILES: COC(=O)COc1ccc(C2CCN(C(=O)[C@@H](NC(=O)[C@@H]3CN(C(=O)c4cnn(Cc5ccccc5)c4)CC34CN(C(=O)[C@H]3CC3(C)C)C4)[C@@H](C)OCC3CCCCC3)CC2)cc1] |
| I-130 | 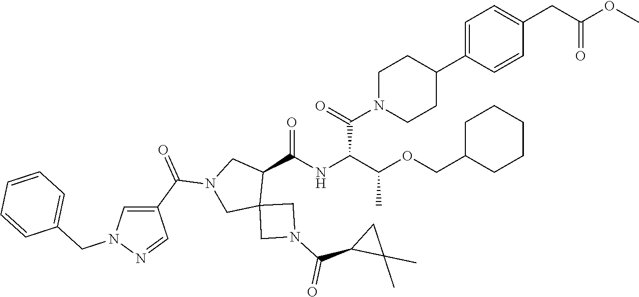 [SMILES: COC(=O)Cc1ccc(C2CCN(C(=O)[C@@H](NC(=O)[C@@H]3CN(C(=O)c4cnn(Cc5ccccc5)c4)CC34CN(C(=O)[C@H]3CC3(C)C)C4)[C@@H](C)OCC3CCCCC3)CC2)cc1] |
| I-131 | 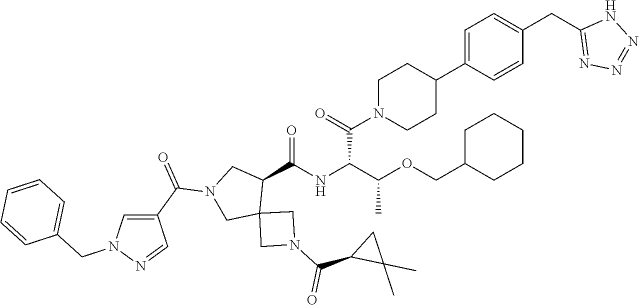 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCC(c2ccc(Cc3nnn[nH]3)cc2)CC1] |
| I-132 | 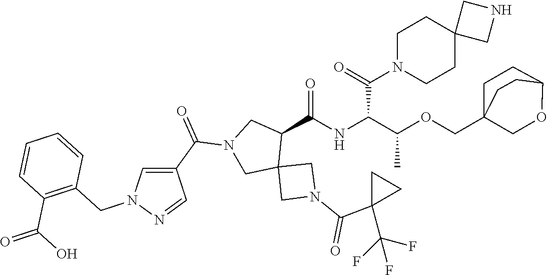 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)C1(C(F)(F)F)CC1)C2)C(=O)N1CCC2(CC1)CNC2] |
| I-133 | 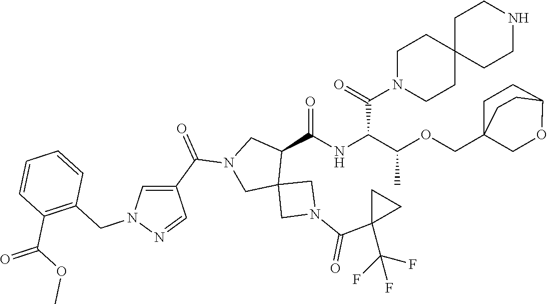 [SMILES: COC(=O)c1ccccc1Cn1cc(C(=O)N2C[C@@H](C(=O)N[C@H](C(=O)N3CCC4(CCNCC4)CC3)[C@@H](C)OCC34CCC(CC3)OC4)C3(C2)CN(C(=O)C2(C(F)(F)F)CC2)C3)cn1] |
| I-134 | 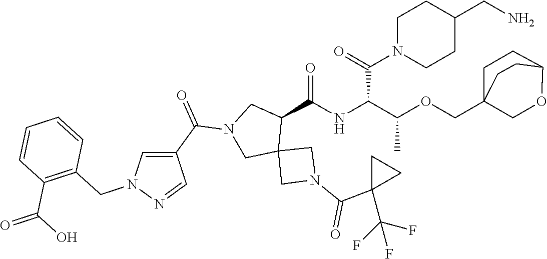 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)C1(C(F)(F)F)CC1)C2)C(=O)N1CCC(CN)CC1] |
| I-135 | 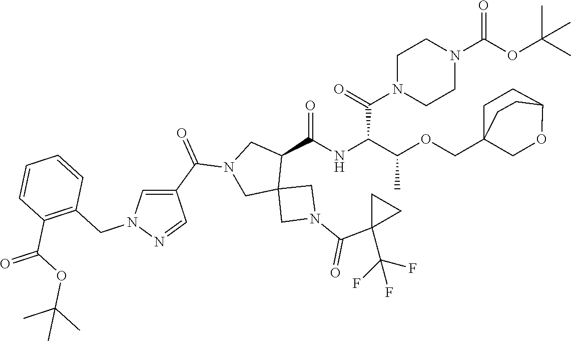 [SMILES: C[C@@H](OCC12CCC(CC1)OC2)[C@H](NC(=O)[C@@H]1CN(C(=O)c2cnn(Cc3ccccc3C(=O)OC(C)(C)C)c2)CC12CN(C(=O)C1(C(F)(F)F)CC1)C2)C(=O)N1CCN(C(=O)OC(C)(C)C)CC1] |
| I-136 | 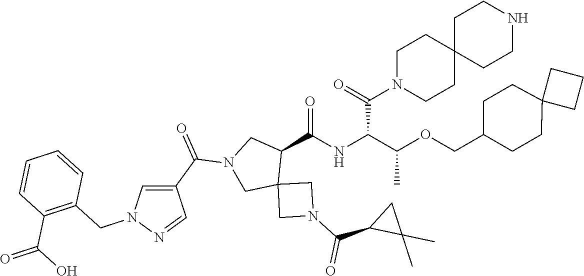 [SMILES: C[C@@H](OCC1CCC2(CCC2)CC1)[C@H](NC(=O)[C@@H]1CN(C(=O)c2cnn(Cc3ccccc3C(=O)O)c2)CC12CN(C(=O)[C@H]1CC1(C)C)C2)C(=O)N1CCC2(CCNCC2)CC1] |
| I-137 | 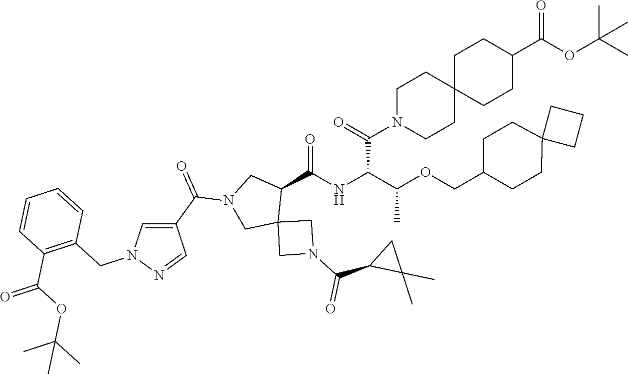 [SMILES: C[C@@H](OCC1CCC2(CCC2)CC1)[C@H](NC(=O)[C@@H]1CN(C(=O)c2cnn(Cc3ccccc3C(=O)OC(C)(C)C)c2)CC12CN(C(=O)[C@H]1CC1(C)C)C2)C(=O)N1CCC2(CCC(C(=O)OC(C)(C)C)CC2)CC1] |

In some embodiments, the present disclosure provides a compound set forth in Table 1, above, or a pharmaceutically acceptable salt thereof. In some embodiments, the disclosure provides a compound set forth in Table 1, above, or a pharmaceutically acceptable salt thereof, and any enantiomers, diastereomers, or conformation isomers thereof. The present disclosure contemplates any and all enantiomers, diastereomers and conformation isomers of a compound shown herein.

In some embodiments, the present disclosure provides a pharmaceutical composition comprising a compound of the present disclosure, or a pharmaceutically acceptable salt thereof, together with a pharmaceutically acceptable carrier, excipient, vehicle, adjuvant or diluent. In some embodiments, the present disclosure provides a pharmaceutical composition comprising a compound set forth in Table 1 above, or a pharmaceutically acceptable salt thereof, together with a pharmaceutically acceptable carrier, excipient, vehicle, adjuvant or diluent. In some embodiments, the pharmaceutical composition further comprises an additional therapeutic agent.

In some embodiments, the present disclosure provides a complex comprising a CDK2 protein and a compound of the present disclosure.

In some embodiments, the present disclosure provides a method of inhibiting the activity of a cyclin-dependent kinase (CDK). In some embodiments, the method comprises contacting a compound of the present disclosure with a CDK. In some embodiments, the compound and the CDK are contacted in vivo. In some embodiments, the compound and the CDK are contacted in vitro. In some embodiments, the CDK is selected from CDK1, CDK2, CDK3, CDK4, CDK5, CDK6, CDK7, CDK8, CDK9, CDK10, CDK11, CDK12 and CDK13. In some embodiments, the CDK is CDK2. In some embodiments, the CDK is CDK3. In some embodiments, the CDK is CDK4. In some embodiments, the CDK is CDK6. In some embodiments, the method inhibits the activity of both CDK2 and CDK3. In some embodiments, the method inhibits the activity of CDK2 and one or both of CDK4 and CDK6.

In some embodiments, the compounds of the present disclosure inhibit the activity of one or more CDKs selected from CDK1, CDK2, CDK3, CDK4, CDK5, CDK6, CDK7, CDK8, CDK9, CDK10, CDK11, CDK12 and CDK13. In some embodiments, the compounds of the present disclosure inhibit CDK2. In some embodiments, the compounds of the present disclosure inhibit CDK3. In some embodiments, the compounds of the present disclosure inhibit CDK4. In some embodiments, the compounds of the present disclosure inhibit CDK5. In some embodiments, the compounds of the present disclosure inhibit CDK6. In some embodiments, the compounds of the present disclosure are CDK2/3 inhibitors. In some embodiments, the compounds of the present disclosure are CDK2/4/6 inhibitors.

In some embodiments, the present disclosure provides compounds that selectively inhibit CDK2 over other cyclin-dependent kinases (CDKs). In some embodiments, the compounds of the present disclosure selectively inhibit CDK2 over one or more other CDKs, selected from CDK1, CDK3, CDK4, CDK5, CDK6, CDK7, CDK8, CDK9, CDK10, CDK11, CDK12 and CDK13. In some embodiments, the compounds of the present disclosure selectively inhibit CDK2 over CDK4. In some embodiments, the compounds of the present disclosure selectively inhibit CDK2 over CDK6. In some embodiments, the compounds of the present disclosure selectively inhibit CDK2 over CDK4 and CDK6.

In some embodiments, the present disclosure provides compounds that selectively inhibit CDK2/cyclin E complexes over other CDK complexes.

### 4. General Methods of Providing the Present Compounds

The compounds of this disclosure may be prepared or isolated in general by synthetic and/or semi-synthetic methods known to those skilled in the art for analogous compounds and by methods described in detail in the Examples, herein.

In the Schemes below, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

Compounds of the present disclosure, including those of Formula I and the compounds of Table 1, can generally be prepared according the methods described below. Reagents and conditions can be modified and substituted using knowledge common to one of ordinary skill in the art, as needed, in order to arrive at the compounds of the present disclosure.

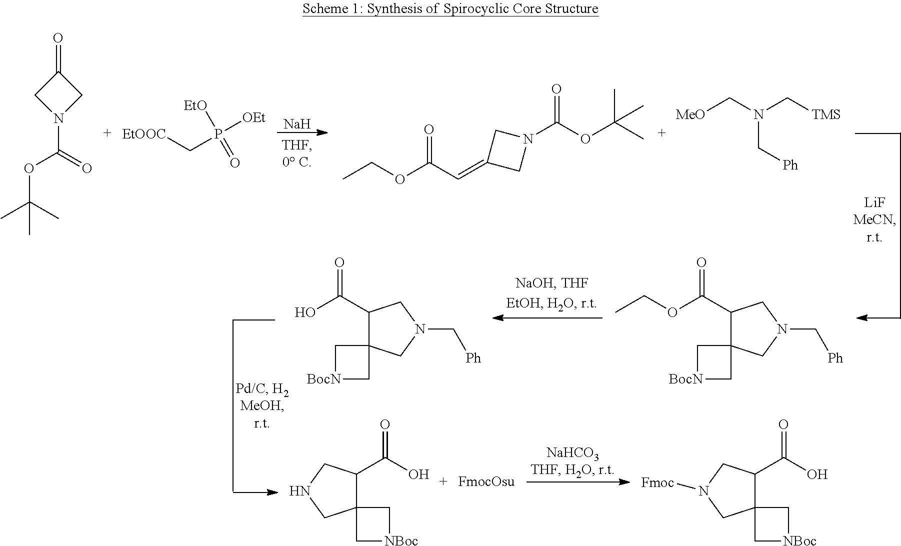

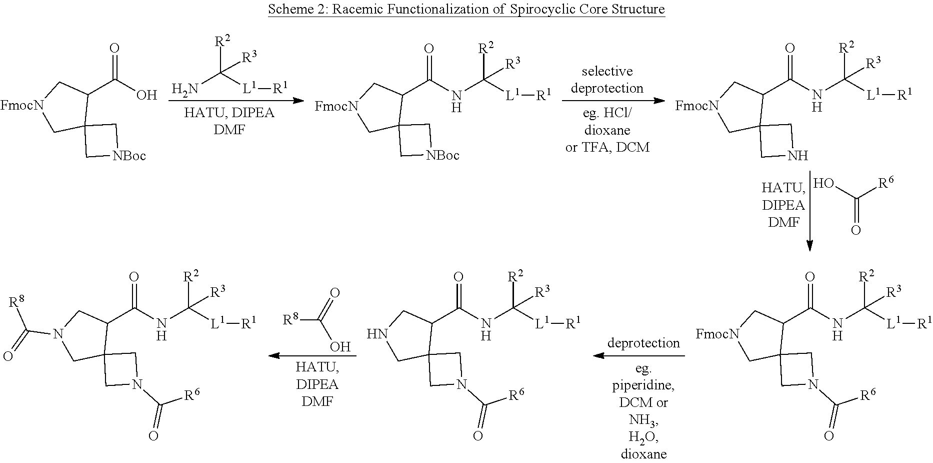

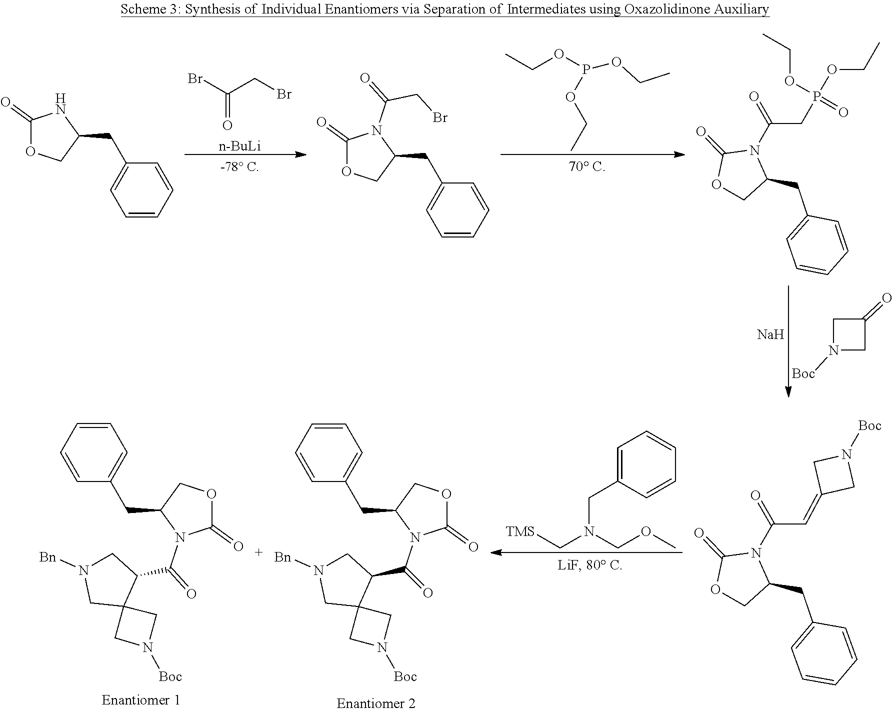

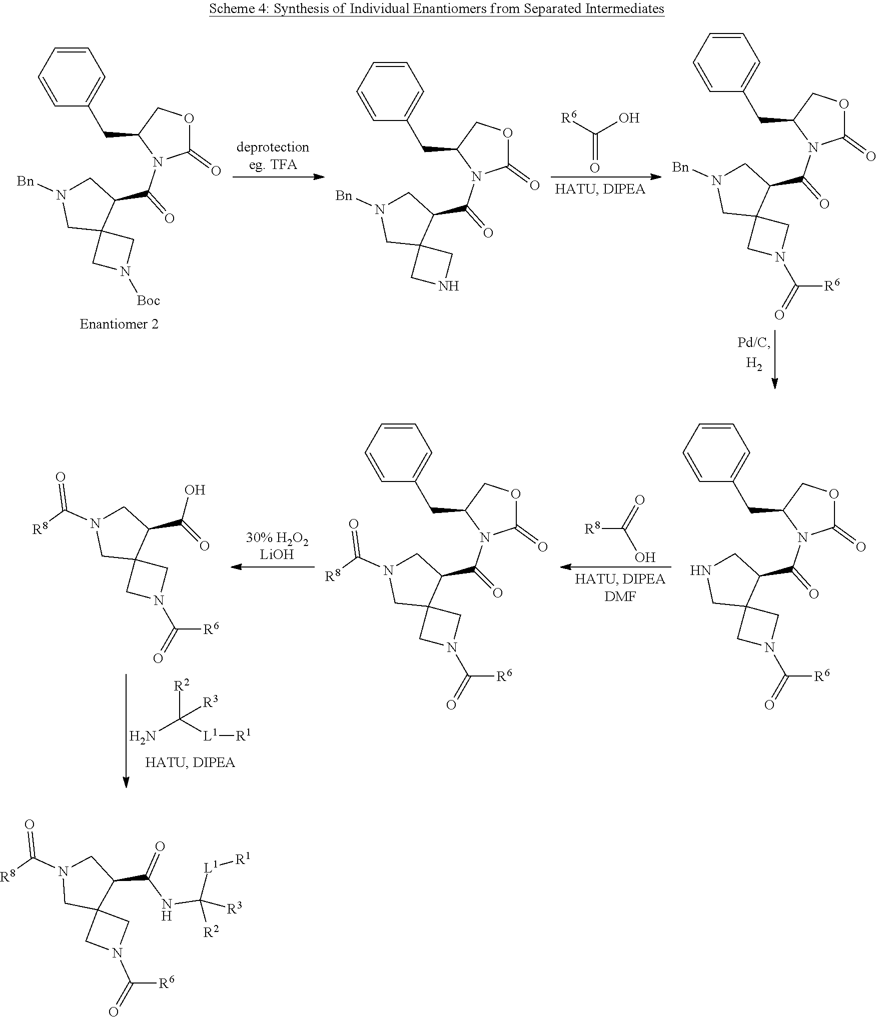

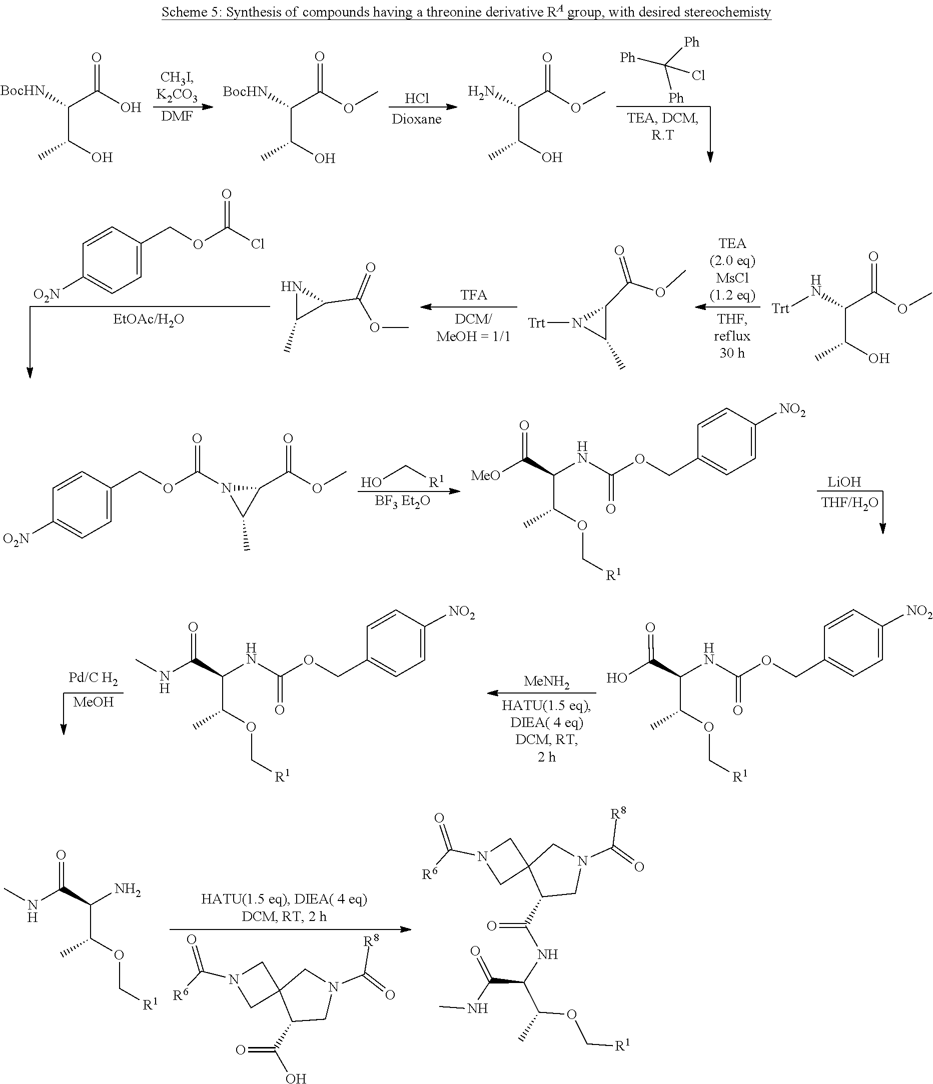

### 5. Uses, Formulation and Administration

### Pharmaceutically Acceptable Compositions

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably inhibit a CDK2 protein, or a mutant thereof, in a biological sample or in a patient. In certain embodiments, the amount of compound in compositions of this disclosure is such that it is effective to measurably inhibit a CDK2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered subcutaneously, orally, intraperitoneally or intravenously. In some embodiments, the compositions are administered orally. In some embodiments, the compositions are administered intraperitoneally. In some embodiments, the compositions are administered intravenously. In some embodiments, the compositions are administered subcutaneously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food.

In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the modulation of the activity CDK2. In some embodiments, the compounds and compositions described herein are CDK2 inhibitors.

In some embodiments, the compounds and compositions of the present disclosure are useful for treating diseases and disorders associated with CDK2 activity, including, but not limited to cancers, myeloproliferative disorders, autoimmune disorders, inflammatory disorders, viral infections, fibrotic disorders, and neurodegenerative disorders.

In some embodiments, the disclosure provides a method of inhibiting the activity of a CDK2, the method comprising contacting a compound of the present disclosure, or a pharmaceutically acceptable salt thereof with the CDK2. In some embodiments, the contacting takes place in vitro. In some embodiments, the contacting takes place in vivo.

In some embodiments, the disclosure provides a method of treating, preventing or lessening the severity of a disease or disorder associated with CDK2 activity in a patient, including, but not limited to cancers, myeloproliferative disorders, autoimmune disorders, inflammatory disorders, fibrotic disorders, and neurodegenerative disorders, said method comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the treatment of a disease or disorder associated with CDK2 activity.

The disclosure further provides a compound of the present disclosure, or a pharmaceutically acceptable salt thereof, or a pharmaceutical composition comprising an effective amount of a compound of the present disclosure, or a pharmaceutically acceptable salt thereof, for use in the manufacture of a medicament for treating a disease or disorder associated with CDK2 activity.

In some embodiments, the disease or disorder associated with CDK2 activity is a CDK2-mediated disease or disorder. In some embodiments, the disease or disorder associated with CDK2 activity is a disease or disorder caused by CDK2 over-activity.

In some embodiments, the disease or disorder associated with CDK2 activity is cancer.

In some embodiments, the cancer is selected from breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

In some embodiments, the cancer is characterized by amplification or overexpression of CCNE1 and/or CCNE2.

In some embodiments, the cancer is breast cancer. In some embodiments, the breast cancer is a breast cancer selected from ER-positive/HR-positive breast cancer, HER2-negative breast cancer, ER-positive/HR-positive breast cancer, HER2-positive breast cancer, triple negative breast cancer (TNBC), inflammatory breast cancer, endocrine resistant breast cancer, trastuzumab resistant breast cancer, breast cancer with primary or acquired resistance to CDK4/CDK6 inhibition, advanced breast cancer and metastatic breast cancer. In some embodiments the breast cancer is characterized by amplification or overexpression of CCNE1 and/or CCNE2.

In some embodiments, the cancer is ovarian cancer. In some embodiments, the ovarian cancer is high-grade serous ovarian cancer (HGSOC). In some embodiments the ovarian cancer is characterized by amplification or overexpression of CCNE1 and/or CCNE2.

In some embodiments, the cancer is bladder cancer. In some embodiments, the bladder cancer is characterized by amplification or overexpression of CCNE1 and/or CCNE2.

In some embodiments, the cancer is uterine cancer. In some embodiments, the uterine cancer is characterized by amplification or overexpression of CCNE1 and/or CCNE2.

In some embodiments, the cancer is prostate cancer. In some embodiments, the prostate cancer is characterized by amplification or overexpression of CCNE1 and/or CCNE2.

In some embodiments, the cancer is lung cancer. In some embodiments, the lung cancer is a lung cancer selected from non-small cell lung cancer, small cell lung cancer, squamous cell carcinoma, adenocarcinoma, and mesothelioma. In some embodiments, the lung cancer is characterized by amplification or overexpression of CCNE1 and/or CCNE2. In some embodiments, the lung cancer is CCNE1 amplified squamous cell carcinoma or CCNE1 amplified adenocarcinoma.

In some embodiments, the cancer is head and neck cancer. In some embodiments, the head and neck cancer is characterized by amplification or overexpression of CCNE1 and/or CCNE2.

In some embodiments, the cancer is colorectal cancer. In some embodiments, the colorectal cancer is characterized by amplification or overexpression of CCNE1 and/or CCNE2.

In some embodiments, the cancer is kidney cancer. In some embodiments, the kidney cancer is renal cell carcinoma (RCC). In some embodiments, the kidney cancer is characterized by amplification or overexpression of CCNE1 and/or CCNE2.

In some embodiments, the cancer is liver cancer. In some embodiments, the liver cancer is hepatocellular carcinoma (HCC). In some embodiments, the liver cancer is characterized by amplification or overexpression of CCNE1 and/or CCNE2.

In some embodiments, the cancer is pancreatic cancer. In some embodiments, the pancreatic cancer is characterized by amplification or overexpression of CCNE1 and/or CCNE2.

In some embodiments, the cancer is stomach cancer. In some embodiments, the stomach cancer is characterized by amplification or overexpression of CCNE1 and/or CCNE2.

In some embodiments, the cancer is melanoma. In some embodiments, the melanoma is characterized by amplification or overexpression of CCNE1 and/or CCNE2. CDK2 expression is regulated by essential melanocytic transcription factor MITF. It has been found that CDK2 depletion suppresses the growth of melanoma (Du et al., Cancer Cell. 2004 December; 6(6): 565-576)

In some embodiments, the cancer is thyroid cancer. In some embodiments, the thyroid cancer is characterized by amplification or overexpression of CCNE1 and/or CCNE2.

In some embodiments, the disease or disorder associated with CDK2 activity is a myeloproliferative disorder.

In some embodiments, the disease or disorder associated with CDK2 activity is a neurodegenerative disease or disorder. In some embodiments, the neurodegenerative disease or disorder is Alzheimer's disease (AD). It has been reported that neuronal cell death in subjects suffering from AD is preceded by cell cycle events. Inhibition of one or more CDKs can inhibit cell cycle events and therefore stave off neuronal cell death (Yang et al., J Neurosci. 2003 Apr. 1; 23(7):2557-2563).

Tn some embodiments, the disease or disorder associated with CDK2 activity is a liver disease.

In some embodiments, the disease or disorder associated with CDK2 activity is liver fibrosis. It has been reported that CCNE1 knockout mice do not develop liver fibrosis upon exposure to pro-fibrotic toxin CCl₄, suggesting that liver fibrosis can be treated via administration of a CDK2 inhibitor (Nevzorova, et al., Hepatology. 2012 September; 56(3): 1140-1149.)

In some embodiments, the disease or disorder associated with CDK2 activity is Cushing disease. Pituitary cyclin E/E2F1 signaling is a molecular mechanism underlying neuroendocrine regulation of the hypothalamic-pituitary-adrenal axis, and therefore provides a subcellular therapeutic target for CDK2 inhibitors of pituitary ACTH-dependent hypercortisolism, also known as Cushing disease (Liu, et al., J Clin Endocrinol Metab. 2015 July; 100(7): 2557-2564).

In some embodiments, the disease or disorder associated with CDK2 activity is a kidney disease.

In some embodiments, the disease or disorder associated with CDK2 activity is polycystic kidney disease. It has been reported that CDK2/CDK5 inhibitor roscovitine yields effective arrest of cystic kidney disease in mouse models of polycystic kidney disease (Bukanov, et al., Nature. 2006 Dec. 14; 444(7121):949-52).

In some embodiments, the disease or disorder associated with CDK2 activity is an autoimmune disorder. CDK2 ablation has been shown to promote immune tolerance by supporting the function of regulatory T cells (Chunder et al., J Immunol. 2012 Dec. 15; 189(12):5659-66).

In some embodiments, the disease or disorder associated with CDK2 activity is an inflammatory disorder. Cyclin E ablation has been shown to attenuate hepatitis in mice, while p27 knockout mice display exacerbation of renal inflammation (Ehedego et al., Oncogene. 2018 June; 37(25):3329-3339; Ophascharoensuk et al., Nat Med. 1998 May; 4(5):575-80). In some embodiments, the inflammatory disorder is hepatitis.

Tn some embodiments, the compounds and compositions of the present disclosure are useful as male contraceptives. Based on the finding that male CDK2 knockout mice are sterile, CDK2 inhibitors have been studied as possible male contraceptives (Faber, et al., Biol Reprod. 2020 August; 103(2): 357-367). In some embodiments, the present disclosure provides a method of reducing male fertility comprising administering to a patient in need thereof, a compound of the present disclosure, or a pharmaceutically acceptable salt thereof, or a pharmaceutical composition comprising an effective amount of a compound of the present disclosure, or a pharmaceutically acceptable salt thereof.

In some embodiments, the compounds and compositions of the present disclosure are useful for treating diseases and disorders associated with CDK5 activity, including, but not limited to cancers, myeloproliferative disorders, autoimmune disorders, inflammatory disorders, viral infections, fibrotic disorders, and neurodegenerative disorders. In some embodiments, the compounds and compositions of the present disclosure are useful for treating neurodegenerative disorders associated with CDK5 activity.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents that the compounds of the present disclosure may also be combined with include, without limitation: endocrine therapeutic agents, chemotherapeutic agents and other CDK inhibitory compounds.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of an endocrine therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional CDK inhibitory compounds. In some embodiments, the one or more additional CDK inhibitory compounds are CDK4, or CDK4/CDK6 inhibitors. In some embodiments, the one or more additional CDK inhibitory compounds are CDK4, CDK6, CDK7 or CDK4/CDK6 inhibitors. In some embodiments, the one or more additional CDK inhibitory compounds are CDK4 inhibitors. In some embodiments, the one or more additional CDK inhibitory compounds are CDK6 inhibitors.

In some embodiments, the one or more additional CDK inhibitory compounds are CDK7 inhibitors. In some embodiments, the one or more additional CDK inhibitory compounds are CDK4/CDK6 inhibitors.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of a chemotherapeutic agent. In some embodiments, the chemotherapeutic agent is a taxane. In some embodiments, the chemotherapeutic agent is a platinum agent. In some embodiments, the chemotherapeutic agent is trastuzumab.

As used herein, the term “combination,” “combined,” and related terms refers to the simultaneous or sequential administration of therapeutic agents in accordance with this disclosure.

For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition the present disclosure are administered as a multiple dosage regimen within greater than 24 hours apart.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### EXAMPLES

As depicted in the Examples below, in certain exemplary embodiments, compounds are prepared according to the general procedures provided herein. It will be appreciated that, although the general methods depict the synthesis of certain compounds of the present disclosure, the general methods, and other methods known to one of ordinary skill in the art, can be applied to all compounds and subclasses and species of each of these compounds, as described herein.

### Example 1: Synthesis Procedures

### Synthesis of 5-(1-(O-((2-oxabicyclo[2.2.2]octan-4-yl)methyl)-n-((s)-2-((s)-2,2-dimethylcyclopropane-1-carbonyl)-6-(5-hydroxypyrazine-2-carbonyl)-2,6-diazaspiro[3.4]octane-8-carbonyl)-L-threonyl) piperidin-4-yl)-2-(trifluoromethyl)benzoic acid (I-90)

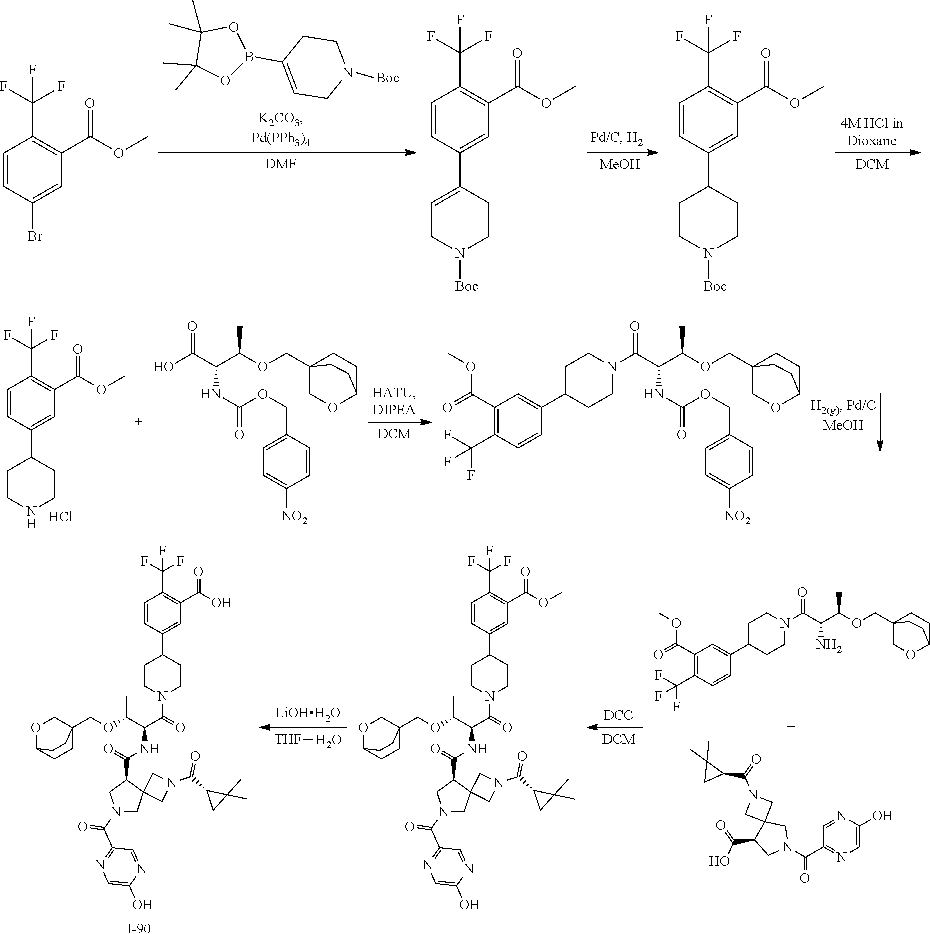

Synthesis of tert-Butyl 4-(3-(methoxycarbonyl)-4-(trifluoromethyl)phenyl)-3,6-dihydropyridine-1 (2H)-carboxylate: To a stirred solution of methyl 5-bromo-2-(trifluoromethyl)benzoate (0.750 g, 2.65 mmol) and tert-butyl 4-(4,4,5,5-tetramethyl-1,3,2-dioxaborolan-2-yl)-3,6-dihydropyridine-1 (2H)-carboxylate (0.819 g, 2.65 mmol) in a DMF (10 mL) was added K₂CO₃ (1.09 g, 7.95 mmol) at room temperature. The reaction mixture was degassed (purging with nitrogen) for 20 min followed by the addition of Pd(PPh₃)₄ (0.153 g, 0.132 mmol) at the same temperature and heated the reaction mixture at 90° C. for 3 h. Reaction mixture was cooled to room temperature, diluted with water (250 mL) and was extracted with diethyl ether (200 mL×3). The combined organic extracts were dried over anhydrous Na₂SO₄ and concentrated under reduce pressure. Obtained crude was combined with an identically prepared one more batch and the combined crude product was purified by silica gel column chromatography, using ethyl acetate-hexane=0:10→1:9 as gradient, to afforded tert-butyl 4-(3-(methoxycarbonyl)-4-(trifluoromethyl)phenyl)-3,6-dihydropyridine-1 (2H)-carboxylate (1.5 g, 73%) as a colourless liquid. MS: [MH]⁺286.3.

Synthesis of tert-Butyl 4-(3-(methoxycarbonyl)-4-(trifluoromethyl)phenyl)piperidine-1-carboxylate: 10% Pd on activated carbon (0.500 g) was added carefully to a stirred solution of tert-butyl 4-(3-(methoxycarbonyl)-4-(trifluoromethyl)phenyl)-3,6-dihydropyridine-1 (2H)-carboxylate (1.00 g, 2.59 mmol) in methanol (12.0 mL) at room temperature under nitrogen and the resulting mixture was hydrogenated under balloon pressure at the same temperature for 2 h. [Another batch (0.500 g) was performed in parallel and mixed together prior to work-up and purification]. Combined mixtures were filtered through a celite bed, washed the bed with methanol (50 mL) and collected filtrates were concentrated under reduced pressure to afforded tert-butyl 4-(3-(methoxycarbonyl)-4-(trifluoromethyl)phenyl)piperidine-1-carboxylate (1.35 g, 89%; crude) as a colourless oil. MS: [(M-56)+1]⁺ 332.2.

Synthesis of Methyl 5-(piperidin-4-yl)-2-(trifluoromethyl)benzoate: To a stirred solution of tert-butyl 4-(3-(methoxycarbonyl)-4-(trifluoromethyl)phenyl)piperidine-1-carboxylate (0.900 g, 2.32 mmol) in DCM (10 mL) was added 4M HCl in dioxane (10 mL) at 0° C. and the resulting reaction mixture was stirred at room temperature for 1 h. [Another batch (0.500 g) was performed in parallel and mixed together prior to work-up and purification]. Combined mixtures were concentrated under reduced pressure and the obtained crude product was purified by trituration with n-pentane, to afforded methyl 5-(piperidin-4-yl)-2-(trifluoromethyl)benzoate hydrochloride (1.20 g, quantitative; crude) as a white solid. MS: [MH]⁺ 288.3.

Synthesis of Methyl 5-(1-(o-((2-oxabicyclo[2.2.2]octan-4-yl)methyl)-N-(((4-nitrobenzyl)oxy)carbonyl)-L-threonyl)piperidin-4-yl)-2-(trifluoromethyl)benzoate: To a stirred solution of o-((2-oxabicyclo[2.2.2]octan-4-yl)methyl)-N-(((4-nitrobenzyl)oxy)carbonyl)-L-threonine (0.400 g, 0.94 mmol) in DCM (7 mL) were added DIPEA (0.360 g, 2.84 mmol) and HATU (0.720 g, 1.895 mmol) at 0° C. under nitrogen. After 10 min of stirring at the same temperature, was added methyl 5-(piperidin-4-yl)-2-(trifluoromethyl)benzoate (0.408 g, 1.42 mmol) and stirring was continued for 1 h at room temperature. Reaction mixture slowly poured into ice water (50.0 mL) and was extracted with ethyl acetate (50 mL×3). Combined organic extracts were washed with brine (20.0 mL), dried over anhydrous Na₂SO₄ and concentrated under reduced pressure to get a crude product. The crude product was purified by column chromatography on silica gel, using MeOH:DCM=1:19 to afforded methyl 5-(1-(o-((2-oxabicyclo[2.2.2]octan-4-yl)methyl)-N-(((4-nitrobenzyl)oxy)carbonyl)-L-threonyl) piperidin-4-yl)-2-(trifluoromethyl)benzoate (0.800 g, quantitative) as an oil. MS: [MH]⁺ 692.4.

Synthesis of Methyl 5-(1-(o-((2-oxabicyclo[2.2.2]octan-4-yl)methyl)-L-threonyl)piperidin-4-yl)-2-(trifluoromethyl)benzoate: 10% Pd in activated carbon (0.400 g) was added carefully to a stirred solution of methyl 5-(1-(o-((2-oxabicyclo[2.2.2]octan-4-yl)methyl)-N-(((4-nitrobenzyl)oxy)carbonyl)-L-threonyl)piperidin-4-yl)-2-(trifluoromethyl)benzoate (0.800 g, 1.15 mmol) in methanol (10 mL) at room temperature under nitrogen and the resulting mixture was hydrogenated under balloon pressure at the same temperature for 2 h. Reaction mixture was filtered through a celite bed, washed the bed with methanol (50 mL) and collected filtrates were concentrated under reduced pressure. The crude product was purified by column chromatography on silica gel, using MeOH:DCM=1:19 to afforded methyl 5-(1-(o-((2-oxabicyclo[2.2.2]octan-4-yl)methyl)-L-threonyl)piperidin-4-yl)-2-(trifluoromethyl)benzoate (0.320 g, 54%) as a white solid. MS: [MH]⁺ 513.4.

Synthesis of intermediate ((s)-2-((s)-2,2-dimethylcyclopropane-1-carbonyl)-6-(5-hydroxypyrazine-2-carbonyl)-2,6-diazaspiro[3.4]octane-8-carboxylic acid is mentioned under 2-(1-(O-((2-oxabicyclo[2.2.2]octan-4-yl)methyl)-N—((S)-2-((S)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-5-(trifluoromethyl)benzoic acid (I-10)

Synthesis of Methyl 5-(1-(o-((2-oxabicyclo[2.2.2]octan-4-yl)methyl)-n-((s)-2-((s)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-2-(trifluoromethyl)benzoate: To a stirred solution of methyl 5-(1-(o-((2-oxabicyclo[2.2.2]octan-4-yl)methyl)-L-threonyl)piperidin-4-yl)-2-(trifluoromethyl) benzoate (0.307 g, 0.60 mmol) in DCM (3 mL) were added ((s)-2-((s)-2,2-dimethylcyclopropane-1-carbonyl)-6-(5-hydroxypyrazine-2-carbonyl)-2,6-diazaspiro[3.4]octane-8-carboxylic acid (0.150 g, 0.40 mmol) and DCC (0.165 g, 0.802 mmol) sequentially at 0° C. under nitrogen and the resulting reaction mixture was stirred for 3 h at room temperature. Reaction mixture was slowly poured into ice water (50.0 mL) and was extracted with DCM (50.0 mL×3). Combined organic extracts were washed with brine (20.0 mL), dried over anhydrous Na₂SO₄ and concentrated under reduced pressure to get a crude product. The crude product was purified by column chromatography on silica gel, using MeOH:DCM=0:1→1:19 to afforded methyl 5-(1-(o-((2-oxabicyclo[2.2.2]octan-4-yl)methyl)-n-((s)-2-((s)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-2-(trifluoromethyl)benzoate (0.200 g, 57%) as a white solid. MS: [MH]⁻ 869.7.

Synthesis of 5-(1-(O-((2-oxabicyclo[2.2.2]octan-4-yl)methyl)-n-((s)-2-((s)-2,2-dimethylcyclopropane-1-carbonyl)-6-(5-hydroxypyrazine-2-carbonyl)-2,6-diazaspiro[3.4]octane-8-carbonyl)-L-threonyl) piperidin-4-yl)-2-(trifluoromethyl)benzoic acid I-90: To a stirred solution of methyl 5-(1-(o-((2-oxabicyclo[2.2.2]octan-4-yl)methyl)-n-((s)-2-((s)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-2-(trifluoromethyl)benzoate (0.170 g, 0.19 mmol) in a mixture of THF-water-MeOH (6:1.5:1.5; 4.5 mL) was added lithium hydroxide monohydrate (0.024 g, 0.58 mmol) at room temperature and stirred for 3 h at the same temperature. Reaction was concentrated under reduced pressure, obtained crude was diluted with water (25 mL), acidified (pH ~2-3) with an aqueous solution of 1N HCl and was extracted with ethyl acetate (50 mL×3). Combined organic extracts were washed with brine (20.0 mL), dried over anhydrous Na₂SO₄ and concentrated under reduced pressure. The crude product was purified by prep-HPLC, 0.1% formic acid in water-acetonitrile as gradient, to afford 5-(1-(o-((2-oxabicyclo[2.2.2]octan-4-yl)methyl)-n-((s)-2-((s)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-2-(trifluoromethyl)benzoic acid I-90 (0.040 g, 24%) as an off white solid. ¹H-NMR (400 MHz, DMSO-d₆) δ 13.75-12.55 (br, 2H), 8.35-8.25 (m, 1H), 7.94-7.89 (m, 1H), 7.77-7.68 (m, 1H), 7.61 (s, 1H), 7.53 (brs, 1H), 6.55-6.60 (br, 1H), 4.92 (br. s, 1H), 4.60-4.52 (m, 1H), 4.19-3.95 (m, 4H), 3.90-3.72 (m, 3H), 3.69-3.64 (m, 2H), 3.56 (s, 2H), 3.50-3.30 (m, 4H; merged in moisture from DMSO-d₆), 3.20-3.10 (m, 2H), 3.01-2.99 (m, 2H), 2.50 (2H, merged in DMSO-d₆), 1.92-1.80 (m, 4H), 1.56-1.51 (t, J=8.8 Hz, 4H), 1.42-1.35 (m, 3H), 1.30-1.29 (m, 1H), 1.12-1.03 (m, 8H), 0.85-0.84 (m, 1H), 0.67-0.66 (m, 1H). MS: [MH]⁺ 855.7.

### Synthesis of intermediate Methyl 2-(1-(O-((2-oxabicyclo[2.2.2]octan-4-yl)methyl)-L-threonyl)piperidin-4-yl)-5-(trifluoromethyl)benzoate

### Synthesis of ethyl 2-(piperidin-4-yl)-5-(trifluoromethyl)benzoate

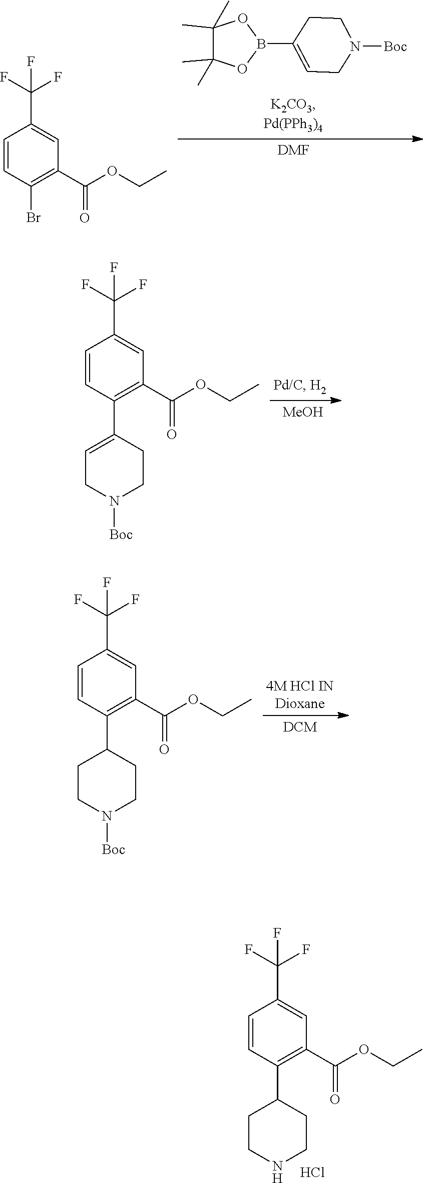

Synthesis of tert-Butyl 4-(2-(ethoxycarbonyl)-4-(trifluoromethyl)phenyl)-3,6-dihydropyridine-1 (2H)-carboxylate: To a stirred solution of ethyl 2-bromo-5-(trifluoromethyl)benzoate (1.0 g, 3.37 mmol) and tert-butyl 4-(4,4,5,5-tetramethyl-1,3,2-dioxaborolan-2-yl)-3,6-dihydropyridine-1 (2H)-carboxylate (1.04 g, 3.37 mmol) in DMF (10 mL) was added K₂CO₃ (1.39 g, 10.13 mmol) at room temperature. Reaction mixture was degassed (purging with nitrogen) for 20 min, followed by the addition of Pd(PPh₃)₄ (0.19 g, 0.168 mmol) at the same temperature and the resulting mixture was heated at 90° C. for 3 h. [Two more identical batches were performed and worked up all together]. After cooling to room temperature, combined reaction mixtures were diluted with water (250 mL) and extracted with ethyl acetate (500 mL×3). Combined organic extracts were dried over anhydrous Na₂SO₄ and concentrated under reduced pressure. The crude product was purified by silica gel column chromatography, using ethyl acetate-hexane=0:1→0.7:9.3 as gradient, to afforded tert-butyl 4-(2-(ethoxycarbonyl)-4-(trifluoromethyl)phenyl)-3,6-dihydropyridine-1 (2H)-carboxylate (3.0 g, 52%) as a colourless oil. MS: [(M-100)+H]⁺300.3.

Synthesis of tert-Butyl 4-(2-(ethoxycarbonyl)-4-(trifluoromethyl)phenyl)piperidine-1-carboxylate: To a stirred solution of tert-butyl 4-(2-(ethoxycarbonyl)-4-(trifluoromethyl)phenyl)-3,6-dihydropyridine-1 (2H)-carboxylate (2.0 g, 5.01 mmol) in a methanol (20 mL) was added 10% Pd in activated carbon (2.0 g) carefully at room temperature under nitrogen and the resulting mixture was hydrogenated under balloon pressure at the same temperature for 3 h. Reaction mixture was filter through a celite bed, washed the bed with methanol (150 mL) and collected filtrates were concentrated under reduced pressure to afforded tert-butyl 4-(2-(ethoxycarbonyl)-4-(trifluoromethyl)phenyl)piperidine-1-carboxylate (1.8 g, 89%; crude) as a colourless oil. MS: [(M-100)+H]⁺ 302.4.

Synthesis of Ethyl 2-(piperidin-4-yl)-5-(trifluoromethyl)benzoate: To a stirred solution of tert-butyl 4-(2-(ethoxycarbonyl)-4-(trifluoromethyl)phenyl)piperidine-1-carboxylate (1.8 g, 4.48 mmol) in DCM (10 mL) was added 4M HCl in dioxane (15 mL) at 0° C. and stirred at room temperature for 1 h. Reaction mixture was concentrated under reduced pressure to obtained crude product, which was purified by trituration using n-pentane, to afforded ethyl 2-(piperidin-4-yl)-5-(trifluoromethyl)benzoate hydrochloride (1.5 g, quantitative; crude) as a white solid. ¹H-NMR (400 MHz, DMSO-d₆) δ 9.01 (s, 2H), 8.00-7.97 (d, J=11.2 Hz, 2H), 7.65-7.63 (d, J=8.4 Hz, 1H), 4.38-4.33 (m, 2H), 3.57-3.51 (t, J=11.2 Hz, 1H), 3.00 (br. s, 2H), 2.01-1.88 (m, 4H), 1.36-1.33 (t, J=7.2 Hz, 3H). MS: [MH]⁺302.1.

### Synthesis of methyl 2-(1-(O-((2-oxabicyclo[2.2.2]octan-4-yl)methyl)-L-threonyl)piperidin-4-yl)-5-(trifluoromethyl)benzoate

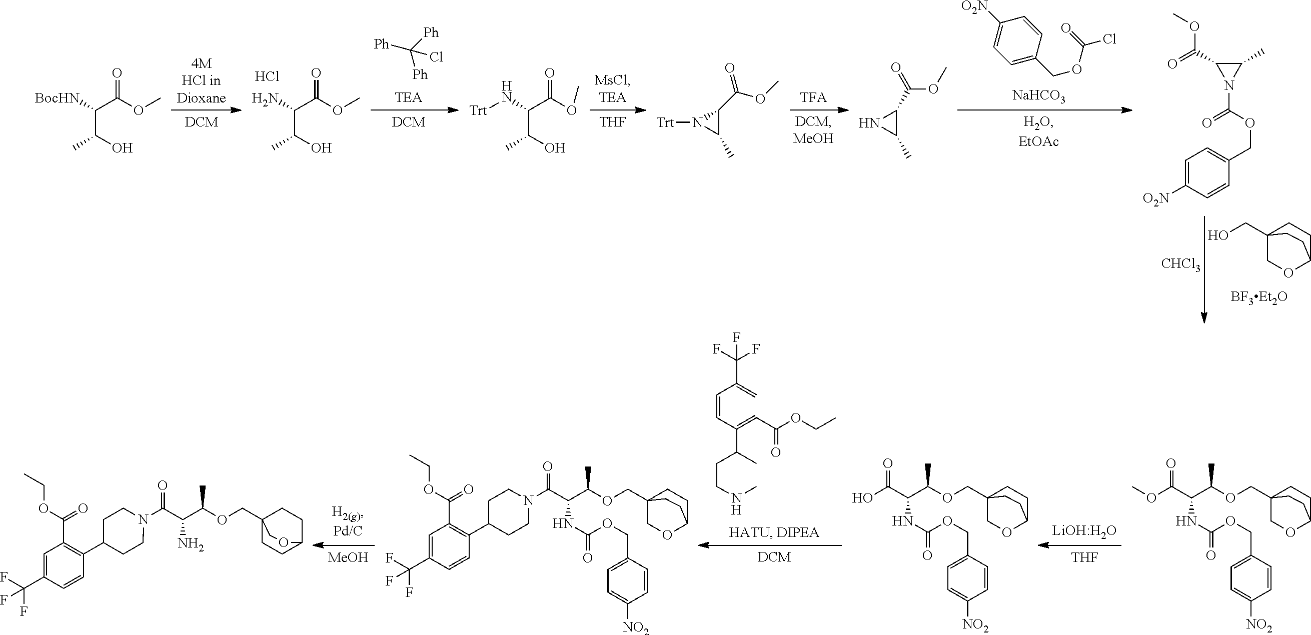

Synthesis of Methyl L-threoninate: To a stirred solution of methyl (tert-butoxycarbonyl)-L-threoninate (20.0 g, 85.78 mmol) in DCM (100 mL) was added 4M HCl in dioxane (40 mL) at 0° C. and stirred at room temperature for 2 h. [Another identical batch was performed and worked up together]. Combined reaction mixtures were concentrated under reduced pressure. The obtained crude product was purified by trituration using n-pentane, to afforded methyl L-threoninate (45.0 g, quantitative; crude). MS: [MH]⁺ 134.2.

Synthesis of Methyl trityl-L-threoninate: To a stirred solution methyl L-threoninate (14.5 g, 109.02 mmol) in DCM (100 mL) were added TEA (55.13 g, 544.82 mmol) and (chloromethanetriyl)tribenzene (36.36 g, 130.75 mmol) sequentially at 0° C. under nitrogen and stirred for 30 min at the same temperature. [Two identical batches with 5.0 g and 12.5 g were performed and combined prior to work-up]. Combined reaction mixtures were slowly poured into water (300 mL) and was extracted with DCM (300 mL×3). Combined organic extracts were wash with brine (200 mL), dried over anhydrous Na₂SO₄ and concentrated in vacuuo. The crude product was purified by silica gel column chromatography, using ethyl acetate-hexane=0:1→2:3 as gradient, to afforded methyl trityl-L-threoninate (48.0 g, 53%) as a yellow sticky compound. ¹H-NMR (400 MHz, DMSO-d₆) δ 7.43-7.42 (d, J=7.6 Hz, 6H), 7.29-7.26 (t, J=7.2 Hz, 6H), 7.21-7.17 (t, J=7.2 Hz, 3H), 5.04-5.03 (d, J=4.4 Hz, 1H), 3.96-3.91 (m, 1H), 3.22-3.16 (m, 1H), 3.02 (s, 3H), 2.69-2.67 (d, J=10.0 Hz, 1H), 1.99 (s, 3H), 1.09-1.07 (d, J=6.4 Hz, 3H).

Synthesis of Methyl (2S, 3S)-3-methyl-1-tritylaziridine-2-carboxylate: To a stirred solution methyl trityl-L-threoninate (35.0 g, 93.28 mmol) in THE (300 mL) was added TEA (26.7 mL, 186.57 mmol) at 0° C. and stirred the reaction mixture for 20 minutes. Mesyl chloride (8.9 mL, 111.94 mmol) was, then, added drop wise into the reaction mixture at the same temperature over the period of 10 minutes and the resulting mixture was stirred at 80° C. for 24 h. Reaction mixture was cool to room temperature and diluted with water (50 mL) and was extracted with ethyl acetate (50 mL×3). Combined organic extracts were washed with brine (50 mL), dried over anhydrous Na₂SO₄ and concentrated in vacuuo. The crude product was purified by silica gel column chromatography, using ethyl acetate-hexane=0:1→0.8 as gradient, to afforded methyl (2S, 3S)-3-methyl-1-tritylaziridine-2-carboxylate (22.0 g, 66%) as a white solid. ¹H-NMR (400 MHz, DMSO-d₆) δ 7.44-7.42 (d, J=7.6 Hz, 6H), 7.34-7.30 (t, J=7.2 Hz, 6H), 7.28-7.19 (m, 3H), 3.67 (s, 3H), 1.73-1.71 (d, J=6.4 Hz, 1H), 1.59-1.53 (m, 1H), 1.29-1.28 (d, J=5.2 Hz, 3H).

Synthesis of 2-Methyl 1-(4-nitrobenzyl) (2S,3S)-3-methylaziridine-1,2-dicarboxylate: To a stirred suspension of methyl (2S, 3S)-3-methyl-1-tritylaziridine-2-carboxylate (2.0 g, 5.60 mmol) in DCM-MeOH mixture (1:3; 20 mL) was added TFA (10.0 mL) at 0° C. and the resulting reaction mixture was stirred at room temperature for 2 h. Reaction mixture was concentrated under reduced pressure till all methanol was distilled-off. Obtained crude was dilute with water (30 mL) and was extracted with diethyl ether (30 mL×3). Collected aqueous layers was basified (pH~9) with the addition of solid NaHCO₃ and diluted with ethyl acetate (30 mL). To the stirred biphasic solution, was added 4-nitrobenzylchloroformate (1.80 g, 8.403 mmol) slowly at 0° C. and stirred the reaction mixture at room temperature for 16 h. Reaction mixture diluted with water (30 mL) and was extracted with ethyl acetate (50 mL×2). Combined organic extracts were washed with brine (30 mL), dried over anhydrous Na₂SO₄ and concentrated in vacuuo. The crude product was purified by silica gel column chromatography, using ethyl acetate-hexane=0:1→3:7 as gradient, to afforded 2-methyl 1-(4-nitrobenzyl) (2S,3S)-3-methylaziridine-1,2-dicarboxylate (1.0 g, 62%) as a white solid. ¹H-NMR (400 MHz, DMSO-d₆) δ 8.25-8.23 (d, J=8.0 Hz, 2H), 7.65-7.63 (d, J=8.0 Hz, 2H), 5.24 (s, 2H), 3.73-3.69 (d, J=14.8 Hz, 3H), 3.43-3.41 (d, J=6.8 Hz, 1H), 3.05-3.01 (m, 1H), 1.21-1.19 (d, J=5.6 Hz, 3H).

Synthesis of Methyl O-((2-oxabicyclo[2.2.2]octan-4-yl)methyl)-N-(((4-nitrobenzyl)oxy)carbonyl)-L-threoninate: To a stirred solution of 2-methyl 1-(4-nitrobenzyl) (2S,3S)-3-methylaziridine-1,2-dicarboxylate (1.0 g, 3.40 mmol) and (2-oxabicyclo[2.2.2]octan-4-yl)methanol (0.480 g, 3.40 mmol) in CHCl₃ (12 mL) was added BF₃·Et₂O (0.240 g, 1.70 mmol) drop wise at 0° C. and the resulting reaction mixture was stirred at room temperature for 2 h. Reaction mixture was concentrated under reduced pressure to obtain a crude product, which was purified by silica gel column chromatography, using ethyl acetate-hexane=0:1→4:6 as gradient, to afforded methyl O-((2-oxabicyclo[2.2.2]octan-4-yl)methyl)-N-(((4-nitrobenzyl)oxy)carbonyl)-L-threoninate (0.550 g, 39%) as a colourless gummy mass. MS: [MH]⁺ 437.4.

Synthesis of O-((2-oxabicyclo[2.2.2]octan-4-yl)methyl)-N-(((4-nitrobenzyl)oxy)carbonyl)-L-threonine: To a stirred solution of methyl O-((2-oxabicyclo[2.2.2]octan-4-yl)methyl)-N-(((4-nitrobenzyl)oxy)carbonyl)-L-threoninate (1.3 g, 2.98 mmol) in a mixture of THF-water (4:1; 10 mL) was added lithium hydroxide monohydrate (0.375 g, 8.94 mmol) at room temperature and the resulting reaction mixture was stirred at room temperature for 3 h. Reaction mixture was concentrated under reduced pressure, obtained crude was diluted with water (5 mL) and was extracted with diethyl ether to get rid of unwanted organic impurities. Separated aqueous layer was acidified (pH ~4) with an aqueous solution of 1N HCl and was extracted with ethyl acetate (50 mL×2). Combined organic extracts were washed with brine (30 mL), dried over anhydrous Na₂SO₄ and concentrated in vacuo to afforded O-((2-oxabicyclo[2.2.2]octan-4-yl)methyl)-N-(((4-nitrobenzyl)oxy)carbonyl)-L-threonine (1.0 g, 82%) as a colourless liquid. MS: [MH]⁺423.4.

Synthesis of Ethyl 2-(1-(O-((2-oxabicyclo[2.2.2]octan-4-yl)methyl)-N-(((4-nitrobenzyl)oxy)carbonyl)-L-threonyl)piperidin-4-yl)-5-(trifluoromethyl)benzoate: To a stirred solution of O-((2-oxabicyclo[2.2.2]octan-4-yl)methyl)-N-(((4-nitrobenzyl)oxy)carbonyl)-L-threonine (0.400 g, 0.94 mmol) in DCM (5.0 mL) were added DIPEA (0.366 g, 2.84 mmol) and HATU (0.722 g, 1.89 mmol) at 0° C. under nitrogen. After 10 minutes of stirring at the same temperature, was added ethyl 2-(piperidin-4-yl)-5-(trifluoromethyl)benzoate (0.427 g, 1.42 mmol) and stirring was continued for another 1 h at room temperature. Reaction mixture was slowly poured into ice water (50 mL) and was extracted with ethyl acetate (100 mL×3). Combined organic extracts were wash with brine (20 mL), dried over anhydrous Na₂SO₄ and concentrated under reduced pressure to get a crude product. The crude product was purified by column chromatography on silica gel (DCM-MeOH=19:1) to afford ethyl 2-(1-(o-((2-oxabicyclo[2.2.2]octan-4-yl)methyl)-N-(((4-nitrobenzyl)oxy)carbonyl)-L-threonyl) piperidin-4-yl)-5-(trifluoromethyl)benzoate (0.350 g, 52%) as an oily liquid. MS: [MH]⁺ 706.5.

Synthesis of Methyl 2-(1-(O-((2-oxabicyclo[2.2.2]octan-4-yl)methyl)-L-threonyl)piperidin-4-yl)-5-(trifluoromethyl)benzoate: To a stirred solution of ethyl 2-(1-(o-((2-oxabicyclo[2.2.2]octan-4-yl)methyl)-N-(((4-nitrobenzyl)oxy)carbonyl)-L-threonyl) piperidin-4-yl)-5-(trifluoromethyl)benzoate (0.350 g, 0.49 mmol) in a methanol (5 mL) was added carefully 10% Pd in activated carbon (0.150 g) at room temperature under nitrogen and the resulting mixture was hydrogenated under balloon pressure at the same temperature for 2 h. Reaction mixture was filtered through a celite bed, washed the bed with methanol (50 mL) and collected filtrates were concentrated under reduced pressure. The crude product was purified by column chromatography on silica gel (DCM:MeOH=19:1) to afford ethyl 2-(1-(o-((2-oxabicyclo[2.2.2]octan-4-yl)methyl)-L-threonyl)piperidin-4-yl)-5-(trifluoromethyl) benzoate (0.190 g, 72%) as a colourless sticky oil. MS: [MH]⁺ 527.5.

### Synthesis of 2-(1-(O-((2-oxabicyclo[2.2.2]octan-4-yl)methyl)-N—((S)-2-((S)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-5-(trifluoromethyl)benzoic acid (I-44)

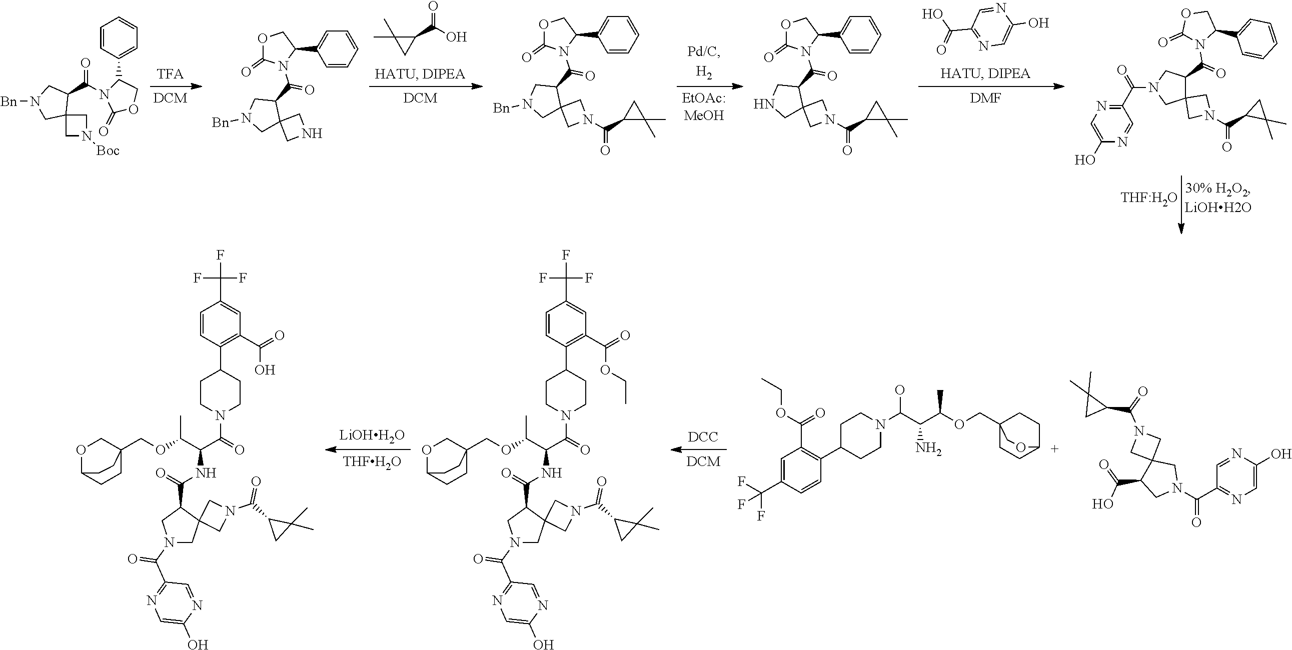

### Synthesis of intermediate (R)-3-((S)-6-benzyl-2,6-diazaspiro[3.4]octane-8-carbonyl)-4-phenyloxazolidin-2-one is mentioned under synthesis for 2-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl) piperidin-4-yl)-5-(trifluoromethyl)benzoic acid I-I

Synthesis of (R)-3-((S)-6-benzyl-2-((S)-2,2-dimethylcyclopropane-1-carbonyl)-2,6-diazaspiro[3.4]octane-8-carbonyl)-4-phenyloxazolidin-2-one: To a stirred solution of (S)-2,2-dimethylcyclopropane-1-carboxylic acid (4.00 g, 35.06 mmol) in DCM (60 mL) were added DIPEA (22.57 g, 174.96 mmol) and HATU (19.95 g, 52.5 mmol) sequentially at 0° C. under nitrogen. After 10 minutes of stirring at the same temperature, was added (R)-3-((S)-6-benzyl-2,6-diazaspiro[3.4]octane-8-carbonyl)-4-phenyloxazolidin-2-one (19.20 g, 49.08 mmol) and stirred at room temperature for 16 h. Reaction mixture was slowly poured into ice water (150 mL) and was extracted with ethyl acetate (150 mL×3). Combined organic extracts were dried over anhydrous Na₂SO₄ and concentrated under reduce pressure. Obtained crude product crude was purified by reverse phase (C-18) silica gel column chromatography using acetonitrile-water=0:1-1:1 as gradient, to afforded (R)-3-((S)-6-benzyl-2-((S)-2,2-dimethylcyclopropane-1-carbonyl)-2,6-diazaspiro[3.4]octane-8-carbonyl)-4-phenyloxazolidin-2-one (8.0 g, 34%) as a brown solid. MS: [MH]⁺ 488.3.

Synthesis of (R)-3-((S)-2-((S)-2,2-dimethylcyclopropane-1-carbonyl)-2,6-diazaspiro[3.4]octane-8-carbonyl)-4-phenyloxazolidin-2-one: To a stirred solution of (R)-3-((S)-6-benzyl-2-((S)-2,2-dimethylcyclopropane-1-carbonyl)-2,6-diazaspiro[3.4]octane-8-carbonyl)-4-phenyloxazolidin-2-one (4.00 g, 8.21 mmol) in a mixture of ethyl acetate-methanol (1:1, 60 mL) was added 10% palladium on activated carbon (2.0 g) at room temperature under nitrogen and the resulting mixture hydrogenated under balloon pressure at room temperature for 9 h. Reaction mixture filtered through celite bed, washed the bed with ethyl acetate (200 mL) and collected filtrates were concentrated under reduced pressure to afford (R)-3-((S)-2-((S)-2,2-dimethylcyclopropane-1-carbonyl)-2,6-diazaspiro[3.4]octane-8-carbonyl)-4-phenyloxazolidin-2-one (3.7 g, quantitative yield; crude) as an off white solid. MS: [MH]⁻ 398.5.

Synthesis of (R)-3-((S)-2-((S)-2,2-dimethylcyclopropane-1-carbonyl)-6-(5-hydroxypyrazine-2-carbonyl)-2,6-diazaspiro[3.4]octane-8-carbonyl)-4-phenyloxazolidin-2-one: To a stirred solution of 5-hydroxypyrazine-2-carboxylic acid (1.8 g, 12.85 mmol) in DCM (60.0 mL) were added DIPEA (4.97 g, 38.56 mmol) and HATU (7.32 g, 19.28 mmol) at 0° C. under nitrogen. After 10 minutes of stirring at the same temperature, was added (R)-3-((S)-2-((S)-2,2-dimethylcyclopropane-1-carbonyl)-2,6-diazaspiro[3.4]octane-8-carbonyl)-4-phenyloxazolidin-2-one (3.57 g, 8.99 mmol; crude) and stirred at room temperature for 12 h. Reaction mixture was slowly poured into ice water (80 mL) and was extracted with ethyl acetate (100 mL×3). Combined organic extracts were washed with brine (150 mL), dried over anhydrous Na₂SO₄ and concentrated under reduced pressure to get a crude product. The resulting crude was purified by reverse phase (C-18) silica gel column chromatography using acetonitrile-water=0:1→4:1 as gradient, to afforded (R)-3-((S)-2-((S)-2,2-dimethylcyclopropane-1-carbonyl)-6-(5-hydroxypyrazine-2-carbonyl)-2,6 diazaspiro[3.4]octane-8-carbonyl)-4-phenyloxazolidin-2-one (1.3 g, 55%) as a brown solid. MS: [MH]⁺ 520.4

Synthesis of (S)-2-((S)-2,2-dimethylcyclopropane-1-carbonyl)-6-(5-hydroxypyrazine-2-carbonyl)-2,6-diazaspiro[3.4]octane-8-carboxylic acid: To a stirred solution of (R)-3-((S)-2-((S)-2,2-dimethylcyclopropane-1-carbonyl)-6-(5-hydroxypyrazine-2-carbonyl)-2,6-diazaspiro[3.4]octane-8-carbonyl)-4-phenyloxazolidin-2-one (1.30 g, 2.50 mmol) in THF (9 mL) was added H₂O₂ (13 mL) at 0° C. and stirred at room temperature for 2 h. An aqueous (1 mL) solution of lithium hydroxide monohydrate (0.168 g, 4.00 mmol) was added at 0° C. and the resulting reaction mixture was stirred at 0° C. for an additional 10 min. Reaction mixture was diluted with water (10 mL) and was extracted with ethyl acetate (20 mL×2) to remove unwanted organic impurities. Aqueous solution was acidified (pH ~2-3) with an aqueous solution of 1N HCl and was extracted with 20% IPA-CHCl₃ solution (100 mL×3). Combined organic extracts were washed with brine (50 mL), dried over anhydrous Na₂SO₄ and concentrated under reduced pressure to get a crude product. The crude product was purified by reverse phase column chromatography using acetonitrile-water=0:1→2:8 to afford (S)-2-((S)-2,2-dimethylcyclopropane-1-carbonyl)-6-(5-hydroxypyrazine-2-carbonyl)-2,6-diazaspiro[3.4]octane-8-carboxylic acid (0.400 g, 42%) as an off white solid. MS: [MH]⁺375.5

Synthesis of Ethyl 2-(1-(O-((2-oxabicyclo[2.2.2]octan-4-yl)methyl)-N—((S)-2-((S)-2,2-dimethylcyclopropane-1-carbonyl)-6-(5-hydroxypyrazine-2-carbonyl)-2,6-diazaspiro[3.4]octane-8-carbonyl)-L-threonyl) piperidin-4-yl)-5-(trifluoromethyl)benzoate: To a stirred solution of (S)-2-((S)-2,2-dimethylcyclopropane-1-carbonyl)-6-(5-hydroxypyrazine-2-carbonyl)-2,6-diazaspiro[3.4]octane-8-carboxylic acid (0.150 g, 0.40 mmol) in DCM (3 mL) were added ethyl 2-(1-(O-((2-oxabicyclo[2.2.2]octan-4-yl)methyl)-L-threonyl)piperidin-4-yl)-5-(trifluoromethyl) benzoate (0.210 g, 0.40 mmol) and DCC (0.165 g, 0.80 mmol) sequentially at 0° C. under nitrogen and the resulting reaction mixture stirred at room temperature for 3 h. Reaction mixture slowly poured into ice water (50 mL) and was extracted with DCM (50 mL×3). Combined organic extracts were wash with brine (20 mL), dried over anhydrous Na₂SO₄ and concentrated under reduced pressure to get a crude product. The crude product was purified by silica gel column chromatography using DCM:MeOH=20→1 as gradient, to afforded Ethyl 2-(1-(O-((2-oxabicyclo[2.2.2]octan-4-yl)methyl)-N—((S)-2-((S)-2,2-dimethylcyclopropane-1-carbonyl)-6-(5-hydroxypyrazine-2-carbonyl)-2,6-diazaspiro[3.4]octane-8-carbonyl)-L-threonyl) piperidin-4-yl)-5-(trifluoromethyl)benzoate (0.180 g, 51%) as a white solid. MS: [MH]⁺ 883.7.

Synthesis of 2-(1-(O-((2-oxabicyclo[2.2.2]octan-4-yl)methyl)-N—((S)-2-((S)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-5-(trifluoromethyl)benzoic acid: To a stirred solution of Ethyl 2-(1-(O-((2-oxabicyclo[2.2.2]octan-4-yl)methyl)-N—((S)-2-((S)-2,2-dimethylcyclopropane-1-carbonyl)-6-(5-hydroxypyrazine-2-carbonyl)-2,6-diazaspiro[3.4]octane-8-carbonyl)-L-threonyl) piperidin-4-yl)-5-(trifluoromethyl)benzoate (0.180 g, 0.203 mmol) in a mixture of THE-water (3:1; 4 mL) was added lithium hydroxide monohydrate (0.0146 g, 0.611 mmol) at room temperature and the resulting reaction mixture was heated at 500° C. for 3 h. After cooling to room temperature, the reaction mass was diluted with water (40 mL) and was extracted with diethyl ether (50 mL) to remove unwanted impurities. Aqueous part was acidified (pH ~2-3) with an aqueous solution of 1N HCl and was extracted with ethyl acetate (50 mL×3). Combined organic extracts were wash with brine (20 mL), dried over anhydrous Na₂SO₄ and concentrated under reduced pressure to get a crude product. The crude product was purified by prep-HPLC, 0.1% formic acid in water-acetonitrile as gradient, to afford 2-(1-(o-((2-oxabicyclo[2.2.2]octan-4-yl)methyl)-N—((S)-2-((S)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-5-(trifluoromethyl) benzoic acid (0.030, 17%) as a white solid. ¹H-NMR (400 MHz, DMSO-d₆) δ 13.48-12.58 (br, 2H), 8.49-8.31 (m, 1H), 7.96-7.76 (m, 3H), 7.59 (brs, 1H), 6.58 (brs, 1H), 5.00-4.82 (m, 1H), 4.62-4.52 (m, 1H), 4.21-3.82 (m, 5H), 3.80-3.50 (m, 6H), 3.50-3.3 (m, 2H; merged in moisture from DMSO-d₆), 3.20-3.01 (m, 2H), 2.70-2.50 (m, 2H; merged in DMSO-d₆), 1.82 (brs, 4H), 1.64-1.48 (m, 4H), 1.45-1.20 (m, 3H), 1.12-1.03 (m, 7H), 0.85 (br. s, 1H), 0.67 (brs, 1H). MS: [MH]⁺ 855.7 (7 aliphatic are there to be accountable and being messy in DMSO-d₆ but it appeared well when added a drop of TFA).

### Synthesis of 5-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-D-threonyl)piperidin-4-yl)-2-(trifluoromethyl)benzoic acid I-82

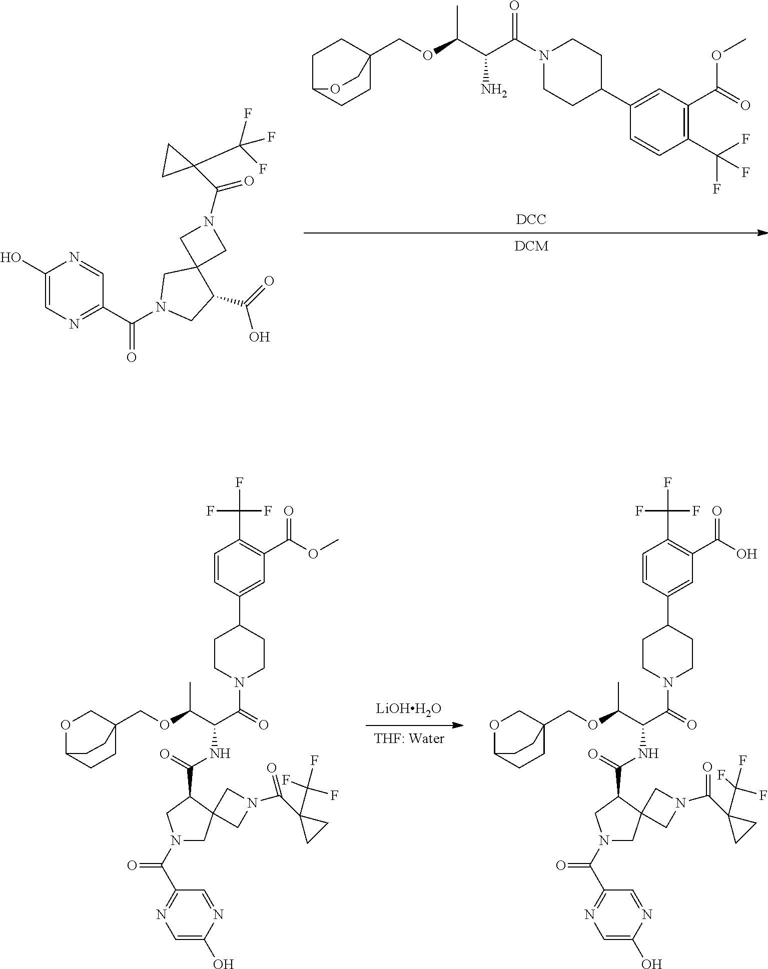

Synthetic procedure of methyl 5-(1-(O-((2-oxabicyclo[2.2.2]octan-4-yl)methyl)-D-threonyl)piperidin-4-yl)-2-(trifluoromethyl)benzoate mentioned under 5-(1-(O-((2-oxabicyclo[2.2.2]octan-4-yl)methyl)-n-((s)-2-((s)-2,2-dimethylcyclopropane-1-carbonyl)-6-(5-hydroxypyrazine-2-carbonyl)-2,6-diazaspiro[3.4]octane-8-carbonyl)-L-threonyl) piperidin-4-yl)-2-(trifluoromethyl)benzoic acid I-90

Synthesis of Methyl 5-(1-(O-((2-oxabicyclo[2.2.2]octan-4-yl)methyl)-N—((S)-6-(5-hydroxypyrazine-2carbonyl)-2-(1-(trifluoromethyl)cyclopropane-1-carbonyl)-2,6-diazaspiro[3.4]octane-8-carbonyl)-D-threonyl) piperidin-4-yl)-2-(trifluoromethyl)benzoate: To a stirred solution of (S)-6-(5-hydroxypyrazine-2-carbonyl)-2-(1-(trifluoromethyl)cyclopropane-1-carbonyl)-2,6-diazaspiro[3.4]octane-8-carboxylic acid (0.200 g, 0.48 mmol) in DCM (3 mL) were added methyl 5-(1-(O-((2-oxabicyclo[2.2.2]octan-4-yl)methyl)-D-threonyl)piperidin-4-yl)-2-(trifluoromethyl)benzoate (0.297 g, 0.57 mmol) and DCC (0.200 g, 0.96 mmol) sequentially at 0° C. under nitrogen and the resulting reaction mixture stirred for 1 h at room temperature. Reaction mixture slowly poured into ice water (40 mL) and extracted with DCM (40 mL×3). Combined organic extracts were wash with brine (20 mL), dried over anhydrous Na₂SO₄ and concentrated under reduced pressure to get a crude product. The crude product was purified by column chromatography on silica gel DCM:MeOH=0:1→1:19 to afforded methyl 5-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl) 2,6diazaspiro[3.4]octane-8-carbonyl)-D-threonyl)piperidin-4-yl)-2-(trifluoromethyl)benzoate (0.160 g, 36%) as an off-white solid. MS: [MH]⁺ 909.6.

Synthesis of 5-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-D-threonyl)piperidin-4-yl)-2-(trifluoromethyl)benzoic acid (I-82): To a stirred solution of methyl 5-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6diazaspiro[3.4]octane-8-carbonyl)-D-threonyl)piperidin-4-yl)-2-(trifluoromethyl)benzoate (0.150 g, 0.16 mmol) in a mixture of THF-water (3:1; 4 mL) was added lithium hydroxide monohydrate (0.020 g, 0.49 mmol) at room temperature and the resulting reaction mixture stirred for 6 h at room temperature. Volatiles were distilled off under reduced pressure, obtained crude was diluted with water (40 mL) and it was extracted with diethyl ether (50 mL×2) to remove unwanted impurities. Aqueous part was acidified (pH ~2-3) with an aqueous solution of 1N HCl and was extracted with ethyl acetate (30 mL×3). Combined organic extracts were wash with brine (20 mL), dried over anhydrous Na₂SO₄ and concentrated under reduced pressure to get a crude product. The crude product was purified by prep-HPLC, 0.1% formic acid in water-acetonitrile as gradient, to afford 5-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1carbonyl)-2,6-diazaspiro[3.4]octane-8-carbonyl)-D-threonyl)piperidin-4-yl)-2-(trifluoromethyl)benzoic acid (0.009 g, 6%) as a white solid. ¹H-NMR (400 MHz, MeOH-d₄) δ 8.30-8.10 (m, 1H), 8.10-7.95 (m, 2H), 7.75-7.60 (m, 2H), 7.59-7.49 (m, 1H), 5.00-4.90 (m, 1H; merged in moisture from MeOH-d₄), 4.71-4.62 (m, 2H), 4.48-4.33 (m, 2H), 4.25-2.19 (m, 3H), 4.15-4.08 (m, 2H), 3.75 (br. s, 4H), 3.50-3.30 (m, 2H, merged in DMSO-d₆), 3.21-2.62 (m, 3H), 2.90-2.77 (m, 1H), 2.12-1.91 (m, 4H), 1.80-1.43 (m, 8H), 1.31-1.15 (m, 8H); MS: [MH]⁺ 895.6.

### Synthesis of 2-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl) piperidin-4-yl)-5-(trifluoromethyl)benzoic acid (I-1)

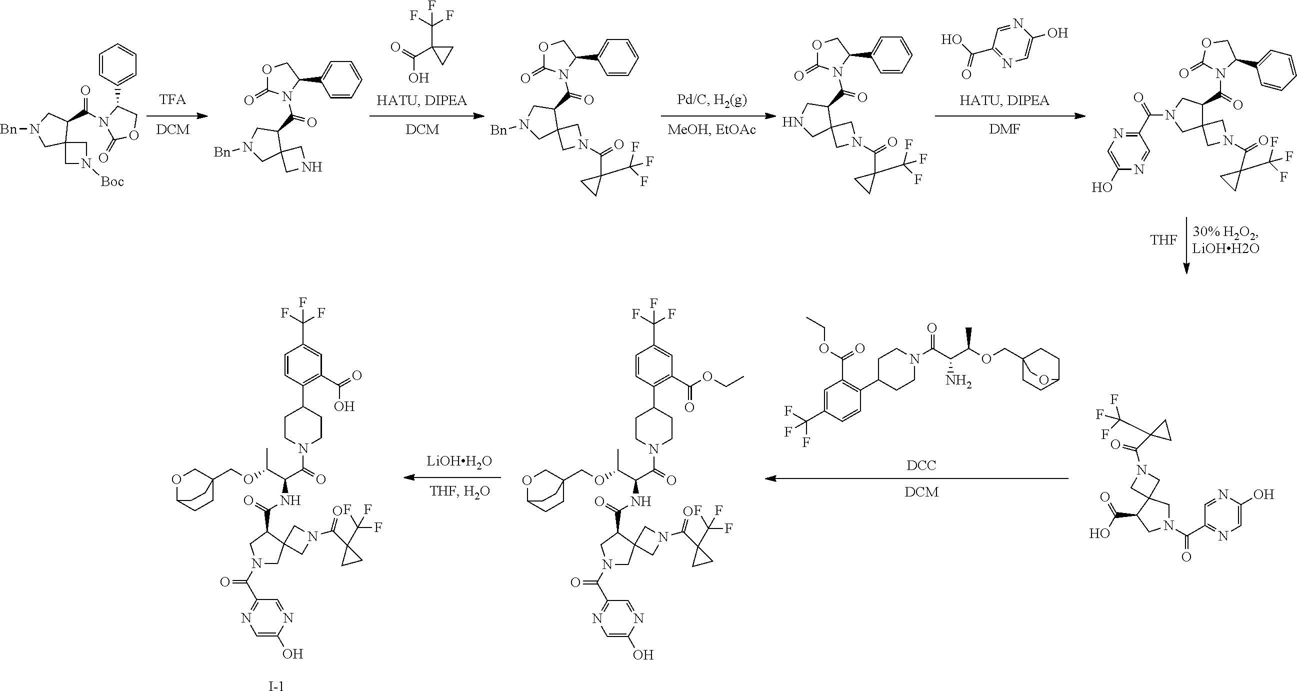

Synthesis of (R)-3-((S)-6-benzyl-2,6-diazaspiro[3.4]octane-8-carbonyl)-4-phenyloxazolidin-2-one: To a stirred solution of tert-butyl (S)-6-benzyl-8-((R)-2-oxo-4-phenyloxazolidine-3-carbonyl)-2,6-diazaspiro[3.4]octane-2-carboxylate (8.0 g, 16.28 mmol) in DCM (40 mL) was added TFA (16 mL) drop-wise at 0° C. under nitrogen and the resulting reaction mixture was stirred at room temperature for 2 h. [An identical batch (8.0 g) was combined with this batch prior to work-up]. Reaction mixture was concentrated under reduced pressure, obtained crude product was diluted with ice water (200 mL), basified (pH~8-9) with an aqueous solution of saturated NaHCO₃ and was extracted with DCM (300 mL×3). Combined organic extracts were dried over anhydrous Na₂SO₄ and concentrated under reduced pressure to afforded (R)-3-((S)-6-benzyl-2,6-diazaspiro[3.4]octane-8-carbonyl)-4-phenyloxazolidin-2-one (11.0 g, quantitative (TFA salt); crude) as an oil, which was used in next step without further purification. MS: [MH]⁻ 392.5.

Synthesis of (R)-3-((S)-6-benzyl-2-(1-(trifluoromethyl)cyclopropane-1-carbonyl)-2,6-diazaspiro[3.4]octane-8-carbonyl)-4-phenyloxazolidin-2-one: To a stirred solution of 1-(trifluoromethyl) cyclopropane-1-carboxylic acid (6.00 g, 38.96 mmol) in DCM (60.0 mL) was added DIPEA (54.2 mL, 311.68 mmol) and HATU (22.20 g, 58.44 mmol) at 0° C. under nitrogen. After 10 minutes of stirring at the same temperature, was added (R)-3-((S)-6-benzyl-2,6-diazaspiro[3.4]octane-8-carbonyl)-4-phenyloxazolidin-2-one (12.18 g, 31.16 mmol) and stirring was continued for 1 h at room temperature. Reaction mixture was slowly poured into ice water (100 mL) and was extracted with ethyl acetate (100 mL×3). Combined organic extracts were wash with brine (100 mL), dried over anhydrous Na₂SO₄ and concentrated under reduced pressure to get a crude product, which was purified by reverse phase (C-18) silica gel column chromatography using acetonitrile-water=0:1→6:1 as gradient, to afford (R)-3-((S)-6-benzyl-2-(1-(trifluoromethyl)cyclopropane-1-carbonyl)-2,6-diazaspiro[3.4]octane-8-carbonyl)-4-phenyloxazolidin-2-one (6.50 g, 80%) as an off white solid. MS: [MH]⁺ 528.5.

Synthesis of (R)-4-phenyl-3-((S)-2-(1-(trifluoromethyl)cyclopropane-1-carbonyl)-2,6-diazaspiro[3.4]octane-8-carbonyl)oxazolidin-2-one: To a stirred solution of (R)-4-phenyl-3-((S)-2-(1-(trifluoromethyl)cyclopropane-1-carbonyl)-2,6-diazaspiro[3.4]octane-8-carbonyl)oxazolidin-2-one (4.00 g, 7.59 mmol) in a mixture of ethyl acetate-methanol (1:1, 20 mL) was added 10% Pd in activated carbon (2.0 g) at room temperature under nitrogen. The resulting reaction mixture hydrogenated under balloon pressure at room temperature for 8 h. [An identical batch (4.0 g) was performed, in parallel and mixed together prior to work-up]. Combined reaction mixtures were filtered through a celite bed, washed the bed with ethyl acetate (200 mL) and collected filtrates were concentrated under reduced pressure to afforded (R)-3-((S)-2-((S)-2,2-dimethylcyclopropane-1-carbonyl)-2,6-diazaspiro[3.4]octane-8-carbonyl)-4-phenyloxazolidin-2-one (7.60 g, quantitative; crude) as an off white solid. MS: [MH]⁺ 438.3.

Synthesis of (R)-3-((S)-6-(5-hydroxypyrazine-2-carbonyl)-2-(1-(trifluoromethyl)cyclopropane-1-carbonyl)-2,6-diazaspiro[3.4]octane-8-carbonyl)-4-phenyloxazolidin-2-one: To a stirred solution 5-hydroxypyrazine-2-carboxylic acid (1.90 g, 13.57 mmol) in DMF were added DIPEA (7.17 mL, 40.71 mmol) and HATU (7.73 g, 20.35 mmol) at 0° C. under nitrogen. After 10 minutes of stirring at the same temperature, was added (R)-3-((S)-2-((S)-2,2-dimethylcyclopropane-1-carbonyl)-2,6-diazaspiro[3.4]octane-8-carbonyl)-4-phenyloxazolidin-2-one (4.10 g, 9.49 mmol) and stirring was continued for 4 h at same temperature. Reaction mixture was slowly poured into ice water (100 mL) and extracted with ethyl acetate (100 mL×3). Combined organic extracts were wash with brine (100 mL), dried over anhydrous Na₂SO₄ and concentrated under reduced pressure. The resulting crude was purified by reverse phase (C-18) silica gel column chromatography using acetonitrile-water=0:1→3:1 as gradient, to afford (R)-3-((S)-6-(5-hydroxypyrazine-2-carbonyl)-2-(1-(trifluoromethyl)cyclopropane-1-carbonyl)-2,6-diazaspiro[3.4]octane-8-carbonyl)-4-phenyloxazolidin-2-one as an off white solid (1.9 g, 25%) as an oil. MS: [MH]⁺ 560.3.

Synthesis of (S)-6-(5-hydroxypyrazine-2-carbonyl)-2-(1-(trifluoromethyl)cyclopropane-1-carbonyl)-2,6-diazaspiro[3.4]octane-8-carboxylic acid: To a stirred solution of (R)-3-((S)-6-(5-hydroxypyrazine-2-carbonyl)-2-(1-(trifluoromethyl)cyclopropane-1-carbonyl)-2,6-diazaspiro[3.4]octane-8-carbonyl)-4-phenyloxazolidin-2-one (0.100 g, 0.17 mmol) in THE (1 mL) was added an aqueous 30% H₂O₂ (1 mL) at 0° C. After stirring at room temperature for 2 h, was added an aqueous (0.2 mL) solution of lithium hydroxide monohydride (0.012 g, 0.286 mmol) into the reaction mixture and the resulting mixture was stirred at same temperature for 1 h. [another batch of 0.500 g was performed and mixed together prior to work-up]. Combined mixtures were diluted with water (20 mL) and was extracted with ethyl acetate (50 mL×2) to remove unwanted organic impurities. The aqueous layer was acidified (pH ~3-4) with formic acid and the resulting solution extracted with 20% IPA in chloroform (50 mL×2). Combined organic extracts were washed with brine (300 mL), dried over anhydrous Na₂SO₄ and concentrated under reduced pressure to get (S)-6-(5-hydroxypyrazine-2-carbonyl)-2-(1-(trifluoromethyl)cyclopropane-1-carbonyl)-2,6-diazaspiro[3.4]octane-8-carboxylic acid (0.300 g, 67%; crude) as a brown solid. MS: [MH]⁺ 415.3.

Synthesis of Ethyl 2-(1-(o-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threnyl)piperidin-4-yl)-5-(trifluoromethyl)benzoate: To a stirred solution of (S)-6-(5-hydroxypyrazine-2-carbonyl)-2-(1-(trifluoromethyl)cyclopropane-1-carbonyl)-2,6-diazaspiro[3.4]octane-8-carboxylic acid (0.115 g, 0.27 mmol) in DCM (2.0 mL) were added ethyl 2-(1-(O-((2-oxabicyclo[2.2.2]octan-4-yl)methyl)-L-threonyl)piperidin-4-yl)-5-(trifluoromethyl) benzoate (0.146 g, 0.27 mmol) and DCC (0.114 g, 0.555 mmol) at 0° C. under nitrogen. The resulting reaction mixture stirred for 2 h at room temperature. Reaction mixture was slowly poured into ice water (50 mL) and was extracted with DCM (50 mL×3). Combined organic extracts were washed with brine (20 mL), dried over anhydrous Na₂SO₄ and concentrated under reduced pressure to get a crude product. The crude product was purified by column chromatography on silica gel (DCM-MeOH=0.8:9.2) to afford methyl ethyl 2-(1-(o-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-5-(trifluoromethyl) benzoate (0.060 g, 23%) as an off white solid. MS: [MH]⁺923.6.

Synthesis of 2-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl) piperidin-4-yl)-5-(trifluoromethyl)benzoic acid: To a stirred solution of ethyl 2-(1-(o-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl) piperidin-4-yl)-5-(trifluoromethyl)benzoate (0.150 g, 0.16 mmol) in a mixture of THF-water (3:1; 2 mL) and methanol (0.3 mL) was added an aqueous (0.2 mL) solution of LiOH·H₂O (0.020 g, 0.48 mmol) at 0° C. and stirred for 3 h at room temperature. Volatiles were distilled off under reduced pressure, obtained crude was acidified (pH ~4) with an aqueous solution of 1N HCl and was extracted with ethyl acetate (50 mL×3). Combined organic extracts washed with brine (20 mL), dried over anhydrous Na₂SO₄ and concentrated under reduced pressure to get a crude product. The crude product was purified by prep-HPLC, 0.1% formic acid in water-acetonitrile as gradient, to afforded 2-(1-(O-((2-oxabicyclo[2.2.2]octan-4-yl)methyl)-N—((S)-6-(5-hydroxypyrazine-2-carbonyl)-2-(1-(trifluoromethyl) cyclopropane-1-carbonyl)-2,6-diazaspiro[3.4]octane-8-carbonyl)-L-threonyl)piperidin-4-yl)-5-(trifluoromethyl) benzoic acid (0.025 g, 17%) as a white solid. ¹H NMR (400 MHz, DMSO-d₆) δ 8.43-8.31 (m, 1H), 8.09-7.89 (m, 2H), 7.80-7.78 (m, 1H), 7.70-7.59 (m, 1H), 7.50-7.41 (m, 1H), 4.92-4.75 (m, 2H), 4.59-4.53 (m, 1H), 4.40-4-30 (m, 1H), 4.30-4.20 (s, 1H), 4.12-4.07 (m, 2H), 3.95-3.90 (m, 3H), 3.87-3.78 (m, 3H), 3.62 (s, 2H), 3.55 (s, 4H), 3.18-3.11 (m, 2H), 3.05-2.98 (m, 1H), 1.81 (br. s, 4H), 1.53 (br. s, 5H), 1.42-1.35 (m, 2H), 1.23-1.14 (m, 4H), 1.10-0.98 (m, 3H). MS: [MH]⁺ 895.7.

### Synthesis of 4-(1-(O-((2-Oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid (I-81)

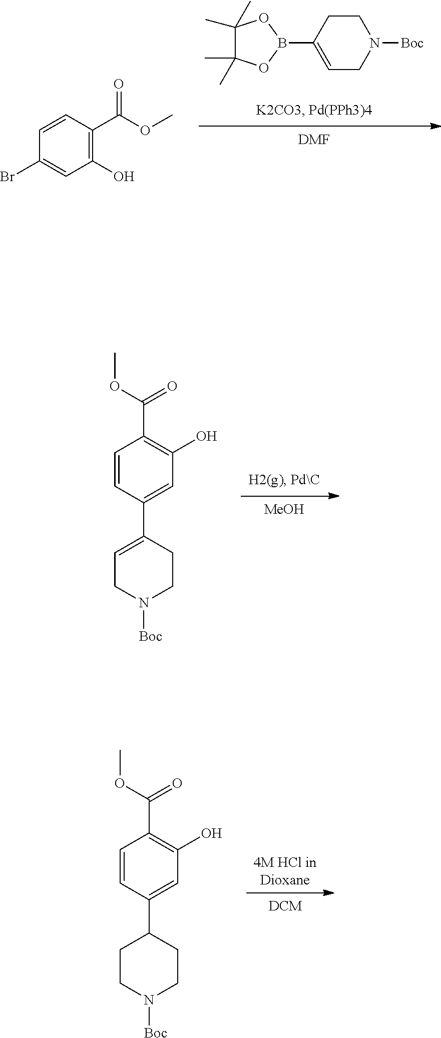

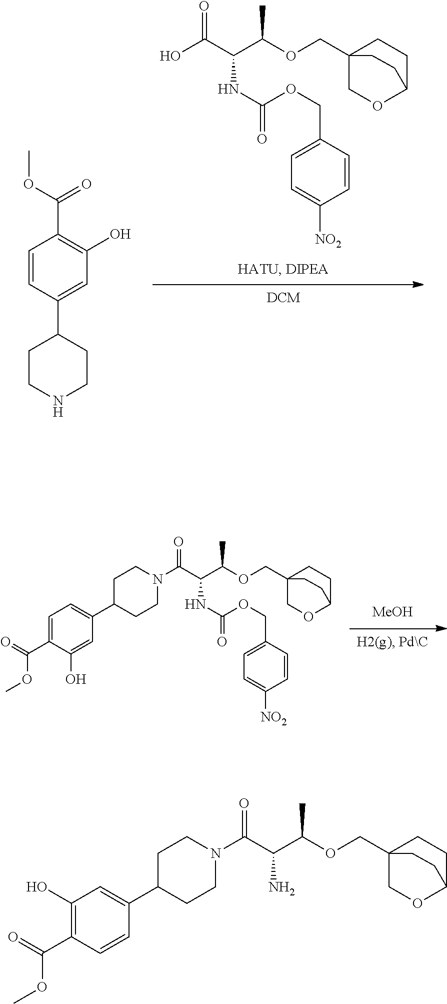

Synthesis of tert-Butyl 4-(3-hydroxy-4-(methoxycarbonyl)phenyl)-3,6-dihydropyridine-1 (2H)-carboxylate: To a stirred solution of methyl 4-bromo-2-hydroxybenzoate (1.0 g, 4.34 mmol) in a DMF (7.0 mL) were added tert-butyl 4-(4,4,5,5-tetramethyl-1,3,2-dioxaborolan-2-yl)-3,6-dihydropyridine-1 (2H)-carboxylate (1.34 g, 4.34 mmol) and K₂CO₃ (1.79 g, 13.04 mmol) at room temperature. The reaction mixture was degassed (purging with nitrogen) for 20 min followed by addition of Pd(PPh₃)₄ (0.250 g, 0.21 mmol) and stirred at 100° C. for 16 h. [Two more batched of 1.0 g each were carried out in parallel and mixed all together prior to work-up]. Combined reaction mixture was cool to room temperature, diluted with water (100 mL) and was extracted with diethyl ether (100 mL×3). Combined organic extracts were washed with brine (200 mL), dried over anhydrous Na₂SO₄ and concentrated under reduce pressure. Obtained crude was purified by silica gel column chromatography, using ethyl acetate-hexane=0:1→0.7:9.3 as gradient, to afford tert-butyl 4-(3-hydroxy-4-(methoxycarbonyl)phenyl)-3,6-dihydropyridine-1 (2H)-carboxylate (1.1 g, 25%) as a colourless liquid. MS: [MH-56]⁺ 278.3.

Synthesis of Tert-butyl 4-(3-hydroxy-4-(methoxycarbonyl)phenyl)piperidine-1-carboxylate: To a stirred solution of tert-butyl 4-(3-hydroxy-4-(methoxycarbonyl)phenyl)-3,6-dihydropyridine-1 (2H)-carboxylate (1.1 g, 3.30 mmol) in a methanol (10 mL) was added 10% Pd in activated carbon (0.550 g) at room temperature under nitrogen and the resulting mixture was hydrogenated under balloon pressure at the same temperature for 2 h. The reaction mixture was filter through a celite bed, washed the bed with methanol (100 mL) and collected filtrates were concentrated under reduced pressure to afforded tert-butyl 4-(3-hydroxy-4-(methoxycarbonyl)phenyl)piperidine-1-carboxylate (0.900 g, 81%; crude) as a colourless oil. MS: [MH-56]⁺ 280.3.

Synthesis of Methyl 2-hydroxy-4-(piperidin-4-yl)benzoate: To a stirred solution of tert-butyl 4-(3-hydroxy-4-(methoxycarbonyl)phenyl)piperidine-1-carboxylate (1.50 g, 4.47 mmol) in DCM (10 mL) was added 4M HCl in dioxane (7 mL) at 0° C. and stirred at room temperature for 1 h. The reaction mixture was concentrated under reduced pressure to obtained crude product which was purified by trituration using n-pentane to afford methyl 2-hydroxy-4-(piperidin-4-yl)benzoate (1.20 g, quantitative; crude) as an off-white solid. MS: [MH]⁺ 236.4.

Synthesis of Methyl 4-(1-(O-((2-oxabicyclo[2.2.2]octan-4-yl)methyl)-N-(((4-nitrobenzyl)oxy)carbonyl)-L-threonyl)piperidin-4-yl)-2-hydroxybenzoate: To a stirred solution of O-((2-oxabicyclo[2.2.2]octan-4-yl)methyl)-N-(((4-nitrobenzyl)oxy)carbonyl)-L-threonine (1.2 g, 2.84 mmol) in DCM (10.0 mL), were added DIPEA (1.1 g, 8.52 mmol) and HATU (2.1 g, 5.68 mmol) at 0° C. under nitrogen. After 10 minutes of stirring at the same temperature, was added methyl 2-hydroxy-4-(piperidin-4-yl)benzoate (1.0 g, 4.26 mmol) and stirring was continued for 1 h at room temperature. The reaction mixture was slowly poured into ice water (70 mL) and was extracted with dichloromethane (100 mL×3), dried over anhydrous Na₂SO₄ and concentrated under reduced pressure to get a crude product. The crude product was purified by silica gel column chromatography, using ethyl acetate-hexane=0:1→6:4 as gradient, to afford methyl 4-(1-(O-((2-oxabicyclo[2.2.2]octan-4-yl)methyl)-N-(((4-nitrobenzyl)oxy)carbonyl)-L-threonyl)piperidin-4-yl)-2-hydroxybenzoate (0.800 g, 44%) as a colorless liquid. MS: [MH]⁺ 640.5.

Synthesis of Methyl 4-(1-(O-((2-oxabicyclo[2.2.2]octan-4-yl)methyl)-L-threonyl)piperidin-4-yl)-2-hydroxybenzoate: To a stirred solution of methyl 4-(1-(O-((2-oxabicyclo[2.2.2]octan-4-yl)methyl)-N-(((4-nitrobenzyl)oxy)carbonyl)-L-threonyl)piperidin-4-yl)-2-hydroxybenzoate (0.750 g, 1.17 mmol) in methanol (5.0 mL) was added 10% Pd in activated carbon (0.350 g) at room temperature under nitrogen and the resulting mixture was hydrogenated under balloon pressure at the same temperature for 1 h. The reaction mixture filtered through celite bed, washed the bed with methanol (50 mL) and collected filtrates were concentrated under reduced pressure. The crude product was purify by silica gel column chromatography, using MeOH-DCM=0:1→1.5:8.5 to afford methyl 4-(1-(O-((2-oxabicyclo[2.2.2]octan-4-yl)methyl)-L-threonyl)piperidin-4-yl)-2-hydroxybenzoate (0.330 g, 61%) as a colour less liquid. MS: [MH]⁺ 461.4

### Synthesis of 1-(4-(Trifluoromethyl)benzyl)-1H-pyrazole-4-carboxylic acid

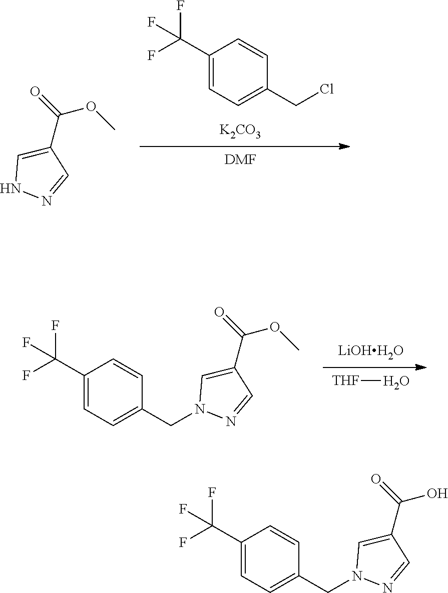

Synthesis of Methyl 1-(4-(trifluoromethyl)benzyl)-1H-pyrazole-4-carboxylate: To a stirred solution of methyl 1H-pyrazole-4-carboxylate (20.0 g, 158.70 mmol) in DMF (200 mL) were added K₂CO₃ (54.70 g, 396.8 mmol) and 1-(chloromethyl)-4-(trifluoromethyl)benzene (36.90 g, 190.41 mmol) sequentially at room temperature and the resulting mixture was stirred at 90° C. for 2 h. Reaction mixture slowly poured into ice water (500 mL), during which a solid was precipitated. Reaction mixture was filtered over a celite bed, obtained residue was washed with cold water and dried under high vacuum to afford methyl 1-(4-(trifluoromethyl)benzyl)-1H-pyrazole-4-carboxylate (30.0 g, 92%; crude) as a white solid. MS: [MH]⁺285.3.

Synthesis of 1-(4-(Trifluoromethyl)benzyl)-1H-pyrazole-4-carboxylic acid: To a stirred solution of methyl 1-(4-(trifluoromethyl)benzyl)-1H-pyrazole-4-carboxylate (15.0 g, 52.81 mmol) in a mixture of THF-water (3:1; 150 mL) was added lithium hydroxide monohydrate (6.65 g, 158.4 mmol) at room temperature and stirred for 3 h at the same temperature. Reaction mixture was concentrated under reduced pressure, obtained crude was diluted with water (100 mL) and was extracted with ethyl acetate (100 mL×2) to remove unwanted impurities. Aqueous part was acidified (pH ~3-4) with 1N HCl aqueous solution and the resulting precipitate collected by filtration. Crude residue was washed with cold water until the pH of the filtrate became neutral (pH ~6-7) and dried under high vacuum to afford 1-(4-(trifluoromethyl)benzyl)-1H-pyrazole-4-carboxylic acid (12.0 g, quant.; crude) as a white solid. MS: [MH]⁺ 271.3.

### Synthesis of 4-(1-(O-((2-Oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid (I-81)

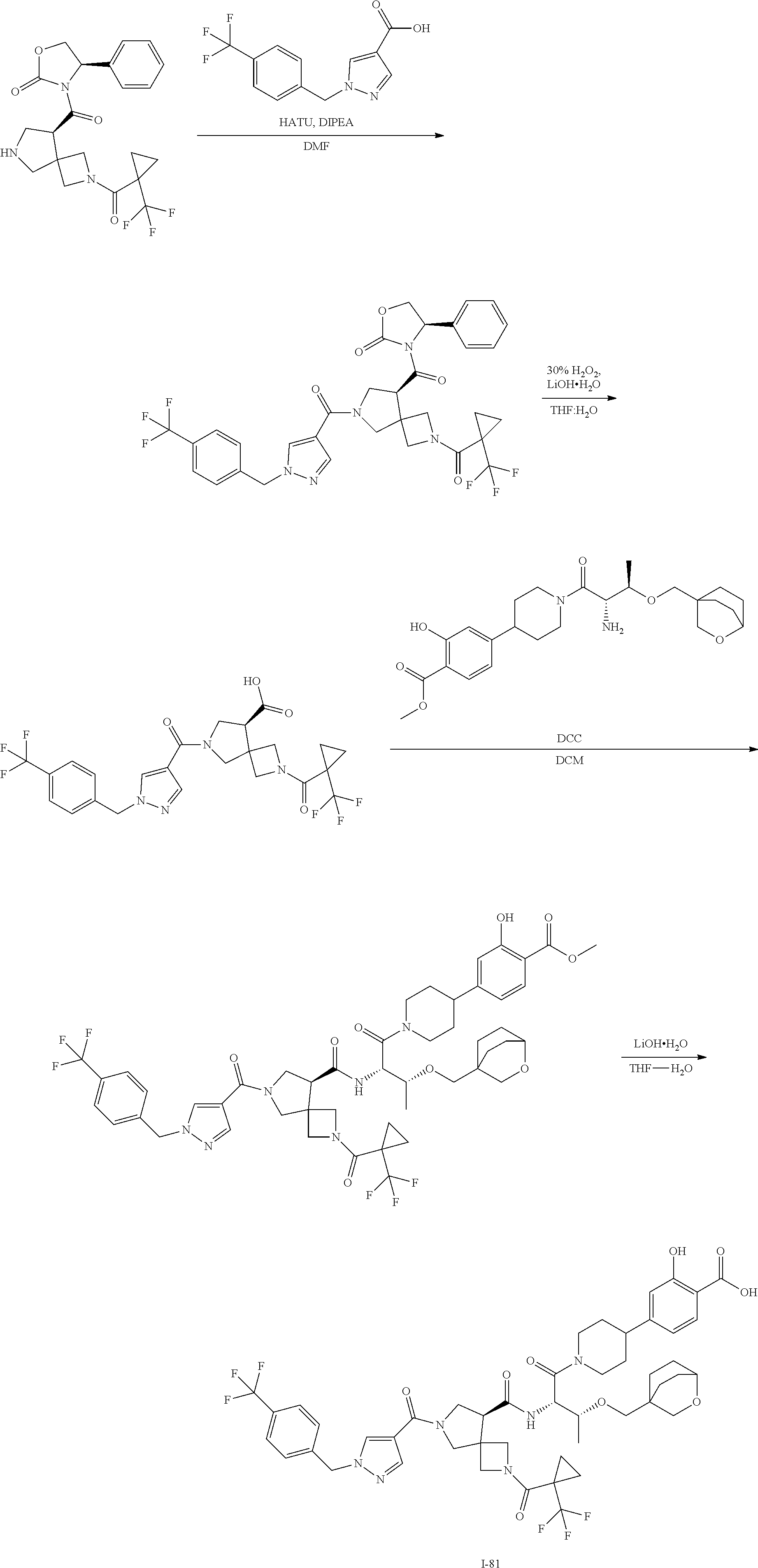

Synthesis of (R)-4-Phenyl-3-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)oxazolidin-2-one: To a stirred solution of 1-(4-(trifluoromethyl)benzyl)-1H-pyrazole-4-carboxylic acid (6.2 g, 22.96 mmol) in DMF (60 mL) were added DIPEA (8.88 g, 68.88 mmol) and HATU (13.08 g, 34.44 mmol) at 0° C. under nitrogen. After 10 minutes of stirring at the same temperature, was added (R)-4-phenyl-3-((S)-2-(1-(trifluoromethyl)cyclopropane-1-carbonyl)-2,6-diazaspiro[3.4]octane-8 carbonyl)oxazolidin-2-one (12.04 g, 27.55 mmol) and stirred at room temperature for another 1 h. The resulting reaction mixture was slowly poured into ice water (150 mL) and was extracted with ethyl acetate (500 mL×2). Combined organic extracts were washed with brine (200 mL) and dried over anhydrous Na₂SO₄, and concentrated under reduced pressure to get a crude product. The crude product was purified by silica gel column chromatography, using MeOH-DCM=0:1→0.5:9.5 as gradient, to afford (R)-4-phenyl-3-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)oxazolidin-2-one (8.0 g, 82%) as a yellow solid. MS: [MH]⁺ 690.3.

Synthesis of (S)-6-(1-(4-(Trifluoromethyl)benzyl)-1H-pyrazole-4-carbonyl)-2-(1 (trifluoromethyl)cyclopropane-1-carbonyl)-2,6-diazaspiro[3.4]octane-8-carboxylic acid: To a stirred solution of (R)-4-phenyl-3-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)oxazolidin-2-one (2.0 g, 2.90 mmol) in a mixture of THF-water (10:2; 12 mL) was added 30% aqueous H₂O₂ (10 mL) at 0° C. and the resulting reaction mixture was stirred at room temperature for 2 h. Lithium hydroxide monohydrate (0.230 g, 5.51 mmol) was added into the reaction solution at 0° C. and stirring was further continued at room temperature for 10 minutes. [One more batch (2.5 g) was performed and combined with the present batch prior to work-up]. Combined mixtures were backwashed with ethyl acetate (30 mL×2), separated aqueous layer was acidified (pH ~4) with formic acid and was extracted with 20% MeOH in DCM (100 mL×4). Collected organic parts were dried over anhydrous Na₂SO₄ and concentrated under reduced pressure to get (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (3.1 g, 87%; crude) as an off white solid. MS: [MH]⁺ 545.2.

Synthesis of Methyl 4-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoate: To a stirred solution of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (0.250 g, 0.45 mmol) in DCM (5 mL) were added methyl 4-(1-(O-((2-oxabicyclo[2.2.2]octan-4-yl)methyl)-L-threonyl)piperidin-4-yl)-2-hydroxybenzoate (0.310 g, 0.68 mmol) and DCC (0.190 g, 0.91 mmol) sequentially at 0° C. under nitrogen and the resulting mixture stirred at room temperature for 2 h. Reaction mixture was slowly poured into ice water (50 mL) and was extracted with DCM (50 mL×3). Combined organic extracts were wash with brine (20 mL) and dried over anhydrous Na₂SO₄ and concentrated under reduced pressure to get a crude product. The crude product was purified by silica gel column chromatography, using MeOH-DCM=0:1-1:9 as gradient, to afford methyl 4-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoate (0.400 g, 57%) as an off-white solid. MS: [MH]⁺ 987.6.

Synthesis of 4-(1-(O-((2-Oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid: To a stirred solution of methyl 4-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoate (0.350 g, 0.35 mmol) in a mixture of THF-water (3.5:1.5; 5 mL) was added lithium hydroxide monohydrate (0.044 g, 1.06 mmol) at room temperature and the resulting reaction mixture was stirred at 60° C. for 8 h. After cooling to room temperature, the reaction mass was backwashed with diethyl ether (50 mL×2), separated aqueous layer was acidified (pH ~2-3) with 1N HCl solution and was extracted with ethyl acetate (50 mL×3). Combined organic extracts were dried over anhydrous Na₂SO₄ and concentrated under reduced pressure to get a crude product, which was purified by prep-HPLC, 0.1% formic acid in water-acetonitrile as gradient, to afford 4-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid (0.070, 20%) as a white solid. ¹H-NMR (400 MHz, DMSO-d₆) δ 13.91 (br, 1H), 11.22 (br, 1H), 8.43-8.39 (m, 2H), 7.87-7.83 (d, J=16.0 Hz, 1H), 7.73-7.71 (m, 3H), 7.44-7.43 (d, J=6.4 Hz, 2H), 6.79 (brs, 2H), 5.48 (s, 2H), 4.88-4.83 (m, 1H), 4.52-4.50 (m, 1H), 4.39-3.41 (m, 14H), 3.23-2.67 (m, 4H), 2.50 (m, 4H; merged in DMSO-d₆), 1.93-1.69 (m, 3H), 1.54-1.01 (m, 13H). MS: [MH]⁺ 973.6.

### Synthesis of 2-(piperidin-4-yl)thiazole

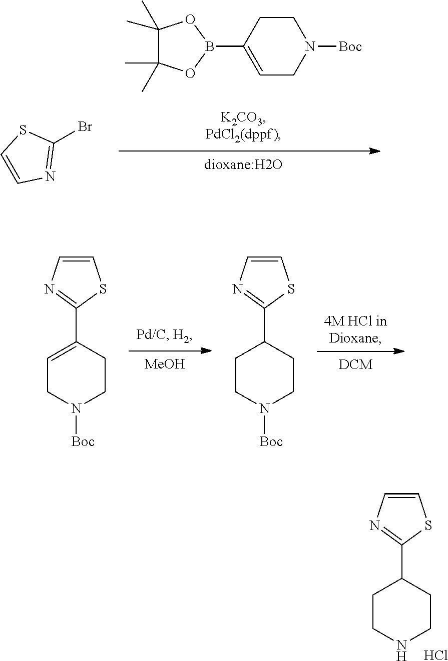

Synthesis of tert-Butyl 4-(thiazol-2-yl)-3,6-dihydropyridine-1 (2H)-carboxylate: To a stirred solution of 2-bromothiazole (4.0 g, 24.53 mmol) in a mixture of dioxane and water (4:1, 100 mL) were added tert-butyl 4-(4,4,5,5-tetramethyl-1,3,2-dioxaborolan-2-yl)-3,6-dihydropyridine-1 (2H)-carboxylate (9.09 g, 29.44 mmol) and K₂CO₃ (10.15 g, 73.61 mmol) sequentially at room temperature. The reaction mixture was degassed (purging with nitrogen) for 20 min followed by the addition of PdCl₂(dppf)(1.80 g, 2.43 mmol) and the reaction mixture was heated at 90° C. for 4 h. After cooling to room temperature, reaction was diluted with water (200 mL) and was extracted with diethyl ether (100 mL×3). Collected organic extracts were washed with brine (200 mL), dried over anhydrous Na₂SO₄ and concentrated under reduce pressure. The crude product was purified by silica gel column chromatography, using ethyl acetate-hexane=0:1→0.7:9.3 as gradient, to afford tert-butyl 4-(thiazol-2-yl)-3,6-dihydropyridine-1 (2H) (5.0 g, 80%) as a colourless oil. MS: [(M-56)⁺H]⁺ 267.1.

Synthesis of tert-Butyl 4-(thiazol-2-yl)piperidine-1-carboxylate: To a stirred solution of tert-butyl 4-(thiazol-2-yl)-3,6-dihydropyridine-1 (2H)-carboxylate (3.0 g, 11.27 mmol) in a methanol (30 mL) was added 10% Pd in activated carbon (1.3 g) at room temperature under nitrogen and the resulting mixture was hydrogenated under balloon pressure at the same temperature for 2 h. Reaction mixture was filter through a celite bed, washed the bed with methanol (100 mL) and collected filtrates were concentrated under reduced pressure to afford tert-butyl 4-(thiazol-2-yl)piperidine-1-carboxylate (2.8 g, 92%) as a colorless oil. MS: [(M-100)⁻H]⁺169.1.

Synthesis of 2-(Piperidin-4-yl)thiazole: To a stirred solution of tert-butyl 4-(thiazol-2-yl)piperidine-1-carboxylate (1.5 g, 5.59 mmol) in DCM (10.0 mL) was added 4M HCl in dioxane (9.0 mL) at 0° C. and stirred the solution at room temperature for 1 h. Reaction mixture was concentrated under reduced pressure to obtained crude product, which was purified by trituration using n-pentane, to afford 2-(piperidin-4-yl)thiazole hydrochloride (1.4 g, quantitative) as an off-white solid. MS: [MH]⁺ 169.1.

### Synthesis of afforded (S)—N-((2S,3R)-1-oxo-3-((tetrahydro-2H-pyran-4-yl)methoxy)-1-(4-(thiazol-2-yl)piperidin-1-yl)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (I-79)

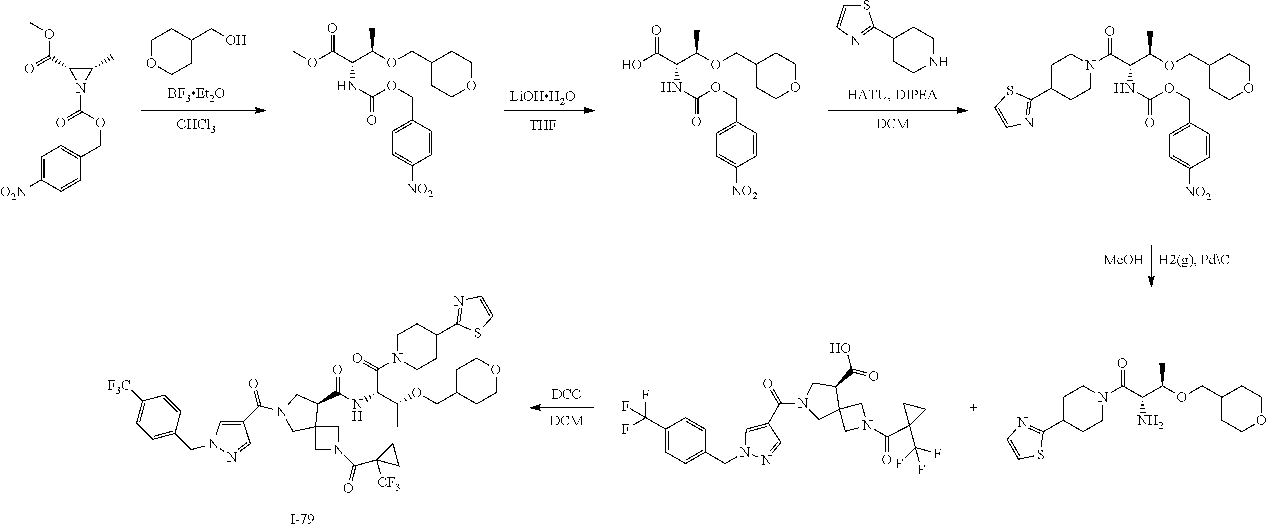

Synthesis of 2-methyl 1-(4-nitrobenzyl) (2S, 3S)-3-methylaziridine-1,2-dicarboxylate mentioned under Methyl 2-(1-(O-((2-oxabicyclo[2.2.2]octan-4-yl)methyl)-L-threonyl)piperidin-4-yl)-5-(trifluoromethyl) benzoate.

Synthesis of Methyl N-(((4-nitrobenzyl)oxy)carbonyl)-O-((tetrahydro-2H-pyran-4-yl)methyl)-L-threoninate: To a stirred solution of 2-methyl 1-(4-nitrobenzyl) (2S,3S)-3-methylaziridine-1,2-dicarboxylate (3.0 g, 10.20 mmol) in CHCl₃ (10 mL) was added (tetrahydro-2H-pyran-4-yl)methanol (1.42 g, 12.24 mmol) at 0° C. under nitrogen. After 5 min of stirring at the same temperature, was added BF₃-Et₂O (2.89 g, 20.40 mmol) drop-wise—into the reaction mass and allowed to stirred at room temperature for 3 h. [Another identical batch (3.0 g) was carried out in parallel and mixed together prior to work-up]. Combined reaction mixtures were concentrated under reduced pressure to get crude product, which was purified by silica gel column chromatography using EtOAc-hexane=3:7→4:6 as gradient, to afforded methyl N-(((4-nitrobenzyl)oxy)carbonyl)-O-((tetrahydro-2H-pyran-4-yl)methyl)-L-threoninate (5.1 g, 60%) as a yellow liquid. MS: MH]+ 411.3.

Synthesis of N-(((4-nitrobenzyl)oxy)carbonyl)-O-((tetrahydro-2H-pyran-4-yl)methyl)-L-threonine: To a stirred solution of methyl N-(((4-nitrobenzyl)oxy)carbonyl)-O-((tetrahydro-2H-pyran-4-yl)methyl)-L-threoninate (3.4 g, 8.29 mmol) in THF (20 mL) was added an aqueous (6 mL) solution of LiOH·H₂O (1.39 g, 33.17 mmol) at room temperature and allowed to stir for 5 h. [Another batch of 2.7 g was performed in parallel and mixed together prior to work-up]. Combined reaction mixtures was back washed by diethyl ether (200 mL), separated aqueous layer was acidified (pH=2-3) with an aqueous solution of 1N HCl and was extracted with ethyl acetate (200 mL×2). Collected organic extracts were dried over anhydrous Na₂SO₄ and concentrated under reduced pressure to afforded N-(((4-nitrobenzyl)oxy)carbonyl)-O-((tetrahydro-2H-pyran-4-yl)methyl)-L-threonine (4.5 g, 91%; crude) as a yellow oil, which was used in next step without purification. MS: [MH]+397.3.

Synthesis of 4-Nitrobenzyl ((2S,3R)-1-oxo-3-((tetrahydro-2H-pyran-4-yl)methoxy)-1-(4-(thiazol-2-yl)piperidin-1-yl)butan-2-yl)carbamate: To a stirred solution of N-(((4-nitrobenzyl)oxy)carbonyl)-O-((tetrahydro-2H-pyran-4-yl)methyl)-L-threonine (1.48 g, 3.73 mmol) in DCM (10 mL), was added DIPEA (1.36 g, 10.605 mmol) at room temperature. The resulting reaction mixture cooled at 0° C. followed by addition of HATU (2.13 g, 5.60 mmol). After additional stirring for 10 min at the same temperature, was added 2-(piperidin-4-yl)thiazole (0.940 g, 5.60 mmol) was added at 0° C. temperature and stirring was continued at room temperature for 3 h. Reaction mixture was diluted with water (200 mL) and was extracted with DCM (200×2 mL). Combined organic extracts were dried over anhydrous Na₂SO₄ and concentrated under reduced pressure to get a crude product, which was purified by silica gel column chromatography using MeOH-DCM=0:1→0.5:9.5 as gradient, to afforded 4-nitrobenzyl ((2S,3R)-1-oxo-3-((tetrahydro-2H-pyran-4-yl)methoxy)-1-(4-(thiazol-2-yl)piperidin-1-yl)butan-2-yl)carbamate (1.30 g, 67%) as light yellow liquid. MS: [MH]⁺ 547.3.

Synthesis of (2S,3R)-2-amino-3-((tetrahydro-2H-pyran-4-yl)methoxy)-1-(4-(thiazol-2-yl)piperidin-1-yl)butan-1-one: To a stirred solution of 4-nitrobenzyl ((2S,3R)-1-oxo-3-((tetrahydro-2H-pyran-4-yl)methoxy)-1-(4-(thiazol-2-yl)piperidin-1-yl)butan-2-yl)carbamate (1.3 g, 2.38 mmol) in methanol (10 mL) was added Pd/C (0.600 g) at room temperature under nitrogen. The reaction mixture was degassed (purging with nitrogen) for 5 min followed by hydrogenated under balloon pressure at room temperature. Reaction mixture was filtered through celite bed, washed the bed with MeOH (200 mL) and collected filtrates were concentrated under reduced pressure to get crude product. The resulting crude was purified by silica gel column chromatography using DCM-MeOH=0.5:9.5→0.9:9.1 as gradient, to afforded (2S,3R)-2-amino-3-((tetrahydro-2H-pyran-4-yl)methoxy)-1-(4-(thiazol-2-yl)piperidin-1-yl)butan-1-one (0.450 g, 51%) as yellow liquid. MS: [MH]+ 368.3.

Synthesis of (S)—N-((2S,3R)-1-oxo-3-((tetrahydro-2H-pyran-4-yl)methoxy)-1-(4-(thiazol-2-yl)piperidin-1-yl)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide: To a stirred solution of (2S,3R)-2-amino-3-((tetrahydro-2H-pyran-4-yl)methoxy)-1-(4-(thiazol-2-yl)piperidin-1-yl)butan-1-one (0.390 g, 0.96 mmol) in DCM (5 mL) was added (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (0.350 g, 0.64 mmol) at room temperature. After reaction became homogenous, reaction was brought to 0° C. followed by addition of DCC (0.260 g, 1.28 mmol) into the reaction solution and stirred for 2 h at room temperature. Reaction mixture was diluted with water (100 mL) and was extracted by DCM (150×3 mL). Combined organic extracts were dried over anhydrous Na₂SO₄ and concentrated under reduced pressure to get crude product, which was purified by silica gel column chromatography using MeOH-DCM=0.5:9.5→0.9:9.1 as gradient, to afforded (S)—N-((2S,3R)-1-oxo-3-((tetrahydro-2H-pyran-4-yl)methoxy)-1-(4-(thiazol-2-yl)piperidin-1-yl)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (1-79) (0.250 g, 43%) as an off white solid. ¹H NMR (400 MHz, DMSO-d₆) δ 8.46-8.42 (m, 2H), 7.88-7.84 (m, 1H), 7.73-7.72 (d, J=7.6 Hz, 3H), 7.63-7.57 (m, 1H), 7.45-7.43 (d, J=8.0 Hz, 2H), 5.48 (s, 2H), 4.94 (br. s, 1H), 4.41 (m, 1H), 4.17-3.51 (m, 11H), 3.26-3.16 (m, 5H), 2.81-2.60 (m, 1H), 2.09 (m, 2H), 1.69-1.05 (m, 16H). (There is short count of 2 protons in aliphatic region, that could be merged with either moisture or DMSO-d₆ signals); MS: [MH]⁺: 894.62.

### Synthesis of 4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoic acid as (I-64)

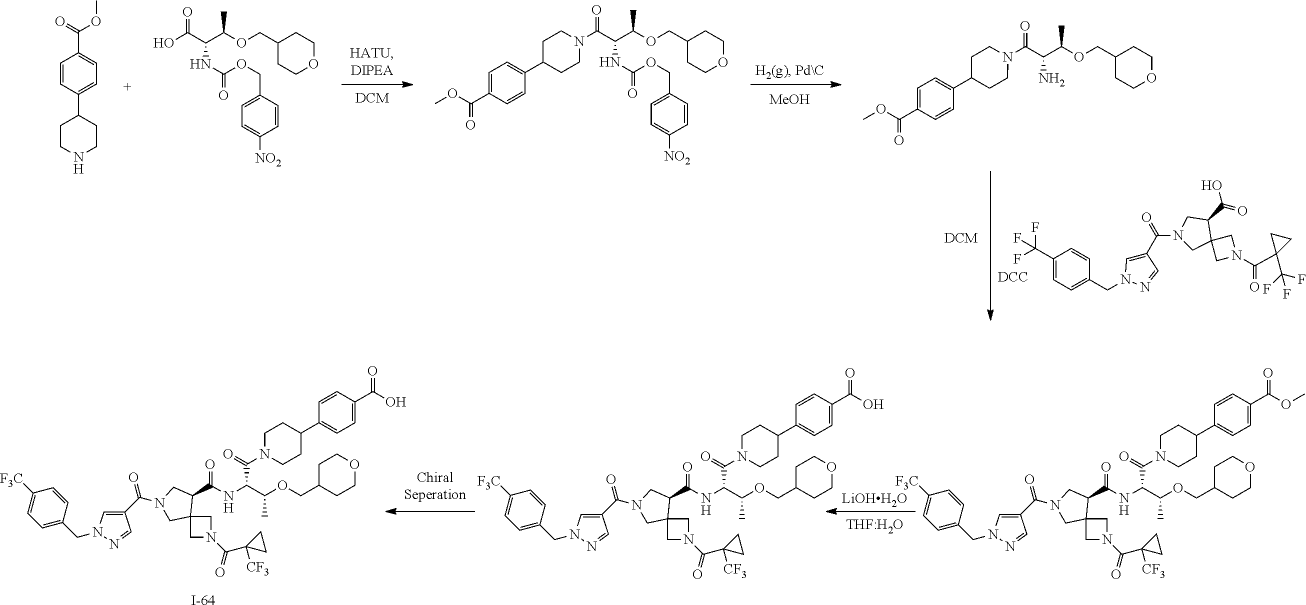

Synthesis of N-(((4-nitrobenzyl)oxy)carbonyl)-O-((tetrahydro-2H-pyran-4-yl)methyl)-L-threonine was mentioned under (S)—N-((2S,3R)-1-oxo-3-((tetrahydro-2H-pyran-4-yl)methoxy)-1-(4-(thiazol-2-yl)piperidin-1-yl)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (I-79)

Synthesis of Methyl4-(1-(N-(((4-nitrobenzyl)oxy)carbonyl)-O-((tetrahydro-2H-pyran-4-yl)methyl)-L-threonyl)piperidin-4-yl)benzoate To a stirred solution of N-(((4-nitrobenzyl)oxy)carbonyl)-O-((tetrahydro-2H-pyran-4-yl)methyl)-L-threonine (1.20, 3.03 mmol) in DCM (12 mL) was added DIPEA (1.17 g, 9.09 mmol) and HATU (2.4 g, 6.31 mmol) at 0° C. under nitrogen. After 10 minutes of stirring at the same temperature, was added methyl 4-(piperidin-4-yl)benzoate (1.0 g, 4.54 mmol) and stirring was continued for 1 h at room temperature. Reaction mixture was slowly poured into ice water (500 mL) and was extracted with DCM (200 mL×3). Organic extracts washed with brine (400 mL), dried over anhydrous Na₂SO₄ and concentrated under reduced pressure to get a crude product. The crude product was purified by silica gel column chromatography, using ethyl acetate-hexane=0:1→3:2 to afford methyl4-(1—(N-(((4-nitrobenzyl)oxy)carbonyl)-O-((tetrahydro-2H-pyran-4-yl)methyl)-L-threonyl)piperidin-4-yl)benzoate (1.20 g, 66%) as an off-white solid. MS: [MH]⁺ 598.4.

Methyl 4-(1-(O-((tetrahydro-2H-pyran-4-yl)methyl)-L-threonyl)piperidin-4-yl)benzoate To a stirred solution of methyl4-(1-(N-(((4-nitrobenzyl)oxy)carbonyl)-O-((tetrahydro-2H-pyran-4-yl)methyl)-L-threonyl)piperidin-4-yl)benzoate (1.0 g, 1.67 mmol) in methanol (10 mL), was added 10% Pd on activated carbon (0.500 g) at room temperature and the resulting reaction mixture was hydrogenated under balloon pressure at room temperature for 3 h. Reaction mixture was filtered through celite bed, washed the bed with methanol (300 mL) and collected filtrates were concentrated under reduced pressure to get a crude product. The crude product was purified by silica gel column chromatography, using (MeOH-DCM=0:1→1:19) as an eluent, to afforded methyl 4-(1-(O-(tetrahydro-2H-pyran-4-yl)methyl)-L-threonyl)piperidin-4-yl)benzoate (0.700 g, 100%) as an oily liquid. MS: [MH]⁺ 419.4.

### Synthesis of (S)-6-(1-(4-(Trifluoromethyl)benzyl)-1H-pyrazole-4-carbonyl)-2-(1 (trifluoromethyl)cyclopropane-1-carbonyl)-2,6-diazaspiro[3.4]octane-8-carboxylic acid was mentioned under 4-(1-(O-((2-Oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid (I-81)

Synthesis of Methyl 4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoate a stirred solution of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (0.100 g, 0.18 mmol) in DCM (1 mL) were added methyl 4-(1-(O-(tetrahydro-2H-pyran-4-yl)methyl)-L-threonyl)piperidin-4-yl)benzoate (0.076 g, 00.18 mmol) and DCC (0.076 g, 0.36 mmol) at 0° C. under nitrogen. The resulting reaction mixture stirred for 30 minutes at room temperature. [Another identical batch of 0.400 g was performed and worked up together]. The reaction mixture was slowly poured into ice water (40 mL) and was extracted with DCM (50 mL×3). Combined organic extracts were washed with brine (20 mL), dried over anhydrous Na₂SO₄ and concentrated under reduced pressure to get a crude product. Resulting crude was purified by reverse phase (C-18) silica gel column chromatography using acetonitrile-water=0:1→6:4 as gradient, to afford methyl 4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoate (0.400 g, 58%) as an off white solid. MS: [MH]⁻ 945.5.

Synthesis of 4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoic acid (I-64) To a stirred solution of methyl 4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoate (0.400 g, 0.42 mmol) in a mixture of THE-water (3:1; 6 mL) was added lithium hydroxide monohydrate (0.089 g, 2.11 mmol) at room temperature and the resulting reaction mixture stirred for 3 h at room temperature. The reaction mixture was concentrated under reduced pressure, obtained crude was diluted with water (40 mL) and it was extracted with diethyl ether (60 mL×2) to remove unwanted organic impurities. Aqueous part was acidified (pH ~2-3) with an aqueous solution of 1N HCl and was extracted with ethyl acetate (50 mL×3). Combined organic extracts were wash with brine (100 mL), dried over anhydrous Na₂SO₄ and concentrated under reduced pressure to get a crude product. The crude product was purified by prep-HPLC, 0.1% formic acid in water-acetonitrile/methanol/IPA as gradient, to afforded 4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoic acid (0.350 g, 89%) as an off-white solid. MS: [MH]⁺ 931.5. Note: Though Applicant started with chiral intermediates, they observed racemization in the final compound as observed by Chiral HPLC. So they further separated each enantiomer by chiral SFC.

4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoic acid (I-61 (A)) (0.035 g, 8%) as a white solid. ¹H-NMR (400 MHz, DMSO-d₆) δ 12.76 (br, 1H), 8.48-8.42 (m, 2H), 7.90-7.84 (m, 3H), 7.73-7.71 (d, J=7.6 Hz, 2H), 7.44-7.32 (m, 4H), 5.48 (s, 2H), 4.94 (brs, 1H), 4.54-3.34 (m, 12H), 3.29-2.67 (m, 7H), 1.97-1.54 (m, 10H), 1.16-1.04 (m, 7H), 0.86-0.82 (m, 1H). MS: [MH]⁺ 931.6.

4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoic acid (I-64 (B)) (0.030 g, 8%) as a white solid. ¹H-NMR (400 MHz, DMSO-d₆) δ 12.81 (br, 1H), 8.65-8.50 (m, 1H), 8.44-8.41 (d, J=11.2 Hz, 1H), 7.87-7.83 (m, 3H), 7.73-7.71 (d, J=7.6 Hz, 2H), 7.44-7.42 (d, J=7.6 Hz, 2H), 7.33 (brs, 2H), 5.48 (s, 2H), 4.86 (brs, 1H), 4.54 (brs, 1H), 4.20-3.47 (m, 11H), 3.20-3.10 (m, 3H), 2.90 (br, 1H), 2.67-2.57 (m, 2H), 1.97-1.81 (m, 3H), 1.72-1.51 (m, 5H), 1.41-1.11 (10H), 0.92-0.86 (m, 1H). MS: [MH]⁺ 931.6.

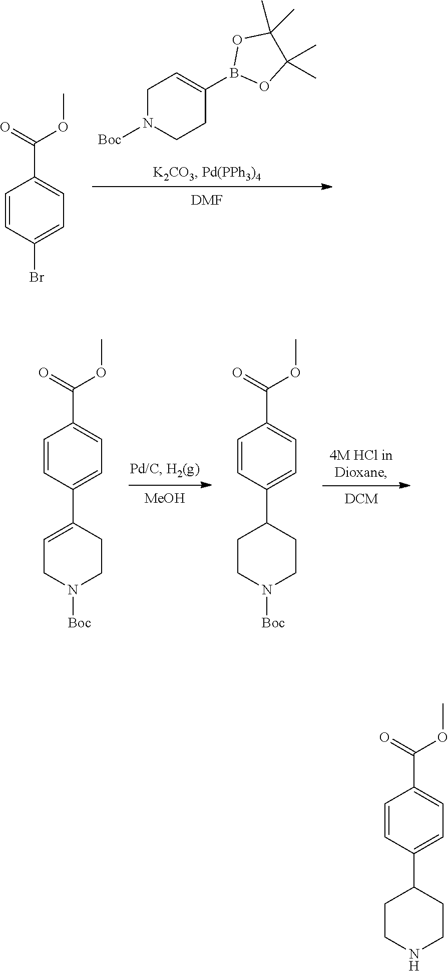

Synthesis of tert-Butyl 4-(4-(methoxycarbonyl)phenyl)-3,6-dihydropyridine-1 (2H)-carboxylate To a stirred solution of methyl 4-bromobenzoate (3.0 g, 13.95 mmol) in DMF (30 mL) were added tert-butyl 4-(4,4,5,5-tetramethyl-1,3,2-dioxaborolan-2-yl)-3,6-dihydropyridine-1 (2H)-carboxylate (4.31 g, 13.95 mmol) and K₂CO₃ (5.77 g, 41.85 mmol) sequentially at room temperature. The reaction mixture was degassed (purging with nitrogen) for 20 min followed by the addition of Pd(PPh₃)₄ (0.805 g, 0.697 mmol) and the reaction mixture was heated at 90° C. for 4 h. After cooling to room temperature, reaction was diluted with water (200 mL) and was extracted with diethyl ether (100 mL×3). Collected organic extracts were washed with brine (200 mL), dried over anhydrous Na₂SO₄ and concentrated under reduce pressure. The crude product was purified by silica gel column chromatography, using ethyl acetate-hexane=0:1→0.7:9.3 as gradient, to afford tert-Butyl 4-(4-(methoxycarbonyl)phenyl)-3,6-dihydropyridine-1 (2H)-carboxylate (3.20 g, 69%) as a colorless oil. MS: [MH]⁺ 318.1.

Synthesis of tert-Butyl 4-(4-(methoxycarbonyl)phenyl)piperidine-1-carboxylate To a stirred solution of tert-Butyl 4-(4-(methoxycarbonyl)phenyl)-3,6-dihydropyridine-1 (2H)-carboxylate (3.2 g, 10.09 mmol) in a methanol (28 mL) was added 10% Pd in activated carbon (2.0 g) at room temperature under nitrogen and the resulting mixture was hydrogenated under balloon pressure at the same temperature for 2 h. Reaction mixture was filter through a celite bed, washed the bed with methanol (100 mL) and collected filtrates were concentrated under reduced pressure to afford tert-Butyl 4-(4-(methoxycarbonyl)phenyl)piperidine-1-carboxylate (2.70 g, 86%) as a colorless oil. MS: [MH]⁺ 220.1.

Synthesis of Methyl 4-(piperidin-4-yl)benzoate To a stirred solution of tert-Butyl 4-(4-(methoxycarbonyl)phenyl)piperidine-1-carboxylate (2.70 g, 8.46 mmol) in DCM (25.0 mL) was added 4M HCl in dioxane (10.0 mL) at 0° C. and stirred the solution at room temperature for 1 h. Reaction mixture was concentrated under reduced pressure to obtained crude product, which was purified by trituration using n-pentane, to afford methyl 4-(piperidin-4-yl)benzoate hydrochloride (2.20 g, quantitative) as an off-white solid. MS: [MH]⁺ 169.1.

### Synthesis of (S)-6-(5-hydroxypyrazine-2-carbonyl)-N-((2S,3R)-1-oxo-3-((tetrahydro-2H-pyran-4-yl)methoxy)-1-(4-(trifluoromethyl)piperidin-1-yl)butan-2-yl)-2-(1-(trifluoromethyl)cyclopropane-1-carbonyl)-2,6-diazaspiro[3.4]octane-8-carboxamide (I-78)

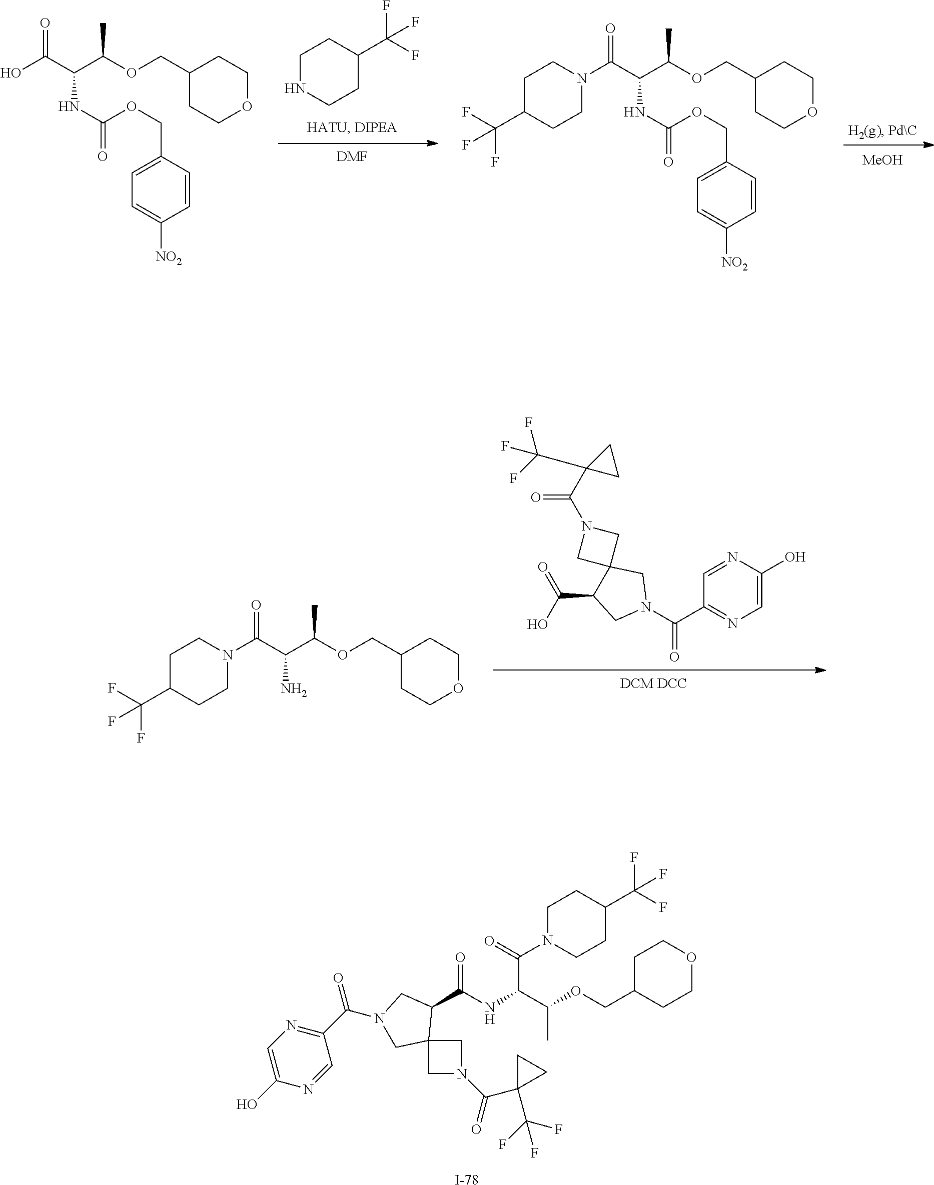

Synthesis of 4-Nitrobenzyl ((2S,3R)-1-oxo-3-((tetrahydro-2H-pyran-4-yl)methoxy)-1-(4-(trifluoromethyl)piperidin-1-yl)butan-2-yl)carbamate To a stirred solution of N-(((4-nitrobenzyl)oxy)carbonyl)-O-((tetrahydro-2H-pyran-4-yl)methyl)-L-threonine 1.30 g, 3.282 mmol) in DCM (10 mL) was added DIPEA (0.820 g, 6.56 mmol) and HATU (1.87 g, 4.92 mmol) at 0° C. under nitrogen. After 10 min of stirring at the same temperature, was added 4-(trifluoromethyl)piperidine (0.48 g, 3.282 mmol) and stirring was continued for 1 h at room temperature. Reaction mixture was slowly poured into ice water (50 mL) and was extracted with ethyl acetate (50 mL×3). Combined organic extracts were washed with brine (20 mL), dried over anhydrous Na₂SO₄ and concentrated under reduced pressure to get a crude product. The crude product was purified by column chromatography on silica gel using MeOH-DCM=0:1-1:19 to afford 4-nitrobenzyl ((2S,3R)-1-oxo-3-((tetrahydro-2H-pyran-4-yl)methoxy)-1-(4-(trifluoromethyl)piperidin-1-yl)butan-2-yl)carbamate (1.0 g, 57%) as a colourless gummy oil. MS: [MH]⁻ 532.5.

Synthesis of (2S,3R)-2-amino-3-((tetrahydro-2H-pyran-4-yl)methoxy)-1-(4-(trifluoromethyl)piperidin-1-yl)butan-1-one 10% Pd in activated carbon (0.500 g) was added carefully to a stirred solution of 4-nitrobenzyl ((2S,3R)-1-oxo-3-((tetrahydro-2H-pyran-4-yl)methoxy)-1-(4-(trifluoromethyl)piperidin-1-yl)butan-2-yl)carbamate (1.0 g, 1.88 mmol) in a methanol (10 mL) at room temperature under nitrogen and the resulting mixture was hydrogenated under balloon pressure at the same temperature for 2 h. The reaction mixture was filtered through a celite bed, washed the bed with methanol (30 mL×2) and collected filtrates were concentrated under reduced pressure. The crude product was purified by column chromatography on silica gel using MeOH:DCM=0:141:19 as eluent, to afford (2S,3R)-2-amino-3-((tetrahydro-2H-pyran-4-yl)methoxy)-1-(4-(trifluoromethyl)piperidin-1-yl)butan-1-one (0.420 g, 67%) as a colourless gummy. MS: [MH]⁺353.5.

Synthesis of (S)-6-(5-hydroxypyrazine-2-carbonyl)-N-((2S,3R)-1-oxo-3-((tetrahydro-2H-pyran-4-yl)methoxy)-1-(4-(trifluoromethyl)piperidin-1-yl)butan-2-yl)-2-(1-(trifluoromethyl)cyclopropane-1-carbonyl)-2,6-diazaspiro[3.4]octane-8-carboxamide To a stirred solution of (S)-6-(5-hydroxypyrazine-2-carbonyl)-2-(1-(trifluoromethyl)cyclopropane-1-carbonyl)-2,6-diazaspiro[3.4]octane-8-carboxylic acid (0.300 g, 0.72 mmol) in DCM (5 mL) were added (2S,3R)-2-amino-3-((tetrahydro-2H-pyran-4-yl)methoxy)-1-(4-(trifluoromethyl)piperidin-1-yl)butan-1-one (0.382 g, 1.086 mmol) and DCC (0.298 g, 1.448 mmol) sequentially at 0° C. under nitrogen and the resulting reaction mixture was stirred for 2 h at room temperature. Reaction mixture was slowly poured into ice water (20 mL) and was extracted with DCM (30 mL×3). Combined organic extracts were washed with brine (20 mL), dried over anhydrous Na₂SO₄ and concentrated under reduced pressure to get a crude product. The crude product was purified by Prep HPLC, 0.1% formic acid in water-acetonitrile as a gradient, to afford (S)-6-(5-hydroxypyrazine-2-carbonyl)-N-((2S,3R)-1-oxo-3-((tetrahydro-2H-pyran-4-yl)methoxy)-1-(4-(trifluoromethyl)piperidin-1-yl)butan-2-yl)-2-(1-(trifluoromethyl)cyclopropane-1-carbonyl)-2,6-diazaspiro[3.4]octane-8-carboxamide (0.080 g, 15%) as a white solid. ¹H-NMR (400 MHz, DMSO-d₆) δ 12.83 (br, 1H), 8.50-8.43 (m, 1H), 7.93-7.89 (m, 2H), 4.88 (br, 1H), 4.51-4.46 (m, 2H), 4.25-3.97 (m, 5H), 3.82-3.80 (d, J=8.8 Hz, 4H), 3.60 (br, 3H), 3.34-3.07 (m, 5H), 2.60-2.55 (m, 3H), 1.90-1.52 (m, 5H), 1.23-1.03 (m, 10H). MS: [MH]⁺ 749.4.

### Synthesis of (S)-6-(3-hydroxypyrazine-2-carbonyl)-N-((2S,3R)-1-oxo-1-(6-azaspiro[2.5]octan-6-yl)-3-((tetrahydro-2H-pyran-4-yl)methoxy)butan-2-yl)-2-(1-(trifluoromethyl)cyclopropane-1-carbonyl)-2,6-diazaspiro[3.4]octane-8-carboxamide (I-77)

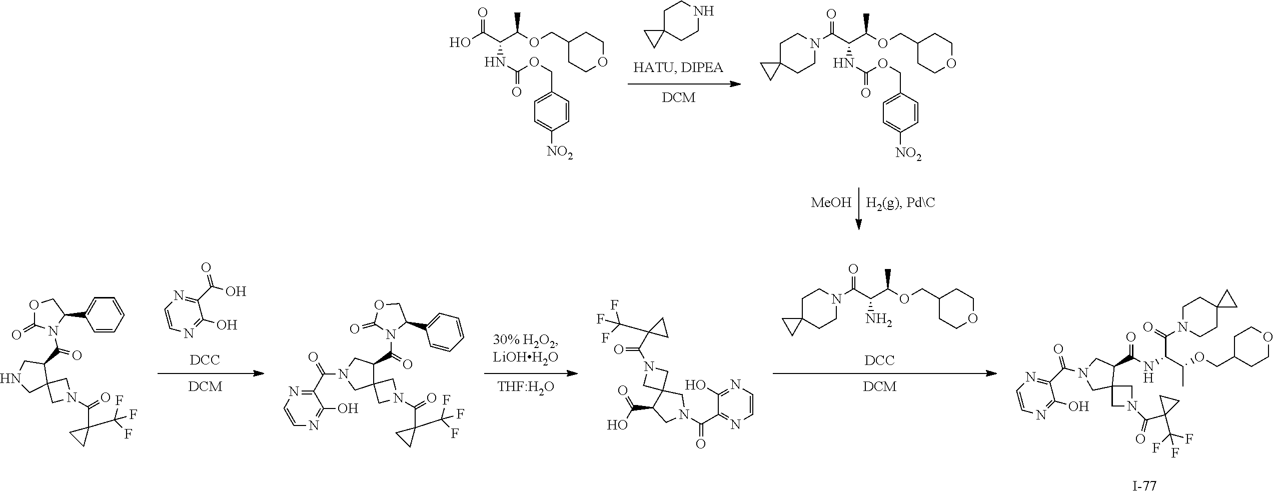

Synthesis of ((R)-3-((S)-6-(3-hydroxypyrazine-2-carbonyl)-2-(1-(trifluoromethyl)cyclopropane-1-carbonyl)-2,6-diazaspiro[3.4]octane-8-carbonyl)-4-phenyloxazolidin-2-one To a stirred solution of 3-hydroxypyrazine-2-carboxylic acid (1.0 g, 7.14 mmol) in DCM (40 mL) were added (R)-4-phenyl-3-((S)-2-(1-(trifluoromethyl)cyclopropane-1-carbonyl)-2,6-diazaspiro[3.4]octane-8-carbonyl)oxazolidin-2-one (2.5 g, 5.71 mmol) and DCC (2.9 g, 14.28 mmol) sequentially at room temperature under nitrogen and stirring was continued for 2 h at the same temperature. Reaction mixture was slowly poured into ice water (50 mL) and was extracted with ethyl acetate (100 mL×2). Combined organic extracts were washed with brine (150 mL), dried over anhydrous Na₂SO₄ and concentrated under reduced pressure to get a crude product, which was purified by column chromatography on silica gel, using MeOH-DCM=0:1-41:19 as eluent, to afford (R)-3-((S)-6-(3-hydroxypyrazine-2-carbonyl)-2-(1-(trifluoromethyl)cyclopropane-1-carbonyl)-2,6-diazaspiro[3.4]octane-8-carbonyl)-4-phenyloxazolidin-2-one (3.7 g, 93%) as a yellow solid. MS: [MH]⁺ 560.3.

Synthesis of (S)-6-(3-hydroxypyrazine-2-carbonyl)-2-(1-(trifluoromethyl)cyclopropane-1-carbonyl)-2,6-diazaspiro[3.4]octane-8-carboxylic acid To a stirred solution of (R)-3-((S)-6-(3-hydroxypyrazine-2-carbonyl)-2-(1-(trifluoromethyl)cyclopropane-1-carbonyl)-2,6-diazaspiro[3.4]octane-8-carbonyl)-4-phenyloxazolidin-2-one (1.0 g, 1.78 mmol) in a mixture of THF-water (5:1; 6 mL) was added H₂O₂ (30% in water; 8.0 mL) at 0° C. and the resulting mixture was stirred at room temperature for 2 h. After 2 h of stirring, reaction was cooled to 0° C. and was added LiOH:H₂O (0.142 g, 3.39 mmol) and stirred at room temperature for an additional 1 h. Reaction was diluted with water (10 mL) and was washed with ethyl acetate (30 mL) to get rid of unwanted organic impurities. Separated aqueous layer was acidified (pH ~2-3) with an aqueous solution of 1N HCl and was extracted with 20% IPA-CHCl₃ solution (100 mL×3). Combined organic extracts was dried over anhydrous Na₂SO₄ and concentrated under reduced pressure to afford (S)-6-(3-hydroxypyrazine-2-carbonyl)-2-(1-(trifluoromethyl)cyclopropane-1-carbonyl)-2,6-diazaspiro[3.4]octane-8-carboxylic acid (0.360 g, 48%) as an off white solid. MS: [MH]⁺ 415.2.

Synthesis of 4-Nitrobenzyl ((2S,3R)-1-oxo-1-(6-azaspiro[2.5]octan-6-yl)-3-((tetrahydro-2H-pyran-4-yl)methoxy)butan-2-yl)carbamate To a stirred solution of N-(((4-nitrobenzyl)oxy)carbonyl)-O-((tetrahydro-2H-pyran-4-yl)methyl)-L-threonine (1.0 g, 2.52 mmol) in DCM (10 mL) were added DIPEA (0.97 g, 7.57 mmol) and HATU (2.39 g, 6.31 mmol) at 0° C. under nitrogen. After 10 min of stirring at the same temperature, was added 6-azaspiro[2.5]octane (0.45 g, 3.03 mmol) and stirring was continued for 2 h at room temperature. Reaction mixture was slowly poured into ice water (50 mL) and was extracted with ethyl acetate (50 mL×3). Combined organic extracts were washed with brine (20 mL), dried over anhydrous Na₂SO₄ and concentrated under reduced pressure to get a crude product. The crude product was purified by column chromatography on silica gel using MeOH-DCM=0:1→1:19 as an eluent, to afford 4-nitrobenzyl ((2S,3R)-1-oxo-1-(6-azaspiro[2.5]octan-6-yl)-3-((tetrahydro-2H-pyran-4-yl)methoxy)butan-2-yl)carbamate (0.800 g, 66%) as a colourless gummy oil. MS: [MH]⁺ 490.3.

Synthesis of (2S,3R)-2-amino-1-(6-azaspiro[2.5]octan-6-yl)-3-((tetrahydro-2H-pyran-4-yl)methoxy)butan-1-one 10% Pd on activated carbon (0.300 g) was added carefully to a stirred solution of 4-nitrobenzyl ((2S,3R)-1-oxo-1-(6-azaspiro[2.5]octan-6-yl)-3-((tetrahydro-2H-pyran-4-yl)methoxy)butan-2-yl)carbamate (0.700 g, 1.43 mmol) in a methanol (8 mL) at room temperature under nitrogen and the resulting mixture was hydrogenated under balloon pressure for 2 h at the same temperature. Reaction mixture was filtered through a celite bed, washed the bed with methanol (30 mL×2) and collected filtrates were concentrated under reduced pressure. The crude product was purified by column chromatography on silica gel using MeOH:DCM=0:1→1:19 to afford (2S,3R)-2-amino-1-(6-azaspiro[2.5]octan-6-yl)-3-((tetrahydro-2H-pyran-4-yl)methoxy)butan-1-one (0.450 g, 88%) as a colourless gummy oil. MS: [MH]⁺311.3.

Synthesis of (S)-6-(3-hydroxypyrazine-2-carbonyl)-N-((2S,3R)-1-oxo-1-(6-azaspiro[2.5]octan-6-yl)-3-((tetrahydro-2H-pyran-4-yl)methoxy)butan-2-yl)-2-(1-(trifluoromethyl)cyclopropane-1-carbonyl)-2,6-diazaspiro[3.4]octane-8-carboxamide (1-77) To a stirred solution of (S)-6-(3-hydroxypyrazine-2-carbonyl)-2-(1-(trifluoromethyl)cyclopropane-1-carbonyl)-2,6-diazaspiro[3.4]octane-8-carboxylic acid (0.350 g, 0.84 mmol) in DCM (6 mL) were added (2S,3R)-2-amino-1-(6-azaspiro[2.5]octan-6-yl)-3-((tetrahydro-2H-pyran-4-yl)methoxy)butan-1-one (0.392 g, 1.26 mmol) and DCC (0.348 g, 1.69 mmol) at 0° C. under nitrogen and the resulting reaction mixture was stirred for 2 h at room temperature. The reaction mixture was slowly poured into ice water (20 mL) and was extracted with DCM (30 mL×3). Combined organic extracts were washed with brine (20 mL), dried over anhydrous Na₂SO₄ and concentrated under reduced pressure to get a crude product. The crude product was purified by Prep HPLC, using 0.1% formic acid in water-acetonitrile as a gradient, to afford (S)-6-(3-hydroxypyrazine-2-carbonyl)-N-((2S,3R)-1-oxo-1-(6-azaspiro[2.5]octan-6-yl)-3-((tetrahydro-2H-pyran-4-yl)methoxy)butan-2-yl)-2-(1-(trifluoromethyl)cyclopropane-1-carbonyl)-2,6-diazaspiro[3.4]octane-8-carboxamidee (I-77) (0.070 g, 11%) as a white solid. ¹H-NMR (400 MHz, DMSO-d₆) δ 12.69 (brs, 1H), 8.48-8.38 (m, 1H), 7.57 (brs, 1H), 7.38 (brs, 1H), 4.89 (br. s, 1H), 4.40-4.02 (m, 3H), 3.82-3.79 (d, J=8.8 Hz, 3H), 3.63-3.46 (m, 9H), 3.27-3.18 (m, 4H), 2.60-2.45 (m, 3H, merged in DMSO-d₆), 1.68 (brs, 1H), 1.55-1.52 (d, J=13.2 Hz, 2H), 1.32-1.02 (m, 12H), 0.35-0.33 (m, 3H). MS: [MH]⁺ 707.4. MS: [MH]⁺ 707.4.

### Synthesis of (S)—N-((2S,3R)-1-oxo-1-(2-oxa-7-azaspiro[3.5]nonan-7-yl)-3-((tetrahydro-2H-pyran-4-yl)methoxy)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (I-67)

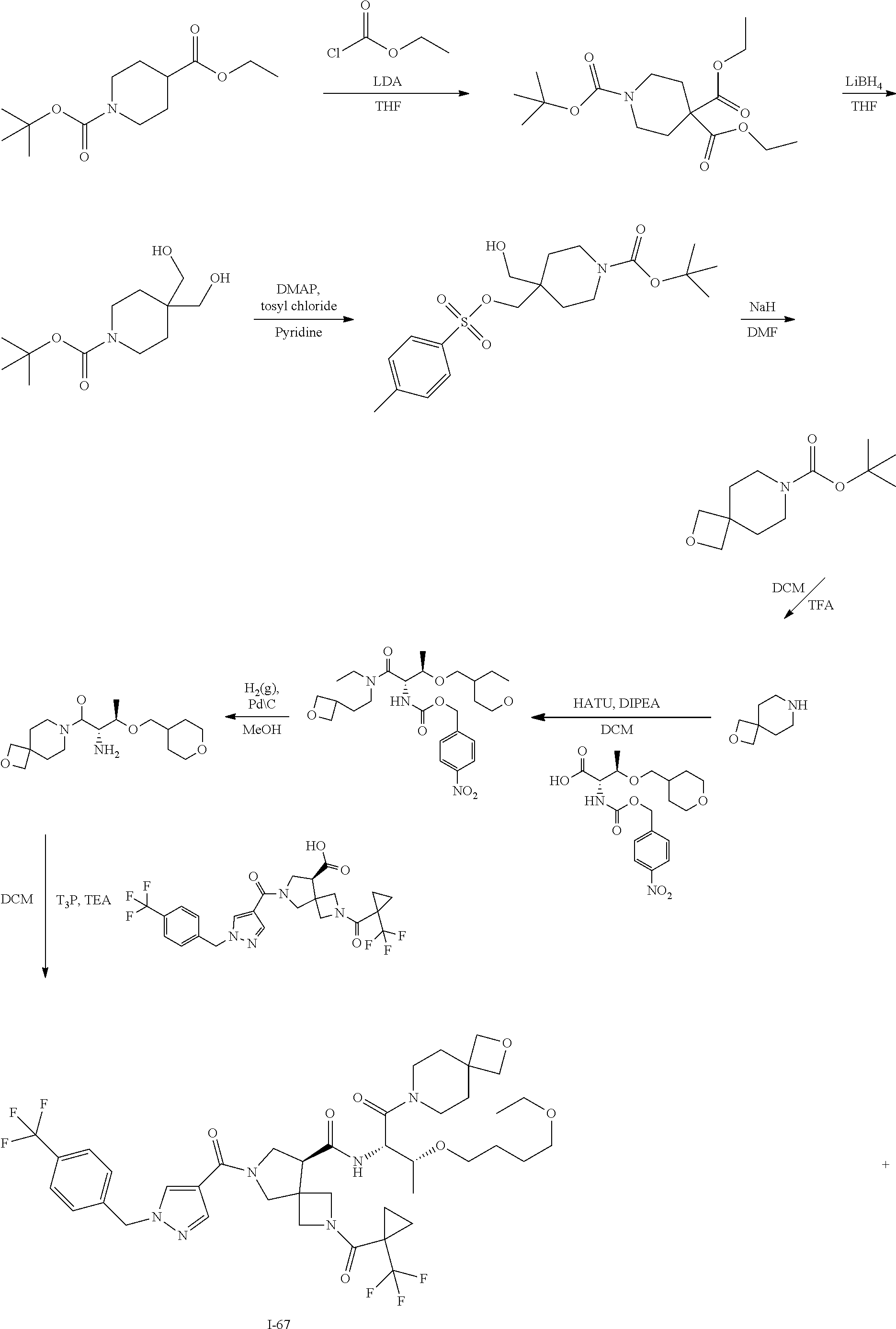

Synthesis of 1-(tert-Butyl) 4,4-diethyl piperidine-1,4,4-tricarboxylate To a stirred solution of 1-(tert-butyl) 4-ethyl piperidine-1,4-dicarboxylate (5.0 g, 19.40 mmol) in dry THF (10 mL) was added LDA (11.6 mL, 23.2 mmol) drop-wise at −78° C. under nitrogen over period of 30 min. After stirring for 1 h at the same temperature, was added ethyl chloroformate (2.10 g, 19.4 mmol) into the reaction mixture and stirring was continued for an additional 2 h at −78° C. Reaction mixture was slowly brought to room temperature, quenched with an aqueous solution of saturated ammonium chloride (100 mL) and was extracted with ethyl acetate (100 mL×3). Combined organic extracts were dried over anhydrous Na₂SO₄ and concentrated under reduce pressure to give 1-(tert-butyl) 4,4-diethyl piperidine-1,4,4-tricarboxylate (7.0 g, quant.; crude) as a yellow oil. MS: [MH-100]⁺ 230.20.

Synthesis of tert-Butyl 4,4-bis(hydroxymethyl)piperidine-1-carboxylate To a stirred solution of 1-(tert-butyl) 4,4-diethyl piperidine-1,4,4-tricarboxylate (7.0 g, 21.2 mmol) in a dry THF (30 mL) was added lithium borohydride (88 mL, 170.00 mmol) drop-wise at −78° C. over period of 30 min. The reaction mixture was heated at 60° C. for 48 h. Reaction mixture was cooled to room temperature, diluted with water (100 mL) and was extracted with ethyl acetate (100 mL×3). Combined organic extracts were dried over anhydrous Na₂SO₄ and concentrated under reduce pressure to obtained crude product. The crude product was purified by reverse-phase column chromatography, using water-acetonitrile=0:10→8:2 as a gradient, to afford tert-butyl 4,4-bis(hydroxymethyl)piperidine-1-carboxylate (3.2 g, 61%) as a white solid. MS: [MH-100]⁺ 146.10.

Synthesis of tert-Butyl 4-(hydroxymethyl)-4-((tosyloxy)methyl) piperidine-1-carboxylate To a stirred solution of tert-butyl 4,4-bis(hydroxymethyl)piperidine-1-carboxylate (2.20 g, 8.90 mmol) in pyridine (20 mL) was added DMAP (0.109 g, 0.89 mmol) at 0° C. under nitrogen. After stirring for 30 minutes at same temperature, was added tosyl-chloride (1.7 g, 8.90 mmol) and stirring was continued at room temperature for 16 h. Reaction mixture was diluted with water (100 mL) and was extracted with ethyl acetate (100 mL×3). Combined organic extracts were dried over anhydrous Na₂SO₄ and concentrated under reduce pressure to afforded tert-butyl 4-(hydroxymethyl)-4-((tosyloxy)methyl) piperidine-1-carboxylate (2.50 g, 70%; crude) as a yellow oil MS: [MH-100]⁺ 300.20.

Synthesis of tert-Butyl 2-oxa-7-azaspiro[3.5]nonane-7-carboxylate To a stirred solution of tert-butyl 4-(hydroxymethyl)-4-((tosyloxy)methyl) piperidine-1-carboxylate (2.50 g, 6.26 mmol) in DMF (20 mL) was added NaH (60% dispersion on oil; 0.751 g, 18.70 mmol) portion wise at 0° C. under nitrogen and the reaction mixture was stirred at room temperature for 16 h. Reaction mixture was diluted with cooled water (100 mL) and was extracted with ethyl acetate (100 mL×3). Combined organic extracts were dried over anhydrous Na₂SO₄ and concentrated under reduce pressure to obtained crude product, which was purified by flash column chromatography, using ethyl acetate-hexane: =0:1→3:2 as a gradient, to afford tert-butyl 2-oxa-7-azaspiro[3.5]nonane-7-carboxylate (1.0 g, 70%) as a white solid. MS: [MH-56]⁺ 172.10.

Synthesis of 2-Oxa-7-azaspiro[3.5]nonane To a stirred solution of tert-butyl 2-oxa-7-azaspiro[3.5]nonane-7-carboxylate (1.0 g, 4.40 mmol) in DCM (5 mL) was added TFA (1.5 mL) at 0° C. and the resulting reaction mixture was stirred at room temperature for 1 h. The reaction mixture was concentrated under reduced pressure and the obtained crude product was purified by trituration with DCM-diethyl ether, to afforded 2-oxa-7-azaspiro[3.5]nonane (0.900 g, quant; crude) as a brown sticky solid. MS: [MH]⁺128.10.

### Synthesis of N-(((4-nitrobenzyl)oxy)carbonyl)-O-((tetrahydro-2H-pyran-4-yl)methyl)-L-threonine mentioned under (S)—N-((2S,3R)-1-oxo-3-((tetrahydro-2H-pyran-4-yl)methoxy)-1-(4-(thiazol-2-yl)piperidin-1-yl)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide, Intermediate

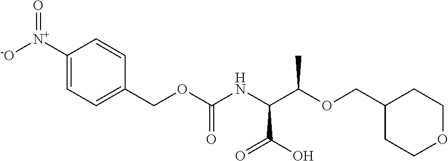

Synthesis of 4-Nitrobenzyl ((2S,3R)-1-oxo-1-(2-oxa-7-azaspiro[3.5]nonan-7-yl)-3-((tetrahydro-2H-pyran-4-yl)methoxy)butan-2-yl)carbamate To a stirred solution of N-(((4-nitrobenzyl)oxy)carbonyl)-O-((tetrahydro-2H-pyran-4-yl)methyl)-L-threonine (0.500 g, 1.26 mmol) in DCM (4 mL) were added DIPEA (0.488 g, 3.78 mmol) and HATU (0.718 g, 1.89 mmol) at 0° C. under nitrogen. After 10 min of stirring at the same temperature, was added 2-oxa-7-azaspiro[3.5]nonane (0.192 g, 1.51 mmol) and stirring was continued for 1.5 h at room temperature. [Another identical batch of 0.500 g was performed and worked up together]. Reaction mixture slowly poured in to water (100.0 mL) and was extracted with ethyl acetate (100 mL×3). Combined organic extracts were, dried over anhydrous Na₂SO₄ and concentrated under reduced pressure to get a crude product. The crude product was purified by silica gel column chromatography, using MeOH:DCM=0:1→1:4 as an eluent, to afforded 4-nitrobenzyl ((2S,3R)-1-oxo-1-(2-oxa-7-azaspiro[3.5]nonan-7-yl)-3-((tetrahydro-2H-pyran-4-yl)methoxy)butan-2-yl)carbamate (0.900 g, 70%) as a yellow sticky solid. MS: [MH]⁻ 506.3.

Synthesis of (2S,3R)-2-amino-1-(2-oxa-7-azaspiro[3.5]nonan-7-yl)-3-((tetrahydro-2H-pyran-4-yl)methoxy)butan-1-one 10% Pd on activated carbon (0.450 g) was added carefully to a stirred solution of 4-nitrobenzyl ((2S,3R)-1-oxo-1-(2-oxa-7-azaspiro[3.5]nonan-7-yl)-3-((tetrahydro-2H-pyran-4-yl)methoxy)butan-2-yl)carbamate (0.900 g, 1.78 mmol) in methanol (8 mL) at room temperature under nitrogen and the resulting mixture was hydrogenated under balloon pressure at the same temperature for 3 h. Reaction mixture was filtered through a celite bed, washed the bed with methanol (100 mL) and collected filtrates were concentrated under reduced pressure.

The crude product was purified by silica gel column chromatography, using MeOH:DCM=0:1→1:19 as an eluent, to afforded (2S,3R)-2-amino-1-(2-oxa-7-azaspiro[3.5]nonan-7-yl)-3-((tetrahydro-2H-pyran-4-yl)methoxy)butan-1-one (0.500 g, 86%) as a brown sticky mass. MS: [MH]⁺ 327.3.

### Synthetic procedure of (S)-6-(1-(4-(Trifluoromethyl)benzyl)-JH-pyrazole-4-carbonyl)-2-(1 (trifluoromethyl)cyclopropane-1-carbonyl)-2,6-diazaspiro[3.4]octane-8-carboxylic acid mentioned under target 4-(1-(O-((2-Oxabicyclo[2.2.2]octan-4-yl)methyl)-N—((S)-6-(1-(4-(trifluoromethyl)benzyl)-JH-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid (I-81)

Synthesis of (S)—N-((2S,3R)-1-oxo-1-(2-oxa-7-azaspiro[3.5]nonan-7-yl)-3-((tetrahydro-2H-pyran-4-yl) methoxy) butan-2-yl)-6-(1-(4-(trifluoromethyl) benzyl)-1H-pyrazole-4-carbonyl)-2-(1-(trifluoromethyl)cyclopropane-1-carbonyl)-2,6-diazaspiro[3.4]octane-8-carboxamide (1-67): To a stirred suspension of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (0.300 g, 0.55 mmol) and (2S,3R)-2-amino-1-(2-oxa-7-azaspiro[3.5]nonan-7-yl)-3-((tetrahydro-2H-pyran-4-yl)methoxy)butan-1-one (0.361 g, 1.10 mmol) in DCM (3 mL) were added triethylamine (0.166 g, 1.65 mmol) and T₃P (0.350 g, 1.10 mmol) at room temperature under nitrogen and stirred for 4 h at the same temperature. Reaction mixture was diluted with water (100 mL) and was extracted with dichloromethane (100×2 mL). Combined organic extracts were dried over anhydrous Na₂SO₄ and concentrated under reduce pressure. The crude product was purified by revers-phase column chromatography using acetonitrile: water-−0:1→4:6 as gradient, to afforded (S)—N-((2S,3R)-1-oxo-1-(2-oxa-7-azaspiro[3.5]nonan-7-yl)-3-((tetrahydro-2H-pyran-4-yl) methoxy) butan-2-yl)-6-(1-(4-(trifluoromethyl) benzyl)-1H-pyrazole-4-carbonyl)-2-(1-(trifluoromethyl)cyclopropane-1-carbonyl)-2,6-diazaspiro[3.4]octane-8-carboxamide (0.130 g, 28%) as a white solid. ¹H-NMR (400 MHz, DMSO-d₆) δ 8.46-8.40 (m, 2H), 7.87-7.83 (d, J=16.8 Hz, 1H), 7.73-7.71 (d, J=8.0 Hz, 2H), 7.44-7.43 (d, J=7.6 Hz, 2H), 5.48-5.47 (d, J=6.0 Hz, 2H), 4.86 (br. s, 1H), 4.31-4.12 (m, 6H), 4.12-3.90 (m, 2H), 3.90-3.70 (m, 4H), 3.70-3.50 (m, 3H), 3.50-3.36 (m, 3H), 3.24-3.19 (m, 3H), 2.57-2.54 (m, 2H; merged in DMSO-d₆), 1.82-1.64 (m, 4H), 1.54-1.47 (m, 2H), 1.37-1.04 (m, 7H), 1.03-0.98 (d, J=6.0 Hz, 2H). MS: [MH]⁺ 853.60.

### Synthesis of (8S)—N-((2S,3R)-3-((2-oxabicyclo[2.2.2]octan-4-yl)methoxy)-1-oxo-1-(4-(thiazol-2-yl)piperidin-1-yl)butan-2-yl)-2-((tetrahydrofuran-2-yl)methyl)-6-(thiazole-5-carbonyl)-2,6-diazaspiro[3.4]octane-8-carboxamide (I-44)

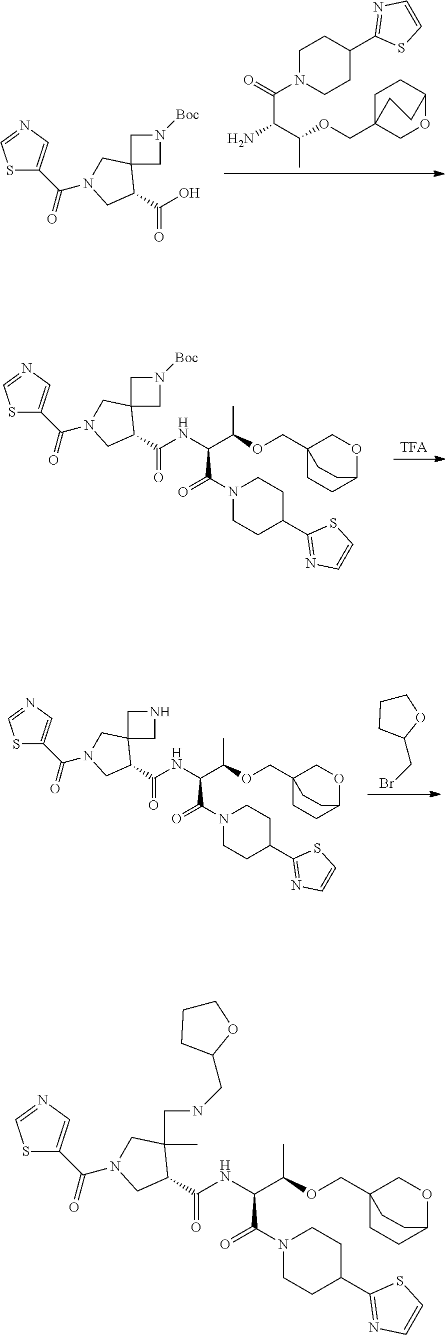

Step 1: tert-butyl (S)-8-(((2S,3R)-3-((2-oxabicyclo[2.2.2]octan-4-yl)methoxy)-1-oxo-1-(4-(thiazol-2-yl)piperidin-1-yl)butan-2-yl)carbamoyl)-6-(thiazole-5-carbonyl)-2,6-diazaspiro[3.4]octane-2-carboxylate: To a solution of (S)-2-(tert-butoxycarbonyl)-6-(thiazole-5-carbonyl)-2,6-diazaspiro[3.4]octane-8-carboxylic acid (206 mg, 0.56 mmol) in DCM (4 mL) was added HATU (213 mg, 0.56 mmol) and DIPEA (217 mg, 1.68 mmol) and the mixture stirred at room temperature for 30 min. (2S,3R)-3-((2-oxabicyclo[2.2.2]octan-4-yl)methoxy)-2-amino-1-(4-(thiazol-2-yl)piperidin-1-yl)butan-1-one (220 mg, 0.56 mmol) was added and the reaction stirred a further 2 h. The reaction was diluted with water (30 mL) and extracted with DCM (50 mL×3). The combined organic layers were washed with brine, dried over Na₂SO₄, filtered and concentrated. The residue obtained was purified by RP-column to afford tert-butyl (S)-8-(((2S,3R)-3-((2-oxabicyclo[2.2.2]octan-4-yl)methoxy)-1-oxo-1-(4-(thiazol-2-yl)piperidin-1-yl)butan-2-yl)carbamoyl)-6-(thiazole-5-carbonyl)-2,6-diazaspiro[3.4]octane-2-carboxylate (170 mg, 45%) as a white solid. LCMS m/z=743.2 [M+H]⁺; ¹H NMR (400 MHz, DMSO-d₆) δ 9.31-9.20 (m, 1H), 8.36 (dd, J=20.2, 12.3 Hz, 2H), 7.76-7.08 (m, 3H), 4.96-4.12 (m, 2H), 4.11-3.70 (m, 10H), 3.67-3.54 (m, 3H), 3.51-3.34 (m, 2H), 3.27-2.70 (m, 2H), 2.25 (s, 1H), 2.14-1.78 (m, 3H), 1.73-1.46 (m, 4H), 1.37 (d, J=5.4 Hz, 9H), 1.34 (s, 3H), 1.24 (s, 1H), 1.17 (t, J=7.1 Hz, 1H), 1.04 (s, 2H).

Step 2: (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-oxo-1-(4-(thiazol-2-yl)piperidin-1-yl)butan-2-yl)carbamoyl)-6-(thiazole-5-carbonyl)-2,6-diazaspiro[3.4]octane-2-carboxylate (85 mg, 0.11 mmol) in DCM (2 mL) was added TFA (1 mL) and the reaction stirred at room temperature for 2 h. The solvent was removed under vacuum to afford (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2,6-diazaspiro[3.4]octane-8-carboxamide (50 mg, 100%) which was used directly in the next step. LCMS m/z=643.2 [M+H]⁺.

Step 3: (8S)—N-((2S,3R)-3-((2-oxabicyclo[2.2.2]octan-4-yl)methoxy)-1-oxo-1-(4-(thiazol-2-yl)piperidin-1-yl)butan-2-yl)-2-((tetrahydrofuran-2-yl)methyl)-6-(thiazole-5-carbonyl)-2,6-diazaspiro[3.4]octane-8-carboxamide (I-44): To a solution of (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2,6-diazaspiro[3.4]octane-8-carboxamide (50 mg, 0.10 mmol) in MeCN (2 mL) was added Na₂CO₃ (27 mg, 0.30 mmol) and 2-(bromomethyl)tetrahydrofuran (18 mg, 0.10 mmol). The mixture was heated at 100° C. in a microwave reactor for 2 h. The mixture was filtered through celite and the filtrate concentrated. The residue obtained was purified by prep-HPLC to afford (8S)—N-((2S,3R)-3-((2-oxabicyclo[2.2.2]octan-4-yl)methoxy)-1-oxo-1-(4-(thiazol-2-yl)piperidin-1-yl)butan-2-yl)-2-((tetrahydrofuran-2-yl)methyl)-6-(thiazole-5-carbonyl)-2,6-diazaspiro[3.4]octane-8-carboxamide (12.4 mg, 19%) as a white solid. LCMS m/z=727.3 [M+H]⁺; ¹H NMR (400 MHz, DMSO-d₆) δ 9.26 (s, 1H), 8.41-8.25 (m, 2H), 8.14 (s, 1H), 7.77-7.54 (m, 2H), 4.87 (s, 1H), 4.41 (s, 1H), 4.26-3.68 (m, 8H), 3.67-3.59 (m, 4H), 3.58-3.53 (m, 2H), 3.25-3.12 (m, 3H), 3.06-3.01 (m, 1H), 2.86-2.72 (m, 2H), 2.18-1.70 (m, 8H), 1.64-1.33 (m, 10H), 1.07 (s, 3H).

### Building Blocks:

### (2S,3R)-3-((2-oxabicyclo[2.2.2]octan-4-yl)methoxy)-2-amino-1-(4-(thiazol-2-yl)piperidin-1-yl)butan-1-one

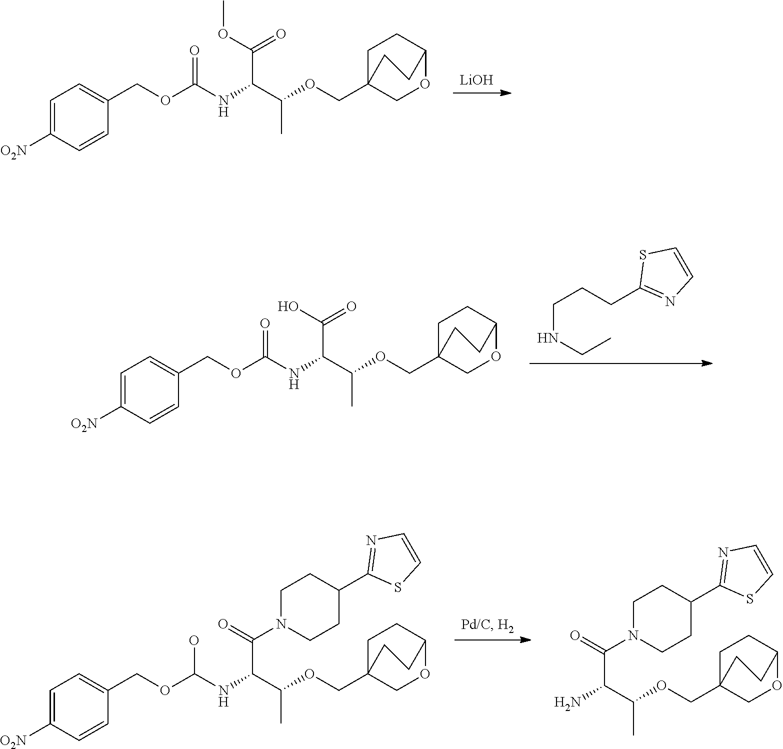

Step 1: O-((2-oxabicyclo[2.2.2]octan-4-yl)methyl)-N-(((4-nitrobenzyl)oxy)carbonyl)-L-threonine: To a solution of methyl O-((2-oxabicyclo[2.2.2]octan-4-yl)methyl)-N-(((4-nitrobenzyl)oxy)carbonyl)-L-threoninate (100 mg, 0.23 mmol) in a mixture of THF, water and MeOH (2.0 mL/0.5 mL/0.5 mL) was added LiOH (24 mg, 0.57 mmol) and the reaction stirred at room temperature for 2 h. The reaction was diluted with water (10 mL) and extracted with ether (30 mL). The aqueous layer was collected and acidified to pH ~2 with 1M HCl then extracted with EtOAc (40 mL×3). The combined organic layers were washed with brine, dried over Na₂SO₄, filtered and concentrated to afford O-((2-oxabicyclo[2.2.2]octan-4-yl)methyl)-N-(((4-nitrobenzyl)oxy)carbonyl)-L-threonine (95 mg, 98%) as a white solid. LCMS m/z=423.1 [M+H]⁺.

Step 2: 4-nitrobenzyl ((2S,3R)-3-((2-oxabicyclo[2.2.2]octan-4-yl)methoxy)-1-oxo-1-(4-(thiazol-2-yl)piperidin-1-yl)butan-2-yl)carbamate: To a solution of O-((2-oxabicyclo[2.2.2]octan-4-yl)methyl)-N-(((4-nitrobenzyl)oxy)carbonyl)-L-threonine (95 mg, 0.23 mmol) in DCM (2 mL) was added HATU (86 mg, 0.23 mmol) and DIPEA (131 mg, 1.02 mmol) and the mixture stirred at room temperature for 30 min. 2-(piperidin-4-yl)thiazole (50 mg, 0.29 mmol) was added and stirring continued for another 2 h. The reaction was diluted with water (10 mL) and extracted with DCM (30 mL×3). The combined organic layers were washed with brine, dried over Na₂SO₄, filtered and concentrated. The residue obtained was purified by prep-TLC (eluent: DCM:MeOH=15:1) and prep-HPLC to afford 4-nitrobenzyl ((2S,3R)-3-((2-oxabicyclo[2.2.2]octan-4-yl)methoxy)-1-oxo-1-(4-(thiazol-2-yl) piperidin-1-yl) butan-2-yl) carbamate (95 mg, 72%) as a white solid. LCMS m/z=573.0 [M+H]⁺; ¹H NMR (400 MHz, DMSO-d₆) δ 8.21 (dd, J=20.3, 8.1 Hz, 2H), 7.72 (s, 1H), 7.67-7.37 (m, 4H), 5.18 (s, 2H), 4.45 (d, J=35.5 Hz, 2H), 4.15 (d, J=29.4 Hz, 1H), 3.65-3.41 (m, 4H), 3.15 (d, J=9.2 Hz, 1H), 2.98 (d, J=9.1 Hz, 1H), 2.86-2.73 (m, 1H), 2.15-1.94 (m, 2H), 1.85-1.64 (m, 2H), 1.50 (s, 6H), 1.41-1.30 (m, 2H), 1.24 (s, 2H), 1.04 (s, 3H).

Step 3: (2S,3R)-3-((2-oxabicyclo[2.2.2]octan-4-yl)methoxy)-2-amino-1-(4-(thiazol-2-yl)piperidin-1-yl)butan-1-one: To a solution of 4-nitrobenzyl ((2S,3R)-3-((2-oxabicyclo[2.2.2]octan-4-yl) methoxy)-1-oxo-1-(4-(thiazol-2-yl) piperidin-1-yl) butan-2-yl) carbamate (95 mg, 0.17 mmol) in MeOH (2 mL) was added 10% Pd/C (38 mg). The reaction was heated at 50° C. under a H₂ atmosphere for 2 h. The catalyst was removed by filtration through celite and the filtrate concentrated to afford (2S,3R)-3-((2-oxabicyclo[2.2.2]octan-4-yl)methoxy)-2-amino-1-(4-(thiazol-2-yl)piperidin-1-yl)butan-1-one (61 mg, 94%) which was used directly in the next step. LCMS m/z=394.3 [M+H]⁺.

### Synthesis of 2-(piperidin-4-yl)thiazole

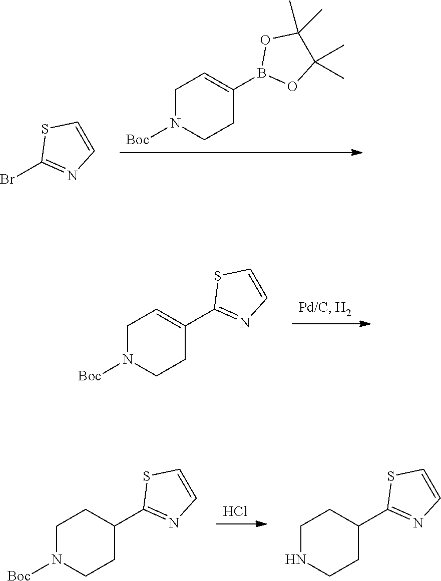

Step 1: tert-butyl 4-(thiazol-2-yl)-3,6-dihydropyridine-1 (2H)-carboxylate: To a solution of 2-bromothiazole (1.0 g, 6.1 mmol) in a mixture of 1,4-dioxane (10 mL) and water (2.5 mL) was added tert-butyl 4-(4,4,5,5-tetramethyl-1,3,2-dioxaborolan-2-yl)-3,6-dihydropyridine-1 (2H)-carboxylate (2.0 g, 6.7 mmol), Na₂CO₃ (1.9 g, 18.3 mmol) and Pd(dppf)Cl₂ (446 mg, 0.61 mol). The reaction was heated at 90° C. under a N₂ atmosphere overnight then was diluted with water (50 mL) and extracted with EtOAc (100 mL×2). The combined organic layers were dried over Na₂SO₄, filtered and concentrated. The residue obtained was purified by column chromatography on silica gel (eluent: Pet. Ether/EtOAc=10/1, v/v) to afford tert-butyl 4-(thiazol-2-yl)-3,6-dihydropyridine-1 (2H)-carboxylate (1.0 g, 63%) as a yellow oil. LCMS m/z=267.1 [M+H]⁺; ¹H NMR (400 MHz, Chloroform-d) δ 7.75 (d, J=3.2 Hz, 1H), 7.22 (d, J=3.3 Hz, 1H), 6.56 (s, 1H), 4.16-4.07 (m, 2H), 3.64 (t, J=5.8 Hz, 2H), 2.70 (s, 2H), 1.48 (s, 9H).

Step 2: tert-butyl 4-(thiazol-2-yl)piperidine-1-carboxylate: To a solution of tert-butyl 4-(thiazol-2-yl)-3,6-dihydropyridine-1 (2H)-carboxylate (430 mg, 1.61 mmol) in MeOH (3 mL) was added 10% Pd/C (215 mg). The reaction was stirred under H₂ atmosphere overnight. The catalyst was removed by filtration through celite and the filtrate concentrated to afford tert-butyl 4-(thiazol-2-yl)piperidine-1-carboxylate (380 mg crude, 88%) as a yellow oil, which was used without purification. LCMS m/z=291.2 [M+Na]⁺.

Step 3: 2-(piperidin-4-yl)thiazole: To a solution of tert-butyl 4-(thiazol-2-yl)piperidine-1-carboxylate (80 mg, 0.30 mmol) in DCM (1 mL) was added a solution of HCl in dioxane (1M, 1 mL). The reaction was stirred at room temperature for 2 h then the solvent was removed under reduced pressure to afford 2-(piperidin-4-yl)thiazole (50 mg, 100%) as a yellow oil, which was used without purification. LCMS m/z=169.2 [M+H]⁺.

### Synthesis of (S)—N-((2S,3R)-3-(2-oxabicyclo[2.2.2]octan-4-ylmethoxy)-1-oxo-1-(4-(thiazol-2-yl)piperidin-1-yl)butan-2-yl)-6-(1-benzyl-1H-pyrazole-4-carbonyl)-2-(1-(trifluoromethyl)cyclopropanecarbonyl)-2,6-diazaspiro[3.4]octane-8-carboxamide (I-69)

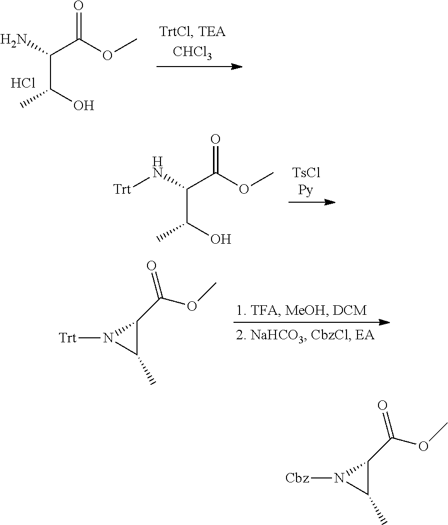

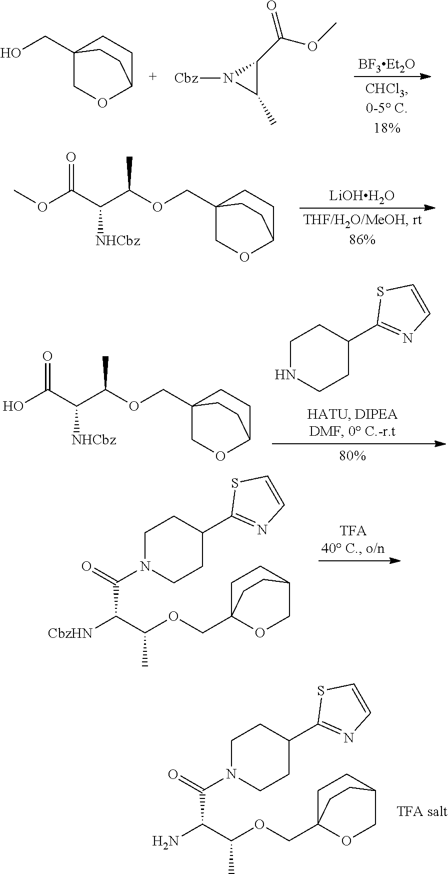

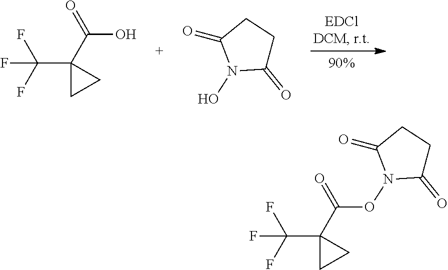 [SMILES: O=C(O)C1(C(F)(F)F)CC1 ; O=C1CCC(=O)N1O ; O=C1CCC(=O)N1OC(=O)C1(C(F)(F)F)CC1]

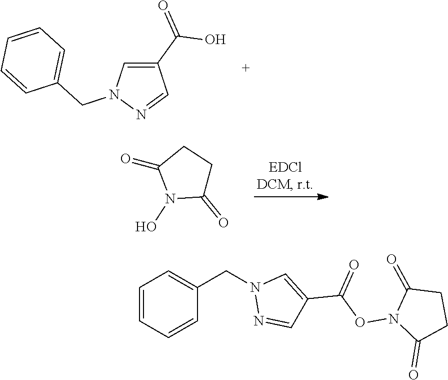 [SMILES: O=C(O)c1cnn(Cc2ccccc2)c1 ; O=C1CCC(=O)N1O ; O=C(ON1C(=O)CCC1=O)c1cnn(Cc2ccccc2)c1]

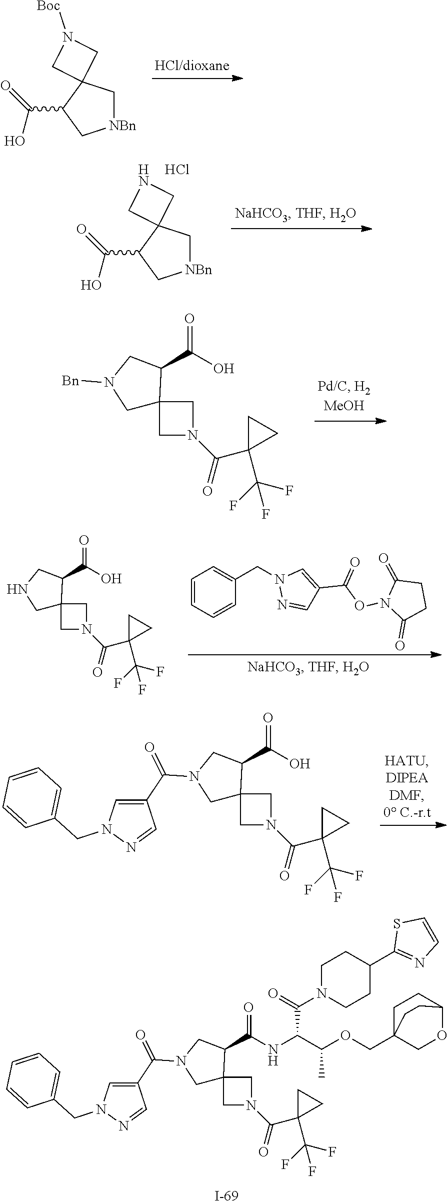

Synthesis of Methyl trityl-L-threoninate: To a solution of methyl L-threoninate hydrochloride (1.00 g, 5.9 mmol) and triethylamine (1.2 g, 11.8 mmol) in chloroform (20 mL) at 0° C. was added TrtCl (1.64 g, 5.9 mmol). The resulting mixture was stirred at 0° C. for 12 hours. The reaction mixture was then diluted with water (30 ml) and extracted with ethyl acetate (10 ml×3). The combined organic phases were washed with brine, dried over anhydrous sodium sulfate, and concentrated under reduced pressure to give a crude residue which was purified by silica gel chromatography using a 2% ethyl acetate in hexane gradient to afford methyl trityl-L-threoninate (1.4 g, 63% yield) as a colorless oil. ¹HNMR (400 MHz, DMSO-d6): δ 7.43 (d, J=7.2 Hz, 6H), 7.27 (t, J=8.0 Hz, 6H), 7.21-7.17 (m, 3H), 5.01 (d, J=4.7 Hz, 1H), 3.90-3.97 (m, 1H), 3.22-3.18 (m, 1H), 3.03 (s, 3H), 2.69 (d, J=10.0 Hz, 1H), 1.09 (d, J=6.4 Hz, 3H).

Synthesis of Methyl (2S,3S)-3-methyl-1-tritylaziridine-2-carboxylate: To a stirred solution of methyl trityl-L-threoninate (10.0 g, 26.6 mmol) in pyridine (30 mL) at 0° C. was added a solution of 4-toluenesulfonyl chloride (15.2 g, 79.9 mmol) in pyridine (40 ml). The resulting mixture was stirred at 0° C. for 20 hours, then raised to 50° C. and stirred for 12 hours. The reaction mixture was concentrated to remove the solvent. The residue was diluted with water (300 ml) and extracted with ethyl acetate (100 ml×3). The combined organic phases were washed with brine, dried over anhydrous sodium sulfate, and concentrated under reduced pressure to give a crude residue which was purified by silica gel chromatography using a 0-2% ethyl acetate in hexane gradient to afford methyl (2S,3S)-3-methyl-1-tritylaziridine-2-carboxylate (6.300 g, 67% yield) as a light yellow oil. ¹HNMR (400 MHz, DMSO-d6): δ 7.42-7.44 (m, 6H), 7.31-7.34 (m, 6H), 7.27-7.24 (m, 3H), 3.67 (s, 3H), 1.72 (d, J=6.4 Hz, 1H), 1.59-1.54 (m, 1H), 1.29 (d, J=5.2 Hz, 3H).

Synthesis of 1-Benzyl 2-methyl (2S,3S)-3-methylaziridine-1,2-dicarboxylate: To a stirred solution of methyl (2S,3S)-3-methyl-1-tritylaziridine-2-carboxylate (5.2 g, 14.7 mmol) in a mixture of methanol (10 mL) and dichloromethane (10 ml) at 0° C. was added 2,2,2-trifluoroacetic acid (11.6 ml, 14.7 mmol). The resulting mixture was stirred at 0° C. for 10 minutes and then concentrated to remove the solvent. The residue was diluted with diethyl ether (30 ml) and extracted with water (20 ml×3). The combined aqueous layer was basified to pH 9 with aqueous sodium bicarbonate solution at 0° C. Ethyl acetate (50 ml) was added to the aqueous solution, followed by addition of benzyl chloroformate (2.5 g, 14.7 mmol) at 0° C. The resulting mixture was stirred at 0° C. for 20 minutes. TLC showed the reaction was complete. The resulting mixture was extracted with ethyl acetate (20 ml×3). The combined organic phase was washed with brine, dried over anhydrous sodium sulfate, and concentrated under reduced pressure to give a crude residue which was purified by silica gel flash chromatography using a 20% ethyl acetate in hexane gradient to afford 1-benzyl 2-methyl (2S,3S)-3-methylaziridine-1,2-dicarboxylate (3.2 g, 89% yield) as a colorless oil. ¹HNMR (400 MHz, DMSO-d6): δ 7.43-7.29 (m, 5H), 5.09 (s, 2H), 3.69 (s, 3H), 3.37 (d, J=6.8 Hz, 1H), 3.00-2.95 (m, 1H), 1.18 (d, J=6.4 Hz, 3H). MS: [MH]⁺ 250.1.

Synthesis of (2S,3R)-methyl 3-(2-oxabicyclo[2.2.2]octan-4-ylmethoxy)-2-(((benzyloxy)carbonyl)amino)butanoate: To a solution of 2-oxabicyclo[2.2.2]octan-4-ylmethanol (0.300 g, 2.110 mmol) and (2S,3S)-1-benzyl 2-methyl 3-methylaziridine-1,2-dicarboxylate (0.526 g, 2.110 mmol) in CHCl₃ (5 mL) at 0° C. was added boron trifluoride-diethyl ether complex (0.060 g, 0.422 mmol) slowly, and the resulting mixture was stirred at 0-5° C. under nitrogen atmosphere for 1 hour. The reaction mixture was concentrated to give a crude residue which was purified by silica gel flash column chromatography using a hexane-ethyl acetate-dichloromethane (7:1:1 v/v/v) gradient then a hexane-ethyl acetate-dichloromethane (3:1:1 v/v/v) gradient to afford (2S,3R)-methyl 3-(2-oxabicyclo[2.2.2]octan-4-ylmethoxy)-2-(((benzyloxy)carbonyl)amino)butanoate (0150 g, 18%) as a colorless oil. MS: [MH]⁺392.20.

Synthesis of (2S,3R)-3-(2-oxabicyclo[2.2.2]octan-4-ylmethoxy)-2-(((benzyloxy)carbonyl)amino)butanoic acid: To a solution of (2S,3R)-methyl 3-(2-oxabicyclo[2.2.2]octan-4-ylmethoxy)-2-(((benzyloxy)carbonyl)amino)butanoate (0.660 g, 1.686 mmol) in tetrahydrofuran (8 mL)-methanol (4 mL)-water (2 mL) was added lithium hydroxide monohydrate (0.142 g, 3.372 mmol); the resulting mixture was stirred at room temperature for 3 hours, and then concentrated. The residue was diluted with water (10 mL) and extracted with ether (10 mL). The aqueous layer was acidified to pH 3-4 with hydrochloric acid (2.0 N), and extracted with dichloromethane (15 mL×3). The combined organic layers were dried over anhydrous sodium sulfate and concentrated to give (2S,3R)-3-(2-oxabicyclo[2.2.2]octan-4-ylmethoxy)-2-(((benzyloxy)carbonyl)amino)butanoic acid (0.550 g, yield 86%) as a colorless oil which was used in the next step without further purification. MS: [MH]⁺ 378.20

Synthesis of Benzyl ((2S,3R)-3-(2-oxabicyclo[2.2.2]octan-4-ylmethoxy)-1-oxo-1-(4-(thiazol-2-yl)piperidin-1-yl)butan-2-yl)carbamate: To a solution of (2S,3R)-3-(2-oxabicyclo[2.2.2]octan-4-ylmethoxy)-2-(((benzyloxy)carbonyl)amino)butanoic acid (0.570 g, 1.510 mmol), 2-(piperidin-4-yl)thiazole (0.280 g, 1.661 mmol), and N-ethyl-N-isopropylpropan-2-amine (0.585 g, 4.530 mmol) in N,N-dimethylformamide (11 mL) at 0-5° C. was added (2-(7-Aza-1H-benzotriazole-1-yl)-1,1,3,3-tetramethyluronium hexafluorophosphate) (HATU, 0.689 g, 1.812 mmol). The resulting mixture was stirred at room temperature for 1 hour. The reaction mixture was poured into water (20 mL) and extracted with ethyl acetate (10 mL). The organic phase was washed with water (10 mL×3) and brine (10 mL), dried over anhydrous sodium sulfate, and concentrated under reduced pressure to give a crude residue which was purified by silica gel flash column chromatography using a 10% ethyl acetate/hexane gradient to afford benzyl ((2S,3R)-3-(2-oxabicyclo[2.2.2]octan-4-ylmethoxy)-1-oxo-1-(4-(thiazol-2-yl)piperidin-1-yl)butan-2-yl)carbamate (0.680 g, 80% yield) as a colorless oil. MS: [MH]⁺ 528.60.

Synthesis of (2S,3R)-3-(2-oxabicyclo[2.2.2]octan-4-ylmethoxy)-2-amino-1-(4-(thiazol-2-yl)piperidin-1-yl)butan-1-one TFA salt: A solution of benzyl ((2S,3R)-3-(2-oxabicyclo[2.2.2]octan-4-ylmethoxy)-1-oxo-1-(4-(thiazol-2-yl)piperidin-1-yl)butan-2-yl)carbamate (0.600 g, 1.136 mmol) in trifluoroacetic acid (6 mL) was stirred at 40° C. overnight. The reaction mixture was concentrated to give (2S,3R)-3-(2-oxabicyclo[2.2.2]octan-4-ylmethoxy)-2-amino-1-(4-(thiazol-2-yl)piperidin-1-yl)butan-1-one TFA salt (575 mg, 100% yield) as a yellow oil which was used in the next step without further purification. MS: [MH]⁻ 395.10.

Synthesis of 2,5-Dioxopyrrolidin-1-yl 1-(trifluoromethyl)cyclopropane-1-carboxylate: A mixture of 1-(trifluoromethyl)cyclopropane-1-carboxylic acid (20.000 g, 129.87 mmol), 1-hydroxypyrrolidine-2,5-dione (14.930 g, 129.87 mmol), and EDCI (30.000 g, 156.25 mmol) in dichloromethane (100 mL) was stirred at room temperature under nitrogen atmosphere overnight. The reaction mixture was concentrated to give a crude residue which was purified by silica gel column chromatography using a dichloromethane gradient to afford 2,5-dioxopyrrolidin-1-yl 1-(trifluoromethyl)cyclopropane-1-carboxylate (29.500 g, yield 90%) as a white solid. ¹HNMR (400 MHz, CDCl₃): δ 2.83 (s, 411), 1.78-1.75 (m, 2H), 1.61-1.58 (m, 2H).

Synthesis of 2,5-Dioxopyrrolidin-1-yl 1-benzyl-1H-pyrazole-4-carboxylate: A mixture of 1-benzyl-1H-pyrazole-4-carboxylic acid (10.000 g, 49.50 mmol), 1-hydroxypyrrolidine-2,5-dione (14.930 g, 129.87 mmol), and EDCI (9.500 g, 49.50 mmol) in dichloromethane (70 mL) was stirred at room temperature under nitrogen atmosphere overnight. The reaction mixture was concentrated to give a crude residue which was purified by silica gel flash column chromatography using a dichloromethane gradient to afford 2,5-dioxopyrrolidin-1-yl 1-benzyl-1H-pyrazole-4-carboxylate (11.840 g, yield 80%) as a white solid. MS: [MH]⁺) 300.20

Synthesis of (S)-6-benzyl-2,6-diazaspiro[3.4]octane-8-carboxylic acid hydrochloride: To a mixture of (S)-6-benzyl-2-(tert-butoxycarbonyl)-2,6-diazaspiro[3.4]octane-8-carboxylic acid (5.000 g, 14.43 mmol) in dichloromethane (20 mL) was added hydrogen chloride in 1,4-dioxane (4.0 M, 10 mL); the resulting mixture was stirred at room temperature under nitrogen atmosphere overnight. TLC showed the reaction was complete. The reaction mixture was concentrated under reduced pressure to afford (S)-6-benzyl-2,6-diazaspiro[3.4]octane-8-carboxylic acid hydrochloride (4.090 g, 100% yield) as a white solid which was used in next step without feather purification. MS: [MH]⁺ 247.40.

Synthesis of (S)-6-benzyl-2-(1-(trifluoromethyl)cyclopropanecarbonyl)-2,6-diazaspiro[3.4]octane-8-carboxylic acid: To a solution of (S)-6-benzyl-2,6-diazaspiro[3.4]octane-8-carboxylic acid hydrochloride (4.090 g, 14.43 mmol) in water (40 mL) was added sodium bicarbonate (6.100 g, 72.17 mmol) slowly, followed by addition of a solution of 2,5-dioxopyrrolidin-1-yl 1-(trifluoromethyl)cyclopropanecarboxylate (3.600 g, 14.43 mmol) in tetrahydrofuran (40 mL). The resulting mixture was stirred at room temperature for 4 hours. The reaction mixture was adjusted pH 6-7 with hydrochloric acid (2.0N), and extracted with a mixture of 25% isopropyl alcohol in dichloromethane (40 mL×3). The combined organic layers were dried over anhydrous sodium sulfate, and concentrated to give a crude residue which was purified by silica gel column chromatography using a 10-30% methanol in dichloromethane gradient to afford (S)-6-benzyl-2-(1-(trifluoromethyl)cyclopropanecarbonyl)-2,6-diazaspiro[3.4]octane-8-carboxylic acid (4.500 g, 81% yield) as a white solid. MS: [MH]⁺ 383.80.

Synthesis of (S)-2-(1-(trifluoromethyl)cyclopropane-1-carbonyl)-2,6-diazaspiro[3.4]octane-8-carboxylic acid: A mixture of (S)-6-benzyl-2-(1-(trifluoromethyl)cyclopropanecarbonyl)-2,6-diazaspiro[3.4]octane-8-carboxylic acid (4.500 g, 11.75 mmol) and Pd/C (10%, 0.400 g) in methanol (50 mL) was stirred under hydrogen atmosphere at room temperature overnight. The reaction mixture was filtered to remove the catalyst, and the filtrate was concentrated to give a crude residue which was purified by silica gel column chromatography using a 10% methanol in dichloromethane gradient to afford (S)-2-(1-(trifluoromethyl)cyclopropane-1-carbonyl)-2,6-diazaspiro[3.4]octane-8-carboxylic acid (2.600 g, 76%) as a white solid. ¹HNMR (400 MHz, CDCl₃): δ 4.40-3.93 (m, 4H), 3.24-3.19 (m, 3H), 3.13 (d, J=10.8 Hz, 1H), 2.89 (t, J=6.8 Hz, 1H), 1.22 (s, 4H).

Synthesis of (S)-6-(1-benzyl-1H-pyrazole-4-carbonyl)-2-(1-(trifluoromethyl)cyclopropanecarbonyl)-2,6-diazaspiro[3.4]octane-8-carboxylic acid: To a mixture of (S)-2-(1-(trifluoromethyl)cyclopropane-1-carbonyl)-2,6-diazaspiro[3.4]octane-8-carboxylic acid (1.000 g, 3.42 mmol) and sodium bicarbonate (0.863 g, 10.29 mmol) in water (10 mL) was added a solution of 2,5-dioxopyrrolidin-1-yl 1-benzyl-1H-pyrazole-4-carboxylate (1.022 g, 3.42 mmol) in tetrahydrofuran (10 mL), and the resulting mixture was stirred at room temperature for 4 hours. The reaction mixture was adjusted pH 6-7 with hydrochloric acid (2.0 N), and extracted with a mixture of 25% isopropyl alcohol in dichloromethane (10 mL×3). The combined organic layers were dried over anhydrous sodium sulfate, and concentrated to give a crude residue which was purified by silica gel column chromatography using a 10% methanol in dichloromethane gradient to afford (S)-6-(1-benzyl-1H-pyrazole-4-carbonyl)-2-(1-(trifluoromethyl)cyclopropanecarbonyl)-2,6-diazaspiro[3.4]octane-8-carboxylic acid (1.140 g, 70% yield) as a white solid. MS: [MH]⁺ 477.30.

Synthesis of (S)—N-((2S,3R)-3-(2-oxabicyclo[2.2.2]octan-4-ylmethoxy)-1-oxo-1-(4-(thiazol-2-yl)piperidin-1-yl)butan-2-yl)-6-(1-benzyl-1H-pyrazole-4-carbonyl)-2-(1-(trifluoromethyl)cyclopropanecarbonyl)-2,6-diazaspiro[3.4]octane-8-carboxamide (1-69): To a solution of (2S,3R)-3-(2-oxabicyclo[2.2.2]octan-4-ylmethoxy)-2-amino-1-(4-(thiazol-2-yl)piperidin-1-yl)butan-1-one TFA salt (0.064 g, 0.130 mmol), (S)-6-(1-benzyl-1H-pyrazole-4-carbonyl)-2-(1-(trifluoromethyl)cyclopropanecarbonyl)-2,6-diazaspiro[3.4]octane-8-carboxylic acid (0.060 g, 0.125 mmol), and N-ethyl-N-isopropylpropan-2-amine (1.2 mL) in N,N-dimethylformamide (5 mL) at 0-5° C. was added (2-(7-Aza-1H-benzotriazole-1-yl)-1,1,3,3-tetramethyluronium hexafluorophosphate) (0.052 g, 0.137 mmol). The resulting mixture was stirred at room temperature for 2 hours. The reaction mixture was poured into water (15 mL) and extracted with ethyl acetate (10 mL×2). The combined organic layer was washed with water (10 mL×3) and brine (10 mL), dried over anhydrous sodium sulfate, and concentrated under reduced pressure to give a crude residue which was purified by prep-TLC using a 7% methanol in dichloromethane gradient to afford (S)—N-((2S,3R)-3-(2-oxabicyclo[2.2.2]octan-4-ylmethoxy)-1-oxo-1-(4-(thiazol-2-yl)piperidin-1-yl)butan-2-yl)-6-(1-benzyl-1H-pyrazole-4-carbonyl)-2-(1-(trifluoromethyl)cyclopropanecarbonyl)-2,6-diazaspiro[3.4]octane-8-carboxamide I-69 (0.052 g, 53% yield) as a white solid. ¹H NMR (400 MHz, CDCl₃) δ8.22-8.12 (m, 2H), 7.93-7.90 (m, 1H), 7.71-7.66 (m, 1H), 7.48-7.27 (m, 5H), 5.37 (s, 2H), 7.98-7.94 (m, 1H), 4.57-3.82 (m, 10H), 3.70 (s, 4H), 3.48-3.34 (m, 3H), 3.27-3.22 (m, 1H), 3.05-3.03 (m, 1H), 2.90-2.83 (m, 1H), 2.22-2.14 (m, 2H), 2.01-1.97 (m, 2H), 1.82-1.79 (m, 1H), 1.64-1.61 (m, 5H), 1.52-1.49 (m, 2H), 1.23 (s, 4H), 1.16-1.12 (m, 3H). MS: [MH]⁺ 852.55.

The following 6 compounds were prepared in a manner analogous to the procedures described above for (S)—N-((2S,3R)-3-(2-oxabicyclo[2.2.2]octan-4-ylmethoxy)-1-oxo-1-(4-(thiazol-2-yl)piperidin-1-yl)butan-2-yl)-6-(1-benzyl-1H-pyrazole-4-carbonyl)-2-(1-(trifluoromethyl)cyclopropanecarbonyl)-2,6-diazaspiro[3.4]octane-8-carboxamide (I-69):

Synthesis of (S)—N-((2S,3R)-3-(2-oxabicyclo[2.2.2]octan-4-ylmethoxy)-1-oxo-1-(4-(thiazol-2-yl)piperidin-1-yl)butan-2-yl)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamide (I-91) (0.050 g, 28%) as a white solid. ¹HNMR (400 MHz, CD₃OD): δ 8.30-8.25 (m, 1H), 7.96-7.93 (m, 1H), 7.72-7.64 (m, 3H), 7.49-7.40 (m, 3H), 5.48 (s, 2H), 4.98-4.93 (m, 1H), 4.62-4.52 (m, 1H), 4.37-3.98 (m, 7H), 3.87-3.81 (m, 1H), 3.75-3.70 (m, 4H), 3.47-3.34 (m, 3H), 3.27-3.22 (m, 1H), 3.06-3.01 (m, 1H), 2.90-2.83 (m, 1H), 2.23-2.14 (m, 2H), 2.00-1.96 (m, 2H), 1.82-1.64 (m, 6H), 1.52-1.47 (m, 2H), 1.23-1.19 (m, 4H), 1.16-1.13 (m, 3H). MS: [MH]⁺ 920.80.

Synthesis of ((4-((S)-8-(((2S,3R)-3-((2-oxabicyclo[2.2.2]octan-4-yl)methoxy)-1-oxo-1-(4-(thiazol-2-yl)piperidin-1-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)-5-(trifluoromethyl)benzoic acid (I-83) (0.024 g, yield 37%) as a gray solid. ¹HNMR (400 MHz, CD₃OD): δ 8.31-8.28 (m, 1H), 8.07 (s, 1H), 7.93 (d, J=6.0 Hz, 1H), 7.71-7.42 (m, 3H), 7.16-6.98 (m, 1H), 5.83 (s, 2H), 5.00-4.83 (m, 1H), 4.68-3.63 (m, 14H), 3.45-2.79 (m, 5H), 2.24-1.83 (m, 5H), 1.71-1.44 (m, 7H), 1.23-1.12 (m, 8H). MS: [MH]⁺ 964.80

Synthesis of 2-((4-((S)-8-(((2S,3R)-3-(2-oxabicyclo[2.2.2]octan-4-ylmethoxy)-1-oxo-1-(4-(thiazol-2-yl)piperidin-1-yl)butan-2-yl)carbamoyl)-2-(1-(trifluoromethyl)cyclopropanecarbonyl)-2,6-diazaspiro[3.4]octane-6-carbonyl)-1H-pyrazol-1-yl)methyl)benzoic acid (I-88) (0.025 g, yield 65%) as a white solid. ¹H NMR (400 MHz, CD₃OD) δ 8.24-8.20 (m, 1H), 8.01 (d, J=3.8 Hz, 1H), 7.94-7.91 (m, 1H), 7.71-7.68 (m, 1H), 7.50-7.39 (m, 3H), 7.00-6.96 (m, 1H), 5.81 (s, 2H), 4.98-4.94 (m, 1H), 4.57-3.83 (m, 10H), 3.70 (s, 4H), 3.45-3.37 (m, 2H), 3.27-3.22 (m, 1H), 3.05-3.03 (m, 1H), 2.90-2.83 (m, 1H), 2.19-2.14 (m, 2H), 2.03-1.93 (m, 2H), 1.82-1.79 (m, 1H), 1.64-1.61 (m, 5H), 1.52-1.49 (m, 2H), 1.23-1.22 (m, 4H), 1.16-1.12 (m, 3H), 0.91-0.88 (m, 1H). MS: [MH]⁺ 896.40.

Synthesis of 4-(4-(O-((2-oxabicyclo[2.2.2]octan-4-yl)methyl)-N—((S)-6-(1-benzyl-1H-pyrazole-4-carbonyl)-2-(1-(trifluoromethyl)cyclopropane-1-carbonyl)-2,6-diazaspiro[3.4]octane-8-carbonyl)-L-threonyl)piperazin-1-yl)benzoic acid (I-87) (18 mg, yield 47%) as white solid. ¹H NMR (400 MHz, MeOD) δ 8.23-8.13 (m, 1H), 7.91-7.87 (m, 3H), 7.34-7.26 (m, 5H), 6.99-6.95 (m, 2H), 5.37-5.34 (m, 2H), 4.95-4.94 (m, 1H), 4.57-4.34 (m, 2H), 4.21-3.99 (m, 5H), 3.87-3.66 (m, 10H), 3.44-3.36 (m, 4H), 3.26-3.23 (m, 1H), 3.06-2.98 (m, 1H), 2.02-1.93 (m, 2H), 1.63-1.61 (m, 4H), 1.51-1.48 (m, 2H), 1.23-1.49 (m, 6H). MS: [MH]⁺ 890.80

Synthesis of (S)—N-((2S,3R)-3-((2-oxabicyclo[2.2.2]octan-4-yl)methoxy)-1-oxo-1-(4-(1-oxoisochroman-6-yl)piperidin-1-yl)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (1-66) (0.150 g, yield 42%) as a white solid. ¹HNMR (400 MHz, CD₃OD): δ 8.30-8.25 (m, 1H), 7.97-7.87 (m, 2H), 7.66-7.61 (m, 2H), 7.46-7.39 (m, 2H), 7.34-7.23 (m, 2H), 5.49-5.47 (m, 2H), 5.03-4.92 (m, 2H), 4.72-3.08 (m, 10H), 3.73-3.62 (m, 4H), 3.53-2.72 (m, 8H), 2.03-1.46 (m, 11H), 1.26-1.13 (m, 8H). MS: [MH]⁺983.35.

Synthesis of 4-(4-((2R,3R)-3-(2-oxabicyclo[2.2.2]octan-4-ylmethoxy)-2-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butoxy)cyclohexyl)benzoic acid (I-86) (0.065 g, yield 55%) as a white solid. ¹HNMR (400 MHz, CD₃OD) δ8.30-8.26 (m, 1H), 8.04-7.86 (m, 4H), 7.66-7.62 (m, 2H), 7.45-7.40 (m, 2H), 7.33-7.30 (m, 2H), 5.48-5.45 (m, 2H), 4.58-4.34 (m, 2H), 4.24-4.22 (m, 1H), 4.07-3.96 (m, 4H), 3.84-3.71 (m, 5H), 3.64-3.49 (m, 3H), 3.37-3.35 (m, 2H), 3.27-3.24 (m, 1H), 3.02-2.96 (m, 1H), 2.60-2.49 (m, 1H), 2.17-1.89 (m, 6H), 1.73-1.50 (m, 8H), 1.40-1.34 (m, 1H), 1.24-1.23 (m, 5H), 1.10-1.05 (m, 3H). MS: [MH]⁺ 958.45.

### Synthesis of 1-((((2R,3S)-4-oxo-4-(4-(thiazol-2-yl)piperidin-1-yl)-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylic acid (I-76)

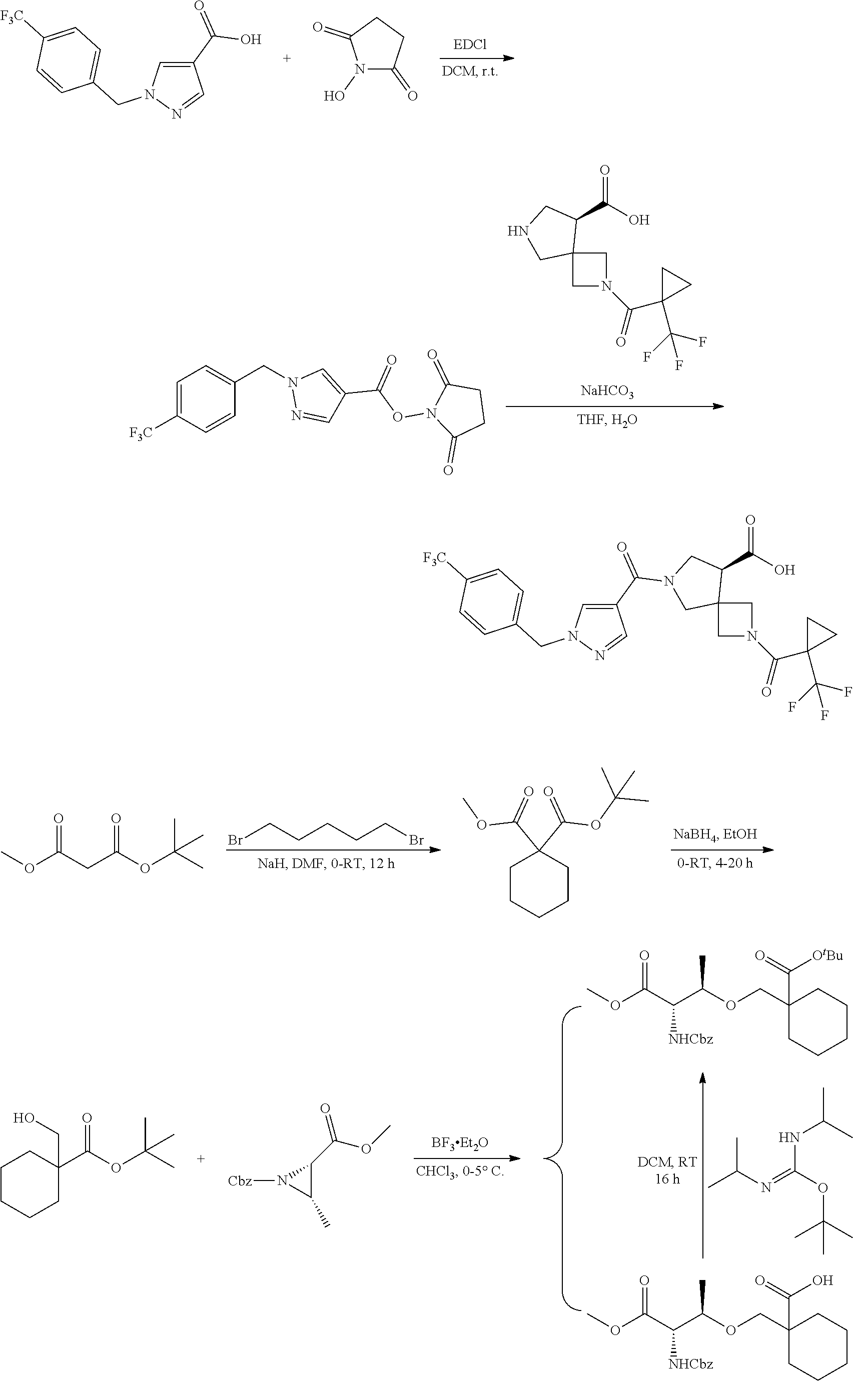

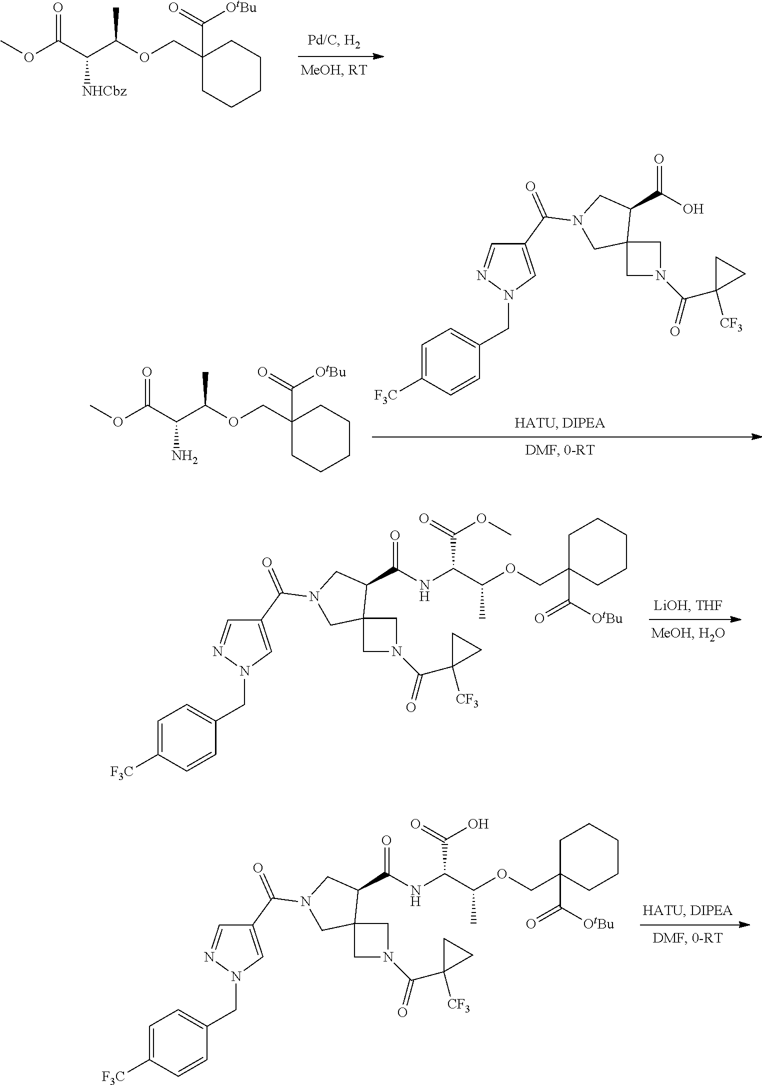 [SMILES: COC(=O)[C@@H](NC(=O)OCc1ccccc1)[C@@H](C)OCC1(C(=O)OC(C)(C)C)CCCCC1 ; COC(=O)[C@@H](N)[C@@H](C)OCC1(C(=O)OC(C)(C)C)CCCCC1 ; O=C(O)[C@@H]1CN(C(=O)c2cnn(Cc3ccc(C(F)(F)F)cc3)c2)CC12CN(C(=O)C1(C(F)(F)F)CC1)C2 ; COC(=O)[C@@H](NC(=O)[C@@H]1CN(C(=O)c2cnn(Cc3ccc(C(F)(F)F)cc3)c2)CC12CN(C(=O)C1(C(F)(F)F)CC1)C2)[C@@H](C)OCC1(C(=O)OC(C)(C)C)CCCCC1 ; C[C@@H](OCC1(C(=O)OC(C)(C)C)CCCCC1)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)O]

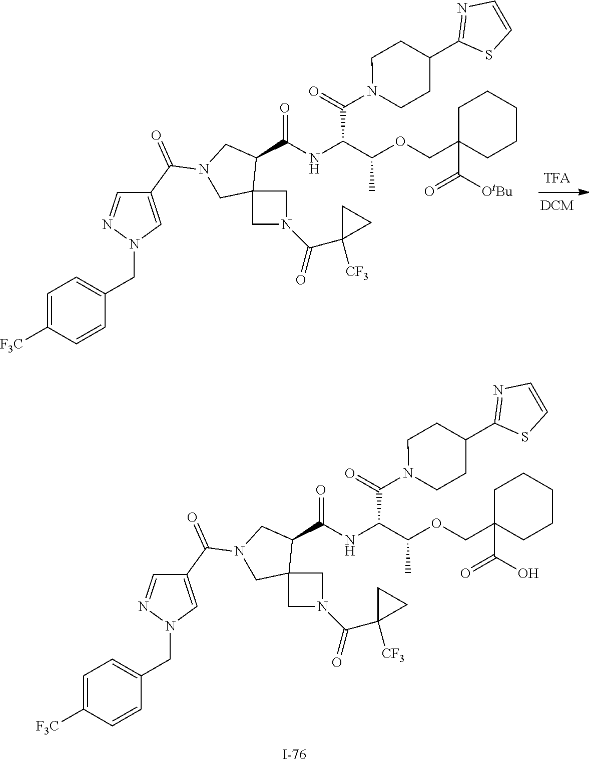

Synthesis of 2,5-Dioxopyrrolidin-1-yl 1-(4-(trifluoromethyl)benzyl)-1H-pyrazole-4-carboxylate: A mixture of 1-(4-(trifluoromethyl)benzyl)-1H-pyrazole-4-carboxylic acid (10.000 g, 37.04 mmol), 1-hydroxypyrrolidine-2,5-dione (10.650 g, 92.60 mmol), and EDCI (7.110 g, 37.04 mmol) in dichloromethane (70 mL) was stirred at room temperature under nitrogen atmosphere overnight. The reaction mixture was concentrated to give a crude residue which was purified by silica gel flash column chromatography using a dichloromethane gradient to afford 2,5-dioxopyrrolidin-1-yl 1-(4-(trifluoromethyl)benzyl)-1H-pyrazole-4-carboxylate (10.87 g, yield 80%) as a white solid. MS: [MH]⁺ 368.20

Synthesis of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid: To a mixture of (S)-2-(1-(trifluoromethyl)cyclopropane-1-carbonyl)-2,6-diazaspiro[3.4]octane-8-carboxylic acid (1.000 g, 3.42 mmol) and sodium bicarbonate (0.863 g, 10.29 mmol) in water (10 mL) was added 2,5-dioxopyrrolidin-1-yl 1-benzyl-1H-pyrazole-4-carboxylate (1.255 g, 3.42 mmol) in tetrahydrofuran (10 mL), and the resulting mixture was stirred at room temperature for 4 hours. The reaction mixture was adjusted pH 6-7 with hydrochloric acid (2.0N) and extracted with a mixture of isopropyl alcohol in dichloromethane (3:1, v/v, 10 mL×3). The combined organic layers were dried over anhydrous sodium sulfate and concentrated to give a crude residue which was purified by silica gel flash column chromatography using a 10% methanol in dichloromethane gradient to afford (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (1.395 g, 75% yield) as a white solid. MS: [MH]⁺ 545.30.

Synthesis of Tert-butyl 1-methyl cyclohexane-1,1-dicarboxylate: To a suspension of sodium hydride (60% in mineral oil, 6.900 g, 172.41 mmol) in N,N-dimethylacetamide (150 mL) at 0-5° C. under nitrogen atmosphere was added a solution of tert-butyl methyl malonate (10.00 g, 57.47 mmol) in N,N-dimethylacetamide (45 mL). The resulting mixture was stirred at 0-5° C. for 0.5 hour, followed by addition of 1,5-dibromopentane (17.18 g, 74.71 mmol) at 0-5° C. The resulting mixture was stirred at room temperature under nitrogen atmosphere for 15 hours. The reaction mixture was poured into ice water (800 mL), extracted with ethyl acetate (500 mL), washed with water (800 mL×2) and brine (300 mL), dried over anhydrous sodium sulfate, and concentrated under reduced pressure to give a crude which was purified by silica gel flash column chromatography using a 3% ethyl acetate in hexane gradient to afford 1-tert-butyl 1-methyl cyclohexane-1,1-dicarboxylate (5.900 g, 42% yield) as a colorless oil. ¹HNMR (400 MHz, CDCl₃): δ 3.70 (s, 3H), 1.94-1.91 (m, 4H), 1.53-1.49 (m, 4H), 1.43-1.42 (m, 11H).

Synthesis of Tert-butyl 1-(hydroxymethyl)cyclohexanecarboxylate: To a solution of 1-tert-butyl 1-methyl cyclohexane-1,1-dicarboxylate (5.800 g, 23.97 mmol) in ethanol (80 mL) at 0-5° C. was added sodium borohydride (36.400 g, 95.87 mmol). The resulting mixture was stirred at room temperature for 15 hours, quenched with ice water (100 mL), and extracted with ethyl acetate (100 mL×2). The combined organic layers were washed with brine (150 mL), dried over anhydrous sodium sulfate, and concentrated under reduced pressure to give a crude residue which was purified by silica gel column chromatography using a 10% ethyl acetate in hexane to afford tert-butyl 1-(hydroxymethyl)cyclohexanecarboxylate (2.700 g, 49% yield) as a colorless oil. ¹HNMR (400 MHz, CDCl₃): δ 3.56 (d, J=6.4 Hz, 2H), 2.11 (d, J=6.4 Hz, 1H), 1.96-1.91 (m, 2H), 1.57-1.53 (m, 3H), 1.47-1.40 (m, 1H), 1.33-1.26 (m, 3H).

Synthesis of Tert-butyl 1-((((2R,3S)-3-(((benzyloxy)carbonyl)amino)-4-methoxy-4-oxobutan-2-yl)oxy)methyl)cyclohexanecarboxylate: To a mixture of tert-butyl 1-(hydroxymethyl)cyclohexanecarboxylate (1.100 g, 5.14 mmol) and (2S,3S)-1-benzyl 2-methyl 3-methylaziridine-1,2-dicarboxylate (1.28 g, 5.14 mmol) in chloroform (25 ml) at 0-5° C. under nitrogen atmosphere was added boron trifluoride diethyl etherate (0.3 mL, 1.02 mmol). The resulting mixture was stirred at room temperature nitrogen atmosphere for 2 hours. The reaction mixture was concentrated under reduced pressure to give a crude residue which was purified by silica gel flash column chromatography using a 20% ethyl acetate in hexane gradient to afford 1-((((2R,3 S)-3-(((benzyloxy)carbonyl)amino)-4-methoxy-4-oxobutan-2-yl)oxy)methyl)cyclohexanecarboxylic acid (0.800 g, 38%) as a colorless oil. MS: [MH]⁺ 408.20.

To a solution of 1-((((2R,3S)-3-(((benzyloxy)carbonyl)amino)-4-methoxy-4-oxobutan-2-yl)oxy)methyl)cyclohexanecarboxylic acid (770.0 mg, 1.89 mmol) in dichloromethane (10 mL) was added 2-tert-butyl-1,3-diisopropyl-isourea (1.130 g, 5.66 mmol) at room temperature. The mixture was stirred at room temperature for 1 hour, followed by additional of second portion of 2-tert-Butyl-1,3-diisopropyl-isourea (566.8 mg, 2.83 mmol). The resulting mixture was stirred at room temperature under nitrogen atmosphere for 30 hours. The reaction mixture was concentrated under reduced pressure to give a crude residue which was purified by silica gel column chromatography using a 10% ethyl acetate in hexane gradient to afford tert-butyl 1-((((2R,3S)-3-(((benzyloxy)carbonyl)amino)-4-methoxy-4-oxobutan-2-yl)oxy)methyl)cyclohexanecarboxylate (0.520 g, 59% yield) as a colorless oil. MS: [MH]⁺ 464.05.

Synthesis of Tert-butyl 1-((((2R,3S)-3-amino-4-methoxy-4-oxobutan-2-yl)oxy)methyl)cyclohexanecarboxylate: A mixture of tert-butyl 1-((((2R,3S)-3-(((benzyloxy)carbonyl)amino)-4-methoxy-4-oxobutan-2-yl)oxy)methyl)cyclohexanecarboxylate (0.500 g, 1.08 mmol) and palladium on carbon (10%, 60.0 mg) in methanol (25 mL) was stirred at 35° C. under hydrogen atmosphere (hydrogen balloon) overnight. Palladium on carbon was removed through filtration and washed with methanol (20 ml×2). The combined filtrates were concentrated under reduced pressure to give crude tert-butyl 1-((((2R,3S)-3-amino-4-methoxy-4-oxobutan-2-yl)oxy)methyl)cyclohexanecarboxylate (320.0 mg, 92%) as colorless oil which was used in next step without further purification.

Synthesis of Tert-butyl 1-((((2R,3S)-4-methoxy-4-oxo-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylate: To a mixture of tert-butyl 1-((((2R,3S)-3-amino-4-methoxy-4-oxobutan-2-yl)oxy)methyl)cyclohexanecarboxylate (0.320 g, 0.97 mmol), (S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxylic acid (0.527 g, 0.97 mmol), and N-ethyl-N-isopropylpropan-2-amine (375.4 mg, 2.91 mmol) in N,N-dimethylformamide (5 mL) at 0-5° C. was added HATU (2-(7-Aza-1H-benzotriazole-1-yl)-1,1,3,3-tetramethyluronium hexafluorophosphate) (0.553 g, 1.46 mmol). The resulting mixture was stirred at room temperature for 2 hours. The reaction mixture was poured into water (30 mL) and extracted with ethyl acetate (15 mL×3). The combined organic layers were washed with water (35 mL) and brine (30 mL), dried over anhydrous sodium sulfate, and concentrated under reduced pressure to give a crude residue which was purified by silica gel flash column chromatography using a 5% methanol in dichloromethane gradient to afford tert-butyl 1-((((2R,3S)-4-methoxy-4-oxo-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylate (0.460 g, 55% yield) as an off white solid. MS: [MH]⁺ 856.45.

Synthesis of (2S,3R)-3-((1-(tert-butoxycarbonyl)cyclohexyl)methoxy)-2-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butanoic acid A solution of tert-butyl 1-((((2R,3S)-4-methoxy-4-oxo-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylate (0.460 g, 0.54 mmol) and lithium hydroxide monohydrate (0.045 g, 1.08 mmol) in and mixture of tetrahydrofuran-methanol-water (1.5 mL-0.5 mL-0.5 mL) was stirred at room temperature for 2 hours. The reaction mixture was adjusted pH to 5-6 with hydrochloric acid (0.5M) at 0-5° C., and extracted with ethyl acetate (15 mL×3). The combined organic layers were washed with water (25 mL) and brine (20 mL), dried over anhydrous sodium sulfate, and concentrated under reduced pressure to give a crude residue which was purified by silica gel column chromatography using a 5% methanol in dichloromethane gradient to afford (2S,3R)-3-((1-(tert-butoxycarbonyl)cyclohexyl)methoxy)-2-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butanoic acid (0.380 g, 83%) as a white solid. MS: [MH]⁺ 842.15.

Synthesis of Tert-butyl 1-((((2R,3S)-4-oxo-4-(4-(thiazol-2-yl)piperidin-1-yl)-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylate: To a mixture of (2S,3R)-3-((1-(tert-butoxycarbonyl)cyclohexyl)methoxy)-2—((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butanoic acid (0.180 g, 0.21 mmol), 2-(piperidin-4-yl)thiazole (0,036 g, 0.21 mmol), and N-ethyl-N-isopropylpropan-2-amine (0.081 g, 0.63 mmol) in N,N-dimethylformamide (5 mL) at 0-5° C. was added HATU (2-(7-Aza-1H-benzotriazole-1-yl)-1,1,3,3-tetramethyluronium hexafluorophosphate) (0.120 g, 0.32 mmol). The resulting mixture was stirred at room temperature for 2 hours. The reaction mixture was poured into water (20 mL) and extracted with with ethyl acetate (15 mL×3). The combined organic layers were washed with water (15 mL) and brine (10 mL), dried over anhydrous sodium sulfate, and concentrated under reduced pressure to give a crude residue which was purified by silica gel column chromatography using a 6% methanol in dichloromethane gradient to afford tert-butyl 1-((((2R,3S)-4-oxo-4-(4-(thiazol-2-yl)piperidin-1-yl)-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylate (0.100 g, 48% yield) as a light yellow solid. MS: [MH]⁺ 992.30.

Synthesis of 1-((((2R,3S)-4-oxo-4-(4-(thiazol-2-yl)piperidin-1-yl)-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylic acid (I-76): To a solution of tert-butyl 1-((((2R,3S)-4-oxo-4-(4-(thiazol-2-yl)piperidin-1-yl)-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylate (0.100 g, 0.10 mmol) in dichloromethane (6.0 mL) added 2,2,2-trifluoroacetic acid (2.0 mL), the resulting mixture was stirred at room temperature for 2 hours. The reaction mixture was concentrated under reduced pressure. The residue was taken up in dichloromethane (15 mL), washed with water (10 mL×2), dried over anhydrous sodium sulfate, and concentrated under reduced pressure to give a crude residue which was purified by silica gel column chromatography using a 9% methanol in dichloromethane gradient to afford 1-((((2R,3S)-4-oxo-4-(4-(thiazol-2-yl)piperidin-1-yl)-3-((S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamido)butan-2-yl)oxy)methyl)cyclohexanecarboxylic acid (0.066 g, 70% yield) as a white solid. ¹HNMR (400 MHz, CD₃OD): δ 8.29-8.13 (m, 2H), 7.96-7.93 (m, 1H), 7.71-7.64 (m, 3H), 7.48-7.40 (m, 2H), 5.49-5.48 (m, 2H), 4.95-4.91 (m, 1H), 4.59-3.75 (m, 11H), 3.63-3.58 (m, 1H), 3.48-3.42 (m, 3H), 2.89-2.83 (m, 1H), 2.23-2.12 (m, 2H), 2.03-1.99 (m, 2H), 1.86-1.66 (m, 2H), 1.54 (br, 3H), 1.45-1.40 (m, 2H), 1.34-1.23 (m, 7H), 1.17-1.13 (m, 3H). MS: [MH]⁺ 936.65.

### Synthesis of (S)—N-((3S,4R)-4-((2-oxabicyclo[2.2.2]octan-4-yl)methoxy)-2-hydroxy-2-methylpentan-3-yl)-2-(2-cyclopropyl-2-methylpropanoyl)-6-(oxazole-5-carbonyl)-2,6-diazaspiro[3.4]octane-8-carboxamide and (8S)-2-(2-cyclopropyl-2-methylpropanoyl)-N-((3S,4R)-2-hydroxy-4-((1-(hydroxymethyl)cyclohex-3-en-1-yl)methoxy)-2-methylpentan-3-yl)-6-(oxazole-5-carbonyl)-2,6-diazaspiro[3.4]octane-8-carboxamide (I-57)

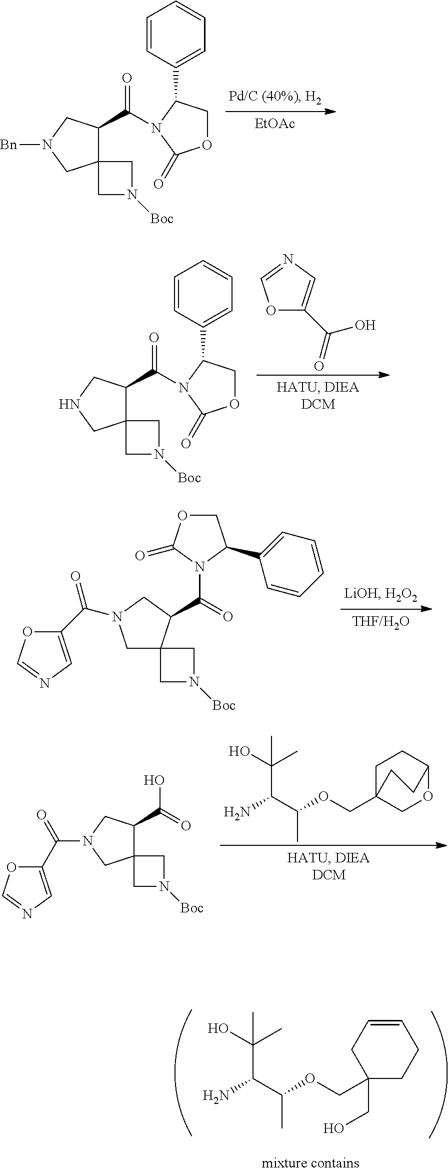

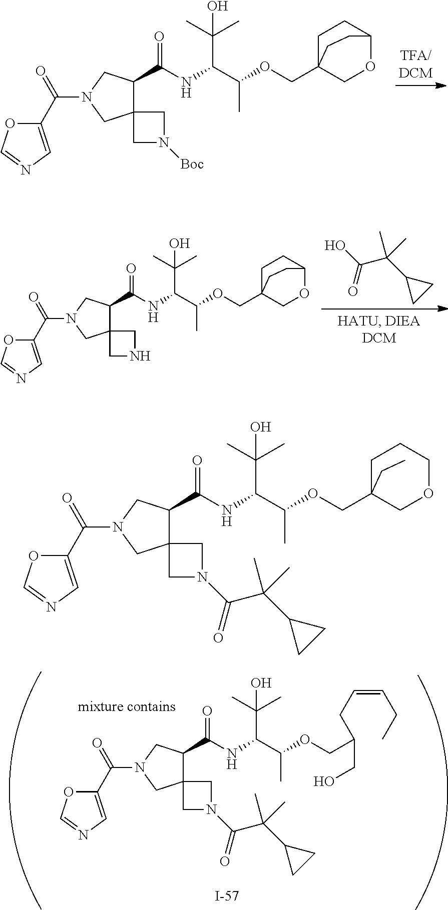

Step 1: tert-butyl (S)-8-((R)-2-oxo-4-phenyloxazolidine-3-carbonyl)-2,6-diazaspiro[3.4]octane-2-carboxylate: To a solution of tert-butyl (S)-6-benzyl-8-((R)-2-oxo-4-phenyloxazolidine-3-carbonyl)-2,6-diazaspiro[3.4]octane-2-carboxylate (1.0 g, 2.03 mmol) in EtOAc (8 mL) was added 10% Pd/C (400 mg) and the reaction stirred under a H₂ atmosphere for 24 h. 50% conversion was observed so the catalyst was removed and the residue resubjected to the same hydrogenation conditions for a further 24 h. The catalyst was then removed by filtration through Celite and the filtrate concentrated to afford tert-butyl (S)-8-((R)-2-oxo-4-phenyloxazolidine-3-carbonyl)-2,6-diazaspiro[3.4]octane-2-carboxylate (800 mg, 98%) which was used directly in the next step. LCMS m/z=402.2 [M+H]⁺; ¹H NMR (400 MHz, DMSO-d₆) δ 7.39-7.25 (m, 5H), 5.45 (dd, J=8.6, 3.6 Hz, 1H), 4.73 (t, J=8.8 Hz, 1H), 4.13 (td, J=8.8, 4.2 Hz, 2H), 3.64 (dd, J=22.8, 8.6 Hz, 4H), 3.04 (d, J=10.8 Hz, 2H), 2.75-2.63 (m, 2H), 1.37 (s, 9H).

Step 2: tert-butyl (S)-6-(oxazole-5-carbonyl)-8-((R)-2-oxo-4-phenyloxazolidine-3-carbonyl)-2,6-diazaspiro[3.4]octane-2-carboxylate: To a solution of oxazole-5-carboxylic acid (191 mg, 1.69 mmol) in DCM (8 mL) was added HATU (644 mg, 1.69 mmol) and DIPEA (657 mg, 5.08 mmol). The reaction mixture was stirred at room temperature for 30 min then tert-butyl (S)-8-((R)-2-oxo-4-phenyloxazolidine-3-carbonyl)-2,6-diazaspiro[3.4]octane-2-carboxylate (680 mg, 1.69 mmol) was added and stirring continued for 2 h. The mixture was diluted with water (10 mL) and extracted with DCM (30 mL×2). The combined organic layers were washed with brine, dried over Na₂SO₄, filtered and concentrated. The residue obtained was purified by column chromatography on silica gel (eluent: DCM:MeOH=100:1) to afford tert-butyl (S)-6-(oxazole-5-carbonyl)-8-((R)-2-oxo-4-phenyloxazolidine-3-carbonyl)-2,6-diazaspiro[3.4]octane-2-carboxylate (400 mg, 47%) as a yellow solid. LCMS m/z=397.1 [M-100+H]⁺; ¹H NMR (400 MHz, DMSO-d₆) δ 8.57 (d, J=32.4 Hz, 1H), 7.77 (d, J=6.6 Hz, 1H), 7.34-7.18 (m, 5H), 5.44 (dd, J=8.6, 4.0 Hz, 1H), 4.81-4.72 (m, 1H), 4.36 (dd, J=40.2, 6.8 Hz, 1H), 4.20-4.11 (m, 2H), 4.03-3.97 (m, 1H), 3.85 (d, J=11.8 Hz, 4H), 3.64-3.58 (m, 1H), 3.18-3.09 (m, 1H), 1.37 (s, 9H).

Step 3: (S)-2-(tert-butoxycarbonyl)-6-(oxazole-5-carbonyl)-2,6-diazaspiro[3.4]octane-8-carboxylic acid: To a solution of tert-butyl (S)-6-(oxazole-5-carbonyl)-8-((R)-2-oxo-4-phenyloxazolidine-3-carbonyl)-2,6-diazaspiro[3.4]octane-2-carboxylate (400 mg, 0.81 mmol) in a mixture of THF and water (8 mL/1 mL) at 0° C. was added a solution of lithium hydroxide monohydrate (70 mg, 1.61 mmol) and 30% H₂O₂ (68 mg, 2.01 mmol) in H₂O (1 mL). The reaction mixture was stirred at 0° C. for 1 h then was diluted with water (20 mL) and extracted with EtOAc (30 mL). The aqueous layer was collected and acidified to pH 2 with 1M HCl then extracted with EtOAc (60 mL×3). The combined organic layers were washed with brine, dried over Na₂SO₄, filtered and concentrated to afford (S)-2-(tert-butoxycarbonyl)-6-(oxazole-5-carbonyl)-2,6-diazaspiro[3.4]octane-8-carboxylic acid (200 mg, 70%) as a white solid which was used directly in the next step. LCMS m/z=252.1 [M+H-100]⁺; ¹H NMR (400 MHz, DMSO-d₆) δ 12.93 (s, 1H), 7.79 (d, J=14.6 Hz, 1H), 4.00-3.52 (m, 8H), 3.24 (m, 1H), 1.37 (s, 9H).

Step 4: tert-butyl (S)-8-(((4R)-4-((2-oxabicyclo[2.2.2]octan-4-yl)methoxy)-2-hydroxy-2-methylpentan-3-yl)carbamoyl)-6-(oxazole-5-carbonyl)-2,6-diazaspiro[3.4]octane-2-carboxylate: To a solution of (S)-2-(tert-butoxycarbonyl)-6-(oxazole-5-carbonyl)-2,6-diazaspiro[3.4]octane-8-carboxylic acid (110 mg, 0.35 mmol) in DCM (2 mL) was added HATU (118 mg, 0.35 mmol) and DIPEA (160 mg, 1.24 mmol). The reaction mixture was stirred at room temperature for 30 min then (4R)-4-((2-oxabicyclo[2.2.2]octan-4-yl)methoxy)-3-amino-2-methylpentan-2-ol (90 mg, 0.35 mmol) was added and stirring continued for 2 h. The mixture was diluted with water (10 mL) and extracted with DCM (30 mL×2). The combined organic layers were washed with brine, dried over Na₂SO₄, filtered and concentrated. The residue was purified by prep-TLC (eluent: DCM:MeOH=20:1) to afford tert-butyl (S)-8-(((4R)-4-((2-oxabicyclo[2.2.2]octan-4-yl)methoxy)-2-hydroxy-2-methylpentan-3-yl)carbamoyl)-6-(oxazole-5-carbonyl)-2,6-diazaspiro[3.4]octane-2-carboxylate (74 mg, 40%) as a colourless oil. LCMS m/z=591.3 [M+H]⁺; ¹H NMR (400 MHz, DMSO-d₆) δ 8.56 (d, J=1.4 Hz, 1H), 7.79 (d, J=15.4 Hz, 1H), 4.35-4.22 (m, 1H), 4.09 (q, J=5.4 Hz, 1H), 3.99-3.77 (m, 5H), 3.65 (dt, J=15.0, 10.8 Hz, 6H), 3.18-3.10 (m, 3H), 1.84 (d, J=12.8 Hz, 1H), 1.79-1.68 (m, 1H), 1.67-1.52 (m, 4H), 1.46 (d, J=12.6 Hz, 2H), 1.36 (s, 9H), 1.10-1.02 (m, 6H), 0.92 (t, J=6.0 Hz, 3H).

Step 5: (S)—N-((4R)-4-((2-oxabicyclo[2.2.2]octan-4-yl)methoxy)-2-hydroxy-2-methylpentan-3-yl)-6-(oxazole-5-carbonyl)-2,6-diazaspiro[3.4]octane-8-carboxamide: To a solution of tert-butyl (S)-8-(((4R)-4-((2-oxabicyclo[2.2.2]octan-4-yl)methoxy)-2-hydroxy-2-methylpentan-3-yl)carbamoyl)-6-(oxazole-5-carbonyl)-2,6-diazaspiro[3.4]octane-2-carboxylate (60 mg, 0.1 mmol) in DCM (2 mL) was added TFA (1 mL) and the reaction stirred at room temperature for 2 h. The solvent was removed under reduced pressure to afford (S)—N-((4R)-4-((2-oxabicyclo[2.2.2]octan-4-yl)methoxy)-2-hydroxy-2-methylpentan-3-yl)-6-(oxazole-5-carbonyl)-2,6-diazaspiro[3.4]octane-8-carboxamide (50 mg, 100%) which was used directly in the next step. LCMS m/z=491.3 [M+H]⁺.

Step 6: (S)—N-((3S,4R)-4-((2-oxabicyclo[2.2.2]octan-4-yl)methoxy)-2-hydroxy-2-methylpentan-3-yl)-2-(2-cyclopropyl-2-methylpropanoyl)-6-(oxazole-5-carbonyl)-2,6-diazaspiro[3.4]octane-8-carboxamide; (8S)-2-(2-cyclopropyl-2-methylpropanoyl)-N-((3S,4R)-2-hydroxy-4-((1-(hydroxymethyl)cyclohex-3-en-1-yl)methoxy)-2-methylpentan-3-yl)-6-(oxazole-5-carbonyl)-2,6-diazaspiro[3.4]octane-8-carboxamide (I-57): To a solution of 2-cyclopropyl-2-methylpropanoic acid (13 mg, 0.1 mmol) in DCM (2 mL) was added HATU (39 mg, 0.1 mmol) and DIPEA (53 mg, 0.4 mmol). The reaction mixture was stirred at room temperature for 30 min then (S)—N-((4R)-4-((2-oxabicyclo[2.2.2]octan-4-yl)methoxy)-2-hydroxy-2-methylpentan-3-yl)-6-(oxazole-5-carbonyl)-2,6-diazaspiro[3.4]octane-8-carboxamide (50 mg, 0.1 mmol) was added and stirring continued for 2 h. The solvent was removed under reduced pressure and the residue obtained purified by prep-HPLC to afford (5.7 mg, 9%) as a white solid as the first eluting compound. LCMS m/z=601.5 [M+H]⁻; ¹H NMR (400 MHz, Methanol-d₄) δ 8.38 (d, J=3.2 Hz, 1H), 7.79 (d, J=14.6 Hz, 1H), 4.55-4.20 (m, 2H), 4.18-3.92 (m, 4H), 3.83 (s, 2H), 3.75 (s, 2H), 3.54-3.41 (m, 2H), 3.25 (s, 2H), 3.15-3.11 (m, 1H), 3.05-2.98 (m, 1H), 2.03 (d, J=21.8 Hz, 2H), 1.68 (t, J=12.2 Hz, 3H), 1.55 (s, 2H), 1.31-1.22 (m, 3H), 1.19-0.95 (m, 14H), 0.41 (d, J=40.4 Hz, 4H). Further elution provided (8S)-2-(2-cyclopropyl-2-methylpropanoyl)-N-((3S,4R)-2-hydroxy-4-((1-(hydroxymethyl)cyclohex-3-en-1-yl)methoxy)-2-methylpentan-3-yl)-6-(oxazole-5-carbonyl)-2,6-diazaspiro[3.4]octane-8-carboxamide (7 mg, 11%) as a white solid as the second eluting compound. LCMS m/z=601.4 [M+H]⁺; ¹H NMR (400 MHz, Methanol-d₄) δ 8.38 (d, J=3.4 Hz, 1H), 7.79 (d, J=13.6 Hz, 1H), 5.73-5.55 (m, 2H), 4.54 (d, J=22.0 Hz, 1H), 4.39-3.98 (m, 5H), 3.85 (d, J=32.2 Hz, 3H), 3.53-3.40 (m, 4H), 3.28-3.16 (m, 1H), 2.05 (d, J=9.6 Hz, 2H), 1.86 (s, 2H), 1.65-1.50 (m, 2H), 1.34-0.91 (m, 18H), 0.41 (dd, J=38.4, 6.8 Hz, 4H).

### Synthesis of (S)—N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazole-5-carbonyl)-2,6-diazaspiro[3.4]octane-8-carboxamide and (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazole-5-carbonyl)-2,6-diazaspiro[3.4]octane-8-carboxamide (I-52)

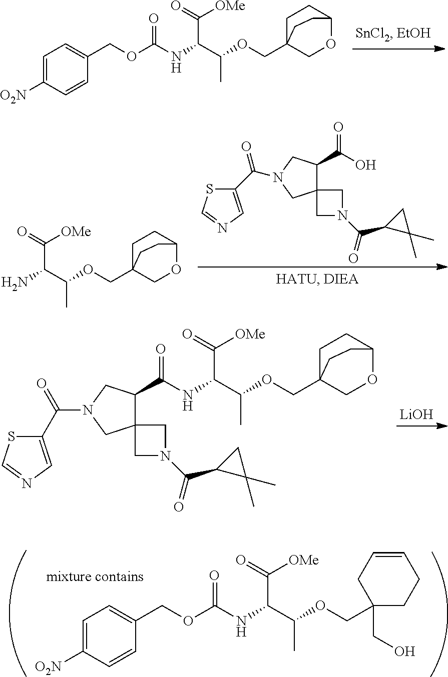

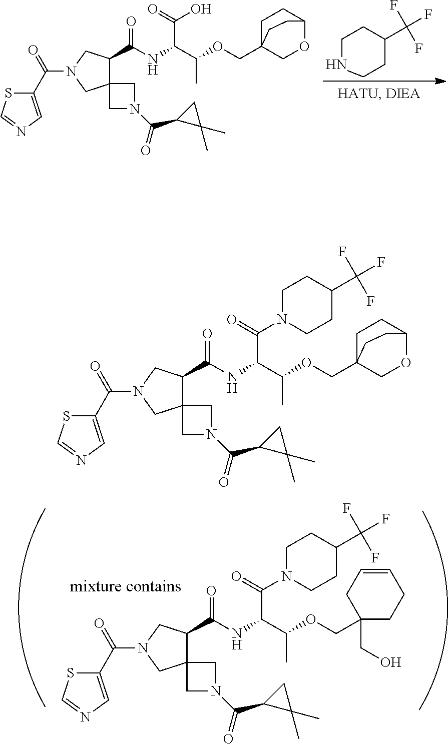

Step 1: methyl O-((2-oxabicyclo[2.2.2]octan-4-yl)methyl)-L-threoninate: To a solution of methyl O-((2-oxabicyclo[2.2.2]octan-4-yl)methyl)-N-(((4-nitrobenzyl)oxy)carbonyl)-L-threoninate (1.75 g, 4.0 mmol) in absolute ethanol (10 mL) was added tin(II) chloride (3.8 g, 20 mmol). The reaction was heated at reflux for 2 h then cooled to room temperature and the solvent removed under reduced pressure. The residue was purified on an Al₂O₃ column (eluent: DCM:MeOH=20:1) to afford methyl O-((2-oxabicyclo[2.2.2]octan-4-yl)methyl)-L-threoninate (940 mg, 94%) as a pale yellow oil. LCMS m/z=258.1 [M+H]⁺.

Step 2: methyl O-((2-oxabicyclo[2.2.2]octan-4-yl)methyl)-N—((S)-2-((S)-2,2-dimethylcyclopropane-1-carbonyl)-6-(thiazole-5-carbonyl)-2,6-diazaspiro[3.4]octane-8-carbonyl)-L-threoninate: To a solution of (S)-2-((S)-2,2-dimethylcyclopropane-1-carbonyl)-6-(thiazole-5-carbonyl)-2,6-diazaspiro[3.4]octane-8-carboxylic acid (835 mg, 2.3 mmol) in DCM (6 mL) was added HATU (874 mg, 2.3 mmol) and DIPEA (742 mg, 5.75 mmol) and the mixture stirred at room temperature for 30 min. Methyl O-((2-oxabicyclo[2.2.2]octan-4-yl)methyl)-L-threoninate (600 mg, 2.3 mmol) was added and the reaction stirred for another 2 h. The reaction was quenched with water (30 mL) and extracted with DCM (40 mL×3). The combined organic layers were washed with brine, dried over Na₂SO₄, filtered and concentrated. The residue obtained was purified by column chromatography on silica gel (eluent: DCM:MeOH=50:1) to afford methyl O-((2-oxabicyclo[2.2.2]octan-4-yl)methyl)-N—((S)-2-((S)-2,2-dimethylcyclopropane-1-carbonyl)-6-(thiazole-5-carbonyl)-2,6-diazaspiro[3.4]octane-8-carbonyl)-L-threoninate (850 mg, 62%) as a yellow oil. LCMS m/z=603.1 [M+H]⁺; ¹H NMR (400 MHz, CDCl₃) δ 8.91 (s, 1H), 8.27 (s, 1H), 4.70-4.57 (m, 1H), 4.40-3.96 (m, 10H), 3.79-3.72 (m, 4H), 3.65-3.58 (m, 1H), 3.52-3.42 (m, 1H), 3.23-3.09 (m, 2H), 2.05-1.94 (m, 2H), 1.58-1.51 (m, 4H), 1.48-1.41 (m, 2H), 1.19-1.11 (m, 12H), 0.83-0.71 (m, 1H).

Step 3: O-((2-oxabicyclo[2.2.2]octan-4-yl)methyl)-N—((S)-2-((S)-2,2-dimethylcyclopropane-1-carbonyl)-6-(thiazole-5-carbonyl)-2,6-diazaspiro[3.4]octane-8-carbonyl)-L-threonine: To a solution of methyl O-((2-oxabicyclo[2.2.2]octan-4-yl)methyl)-N—((S)-2-((S)-2,2-dimethylcyclopropane-1-carbonyl)-6-(thiazole-5-carbonyl)-2,6-diazaspiro[3.4]octane-8-carbonyl)-L-threoninate (850 mg, 1.41 mmol) in a mixture of THF (8 mL), MeOH (2 mL) and water (2 mL) was added LiOH (97 mg, 4.23 mmol). The reaction was stirred for 4 h then was diluted with water (50 mL) and extracted with EtOAc (30 mL×2). The aqueous phase was acidified to pH 2 with 1M HCl and extracted with EtOAc (50 mL×3). The combined organic layers were washed with brine, dried over Na₂SO₄ filtered and concentrated to afford O-((2-oxabicyclo[2.2.2]octan-4-yl)methyl)-N—((S)-2-((S)-2,2-dimethylcyclopropane-1-carbonyl)-6-(thiazole-5-carbonyl)-2,6-diazaspiro[3.4]octane-8-carbonyl)-L-threonine (600 mg, 72%) as a yellow oil. LCMS m/z=589.2 [M+H]⁺.

Step 4: (S)—N-((2S,3R)-3-((2-oxabicyclo[2.2.2]octan-4-yl)methoxy)-1-oxo-1-(4-(trifluoromethyl) piperidin-1-yl)butan-2-yl)-2-((S)-2,2-dimethylcyclopropane-1-carbonyl)-6-(thiazole-5-carbonyl)-2,6-diazaspiro[3.4]octane-8-carboxamide; (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazole-5-carbonyl)-2,6-diazaspiro[3.4]octane-8-carboxamide (I-52): To a solution of O-((2-oxabicyclo[2.2.2]octan-4-yl)methyl)-N—((S)-2-((S)-2,2-dimethylcyclopropane-1-carbonyl)-6-(thiazole-5-carbonyl)-2,6-diazaspiro[3.4]octane-8-carbonyl)-L-threonine (50 mg, 0.085 mmol) in DCM (2 mL) was added HATU (32 mg, 0.085 mmol) and DIPEA (27 mg, 0.213 mmol). The mixture was stirred at room temperature for 30 min then 4-(trifluoromethyl)piperidine (14 mg, 0.094 mmol) was added and the reaction stirred for another 2 h. The solvent was removed under reduced pressure and the residue obtained was purified by prep-HPLC to afford (S)—N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(thiazole-5-carbonyl)-2,6-diazaspiro[3.4]octane-8-carboxamide (20 mg, 33%) as the first eluting compound. LCMS m/z=724.4 [M+H]⁺; ¹H NMR (400 MHz, CD₃OD) δ 9.15 (s, 1H), 8.37 (m, 1H), 4.95 (s, 1H), 4.58 (m, 1H), 4.43-3.82 (m, 10H), 3.72 (m, 3H), 3.45 (m, 1H), 3.27-3.13 (m, 2H), 3.02 (s, 1H), 2.69 (s, 1H), 2.51 (s, 1H), 1.96 (m, 4H), 1.65 (m, 4H), 1.57-1.36 (m, 5H), 1.21-1.10 (m, 9H), 1.05 (s, 1H), 0.79 (m, 1H). further elution provided (8S)-2-((S)-2,2-dimethylcyclopropane-1-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-6-(thiazole-5-carbonyl)-2,6-diazaspiro[3.4]octane-8-carboxamide (I-52) LCMS m/z=724.4 [M+H]⁺; ¹H NMR (400 MHz, CD₃OD) δ 9.16 (s, 1H), 8.37 (d, J=10.6 Hz, 1H), 5.69-5.53 (m, 2H), 4.98-4.89 (m, 1H), 4.68-4.57 (m, 1H), 4.40-3.88 (m, 9H), 3.81-3.72 (m, 1H), 3.55-3.39 (m, 4H), 3.21-3.12 (m, 1H), 2.78-2.62 (m, 1H), 2.58-2.44 (m, 1H), 2.08-1.32 (m, 13H), 1.24-1.10 (m, 9H), 1.08-1.02 (m, 1H), 0.85-0.74 (m, 1H).

Table I-1: The compounds listed in Table I-1 were synthesized according to the procedures outlined for I-52 using the appropriate commercially available reagents and/or intermediates described herein.

TABLE I-1

| Example | | | |
|---|---|---|---|
| Number | Compound | ¹H NMR | LCMS |
| I-53 | 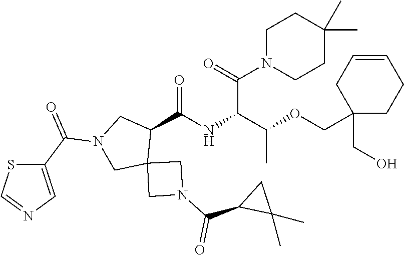 [SMILES: C[C@@H](OCC1(CO)CC=CCC1)[C@H](NC(=O)[C@@H]1CN(C(=O)c2cncs2)CC12CN(C(=O)[C@H]1CC1(C)C)C2)C(=O)N1CCC(C)(C)CC1] | ¹H NMR (400 MHz, Methanol-d₄) δ 9.16 (s, 1H), 8.37 (d, J = 10.8 Hz, 1H), 5.70-5.55 (m, 2H), 4.92 (s, 1H), 4.57 (s, 1H), 4.39 (t, 1H), 4.32-4.23 (m, 1H), 4.19-4.01 (m, 3H), 3.97-3.84 (m, 2H), 3.77 (d, 1H), 3.57 (s, 4H), 3.44 (q, J = 13.1 Hz, 3H), 3.27-3.20 (m, 1H), 2.00 (s, 2H), 1.82 (d, J = 13.9 Hz, 2H), 1.53 (t, J = 6.3 Hz, 2H), 1.47- 1.43 (m, 2H), 1.32 (d, J = 22.1 Hz, 4H), 1.21-1.09 (m, 10H), 1.01 (s, 4H), 0.98 (s, 3H), 0.81-0.76 (m, 1H). | m/z = 684.4 [M + H]⁻ |
| I-54 | 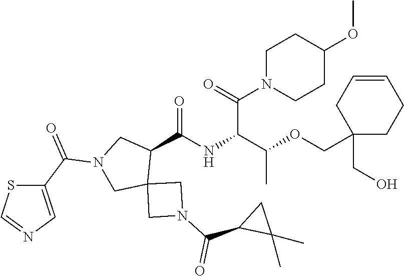 [SMILES: COC1CCN(C(=O)[C@@H](NC(=O)[C@@H]2CN(C(=O)c3cncs3)CC23CN(C(=O)[C@H]2CC2(C)C)C3)[C@@H](C)OCC2(CO)CC=CCC2)CC1] | ¹H NMR (400MHz, Methanol-d4) δ 9.15 (s, 1H), 8.37 (d, J = 11.4 Hz, 1H), 5.70-5.55 (m, 2H), 4.93 (s, 1H), 4.44- 4.32 (m, 1H), 4.31-4.12 (m, 3H), 4.05 (d, J = 19.6 Hz, 2H), 3.99-3.86 (m, 3H), 3.76 (s, 3H), 3.49-3.41 (m, 5H), 3.35 (d, J = 6.3 Hz, 3H), 3.24 (t, J = 9.5 Hz, 1H), 2.04-1.97 (m, 2H), 1.82 (d, J = 16.2 Hz, 3H), 1.61 (s, 1H), 1.52 (s, 2H), 1.44 (d, J = 23.9 Hz, 3H), 1.23-1.09 (m, 11H), 1.08-1.01 (m, 1H), 0.82-0.75 (m, 1H). | m/z = 686.4 [M + H]⁻ |

### Synthesis of (8S)-2-(2-cyclopropyl-2-methylpropanoyl)-6-(2,4-dimethylthiazole-5-carbonyl)-N-((4R)-2-hydroxy-4-((1-(hydroxymethyl)cyclohex-3-en-1-yl)methoxy)-2-methyl pentan-3-yl)-2,6-diazaspiro[3.4]octane-8-carboxamide (1-56)

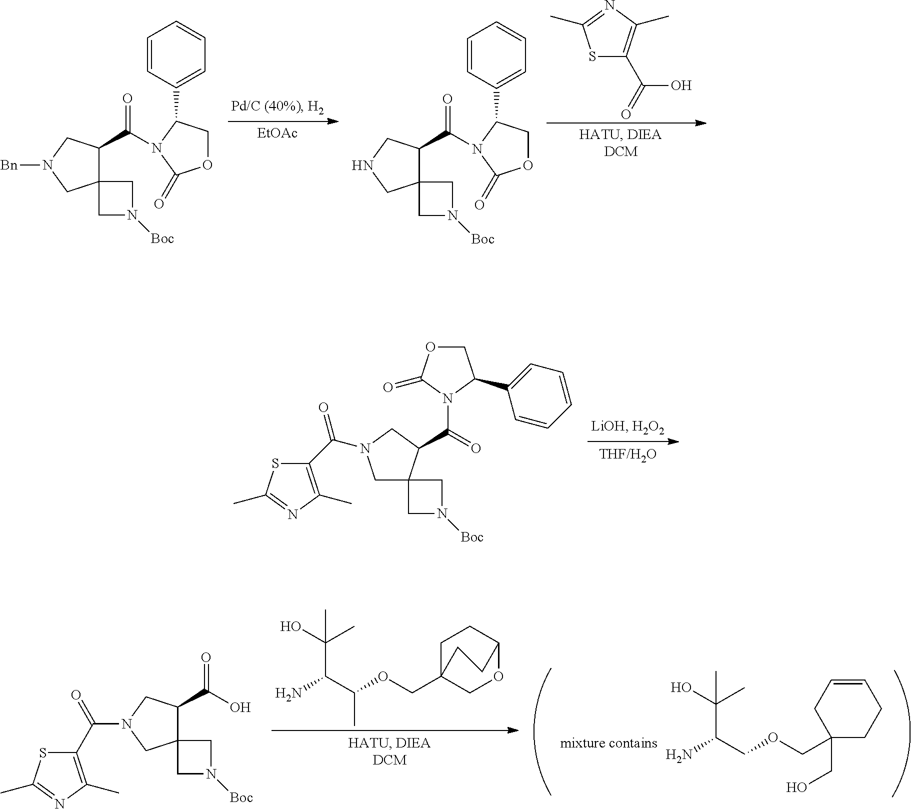

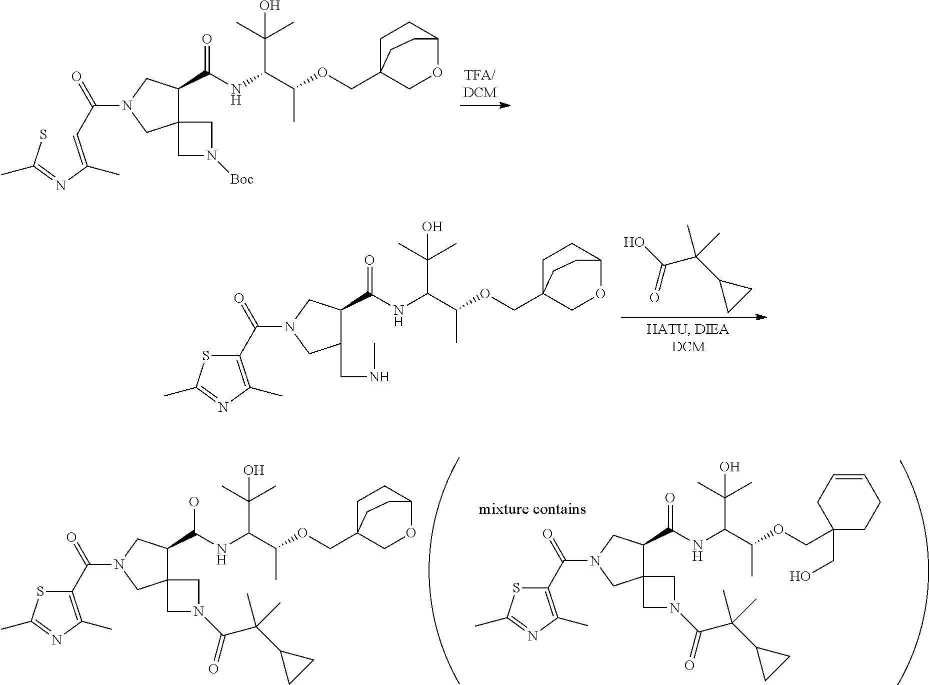

Step 1: tert-butyl (S)-8-((R)-2-oxo-4-phenyloxazolidine-3-carbonyl)-2,6-diazaspiro[3.4]octane-2-carboxylate: To a solution of tert-butyl (S)-6-benzyl-8-((R)-2-oxo-4-phenyloxazolidine-3-carbonyl)-2,6-diazaspiro[3.4]octane-2-carboxylate (1 g, 2 mmol) in EtOAc (8 mL) was added 10% Pd/C (400 mg). The reaction mixture was stirred under a H₂ atmosphere for 24 h, ~50% conversion was observed. The catalyst was removed and the residue resubjected to hydrogenation under the same conditions for a further 24 h. The catalyst was removed by filtration through celite and the filtrate concentrated to afford tert-butyl (S)-8-((R)-2-oxo-4-phenyloxazolidine-3-carbonyl)-2,6-diazaspiro[3.4]octane-2-carboxylate (817 mg) which was used directly in the next step. LCMS m/z=402.2 [M+H]⁺.

Step 2: tert-butyl (S)-6-(2,4-dimethylthiazole-5-carbonyl)-8-((R)-2-oxo-4-phenyloxazolidine-3-carbonyl)-2,6-diazaspiro[3.4]octane-2-carboxylate: To a solution of 2,4-dimethylthiazole-5-carboxylic acid (236 mg, 1.5 mmol) in DCM (10 mL) was added HATU (570 mg, 1.5 mmol) and the reaction stirred at room temperature for 30 min. tert-butyl (S)-8-((R)-2-oxo-4-phenyloxazolidine-3-carbonyl)-2,6-diazaspiro[3.4]octane-2-carboxylate (600 mg, 1.5 mmol) and DIPEA (581 mg, 3 mmol) were added and the reaction stirred for another 2 h then diluted with water (20 mL) and extracted with DCM (50 mL×3). The combined organic layers were washed with brine, dried over Na₂SO₄, filtered and concentrated. The residue obtained was purified by column chromatography on silica gel (eluent: DCM:MeOH=50:1) to afford tert-butyl (S)-6-(2,4-dimethylthiazole-5-carbonyl)-8-((R)-2-oxo-4-phenyloxazolidine-3-carbonyl)-2,6-diazaspiro[3.4]octane-2-carboxylate (450 mg, 56%) as a yellow solid. LCMS m/z=541.2 [M+H]⁺; 1H NMR (400 MHz, Chloroform-d) δ 7.43-7.30 (m, 4H), 5.41 (dd, J=8.9, 4.0 Hz, 1H), 4.79-4.72 (m, 1H), 4.40-4.28 (m, 2H), 3.98-3.85 (m, 5H), 3.79-3.68 (m, 3H), 2.80 (s, 6H), 1.43 (s, 9H).

Step 3: (S)-2-(tert-butoxycarbonyl)-6-(2,4-dimethylthiazole-5-carbonyl)-2,6-diazaspiro[3.4]octane-8-carboxylic acid: To a solution of tert-butyl (S)-6-(2,4-dimethylthiazole-5-carbonyl)-8-((R)-2-oxo-4-phenyloxazolidine-3-carbonyl)-2,6-diazaspiro[3.4]octane-2-carboxylate (350 mg, 0.65 mmol) in a mixture of THF and water (4 mL/1 mL) at 0° C. was added a solution of lithium hydroxide monohydrate (55 mg, 1.3 mmol) and 30% H₂O₂ (184 mg, 1.6 mmol) in water (1 mL). The reaction mixture was stirred at 0° C. for 1 h then diluted with water (25 mL) and extracted with EtOAc (30 mL). The aqueous layer was collected and acidified to pH 2 with 1M HCl then extracted with EtOAc (80 mL×3). The combined organic layers were washed with brine, dried over Na₂SO₄, filtered and concentrated to afford (S)-2-(tert-butoxycarbonyl)-6-(2,4-dimethylthiazole-5-carbonyl)-2,6-diazaspiro[3.4]octane-8-carboxylic acid (250 mg, 76%) as a white solid which was used directly in the next step. LCMS m/z=396.1 [M+H]⁺; 1H NMR (400 MHz, DMSO-d6) δ 4.13-4.05 (m, 1H), 3.95-3.84 (m, 2H), 3.79-3.67 (m, 4H), 3.64-3.57 (m, 2H), 2.69 (s, 3H), 2.62 (s, 3H), 1.37 (s, 9H).

Step 4: tert-butyl (S)-8-(((3S,4R)-4-((2-oxabicyclo[2.2.2]octan-4-yl)methoxy)-2-hydroxy-2-methylpentan-3-yl)carbamoyl)-6-(2,4-dimethylthiazole-5-carbonyl)-2,6-diazaspiro[3.4]octane-2-carboxylate: To a solution of (S)-2-(tert-butoxycarbonyl)-6-(2,4-dimethylthiazole-5-carbonyl)-2,6-diazaspiro[3.4]octane-8-carboxylic acid (130 mg, 0.4 mmol) in DCM (5 mL) was added HATU (152 mg, 0.4 mmol) and DIPEA (155 mg, 1.2 mmol). The mixture was stirred at room temperature for 30 min then (3S,4R)-4-((2-oxabicyclo[2.2.2]octan-4-yl)methoxy)-3-amino-2-methylpentan-2-ol (100 mg, 0.5 mmol) was added and stirring continued for 2 h. The reaction was quenched with water (10 mL) and extracted with DCM (50 mL×3). The combined organic layers were washed with brine, dried over Na₂SO₄, filtered and concentrated. The residue obtained was purified by prep-TLC (DCM:MeOH=20:1) to afford tert-butyl (S)-8-(((3S,4R)-4-((2-oxabicyclo[2.2.2]octan-4-yl)methoxy)-2-hydroxy-2-methylpentan-3-yl)carbamoyl)-6-(2,4-dimethylthiazole-5-carbonyl)-2,6-diazaspiro[3.4]octane-2-carboxylate (90 mg, 43%) as a yellow solid. LCMS m/z=635.3 [M+H]⁺; ¹H NMR (400 MHz, DMSO-d6) δ 7.48 (s, 1H), 5.60 (s, 1H), 4.37-4.11 (m, 2H), 4.06-3.36 (m, 14H), 2.62 (s, 3H), 2.34 (s, 3H), 1.49-1.43 (m, 2H), 1.41-1.34 (m, 10H), 1.31-1.23 (m, 5H), 1.14-1.02 (m, 6H), 0.99-0.89 (m, 3H).

Step 5: (S)—N-((3S,4R)-4-((2-oxabicyclo[2.2.2]octan-4-yl)methoxy)-2-hydroxy-2-methylpentan-3-yl)-6-(2,4-dimethylthiazole-5-carbonyl)-2,6-diazaspiro[3.4]octane-8-carboxamide: To a solution of tert-butyl (S)-8-(((3S,4R)-4-((2-oxabicyclo[2.2.2]octan-4-yl)methoxy)-2-hydroxy-2-methylpentan-3-yl)carbamoyl)-6-(2,4-dimethylthiazole-5-carbonyl)-2,6-diazaspiro[3.4]octane-2-carboxylate (60 mg, 0.1 mmol) in DCM (4 mL) was added TFA (1 mL). The reaction was stirred at room temperature for 1 h then the solvent was removed under reduced pressure to afford (S)—N-((3S,4R)-4-((2-oxabicyclo[2.2.2]octan-4-yl)methoxy)-2-hydroxy-2-methylpentan-3-yl)-6-(2,4-dimethylthiazole-5-carbonyl)-2,6-diazaspiro[3.4]octane-8-carboxamide (50 mg, 100%) which was used directly in the next step. LCMS m/z=535.3 [M+H]⁺.

Step 6: (S)—N-((4R)-4-((2-oxabicyclo[2.2.2]octan-4-yl)methoxy)-2-hydroxy-2-methylpentan-3-yl)-2-(2-cyclopropyl-2-methylpropanoyl)-6-(2,4-dimethylthiazole-5-carbonyl)-2,6-diazaspiro[3.4]octane-8-carboxamide(8S)-2-(2-cyclopropyl-2-methylpropanoyl)-6-(2,4-dimethylthiazole-5-carbonyl)-N-((4R)-2-hydroxy-4-((1-(hydroxymethyl)cyclohex-3-en-1-yl)methoxy)-2-methylpentan-3-yl)-2,6-diazaspiro[3.4]octane-8-carboxamide (1-56): To a solution of 2-cyclopropyl-2-methylpropanoic acid (12 mg, 0.1 mmol) in DCM (4 mL) was added HATU (38 mg, 0.1 mmol) and DIPEA (52 mg, 0.4 mmol). The mixture was stirred at room temperature for 30 min then (S)—N-((4R)-4-((2-oxabicyclo[2.2.2]octan-4-yl)methoxy)-2-hydroxy-2-methylpentan-3-yl)-6-(2,4-dimethylthiazole-5-carbonyl)-2,6-diazaspiro[3.4]octane-8-carboxamide (50 mg, 0.1 mmol) was added and the reaction stirred for another 2 h. The reaction was quenched with water (10 mL) and extracted with DCM (50 mL) and the combined organic layers were washed with brine, dried over Na₂SO₄, filtered and concentrated. The residue obtained was purified by prep-HPLC to afford (8S)-2-(2-cyclopropyl-2-methylpropanoyl)-6-(2,4-dimethylthiazole-5-carbonyl)-N-((4R)-2-hydroxy-4-((1-(hydroxymethyl)cyclohex-3-en-1-yl)methoxy)-2-methylpentan-3-yl)-2,6-diazaspiro[3.4]octane-8-carboxamide (1-56) (30 mg, 50%) as a white solid. LCMS m/z=645.5 [M+H]⁺; ¹H NMR (400 MHz, DMSO-d6) δ 7.68-7.48 (m, 1H), 5.65-5.50 (m, 2H), 4.56-4.17 (m, 4H), 3.99-3.40 (m, 9H), 3.29-3.01 (m, 4H), 2.62 (s, 3H), 2.49 (s, 2H), 2.34 (s, 3H), 1.86 (d, J=56.6 Hz, 4H), 1.49-1.38 (m, 2H), 1.19-0.81 (m, 16H), 0.30 (d, J=34.9 Hz, 4H).

### Synthesis of (S)—N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(2,4-dimethylthiazole-5-carbonyl)-2,6-diazaspiro[3.4]octane-8-carboxamide and (8S)-2-((S)-2,2-dimethylcyclopropane-1-carbonyl)-6-(2,4-dimethylthiazole-5-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-2,6-diazaspiro[3.4]octane-8-carboxamide (I-50)

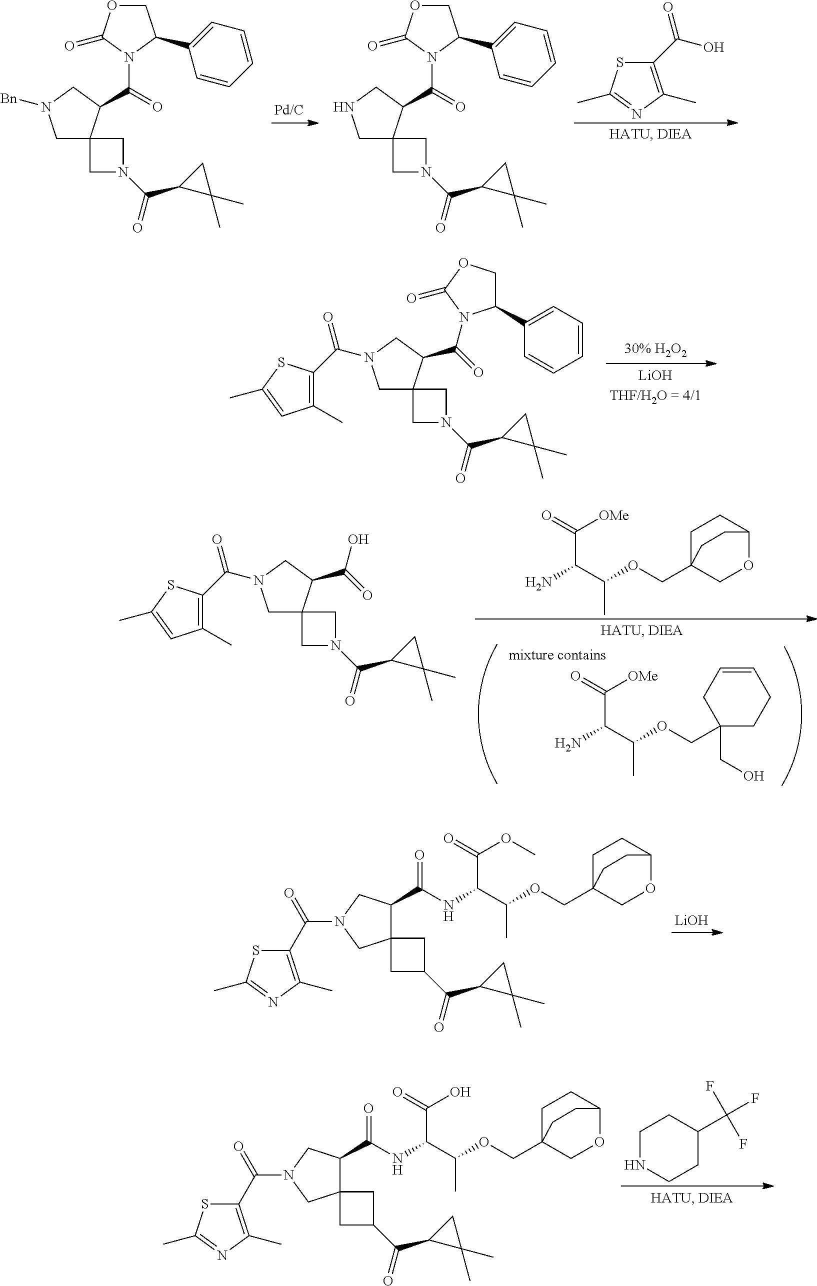

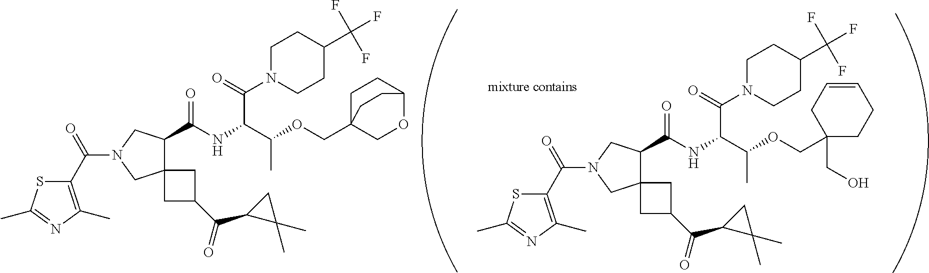

Step 1: (R)-3-((S)-2-((S)-2,2-dimethylcyclopropane-1-carbonyl)-2,6-diazaspiro[3.4]octane-8-carbonyl)-4-phenyloxazolidin-2-one: To a solution of (R)-3-((S)-6-benzyl-2-((S)-2,2-dimethylcyclopropane-1-carbonyl)-2,6-diazaspiro[3.4]octane-8-carbonyl)-4-phenyloxazolidin-2-one (1.0 g, 2.1 mmol) in EtOAc (8 mL) was added 10% Pd/C (400 mg). The reaction mixture was stirred under a H₂ atmosphere for 24 h, ~50% conversion was observed. The catalyst was removed and the residue obtained resubjected to the same hydrogenation conditions for a further 24 h. The catalyst was removed by filtration through Celite and the filtrate concentrated to afford (R)-3-((S)-2-((S)-2,2-dimethylcyclopropane-1-carbonyl)-2,6-diazaspiro[3.4]octane-8-carbonyl)-4-phenyloxazolidin-2-one (750 mg, 920%) which was used directly in the next step. LCMS m/z=398.1 [M+H]⁺.

Step 2: (R)-3-((S)-2-((S)-2,2-dimethylcyclopropane-1-carbonyl)-6-(2,4-dimethylthiazole-5-carbonyl)-2,6-diazaspiro[3.4]octane-8-carbonyl)-4-phenyloxazolidin-2-one: To a solution of 2,4-dimethylthiazole-5-carboxylic acid (597 mg, 3.8 mmol) in DCM (20 mL) was added HATU (1.4 g, 3.8 mmol) and DIPEA (1.5 g, 11.4 mmol) and the mixture stirred at room temperature for 30 min. (R)-3-((S)-2-((S)-2,2-dimethylcyclopropane-1-carbonyl)-2,6-diazaspiro[3.4]octane-8-carbonyl)-4-phenyloxazolidin-2-one (1.5 g, 3.8 mmol) was added and stirring continued for 3 h. The mixture was diluted with water (30 mL) and extracted with DCM (100 mL×2). The combined organic layers were washed with brine, dried over Na₂SO₄, filtered and concentrated. The mixture was purified by column chromatography on silica gel (eluent: DCM:MeOH=30:1) to afford (R)-3-((S)-2-((S)-2,2-dimethylcyclopropane-1-carbonyl)-6-(2,4-dimethylthiazole-5-carbonyl)-2,6-diazaspiro[3.4]octane-8-carbonyl)-4-phenyloxazolidin-2-one (1.1 g, 55%) as a yellow solid. LCMS m/z=537.2 [M+H]⁺; ¹H NMR (400 MHz, DMSO-d6) δ 7.37-7.20 (m, 5H), 5.50-5.41 (m, 1H), 4.82-4.69 (m, 1H), 4.31-4.11 (m, 3H), 3.90-3.54 (m, 7H), 2.63 (s, 3H), 2.15 (s, 3H), 1.15-1.00 (m, 7H), 0.91-0.83 (m, 1H), 0.72-0.64 (m, 1H).

Step 3: (S)-2-((S)-2,2-dimethylcyclopropane-1-carbonyl)-6-(2,4-dimethylthiazole-5-carbonyl)-2,6-diazaspiro[3.4]octane-8-carboxylic acid: To a solution of (R)-3-((S)-2-((S)-2,2-dimethylcyclopropane-1-carbonyl)-6-(2,4-dimethylthiazole-5-carbonyl)-2,6-diazaspiro[3.4]octane-8-carbonyl)-4-phenyloxazolidin-2-one (1 g, 1.9 mmol) in a mixture of THF and H₂O (20 mL/5 mL) at 0° C. was added a solution of lithium hydroxide monohydrate (160 mg, 3.8 mmol) and 30% H₂O₂ (540 mg, 4.8 mmol) in water (1 mL). The reaction mixture was stirred at 0° C. for 1 h then was diluted with water (20 mL) and extracted with EtOAc (30 mL). The aqueous layer was collected, acidified with 1M HCl to pH ~2 and extracted with EtOAc (50 mL×3). The combined organic layers were washed with brine, dried over Na₂SO₄, filtered and concentrated to afford (S)-2-((S)-2,2-dimethylcyclopropane-1-carbonyl)-6-(2,4-dimethylthiazole-5-carbonyl)-2,6-diazaspiro[3.4]octane-8-carboxylic acid (589 mg, 81%) as a yellow solid which was used directly in the next step. LCMS m/z=392.1 [M+H]⁺.

Step 4: methyl O-((2-oxabicyclo[2.2.2]octan-4-yl)methyl)-N—((S)-2-((S)-2,2-dimethylcyclopropane-1-carbonyl)-6-(2,4-dimethylthiazole-5-carbonyl)-2,6-diazaspiro[3.4]octane-8-carbonyl)-L-threoninate: To a solution of (S)-2-((S)-2,2-dimethylcyclopropane-1-carbonyl)-6-(2,4-dimethylthiazole-5-carbonyl)-2,6-diazaspiro[3.4]octane-8-carboxylic acid (250 mg, 0.97 mmol) in DCM (10 mL) was added HATU (369 mg, 0.97 mmol) and DIPEA (375 mg, 2 mmol) and the mixture stirred at room temperature for 30 min. Methyl O-((2-oxabicyclo[2.2.2]octan-4-yl)methyl)-L-threoninate (250 mg, 0.97 mmol) was added and stirring continued for 2 h. The mixture was diluted with water (10 mL) and extracted with DCM (30 mL×2). The combined organic layers were washed with brine, dried over Na₂SO₄, filtered and concentrated. The mixture was purified by prep-HPLC to afford methyl O-((2-oxabicyclo[2.2.2]octan-4-yl)methyl)-N—((S)-2-((S)-2,2-dimethylcyclopropane-1-carbonyl)-6-(2,4-dimethylthiazole-5-carbonyl)-2,6-diazaspiro[3.4]octane-8-carbonyl)-L-threoninate (200 mg, 33%) as a white solid. LCMS m/z=631.3 [M+H]⁺.

Step 5: O-((2-oxabicyclo[2.2.2]octan-4-yl)methyl)-N—((S)-2-((S)-2,2-dimethylcyclopropane-1-carbonyl)-6-(2,4-dimethylthiazole-5-carbonyl)-2,6-diazaspiro[3.4]octane-8-carbonyl)-L-threonine: To a solution of methyl O-((2-oxabicyclo[2.2.2]octan-4-yl)methyl)-N—((S)-2-((S)-2,2-dimethylcyclopropane-1-carbonyl)-6-(2,4-dimethylthiazole-5-carbonyl)-2,6-diazaspiro[3.4]octane-8-carbonyl)-L-threoninate (200 mg, 0.3 mmol) in a mixture of THF, MeOH and H₂O (4 mL/1 mL/1 mL) at 0° C. was added lithium hydroxide monohydrate (27 mg, 3.8 mmol). The reaction was stirred at 0° C. for 1 h then was diluted with water (20 mL) and extracted with EtOAc (30 mL). The aqueous layer was collected, acidified to pH ~2 with 1M HCl and extracted with EtOAc (50 mL×3). The combined organic layers were washed with brine, dried over Na₂SO₄, filtered and concentrated to afford O-((2-oxabicyclo[2.2.2]octan-4-yl)methyl)-N—((S)-2-((S)-2,2-dimethylcyclopropane-1-carbonyl)-6-(2,4-dimethylthiazole-5-carbonyl)-2,6-diazaspiro[3.4]octane-8-carbonyl)-L-threonine (140 mg, 71%) as a yellow solid which was used directly in the next step. LCMS m/z=617.2 [M+H]⁺.

Step 6: (S)—N-((2S,3R)-3-((2-oxabicyclo[2.2.2]octan-4-yl)methoxy)-1-oxo-1-(4-(trifluoromethyl) piperidin-1-yl)butan-2-yl)-2-((S)-2,2-dimethylcyclopropane-1-carbonyl)-6-(2,4-dimethylthiazole-5-carbonyl)-2,6-diazaspiro[3.4]octane-8-carboxamide and (8S)-2-((S)-2,2-dimethylcyclopropane-1-carbonyl)-6-(2,4-dimethylthiazole-5-carbonyl)-N-((2S,3R)-3-((1-(hydroxymethyl)cyclohex-3-en-1-yl)methoxy)-1-oxo-1-(4-(trifluoromethyl)piperidin-1-yl)butan-2-yl)-2,6-diazaspiro[3.4]octane-8-carboxamide (1-50): To a solution of O-((2-oxabicyclo[2.2.2]octan-4-yl)methyl)-N—((S)-2-((S)-2,2-dimethylcyclopropane-1-carbonyl)-6-(2,4-dimethylthiazole-5-carbonyl)-2,6-diazaspiro[3.4]octane-8-carbonyl)-L-threonine (40 mg, 0.06 mmol) in DCM (10 mL) was added HATU (23 mg, 0.18 mmol) and DIPEA (23 mg, 0.18 mmol) and the mixture stirred at room temperature for 30 min. 4-(trifluoromethyl)piperidine (9 mg, 0.06 mmol) was added and stirring continued for 2 h. The reaction was diluted with water (10 mL) and extracted with DCM (30 mL×2). The combined organic layers were washed with brine, dried over Na₂SO₄, filtered and concentrated. The mixture was purified by prep-HPLC to afford (S)—N-((2S,3R)-3-((2-oxabicyclo[2.2.2]octan-4-yl)methoxy)-1-oxo-1-(4-(trifluoromethyl)piperidin-1-yl)butan-2-yl)-2-((S)-2,2-dimethylcyclopropane-1-carbonyl)-6-(2,4-dimethylthiazole-5-carbonyl)-2,6-diazaspiro[3.4]octane-8-carboxamide (14 mg, 29%) as a white solid as the first eluting compound. LCMS m/z=752.4 [M+H]⁺; 1H NMR (400 MHz, DMSO-d6) δ 8.45-8.18 (m, 1H), 4.93-4.77 (m, 1H), 4.55-4.39 (m, 1H), 4.27-3.42 (m, 14H), 3.30-3.05 (m, 2H), 3.02-2.92 (m, 1H), 2.74-2.53 (m, 5H), 2.33 (s, 3H), 1.97-1.70 (m, 4H), 1.60-1.18 (m, 9H), 1.14-0.95 (m, 9H), 0.89-0.80 (m, 1H), 0.71-0.63 (m, 1H); further elution provided (8S)-2-((S)-2,2-dimethylcyclopropane-1-carbonyl)-6-(2,4-dimethylthiazole-5-carbonyl)-N-((2 S,3R)-3-((1-(hydroxymethyl)cyclohex-3-en-1-yl)methoxy)-1-oxo-1-(4-(trifluoromethyl)piperidin-1-yl)butan-2-yl)-2,6-diazaspiro[3.4]octane-8-carboxamide (11 mg, 230%) as a white solid. LCMS m/z=752.4 [M+H]⁺; ¹H NNR (400 MHz, DMSO-d6) δ 8.35-8.22 (m, 1H), 5.65-5.48 (m, 2H), 4.95-4.80 (m, 1H), 4.55-4.42 (m, 1H), 4.31-3.96 (m, 4H), 3.89-3.38 (m, 8H), 3.24-3.09 (m, 3H), 2.70-2.57 (m, 5H), 2.34 (s, 3H), 1.97-1.70 (m, 6H), 1.66-1.14 (m, 6H), 1.14-0.91 (m, 10H), 0.89-0.81 (m, 10H), 0.72-0.62 (m, 1H); (I-50)

Table I-2: The compounds listed in Table I-2 were synthesized according to the procedures outlined for I-50 using the appropriate commercially available reagents and/or intermediates described herein.

TABLE I-2

| Example | | | |
|---|---|---|---|
| Number | Compound | ¹H NMR | LCMS |
| I-55 | 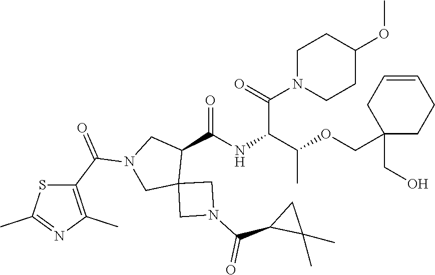 [SMILES: COC1CCN(C(=O)[C@@H](NC(=O)[C@@H]2CN(C(=O)c3sc(C)nc3C)CC23CN(C(=O)[C@H]2CC2(C)C)C3)[C@@H](C)OCC2(CO)CC=CCC2)CC1] | ¹H NMR (400 MHz, DMSO-d₆) δ 5.65-5.48 (m, 2H), 4.89-4.77 (m, 1H), 4.28-3.90 (m, 6H), 3.59 (s, 4H), 3.42-3.38 (m, 1H), 3.33-3.08 (m, 11H), 2.62 (s, 3H), 2.34 (s, 3H), 1.94- 1.72 (m, 6H), 1.58-1.20 (m, 7H), 1.12-1.01 (m, 9H), 0.85 (s, 1H), 0.65 (s, 1H). | m/z = 714.5 [M + H]⁻ |
| I-51 | 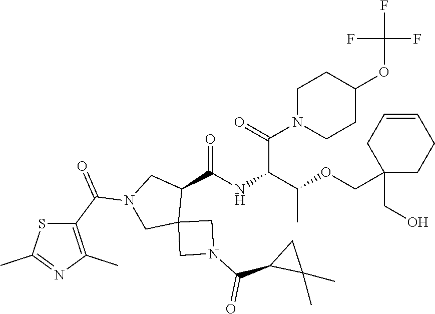 [SMILES: Cc1nc(C)c(C(=O)N2C[C@@H](C(=O)N[C@H](C(=O)N3CCC(OC(F)(F)F)CC3)[C@@H](C)OCC3(CO)CC=CCC3)C3(C2)CN(C(=O)[C@H]2CC2(C)C)C3)s1] | H NMR (400 MHz, DMSO-d₆) δ 8.36-8.22 (m, 1H), 5.63-5.50 (m, 2H), 4.91-4.81 (m, 1H), 4.73-4.64 (m, 1H), 4.30-4.24 (m, 1H), 4.20-4.03 (m, 2H), 3.95-3.78 (m, 4H), 3.75- 3.55 (m, 6H), 3.44-3.38 (m, 2H), 3.22-3.17 (m, 2H), 2.63-2.60 (m, 3H), 2.36-2.32 (m, 3H), 1.99-1.86 (m, 4H), 1.77-1.71 (m, 2H), 1.71- 1.47 (m, 3H), 1.43-1.27 (m, 4H), 1.10-1.00 (m, 9H), 0.87-0.81 (m, 1H), 0.70-0.62 (m, 1H). | m/z = 768.3 [M + H]⁻ |

### Synthesis of N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(spiro[3.5]nonan-7-ylmethyl)-L-threonine (I-40)

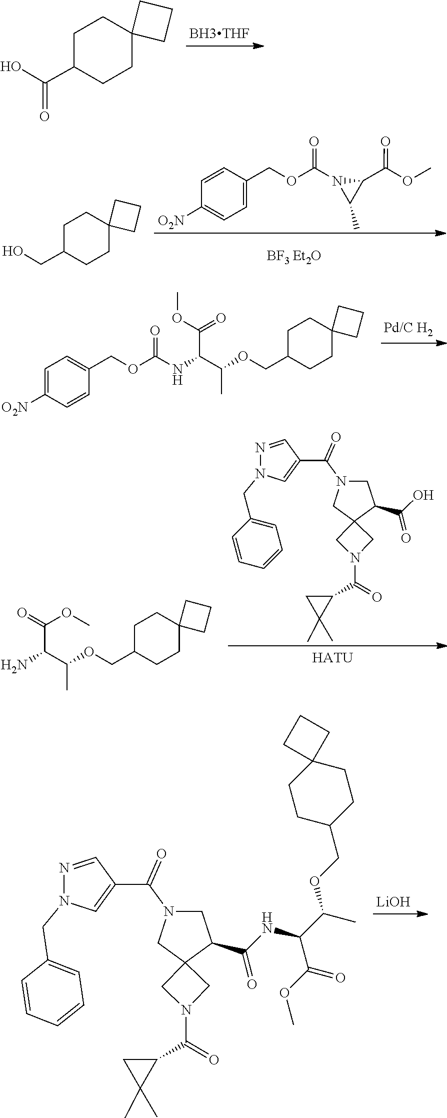

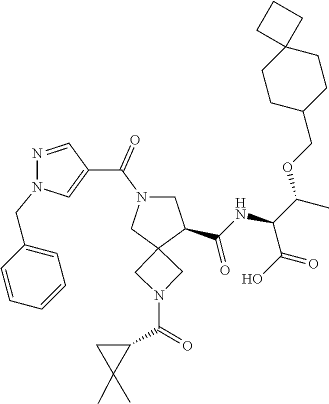 [SMILES: C[C@@H](OCC1CCC2(CCC2)CC1)[C@H](NC(=O)[C@@H]1CN(C(=O)c2cnn(Cc3ccccc3)c2)CC12CN(C(=O)[C@H]1CC1(C)C)C2)C(=O)O]

Step 1: spiro[3.5]nonan-7-ylmethanol: To a solution of spiro[3.5]nonane-7-carboxylic acid (1.50 g, 8.92 mmol) in THE (15 mL) was added BH₃/THF (23 mL, 1.0 M in THF, 22.29 mmol) and the reaction stirred at room temperature overnight. The reaction was quenched with MeOH (5 mL), diluted with water (50 mL) and extracted with EtOAc (50 mL×3). The combined organic layers were washed with brine, dried over Na₂SO₄, filtered and concentrated to afford spiro[3.5]nonan-7-ylmethanol (1.50 g, 100%) as a colorless oil. ¹H NMR (400 MHz, Chloroform-d) δ 3.42 (d, J=6.4 Hz, 2H), 1.85-1.65 (m, 9H), 1.59-1.56 (m, 1H), 1.41-1.36 (m, 1H), 1.29-1.21 (m, 2H), 0.98-0.90 (m, 2H).

Step 2: methyl N-(((4-nitrobenzyl)oxy)carbonyl)-O-(spiro[3.5]nonan-7-ylmethyl)-L-threoninate: To a solution of 2-methyl 1-(4-nitrobenzyl) (2S,3S)-3-methylaziridine-1,2-dicarboxylate (500 mg, 1.70 mmol) and spiro[3.5]nonan-7-ylmethanol (524 mg, 3.40 mmol) in toluene (0.5 mL) at room temperature was added boron trifluoride etherate (482 mg, 3.40 mmol). The reaction was stirred at room temperature for 2 h. then was concentrated and the residue obtained purified by column chromatography on silica gel (eluent: Pet. Ether:EtOAc=4:1) to afford methyl N-(((4-nitrobenzyl)oxy)carbonyl)-O-(spiro[3.5]nonan-7-ylmethyl)-L-threoninate (320 mg, 41%) as a colourless oil. LCMS m/z=449.2 [M+H]⁺; ¹H NMR (400 MHz, DMSO-d6) δ 8.26-8.21 (m, 2H), 7.69-7.57 (m, 3H), 5.20 (s, 2H), 4.25-4.16 (m, 1H), 3.88-3.79 (m, 1H), 3.64 (s, 3H), 3.28-3.21 (m, 1H), 3.06-2.99 (m, 1H), 1.83-1.74 (m, 2H), 1.70-1.58 (m, 6H), 1.53-1.44 (m, 2H), 1.37-1.23 (m, 2H), 1.19-1.15 (m, 1H), 1.11-1.05 (m, 3H), 0.89-0.81 (m, 2H).

Step 3: methyl O-(spiro[3.5]nonan-7-ylmethyl)-L-threoninate: To a solution of methyl N-(((4-nitrobenzyl)oxy)carbonyl)-O-(spiro[3.5]nonan-7-ylmethyl)-L-threoninate (320 mg, 0.71 mmol) in EtOAc (8 mL) was added 10% Pd/C (400 mg). The reaction mixture was stirred under a H₂ atmosphere for 48 h then then the catalyst was removed by filtration through Celite and the filtrate concentrated to afford methyl O-(spiro[3.5]nonan-7-ylmethyl)-L-threoninate (190 mg, 98%) which was used directly in the next step. LCMS m/z=270.3 [M+H]⁺; ¹H NMR (400 MHz, DMSO-d6) δ 6.83-6.78 (m, 1H), 6.48-6.43 (m, 1H), 4.75 (s, 1H), 3.71-3.59 (m, 3H), 3.30-3.14 (m, 3H), 2.12 (s, 1H), 1.83-1.75 (m, 2H), 1.70-1.61 (m, 5H), 1.56-1.42 (m, 2H), 1.40-1.13 (m, 4H), 1.13-1.08 (m, 2H), 0.93-0.84 (m, 2H).

Step 4: methyl N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(spiro[3.5]nonan-7-ylmethyl)-L-threoninate: To a solution of (S)-6-(1-benzyl-1H-pyrazole-4-carbonyl)-2-((S)-2,2-dimethylcyclopropane-1-carbonyl)-2,6-diazaspiro[3.4]octane-8-carboxylic acid (370 mg, 0.85 mmol) in DCM (4 mL) was added HATU (322 mg, 0.85 mmol) and DIPEA (365 mg, 2.82 mmol) and the mixture stirred at room temperature for 30 min. methyl 0-(spiro[3.5]nonan-7-ylmethyl)-L-threoninate (190 mg, 0.71 mmol) was then added and stirring continued for 2 h. The reaction was concentrated and the residue obtained purified by RP-column to afford methyl N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(spiro[3.5]nonan-7-ylmethyl)-L-threoninate (190 mg, 39%) as a yellow oil. LCMS m/z=688.4 [M+H]⁺; ¹H NMR (400 MHz, DMSO-d6) δ 8.60-8.45 (m, 1H), 8.40-8.34 (m, 1H), 7.86-7.79 (m, 1H), 7.37-7.23 (m, 5H), 5.35 (s, 2H), 4.55-4.47 (m, 1H), 4.11-4.05 (m, 1H), 3.99-3.86 (m, 3H), 3.75-3.59 (m, 5H), 3.36-3.25 (m, 4H), 3.06-2.98 (m, 1H), 1.84-1.76 (m, 2H), 1.72-1.61 (m, 6H), 1.55-1.47 (m, 2H), 1.40-1.28 (m, 2H), 1.24-0.96 (m, 12H), 0.91-0.80 (m, 3H), 0.70-0.63 (m, 1H).

Step 5: N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(spiro[3.5]nonan-7-ylmethyl)-L-threonine I-40: To a solution of methyl N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(spiro[3.5]nonan-7-ylmethyl)-L-threoninate (140 mg, 0.20 mmol) in a mixture of THF, water and EtOH (4 mL/1 mL/1 mL) was added LiOH (26 mg, 0.61 mmol). The reaction mixture was stirred at room temperature for 2 h then was diluted with water (20 mL) and extracted with ether (20 mL). The aqueous layer was collected and acidified to pH ~2 with 1M HCl and extracted with EtOAc (20 mL×3). The combined organic layers were washed with brine, dried over Na₂SO₄, filtered and concentrated to afford N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(spiro[3.5]nonan-7-ylmethyl)-L-threonine (50 mg, 36%) as a white solid. LCMS m/z=674.4 [M+H]⁺; ¹H NMR (400 MHz, DMSO-d6) δ 8.41-8.27 (m, 2H), 7.86-7.77 (m, 1H), 7.39-7.22 (m, 5H), 5.35 (s, 2H), 4.45-4.38 (m, 1H), 4.26-4.06 (m, 2H), 3.99-3.87 (m, 3H), 3.77-3.72 (m, 1H), 3.67-3.54 (m, 2H), 3.50-3.43 (m, 1H), 3.28-3.25 (m, 1H), 3.08-3.02 (m, 1H), 1.83-1.75 (m, 2H), 1.71-1.61 (m, 6H), 1.59-1.52 (m, 2H), 1.41-1.28 (m, 2H), 1.23-1.00 (m, 12H), 0.90-0.81 (m, 3H), 0.69-0.63 (m, 1H).

### Synthesis of ethyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-difluoropropanoate (I-47)

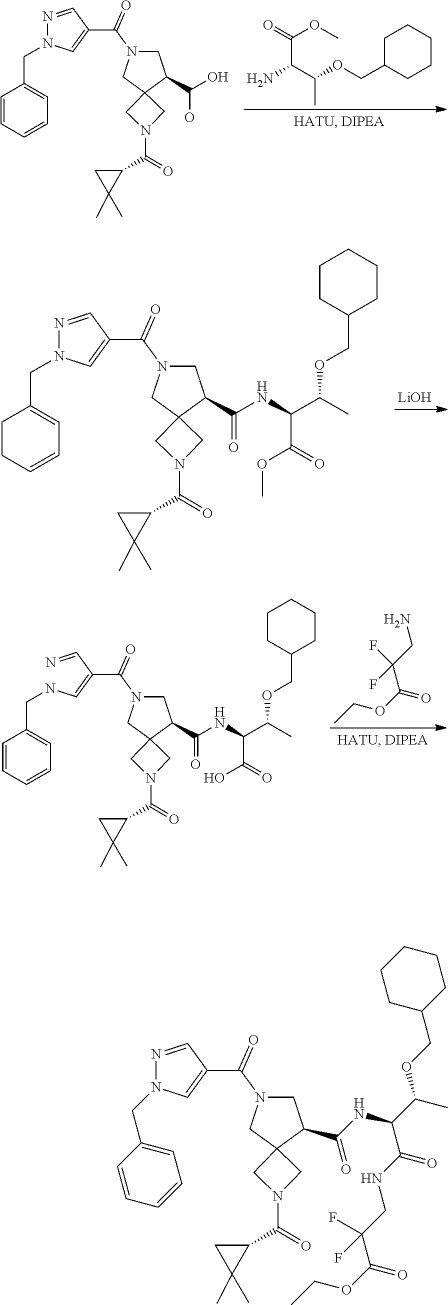

Step 1: methyl N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threoninate: To a solution of (S)-6-(1-benzyl-1H-pyrazole-4-carbonyl)-2-((S)-2,2-dimethylcyclopropane-1-carbonyl)-2,6-diazaspiro[3.4]octane-8-carboxylic acid (914 mg, 2.1 mmol) in DCM (5 mL) was added HATU (955 mg, 2.51 mmol) and DIPEA (1.08 g, 8.37 mmol) and the mixture stirred at room temperature for 30 min. methyl O-(cyclohexylmethyl)-L-threoninate (480 mg, 2.1 mmol) was added and stirring continued for 2 h. The mixture was diluted with water (30 mL) and extracted with DCM (30 mL×2). The combined organic layers were washed with brine, dried over Na₂SO₄, filtered and concentrated. The residue was purified by prep-TLC (eluent: DCM:MeOH=20:1, v/v) to afford methyl N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threoninate (900 mg, 66%) as a yellow solid. LCMS m/z=648.3 [M+H]⁺; 1H NMR (400 MHz, DMSO-d6) δ 8.40-8.33 (m, 1H), 8.17 (s, 1H), 7.83 (d, J=13.0 Hz, 1H), 7.37-7.22 (m, 5H), 5.35 (s, 2H), 4.52 (d, J=8.6 Hz, 1H), 4.12-4.04 (m, 1H), 3.99-3.87 (m, 3H), 3.74 (dd, J=20.8, 13.2 Hz, 2H), 3.66-3.59 (m, 5H), 3.28 (d, J=9.0 Hz, 1H), 3.13 (m, 2H), 3.07-3.01 (m, 1H), 1.65 (d, J=12.4 Hz, 5H), 1.39 (d, J=29.8 Hz, 2H), 1.20-1.10 (m, 4H), 1.06 (m, 8H), 0.85 (m, 3H), 0.67 (m, 1H).

Step 2: N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonine: To a solution of methyl N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-0-(cyclohexylmethyl)-L-threoninate (850 mg, 1.31 mmol) in a mixture of THF (8 mL), MeOH (2 mL) and H₂O (2 mL) was added LiOH (110 mg, 2.62 mmol). The reaction was stirred at room temperature for 3 h then was diluted with water (50 mL) and extracted with EtOAc (30 mL×2). The aqueous phase was acidified to pH ~2 with 1M HCl and extracted with EtOAc (50 mL×3). The combined organic layers were washed with brine, dried over Na₂SO₄ filtered and concentrated to afford crude (610 mg, 73%) as a yellow solid. LCMS m/z=634.3 [M+H]⁺; ¹H NMR (400 MHz, DMSO-d₆) δ 8.39-8.34 (m, 1H), 7.82 (d, J=13.2 Hz, 1H), 7.37-7.23 (m, 5H), 5.35 (s, 2H), 4.40 (s, 1H), 4.14 (m, 2H), 3.99-3.83 (m, 3H), 3.72 (m, 2H), 3.60 (d, J=9.2 Hz, 1H), 3.51-3.43 (m, 1H), 3.32 (s, 3H), 3.08 (t, J=7.8 Hz, 1H), 1.73-1.58 (m, 5H), 1.45 (m, 1H), 1.32 (d, J=23.6 Hz, 1H), 1.19-1.10 (m, 4H), 1.09-1.02 (m, 8H), 0.84 (d, J=10.2 Hz, 3H), 0.69-0.63 (m, 1H).

Step 3: ethyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-difluoropropanoate: To a solution of N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonine (180 mg, 0.28 mmol) in DMF (3 mL) at −20° C. was added ethyl 3-amino-2,2-difluoropropanoate (59 mg, 0.31 mmol) TEA (144 mg, 1.42 mmol) and T₃P (271 mg, 0.43 mmol) was added. The reaction mixture was allowed to warm to room temperature and was stirred for 30 min, then was heated at 100° C. for 3 h. The reaction was diluted with water (30 mL) and extracted with EtOAc (30 mL×2). The combined organic layers were washed with brine, dried over Na₂SO₄, filtered and concentrated. The residue was purified by prep-TLC (eluent: DCM:MeOH=15:1, v/v) to afford ethyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-difluoropropanoate (159 mg, 73%) as a white solid. LCMS m/z=768.4 [M+H]⁺; ¹H NMR (400 MHz, DMSO-d₆) δ 8.39-8.19 (m, 3H), 7.82 (d, J=17.2 Hz, 1H), 7.37-7.23 (m, 5H), 5.35 (s, 2H), 4.42 (m, 1H), 4.22 (m, 3H), 4.15-3.80 (m, 6H), 3.74 (d, J=13.6 Hz, 4H), 3.61 (s, 1H), 3.16 (dd, J=13.6, 7.0 Hz, 2H), 1.63 (m, 5H), 1.40 (d, J=32.4 Hz, 2H), 1.21 (m, 3H), 1.16-1.01 (m, 12H), 0.85 (m, 3H), 0.66 (d, J=7.6 Hz, 1H).

Table I-3: The compounds listed in Table I-3 were synthesized according to the procedures outlined for I-47 using the appropriate commercially available reagents and/or intermediates described elsewhere.

TABLE I-3

| Example | | | |
|---|---|---|---|
| Number | Compound | ¹H NMR | LCMS |
| I-42 | 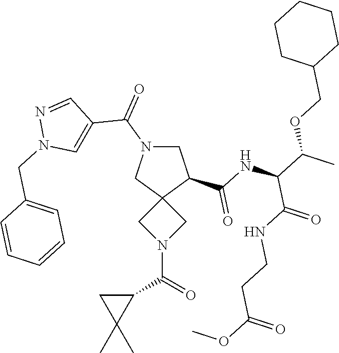 [SMILES: COC(=O)CCNC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1] | ¹H NMR (400 MHz, DMSO-d₆) δ 8.40-8.31 (m, 1H), 8.26-8.12 (m, 1H), 7.99-7.76 (m, 2H), 7.38- 7.23 (m, 5H), 5.35 (s, 2H), 4.31 (dd, J = 10.0, 6.3 Hz, 1H), 4.26-4.02 (m, 2H), 4.01-3.65 (m, 6H), 3.63- 3.55 (m, 4H), 3.53-3.39 (m, 1H), 3.33-3.18 (m, 3H), 3.10 (t, J = 9.3, 6.4 Hz, 1H), 2.47-2.42 (m, 2H), 1.73-1.55 (m, 5H), 1.51- 1.26 (m, 2H), 1.20-0.99 (m, 12H), 0.90-0.76 (m, 3H), 0.72-0.60 (m, 1H). | m/z = 719.5 [M + H]⁺ |
| I-39 | 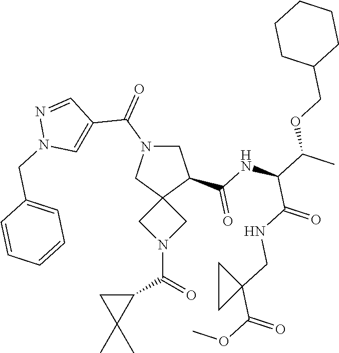 [SMILES: COC(=O)C1(CNC(=O)[C@@H](NC(=O)[C@@H]2CN(C(=O)c3cnn(Cc4ccccc4)c3)CC23CN(C(=O)[C@H]2CC2(C)C)C3)[C@@H](C)OCC2CCCCC2)CC1] | ¹H NMR (400 MHz, DMSO-d6) δ 8.36 (d, J = 15.4 Hz, 1H), 8.23 (dd, J = 25.2, 8.8 Hz, 1H), 7.82 (d, J = 16.0 Hz, 2H), 7.36-7.23 (m, 5H), 5.35 (d, J = 2.8 Hz, 2H), 4.39 (d, J = 8.4 Hz, 1H), 4.14-3.88 (m, 4H), 3.73 (s, 3H), 3.60 (d, J = 3.2 Hz, 4H), 3.20-3.12 (m, 2H), 1.64 (d, J = 14.2 Hz, 5H), 1.35 (s, 3H), 1.22- 0.97 (m, 16H), 0.85 (s, 6H), 0.67 (s, 1H). | m/z = 745.2 [M + H]⁺ |
| I-31 | 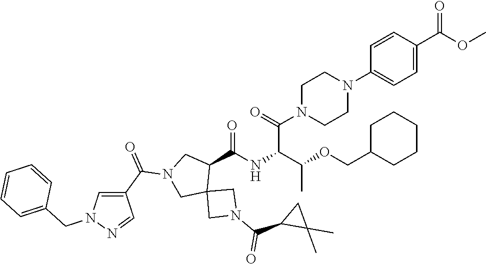 [SMILES: COC(=O)c1ccc(N2CCN(C(=O)[C@@H](NC(=O)[C@@H]3CN(C(=O)c4cnn(Cc5ccccc5)c4)CC34CN(C(=O)[C@H]3CC3(C)C)C4)[C@@H](C)OCC3CCCCC3)CC2)cc1] | ¹H NMR (400 MHz, DMSO-d₆) δ 8.52-8.42 (m, 1H), 8.40-8.30 (m, 1H), 7.86-7.76 (m, 3H), 7.38- 7.21 (m, 5H), 6.97 (d, J = 8.6 Hz, 2H), 5.34 (d, J = 9.4 Hz, 2H), 4.99-4.86 (m, 1H), 4.27-3.84 (m, 5H), 3.82-3.49 (m, 12H), 3.44- 3.35 (m, 2H), 3.26-3.07 (m, 2H), 1.71-1.53 (m, 5H), 1.51-1.27 (m, 2H), 1.18-0.98 (m, 12H), 0.91- 0.77 (m, 3H), 0.70-0.64 (m, 1H). | m/z = 836.4 [M + H]⁺ |
| 1-27 | 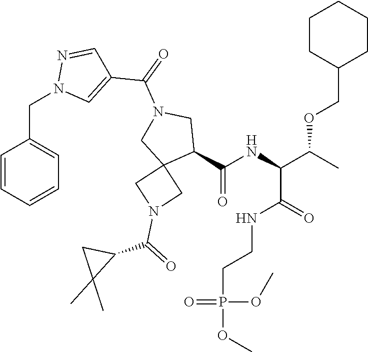 [SMILES: COP(=O)(CCNC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1)OC] | ¹H NMR (400 MHz, DMSO-d₆) δ 8.30 (s, 1H), 8.07 (s, 1H), 7.82 (s, 2H), 7.39-7.22 (m, 5H), 5.36 (s, 2H), 4.36-3.56 (m, 16H), 3.56-3.22 (m, 4H), 3.16- 3.10 (m, 1H), 1.93 (m, 2H), 1.66 (t, J = 12.4 Hz, 5H), 1.46 (s, 1H), 1.32 (d, J = 18.6 Hz, 1H), 1.25-1.02 (m, 12H), 0.87 (d, J = 9.2 Hz, 3H), 0.67 (m, 1H). | m/z = 769.2 [M + H]⁺ |
| I-17 | 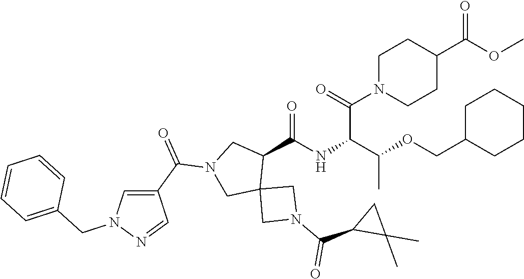 [SMILES: COC(=O)C1CCN(C(=O)[C@@H](NC(=O)[C@@H]2CN(C(=O)c3cnn(Cc4ccccc4)c3)CC23CN(C(=O)[C@H]2CC2(C)C)C3)[C@@H](C)OCC2CCCCC2)CC1] | H NMR (400 MHz, DMSO- d₆) δ 8.45-8.28 (m, 2H), 7.82 (d, J = 15.4 Hz, 1H), 7.42-7.17 (m, 5H), 5.35 (s, 2H), 4.96-4.80 (m, 1H), 4.31-3.35 (m, 15H), 3.25- 3.00 (m, 3H), 1.91-1.78 (m, 2H), 1.71-1.57 (m, 5H), 1.56-1.27 (m, 4H), 1.25-1.01 (m, 13H), 0.92- 0.78 (m, 3H), 0.71-0.64 (m, 1H). | m/z = 759.4 [M + H]⁺ |

### Synthesis of 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)propanoic acid (I-41)

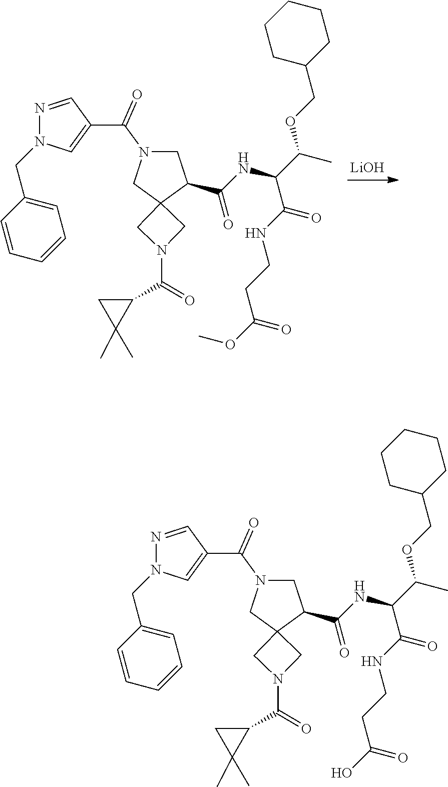

To a solution of methyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)propanoate (70 mg, 0.1 mmol) in a mixture of THF, water and EtOH (2.0 mL/0.5 mL/0.5 mL) was added LiOH (10 mg, 0.25 mmol). The reaction was stirred at room temperature for 2 h then was diluted with water (10 mL) and extracted with ether (30 mL). The aqueous layer was collected, acidified to pH ~2 with 1M HCl and extracted with EtOAc (30 mL×3). The combined organic layers were washed with brine, dried over Na₂SO₄, filtered and concentrated. The residue was purified by prep-HPLC to afford 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)propanoic acid (47 mg, 69%) as a white solid. LCMS m/z=705.4 [M+H]⁺; ¹H NMR (400 MHz, DMSO-d6) δ 8.36 (d, J=13.3 Hz, 1H), 8.25-8.12 (m, 1H), 7.96-7.76 (m, 2H), 7.38-7.22 (m, 5H), 5.35 (s, 2H), 4.36-4.28 (m, 1H), 4.26-4.12 (m, 1H), 4.10-4.03 (m, 1H), 4.00-3.94 (m, 1H), 3.92-3.85 (m, 1H), 3.80-3.66 (m, 3H), 3.64-3.57 (m, 1H), 3.15-3.05 (m, 2H), 2.42-2.34 (m, 3H), 1.71-1.58 (m, 5H), 1.49-1.32 (m, 2H), 1.27-1.23 (m, 1H), 1.19-1.15 (m, 2H), 1.12-1.10 (m, 1H), 1.09-1.07 (m, 2H), 1.06-0.98 (m, 6H), 0.89-0.78 (m, 3H), 0.71-0.63 (m, 1H).

Table I-4: The compounds listed in Table I-4 were synthesized according to the procedures outlined for I-41 using the appropriate commercially available reagents and/or intermediates described elsewhere.

TABLE I-4

| Example | | | |
|---|---|---|---|
| Number | Compound | ¹H NMR | LCMS |
| I-28 | 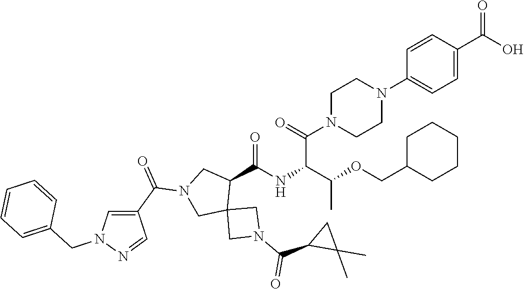 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCN(c2ccc(C(=O)O)cc2)CC1] | ¹H NMR (400 MHz, DMSO-d₆) δ 8.49-8.40 (m, 1H), 8.39- 8.30 (m, 2H), 7.86-7.75 (m, 3H), 7.38-7.21 (m, 5H), 6.94 (d, J = 8.6 Hz, 2H), 5.34 (d, J = 8.8 Hz, 2H), 4.97-4.87 (m, 1H), 4.26-3.81 (m, 6H), 3.79-3.49 (m, 10H), 3.24-3.05 (m, 4H), 1.71-1.27 (m, 7H), 1.20-1.02 (m, 12H), 0.92-0.78 (m, 3H), 0.71-0.64 (m, 1H). | m/z = 822.5 [M + H]⁺ |
| I-46 | 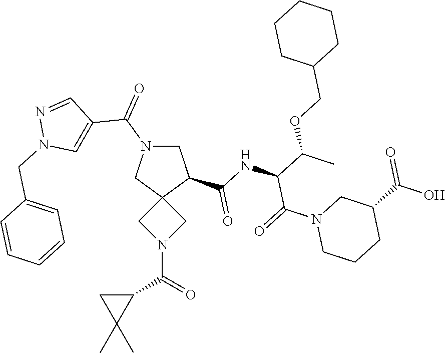 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCC[C@@H](C(=O)O)C1] | ¹H NMR (400 MHz, DMSO-d₆) δ 12.39 (s, 1H), 8.39 (s, 2H), 7.85 (s, 1H), 7.31 (d, J = 31.6 Hz, 5H), 5.35 (d, J = 3.8 Hz, 2H), 4.94 (d, J = 71.0 Hz, 1H), 4.36-3.36 (m, 12H), 3.20 (d, J = 18.2 Hz, 3H), 2.83 (s, 1H), 2.42 (s, 1H), 1.97 (s, 1H), 1.65 (s, 7H), 1.44 (s, 1H), 1.30 (s, 2H), 1.21-1.00 (m, 12H), 0.86 (s, 3H), 0.71-0.65 (m, 1H). | m/z = 743.4 [M + H]⁺ |
| I-45 | 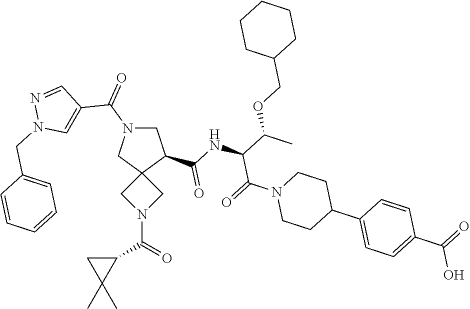 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCC(c2ccc(C(=O)O)cc2)CC1] | ¹H NMR (400 MHz, DMSO-d6) δ 8.37 (s, 2H), 7.85 (q, J = 15.4, 11.7 Hz, 3H), 7.33 (d, J = 7.8 Hz, 7H), 5.35 (s, 2H), 4.94 (s, 1H), 4.55 (s, 1H), 4.17 (d, J = 8.8 Hz, 2H), 4.05 (d, J = 7.4 Hz, 1H), 3.99 (s, 1H), 3.86 (d, J = 10.2 Hz, 1H), 3.74 (s, 1H), 3.62 (s, 3H), 3.19 (s, 4H), 2.88 (s, 1H), 1.85 (s, 1H), 1.64 (d, J = 19.0 Hz, 5H), 1.50-1.27 (m, 3H), 1.08 (d, J = 11.8 Hz, 10H), 0.86 (s, 3H), 0.67 (d, J = 5.0 Hz, 1H). | m/z = 821.4 [M + H]⁺ |

### Synthesis of (S)-6-(1-benzyl-1H-pyrazole-4-carbonyl)-N-((2S,3R)-3-(cyclohexylmethoxy)-1-((3-(methylsulfonamido)-3-oxopropyl)amino)-1-oxobutan-2-yl)-2-((S)-2,2-dimethylcyclopropane-1-carbonyl)-2,6-diazaspiro[3.4]octane-8-carboxamide (1-33)

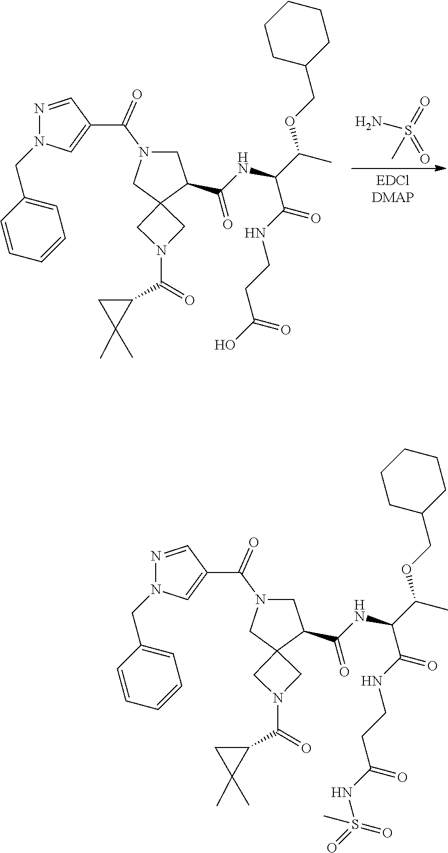

To a solution of 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)propanoic acid (63 mg, 0.09 mmol) in DCM (2 mL) was added methanesulfonamide (10 mg, 0.11 mmol), EDCI (15 mg, 0.076 mmol) and DMAP (2 mg, 0.014 mmol). The reaction mixture stirred at room temperature for 2 h then was diluted with water (30 mL) and extracted with DCM (50 mL×3). The combined organic layers were washed with brine, dried over Na₂SO₄, filtered and concentrated. The residue was purified by prep-HPLC to afford (S)-6-(1-benzyl-1H-pyrazole-4-carbonyl)-N-((2S,3R)-3-(cyclohexylmethoxy)-1-((3-(methylsulfonamido)-3-oxopropyl)amino)-1-oxobutan-2-yl)-2-((S)-2,2-dimethylcyclopropane-1-carbonyl)-2,6-diazaspiro[3.4]octane-8-carboxamide (25.8 mg, 37%) as a white solid. LCMS m/z=780.3 [M−H]⁻; ¹H NMR (400 MHz, DMSO-d6) δ 8.39-8.31 (m, 1H), 8.28-8.12 (m, 1H), 8.07-7.75 (m, 2H), 7.44-7.17 (m, 5H), 5.35 (s, 2H), 4.39-3.82 (m, 6H), 3.81-3.66 (m, 3H), 3.65-3.57 (m, 1H), 3.54-3.36 (m, 1H), 3.30-3.24 (m, 2H), 3.24-3.17 (m, 4H), 3.13 (t, J=8.0 Hz, 1H), 2.48-2.38 (m, 2H), 1.73-1.55 (m, 5H), 1.51-1.26 (m, 2H), 1.21-0.97 (m, 12H), 0.91-0.78 (m, 3H), 0.73-0.61 (m, 1H).

### Synthesis of (5-methyl-2-oxo-1,3-dioxol-4-yl)methyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoate(I-32)

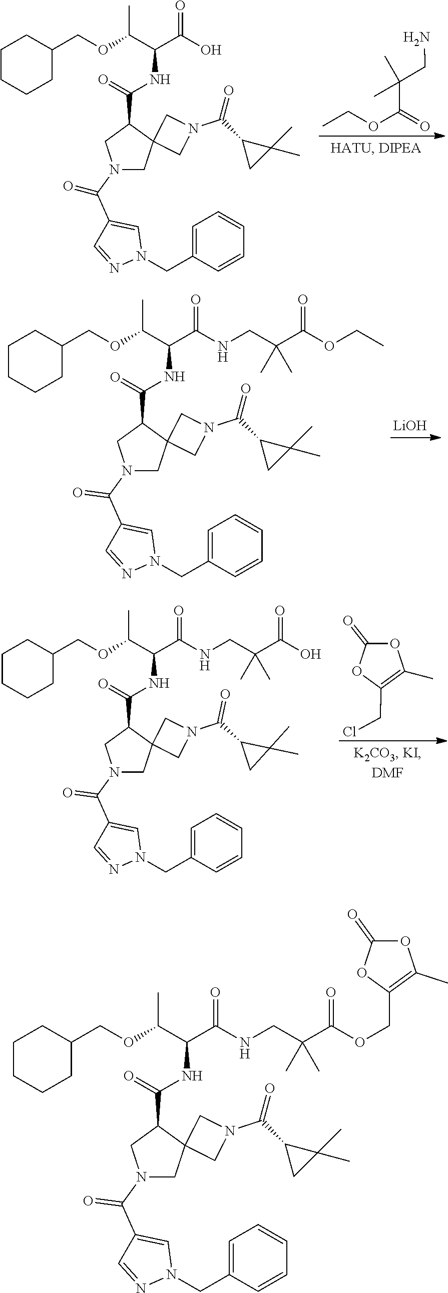

Step 1: ethyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoate: To a solution of N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonine (200 mg, 0.32 mmol) in DCM (4 mL) was added HATU (132 mg, 0.35 mmol) and DIPEA (122 mg, 0.95 mmol) and the mixture stirred at room temperature for 30 min. Ethyl 3-amino-2,2-dimethylpropanoate (46 mg, 0.32 mmol) was added and stirring continued for 2 h. The mixture was diluted with water (30 mL) and extracted with DCM (30 mL×2). The combined organic layers were washed with brine, dried over Na₂SO₄, filtered and concentrated. The residue was purified by prep-TLC (eluent: DCM:MeOH=15:1, v/v) to afford ethyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoate (180 mg, 75%) as a yellow solid. LCMS m/z=761.5 [M+H]⁺; ¹H NMR (400 MHz, DMSO-d₆) δ 8.30 (dd, J=40.8, 17.2 Hz, 2H), 7.82 (d, J=18.2 Hz, 1H), 7.61 (d, J=23.6 Hz, 1H), 7.37-7.23 (m, 5H), 5.35 (s, 2H), 4.42 (s, 1H), 4.26-3.80 (m, 8H), 3.78-3.57 (m, 5H), 3.24-3.08 (m, 3H), 1.68 (m, 5H), 1.35 (m, 1H), 1.17 (d, J=13.6 Hz, 7H), 1.09-1.01 (m, 15H), 0.85 (s, 3H), 0.67 (m, 1H).

Step 2: 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoic acid: To a solution of ethyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoate (100 mg, 0.13 mmol) in a mixture of THF (4 mL), EtOH (1 mL) and H₂O (1 mL) was added LiOH (11 mg, 0.26 mmol). The mixture was stirred at room temperature for 2 h then was diluted with water (50 mL) and extracted with EtOAc (30 mL×2). The aqueous phase was collected, acidified with 1M HCl to pH ~2 extracted with EtOAc and the combined organic layers concentrated. The residue obtained was purified by prep-HPLC to afford 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoic acid (40.7 mg, 42%) as a yellow solid, LCMS m/z=733.3 [M+H]⁺; ¹H NMR (400 MHz, DMSO-d₆) δ 8.40-8.20 (m, 2H), 7.82 (d, J=17.8 Hz, 1H), 7.57 (d, J=22.6 Hz, 1H), 7.37-7.23 (m, 5H), 5.35 (s, 2H), 4.42 (dd, J=8.8, 3.4 Hz, 1H), 4.25-4.10 (m, 1H), 4.07-3.85 (m, 3H), 3.78-3.59 (m, 4H), 3.56-3.40 (m, 2H), 3.17 (m, 4H), 1.67 (dd, J=25.2, 13.2 Hz, 5H), 1.49-1.25 (m, 2H), 1.21 (d, J=18.6 Hz, 4H), 1.09-1.01 (m, 14H), 0.84 (d, J=12.4 Hz, 3H), 0.69-0.64 (m, 1H).

Step 3: (5-methyl-2-oxo-1,3-dioxol-4-yl)methyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoate: To a solution of 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoic acid (45 mg, 0.06 mmol) in DMF (3 mL) was added K₂CO₃ (26 mg, 0.18 mmol), KI (3 mg, 0.02 mmol) and 4-(chloromethyl)-5-methyl-1,3-dioxol-2-one (11 mg, 0.07 mmol). The reaction mixture was heated at 60° C. for 3 h then was diluted with water (30 mL) and extracted with EtOAc (30 mL×2). The combined organic layers were washed with brine, dried over Na₂SO₄, filtered and concentrated. The residue was purified by prep-HPLC to afford (5-methyl-2-oxo-1,3-dioxol-4-yl)methyl 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoate (12.7 mg, 24%) as a white solid. LCMS m/z=845.4 [M+H]⁺; ¹H NMR (400 MHz, DMSO-d₆) δ 8.35 (d, J=19.2 Hz, 1H), 8.19 (dd, J=20.4, 8.2 Hz, 1H), 7.87-7.66 (m, 2H), 7.40-7.20 (m, 5H), 5.35 (s, 2H), 4.91 (d, J=11.4 Hz, 2H), 4.37 (d, J=19.2 Hz, 1H), 4.25-4.03 (m, 2H), 4.00-3.57 (m, 7H), 3.45 (m, 2H), 3.24-3.07 (m, 3H), 2.14 (d, J=5.6 Hz, 3H), 1.64 (m, 5H), 1.39 (m, 2H), 1.18-1.00 (m, 18H), 0.84 (m, 3H), 0.66 (d, J=7.4 Hz, 1H).

### Synthesis of (S)-6-(1-benzyl-1H-pyrazole-4-carbonyl)-N-((2S,3R)-3-(cyclohexylmethoxy)-1-((2,2-dimethyl-3-(methylamino)-3-oxopropyl)amino)-1-oxobutan-2-yl)-2-((S)-2,2-dimethylcyclopropane-1-carbonyl)-2,6-diazaspiro[3.4]octane-8-carboxamide (I-37)

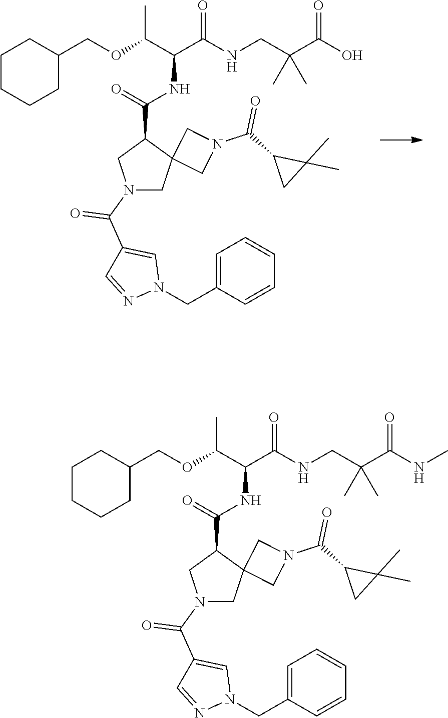 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)NCC(C)(C)C(=O)O ; CNC(=O)C(C)(C)CNC(=O)[C@@H](NC(=O)[C@@H]1CN(C(=O)c2cnn(Cc3ccccc3)c2)CC12CN(C(=O)[C@H]1CC1(C)C)C2)[C@@H](C)OCC1CCCCC1]

To a solution of 3-((2S,3R)-2-((S)-6-(1-benzyl-1H-pyrazole-4-carbonyl)-2-((S)-2,2-dimethylcyclopropane-1-carbonyl)-2,6-diazaspiro[3.4]octane-8-carboxamido)-3-(cyclohexylmethoxy)butanamido)-2,2-dimethylpropanoic acid (50 mg, 0.07 mmol) in DCM (1 mL) was added HATU (40 mg, 0.105 mmol) and DIEA (27 mg, 0.21 mmol) and the mixture stirred for 30 min. Methylamine hydrochloride (6 mg, 0.084 mmol) was added and stirring was continued overnight. The solvent was removed and the residue obtained purified by RP-column to afford (S)-6-(1-benzyl-1H-pyrazole-4-carbonyl)-N-((2S,3R)-3-(cyclohexylmethoxy)-1-((2,2-dimethyl-3-(methylamino)-3-oxopropyl)amino)-1-oxobutan-2-yl)-2-((S)-2,2-dimethylcyclopropane-1-carbonyl)-2,6-diazaspiro[3.4]octane-8-carboxamide (35 mg, 69%) as a white solid. LCMS m/z=746.3 [M+H]⁺; ¹H NMR (400 MHz, CDCl₃) δ 7.93-7.74 (m, 2H), 7.33 (s, 4H), 7.24 (s, 2H), 6.84 (s, 1H), 6.04-5.74 (m, 1H), 5.29 (s, 2H), 4.48-3.81 (m, 10H), 3.47-3.06 (m, 5H), 2.81-2.65 (m, 3H), 1.88 (s, 1H), 1.71 (s, 2H), 1.52 (s, 1H), 1.22-1.04 (m, 19H), 0.96-0.79 (m, 5H), 0.72 (s, 1H).

### Synthesis of (S)-1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-2-carboxylic acid (I-36)

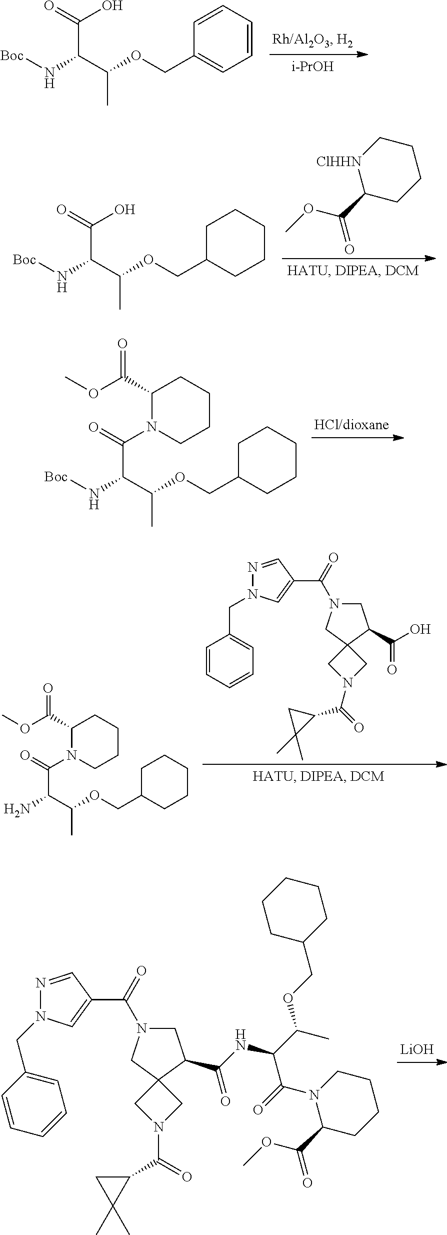

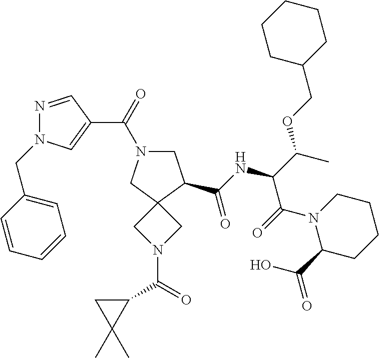 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCCC[C@H]1C(=O)O]

Step 1: N-(tert-butoxycarbonyl)-O-(cyclohexylmethyl)-L-threonine: To a solution of O-benzyl-N-(tert-butoxycarbonyl)-L-threonine (1 g, 3.2 mmol) in i-PrOH (10 mL) was added 40% Rh/Al₂O₃ (400 mg). The reaction was stirred under a H₂ atmosphere for 48 h then the catalyst was removed by filtration through Celite and the filtrate concentrated to afford N-(tert-butoxycarbonyl)-O-(cyclohexylmethyl)-L-threonine (1.0 g, 100%) which was used directly in the next step. LCMS m/z=216.2 [M+H-100]⁺; ¹H NMR (400 MHz, DMSO-d6) δ 6.21 (d, J=9.2 Hz, 1H), 4.33 (d, J=4.2 Hz, 1H), 3.84-3.72 (m, 2H), 3.27-3.21 (m, 1H), 1.68-1.61 (m, 3H), 1.41-1.36 (m, 7H), 1.20-1.10 (m, 2H), 1.09-1.00 (m, 9H), 0.90-0.78 (m, 2H).

Step 2: methyl (S)-1-(N-(tert-butoxycarbonyl)-O-(cyclohexylmethyl)-L-threonyl)piperidine-2-carboxylate: To a solution of N-(tert-butoxycarbonyl)-O-(cyclohexylmethyl)-L-threonine (200 mg, 0.63 mmol) in DCM (3 mL) was added HATU (265 mg, 0.7 mmol) and DIPEA (245 mg, 1.9 mmol) and the mixture was stirred at room temperature for 30 min. methyl (S)-piperidine-2-carboxylate hydrochloride (125 mg, 0.7 mmol) was added and stirring continued for 2 h. The mixture was diluted with water (15 mL) and extracted with DCM (30 mL×3). The combined organic layers were washed with brine, dried over Na₂SO₄, filtered and concentrated. The residue was purified by RP-column (70% MeCN in water) to afford methyl (S)-1-(N-(tert-butoxycarbonyl)-O-(cyclohexylmethyl)-L-threonyl)piperidine-2-carboxylate (210 mg, 75%) as a colorless oil. LCMS m/z=441.2 [M+H]⁺; ¹H NMR (400 MHz, DMSO-d6) δ 6.55-6.37 (m, 1H), 5.22-5.05 (m, 1H), 4.47-4.36 (m, 1H), 4.01 (d, J=13.8 Hz, 1H), 3.65 (s, 3H), 3.60-3.49 (m, 1H), 3.28-3.22 (m, 1H), 3.17-3.04 (m, 2H), 2.23-1.94 (m, 1H), 1.72-1.47 (m, 9H), 1.37 (d, J=6.8 Hz, 9H), 1.33-1.26 (m, 1H), 1.23-1.09 (m, 4H), 1.08-1.00 (m, 3H), 0.91-0.81 (m, 2H).

Step 3: methyl (S)-1-(O-(cyclohexylmethyl)-L-threonyl)piperidine-2-carboxylate: To a solution of methyl (S)-1-(N-(tert-butoxycarbonyl)-O-(cyclohexylmethyl)-L-threonyl)piperidine-2-carboxylate (50 mg, 0.11 mmol) in DCM (1 mL) was added HCl (4M in dioxane, 0.3 mL). The reaction was stirred for 3.5 h then the solvent was removed under reduced pressure to afford methyl (S)-1-(O-(cyclohexylmethyl)-L-threonyl)piperidine-2-carboxylate (38 mg, quant.) LCMS m/z=341.2 [M+H]⁺.

Step 4: methyl (S)-1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-2-carboxylate: To a solution of (S)-6-(1-benzyl-1H-pyrazole-4-carbonyl)-2-((S)-2,2-dimethylcyclopropane-1-carbonyl)-2,6-diazaspiro[3.4]octane-8-carboxylic acid (140 mg, 0.32 mmol) in DCM (2 mL) was added HATU (134 mg, 0.35 mmol) and DIPEA (124 mg, 0.92 mmol) and the mixture stirred for 30 min. Methyl (S)-1-(O-(cyclohexylmethyl)-L-threonyl)piperidine-2-carboxylate (120 mg, 0.35 mmol) was added and stirring continued for 2 h. The mixture was diluted with water (20 mL) and extracted with DCM (50 mL×2). The combined organic layers were washed with brine, dried over Na₂SO₄, filtered and concentrated. The residue was purified by RP-column (72% MeCN in water) to afford methyl (S)-1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-2-carboxylate (19.5 mg, 8%) as a white solid. LCMS m/z=759.3 [M+H]⁺; ¹H NMR (400 MHz, DMSO-d6) δ 8.43-8.33 (m, 2H), 7.85-7.78 (m, 1H), 7.37-7.23 (m, 5H), 5.35 (s, 2H), 5.24-5.05 (m, 1H), 4.91-4.84 (m, 1H), 4.36-3.53 (m, 14H), 3.21 (d, J=6.4 Hz, 3H), 1.71-1.57 (m, 7H), 1.48-1.02 (m, 18H), 0.85 (s, 3H), 0.67 (s, 1H).

Step 5: (S)-1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-2-carboxylic acid: To a solution of methyl (S)-1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-2-carboxylate (41 mg, 0.54 mmol) in a mixture of THF (0.8 mL), MeOH (0.2 mL) and water (0.2 mL) was added LiOH (12 mg, 0.27 mmol). The mixture was stirred at room temperature for 3 h then was diluted with water and ether. The aqueous phase was acidified to pH ~2 with 1M HCl, extracted with EtOAc and concentrated. The residue obtained was purified by prep-HPLC to afford (S)-1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-2-carboxylic acid (56.9 mg, 100%) as a white solid. LCMS m/z=745.3 [M+H]⁺; ¹H NMR (400 MHz, DMSO-d6) δ 8.40-8.31 (m, 2H), 7.85-7.79 (m, 1H), 7.38-7.23 (m, 5H), 5.35 (s, 2H), 5.15-5.08 (m, 1H), 4.93-4.84 (m, 1H), 4.26-4.09 (m, 1H), 4.06-3.50 (m, 10H), 3.22-3.14 (m, 2H), 2.20-2.02 (m, 1H), 1.76-1.52 (m, 8H), 1.49-1.22 (m, 5H), 1.17-1.01 (m, 12H), 0.89-0.79 (m, 3H), 0.70-0.64 (m, 1H).

Table I-5: The compounds listed in Table I-5 were synthesized according to the procedures outlined for I-36 using the appropriate commercially available reagents and/or intermediates described elsewhere.

TABLE I-5

| Example | | | |
|---|---|---|---|
| Number | Compound | ¹H NMR | LCMS |
| I-35 | 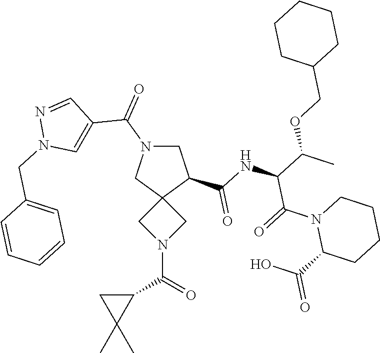 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)N1CCCC[C@@H]1C(=O)O] | ¹H NMR (400 MHz, DMSO-d₆) δ 8.54-8.21 (m, 2H), 7.82 (d, J = 16.8 Hz, 1H), 7.38-7.22 (m, 5H), 5.35 (s, 2H), 4.98 (s, 1H), 4.33-3.52 (m, 14H), 2.16 (d, J = 13.2 Hz, 1H), 1.64 (d, J = 11.6 Hz, 7H), 1.49-1.25 (m, 5H), 1.20-0.98 (m, 13H), 0.85 (d, J = 6.6 Hz, 3H), 0.67 (d, J = 4.0 Hz, 1H). | m/z = 745.4 [M + H]⁺ |

### Synthesis of (S)-6-(1-benzyl-1H-pyrazole-4-carbonyl)-N-((2S,3R)-3-(cyclohexylmethoxy)-1-((2,4-difluorophenyl)sulfonamido)-1-oxobutan-2-yl)-2-((S)-2,2-dimethylcyclopropane-1-carbonyl)-2,6-diazaspiro[3.4]octane-8-carboxamide (I-26)

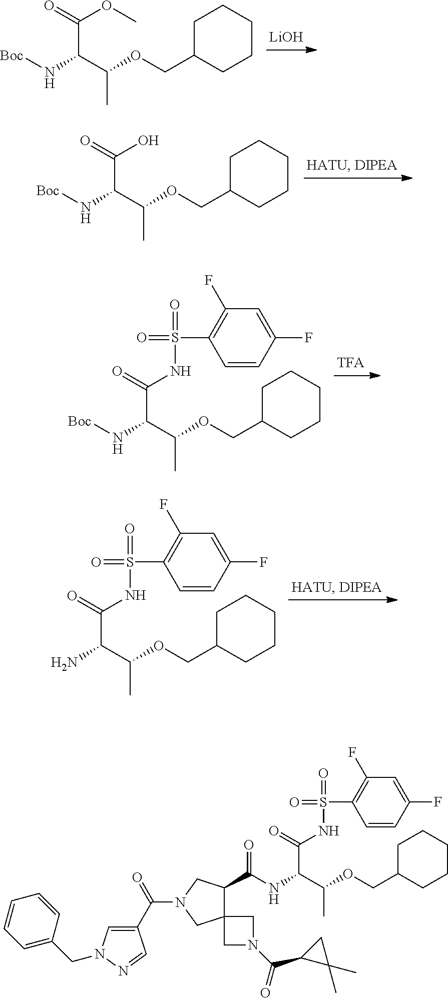

Step 1: tert-butyl ((2S,3R)-3-(cyclohexylmethoxy)-1-((2,4-difluorophenyl)sulfonamido)-1-oxobutan-2-yl)carbamate: To a solution of methyl N-(tert-butoxycarbonyl)-O-(cyclohexylmethyl)-L-threoninate (500 mg, 1.52 mmol) in a mixture of THF (8 mL), MeOH (2 mL) and water (2 mL) was added LiOH (110 mg, 4.56 mmol). The mixture was stirred at room temperature for 2 h then was diluted with water (50 mL) and extracted with EtOAc (30 mL×2). The aqueous phase was acidified to pH ~2 with 1M HCl and extracted with EtOAc (50 mL×3). The combined organic layers were washed with brine, dried over Na₂SO₄ filtered and concentrated to afford tert-butyl ((2S,3R)-3-(cyclohexylmethoxy)-1-((2,4-difluorophenyl)sulfonamido)-1-oxobutan-2-yl)carbamate (450 mg, 94%) as a yellow oil. LCMS m/z=314.2 [M−H]⁻; ¹H NMR (400 MHz, DMSO-d6) δ 6.17 (d, J=9.4 Hz, 1H), 3.98 (dd, J=9.4, 3.4 Hz, 1H), 3.86-3.76 (m, 1H), 3.25 (td, J=6.6, 3.1 Hz, 1H), 3.07 (s, 1H), 2.33-1.89 (m, 1H), 1.74-1.32 (m, 14H), 1.26-1.10 (m, 3H), 1.07 (d, J=6.2 Hz, 3H), 0.85 (q, J=11.4, 10.8 Hz, 2H).

Step 2: N-(tert-butoxycarbonyl)-O-(cyclohexylmethyl)-L-threonine: To a solution of N-(tert-butoxycarbonyl)-O-(cyclohexylmethyl)-L-threonine (400 mg, 1.27 mmol) in DCM (8 mL) was added HATU (724 mg, 1.9 mmol) and DIPEA (655 mg, 5.08 mmol) and the mixture stirred at room temperature for 30 min. 2,4-difluorobenzenesulfonamide (193 mg, 1.27 mmol) was added and stirring continued for 2 h. The solvent was removed under vacuum and the residue purified by prep-TLC (DCM:MeOH=20:1, v/v) to afford N-(tert-butoxycarbonyl)-O-(cyclohexylmethyl)-L-threonine (300 mg, 50%) as yellow oil. LCMS m/z=491.2 [M+H]⁺; ¹H NMR (400 MHz, DMSO-d6) δ 7.90 (s, 1H), 7.38 (s, 1H), 7.20 (s, 1H), 3.82 (s, 1H), 3.69 (d, J=6.8 Hz, 1H), 3.08 (t, J=7.78 Hz, 1H), 2.87 (t, J=7.8 Hz, 1H), 2.12 (s, 1H), 1.58 (s, 5H), 1.36 (s, 9H), 1.31-1.22 (m, 3H), 1.09 (d, J=9.2 Hz, 3H), 0.98 (d, J=6.2 Hz, 3H).

Step 3: (2S,3R)-2-amino-3-(cyclohexylmethoxy)-N-((2,4-difluorophenyl)sulfonyl)butanamide: To a solution of N-(tert-butoxycarbonyl)-O-(cyclohexylmethyl)-L-threonine (20 mg, 0.041 mmol) in DCM (2 mL) was added TFA (0.5 mL) and the reaction stirred at room temperature for 2 h. The solvent was removed under vacuum to afford tert-butyl ((2S,3R)-3-(cyclohexylmethoxy)-1-((2,4-difluorophenyl)sulfonamido)-1-oxobutan-2-yl)carbamate (16 mg, 100%) which was used directly in the next step. LCMS m/z=391.1 [M+H]⁺.

Step 4: (S)-6-(1-benzyl-1H-pyrazole-4-carbonyl)-N-((2S,3R)-3-(cyclohexylmethoxy)-1-((2,4-difluorophenyl)sulfonamido)-1-oxobutan-2-yl)-2-((S)-2,2-dimethylcyclopropane-1-carbonyl)-2,6-diazaspiro[3.4]octane-8-carboxamide: To a solution of tert-butyl ((2S,3R)-3-(cyclohexylmethoxy)-1-((2,4-difluorophenyl)sulfonamido)-1-oxobutan-2-yl)carbamate (61 mg, 0.14 mmol) in DCM (3 mL) was added HATU (80 mg, 0.21 mmol) and DIPEA (72 mg, 0.56 mmol) and the mixture stirred at room temperature for 30 min. (2S,3R)-1-(4-(1H-imidazol-5-yl)piperidin-1-yl)-2-amino-3-(cyclohexylmethoxy)butan-1-one (62 mg, 0.14 mmol) was added and stirring continued for 2 h. The solvent was removed under vacuum and the residue purified by prep-TLC (DCM:MeOH=20:1, v/v) to afford (S)-6-(1-benzyl-1H-pyrazole-4-carbonyl)-N-((2S,3R)-3-(cyclohexylmethoxy)-1-((2,4-difluorophenyl)sulfonamido)-1-oxobutan-2-yl)-2-((S)-2,2-dimethylcyclopropane-1-carbonyl)-2,6-diazaspiro[3.4]octane-8-carboxamide (26 mg, 23%) as a white solid. LCMS m/z=809.2 [M+H]⁺; ¹H NMR (400 MHz, DMSO-d6) δ 8.36 (s, 2H), 8.07-7.94 (m, 1H), 7.82 (d, J=15.8 Hz, 1H), 7.59 (s, 1H), 7.34 (d, J=7.4 Hz, 6H), 5.35 (s, 2H), 4.40 (d, J=9.6 Hz, 1H), 4.28-3.50 (m, 10H), 3.13 (t, J=7.8 Hz, 1H), 2.82 (s, 1H), 1.59 (s, 5H), 1.29 (d, J=33.6 Hz, 2H), 1.15-0.98 (m, 12H), 0.84 (s, 1H), 0.78-0.51 (m, 3H).

Table I-6: The compounds listed in Table I-6 were synthesized according to the procedures outlined for I-26 using the appropriate commercially available reagents and/or intermediates described elsewhere.

TABLE I-6

| Example | | | |
|---|---|---|---|
| Number | Compound | ¹H NMR | LCMS |
| 1-18 | 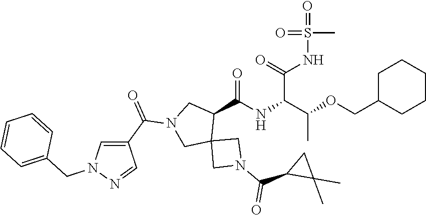 [SMILES: C[C@@H](OCC1CCCCC1)[C@H](NC(=O)[C@@H]1CN(C(=O)c2cnn(Cc3ccccc3)c2)CC12CN(C(=O)[C@H]1CC1(C)C)C2)C(=O)NS(C)(=O)=O] | ¹H NMR (400 MHz, DMSO-d₆) δ 11.83 (s, 1H), 8.35 (s, 2H), 7.81 (s, 1H), 7.33 (s, 5H), 5.35 (s, 2H), 4.47 (s, 1H), 4.07 (s, 2H), 3.98 (s, 3H), 3.67 (d, J = 48.0 Hz, 4H), 3.45 (s, 1H), 3.20 (s, 4H), 3.11 (d, J = 7.8 Hz, 1H), 1.62 (s, 5H), 1.44 (s, 2H), 1.04 (s, 12H), 0.85 (t, J = 6.4 Hz, 3H), 0.66 (d, J = 6.2 Hz, 1H). | m/z = 711.3 [M + H]⁺ |
| I-22 | 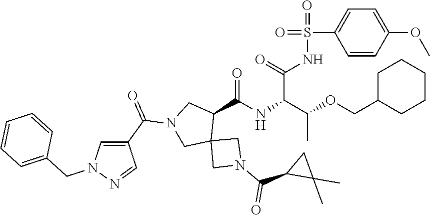 [SMILES: COc1ccc(S(=O)(=O)NC(=O)[C@@H](NC(=O)[C@@H]2CN(C(=O)c3cnn(Cc4ccccc4)c3)CC23CN(C(=O)[C@H]2CC2(C)C)C3)[C@@H](C)OCC2CCCCC2)cc1] | ¹H NMR (400 MHz, Chloroform-d) δ 8.78 (d, J = 12.4 Hz, 1H), 8.42 (d, J = 6.6 Hz, 1H), 7.77 (d, J = 11.0 Hz, 1H), 7.60 (s, 1H), 7.43 (d, J = 8.4 Hz, 1H), 7.32 (s, 2H), 6.90 (s, 2H), 5.40-5.31 (m, 2H), 4.39-4.01 (m, 7H), 3.80 (d, J = 2.6 Hz, 5H), 2.14 (s, 2H), 1.68 (s, 1H). | m/z = 803.3 [M + H]⁺ |

### Synthesis of (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2-(1-(trifluoromethyl)cyclopropane-1-carbonyl)-2,6-diazaspiro[3.4]octane-8-carboxamide (1-34)

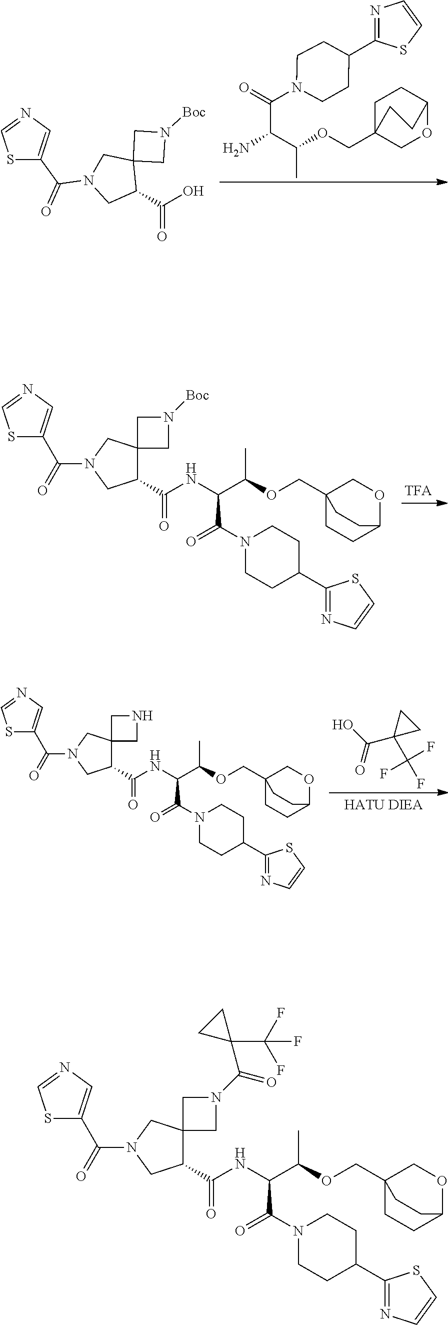 [SMILES: CC(C)(C)OC(=O)N1CC2(C1)CN(C(=O)c1cncs1)C[C@H]2C(=O)O ; C[C@@H](OCC12CCC(CC1)OC2)[C@H](N)C(=O)N1CCC(c2nccs2)CC1 ; C[C@@H](OCC12CCC(CC1)OC2)[C@H](NC(=O)[C@@H]1CN(C(=O)c2cncs2)CC12CN(C(=O)OC(C)(C)C)C2)C(=O)N1CCC(c2nccs2)CC1 ; C[C@@H](OCC12CCC(CC1)OC2)[C@H](NC(=O)[C@@H]1CN(C(=O)c2cncs2)CC12CNC2)C(=O)N1CCC(c2nccs2)CC1 ; O=C(O)C1(C(F)(F)F)CC1 ; C[C@@H](OCC12CCC(CC1)OC2)[C@H](NC(=O)[C@@H]1CN(C(=O)c2cncs2)CC12CN(C(=O)C1(C(F)(F)F)CC1)C2)C(=O)N1CCC(c2nccs2)CC1]

Step 1: tert-butyl (S)-8-(((2S,3R)-3-((2-oxabicyclo[2.2.2]octan-4-yl)methoxy)-1-oxo-1-(4-(thiazol-2-yl)piperidin-1-yl)butan-2-yl)carbamoyl)-6-(thiazole-5-carbonyl)-2,6-diazaspiro[3.4]octane-2-carboxylate: To a solution of (S)-2-(tert-butoxycarbonyl)-6-(thiazole-5-carbonyl)-2,6-diazaspiro[3.4]octane-8-carboxylic acid (206 mg, 0.56 mmol) in DCM (4 mL) was added HATU (213 mg, 0.56 mmol) and DIPEA (217 mg, 1.68 mmol) and the mixture stirred at room temperature for 30 min. (2S,3R)-3-((2-oxabicyclo[2.2.2]octan-4-yl)methoxy)-2-amino-1-(4-(thiazol-2-yl)piperidin-1-yl)butan-1-one (220 mg, 0.56 mmol) was added and stirring continued for another 2 h. The mixture was diluted with water (30 mL) and extracted with DCM (50 mL×3). The combined organic layers were washed with brine, dried over Na₂SO₄, filtered and concentrated. The residue obtained was purified by RP-column to afford tert-butyl (S)-8-(((2S,3R)-3-((2-oxabicyclo[2.2.2]octan-4-yl)methoxy)-1-oxo-1-(4-(thiazol-2-yl)piperidin-1-yl)butan-2-yl)carbamoyl)-6-(thiazole-5-carbonyl)-2,6-diazaspiro[3.4]octane-2-carboxylate (170 mg, 45%) as a white solid. LCMS m/z=743.2 [M+H]⁺; ¹H NMR (400 MHz, DMSO-d₆) δ 9.31-9.20 (m, 1H), 8.36 (dd, J=20.2, 12.3 Hz, 2H), 7.76-7.08 (m, 3H), 4.96-4.12 (m, 2H), 4.11-3.70 (m, 10H), 3.67-3.54 (m, 3H), 3.51-3.34 (m, 2H), 3.27-2.70 (m, 2H), 2.25 (s, 1H), 2.14-1.78 (m, 3H), 1.73-1.46 (m, 4H), 1.37 (d, J=5.4 Hz, 9H), 1.34 (s, 3H), 1.24 (s, 1H), 1.17 (t, J=7.1 Hz, 1H), 1.04 (s, 2H).

Step 2: (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-oxo-1-(4-(thiazol-2-yl)piperidin-1-yl)butan-2-yl)carbamoyl)-6-(thiazole-5-carbonyl)-2,6-diazaspiro[3.4]octane-2-carboxylate (75 mg, 0.11 mmol) in DCM (2 mL) was added TFA (1 mL) and the reaction stirred at room temperature for 2 h. The solvent was removed under vacuum and the residue obtained purified by prep-HPLC to afford (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2,6-diazaspiro[3.4]octane-8-carboxamide (8.2 mg, 13%) as a white solid. LCMS m/z=643.3[M+H]⁺; ¹H NMR (400 MHz, Methanol-d₄) δ 9.21-9.12 (m, 1H), 8.52 (s, 1H), 8.40-8.31 (m, 1H), 7.76-7.65 (m, 1H), 7.52-7.40 (m, 1H), 4.38-3.68 (m, 12H), 3.64-3.33 (m, 3H), 3.28-2.80 (m, 2H), 2.27-1.75 (m, 5H), 1.75-1.57 (m, 5H), 1.56-1.40 (m, 2H), 1.26-1.13 (m, 3H).

Step 3: (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2-(1-(trifluoromethyl)cyclopropane-1-carbonyl)-2,6-diazaspiro[3.4]octane-8-carboxamide: To a solution of 1-(trifluoromethyl)cyclopropane-1-carboxylic acid (15 mg, 0.1 mmol) in DCM (2 mL) was added HATU (38 mg, 0.1 mmol) and DIPEA (51 mg, 0.4 mmol) and the mixture stirred at room temperature for 30 min. (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2,6-diazaspiro[3.4]octane-8-carboxamide (64 mg, 0.1 mmol) was added and stirring continued for 2 h. The mixture was diluted with water (30 mL) and extracted with DCM (50 mL×3). The combined organic layers were washed with brine, dried over Na₂SO₄, filtered and concentrated. The residue was purified by prep-HPLC to afford (S)—N-((2S,3R)-3-((2-oxabicyclo[2.2.2]octan-4-yl)methoxy)-1-oxo-1-(4-(thiazol-2-yl)piperidin-1-yl)butan-2-yl)-6-(thiazole-5-carbonyl)-2-(1-(trifluoromethyl)cyclopropane-1-carbonyl)-2,6-diazaspiro[3.4]octane-8-carboxamide (30.7 mg, 40%) as a white solid. LCMS m/z=779.2 [M+H]⁺; ¹H NMR (400 MHz, DMSO-d₆) δ 9.24 (s, 1H), 8.40-8.32 (m, 2H), 7.72 (s, 1H), 7.61 (s, 1H), 4.91 (s, 1H), 4.45-4.19 (m, 3H), 4.08 (s, 3H), 3.81 (s, 2H), 3.64 (s, 3H), 3.54 (s, 2H), 3.45 (s, 1H), 3.17-3.07 (m, 1H), 3.04-2.96 (m, 1H), 2.88-2.70 (m, 1H), 2.07 (s, 2H), 1.82 (s, 2H), 1.59-1.47 (m, 6H), 1.43-1.21 (m, 5H), 1.19-1.11 (m, 3H), 1.02 (s, 4H).

### Synthesis of (S)—N-((2S,3R)-3-((2-oxabicyclo[2.2.2]octan-4-yl)methoxy)-1-(4-cyanopiperidin-1-yl)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (I-25)

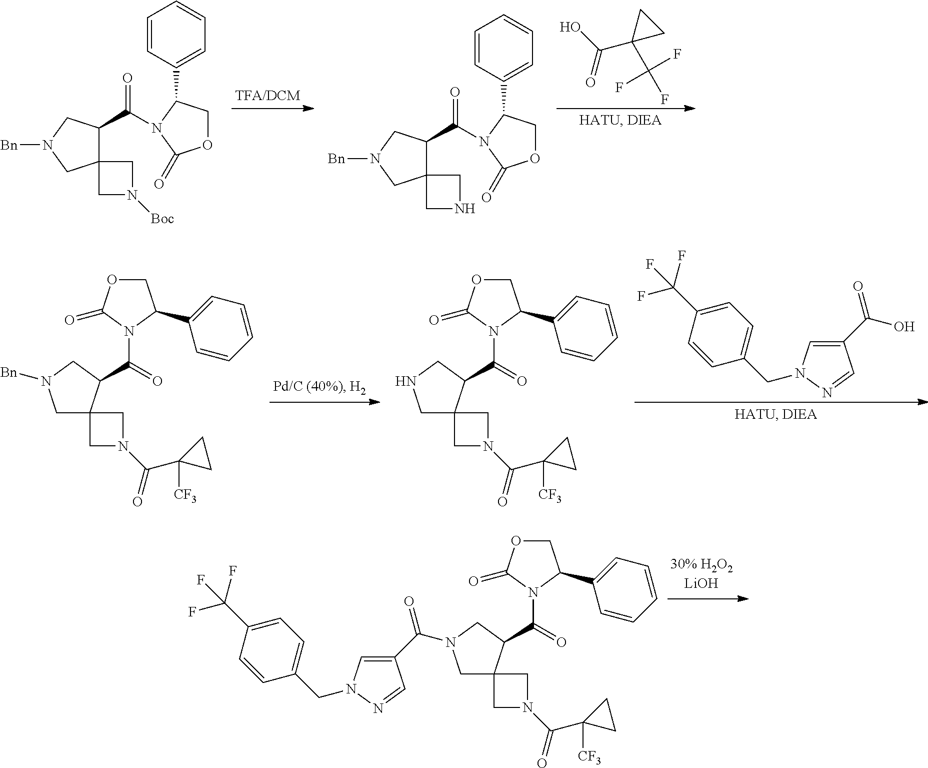

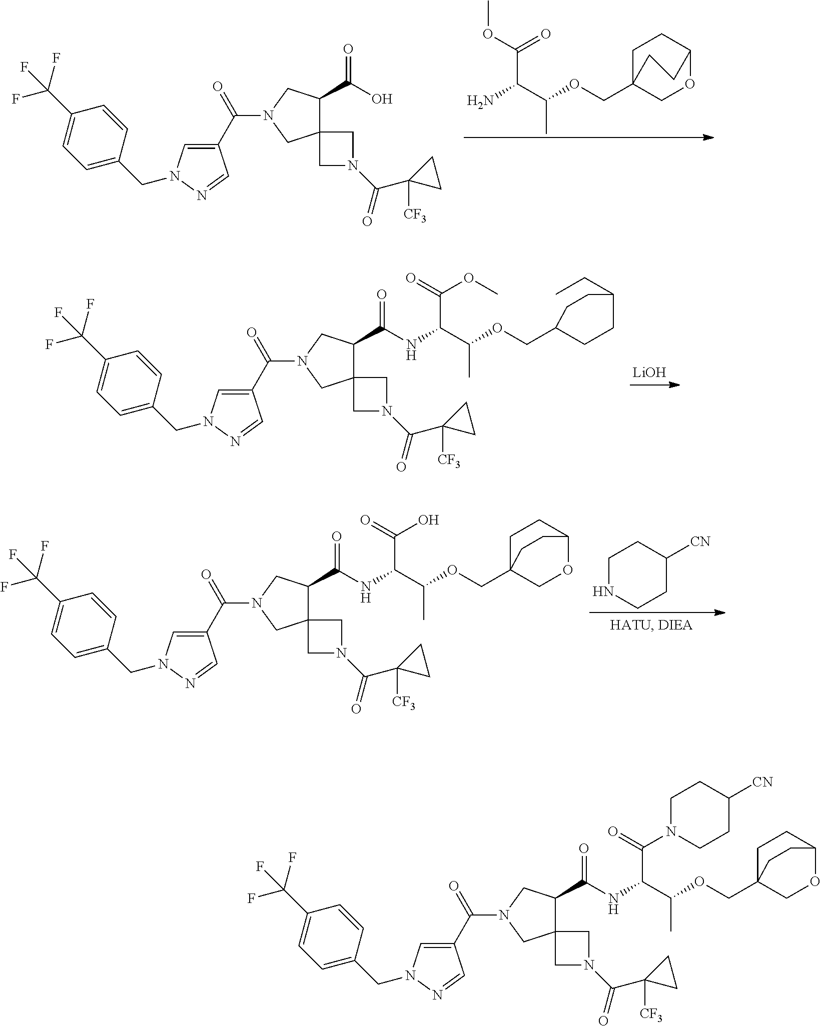

Step 1: (R)-3-((S)-6-benzyl-2,6-diazaspiro[3.4]octane-8-carbonyl)-4-phenyloxazolidin-2-one: To a solution of tert-butyl (S)-6-benzyl-8-((R)-2-oxo-4-phenyloxazolidine-3-carbonyl)-2,6-diazaspiro[3.4]octane-2-carboxylate (30 g, 61 mmol) in DCM (200 mL) was added TFA (100 mL) and the reaction stirred at room temperature for 2 h. The solvent was removed under vacuum and the residue used directly for the next step. LCMS m/z=392.1 [M+H]⁺.

Step 2: (R)-3-((S)-6-benzyl-2-(1-(trifluoromethyl)cyclopropane-1-carbonyl)-2,6-diazaspiro[3.4]octane-8-carbonyl)-4-phenyloxazolidin-2-one: To a solution of (R)-3-((S)-6-benzyl-2,6-diazaspiro[3.4]octane-8-carbonyl)-4-phenyloxazolidin-2-one (23.89 g, 0.061 mol) in DCM (200 mL) was added HATU (23.2 g, 0.061 mol) and the reaction mixture stirred at room temperature for 30 min. 1-(trifluoromethyl)cyclopropane-1-carboxylic acid (9.4 g, 0.061 mol) and DIPEA (31.5 g, 0.224 mol) were added and the reaction stirred for another 3 h. The mixture was quenched with water and extracted with DCM (80 mL×3). The combined organic phases were washed with 2 M HCl for three times, dried over Na₂SO₄ filtered and concentrated. The residue obtained was purified by silica gel column (DCM:MeOH=60:1, v/v) to afford (R)-3-((S)-6-benzyl-2-(1-(trifluoromethyl)cyclopropane-1-carbonyl)-2,6-diazaspiro[3.4]octane-8-carbonyl)-4-phenyloxazolidin-2-one (17.8 g, 55%) as a yellow solid. LCMS m/z=528.2 [M+H]⁺; ¹H NMR (400 MHz, DMSO-d₆) δ 7.31-7.44 (m, 10H), 5.51 (dd, J=8.8 Hz, 1H), 3.64-4.25 (m, 8H), 3.39 (s, 5H), 1.20-1.28 (m, 2H), 1.17 (s, 2H).

Step 3: (R)-4-phenyl-3-((S)-2-(1-(trifluoromethyl)cyclopropane-1-carbonyl)-2,6-diazaspiro[3.4]octane-8-carbonyl)oxazolidin-2-one: To a solution of (R)-3-((S)-6-benzyl-2-(1-(trifluoromethyl)cyclopropane-1-carbonyl)-2,6-diazaspiro[3.4]octane-8-carbonyl)-4-phenyloxazolidin-2-one (1 g, 1.9 mmol) in EtOAc (8 mL) was added Pd/C (400 mg, 3.76 mmol) and the reaction mixture stirred at 45° C. overnight under a H₂ atmosphere. The catalyst was removed by filtration through Celite and the filtrate concentrated to afford (R)-4-phenyl-3-((S)-2-(1-(trifluoromethyl)cyclopropane-1-carbonyl)-2,6-diazaspiro[3.4]octane-8-carbonyl)oxazolidin-2-one (800 mg, 97%) as a white solid. LCMS m/z=438.1 [M+H]⁺.

Step 4: (R)-4-phenyl-3-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)oxazolidin-2-one: To a solution of (R)-4-phenyl-3-((S)-2-(1-(trifluoromethyl)cyclopropane-1-carbonyl)-2,6-diazaspiro[3.4]octane-8-carbonyl)oxazolidin-2-one (5.4 g, 12.35 mmol) in DCM (50 mL) was added HATU (4.7 g, 12.35 mmol) and the reaction mixture stirred at room temperature for 30 min. 1-(4-(trifluoromethyl)benzyl)-1H-pyrazole-4-carboxylic acid (3.7 g, 13.58 mmol) and DIPEA (6.4 g, 49.38 mmol) were added and the reaction stirred for another 3 h. The mixture was quenched with water and extracted with DCM (50 mL×4). The combined organic phases were washed with brine, dried over Na₂SO₄ filtered and concentrated under vacuum. The residue obtained was purified by silica gel column (DCM:MeOH=80:1, v/v) to afford (R)-4-phenyl-3-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)oxazolidin-2-one (4.94 g, 58%) as a yellow solid. LCMS m/z=690.1 [M+H]⁺; ¹H NMR (400 MHz, DMSO-d₆) δ 8.37 (d, J=9.2 Hz, 2H), 7.81 (d, J=14.0 Hz, 2H), 7.71 (d, J=7.9 Hz, 3H), 7.48-7.29 (m, 7H), 7.25-7.22 (m, 4H), 7.10 (s, 2H), 5.52-5.44 (m, 5H), 4.77 (t, J=8.8 Hz, 2H), 4.43-4.07 (m, 10H), 1.24 (s, 4H), 1.16 (s, 3H).

Step 5: (S)-6-(1-(4-(trifluoromethyl)benzyl)-1H-pyrazole-4-carbonyl)-2-(1-(trifluoromethyl) cyclopropane-1-carbonyl)-2,6-diazaspiro[3.4]octane-8-carboxylic acid: To a solution of (R)-4-phenyl-3-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)oxazolidin-2-one (5.3 g, 7.68 mmol) in a mixture of THF (40 mL) and water (10 mL) at 0° C. was added 30% H₂O₂ (520 mg, 15.36 mmol) and LiOH (968 mg, 23.04 mmol). The reaction was stirred at 0° C. for 1 h then was quenched with water and extracted with EtOAc (50 mL×2). The aqueous phase was collected, the pH adjusted to 2-3 with 1M HCl and extracted with EtOAc (50 mL×3). The combined organic layers were washed with brine, dried over Na₂SO₄ filtered and concentrated under vacuum to afford (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (2.39 g, 57%) as a white solid. LCMS m/z=545.2 [M+H]⁺; ¹H NMR (400 MHz, DMSO-d₆) δ 12.69 (s, 1H), 8.42 (d, J=6.4 Hz, 1H), 7.88-7.84 (m, 1H), 7.72 (d, J=8.0 Hz, 2H), 7.44 (d, J=8.0 Hz, 2H), 5.49 (s, 2H), 4.43-3.98 (m, 5H), 3.91-3.65 (m, 3H), 3.28-3.27 (m, 1H), 1.23-1.09 (m, 4H).

Step 6: methyl O-((2-oxabicyclo[2.2.2]octan-4-yl)methyl)-N—((S)-6-(1-(4-(trifluoromethyl) benzyl)-1H-pyrazole-4-carbonyl)-2-(1-(trifluoromethyl)cyclopropane-1-carbonyl)-2,6-diazaspiro[3.4]octane-8-carbonyl)-L-threoninate: To a solution of methyl 0-((2-oxabicyclo[2.2.2]octan-4-yl)methyl)-L-threoninate (1.28 g, 2.35 mmol) in DCM (15 mL) was added HATU (895 mg, 2.35 mmol) and the mixture stirred at room temperature for 30 min. (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (606 mg, 2.35 mmol) and DIPEA (1.22 g, 9.4 mmol) were added and the reaction stirred for another 3 h. The mixture was quenched with water and extracted with EtOAc (50 mL×3). The combined organic phases were washed with brine, dried over Na₂SO₄ filtered and concentrated under vacuum. The residue obtained was purified by silica gel column (DCM:MeOH=30:1, v/v) to afford methyl O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threoninate (1.04 g, 57%) as a white solid. LCMS m/z=784.2 [M+H]⁺; ¹H NMR (400 MHz, DMSO-d₆) δ 8.43 (d, J=7.0 Hz, 1H), 7.86 (dd, J₁=13.2, J₂=5.9 Hz, 1H), 7.72 (d, J=8.0 Hz, 2H), 7.44 (d, J=7.8 Hz, 2H), 7.11 (d, J=8.2 Hz, 1H), 5.48 (s, 2H), 4.57-4.53 (m, 1H), 4.22 (s, 1H), 4.08-3.72 (m, 6H), 3.66-3.34 (m, 11H), 2.25 (s, 1H), 1.58-1.12 (m, 12H), 1.04-1.00 (m, 2H).

Step 7: O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonine: To a solution of methyl O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threoninate (500 mg, 0.638 mmol) in a mixture of THE (4 mL), MeOH (1 mL) and water (1 mL) at 0° C. was added LiOH (80 mg, 1.914 mmol). The mixture was stirred at room temperature for 2 h. The reaction was diluted with ether and the pH adjusted to 2-3 with 1M HCl. The aqueous was extracted with EtOAc and the combined organic layers concentrated under vacuum. The residue obtained was purified by RP-column (22% MeCN in water) to afford O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonine (200 mg, 41%) as a white solid. LCMS m/z=770.1 [M+H]⁺; ¹H NMR (400 MHz, DMSO-d₆) δ 8.42 (s, 1H), 8.26 (d, J=9.2 Hz, 1H), 7.86 (d, J=13.2 Hz, 1H), 7.72 (d, J=8.1 Hz, 2H), 7.44 (d, J=7.9 Hz, 2H), 5.48 (s, 2H), 4.43-4.35 (m, 1H), 3.95-3.75 (m, 4H), 3.65-3.61 (m, 2H), 3.57 (s, 1H), 3.51-3.45 (m, 5H), 3.42-3.37 (m, 3H), 1.82 (s, 1H), 1.58-1.43 (m, 5H), 1.39-1.27 (m, 5H), 1.15 (s, 2H), 1.02-0.98 (m, 2H).

Step 8: (S)—N-((2S,3R)-3-((2-oxabicyclo[2.2.2]octan-4-yl)methoxy)-1-(4-cyanopiperidin-1-yl)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide: To a solution of piperidine-4-carbonitrile (13 mg, 0.118 mmol) in DCM (2 mL) was added DIPEA (61 mg, 0.472 mmol), O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonine (90 mg, 0.118 mmol) and HATU (45 mg, 0.118 mmol) and the reaction was stirred at room temperature for 2 h. The reaction was quenched with water and extracted with DCM (15 mL×3). The combined organic phases were washed with brine, dried over Na₂SO₄ filtered and concentrated under vacuum. The residue obtained was purified by prep-HPLC to afford (S)—N-((2S,3R)-3-((2-oxabicyclo[2.2.2]octan-4-yl)methoxy)-1-(4-cyanopiperidin-1-yl)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (9.9 mg, 10%) as a white solid. LCMS m/z=862.2 [M+H]⁺; ¹H NMR (400 MHz, DMSO-d₆) δ 8.45-8.28 (m, 2H), 7.85 (d, J=15.6 Hz, 1H), 7.72 (d, J=8.0 Hz, 2H), 7.44 (d, J=7.7 Hz, 2H), 5.48 (s, 2H), 4.87 (s, 1H), 4.21-3.73 (m, 8H), 3.65-3.35 (m, 8H), 3.12-2.99 (m, 4H), 1.89-1.51 (m, 10H), 1.37 (s, 2H), 1.23-1.16 (m, 4H), 1.01 (s, 3H).

Table I-7: The compound listed in Table I-7 were synthesized according to the procedures outlined for I-25 using the appropriate commercially available reagents and/or intermediates described elsewhere.

TABLE I-7

| Example | | | |
|---|---|---|---|
| Number | Compound | ¹H NMR | LCMS |
| I-16 | 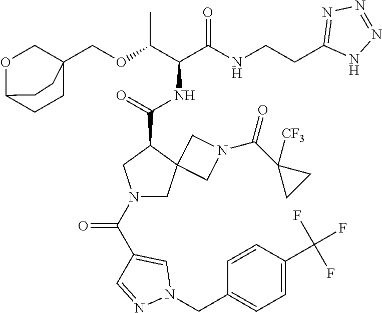 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)NCCc1nnn[nH]1] | ¹H NMR (400 MHz, Chloroform-d) δ 8.20- 7.78 (m, 2H), 7.60 (s, 2H), 7.48-7.31 (m, 3H), 5.38 (s, 2H), 4.56-3.90 (m, 10H), 3.87-3.53 (m, 6H), 3.43-3.09 (m, 4H), 1.77 (d, J = 153.8 Hz, 6H), 1.42-1.02 (m, 9H). | m/z = 865.2 [M + H]⁺ |

### Synthesis of 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetic acid (I-6)

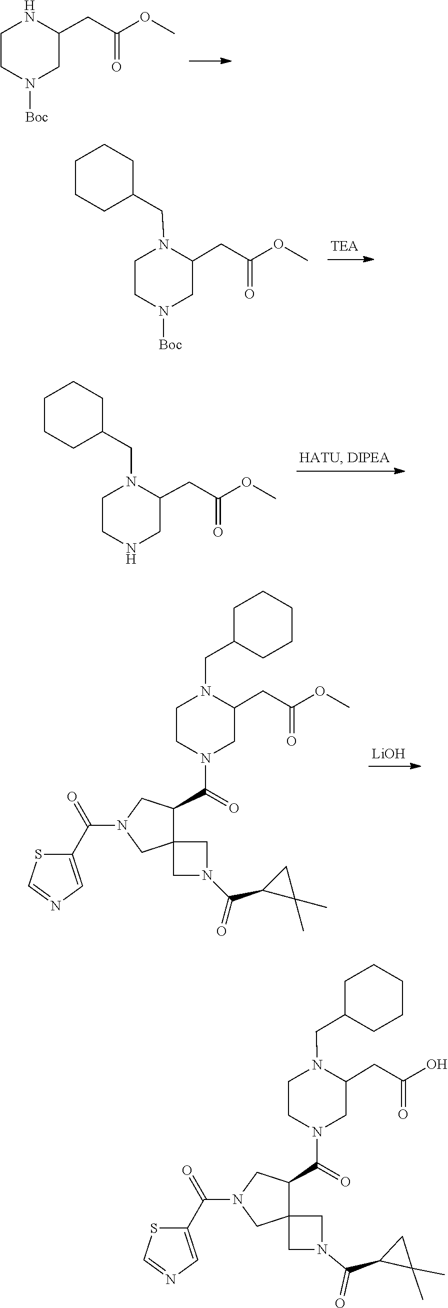 [SMILES: COC(=O)CC1CN(C(=O)OC(C)(C)C)CCN1 ; COC(=O)CC1CN(C(=O)OC(C)(C)C)CCN1CC1CCCCC1 ; COC(=O)CC1CNCCN1CC1CCCCC1 ; COC(=O)CC1CN(C(=O)[C@@H]2CN(C(=O)c3cncs3)CC23CN(C(=O)[C@H]2CC2(C)C)C3)CCN1CC1CCCCC1 ; "LiOH" ; CC1(C)C[C@@H]1C(=O)N1CC2(CN(C(=O)c3cncs3)C[C@H]2C(=O)N2CCN(CC3CCCCC3)C(CC(=O)O)C2)C1]

Step 1: tert-butyl 4-(cyclohexylmethyl)-3-(2-methoxy-2-oxoethyl)piperazine-1-carboxylate: To a solution of tert-butyl 3-(2-methoxy-2-oxoethyl)piperazine-1-carboxylate (250 mg, 1 mmol) and cyclohexanecarbaldehyde (112 mg, 1.32 mmol) in DCE (4 mL) was added sodium triacetoxyborohydride (318 mg, 1.5 mmol). The reaction was stirred at room temperature for 5 h then was diluted with water (30 mL) and extracted with DCM (30 mL×2). The combined organic layers were washed with brine, dried over Na₂SO₄, filtered and concentrated to afford tert-butyl 4-(cyclohexylmethyl)-3-(2-methoxy-2-oxoethyl)piperazine-1-carboxylate (240 mg, 91%) as a yellow oil. ¹H NMR (400 MHz, DMSO-d6) δ 3.60 (s, 3H), 3.19 (dd, J=13.2, 7.9 Hz, 1H), 3.01 (dd, J=9.6, 6.5 Hz, 1H), 2.86 (d, J=8.0 Hz, 1H), 2.45 (d, J=4.0 Hz, 1H), 2.35-2.20 (m, 4H), 1.81 (d, J=4.8 Hz, 3H), 1.37 (s, 9H), 1.29 (td, J=8.8, 8.1, 2.3 Hz, 6H), 1.22-1.09 (m, 4H), 0.83 (d, J=12.4 Hz, 1H).

Step 2: methyl 2-(1-(cyclohexylmethyl)piperazin-2-yl)acetate: To a solution of tert-butyl 4-(cyclohexylmethyl)-3-(2-methoxy-2-oxoethyl)piperazine-1-carboxylate (350 mg, 1 mmol) in DCM (4 mL) was added TFA (1 mL) and the reaction stirred at room temperature for 2 h. The solvent was removed under vacuum to afford 1-((1-benzyl-1H-pyrazol-4-yl)methyl)-5-(methylcarbamoyl)-1H-indole-3-carboxylic acid (250 mg, 100%) which was used directly in the next step. LCMS m/z=255.2 [M+H]⁺.

Step 3: methyl 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetate: To a solution of (S)-2-((S)-2,2-dimethylcyclopropane-1-carbonyl)-6-(thiazole-5-carbonyl)-2,6-diazaspiro[3.4]octane-8-carboxylic acid (29 mg, 0.08 mmol) in DCM (2 mL) was added HATU (45 mg, 0.12 mmol) and DIPEA (41.28 mg, 0.32 mmol) and the mixture stirred at room temperature for 30 min. Methyl 2-(1-(cyclohexylmethyl)piperazin-2-yl)acetate (20 mg, 0.08 mmol) was added and stirring continued for 2 h. The solvent was removed under vacuum and the residue was purified by prep-HPLC to afford methyl 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetate (14 mg, 29%) as a white solid. LCMS m/z=600.2[M+H]⁺; ¹H NMR (400 MHz, DMSO-d6) δ 9.25 (s, 1H), 8.39 (d, J=5.2 Hz, 1H), 4.11 (dd, J=29.4, 9.2 Hz, 4H), 3.81 (s, 5H), 3.62 (t, J=6.0 Hz, 6H), 2.89 (d, J=16.4 Hz, 2H), 2.58 (s, 1H), 2.41-2.21 (m, 3H), 2.06 (s, 1H), 1.63 (s, 5H), 1.35 (d, J=36.4 Hz, 2H), 1.10 (d, J=8.6 Hz, 6H), 1.05 (q, J=5.6, 5.2 Hz, 3H), 0.86 (s, 3H), 0.70-0.64 (m, 1H).

Step 4: 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetic acid: To a solution of methyl 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetate (250 mg, 0.42 mmol) in a mixture of THF (2 mL), MeOH (0.5 mL) and H₂O (0.5 mL) was added LiOH (30 mg, 1.26 mmol). The mixture was stirred at room temperature for 2 h then was diluted with water (50 mL) and extracted with EtOAc (30 mL×2). The aqueous phase was collected, acidified with 1M HCl to pH ~2 and extracted with EtOAc (50 mL×3). The combined organic layers were washed with brine, dried over Na₂SO₄ and concentrated. The residue was purified by prep-HPLC to afford 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetic acid (40 mg, 16%) as a white solid. LCMS m/z=586.2 [M+H]⁺; ¹H NMR (400 MHz, DMSO-d6) δ 9.25 (s, 1H), 8.37 (d, J=18.8 Hz, 1H), 4.14 (s, 4H), 3.82 (d, J=10.8 Hz, 4H), 3.67 (s, 3H), 3.29 (s, 2H), 2.37 (s, 1H), 2.08 (s, 1H), 1.74 (s, 1H), 1.64 (s, 4H), 1.40 (s, 2H), 1.16 (s, 3H), 1.11 (s, 4H), 1.06-1.02 (m, 3H), 0.85 (s, 3H), 0.67 (s, 1H).

### Synthesis of (S)—N-((2S,3R)-1-(4-(1H-tetrazol-5-yl)piperidin-1-yl)-3-((2-oxabicyclo[2.2.2]octan-4-yl)methoxy)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (I-15)

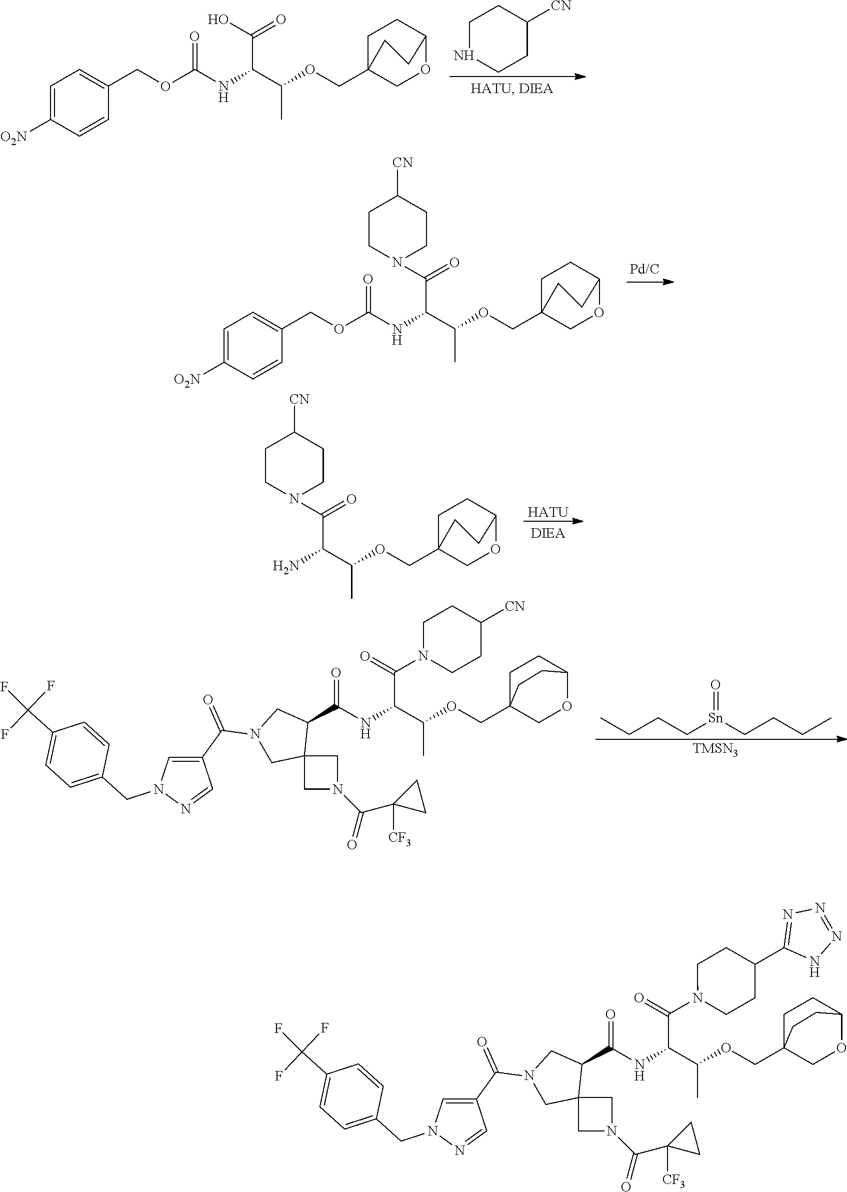

Step 1: 4-nitrobenzyl ((2S,3R)-3-((2-oxabicyclo[2.2.2]octan-4-yl)methoxy)-1-(4-cyanopiperidin-1-yl)-1-oxobutan-2-yl)carbamate: To a solution of piperidine-4-carbonitrile (42 mg, 0.378 mmol) in DCM (2 mL) was added DIPEA (196 mg, 1.512 mmol), O-((2-oxabicyclo[2.2.2]octan-4-yl)methyl)-N-(((4-nitrobenzyl)oxy)carbonyl)-L-threonine (160 mg, 0.378 mmol) and HATU (144 mg, 0.378 mmol) and the reaction stirred for 2 h. The reaction was quenched with water and extracted with DCM (20 mL×3). The combined organic layers were washed with brine, dried over Na₂SO₄ filtered and concentrated under vacuum. The residue obtained was purified by prep-TLC (DCM/MeOH=20/1, v/v) to afford 4-nitrobenzyl ((2S,3R)-3-((2-oxabicyclo[2.2.2]octan-4-yl)methoxy)-1-(4-cyanopiperidin-1-yl)-1-oxobutan-2-yl)carbamate (84 mg, 43%) as a yellow oil. LCMS m/z=515 [M+H]⁺; ¹H NMR (400 MHz, DMSO-d₆) & 8.23 (d, J=6.8 Hz, 2H), 7.62 (d, J=8.4 Hz, 2H), 7.50 (s, 1H), 5.19 (s, 2H), 4.46 (s, 1H), 3.86-3.75 (m, 2H), 3.62-3.50 (m, 3H), 3.48-3.35 (m, 4H), 3.12 (s, 2H), 1.83 (s, 3H), 1.57-1.45 (m, 7H), 1.34-1.32 (m, 2H), 1.03-1.01 (m, 3H).

Step 2: 1-(O-((2-oxabicyclo[2.2.2]octan-4-yl)methyl)-L-threonyl)piperidine-4-carbonitrile: To a solution of 4-nitrobenzyl ((2S,3R)-3-((2-oxabicyclo[2.2.2]octan-4-yl)methoxy)-1-(4-cyanopiperidin-1-yl)-1-oxobutan-2-yl)carbamate (84 mg, 0.16 mmol) in MeOH (2 mL) was added Pd/C (33 mg, 0.31 mmol) and the reaction mixture heated at 50° C. for 2 h under a H₂ atmosphere. The catalyst was removed by filtration through Celite and the filtrate concentrated to afford 1-(O-((2-oxabicyclo[2.2.2]octan-4-yl)methyl)-L-threonyl)piperidine-4-carbonitrile (54 mg, 100%) as a colorless oil. LCMS m/z=336.2 [M+H]⁺.

Step 3: (S)—N-((2S,3R)-3-((2-oxabicyclo[2.2.2]octan-4-yl)methoxy)-1-(4-cyanopiperidin-1-yl)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide: To a solution of 1-(O-((2-oxabicyclo[2.2.2]octan-4-yl)methyl)-L-threonyl)piperidine-4-carbonitrile (54 mg, 0.16 mmol) in DCM (2 mL) was added HATU (62 mg, 0.16 mmol) and the mixture for 30 min. (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (87 mg, 0.16 mmol) and DIPEA (83 mg, 0.64 mmol) were added and stirring continued for 3 h. The mixture was quenched with water and extracted with DCM (20 mL×3). The combined organic layers were washed with brine, dried over Na₂SO₄ filtered and concentrated. The residue obtained was purified by prep-TLC (DCM:MeOH=20:1, v/v) to afford (S)—N-((2S,3R)-3-((2-oxabicyclo[2.2.2]octan-4-yl)methoxy)-1-(4-cyanopiperidin-1-yl)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (80 mg, 58%) as a white solid. LCMS m/z=862.3 [M+H]⁺; ¹H NMR (400 MHz, DMSO-d₆) & 8.42-8.32 (m, 2H), 7.85 (d, J=15.6 Hz, 1H), 7.72 (d, J=8 Hz, 2H), 7.44 (d, J=9.2 Hz, 2H), 5.48 (s, 2H), 4.21-3.36 (m, 19H), 3.24-3.12 (m, 2H), 1.84-1.58 (m, 4H), 1.56-1.43 (m, 5H), 1.38-1.28 (m, 4H), 1.16-1.01 (m, 6H).

Step 4: (S)—N-((2S,3R)-1-(4-(1H-tetrazol-5-yl)piperidin-1-yl)-3-((2-oxabicyclo[2.2.2]octan-4-yl)methoxy)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide: To a solution of (S)—N-((2S,3R)-3-((2-oxabicyclo[2.2.2]octan-4-yl)methoxy)-1-(4-cyanopiperidin-1-yl)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (40 mg, 0.0464 mmol) in Toluene (1 mL) was added dibutyltin oxide (2.3 mg, 0.009 mmol) and TMSN₃ (11 mg, 0.093 mmol). The mixture was heated at 100° C. overnight then the reaction was quenched with water and extracted with EtOAc (15 mL×3). The combined organic phases were washed with brine, dried over Na₂SO₄ filtered and concentrated and the residue obtained purified by prep-HPLC to afford (S)—N-((2S,3R)-1-(4-(1H-tetrazol-5-yl)piperidin-1-yl)-3-((2-oxabicyclo[2.2.2]octan-4-yl)methoxy)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (5.1 mg, 6%) as a light yellow solid. LCMS m/z=905.3 [M+H]⁺; ¹H NMR (400 MHz, DMSO-d₆) & 8.42-8.34 (m, 2H), 7.85 (d, J=14.8 Hz, 1H), 7.72 (d, J=8 Hz, 2H), 7.44 (d, J=8 Hz, 2H), 5.48 (s, 2H), 4.89 (s, 1H), 4.30-4.20 (m, 3H), 4.00-3.87 (m, 5H), 3.76-3.54 (m, 11H), 3.14-2.88 (m, 4H), 2.07-1.99 (m, 2H), 1.70-1.37 (m, 9H), 1.16-1.03 (m, 6H).

### Synthesis of 2-((4-((S)-8-(((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (I-14)

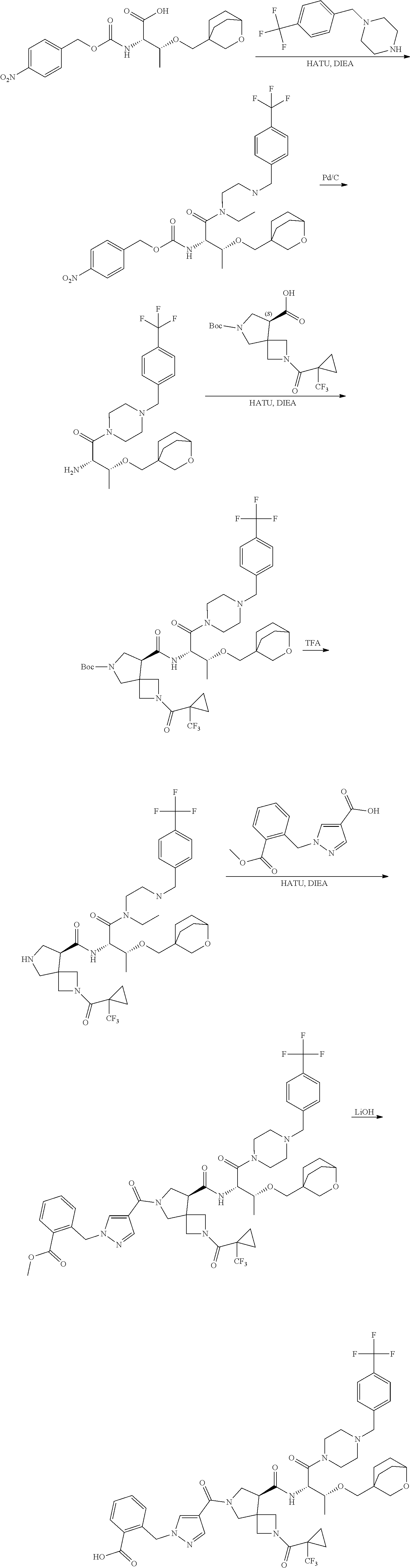

Step 1: 4-nitrobenzyl ((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamate: To a solution of O-((2-oxabicyclo[2.2.2]octan-4-yl)methyl)-N-(((4-nitrobenzyl)oxy)carbonyl)-L-threonine (380 mg, 0.9 mmol) in DCM (6 mL) were added DIPEA (465 mg, 0.36 mmol), 1-(4-(trifluoromethyl)benzyl)piperazine (220 mg, 0.9 mmol) and HATU (342 mg, 0.9 mmol) and the reaction stirred at room temperature for 2 h. The mixture was quenched with water and extracted with DCM (20 mL×3). The combined organic phases were washed with brine, dried over Na₂SO₄ filtered and concentrated. The residue obtained was purified by silica gel column (DCM:MeOH=100:1, v/v) to afford 4-nitrobenzyl ((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamate (440 mg, 73%) as a yellow oil. LCMS m/z=649.2 [M+H]⁺; ¹H NMR (400 MHz, DMSO-d₆) & 8.23 (d, J=8.4 Hz, 2H), 7.68 (s, 2H), 7.62 (d, J=8.4 Hz, 2H), 7.55 (s, 2H), 7.43 (t, J=8.4 Hz, 1H), 5.17 (s, 2H), 4.46 (s, 1H), 3.58-3.57 (m, 5H), 3.50-3.46 (m, 4H), 3.41-3.35 (m, 3H), 2.38-2.32 (m, 4H), 1.50-1.43 (m, 4H), 1.36-1.24 (m, 4H), 1.02 (t, J=6.8 Hz, 3H).

Step 2: (2S,3R)-3-((2-oxabicyclo[2.2.2]octan-4-yl)methoxy)-2-amino-1-(4-(4-(trifluoromethyl) benzyl)piperazin-1-yl)butan-1-one: To a solution of 4-nitrobenzyl ((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamate (200 mg, 0.3083 mmol) in MeOH (2 mL) was added Pd/C (80 mg, 0.7517 mmol) and the reaction heated at 50° C. for 2 h under a hydrogen atmosphere. The catalyst was removed by filtration through Celite and the filtrate concentrated to afford (2S,3R)-3-((2-oxabicyclo[2.2.2]octan-4-yl)methoxy)-2-amino-1-(4-(4-(trifluoromethyl)benzyl) piperazin-1-yl)butan-1-one (144 mg, 100%) as a colorless oil. LCMS m/z=470.25 [M+H]⁺.

Step 3: tert-butyl (S)-8-(((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamoyl)-2-(1-(trifluoromethyl)cyclopropane-1-carbonyl)-2,6-diazaspiro[3.4]octane-6-carboxylate: To a solution of (2S,3R)-3-((2-oxabicyclo[2.2.2]octan-4-yl)methoxy)-2-amino-1-(4-(4-(trifluoromethyl)benzyl)piperazin-1-yl)butan-1-one in DCM (2 mL) was added HATU (116 mg, 0.3 mmol) and the reaction mixture stirred at room temperature for 30 min. (S)-6-(tert-butoxycarbonyl)-2-(1-(trifluoromethyl)cyclopropane-1-carbonyl)-2,6-diazaspiro[3.4]octane-8-carboxylic acid (120 mg, 0.3058 mmol) and DIPEA (158 mg, 1.2 mmol) were added stirring continued for 3 h. The mixture was concentrated under vacuum and the residue purified by RP-column (72% ACN in water) to afford tert-butyl (S)-8-(((2S,3R)-3-((2-oxabicyclo[2.2.2]octan-4-yl)methoxy)-1-oxo-1-(4-(4-(trifluoromethyl) benzyl)piperazin-1-yl)butan-2-yl)carbamoyl)-2-(1-(trifluoromethyl)cyclopropane-1-carbonyl)-2,6-diazaspiro[3.4]octane-6-carboxylate (120 mg, 23%) as a white solid. LCMS m/z=844.3 [M+H]⁺; ¹H NMR (400 MHz, DMSO-d₆) & 8.26 (s, 1H), 7.70 (d, J=8 Hz, 2H), 7.55 (d, J=8 Hz, 2H), 4.84-4.83 (m, 1H), 4.21 (s, 1H), 3.63-3.38 (m, 19H), 3.22-3.20 (m, 1H), 2.38-2.32 (m, 4H), 1.82-1.50 (m, 6H), 1.39 (s, 9H), 1.36-1.33 (m, 3H), 1.14 (s, 3H), 1.00 (t, J=7.6 Hz, 3H).

Step 4: (S)—N-((2S,3R)-3-((2-oxabicyclo[2.2.2]octan-4-yl)methoxy)-1-oxo-1-(4-(4-(trifluoromethyl)benzyl)piperazin-1-yl)butan-2-yl)-2-(1-(trifluoromethyl)cyclopropane-1-carbonyl)-2,6-diazaspiro[3.4]octane-8-carboxamide: To a solution of tert-butyl (S)-8-(((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamoyl)-2-(1-(trifluoromethyl)cyclopropane-1-carbonyl)-2,6-diazaspiro[3.4]octane-6-carboxylate in DCM (1 mL) was added TFA (0.5 mL) and the rection stirred at room temperature for 2 h. The solvent was removed under vacuum to afford S)—N-((2S,3R)-3-((2-oxabicyclo[2.2.2]octan-4-yl)methoxy)-1-oxo-1-(4-(4-(trifluoromethyl)benzyl)piperazin-1-yl)butan-2-yl)-2-(1-(trifluoromethyl)cyclopropane-1-carbonyl)-2,6-diazaspiro[3.4]octane-8-carboxamide as a white solid which was used without purification. LCMS m/z=744.15 [M+H]⁺.

Step 5: methyl 2-((4-((S)-8-(((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoate: To a solution of (S)—N-((2S,3R)-3-((2-oxabicyclo[2.2.2]octan-4-yl)methoxy)-1-oxo-1-(4-(4-(trifluoromethyl)benzyl)piperazin-1-yl)butan-2-yl)-2-(1-(trifluoromethyl)cyclopropane-1-carbonyl)-2,6-diazaspiro[3.4]octane-8-carboxamide (44 mg, 0.059 mmol) in DCM (1 mL) was added DIPEA (30 mg, 0.2366 mmol), 1-(2-(methoxycarbonyl)benzyl)-1H-pyrazole-4-carboxylic acid (15 mg, 0.059 mmol) and HATU (22 mg, 0.059 mmol) and the reaction stirred for 2 h. The mixture was concentrated under vacuum and the residue purified by RP-column (72% ACN in water) to afford methyl 2-((4-((S)-8-(((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoate (2.6 mg, 13%) as a yellow oil. LCMS m/z=986.4 [M+H]⁺; ¹H NMR (400 MHz, DMSO-d₆) & 8.37-8.3 (m, 3H), 7.92-7.89 (m, 1H), 7.68 (s, 1H), 7.53-7.42 (m, 4H), 6.82 (d, J=7.6 Hz, 1H), 5.72 (s, 211), 4.85 (s, 1H), 4.20-4.00 (m, 4H), 3.87 (s, 3H), 3.74-3.44 (m, 20H), 1.81 (s, 1H), 1.55-1.50 (m, 4H), 1.36-1.34 (m, 2H), 1.23-1.15 (m, 5H), 1.01 (t, J=7.2 Hz, 3H).

Step 6: 2-((4-((S)-8-(((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid: To a solution of methyl 2-((4-((S)-8-(((2S,3R)-3-((2-oxabicyclo[2.2.2]octan-4-yl)methoxy)-1-oxo-1-(4-(4-(trifluoromethyl) benzyl)piperazin-1-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoate (90 mg, 0.09 mmol) in a mixture of THE (4 mL), MeOH (1 mL) and water (1 mL) at 0° C. was added LiOH (4 mg, 0.23 mmol). The reaction mixture was stirred at room temperature for 2 h then was diluted with EtOAc and water. The aqueous was collected, the pH to 3-4 with 1M HCl and the aqueous extracted with EtOAc. The combined organic layers were concentrated and the residue obtained purified by prep-HPLC to afford 2-((4-((S)-8-(((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (22.4 mg, 25) as a white solid. LCMS m/z=972.3 [M+H]⁺; ¹H NMR (400 MHz, DMSO-d₆) & 8.35-8.3 (m, 2H), 7.93-7.84 (m, 2H), 7.68 (s, 2H), 7.543-7.38 (m, 4H), 6.76-6.74 (m, 1H), 5.75 (s, 2H), 4.85 (s, 1H), 4.20-3.89 (m, 25H), 1.81 (s, 1H), 1.56-1.50 (m, 4H), 1.36-1.15 (i, 7H), 1.01 (t, J=7.2 Hz, 3H).

Table I-8: The compound listed in Table I-8 were synthesized according to the procedures outlined for I-9 using the appropriate commercially available reagents and/or intermediates described elsewhere.

TABLE I-8

| Ex- | | | |
|---|---|---|---|
| ample | | | |
| Num- | | | |
| ber | Compound | ¹H NMR | LCMS |
| I-8 | 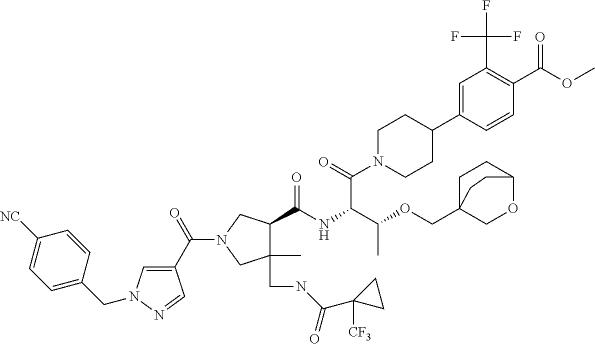 [SMILES: COC(=O)c1ccc(C2CCN(C(=O)[C@@H](NC(=O)[C@@H]3CN(C(=O)c4cnn(Cc5ccc(C#N)cc5)c4)CC3(C)CNC(=O)C3(C(F)(F)F)CC3)[C@@H](C)OCC34CCC(CC3)OC4)CC2)cc1C(F)(F)F] | ¹H NMR (400 MHz, DMSO-d₆) δ 8.37 (m, 2H), 7.89-7.60 (m, 6H), 7.39 (d, J = 7.4 Hz, 2H), 5.47 (s, 2H), 4.92 (s, 1H), 4.56 (s, 1H), 4.19 (m, 6H), 3.85 (s, 3H), 3.76 (s, 1H), 3.59 (m, 5H), 3.44 (m, 2H), 3.14 (d, J = 9.4 Hz, 2H), 3.01 (s, 2H), 2.67 (m, 1H), 1.86 (m, 3H), 1.54 (s, 5H), 1.43-1.35 (m, 2H), 1.27-1.16 (m, 6H), 1.03 (d, J = 9.8 Hz, 3H). | m/z = 996.4 [M + H]⁺ |

### Synthesis of methyl 2-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoate (I-11)

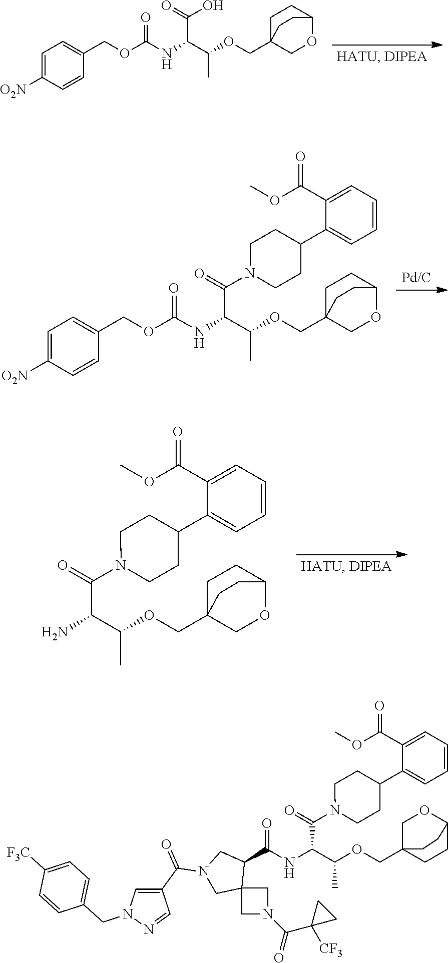 [SMILES: C[C@@H](OCC12CCC(CC1)OC2)[C@H](NC(=O)OCc1ccc([N+](=O)[O-])cc1)C(=O)O ; COC(=O)c1ccccc1C1CCN(C(=O)[C@@H](NC(=O)OCc2ccc([N+](=O)[O-])cc2)[C@@H](C)OCC23CCC(CC2)OC3)CC1 ; COC(=O)c1ccccc1C1CCN(C(=O)[C@@H](N)[C@@H](C)OCC23CCC(CC2)OC3)CC1 ; COC(=O)c1ccccc1C1CCN(C(=O)[C@@H](NC(=O)[C@@H]2CN(C(=O)c3cnn(Cc4ccc(C(F)(F)F)cc4)c3)CC23CN(C(=O)C2(C(F)(F)F)CC2)C3)[C@@H](C)OCC23CCC(CC2)OC3)CC1]

Step 1: methyl 2-(1-(O-((2-oxabicyclo[2.2.2]octan-4-yl)methyl)-N-(((4-nitrobenzyl)oxy) carbonyl)-L-threonyl)piperidin-4-yl)benzoate: To a solution of O-((2-oxabicyclo[2.2.2]octan-4-yl)methyl)-N-(((4-nitrobenzyl)oxy)carbonyl)-L-threonine (200 mg, 0.5 mmol) in DCM (4 mL) was added HATU (216 mg, 0.6 mmol) and the mixture stirred at room temperature for 5 mins. Methyl 2-(piperidin-4-yl)benzoate (117 mg, 0.6 mmol) and DIPEA (184 mg, 1.4 mmol) were added and stirring continued for 2 h. The mixture was diluted with water (30 mL) and extracted with DCM (50 mL×3). The combined organic layers were washed with brine, dried over Na₂SO₄, filtered and concentrated. The residue obtained was purified by prep-TLC (eluent: Pet. Ether:EtOAc=1:1) to afford methyl 2-(1-(O-((2-oxabicyclo[2.2.2]octan-4-yl)methyl)-N-(((4-nitrobenzyl)oxy)carbonyl)-L-threonyl)piperidin-4-yl)benzoate (139 mg, 47.2%) as a yellow oil. LCMS m/z=624.2 [M+H]⁺; ¹H NMR (400 MHz, DMSO-d6) δ 8.24 (d, J=8.0 Hz, 1H), 8.13 (d, J=8.2 Hz, 1H), 7.75-7.58 (m, 3H), 7.32 (s, 4H), 5.18 (s, 1H), 4.51 (s, 2H), 4.19 (s, 1H), 3.85 (d, J=3.4 Hz, 3H), 3.63-3.55 (m, 2H), 3.52-3.46 (m, 4H), 3.43-3.37 (m, 3H), 1.80 (s, 2H), 1.65 (d, J=11.4 Hz, 1H), 1.55-1.45 (m, 5H), 1.39-1.25 (m, 4H), 1.17 (s, 1H), 1.07 (s, 3H).

Step 2: methyl 2-(1-(O-((2-oxabicyclo[2.2.2]octan-4-yl)methyl)-L-threonyl)piperidin-4-yl)benzoate: To a solution of methyl 2-(1-(O-((2-oxabicyclo[2.2.2]octan-4-yl)methyl)-N-(((4-nitrobenzyl)oxy)carbonyl)-L-threonyl)piperidin-4-yl)benzoate (139 mg, 0.2 mmol) in MeOH (4 mL) was added Pd/C (10%, 13.9 mg) and the reaction heated at 50° C. under a H₂ atmosphere for 2 h. The catalyst was removed by filtration through celite and the filtrate concentrated to afford methyl 2-(1-(O-((2-oxabicyclo[2.2.2]octan-4-yl)methyl)-L-threonyl)piperidin-4-yl)benzoate (125 mg) as a yellow oil. LCMS m/z=445.2 [M+H]⁺; ¹H NMR (400 MHz, DMSO-d6) δ 7.70 (d, J=7.6 Hz, 1H), 7.53 (s, 1H), 7.37 (d, J=22.0 Hz, 1H), 6.81 (d, J=8.0 Hz, 1H), 6.46 (d, J=8.4 Hz, 1H), 4.75 (s, 1H), 4.19 (d, J=26.4 Hz, 1H), 3.85 (s, 3H), 3.76-3.61 (m, 2H), 3.54 (t, J=10.2 Hz, 2H), 3.46 (s, 4H), 2.11 (s, 2H), 1.86-1.73 (m, 3H), 1.55 (s, 5H), 1.47-1.24 (m, 6H), 1.05 (d, J=16.0 Hz, 2H).

Step 3: methyl 2-(1-(O-((2-oxabicyclo[2.2.2]octan-4-yl)methyl)-N—((S)-6-(1-(4-(trifluoromethyl) benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoate: To a solution of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (118 mg, 0.2 mmol) in DCM (3 mL) was added HATU (99 mg, 0.6 mmol) and the mixture stirred at room temperature for 5 mins. Methyl 2-(1-(O-((2-oxabicyclo[2.2.2]octan-4-yl)methyl)-L-threonyl)piperidin-4-yl)benzoate (115 mg, 0.3 mmol) and DIPEA (84 mg, 0.7 mmol) were added and stirring continued for 2 h. The mixture was diluted with water (30 mL) and extracted with DCM (40 mL×3). The combined organic layers were washed with brine, dried over Na₂SO₄, filtered and concentrated. The residue obtained was purified by prep-HPLC to afford methyl 2-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoate (70.3 mg, 33%) as a white solid. LCMS m/z=971.4 [M+H]⁺; ¹H NMR (400 MHz, DMSO-d6) δ 8.40 (s, 2H), 7.87 (s, 1H), 7.71 (s, 3H), 7.39 (d, J=29.4 Hz, 5H), 5.47 (s, 2H), 4.92 (s, 1H), 4.56 (s, 1H), 4.11 (d, J=80.0 Hz, 5H), 3.84 (s, 5H), 3.47 (d, J=11.2 Hz, 8H), 3.20-3.00 (m, 2H), 2.63 (s, 1H), 1.81 (s, 4H), 1.62-1.36 (m, 8H), 1.20 (d, J=30.4 Hz, 5H), 1.05 (d, J=17.6 Hz, 3H).

Table I-9: The compound listed in Table I-9 were synthesized according to the procedures outlined for I-11 using the appropriate commercially available reagents and/or intermediates described elsewhere.

TABLE I-9

| Ex- | | | |
|---|---|---|---|
| am- | | | |
| ple | | | |
| Num- | | | |
| ber | Compound | ¹H NMR | LCMS |
| I-2 | 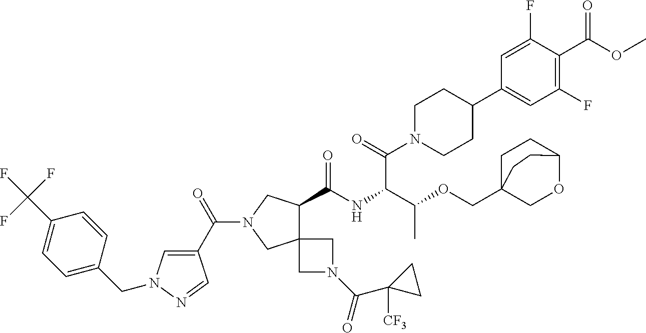 [SMILES: COC(=O)c1c(F)cc(C2CCN(C(=O)[C@@H](NC(=O)[C@@H]3CN(C(=O)c4cnn(Cc5ccc(C(F)(F)F)cc5)c4)CC34CN(C(=O)C3(C(F)(F)F)CC3)C4)[C@@H](C)OCC34CCC(CC3)OC4)CC2)cc1F] | ¹H NMR (400 MHz, DMSO-d₆) δ 8.36 (d, J = 25.2 Hz, 2H), 7.85 (d, J = 15.2 Hz, 1H), 7.71 (d, J = 8.0 Hz, 2H), 7.43 (d, J = 7.8 Hz, 2H), 7.15 (d, J = 10.6 Hz, 2H), 5.47 (s, 2H), 4.90 (s, 1H), 4.53 (s, 1H), 3.86 (s, 11H), 3.59 (d, J = 30.2 Hz, 6H), 3.19-2.84 (m, 4H), 2.65 (d, J = 13.0 Hz, 1H), 1.82 (s, 4H), 1.46 (d, J = 58.6 Hz, 8H), 1.19 (d, J = 29.4 Hz, 4H), 1.03 (d, J = 15.7 Hz, 3H). | m/z = 1007.3 [M + H]⁺ |
| I-5 | 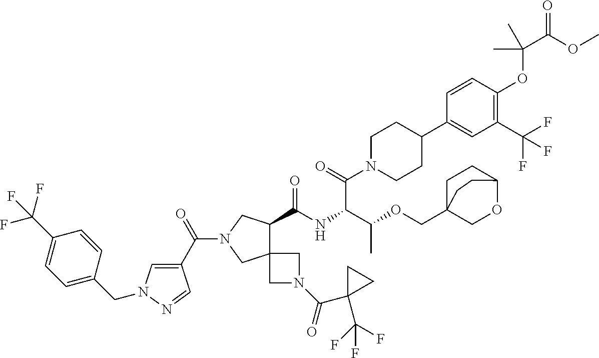 [SMILES: COC(=O)C(C)(C)Oc1ccc(C2CCN(C(=O)[C@@H](NC(=O)[C@@H]3CN(C(=O)c4cnn(Cc5ccc(C(F)(F)F)cc5)c4)CC34CN(C(=O)C3(C(F)(F)F)CC3)C4)[C@@H](C)OCC34CCC(CC3)OC4)CC2)cc1C(F)(F)F] | ¹H NMR (400 MHz, Chloroform-d) δ 7.89 (s, 1H), 7.84 (s, 1H), 7.62 (d, J = 8.0 Hz, 2H), 7.39-7.31 (m, 3H), 7.19 (d, J = 8.6 Hz, 1H), 6.91 (d, J = 7.6 Hz, 1H), 6.76- 6.69 (m, 1H), 5.37 (s, 2H), 4.97 (s, 1H), 4.80-4.67 (m, 1H), 4.40-3.83 (m, 9H), 3.79 (s, 4H), 3.69- 2.93 (m, 7H), 2.83- 2.60 (m, 2H), 2.04- 1.70 (m, 6H), 1.61 (s, 9H), 1.53-1.26 (m, 4H), 1.22 (d, J = 2.8 Hz, 3H), 1.17-1.02 (m, 3H). | m/z = 1097.3 [M + H]⁺ |
| I-13 | 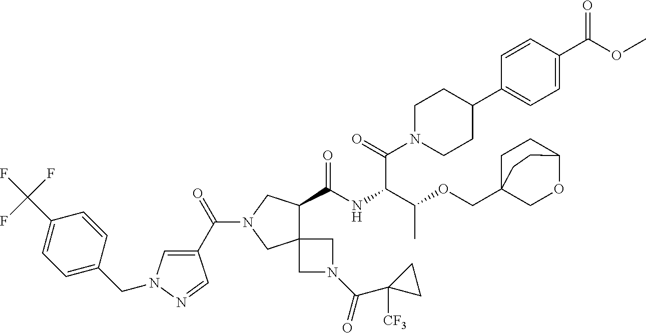 [SMILES: COC(=O)c1ccc(C2CCN(C(=O)[C@@H](NC(=O)[C@@H]3CN(C(=O)c4cnn(Cc5ccc(C(F)(F)F)cc5)c4)CC34CN(C(=O)C3(C(F)(F)F)CC3)C4)[C@@H](C)OCC34CCC(CC3)OC4)CC2)cc1] | ¹H NMR (400 MHz, DMSO-d₆) δ 8.49- 8.28 (m, 2H), 7.96- 7.80 (m, 3H), 7.75- 7.67 (m, 2H), 7.48- 7.33 (m, 4H), 5.47 (s, 2H), 4.92 (s, 1H), 4.54 (s, 1H), 4.27- 4.07 (m, 3H), 4.01 (s, 2H), 3.83 (s, 7H), 3.66-3.52 (m, 5H), 3.20-3.09 (m, 1H), 3.05-2.84 (m, 1H), 1.90-1.76 (m, 3H), 1.63-1.49 (m, 5H), 1.44-1.33 (m, 3H), 1.31-1.14 (m, 5H), 1.09-1.00 (m, 3H). | m/z = 971.4 [M + H]⁺ |

### Synthesis of 2-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl) cyclopropane-1-carbonyl)-2,6-diazaspiro[3.4]octane-8-carbonyl)-L-threonyl)piperidin-4-yl)benzoic acid (I-3)

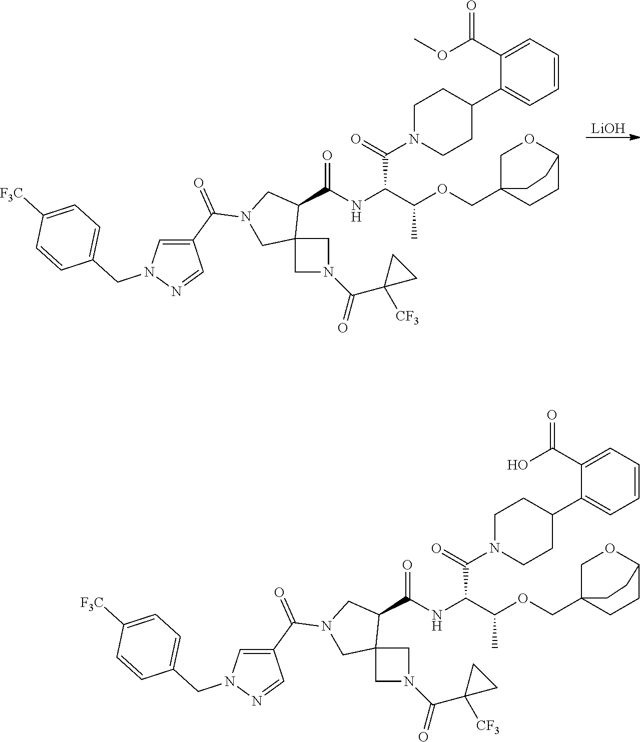

To a solution of methyl 2-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoate (50 mg, 0.1 mmol) in a mixture of THF (2 mL), MeOH (0.5 mL) and water (0.5 mL) was added lithium hydroxide monohydrate (5 mg, 0.3 mmol). The reaction was stirred at room temperature for 1 h then heated at 30° C. for 1 h. The reaction was diluted with water (15 mL) and extracted with EtOAc (20 mL). The aqueous layer was collected and acidified with 1M HCl to pH ~2 then extracted with EtOAc (25 mL×2). The combined organic layers were washed with brine, dried over Na₂SO₄, filtered and concentrated. The residue obtained was purified by prep-HPLC to afford 2-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoic acid (11 mg, 22%) as a white solid. LCMS m/z=957.3 [M+H]⁺; 1H NMR (400 MHz, DMSO-d6) δ 8.41 (s, 1H), 7.85 (d, J=14.2 Hz, 1H), 7.70 (s, 3H), 7.43 (d, J=8.0 Hz, 3H), 7.31 (s, 2H), 5.47 (s, 2H), 4.91 (s, 1H), 4.56 (s, 1H), 4.24-3.75 (m, 9H), 3.60 (d, J=23.4 Hz, 8H), 3.26 (s, 2H), 1.82 (s, 3H), 1.62-1.35 (m, 9H), 1.20 (d, J=31.0 Hz, 5H), 1.06 (s, 3H).

Table I-10: The compound listed in Table I-10 were synthesized according to the procedures outlined for I-3 using the appropriate commercially available reagents and/or intermediates described elsewhere.

TABLE I-10

| Ex- | | | |
|---|---|---|---|
| ample | | | |
| Num- | | | |
| ber | Compound | ¹H NMR | LCMS |
| I-4 | 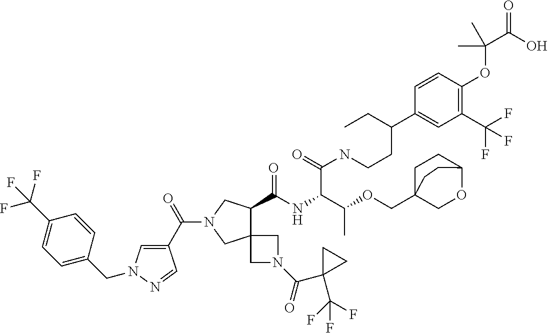 [SMILES: CCC(CCNC(=O)[C@@H](NC(=O)[C@@H]1CN(C(=O)c2cnn(Cc3ccc(C(F)(F)F)cc3)c2)CC12CN(C(=O)C1(C(F)(F)F)CC1)C2)[C@@H](C)OCC12CCC(CC1)OC2)c1ccc(OC(C)(C)C(=O)O)c(C(F)(F)F)c1] | ¹H NMR (400 MHz, DMSO-d₆) δ 8.48- 8.28 (m, 2H), 7.85 (d, J = 14.6 Hz, 1H), 7.71 (d, J = 7.8 Hz, 2H), 7.49-7.35 (m, 4H), 6.89 (s, 1H), 5.48 (s, 2H), 5.03- 4.75 (m, 1H), 4.61- 4.44 (m, 1H), 4.40- 3.48 (m, 15H), 3.21- 2.93 (m, 3H), 2.82 (s, 1H), 1.82 (s, 4H), 1.57-1.46 (m, 11H), 1.41-1.31 (m, 3H), 1.16 (s, 3H), 1.10- 0.97 (m, 4H). | m/z = 1083.1 [M + H]⁺ |
| I-12 | 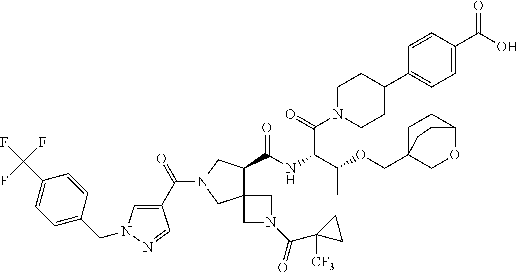 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)N1CCC(c2ccc(C(=O)O)cc2)CC1] | ¹H NMR (400 MHz, DMSO-d₆) δ 8.59- 8.23 (m, 2H), 7.93- 7.78 (m, 3H), 7.75- 7.65 (m, 2H), 7.43 (d, J = 7.8 Hz, 2H), 7.30 (s, 2H), 5.47 (s, 2H), 4.90 (s, 1H), 4.54 (s, 1H), 4.45-4.07 (m, 4H), 4.05-3.85 (m, 4H), 3.82-3.70 (m, 3H), 3.66-3.59 (m, 5H), 3.58-3.55 (m, 3H), 2.94-2.80 (m, 2H), 1.82 (s, 3H), 1.64-1.48 (m, 5H), 1.45-1.33 (m, 3H), | m/z = 957.4 [M + H]⁺ |
| | | 1.31-1.12 (m, 5H), | |
| | | 1.11-0.98 (m, 3H). | |
| I-104 | 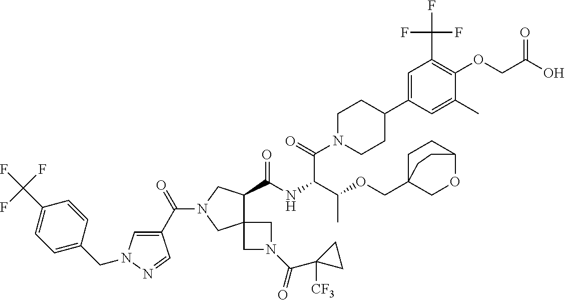 [SMILES: Cc1cc(C2CCN(C(=O)[C@@H](NC(=O)[C@@H]3CN(C(=O)c4cnn(Cc5ccc(C(F)(F)F)cc5)c4)CC34CN(C(=O)C3(C(F)(F)F)CC3)C4)[C@@H](C)OCC34CCC(CC3)OC4)CC2)cc(C(F)(F)F)c1OCC(=O)O] | ¹H NMR (400 MHz, Methanol-d₄) δ 8.32- 8.17 (m, 1H), 7.99- 7.91 (m, 1H), 7.69- 7.60 (m, 2H), 7.47- 7.28 (m, 4H), 5.49 (s, 2H), 5.02-4.93 (m, 1H), 4.65-4.55 (m, 2H), 4.35 (s, 2H), 4.25-4.16 (m, 2H), 4.10-3.98 (m, 2H), 3.93-3.56 (m, 6H), 3.50-3.34 (m, 3H), 3.27-3.18(m, 2H), 3.09-2.98(m, 1H), 2.93-2.85 (m, 1H), 2.80-2.72 (m, 1H), 2.37-2.31 (m, 3H), 2.04-1.84 (m, 4H), | m/z = 1069.2 [M + H]⁺ |
| | | 1.76-1.60 (m, 5H), | |
| | | 1.56-1.44 (m, 3H), | |
| | | 1.31-1.28 (m, 1H), | |
| | | 1.27-1.22 (m, 3H), | |
| | | 1.21-1.14 (m, 3H). | |

### Synthesis of 2-((4-((S)-8-(((2S,3R)-3-((2-oxabicyclo[2.2.2]octan-4-yl)methoxy)-1-(9-((allyloxy)carbonyl)-3,9-diazaspiro[5.5]undecan-3-yl)-1-oxobutan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (I-7)

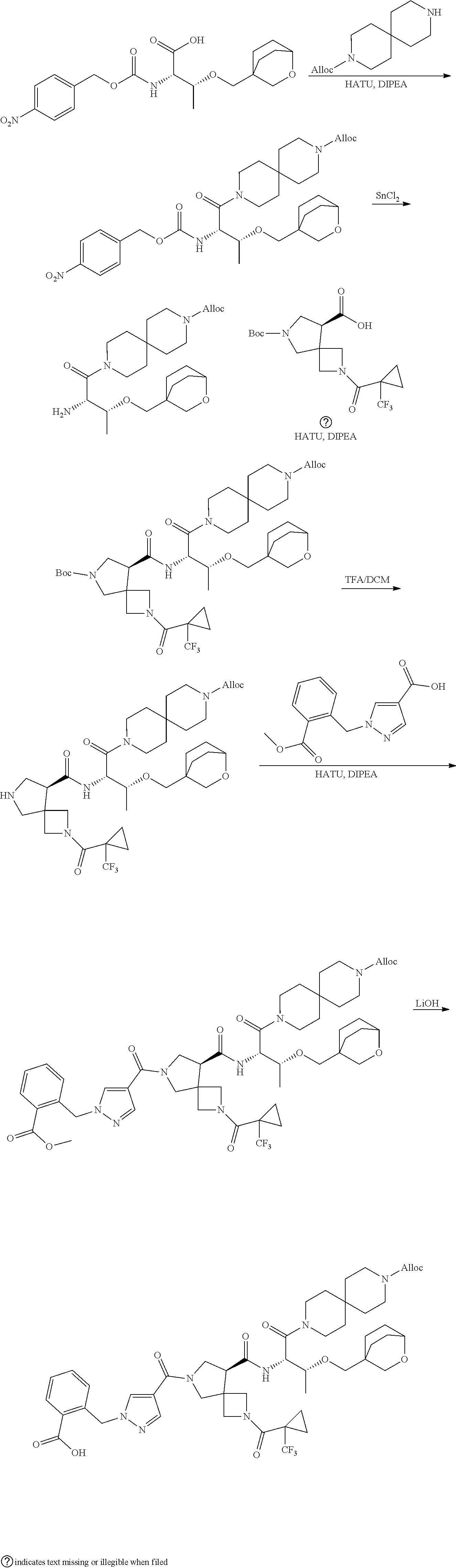

Step 1: allyl 9-(O-((2-oxabicyclo[2.2.2]octan-4-yl)methyl)-N-(((4-nitrobenzyl)oxy)carbonyl)-L-threonyl)-3,9-diazaspiro[5.5]undecane-3-carboxylate: To a solution of O-((2-oxabicyclo[2.2.2]octan-4-yl)methyl)-N-(((4-nitrobenzyl)oxy)carbonyl)-L-threonine (624 mg, 1.48 mmol) in DCM (8 mL) was added HATU (618 mg, 1.63 mmol) and the mixture was stirred at room temperature for 30 min. Allyl 3,9-diazaspiro[5.5]undecane-3-carboxylate (350 mg, 1.48 mmol) and DIPEA (764 mg, 5.91 mmol) were added and stirring continued for 2 h. The mixture was diluted with water (20 mL) and extracted with DCM (50 mL×3). The combined organic layers were washed with brine, dried over Na₂SO₄, filtered and concentrated. The residue obtained was purified by silica gel column (eluent: DCM:MeOH=80:1) to afford allyl 9-(O-((2-oxabicyclo[2.2.2]octan-4-yl)methyl)-N-(((4-nitrobenzyl)oxy)carbonyl)-L-threonyl)-3,9-diazaspiro[5.5]undecane-3-carboxylate (650 mg, 69%) as a yellow oil. LCMS m/z=643.2 [M+H]⁺; ¹H NMR (400 MHz, DMSO-d₆) δ 8.23 (d, J=8.8 Hz, 2H), 7.62 (d, J=8.4 Hz, 2H), 7.44 (d, J=8.8 Hz, 1H), 5.92 (m, 1H), 5.26 (m, 1H), 5.20-5.15 (m, 3H), 4.51 (d, J=5.2 Hz, 2H), 4.44 (dd, J=9.0, 6.8 Hz, 1H), 3.49 (m, 5H), 3.37 (m, 6H), 3.13 (d, J=9.2 Hz, 1H), 2.98-2.85 (m, 2H), 1.79 (m, 2H), 1.53-1.47 (m, 4H), 1.45-1.42 (m, 2H), 1.34-1.29 (m, 3H), 1.28-1.23 (m, 3H), 1.02 (t, J=6.6 Hz, 3H), 0.87 (t, J=7.4 Hz, 2H).

Step 2: allyl 9-(O-((2-oxabicyclo[2.2.2]octan-4-yl)methyl)-L-threonyl)-3,9-diazaspiro[5.5]undecane-3-carboxylate: To a solution of allyl 9-(O-((2-oxabicyclo[2.2.2]octan-4-yl)methyl)-N-(((4-nitrobenzyl)oxy)carbonyl)-L-threonyl)-3,9-diazaspiro[5.5]undecane-3-carboxylate (650 mg, 1.01 mmol) in EtOH (8 mL) was added SnCl₂ (959 mg, 5.06 mmol), the reaction heated at 80° C. for 2 h. The solvent was removed under reduced pressure and the residue obtained purified by column (eluent: DCM:MeOH=10:1) to afford allyl 9-(O-((2-oxabicyclo[2.2.2]octan-4-yl)methyl)-L-threonyl)-3,9-diazaspiro[5.5]undecane-3-carboxylate (350 mg, 75%) as a yellow oil. LCMS m/z=464.2 [M+H]⁺; ¹H NMR (400 MHz, DMSO-d₆) δ 5.92 (m, 1H), 5.26 (dd, J=17.2, 1.8 Hz, 1H), 5.18 (dd, J=10.4, 1.8 Hz, 1H), 4.52-4.49 (m, 2H), 3.64 (m, 1H), 3.56-3.52 (m, 2H), 3.47 (d, J=5.6 Hz, 4H), 3.37 (m, 8H), 1.88-1.76 (m, 2H), 1.58-1.50 (m, 4H), 1.45-1.39 (m, 7H), 1.06 (dd, J=6.2, 4.6 Hz, 2H), 1.00 (d, J=6.2 Hz, 1H), 0.87 (t, J=7.4 Hz, 3H).

Step 3: allyl 9-(O-((2-oxabicyclo[2.2.2]octan-4-yl)methyl)-N—((S)-6-(tert-butoxycarbonyl)-2-(1-(trifluoromethyl)cyclopropane-1-carbonyl)-2,6-diazaspiro[3.4]octane-8-carbonyl)-L-threonyl)-3,9-diazaspiro[5.5]undecane-3-carboxylate: To a solution of (S)-6-(tert-butoxycarbonyl)-2-(1-(trifluoromethyl)cyclopropane-1-carbonyl)-2,6-diazaspiro[3.4]octane-8-carboxylic acid (203 mg, 0.52 mmol) in DCM (8 mL) was added HATU (270 mg, 0.71 mmol) and the mixture was stirred at room temperature for 30 min. Allyl 9-(O-((2-oxabicyclo[2.2.2]octan-4-yl)methyl)-L-threonyl)-3,9-diazaspiro[5.5]undecane-3-carboxylate (300 mg, 0.65 mmol) and DIPEA (250 mg, 1.94 mmol) were added and stirring continued for 2 h. The mixture was diluted with water (20 mL) and extracted with DCM (50 mL×3). The combined organic layers were washed with brine, dried over Na₂SO₄, filtered and concentrated. The residue obtained was purified by silica gel column (eluent: DCM:MeOH=50:1) to afford allyl 9-(O-((2-oxabicyclo[2.2.2]octan-4-yl)methyl)-N—((S)-6-(tert-butoxycarbonyl)-2-(1-(trifluoromethyl)cyclopropane-1-carbonyl)-2,6-diazaspiro[3.4]octane-8-carbonyl)-L-threonyl)-3,9-diazaspiro[5.5]undecane-3-carboxylate (220 mg, 40%) as a yellow oil. LCMS m/z=738.2 [M+H-100]⁺; ¹H NMR (400 MHz, DMSO-d₆) δ 8.23 (t, J=9.4 Hz, 1H), 5.92 (m, 1H), 5.26 (dd, J=17.2, 1.8 Hz, 1H), 5.18 (dd, J=10.4, 1.6 Hz, 1H), 4.84 (dd, J=8.6, 4.4 Hz, 1H), 4.54-4.48 (m, 2H), 3.65-3.60 (m, 1H), 3.53 (d, J=5.8 Hz, 3H), 3.47 (q, J=5.4 Hz, 7H), 3.42-3.30 (m, 12H), 1.83 (d, J=10.4 Hz, 1H), 1.58-1.44 (m, 6H), 1.40 (d, J=11.4 Hz, 14H), 1.34-1.27 (m, 3H), 1.24 (s, 2H), 1.15 (s, 1H), 1.00 (t, J=6.8 Hz, 2H), 0.87 (t, J=7.2 Hz, 3H).

Step 4: allyl 9-(O-((2-oxabicyclo[2.2.2]octan-4-yl)methyl)-N—((S)-2-(1-(trifluoromethyl) cyclopropane-1-carbonyl)-2,6-diazaspiro[3.4]octane-8-carbonyl)-L-threonyl)-3,9-diazaspiro[5.5]undecane-3-carboxylate: To a solution of allyl 9-(O-((2-oxabicyclo[2.2.2]octan-4-yl)methyl)-N—((S)-6-(tert-butoxycarbonyl)-2-(1-(trifluoromethyl)cyclopropane-1-carbonyl)-2,6-diazaspiro[3.4]octane-8-carbonyl)-L-threonyl)-3,9-diazaspiro[5.5]undecane-3-carboxylate (120 mg, 0.14 mmol) in DCM (3 mL) was added TFA (1 mL) and the reaction stirred at room temperature for 2 h. The solvent was removed under vacuum to afford allyl 9-(O-((2-oxabicyclo[2.2.2]octan-4-yl)methyl)-N—((S)-2-(1-(trifluoromethyl)cyclopropane-1-carbonyl)-2,6-diazaspiro[3.4]octane-8-carbonyl)-L-threonyl)-3,9-diazaspiro[5.5]undecane-3-carboxylate (106 mg, 100%) which was used directly in the next step. LCMS m/z=738.2 [M+H]⁺.

Step 5: allyl 9-(O-((2-oxabicyclo[2.2.2]octan-4-yl)methyl)-N—((S)-6-(1-(2-(methoxycarbonyl) benzyl)-1H-pyrazole-4-carbonyl)-2-(1-(trifluoromethyl)cyclopropane-1-carbonyl)-2,6-diazaspiro[3.4]octane-8-carbonyl)-L-threonyl)-3,9-diazaspiro[5.5]undecane-3-carboxylate: To a solution of 1-(2-(methoxycarbonyl)benzyl)-1H-pyrazole-4-carboxylic acid (37 mg, 0.14 mmol) in DCM (3 mL) was added HATU (60 mg, 0.16 mmol) and the mixture was stirred at room temperature for 30 min. Allyl 9-(O-((2-oxabicyclo[2.2.2]octan-4-yl)methyl)-N—((S)-2-(1-(trifluoromethyl)cyclopropane-1-carbonyl)-2,6-diazaspiro[3.4]octane-8-carbonyl)-L-threonyl)-3,9-diazaspiro[5.5]undecane-3-carboxylate (106 mg, 0.14 mmol) and DIPEA (74 mg, 0.57 mmol) were added and stirring continued for 2 h. The mixture was diluted with water (20 mL) and extracted with DCM (50 mL×3). The combined organic layers were washed with brine, dried over Na₂SO₄, filtered and concentrated. The residue obtained was purified by prep-TLC (eluent: DCM:MeOH=15:1) to afford allyl 9-(O-((2-oxabicyclo[2.2.2]octan-4-yl)methyl)-N—((S)-6-(1-(2-(methoxycarbonyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)-3,9-diazaspiro[5.5]undecane-3-carboxylate (98 mg, 40%) as a yellow solid. LCMS m/z=980.5 [M+H]⁺; ¹H NMR (400 MHz, DMSO-d₆) δ 8.31 (d, J=19.0 Hz, 2H), 7.94-7.84 (m, 2H), 7.53 (d, J=8.2 Hz, 1H), 7.44 (t, J=7.6 Hz, 1H), 6.81 (d, J=7.2 Hz, 1H), 5.92 (m, 1H), 5.72 (d, J=5.8 Hz, 2H), 5.22 (dd, J=34.6, 13.8 Hz, 2H), 4.85 (m, 1H), 4.52-4.49 (m, 2H), 4.20 (m, 2H), 4.00 (m, 2H), 3.87 (d, J=3.2 Hz, 3H), 3.73 (m, 2H), 3.62 (m, 2H), 3.54 (s, 2H), 3.49-3.42 (m, 5H), 3.40-3.34 (m, 6H), 3.12 (d, J=9.6 Hz, 1H), 2.98 (d, J=9.2 Hz, 1H), 2.04-1.95 (m, 1H), 1.82 (m, 2H), 1.56-1.48 (m, 4H), 1.41-1.34 (m, 9H), 1.31 (d, J=7.8 Hz, 2H), 1.01 (t, J=6.8 Hz, 3H), 0.87 (t, J=7.4 Hz, 2H).

Step 6: 2-((4-((S)-8-(((2S,3R)-3-((2-oxabicyclo[2.2.2]octan-4-yl)methoxy)-1-(9-((allyloxy) carbonyl)-3,9-diazaspiro[5.5]undecan-3-yl)-1-oxobutan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid: To a solution of allyl 9-(O-((2-oxabicyclo[2.2.2]octan-4-yl)methyl)-N—((S)-6-(1-(2-(methoxycarbonyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)-3,9-diazaspiro[5.5]undecane-3-carboxylate (90 mg, 0.09 mmol) in a mixture of THF, water and MeOH (2 mL/0.5 mL/0.5 mL) was added LiOH (12 mg, 0.28 mmol). The reaction mixture was stirred at room temperature for 3 h, then was diluted with water (20 mL). The aqueous layer was acidified to pH ~2 with 1M HCl and extracted with EtOAc (50 mL×3). The combined organic layers were washed with brine, dried over Na₂SO₄, filtered and concentrated to afford 2-((4-((S)-8-(((2S,3R)-3-((2-oxabicyclo[2.2.2]octan-4-yl)methoxy)-1-(9-((allyloxy)carbonyl)-3,9-diazaspiro[5.5]undecan-3-yl)-1-oxobutan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (77 mg, 43%) as a white solid. LCMS m/z=966.4 [M+H]⁺; ¹H NMR (400 MHz, Chloroform-d) δ 8.02 (m, 2H), 7.81 (d, J=25.4 Hz, 1H), 7.60-7.36 (m, 3H), 5.98-5.90 (m, 1H), 5.71 (m, 2H), 5.33-5.19 (m, 2H), 5.01 (m, 1H), 4.59 (dd, J=4.4, 2.8 Hz, 3H), 4.13 (m, 2H), 4.03-3.89 (m, 3H), 3.76 (d, J=11.6 Hz, 2H), 3.51 (m, 12H), 3.14 (d, J=36.4 Hz, 2H), 2.97 (dd, J=18.8, 9.2 Hz, 1H), 1.99 (m, 2H), 1.76 (m, 2H), 1.57 (m, 9H), 1.42 (d, J=6.8 Hz, 3H), 1.26 (d, J=2.6 Hz, 2H), 1.20 (m, 2H), 1.11 (d, J=6.8 Hz, 3H).

Table I-11: The compounds listed in Table I-11 were synthesized according to the procedures outlined for 2-((4-((S)-8-(((2S,3R)-3-((2-oxabicyclo[2.2.2]octan-4-yl)methoxy)-1-(9-((allyloxy)carbonyl)-3,9-diazaspiro[5.5]undecan-3-yl)-1-oxobutan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (I-7) using the appropriate commercially available reagents and/or intermediates described herein.

TABLE I-11

| Cmpd | | | |
|---|---|---|---|
| # | Structure | NMR Data | m/z |
| I-61 | 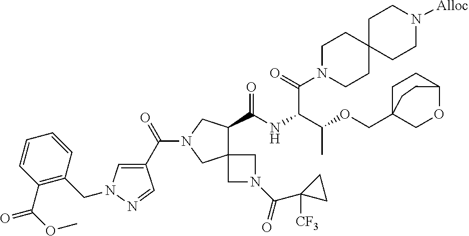 | ¹H NMR (400 MHz, DMSO-d6) δ 8.22 (d, J = 30.0 Hz, 2H), 7.91 (d, J = 7.6 Hz, 1H), 7.84 (s, 1H), 7.57- 7.51 (m, 1H), 7.44 (t, J = 7.6 Hz, 1H), 6.90 (d, J = 7.8 Hz, 1H), 5.97- 5.87 (m, 1H), 5.72 (d, J = 1.8 Hz, 2H), 5.30-5.16 (m, 2H), 4.84- 4.82 (m, 1H), 4.53-4.50 (m, 2H), 4.21-4.07 (m, 2H), 4.04-3.95 (m, 2H), 3.87 (s, 3H), 3.81-3.67 (m, 2H), 3.65-3.6 (m, 2H), 3.57-3.53 (m, 2H), 3.5-3.41 (m, 5H), 3.4- 3.3 (m, 6H), 3.12 (d, J = 9.4 Hz, 1H), 3.00 (d, J = 9.4 Hz, 1H), 1.89- 1.78 (m, 2H), 1.54 (t, J = 11.2 Hz, | m/z = 980.5 [M + H]⁺ |
| | | 4H), 1.44-1.36 (m, 9H), 1.23 (d, | |
| | | J = 13.2 Hz, 3H), 1.17 (d, J = 3.6 | |
| | | Hz, 2H), 1.03 (t, J = 6.8 Hz, 3H). | |

### Synthesis of 4-(1-(O-(cyclohexylmethyl)-N—((S)-6-(1-(cyclohexylmethyl)-1H-pyrazole-4-carbonyl)-2-((S)-2,2-dimethylcyclopropane-1-carbonyl)-2,6-diazaspiro[3.4]octane-8-carbonyl)-L-threonyl)piperidin-4-yl)benzoic acid (I-30)

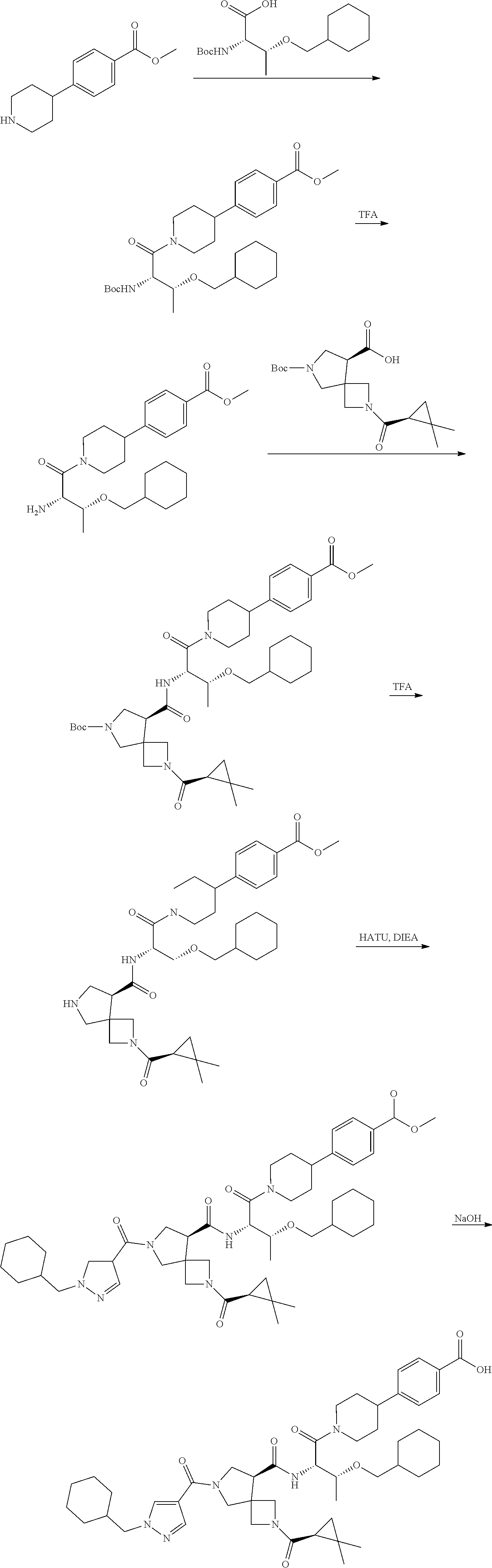

Step 1: methyl 4-(1-(N-(tert-butoxycarbonyl)-O-(cyclohexylmethyl)-L-threonyl)piperidin-4-yl)benzoate: To a solution of N-(tert-butoxycarbonyl)-O-(cyclohexylmethyl)-L-threonine (500 mg, 1.6 mmol) in DCM (5 mL) was added HATU (665 mg, 1.7 mmol) and DIEA (617 mg, 4.8 mmol) and the mixture stirred 30 min. Methyl 4-(piperidin-4-yl)benzoate (348 mg, 1.6 mmol) was added and stirring continued overnight. The mixture was diluted with water (30 mL) and extracted with EtOAc (30 mL×3). The combined organic layers were washed with brine, dried over Na₂SO₄, filtered and concentrated. The residue obtained was purified by column chromatography on silica gel (eluent: DCM:MeOH=40:1) to afford methyl 4-(1-(N-(tert-butoxycarbonyl)-O-(cyclohexylmethyl)-L-threonyl)piperidin-4-yl)benzoate (659 mg, 80%) as yellow oil. LCMS m/z=517.0 [M+H]⁺; ¹H NMR (400 MHz, CDCl₃) δ 7.98 (d, J=8.0 Hz, 2H), 7.25 (s, 2H), 5.62 (d, J=8.8 Hz, 1H), 4.85-4.71 (m, 2H), 4.25 (d, J=13.8 Hz, 1H), 3.91 (s, 3H), 3.63 (s, 1H), 3.39-3.07 (m, 3H), 2.82 (s, 2H), 1.91 (s, 2H), 1.71 (t, J=15.8 Hz, 7H), 1.44 (s, 10H), 1.28-1.09 (m, 6H), 0.96-0.82 (m, 2H).

Step 2: methyl 4-(1-(O-(cyclohexylmethyl)-L-threonyl)piperidin-4-yl)benzoate: To a solution of methyl 4-(1-(N-(tert-butoxycarbonyl)-O-(cyclohexylmethyl)-L-threonyl)piperidin-4-yl)benzoate (359 mg, 0.7 mmol) in DCM (2 mL) was added TFA (1 mL) and the mixture stirred at room temperature for 3 h. The solvent was removed under vacuum to afford methyl 4-(1-(0-(cyclohexylmethyl)-L-threonyl)piperidin-4-yl)benzoate (485 mg, quant.) as colorless oil which was used directly in the next step. LCMS m/z=417.1 [M+H]⁺.

Step 3: tert-butyl (S)-8-(((2S,3R)-3-(cyclohexylmethoxy)-1-(4-(4-(methoxycarbonyl) phenyl)piperidin-1-yl)-1-oxobutan-2-yl)carbamoyl)-2-((S)-2,2-dimethylcyclopropane-1-carbonyl)-2,6-diazaspiro[3.4]octane-6-carboxylate: To a solution of (S)-6-(tert-butoxycarbonyl)-2-((S)-2,2-dimethylcyclopropane-1-carbonyl)-2,6-diazaspiro[3.4]octane-8-carboxylic acid (422 mg, 1.2 mmol) in DCM (5 mL) was added HATU (502 mg, 1.3 mmol) and DIEA (465 mg, 3.6 mmol) and the reaction stirred for 30 min. Methyl 4-(1-(0-(cyclohexylmethyl)-L-threonyl)piperidin-4-yl)benzoate (500 mg, 1.2 mmol) was added and stirring continued overnight. The mixture was diluted with water (30 mL) and extracted with EtOAc (30 mL×3). The combined organic layers were washed with brine, dried over Na₂SO₄, filtered and concentrated. The residue obtained was purified by RP-column to afford tert-butyl (S)-8-(((2S,3R)-3-(cyclohexylmethoxy)-1-(4-(4-(methoxycarbonyl)phenyl)piperidin-1-yl)-1-oxobutan-2-yl)carbamoyl)-2-((S)-2,2-dimethylcyclopropane-1-carbonyl)-2,6-diazaspiro[3.4]octane-6-carboxylate (615 mg, 68%) as a white solid. LCMS m/z=751.7 [M+H]⁺; ¹H NMR (400 MHz, Chloroform-d) δ 7.99 (d, J=6.8 Hz, 2H), 7.26 (s, 2H), 6.86 (s, 1H), 5.04 (d, J=6.7 Hz, 1H), 4.78 (d, J=11.4 Hz, 1H), 4.23-3.51 (m, 13H), 3.37-3.11 (m, 3H), 3.00-2.83 (m, 2H), 2.76-2.66 (m, 1H), 2.01-1.89 (m, 2H), 1.76-1.63 (m, 7H), 1.46 (s, 9H), 1.21-1.07 (m, 14H), 0.94-0.82 (m, 3H), 0.77-0.70 (m, 1H).

Step 4: methyl 4-(1-(O-(cyclohexylmethyl)-N—((S)-2-((S)-2,2-dimethylcyclopropane-1-carbonyl)-2,6-diazaspiro[3.4]octane-8-carbonyl)-L-threonyl)piperidin-4-yl)benzoate: To a solution of tert-butyl (S)-8-(((2S,3R)-3-(cyclohexylmethoxy)-1-(4-(4-(methoxycarbonyl)phenyl)piperidin-1-yl)-1-oxobutan-2-yl)carbamoyl)-2-((S)-2,2-dimethylcyclopropane-1-carbonyl)-2,6-diazaspiro[3.4]octane-6-carboxylate (615 mg, 0.8 mmol) in DCM (6 mL) was added TFA (3 mL) and the reaction stirred at room temperature for 3 h. The solvent was removed under vacuum to afford crude methyl 4-(1-(O-(cyclohexylmethyl)-N—((S)-2-((S)-2,2-dimethylcyclopropane-1-carbonyl)-2,6-diazaspiro[3.4]octane-8-carbonyl)-L-threonyl)piperidin-4-yl)benzoate (932 mg, quant.) as a yellow oil which was used directly in the next step. LCMS m/z=651.7 [M+H]⁺.

Step 5: methyl 4-(1-(O-(cyclohexylmethyl)-N—((S)-6-(1-(cyclohexylmethyl)-1H-pyrazole-4-carbonyl)-2-((S)-2,2-dimethylcyclopropane-1-carbonyl)-2,6-diazaspiro[3.4]octane-8-carbonyl)-L-threonyl)piperidin-4-yl)benzoate: To a solution of 1-(cyclohexylmethyl)-1H-pyrazole-4-carboxylic acid (21 mg, 0.1 mmol) in DCM (2 mL) was added HATU (42 mg, 0.11 mmol) and DIEA (39 mg, 0.3 mmol) and the mixture stirred for 30 min. Methyl 4-(1-(O-(cyclohexylmethyl)-N—((S)-2-((S)-2,2-dimethylcyclopropane-1-carbonyl)-2,6-diazaspiro[3.4]octane-8-carbonyl)-L-threonyl)piperidin-4-yl)benzoate (70 mg, 0.1 mmol) was added and stirring continued overnight. The mixture was diluted with water (30 mL) and extracted with EtOAc (30 mL×3). The combined organic layers were washed with brine, dried over Na₂SO₄, filtered and concentrated. The residue obtained was purified by prep-TLC (eluent: DCM/MeOH=15/1) to afford methyl 4-(1-(O-(cyclohexylmethyl)-N—((S)-6-(1-(cyclohexylmethyl)-1H-pyrazole-4-carbonyl)-2-((S)-2,2-dimethylcyclopropane-1-carbonyl)-2,6-diazaspiro[3.4]octane-8-carbonyl)-L-threonyl)piperidin-4-yl)benzoate (100 mg, 77%) as white solid. LCMS m/z=842.2 [M+H]⁺; ¹H NMR (400 MHz, Chloroform-d) δ 7.98 (s, 2H), 7.81 (s, 1H), 7.78 (s, 1H), 7.26 (s, 2H), 6.95 (s, 1H), 5.07-4.98 (m, 1H), 4.83-4.72 (m, 1H), 4.18-4.08 (m, 2H), 4.03-3.87 (m, 9H), 3.76-3.66 (m, 3H), 3.38-3.10 (m, 6H), 1.97-1.86 (m, 3H), 1.65-1.58 (m, 4H), 1.49-1.46 (m, 10H), 1.44-1.42 (m, 7H), 1.17-1.12 (m, 12H), 0.95-0.90 (m, 2H).

Step 6: 4-(1-(O-(cyclohexylmethyl)-N—((S)-6-(1-(cyclohexylmethyl)-1H-pyrazole-4-carbonyl)-2-((S)-2,2-dimethylcyclopropane-1-carbonyl)-2,6-diazaspiro[3.4]octane-8-carbonyl)-L-threonyl)piperidin-4-yl)benzoic acid: To a solution of methyl 4-(1-(0-(cyclohexylmethyl)-N—((S)-6-(1-(cyclohexylmethyl)-1H-pyrazole-4-carbonyl)-2-((S)-2,2-dimethylcyclopropane-1-carbonyl)-2,6-diazaspiro[3.4]octane-8-carbonyl)-L-threonyl)piperidin-4-yl)benzoate (70 mg, 0.08 mmol) in a mixture of MeOH and water (1 mL/0.5 mL) was added NaOH (6.4 mg, 0.16 mmol). The reaction was stirred at room temperature for 4 h then was diluted with water (15 mL) and extracted with EtOAc (15 mL). The aqueous layer was collected, acidified to pH ~6 with 1M HCl then extracted with EtOAc (20 mL×3). The combined organic layers were washed with brine, dried over Na₂SO₄, filtered and concentrated to afford 4-(1-(O-(cyclohexylmethyl)-N—((S)-6-(1-(cyclohexylmethyl)-1H-pyrazole-4-carbonyl)-2-((S)-2,2-dimethylcyclopropane-1-carbonyl)-2,6-diazaspiro[3.4]octane-8-carbonyl)-L-threonyl)piperidin-4-yl)benzoic acid (29 mg, 42%) as a white solid. LCMS m/z=827.4 [M+H]⁺; ¹H NMR (400 MHz, Methanol-d₄) δ 8.16-8.05 (m, 1H), 7.99-7.85 (m, 3H), 7.37-7.27 (m, 2H), 5.01 (s, 1H), 4.68 (d, J=11.6 Hz, 1H), 4.40-4.13 (m, 3H), 4.11-3.74 (m, 8H), 3.44-3.34 (m, 1H), 3.28-3.17 (m, 1H), 2.97-2.87 (m, 1H), 2.83-2.73 (m, 1H), 2.01-1.81 (m, 3H), 1.81-1.49 (m, 14H), 1.26-1.11 (m, 15H), 1.08-0.83 (m, 8H), 0.82-0.72 (m, 1H).

Table I-12: The compounds listed in Table I-12 were synthesized from methyl 4-(1-(O-(cyclohexylmethyl)-N—((S)-2-((S)-2,2-dimethylcyclopropane-1-carbonyl)-2,6-diazaspiro[3.4]octane-8-carbonyl)-L-threonyl)piperidin-4-yl)benzoate according to the procedures outlined for I-30 using the appropriate commercially available reagents and/or intermediates described elsewhere.

TABLE I-12

| Ex- | | | |
|---|---|---|---|
| ample | | | |
| Num- | | | |
| ber | Compound | ¹HNMR | LCMS |
| I-29 | 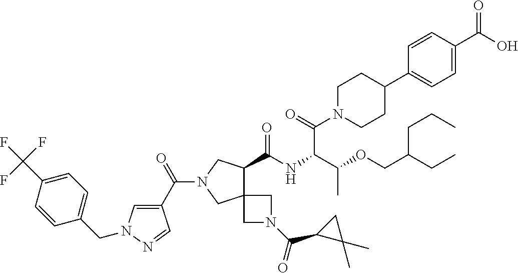 [SMILES: CCCC(CC)CO[C@H](C)[C@H](NC(=O)[C@@H]1CN(C(=O)c2cnn(Cc3ccc(C(F)(F)F)cc3)c2)CC12CN(C(=O)[C@H]1CC1(C)C)C2)C(=O)N1CCC(c2ccc(C(=O)O)cc2)CC1] | ¹H NMR (400 MHz, DMSO-d6) δ 8.59-8.34 (m, 2H), 7.93-7.81 (m, 3H), 7.72 (d, J = 7.9 Hz, 2H), 7.44 (d, J = 7.9 Hz, 2H), 7.37-7.29 (m, 2H), 5.47 (s, 2H), 5.02-4.82 (m, 1H), 4.65-4.46 (m, 1H), 4.28-3.49 (m, 11H), 3.45-3.36 (m, 1H),3.27-3.05 (m, 3H), 3.02-2.81 (m, 1H), 2.75- 2.58 (m, 1H), 1.92- 1.36 (m, 11H), 1.15- 1.01 (m, 11H), 0.91- 0.83 (m, 3H), 0.71-0.62 (m, 1H). | m/z = 889.3 [M + H]⁺ |

### Synthesis of methyl 4-((1R)-2-((2-oxabicyclo[2.2.2]octan-4-yl)methoxy)-1-(6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxamido)ethyl)benzoate (I-24); and 4-((1R)-2-((2-oxabicyclo[2.2.2]octan-4-yl)methoxy)-1-(6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxamido)ethyl)benzoic acid (I-23)

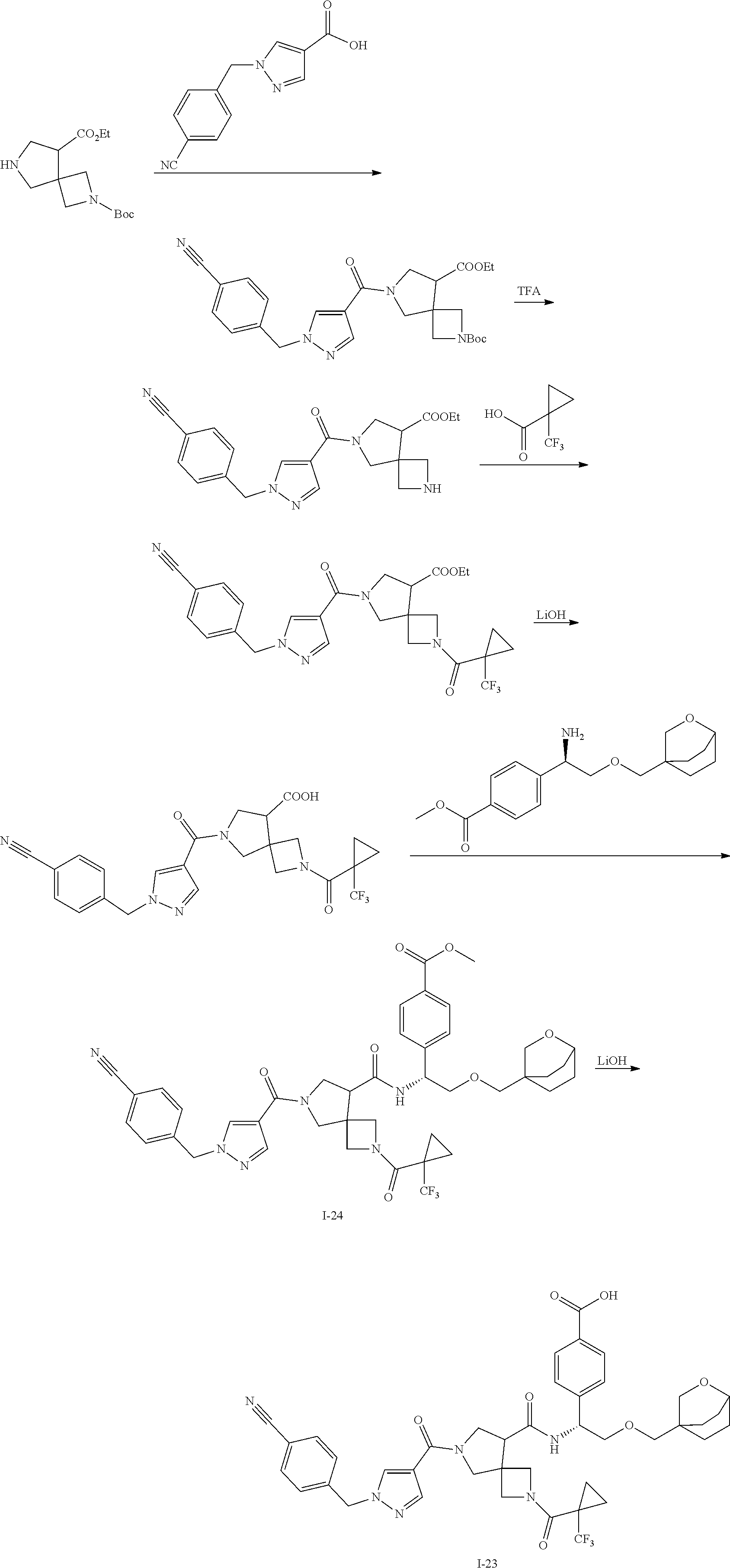

Step 1: 2-(tert-butyl) 8-ethyl 6-(1-(4-cyanobenzyl)-1H-pyrazole-4-carbonyl)-2,6-diazaspiro[3.4]octane-2,8-dicarboxylate: To a solution of 1-(4-cyanobenzyl)-1H-pyrazole-4-carboxylic acid (767.2 mg, 3.38 mmol) in DMF (15 mL) was added HATU (1.28 g, 3.38 mmol) and DIPEA (1.09 g, 8.44 mmol) and the mixture stirred at room temperature for 30 min. (2-(tert-butyl) 8-ethyl 2,6-diazaspiro[3.4]octane-2,8-dicarboxylate (800 mg, 2.81 mmol) was added and stirring continued for 2 h. The mixture was diluted with water (35 mL) and extracted with EtOAc (50 mL×3). The combined organic layers were washed with brine, dried over Na₂SO₄, filtered and concentrated. The residue was purified by R.P column to afford 2-(tert-butyl) 8-ethyl 6-(1-(4-cyanobenzyl)-1H-pyrazole-4-carbonyl)-2,6-diazaspiro[3.4]octane-2,8-dicarboxylate (1.1 g, 79%) as a white solid. LCMS m/z=438.1 [M-55+H]⁺; ¹H NMR (400 MHz, DMSO-d₆) δ 8.48-8.35 (m, 1H), 7.92-7.78 (m, 3H), 7.44-7.34 (m, 2H), 5.48 (s, 2H), 4.27-3.59 (m, 11H), 1.37 (s, 9H), 1.23-1.12 (m, 3H).

Step 2: ethyl 6-(1-(4-cyanobenzyl)-1H-pyrazole-4-carbonyl)-2,6-diazaspiro[3.4]octane-8-carboxylate: To a solution of 2-(tert-butyl) 8-ethyl 6-(1-(4-cyanobenzyl)-1H-pyrazole-4-carbonyl)-2,6-diazaspiro[3.4]octane-2,8-dicarboxylate (1.1 g, 2.2 mmol) in DCM (20 mL) was added TFA (10 mL) and the mixture stirred at room temperature for 4 hours. The solvent was removed under vacuum to afford ethyl 6-(1-(4-cyanobenzyl)-1H-pyrazole-4-carbonyl)-2,6-diazaspiro[3.4]octane-8-carboxylate (2.0 g, quant.) which was used directly in the next step. LCMS m/z=394.1 [M+H]⁺.

Step 3: ethyl 6-(1-(4-cyanobenzyl)-1H-pyrazole-4-carbonyl)-2-(1-(trifluoromethyl) cyclopropane-1-carbonyl)-2,6-diazaspiro[3.4]octane-8-carboxylate: To a solution of 1-(trifluoromethyl)cyclopropane-1-carboxylic acid (450 mg, 2.92 mmol) in DMF (10 mL) was added HATU (1.11 g, 2.92 mmol) and DIPEA (945 mg, 7.30 mmol) and the mixture stirred at room temperature for 30 min. Ethyl 6-(1-(4-cyanobenzyl)-1H-pyrazole-4-carbonyl)-2,6-diazaspiro[3.4]octane-8-carboxylate (957 mg, 2.43 mmol) was added and stirring continued for 2 h. The mixture was diluted with water (25 mL) and extracted with EtOAc (50 mL×3). The combined organic layers were washed with brine, dried over Na₂SO₄, filtered and concentrated. The residue was purified by column chromatography on silica gel (eluent: DCM:MeOH=40:1) to afford ethyl 6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxylate (800 mg, 62%) as a white solid. LCMS m/z=530.1 [M+H]⁺; ¹H NMR (400 MHz, Chloroform-d) δ 7.98 (s, 1H), 7.92-7.80 (m, 1H), 7.71-7.54 (m, 2H), 7.41-7.29 (m, 2H), 5.38 (s, 2H), 4.27-3.49 (m, 10H), 3.30-3.11 (m, 1H), 1.28 (t, J=7.2 Hz, 3H), 1.24-1.16 (m, 4H).

Step 4: 6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxylic acid: To a solution of ethyl 6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxylate (800 mg, 1.51 mmol) in a mixture of THE and water (20 mL/4 mL) was added LiOH (191 mg, 4.53 mmol). The reaction mixture was stirred at room temperature for 2 h, then was diluted with water (40 mL) and extracted with ether (50 mL). The aqueous layer was collected, acidified to pH ~2 with 1M HCl then extracted with EtOAc (200 mL×2). The combined organic layers were washed with brine, dried over Na₂SO₄, filtered and concentrated to afford 6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxylic acid (700 mg, 92%) as a white solid. LCMS m/z=502.1 [M+H]⁺; ¹HNMR (400 MHz, Chloroform-d) δ 7.98 (s, 1H), 7.84 (d, J=10.4 Hz, 1H), 7.64 (d, J=7.8 Hz, 2H), 7.35-7.28 (m, 2H), 5.38 (s, 2H), 4.14-3.47 (m, 8H), 3.31-3.15 (m, 1H), 1.23-1.17 (m, 4H).

Step 5: methyl 4-((1R)-2-((2-oxabicyclo[2.2.2]octan-4-yl)methoxy)-1-(6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxamido)ethyl)benzoate: To a solution of 6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxylic acid (100 mg, 0.199 mmol) in DMF (5 mL) was added HATU (84.0 mg, 0.219 mmol) and DIPEA (77 mg, 0.598 mmol) nd the mixture stirred at room temperature for 30 min. Methyl (R)-4-(2-((2-oxabicyclo[2.2.2]octan-4-yl)methoxy)-1-aminoethyl)benzoate (48.0 mg, 0.150 mmol) was added and stirring continued for 2 h. The mixture was diluted with water (10 mL) and extracted with EtOAc (25 mL×3). The combined organic layers were washed with brine, dried over Na₂SO₄, filtered and concentrated. The residue was purified by prep-TLC (eluent: DCM:MeOH=15:1 v/v) to afford methyl 4-((1R)-2-((2-oxabicyclo[2.2.2]octan-4-yl)methoxy)-1-(6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxamido)ethyl)benzoate (72 mg, 45%) as a white solid. LCMS m/z=803.2 [M+H]⁺; ¹H NMR (400 MHz, Methanol-d₄) δ 8.33-8.22 (m, 1H), 8.07-7.90 (m, 3H), 7.80-7.65 (m, 2H), 7.49-7.33 (m, 4H), 5.49 (s, 2H), 4.47-3.63 (m, 15H), 3.56-3.33 (m, 2H), 3.24-3.10 (m, 1H), 3.06-2.89 (m, 2H), 2.04-1.89 (m, 2H), 1.73-1.58 (m, 4H), 1.57-1.40 (m, 2H), 1.36-1.11 (m, 4H).

Step 6: 4-((1R)-2-((2-oxabicyclo[2.2.2]octan-4-yl)methoxy)-1-(6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxamido)ethyl)benzoic acid: To a solution of methyl 4-((1R)-2-((2-oxabicyclo[2.2.2]octan-4-yl)methoxy)-1-(6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxamido)ethyl)benzoate (62 mg, 0.077 mmol) in a mixture of THE and water (2 mL/0.2 mL) was added LiOH (11.0 mg, 0.231 mmol). The reaction mixture was stirred at room temperature for 5 h, then was diluted with water (10 mL) and extracted with ether (50 mL). The aqueous layer was collected, acidified to pH ~2 with 1M HCl and extracted with EtOAc (50 mL×2). The combined organic layers were washed with brine, dried over Na₂SO₄, filtered and concentrated. The residue was purified by prep-HPLC to afford 4-((1R)-2-((2-oxabicyclo[2.2.2]octan-4-yl)methoxy)-1-(6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxamido)ethyl)benzoic acid (20 mg, 33%) as a white solid. LCMS m/z=789.2 M+H]⁺; ¹H NMR (400 MHz, Methanol-d₄) δ 8.34-8.24 (m, 1H), 8.06-7.91 (m, 3H), 7.79-7.65 (m, 2H), 7.49-7.33 (m, 4H), 5.49 (s, 2H), 4.43-3.69 (m, 12H), 3.57-3.33 (m, 2H), 3.24-3.12 (m, 1H), 3.06-2.87 (m, 2H), 2.06-1.88 (m, 2H), 1.74-1.58 (m, 4H), 1.57-1.42 (m, 2H), 1.42-1.02 (m, 4H).

Table I-13: The compounds listed in Table I-13 were synthesized from 6-(1-(4-cyanobenzyl)-1H-pyrazole-4-carbonyl)-2-(1-(trifluoromethyl)cyclopropane-1-carbonyl)-2,6-diazaspiro[3.4]octane-8-carboxylic acid according to the procedures outlined for I-24 and I-23 using the appropriate commercially available reagents and/or intermediates described elsewhere.

TABLE I-13

| Example | | | |
|---|---|---|---|
| Number | Compound | ¹HNMR | LCMS |
| I-20 | 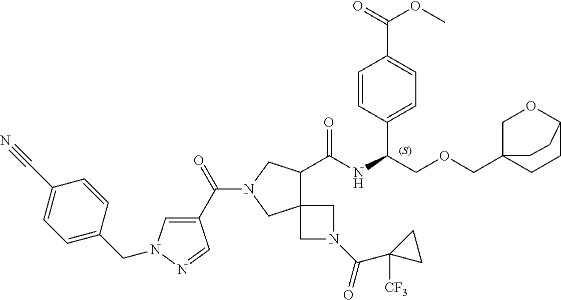 | ¹H NMR (400 MHz, Methanol-d₄) δ 8.35- 8.23 (m, 1H), 8.04-7.89 (m, 3H), 7.78-7.66 (m, 2H), 7.47-7.36 (m, 4H), 5.49 (s, 2H), 4.47-3.66 (m, 15H), 3.59-3.33 (m, 2H), 3.25-3.12 (m, 1H), 3.09-2.90 (m, 2H), 2.06- 1.84 (m, 2H), 1.76- 1.58 (m, 4H). | m/z = 803.2 [M + H]⁺ |
| I-19 | 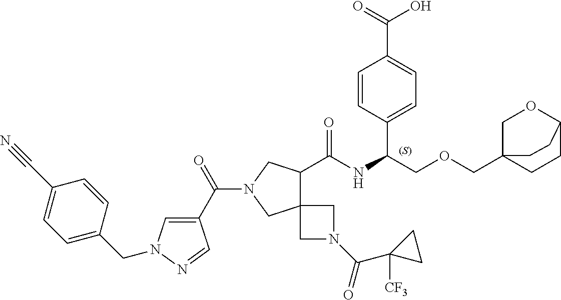 | ¹H NMR (400 MHz, Methanol-d₄) δ 8.33- 8.22 (m, 1H), 8.05-7.91 (m, 3H), 7.75-7.67 (m, 2H), 7.47-7.35 (m, 4H), 5.49 (s, 2H), 4.51-3.64 (m, 12H), 3.60-3.33 (m, 2H), 3.24-3.11 (m, 1H), 3.10-2.84 (m, 2H), 2.07- 1.89 (m, 2H), 1.80- 1.37 (m, 6H), 1.27-1.16 (m, 4H). | m/z = 789.2 [M + H]⁺ |

### Building Blocks:

### Synthesis of methyl 4-(piperidin-4-yl)benzoate

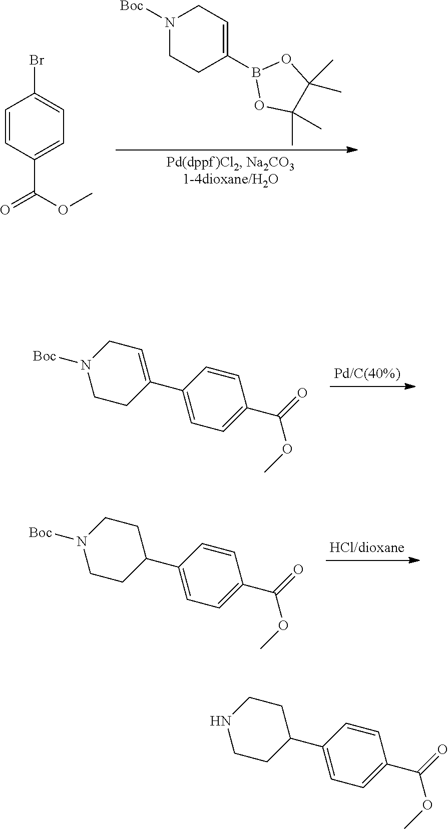 [SMILES: COC(=O)c1ccc(Br)cc1 ; CC(C)(C)OC(=O)N1CC=C(B2OC(C)(C)C(C)(C)O2)CC1 ; COC(=O)c1ccc(C2=CCN(C(=O)OC(C)(C)C)CC2)cc1 ; COC(=O)c1ccc(C2CCN(C(=O)OC(C)(C)C)CC2)cc1 ; COC(=O)c1ccc(C2CCNCC2)cc1]

Step 1: tert-butyl 4-(4-(methoxycarbonyl)phenyl)-3,6-dihydropyridine-1 (2H)-carboxylate: To a solution of methyl 4-bromobenzoate (500 mg, 2.3 mmol) in a mixture of dioxane (8 mL) and water (2 mL) was added Pd(dppf)Cl₂ (168 mg, 0.23 mmol), tert-butyl 4-(4,4,5,5-tetramethyl-1,3,2-dioxaborolan-2-yl)-3,6-dihydropyridine-1 (2H)-carboxylate (797 mg, 2.3 mmol) and Na₂CO₃ (731 mg, 6.9 mmol). The reaction was heated under N₂ atmosphere at 90° C. overnight then was diluted with water (30 mL) and extracted with EtOAc (80 mL×3). The combined organic layers were washed with brine, dried over Na₂SO₄, filtered and concentrated. The residue was purified by column chromatography on silica gel (eluent: Pet. Ether:EtOAc=20:1) to afford tert-butyl 4-(4-(methoxycarbonyl)phenyl)-3,6-dihydropyridine-1 (2H)-carboxylate (0.87 g, 38%) as a white solid. LCMS m/z=318.3 [M+H]⁺; ¹H NMR (400 MHz, DMSO-d₆) δ 7.95-7.90 (m, 2H), 7.58 (d, J=8.4 Hz, 2H), 6.33 (s, 1H), 4.03 (t, J=3.6 Hz, 2H), 3.84 (s, 3H), 3.54 (s, 2H), 2.50 (s, 2H), 1.43 (s, 9H).

Step 2: tert-butyl 4-(4-(methoxycarbonyl)phenyl)piperidine-1-carboxylate: To a solution of tert-butyl 4-(4-(methoxycarbonyl)phenyl)-3,6-dihydropyridine-1 (2H)-carboxylate (2 g, 6.3 mmol) in MeOH (12 mL) was added 10% Pd/C (800 mg) and the reaction stirred under H₂ atmosphere for 2 hours. The catalyst was removed by filtration through Celite and the filtrate concentrated to afford tert-butyl 4-(4-(methoxycarbonyl)phenyl)piperidine-1-carboxylate (1.8 g, 92%) as a colorless oil. LCMS m/z=320.1 [M+H]⁺; ¹H NMR (400 MHz, DMSO-d₆) δ 7.89 (d, J=8.2 Hz, 2H), 7.39 (d, J=8.2 Hz, 2H), 4.05 (dd, J=20.2, 9.9 Hz, 2H), 3.83 (s, 3H), 2.78 (dt, J=6.8, 3.5 Hz, 3H), 1.74 (s, 2H), 1.51 (td, J=12.6, 4.2 Hz, 2H), 1.41 (s, 9H).

Step 3: methyl 4-(piperidin-4-yl)benzoate: To a solution of tert-butyl 4-(4-(methoxycarbonyl)phenyl)piperidine-1-carboxylate (200 mg, 0.63 mmol) in DCM (2 mL) was added a solution of HCl in dioxane (4M, 2 mL). The reaction was stirred at room temperature for 2 h then the solvent was removed under vacuum to afford methyl 4-(piperidin-4-yl)benzoate (137 mg, 100%) which was used directly in the next step. LCMS m/z=184.1 [M+H]⁺.

### Synthesis of dimethyl (2-aminoethyl)phosphonate

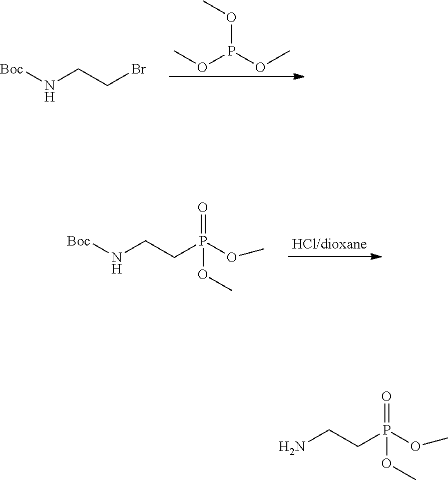

Step 1: tert-butyl (2-(dimethoxyphosphoryl)ethyl)carbamate: A solution of tert-butyl (2-bromoethyl)carbamate (1.0 g, 4.46 mmol) in trimethyl phosphite (13 mL) was heated at 120° C. under a N₂ atmosphere for 48 h. The mixture was concentrated to afford tert-butyl (2-(dimethoxyphosphoryl)ethyl)carbamate (1.1 g, 98%) which was used directly in the next step. ¹H NMR (400 MHz, DMSO-d₆) δ 7.40 (s, 1H), 3.67 (d, J=11.0 Hz, 2H), 3.52 (d, J=11.0 Hz, 6H), 1.99-1.85 (m, 2H), 1.37 (s, 9H).

Step 2: dimethyl (2-aminoethyl)phosphonate: To a solution of tert-butyl (2-(dimethoxyphosphoryl)ethyl)carbamate (20 mg, 0.08 mmol) in DCM (2 mL) was added a solution of HCl in dioxane (1.0 mL, 4.0 M in dioxane). The reaction mixture was stirred at room temperature for 2 h then the solvent was removed under vacuum to afford dimethyl (2-aminoethyl)phosphonate (12 mg, 100%) which was used directly in the next step.

### Synthesis of methyl O-((2-oxabicyclo[2.2.2]octan-4-yl)methyl)-L-threoninate

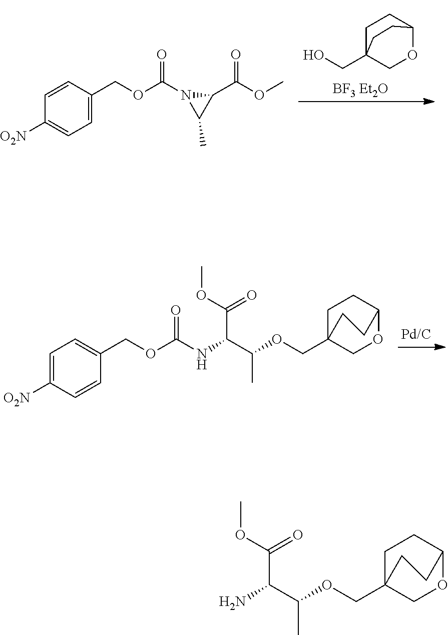

Step 1: methyl O-((2-oxabicyclo[2.2.2]octan-4-yl)methyl)-N-(((4-nitrobenzyl)oxy)carbonyl)-L-threoninate: To a solution of 2-methyl 1-(4-nitrobenzyl) (2S,3S)-3-methylaziridine-1,2-dicarboxylate (500 mg, 1.7 mmol) in DCM (0.5 mL) was added (2-oxabicyclo[2.2.2]octan-4-yl)methanol (242 mg, 1.7 mmol) and BF₃·Et₂O (724 mg, 5.1 mmol). The mixture was stirred at room temperature for 2 h then was concentrated under vacuum and the residue purified by silica gel column (Pet.ether/EtOAc=4/1, v/v) to afford methyl O-((2-oxabicyclo[2.2.2]octan-4-yl)methyl)-N-(((4-nitrobenzyl)oxy)carbonyl)-L-threoninate (100 mg, 13%) as a yellow oil. ¹H NMR (400 MHz, DMSO-d₆) δ 8.25 (d, J=8.7 Hz, 2H), 7.66-7.55 (m, 3H), 5.21 (s, 2H), 4.24-4.20 (m, 1H), 3.83-3.76 (m, 1H), 3.64-3.58 (m, 4H), 3.56-3.33 (m, 4H), 1.86-1.43 (m, 6H), 1.39-1.31 (m, 2H), 1.09-1.05 (m, 3H).

Step 2: methyl O-((2-oxabicyclo[2.2.2]octan-4-yl)methyl)-L-threoninate: To a solution of methyl O-((2-oxabicyclo[2.2.2]octan-4-yl)methyl)-N-(((4-nitrobenzyl)oxy)carbonyl)-L-threoninate (1.03 g, 2.36 mmol) in MeOH (10 mL) was added Pd/C (412 mg, 3.87 mmol) and the reaction heated at 50° C. for 2 h under a hydrogen atmosphere. The catalyst was removed by filtration through Celite and the filtrate concentrated to afford methyl O-((2-oxabicyclo[2.2.2]octan-4-yl)methyl)-L-threoninate (600 mg, 98%) as a colorless oil. LCMS m/z=258.2 [M+H]⁺.

### Synthesis of piperidine-4-carbonitrile

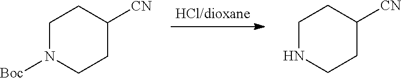 [SMILES: CC(C)(C)OC(=O)N1CCC(C#N)CC1 ; N#CC1CCNCC1]

To a solution of tert-butyl 4-cyanopiperidine-1-carboxylate (25 mg, 0.119 mmol) in DCM (1 mL) was added a solution of HCl in dioxane (4M, 0.5 mL) and the reaction stirred at room temperature for 2 h. The solvent was removed under vacuum to afford piperidine-4-carbonitrile as a white solid. LCMS m/z=110.6 [M+H]⁺.

### Synthesis of 1-(4-(trifluoromethyl)benzyl)-1H-pyrazole-4-carboxylic acid

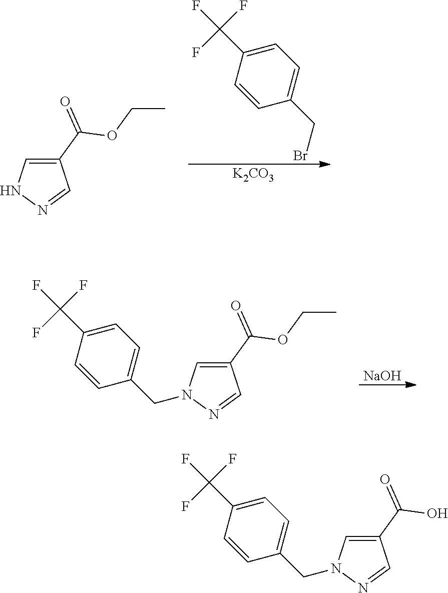

Step 1: ethyl 1-(4-(trifluoromethyl)benzyl)-1H-pyrazole-4-carboxylate: To a solution of ethyl 1H-pyrazole-4-carboxylate (13.0 g, 93.0 mmol) in ACN (100 mL) was added 1-(bromomethyl)-4-(trifluoromethyl)benzene (24.0 g, 101.3 mmol) and K₂CO₃ (25.6 g, 186.0 mmol) and the reaction stirred at room temperature overnight. The mixture was diluted with water (100 mL) and extracted with EtOAc (200 mL×3). The combined organic layers were washed with brine, dried over Na₂SO₄, filtered and concentrated to afford ethyl 1-(4-(trifluoromethyl)benzyl)-1H-pyrazole-4-carboxylate (27.7 g, 100%) as a white solid. ¹H NMR (400 MHz, DMSO-d6) δ 8.53 (s, 1H), 7.90 (s, 1H), 7.72 (d, J=8.0 Hz, 2H), 7.45 (d, J=8.0 Hz, 2H), 5.49 (s, 2H), 4.21 (q, J=7.0 Hz, 2H), 1.26 (t, J=7.2 Hz, 3H).

Step 2: 1-(4-(trifluoromethyl)benzyl)-1H-pyrazole-4-carboxylic acid: To a solution of ethyl 1-(4-(trifluoromethyl)benzyl)-1H-pyrazole-4-carboxylate (27.7 g, 92.8 mmol) in a mixture of THE and water and EtOH (160 mL/40 mL/40 mL) was added NaOH (11.1 g, 278 mmol) and the reaction stirred at room temperature overnight. The mixture was diluted with water (100 mL) and extracted with EtOAc (50 mL×3). The aqueous layer was collected, acidified with to pH ~2 1M HCl and extracted with EtOAc (50 mL×3). The combined organic layers were washed with brine, dried over Na₂SO₄, filtered and concentrated to afford 1-(4-(trifluoromethyl)benzyl)-1H-pyrazole-4-carboxylic acid (21.0 g, 84%) as a white solid. LCMS m/z=271.2 [M+H]⁺; ¹H NMR (400 MHz, DMSO-d6) δ 12.37 (s, 1H), 8.44 (s, 1H), 7.85 (s, 1H), 7.72 (d, J=8.2 Hz, 2H), 7.45 (d, J=8.0 Hz, 2H), 5.48 (s, 2H).

### Synthesis of 1-(2-(methoxycarbonyl)benzyl)-1H-pyrazole-4-carboxylic acid

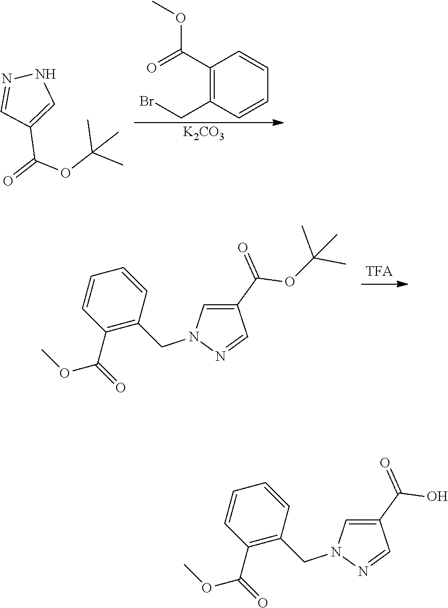

Step 1: tert-butyl 1-(2-(methoxycarbonyl)benzyl)-1H-pyrazole-4-carboxylate: To a solution of tert-butyl 1H-pyrazole-4-carboxylate (1 g, 5.945 mmol) in ACN (15 mL) were added methyl 2-(bromomethyl)benzoate (1.36 g, 5.945 mmol) and K₂CO₃ (2.465 g, 17.835 mmol) and the reaction stirred at room temperature overnight. The reaction was quenched with water and extracted with EtOAc (25 mL×3). The combined organic phases were washed with brine, dried over Na₂SO₄ filtered and concentrated to afford tert-butyl 1-(2-(methoxycarbonyl)benzyl)-1H-pyrazole-4-carboxylate (1.9 g, 100%) as a colorless oil. LCMS m/z=317.15 [M+H]⁺; ¹H NMR (400 MHz, DMSO-d₆) & 8.30 (s, 1H), 7.91 (dd, J=1.6 Hz, J₂=1.2 Hz, 1H), 7.81 (s, 1H), 7.58-7.54 (m, 1H), 7.47-7.43 (m, 1H), 6.88-6.86 (m, 1H), 5.71 (s, 2H), 3.86 (s, 3H), 1.49 (s, 9H).

Step 2: 1-(2-(methoxycarbonyl)benzyl)-1H-pyrazole-4-carboxylic acid: To a solution of tert-butyl 1-(2-(methoxycarbonyl)benzyl)-1H-pyrazole-4-carboxylate in DCM (1 mL) was added TFA (0.5 mL) and the reaction stirred at room temperature for 2 h. The solvent was removed under vacuum to provide 1-(2-(methoxycarbonyl)benzyl)-1H-pyrazole-4-carboxylic acid as a white solid. LCMS m/z=261.2 [M+H]⁺.

Table I-14: The compounds listed in Table I-14 were synthesized according to the procedure outlined for 4-nitrobenzyl ((2S,3R)-3-((2-oxabicyclo[2.2.2]octan-4-yl)methoxy)-1-oxo-1-(4-(4-(trifluoromethyl) benzyl)piperazin-1-yl)butan-2-yl)carbamate using the appropriate commercially available reagents.

TABLE I-14

| Compound | LCMS | ¹H NMR |
|---|---|---|
| methyl 4-(1-(O-((2-oxabicyclo[2.2.2]octan-4- | LCMS m/z = | |
| yl)methyl)-N-(((4-nitrobenzyl)oxy)carbonyl)-L- | 692.2 [M + H]⁺ | |
| threonyl)piperidin-4-yl)-2- | | |
| (trifluoromethyl)benzoate | | |
| 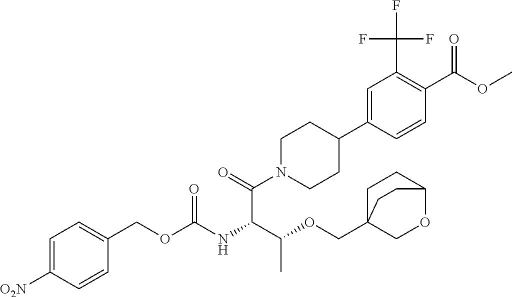 [SMILES: COC(=O)c1ccc(C2CCN(C(=O)[C@@H](NC(=O)OCc3ccc([N+](=O)[O-])cc3)[C@@H](C)OCC34CCC(CC3)OC4)CC2)cc1C(F)(F)F] | | |
| methyl 2-(4-(1-(O-((2-oxabicyclo[2.2.2]octan-4- | LCMS m/z = | ¹H NMR (400 MHz, |
| yl)methyl)-N-(((4-nitrobenzyl)oxy)carbonyl)-L- | 750.3 [M + H]⁺ | DMSO-d6) δ 8.23 (d, J = |
| threonyl)piperidin-4-yl)-2- | | 8.2 Hz, 1H), 8.15 (d, J = 8.6 |
| (trifluoromethyl)phenoxy)-2-methylpropanoate | | Hz, 1H), 7.67-7.34 (m, |
| 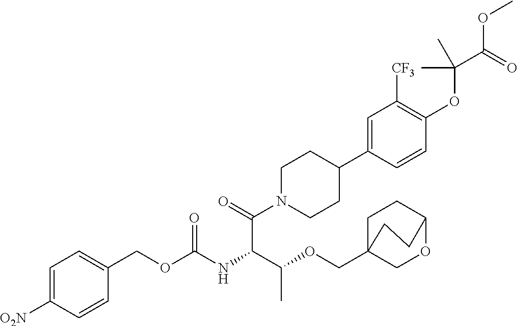 [SMILES: COC(=O)C(C)(C)Oc1ccc(C2CCN(C(=O)[C@@H](NC(=O)OCc3ccc([N+](=O)[O-])cc3)[C@@H](C)OCC34CCC(CC3)OC4)CC2)cc1C(F)(F)F] | | 5H), 6.80-6.73 (m, 1H), 5.28-5.11 (m, 2H), 4.49 (d, 2H), 4.22-4.08 (m, 1H), 3.73 (s, 3H), 3.64- 3.46 (m, 4H), 3.20-3.04 (m, 2H), 3.01-2.94 (m, 1H), 2.90-2.76 (m, 1H), 2.68-2.57 (m, 1H), 1.90- 1.74 (m, 3H), 1.73-1.61 (m, 1H), 1.53 (s, 6H), 1.50 (s, 2H), 1.40-1.31 (m, 3H), 1.23 (s, 3H), 1.10- 0.99 (m, 3H). |
| methyl 4-(1-(O-((2-oxabicyclo[2.2.2]octan-4- | LCMS m/z = | ¹H NMR (400 MHz, |
| yl)methyl)-N-(((4-nitrobenzyl)oxy)carbonyl)-L- | 624.2 [M + H]⁺ | DMSO-d6) δ 8.33-8.10 |
| threonyl)piperidin-4-yl)benzoate | | (m, 1H), 7.96-7.84 (m, |
| 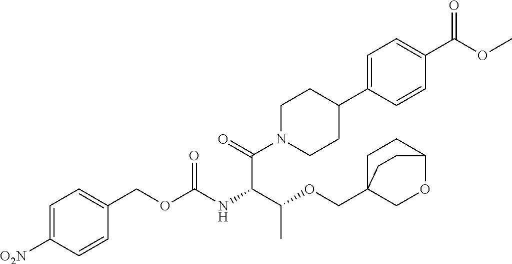 [SMILES: COC(=O)c1ccc(C2CCN(C(=O)[C@@H](NC(=O)OCc3ccc([N+](=O)[O-])cc3)[C@@H](C)OCC34CCC(CC3)OC4)CC2)cc1] | | 2H), 7.75-7.50 (m, 2H), 7.48-7.28 (m, 2H), 5.28- 5.11 (m, 1H), 4.66-4.44 (m, 2H), 4.26-4.10 (m, 1H), 3.84 (s, 3H), 3.65- 3.41 (m, 4H), 3.39-3.34 (m, 1H), 3.28-3.07 (m, 2H), 3.02-2.86 (m, 1H), 2.72-2.61 (m, 1H), 2.05- 1.95 (m, 1H), 1.89-1.66 (m, 3H), 1.63-1.42 (m, 5H), 1.40-1.19 (m, 5H), 1.12-1.01 (m, 2H), 0.91- 0.83 (m, 1H). |

The compounds listed in Table I-14 cont. were synthesized according to the
procedure outlined for (2S,3R)-2-amino-3-(2-cyclohexylethoxy)-N-methylbutanamide using the
appropriate commercially available reagents.

| Compound | LCMS |
|---|---|
| methyl 4-(1-(O-((2-oxabicyclo[2.2.2]octan-4-yl)methyl)- | LCMS m/z = 512.99 [M + H]⁺ |
| L-threonyl)piperidin-4-yl)-2-(trifluoromethyl)benzoate | |
| 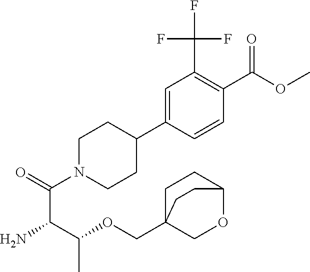 [SMILES: COC(=O)c1ccc(C2CCN(C(=O)[C@@H](N)[C@@H](C)OCC34CCC(CC3)OC4)CC2)cc1C(F)(F)F] | |
| methyl 2-(4-(1-(O-((2-oxabicyclo[2.2.2]octan-4- | LCMS m/z = 571.3 [M + H]⁺ |
| yl)methyl)-L-threonyl)piperidin-4-yl)-2- | |
| (trifluoromethyl)phenoxy)-2-methylpropanoate | |
| 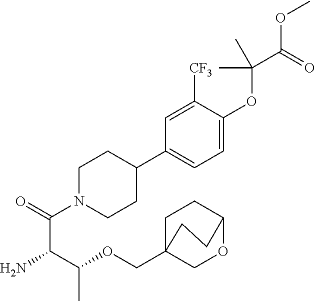 [SMILES: COC(=O)C(C)(C)Oc1ccc(C2CCN(C(=O)[C@@H](N)[C@@H](C)OCC34CCC(CC3)OC4)CC2)cc1C(F)(F)F] | |
| methyl 4-(1-(O-((2-oxabicyclo[2.2.2]octan-4-yl)methyl)- | LCMS m/z = 445.3 [M + H]⁺ |
| L-threonyl)piperidin-4-yl)benzoate | |
| 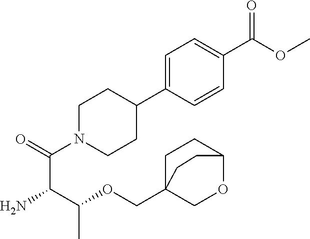 [SMILES: COC(=O)c1ccc(C2CCN(C(=O)[C@@H](N)[C@@H](C)OCC34CCC(CC3)OC4)CC2)cc1] | |

### Synthesis of Methyl 2-(piperidin-4-yl)benzoate

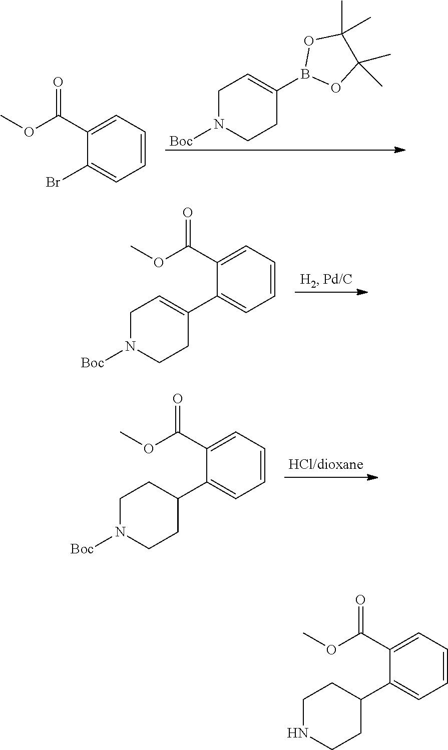

Step 1: tert-butyl 4-(2-(methoxycarbonyl)phenyl)-3,6-dihydropyridine-1 (2H)-carboxylate: To a solution of methyl 2-bromobenzoate (1 g, 4.65 mmol) in dioxane (8 mL) under a N₂ atmosphere was added tert-butyl 4-(4,4,5,5-tetramethyl-1,3,2-dioxaborolan-2-yl)-3,6-dihydropyridine-1 (2H)-carboxylate (1.73 g, 5.6 mmol), Pd(dppf)Cl₂ (169 mg, 0.2 mmol) and K₂CO₃ (1.93 g, 14 mmol) and the mixture heated at 100° C. for 2 h. The reaction was cooled to room temperature, diluted with water (60 mL) and extracted with EtOAc (80 mL×3). The combined organic layers were washed with brine, dried over Na₂SO₄, filtered and concentrated. The residue obtained was purified by column chromatography (Petroleum ether:EtOAc=30:1 to 27:1) to afford tert-butyl 4-(2-(methoxycarbonyl)phenyl)-3,6-dihydropyridine-1 (2H)-carboxylate (800 mg, 76%) as yellow oil. LCMS m/z=218.1 [M+H-100]⁺; ¹H NMR (400 MHz, DMSO-d6) δ 7.71 (s, 1H), 7.53 (d, J=7.8 Hz, 1H), 7.40 (s, 1H), 7.28 (d, J=7.6 Hz, 1H), 5.52-5.42 (m, 1H), 3.92 (s, 2H), 3.77 (s, 1H), 3.70 (s, 1H), 3.51 (d, J=5.8 Hz, 2H), 3.26 (s, 1H), 2.23 (d, J=17.0 Hz, 2H), 1.43 (s, 9H).

Step 2: tert-butyl 4-(2-(methoxycarbonyl)phenyl)piperidine-1-carboxylate: To a solution of tert-butyl 4-(2-(methoxycarbonyl)phenyl)-3,6-dihydropyridine-1 (2H)-carboxylate (700 mg, 2.2 mmol) in MeOH (8 mL) was added Pd/C (40%, 280 mg) and the reaction heated under a H₂ atmosphere at 50° C. for 2 h. The catalyst was removed by filtration through Celite and the filtrate concentrated to afford tert-butyl 4-(2-(methoxycarbonyl)phenyl)piperidine-1-carboxylate (600 mg, 85%) as a colorless oil. LCMS m/z=220.2 [M+H-100]⁺; ¹H NMR (400 MHz, Chloroform-d) δ 7.79 (dd, J=7.8, 1.5 Hz, 1H), 7.46 (td, J=7.6, 1.5 Hz, 1H), 7.35 (dd, J=8.0, 1.3 Hz, 1H), 7.26-7.23 (m, 1H), 4.24 (s, 2H), 3.90 (s, 3H), 3.54 (s, 1H), 2.83 (t, J=12.8 Hz, 2H), 1.83 (d, J=12.6 Hz, 2H), 1.61 (dd, J=12.6, 4.1 Hz, 2H), 1.48 (s, 9H).

Step 3: methyl 2-(piperidin-4-yl)benzoate: To a solution of tert-butyl 4-(2-(methoxycarbonyl)phenyl)piperidine-1-carboxylate (200 mg, 0.6 mmol) in DCM (2 mL) was added a solution of HCl in 1,4-dioxane (4 M, 2 mL). The reaction mixture was stirred at room temperature for 2 h then the solvent was removed under vacuum to afford methyl 2-(piperidin-4-yl)benzoate (137 mg) as a white solid. LCMS m/z=220.1 [M+H]⁺; ¹H NMR (400 MHz, DMSO-d6) δ 8.83 (s, 1H), 7.72 (dd, J=7.8, 1.4 Hz, 1H), 7.59 (d, J=1.6 Hz, 1H), 7.41-7.33 (m, 2H), 3.85 (s, 3H), 3.49 (d, J=5.4 Hz, 1H), 3.38 (s, 2H), 3.03-2.95 (m, 2H), 1.89 (q, J=4.0 Hz, 4H).

### Synthesis of Tert-butyl 4-(4-((1-methoxy-2-methyl-1-oxopropan-2-yl)oxy)-3-(trifluoromethyl)phenyl)piperidine-1-carboxylate

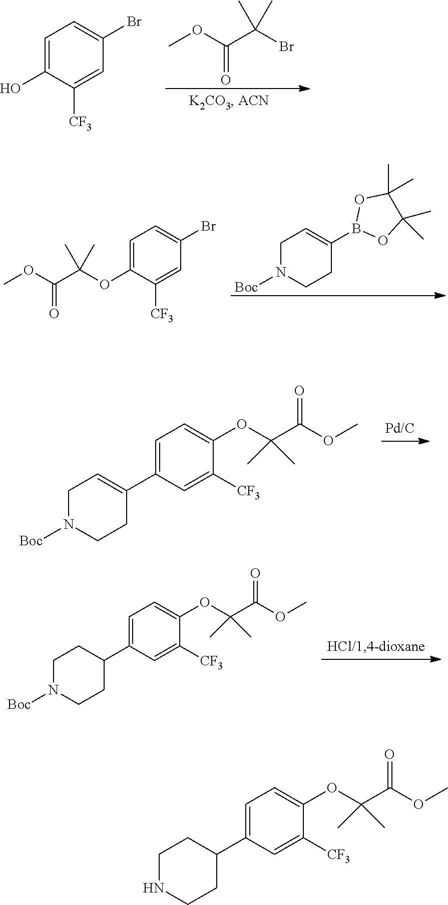

Step 1: methyl 2-(4-bromo-2-(trifluoromethyl)phenoxy)-2-methylpropanoate: To a solution of 4-bromo-2-(trifluoromethyl)phenol (3.0 g, 12.45 mmol) in ACN (30 mL) was added methyl 2-bromo-2-methylpropanoate (2.7 g, 14.90 mmol), K₂CO₃ (5.16 g, 37.34 mmol) and the reaction heated at 80° C. overnight. The mixture was diluted with water (50 mL) and extracted with EtOAc (50 ml×3). The combined organic layers were washed with brine, dried over Na₂SO₄, filtered and concentrated. The residue obtained was purified by column chromatography on silica gel (eluent: Pet. Ether:EtOAc=6:1) to afford methyl 2-(4-bromo-2-(trifluoromethyl)phenoxy)-2-methylpropanoate (2.8 g, 66%) as a yellow oil. ¹H NMR (400 MHz, DMSO-d₆) δ 7.78 (d, J=2.4 Hz, 1H), 7.76-7.72 (m, 1H), 6.83 (d, J=8.8 Hz, 1H), 3.73 (s, 3H), 1.56 (s, 6H).

Step 2: tert-butyl 4-(4-((1-methoxy-2-methyl-1-oxopropan-2-yl)oxy)-3-(trifluoromethyl)phenyl)-3,6-dihydropyridine-1 (2H)-carboxylate: To a solution of methyl 2-(4-bromo-2-(trifluoromethyl)phenoxy)-2-methylpropanoate (2.60 g, 7.62 mmol), Na₂CO₃ (2.42 g, 22.87 mmol) and Pd(dppf)Cl₂ (0.55 g, 0.76 mmol) in a mixture of dioxane (20 mL) and water (5 mL) was added tert-butyl 4-(4,4,5,5-tetramethyl-1,3,2-dioxaborolan-2-yl)-3,6-dihydropyridine-1 (2H)-carboxylate (2.36 g, 7.62 mmol) and the reaction heated at 90° C. under a N₂ atmosphere overnight. The mixture was diluted with water (50 mL) and extracted with EtOAc (50 mL×3). The combined organic layers were washed with brine, dried over Na₂SO₄, filtered and concentrated. The residue obtained was purified by column chromatography on silica gel to afford tert-butyl 4-(4-((1-methoxy-2-methyl-1-oxopropan-2-yl)oxy)-3-(trifluoromethyl)phenyl)-3,6-dihydropyridine-1 (2H)-carboxylate (2.80 mg, 83%) as a yellow oil. ¹H NMR (400 MHz, DMSO-d₆) δ 7.64-7.58 (m, 2H), 6.81 (d, J=9.2 Hz, 1H), 6.15 (s, 1H), 3.98 (d, 2H), 3.74 (s, 3H), 3.55-3.49 (m, 2H), 2.44 (d, J=5.6 Hz, 2H), 1.56 (s, 6H), 1.42 (s, 9H).

Step 3: tert-butyl 4-(4-((1-methoxy-2-methyl-1-oxopropan-2-yl)oxy)-3-(trifluoromethyl)phenyl) piperidine-1-carboxylate: To a solution of tert-butyl 4-(4-((1-methoxy-2-methyl-1-oxopropan-2-yl)oxy)-3-(trifluoromethyl)phenyl)-3,6-dihydropyridine-1 (2H)-carboxylate (2.4 g, 5.42 mmol) in MeOH (5 mL) was added 50% Pd/C (1.2 g) and the reaction stirred under a H₂ atmosphere for 15 h. The catalyst was removed by filtration through Celite and the filtrate concentrated to afford tert-butyl 4-(4-((1-methoxy-2-methyl-1-oxopropan-2-yl)oxy)-3-(trifluoromethyl)phenyl)piperidine-1-carboxylate (2.35 g, 97%) which was used without further purification. LCMS m/z=444.1 [M−H]⁻; ¹H NMR (400 MHz, DMSO-d₆) δ 7.49-7.41 (m, 2H), 6.76 (d, J=8.6 Hz, 1H), 4.11-4.04 (m, 2H), 3.74 (s, 3H), 2.85-2.66 (m, 3H), 1.77-1.70 (m, 2H), 1.54 (s, 6H), 1.51-1.44 (m, 2H), 1.41 (s, 9H).

Step 4: methyl 2-methyl-2-(4-(piperidin-4-yl)-2-(trifluoromethyl)phenoxy)propanoate: To a solution of tert-butyl 4-(4-((1-methoxy-2-methyl-1-oxopropan-2-yl)oxy)-3-(trifluoromethyl)phenyl) piperidine-1-carboxylate (150 mg, 0.36 mmol) in DCM (1 mL) was added a solution of HCl in dioxane (1 mL, 4.0 M in dioxane) and the reaction stirred at room temperature for 30 min. The solvent was removed under vacuum to afford methyl 2-methyl-2-(4-(piperidin-4-yl)-2-(trifluoromethyl)phenoxy)propanoate (124 mg, 100%) as a white solid. LCMS m/z=346.2 [M+H]⁺.

### Synthesis of Methyl 4-(piperidin-4-yl)benzoate

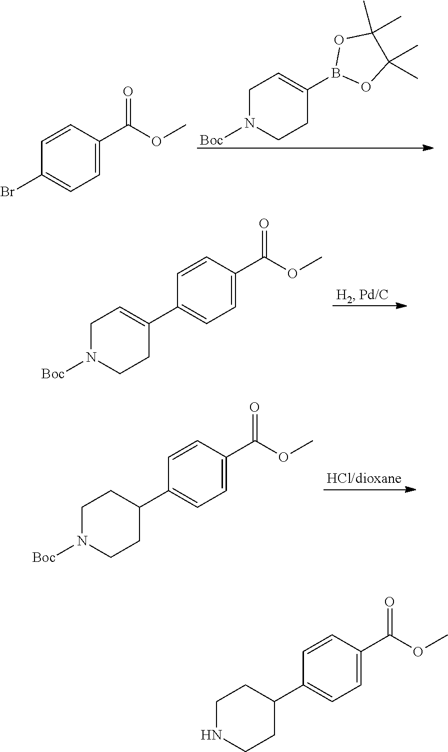

Step 1: tert-butyl 4-(4-(methoxycarbonyl)phenyl)-3,6-dihydropyridine-1 (2H)-carboxylate: To a solution of methyl 4-bromobenzoate (1.0 g, 4.65 mmol) in a mixture of 1,4-dioxane (10 mL) and water (2.5 mL) was added tert-butyl 4-(4,4,5,5-tetramethyl-1,3,2-dioxaborolan-2-yl)-3,6-dihydropyridine-1 (2H)-carboxylate (1.72 g, 5.58 mmol), K₂CO₃ (1.93 g, 13.95 mmol) and Pd(dppf)Cl₂ (170 mg, 0.23 mol). The reaction was heated at 100° C. under N₂ atmosphere overnight then was diluted with water (50 mL) and extracted with EtOAc (100 mL×3). The combined organic layers were dried over Na₂SO₄, filtered and concentrated. The residue was purified by column chromatography on silica gel (eluent: Pet. Ether:EtOAc=3:1, v/v) to afford tert-butyl 4-(4-(methoxycarbonyl)phenyl)-3,6-dihydropyridine-1 (2H)-carboxylate (1.02 g, 71%) as a white solid. ¹H NMR (400 MHz, DMSO-d₆) δ 7.92 (d, 2H), 7.58 (d, 2H), 6.33 (s, 1H), 4.03 (s, 2H), 3.84 (s, 3H), 3.54 (t, J=5.7 Hz, 2H), 2.50-2.45 (m, 2H), 1.43 (s, 9H).

Step 2: tert-butyl 4-(4-(methoxycarbonyl)phenyl)piperidine-1-carboxylate: To a solution of tert-butyl 4-(4-(methoxycarbonyl)phenyl)-3,6-dihydropyridine-1 (2H)-carboxylate (250 mg, 0.79 mmol) in MeOH (3 mL) was added 10% Pd/C (125 mg, 50%) and the reaction heated at 50° C. under a H₂ atmosphere overnight. The catalyst was removed by filtration through Celite and the filtrate concentrated to afford the tert-butyl 4-(4-(methoxycarbonyl)phenyl)piperidine-1-carboxylate (190 mg crude, 76%) as a white solid. LCMS m/z=263.7 [M+H-56]⁺; ¹H NMR (400 MHz, DMSO-d₆) δ 7.89 (d, 2H), 7.40 (d, J=8.2 Hz, 2H), 4.07 (d, J=13.1 Hz, 2H), 3.83 (s, 3H), 2.91-2.70 (m, 3H), 1.76 (d, J=12.8 Hz, 2H), 1.56-1.46 (m, 2H), 1.41 (s, 9H).

Step 3: methyl 4-(piperidin-4-yl)benzoate: To a solution of tert-butyl 4-(4-(methoxycarbonyl)phenyl)piperidine-1-carboxylate (78 mg, 0.30 mmol) in DCM (2 mL) was added 4M HCl in dioxane (1 mL). The mixture was stirred at room temperature for 2 h then the solvent was removed to afford methyl 4-(piperidin-4-yl)benzoate (53 mg, 100%) as a yellow oil. LCMS m/z=220.2 [M+H]⁺.

### Synthesis of Methyl 4-(1-(O-(cyclohexylmethyl)-L-threonyl)piperidin-4-yl)benzoate

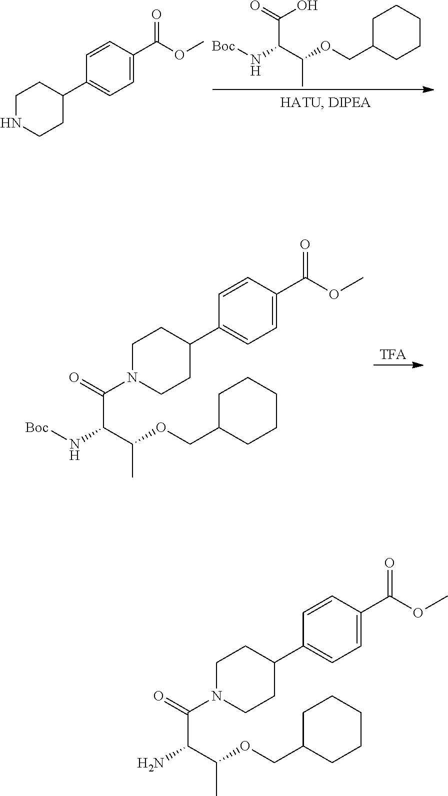

Step 1: methyl 4-(1-(N-(tert-butoxycarbonyl)-O-(cyclohexylmethyl)-L-threonyl)piperidin-4-yl)benzoate: To a solution of methyl 4-(piperidin-4-yl)benzoate (274 mg, 1.25 mmol) in DCM (4 ml) was added N-(tert-butoxycarbonyl)-O-(cyclohexylmethyl)-L-threonine (427 mg, 1.35 mmol), HATU (468 mg, 1.23 mmol) and DIPEA (636 mg, 4.93 mmol) and the reaction stirred at room temperature for 2 h. The mixture was diluted with water (20 mL) and extracted with EtOAc (20 ml×5). The combined organic layers were washed with brine, dried over Na₂SO₄, filtered and concentrated. The residue obtained was purified by RP column (76% MeOH in H₂O) to afford methyl 4-(1-(N-(tert-butoxycarbonyl)-O-(cyclohexylmethyl)-L-threonyl)piperidin-4-yl)benzoate (470 mg, 74%) as colourless oil. LCMS m/z=539.2 [M+Na]⁺; ¹HNMR (400 MHz, DMSO-d₆) δ 7.90 (d, J=7.6 Hz, 2H), 7.38 (d, J=8.2 Hz, 2H), 4.50 (d, J=32.5 Hz, 2H), 4.18 (d, J=15.7 Hz, 1H), 3.83 (s, 3H), 3.59 (d, J=5.7 Hz, 1H), 3.27 (s, 1H), 3.14 (d, J=10.5 Hz, 2H), 2.91 (t, J=10.9 Hz, 1H), 2.68 (d, J=12.1 Hz, 1H), 1.90-1.77 (m, 2H), 1.72-1.58 (m, 6H), 1.47-1.40 (m, 2H), 1.38 (s, 9H), 1.23 (s, 1H), 1.14 (s, 2H), 1.05 (dd, J=14.6, 5.6 Hz, 3H), 0.87 (d, J=11.8 Hz, 2H).

Step 2: methyl 4-(1-(O-(cyclohexylmethyl)-L-threonyl)piperidin-4-yl)benzoate: To a solution of methyl 4-(1-(N-(tert-butoxycarbonyl)-O-(cyclohexylmethyl)-L-threonyl)piperidin-4-yl)benzoate (500 mg, 0.96 mmol) in DCM (5 mL) was added TFA (1.5 mL) and the reaction stirred at room temperature for 1 h. The mixture was concentrated to afford methyl 4-(1-(0-(cyclohexylmethyl)-L-threonyl)piperidin-4-yl)benzoate (400 mg, 100%) as white solid. LCMS m/z=417.3 [M+H]⁺.

### Synthesis of 3-allyl 9-(tert-butyl) 3,9-diazaspiro[5.5]undecane-3,9-dicarboxylate

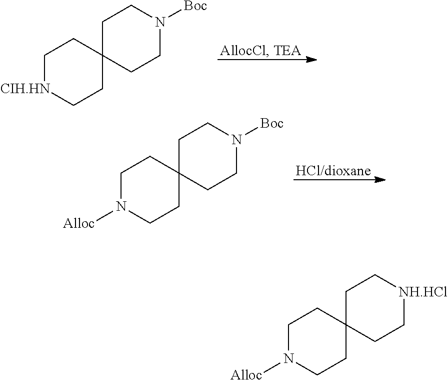

Step 1: 1-(2-(methoxycarbonyl)benzyl)-1H-pyrazole-4-carboxylic acid: To a solution of tert-butyl 1-(2-(methoxycarbonyl)benzyl)-1H-pyrazole-4-carboxylate (45 mg, 0.14 mmol) in DCM (2 mL) was added TFA (1 mL) and the reaction stirred at room temperature for 2 h. The solvent was removed under vacuum to afford 1-(2-(methoxycarbonyl)benzyl)-1H-pyrazole-4-carboxylic acid (37 mg, 100%) which was used directly in the next step. LCMS m/z=261.1 [M+H]⁺.

Step 2: 3-allyl 9-(tert-butyl) 3,9-diazaspiro[5.5]undecane-3,9-dicarboxylate: To a solution of tert-butyl 3,9-diazaspiro[5.5]undecane-3-carboxylate (800 mg, 2.75 mmol) in DCM (10 mL) at 0° C. was added TEA (835 mg, 8.25 mmol) and allyl chloroformate (332 mg, 2.75 mmol). The reaction was stirred at room temperature for 4 h then was diluted with water (30 mL), extracted with DCM (50 mL×3). The combined organic layers were washed with brine, dried over Na₂SO₄, filtered and concentrated to afford 3-allyl 9-(tert-butyl) 3,9-diazaspiro[5.5]undecane-3,9-dicarboxylate (930 mg, 100%) as a yellow oil. LCMS m/z=283.2 [M+H-56]⁺; ¹H NMR (400 MHz, DMSO-d₆) δ 5.92 (m, 1H), 5.30-5.22 (m, 1H), 5.17 (m, 1H), 4.50 (d, J=5.4 Hz, 2H), 3.40-3.34 (m, 4H), 3.29 (t, J=5.8 Hz, 4H), 1.38 (m, 17H).

### Synthesis of 1-(cyclohexylmethyl)-1H-pyrazole-4-carboxylic acid

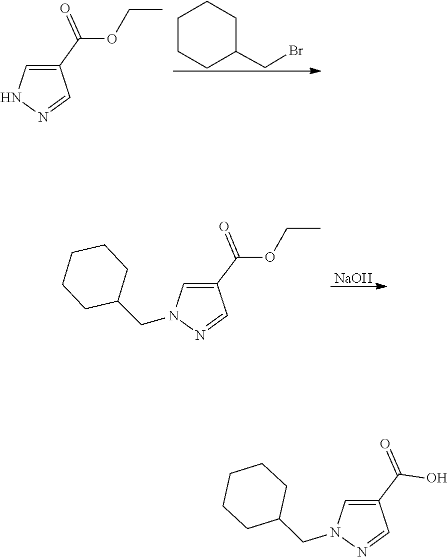

Step 1: ethyl 1-(cyclohexylmethyl)-1H-pyrazole-4-carboxylate: To a solution of ethyl 1H-pyrazole-4-carboxylate (1 g, 7.1 mmol) in MeCN (10 mL) was added (bromomethyl)cyclohexane (1.4 g, 7.8 mmol) and Cs₂CO₃ (4.6 g, 14.2 mmol). The reaction was stirred at room temperature overnight then was diluted with water (30 mL) and extracted with EtOAc (50 mL×3). The combined organic layers were washed with brine, dried over Na₂SO₄, filtered and concentrated to afford ethyl 1-(cyclohexylmethyl)-1H-pyrazole-4-carboxylate (1.5 g, 88%) as yellow oil. m/z=236.87 [M+H]⁺, ¹H NMR (400 MHz, CDCl3) δ 7.88 (s, 1H), 7.82 (s, 1H), 4.36-4.20 (m, 2H), 3.92 (d, J=7.2 Hz, 2H), 1.94-1.79 (m, 1H), 1.76-1.53 (m, 5H), 1.37-1.27 (m, 3H), 1.29-1.09 (m, 3H), 1.00-0.87 (m, 2H).

Step 2: 1-(cyclohexylmethyl)-1H-pyrazole-4-carboxylic acid: To a solution of ethyl 1-(cyclohexylmethyl)-1H-pyrazole-4-carboxylate (200 mg, 0.8 mmol) in a mixture of THF and water (2.5 mL/0.5 mL) was added NaOH (64 mg, 1.6 mmol). The mixture was stirred at 50° C. for 4 h. The reaction was diluted with water (15 mL) and extracted with EtOAc (15 mL). The aqueous layer was collected, acidified with 1M HCl to pH ~6 and extracted with EtOAc (30 mL×3). The combined organic layers were washed with brine, dried over Na₂SO₄, filtered and concentrated to afford 1-(cyclohexylmethyl)-1H-pyrazole-4-carboxylic acid (130 mg, 74%) as a white solid. m/z=208.78 [M+H]⁺; ¹H NMR (400 MHz, CD₃OD) δ 8.09 (s, 1H), 7.86 (s, 1H), 4.00 (d, J=7.4 Hz, 2H), 1.95-1.51 (m, 6H), 1.36-1.21 (m, 3H), 1.07-0.93 (m, 2H).

### Synthesis of 1-(4-(trifluoromethyl)benzyl)-1H-pyrazole-4-carboxylic acid

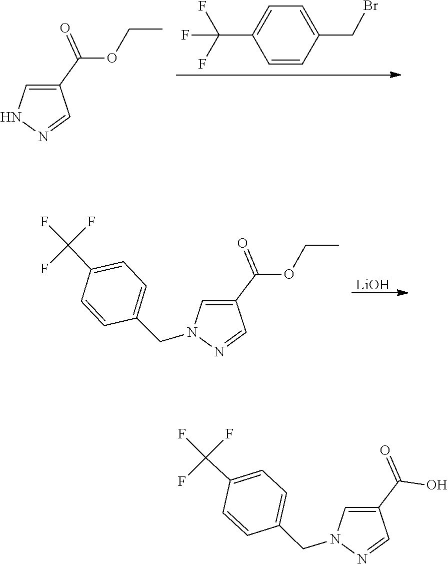

Step 1: methyl 1-(4-(trifluoromethyl)benzyl)-1H-pyrazole-4-carboxylate: To a solution of methyl 1H-pyrazole-4-carboxylate (500 mg, 3.57 mmol) and 1-(bromomethyl)-4-(trifluoromethyl)benzene (938 mg, 3.92 mmol) in DMF (5 mL) was added K₂CO₃ (986 mg, 7.14 mmol). The reaction mixture was stirred at room temperature under a N₂ atmosphere overnight then was diluted with water (30 mL) and extracted with EtOAc (30 mL×3). The combined organic layers were washed with brine, dried over Na₂SO₄, filtered and concentrated to afford methyl 1-(4-(trifluoromethyl)benzyl)-1H-pyrazole-4-carboxylate (967 mg, 96%) as a white solid. ¹H NMR (400 MHz, Chloroform-d) δ 7.93 (d, J=20.4 Hz, 2H), 7.62 (d, J=8.0 Hz, 2H), 7.33 (d, J=8.0 Hz, 2H), 5.37 (s, 2H), 4.35-4.22 (m, 2H), 1.34 (td, J=7.0, 1.2 Hz, 3H).

Step 2: 1-(4-(trifluoromethyl)benzyl)-1H-pyrazole-4-carboxylic acid: To a solution of methyl 1-(4-(trifluoromethyl)benzyl)-1H-pyrazole-4-carboxylate (967 mg, 3.24 mmol) in a mixture of THF (8 mL) and water (2 ml) was added LiOHH₂O (272 mg, 6.48 mmol). The reaction mixture was heated at 55° C. for 1 h then was diluted with water (20 mL) and extracted with EtOAc (30 mL×2). The aqueous layer was collected, acidified to pH-2 with 1M HCl and extracted with EtOAc (50 mL×2). The combined organic layers were washed with brine, dried over Na₂SO₄, filtered and concentrated to afford 1-(4-(trifluoromethyl)benzyl)-1H-pyrazole-4-carboxylic acid (812 mg, 93%) as a yellow solid. LCMS m/z=271.15 [M+H]⁺; ¹H NMR (400 MHz, Chloroform-d) δ 8.02 (s, 1H), 7.97 (s, 1H), 7.63 (d, J=8.0 Hz, 2H), 7.36 (d, J=8.0 Hz, 2H), 5.39 (s, 2H).

### Synthesis of methyl (R)-4-(2-((2-oxabicyclo[2.2.2]octan-4-yl)methoxy)-1-aminoethyl)benzoate

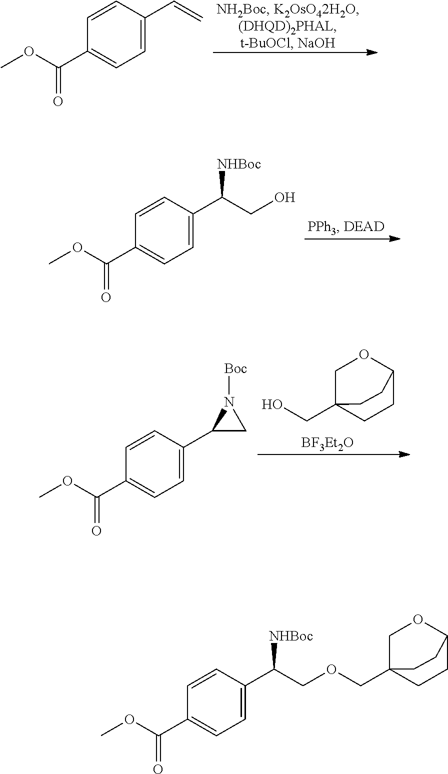

Step 1: methyl (R)-4-(1-((tert-butoxycarbonyl)amino)-2-hydroxyethyl)benzoate: NaOH (1.48 g, 37.0 mmol) was dissolved in water (100 mL) and 3 mL of this solution was used to dissolve K₂OsO₄·2H₂O (181 mg, 0.493 mmol) in a separate flask to give a purple solution. The remaining NaOH solution was cooled to 0° C. and was slowly added to a solution of tert-Butyl carbamate (5.06 g, 43.16 mmol) in n-Propanol (70 mL), tert-Butyl hypochlorite (4.02 g, 37.0 mmol) and the reaction stirred for 5 min. A solution of (DHQD)₂PHAL (576 mg, 0.74 mmol) in n-Propanol (40 mL) was added, followed by a solution of methyl 4-vinylbenzoate (2.0 g, 12.33 mmol) in n-Propanol (110 mL) and the purple solution of K₂OsO₄·2H₂O. The reaction was stirred at 0° C. for 1 h then was concentrated and extracted with EtOAc (200 mL×3). The combined organic layers were washed with brine, dried over Na₂SO₄, filtered and concentrated. The residue obtained was purified by prep-TLC (eluent: DCM:MeOH=30:1, v/v) to afford methyl (R)-4-(1-((tert-butoxycarbonyl)amino)-2-hydroxyethyl)benzoate (510 mg, 14%) as a white solid. LCMS m/z=318.1 [M+Na]⁻; ¹H NMR (400 MHz, DMSO-d₆) δ 7.91 (d, J=8.0 Hz, 2H), 7.43 (d, J=8.0 Hz, 2H), 7.31 (d, J=8.2 Hz, 1H), 4.85 (t, J=5.8 Hz, 1H), 4.59 (d, J=7.6 Hz, 1H), 3.85 (s, 3H), 3.50 (t, J=6.6 Hz, 2H), 1.37 (s, 9H).

Step 2: tert-butyl (R)-2-(4-(methoxycarbonyl)phenyl)aziridine-1-carboxylate: To a solution of methyl (R)-4-(1-((tert-butoxycarbonyl)amino)-2-hydroxyethyl)benzoate (250 mg, 0.85 mmol) in toluene (3 mL) at 0° C. under a N₂ atmosphere was added PPh₃ (267 mg, 1.02 mmol) and DEAD (177 mg, 1.02 mmol). The reaction was heated at 80° C. overnight then was diluted with water (20 mL) and extracted with EtOAc (80 mL×3). The combined organic layers were washed with brine, dried over Na₂SO₄, filtered and concentrated. The mixture was purified by prep-TLC (eluent: Pet. Ether:EtOAc=5:1, v/v) to afford tert-butyl (R)-2-(4-(methoxycarbonyl)phenyl)aziridine-1-carboxylate (170 mg, 72%) as a white solid. ¹H NMR (400 MHz, DMSO-d₆) δ 7.94 (d, J=8.4 Hz, 2H), 7.43 (d, J=8.4 Hz, 2H), 3.85 (s, 3H), 3.64-3.60 (m, 1H), 2.69 (d, J=6.4 Hz, 1H), 2.30 (d, J=3.5 Hz, 1H), 1.39 (s, 9H).

Step 3: methyl (R)-4-(2-((2-oxabicyclo[2.2.2]octan-4-yl)methoxy)-1-aminoethyl)benzoate: To a mixture of tert-butyl (R)-2-(4-(methoxycarbonyl)phenyl)aziridine-1-carboxylate (200 mg, 0.721 mmol) and (2-oxabicyclo[2.2.2]octan-4-yl)methanol (307 mg, 2.16 mmol) was added boron trifluoride etherate (205 mg, 1.44 mmol). The reaction mixture was stirred at room temperature for 2 h then was diluted with EtOAc (25 mL) and washed with brine. The combined organic layers were dried over Na₂SO₄, filtered and concentrated. The residue was purified by prep-TLC (eluent: DCM:MeOH=10:1) to afford methyl (R)-4-(2-((2-oxabicyclo[2.2.2]octan-4-yl)methoxy)-1-aminoethyl)benzoate (48 mg, 16%) as a colorless oil. LCMS m/z=320.2 [M+H]⁺; ¹H NMR (400 MHz, DMSO-d6) δ 7.96 (d, J=8.0 Hz, 2H), 7.43 (d, J=8.0 Hz, 2H), 4.33 (t, J=5.1 Hz, 1H), 3.85 (s, 3H), 3.66 (s, 1H), 3.62 (s, 2H), 2.99-2.87 (m, 2H), 2.81 (d, J=6.2 Hz, 2H), 1.91-1.80 (m, 2H), 1.60-1.41 (m, 6H).

### Synthesis of methyl (S)-4-(2-((2-oxabicyclo[2.2.2]octan-4-yl)methoxy)-1-aminoethyl)benzoate

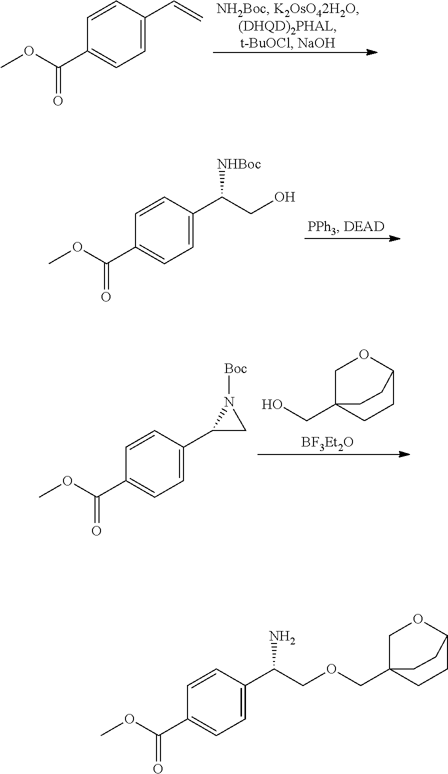

Step 1: methyl (S)-4-(1-((tert-butoxycarbonyl)amino)-2-hydroxyethyl)benzoate: NaOH (1.48 g, 37.0 mmol) was dissolved in water (100 mL) and 3 mL of this solution was used to dissolve K₂OsO₄·2H₂O (181 mg, 0.493 mmol) in a separate flask to give a purple solution. The remaining NaOH solution was cooled to 0° C. and was slowly added to a solution of tert-Butyl carbamate (5.06 g, 43.16 mmol) in n-Propanol (70 mL), tert-Butyl hypochlorite (4.02 g, 37.0 mmol) was added and the reaction stirred for 5 min. A Solution of (DHQ)₂PHAL (576 mg, 0.74 mmol) in n-Propanol (40 mL) was added, followed by a solution of methyl 4-vinylbenzoate (2.0 g, 12.33 mmol) in n-Propanol (110 mL) and the purple solution of K₂OsO₄·2H₂O. The reaction mixture was stirred at 0° C. for 1 h then was concentrated and extracted with EtOAc (200 mL×3). The combined organic layers were washed with brine, dried over Na₂SO₄, filtered and concentrated. The residue was purified by prep-TLC (eluent: DCM:MeOH=30:1, v/v) to afford methyl (S)-4-(1-((tert-butoxycarbonyl)amino)-2-hydroxyethyl)benzoate (510 mg, 14%) as a white solid. LCMS m/z=318.1 [M+Na]⁺; ¹H NMR (400 MHz, DMSO-d₆) δ 7.90 (d, J=8.0 Hz, 2H), 7.43 (d, J=8.0 Hz, 2H), 7.31 (d, J=8.2 Hz, 1H), 4.84 (t, J=5.8 Hz, 1H), 4.66-4.51 (m, 1H), 3.84 (s, 3H), 3.51 (t, J=6.6 Hz, 2H), 1.36 (s, 9H).

Step 2: tert-butyl (S)-2-(4-(methoxycarbonyl)phenyl)aziridine-1-carboxylate: To a solution of methyl (S)-4-(1-((tert-butoxycarbonyl)amino)-2-hydroxyethyl)benzoate (500 mg, 1.69 mmol) in toluene (10 mL) at 0° C. under a N₂ atmosphere was added PPh₃ (533 mg, 2.03 mmol) and DEAD (354 mg, 2.03 mmol). The reaction was heated at 80° C. overnight then was diluted with water (20 mL) and extracted with EtOAc (80 mL×3). The combined organic layers were washed with brine, dried over Na₂SO₄, filtered and concentrated. The mixture was purified by prep-TLC (eluent: Pet. Ether:EtOAc=5:1) to afford tert-butyl (S)-2-(4-(methoxycarbonyl)phenyl)aziridine-1-carboxylate (310 mg, 66%) as a white solid. ¹H NMR (400 MHz, DMSO-d₆) δ 7.93 (d, J=8.4 Hz, 2H), 7.43 (d, J=8.4 Hz, 2H), 3.85 (s, 3H), 3.63 (dd, J=6.4, 3.4 Hz, 1H), 2.69 (d, J=6.4 Hz, 1H), 2.30 (d, J=3.4 Hz, 1H), 1.39 (s, 9H).

Step 3: methyl (S)-4-(2-((2-oxabicyclo[2.2.2]octan-4-yl)methoxy)-1-aminoethyl)benzoate: To a mixture of tert-butyl (S)-2-(4-(methoxycarbonyl)phenyl)aziridine-1-carboxylate (200 mg, 0.721 mmol) in (2-oxabicyclo[2.2.2]octan-4-yl)methanol (307 mg, 2.16 mmol) was added boron trifluoride etherate (205 mg, 1.44 mmol). The reaction mixture was stirred at room temperature for 2 h then was diluted with EtOAc (25 mL) and washed with brine. The combined organic layers were dried over Na₂SO₄, filtered and concentrated. The residue was purified by prep-TLC (eluent: DCM:MeOH=10:1, v/v) to afford methyl (S)-4-(2-((2-oxabicyclo[2.2.2]octan-4-yl)methoxy)-1-aminoethyl)benzoate (70 mg, 30%) as a colorless oil LCMS m/z=320.1 [M+H]⁺; ¹H NMR (400 MHz, DMSO-d₆) δ 7.97 (d, J=8.0 Hz, 2H), 7.44 (d, J=8.0 Hz, 2H), 4.50-4.33 (m, 1H), 3.85 (s, 3H), 3.68-3.60 (m, 2H), 3.51-3.41 (m, 2H), 3.40-3.32 (m, 2H), 2.99-2.83 (m, 3H), 1.91-1.74 (m, 2H), 1.66-1.33 (m, 6H).

### Synthesis of methyl 4-(piperidin-4-yl)benzoate hydrochloride

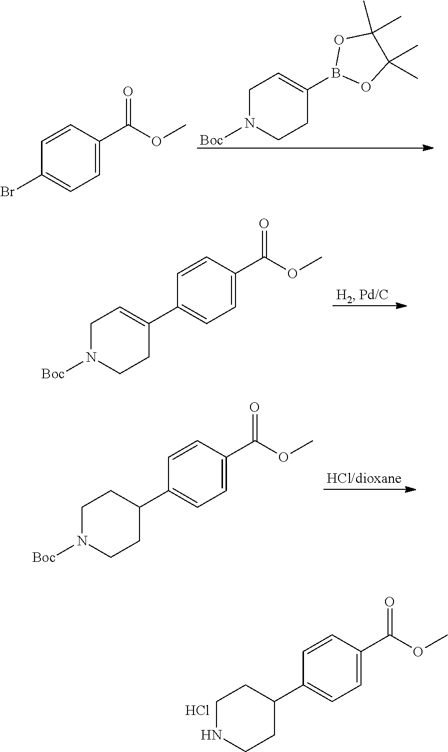

Step 1: tert-butyl 4-(4-(methoxycarbonyl)phenyl)-3,6-dihydropyridine-1 (2H)-carboxylate: To a solution of methyl 4-bromobenzoate (1.0 g, 4.7 mmol) in a mixture of dioxane (10 mL) and water (2 mL) was added tert-butyl 4-(4,4,5,5-tetramethyl-1,3,2-dioxaborolan-2-yl)-3,6-dihydropyridine-1 (2H)-carboxylate (1.75 g, 5.6 mmol), K₂CO₃ (1.9 g, 14.1 mmol) and Pd(dppf)Cl₂ (0.17 g, 0.235 mmol). The reaction was heated at 100° C. for 2 h under a N₂ atmosphere then was cooled to room temperature, diluted with water (50 mL) and extracted with EtOAc (20 mL×3). The combined organic layers were washed with brine, dried over Na₂SO₄ filtered and concentrated. The mixture was purified by column chromatography on silica gel (eluent: Pet. ether:EtOAc=60:1) to afford tert-butyl 4-(4-(methoxycarbonyl)phenyl)-3,6-dihydropyridine-1 (2H)-carboxylate (1.27 g, 86%) as a white solid. LCMS m/z=262.1 [M-55]⁺; ¹H NMR (400 MHz, Chloroform-d) δ 8.00 (d, J=8.4 Hz, 2H), 7.43 (d, J=8.4 Hz, 2H), 6.16 (s, 1H), 4.10 (s, 2H), 3.91 (s, 3H), 3.65 (t, J=5.5 Hz, 2H), 2.54 (s, 2H), 1.49 (s, 9H).

Step 2: tert-butyl 4-(4-(methoxycarbonyl)phenyl)piperidine-1-carboxylate: To a solution of tert-butyl 4-(4-(methoxycarbonyl)phenyl)-3,6-dihydropyridine-1 (2H)-carboxylate (1.27 g, 4.0 mmol) in MeOH (10 mL) was added 10% Pd/C (120 mg) and the reaction stirred under a H₂ atmosphere for 3 h. The catalyst was removed by filtration through Celite and the filtrate concentrated to afford tert-butyl 4-(4-(methoxycarbonyl)phenyl)piperidine-1-carboxylate (1.04 g, 81%) as a grey solid. LCMS m/z=263.8 [M-55]⁺; ¹H NMR (400 MHz, Chloroform-d) δ 7.98 (d, J=8.0 Hz, 2H), 7.28 (d, J=8.0 Hz, 2H), 4.26 (s, 2H), 3.90 (s, 3H), 2.81 (t, J=12.6 Hz, 2H), 2.75-2.65 (m, 1H), 1.83 (d, J=12.6 Hz, 2H), 1.65 (t, J=10.6 Hz, 2H), 1.48 (s, 9H).

Step 3: methyl 4-(piperidin-4-yl)benzoate hydrochloride: To a solution of tert-butyl 4-(4-(methoxycarbonyl)phenyl)piperidine-1-carboxylate (1.04 g, 3.3 mmol) in dioxane (10 mL) was added a solution of HCl in dioxane (4M, 4 mL, 16 mmol). The resulting mixture was stirred for 3 h, then the solvent was removed under reduced pressure to afford methyl 4-(piperidin-4-yl)benzoate hydrochloride (714 mg, quant.) as a white solid. LCMS m/z=220.0[M+H]⁺; ¹HNMR (400 MHz, Chloroform-d) δ 8.00 (d, J=7.9 Hz, 2H), 7.32 (d, J=8.0 Hz, 2H), 3.91 (s, 3H), 3.65 (d, J=10.4 Hz, 2H), 3.03 (s, 2H), 2.84 (t, J=10.4 Hz, 1H), 2.27 (q, J=12.0, 11.4 Hz, 2H), 2.06 (d, J=13.4 Hz, 2H), 1.74 (s, 1H).

### Synthesis of 5-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)picolinic acid (I-92)

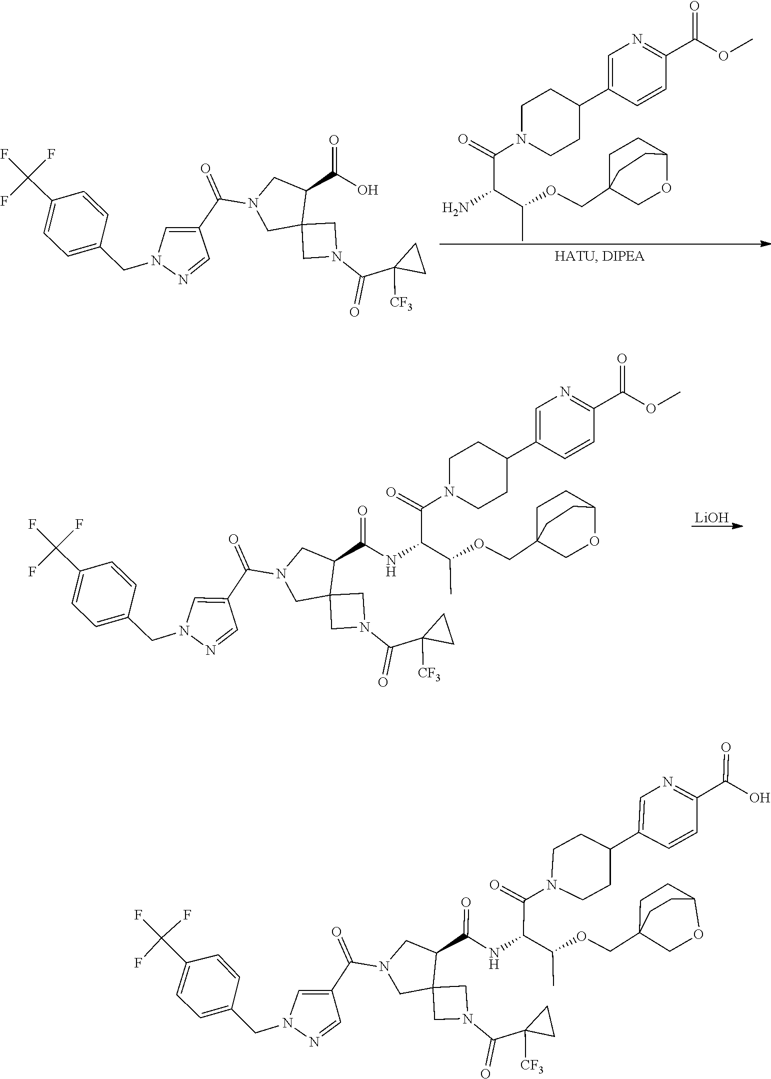

Step 1: methyl 5-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)picolinate To a solution of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (110 mg, 0.20 mmol) in DCM (2 mL) was added HATU (92 mg, 0.24 mmol) and DIPEA (78 mg, 0.61 mmol) and the mixture stirred at room temperature for 30 min. Methyl 5-(1-(O-((2-oxabicyclo[2.2.2]octan-4-yl)methyl)-L-threonyl)piperidin-4-yl)picolinate (90 mg, 0.20 mmol) was added and stirring continued for 2 h. The reaction was diluted with water (20 mL) and extracted with DCM (50 mL×3). The combined organic layers were washed with brine, dried over Na₂SO₄, filtered and concentrated. The residue obtained was purified by prep-TLC (eluent: DCM:MeOH=15:1) to afford methyl 5-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)picolinate (60 mg, 31%) as a white solid. LCMS m/z=972.3 [M+H]⁺; ¹H NMR (400 MHz, DMSO-d₆) δ 8.61 (s, 1H), 8.45-8.32 (m, 2H), 8.03-7.91 (m, 1H), 7.90-7.78 (m, 2H), 7.75-7.67 (m, 2H), 7.49-7.39 (m, 2H), 5.51-5.44 (m, 2H), 4.99-4.83 (m, 1H), 4.61-4.47 (m, 1H), 4.42-3.95 (m, 5H), 3.86 (s, 3H), 3.81-3.36 (m, 9H), 3.24-2.86 (m, 5H), 1.93-1.76 (m, 3H), 1.65-1.31 (m, 9H), 1.29-1.26 (m, 3H), 1.15-0.96 (m, 4H).

Step 2: 5-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)picolinic acid I-92 To a solution of methyl 5-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)picolinate (60 mg, 0.06 mmol) in a mixture of THF (1.6 mL), MeOH (0.4 mL) and H₂O (0.4 mL) was added LiOH (5 mg, 0.12 mmol). The reaction was stirred at room temperature for 2 h then was diluted with water (20 mL) and extracted with EtOAc (10 mL×2). The aqueous phase was collected, acidified with 1M HCl to pH ~4 and extracted with EtOAc (20 mL×3). The combined organic layers were washed with brine, dried over Na₂SO₄ filtered and concentrated. The residue obtained was purified by prep-HPLC to afford 5-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)picolinic acid (6.3 mg, 11%) as a white solid. LCMS m/z=958.3 [M+H]⁺; ¹H NMR (400 MHz, DMSO-d₆) δ 8.60 (s, 1H), 8.42 (s, 1H), 7.99 (s, 1H), 7.85 (d, J=17.3 Hz, 2H), 7.71 (d, J=7.9 Hz, 2H), 7.44 (d, J=7.9 Hz, 2H), 5.47 (d, J=4.1 Hz, 2H), 4.89 (s, 1H), 4.55 (s, 1H), 4.09 (d, J=75.6 Hz, 9H), 3.59 (d, J=25.6 Hz, 6H), 3.14 (s, 2H), 2.99 (s, 2H), 1.85 (d, J=31.3 Hz, 3H), 1.61-1.34 (m, 9H), 1.16 (s, 3H), 1.04 (s, 4H).

### Synthesis of 2-((4-((S)-8-(((2S,3R)-3-((2-oxabicyclo[2.2.2]octan-4-yl)methoxy)-1-oxo-1-(3,9-diazaspiro[5.5]undecan-3-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (I-60)

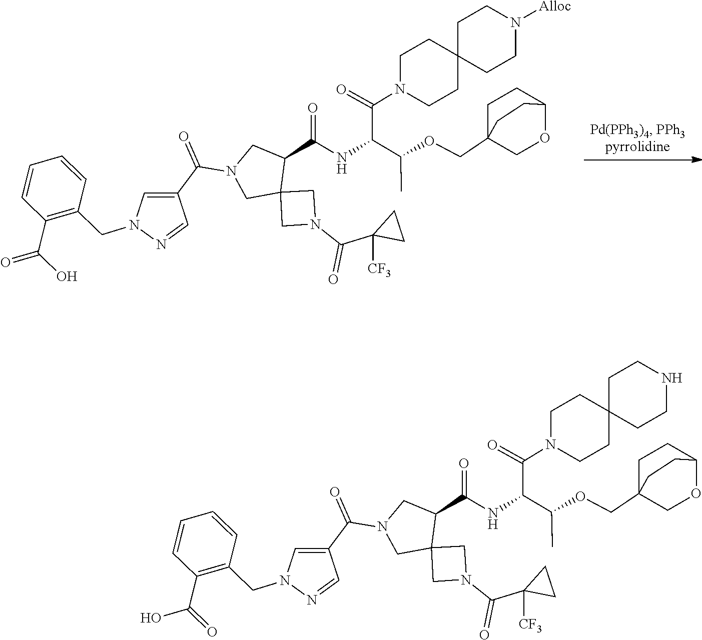

To a solution of 2-((4-((S)-8-(((2S,3R)-3-((2-oxabicyclo[2.2.2]octan-4-yl)methoxy)-1-(9-((allyloxy)carbonyl)-3,9-diazaspiro[5.5]undecan-3-yl)-1-oxobutan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (57 mg, 0.06 mmol) in DCM (3 mL) was added Pd(PPh₃)₄ (7 mg, 0.006 mmol), PPh₃ (4 mg, 0.01 mmol) and pyrrolidine (5 mg, 0.07 mmol). The reaction was stirred at room temperature for 2 h then the solvent was removed and the residue obtained purified by prep-HPLC to afford 2-((4-((S)-8-(((2S,3R)-3-((2-oxabicyclo[2.2.2]octan-4-yl)methoxy)-1-oxo-1-(3,9-diazaspiro[5.5]undecan-3-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (15 mg, 28%) as a white solid. LCMS m/z=882.4 [M+H]⁺; ¹H NMR (400 MHz, Chloroform-d) δ 8.66 (d, J=9.8 Hz, 1H), 8.25 (s, 1H), 8.12 (s, 1H), 7.79-7.70 (m, 2H), 7.38-7.28 (m, 2H), 5.92 (t, J=13.2 Hz, 1H), 5.55-5.38 (m, 1H), 4.58 (t, J=9.2 Hz, 1H), 4.27-3.99 (m, 4H), 3.95 (d, J=9.8 Hz, 2H), 3.80 (d, J=12.8 Hz, 2H), 3.71-3.49 (m, 10H), 3.08 (m, 2H), 2.93 (m, 2H), 2.73 (m, 1H), 1.88-1.66 (m, 3H), 1.47 (m, 8H), 1.37 (m, 3H), 1.25 (m, 2H), 1.14 (m, 4H), 0.99 (t, J=7.0 Hz, 3H).

### Synthesis of 4-(1-(O-((2-oxabicyclo[2.2.2]octan-4-yl)methyl)-N—((S)-6-(1-(4-cyanobenzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoic acid (I-93)

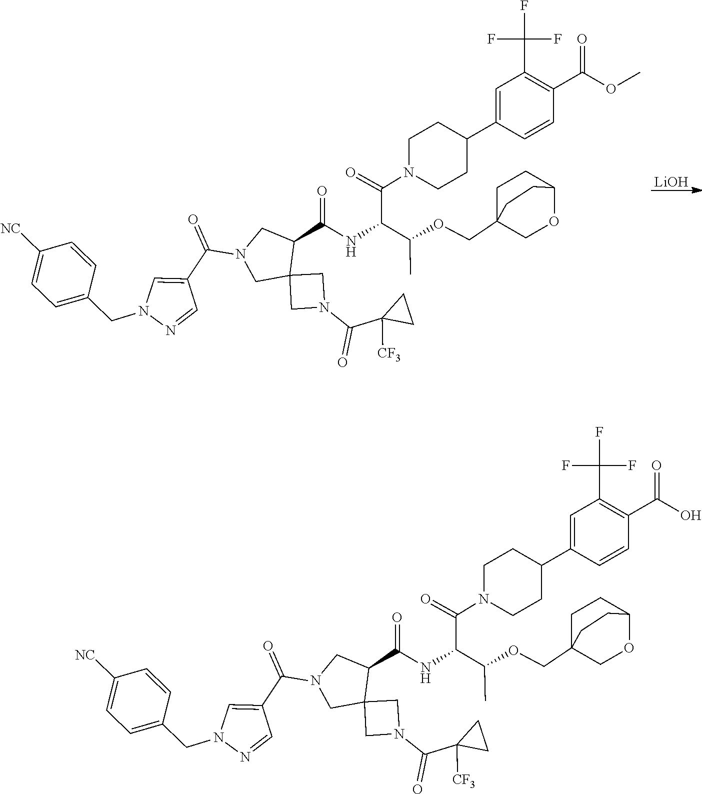 [SMILES: COC(=O)c1ccc(C2CCN(C(=O)[C@@H](NC(=O)[C@@H]3CN(C(=O)c4cnn(Cc5ccc(C#N)cc5)c4)CC34CN(C(=O)C3(C(F)(F)F)CC3)C4)[C@@H](C)OCC34CCC(CC3)OC4)CC2)cc1C(F)(F)F ; "LiOH" ; C[C@@H](OCC12CCC(CC1)OC2)[C@H](NC(=O)[C@@H]1CN(C(=O)c2cnn(Cc3ccc(C#N)cc3)c2)CC12CN(C(=O)C1(C(F)(F)F)CC1)C2)C(=O)N1CCC(c2ccc(C(=O)O)c(C(F)(F)F)c2)CC1]

To a solution of methyl 4-(1-(O-((2-oxabicyclo[2.2.2]octan-4-yl)methyl)-N—((S)-6-(1-(4-cyanobenzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoate (50 mg, 0.05 mmol) in a mixture of THF, water and MeOH (1.5 mL/0.5 mL/0.5 mL) was added LiOH (6.3 mg, 0.15 mmol) nd the mixture stirred at room temperature for 3 h. The reaction was diluted with water (20 mL) and extracted with EtOAc (20 mL). The aqueous layer was collected, acidified to pH ~2 with 1M HCl and extracted with EtOAc (20 mL×2). The combined organic layers were washed with brine, dried over Na₂SO₄, filtered and concentrated. The residue obtained was purified by prep-HPLC to afford 4-(1-(O-((2-oxabicyclo[2.2.2]octan-4-yl)methyl)-N—((S)-6-(1-(4-cyanobenzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoic acid (40 mg, 43%) as a white solid. LCMS m/z=982.4 [M+H]⁺; ¹H NMR (400 MHz, Chloroform-d) δ 7.99-7.83 (m, 3H), 7.67-7.55 (m, 3H), 7.37 (m, 4H), 5.38 (s, 2H), 5.05 (s, 1H), 4.81 (s, 1H), 4.05 (m, 10H), 3.79 (s, 1H), 3.69 (s, 1H), 3.60 (s, 1H), 3.20 (m, 3H), 2.96 (m, 2H), 2.75 (s, 1H), 2.01 (s, 3H), 1.70 (s, 8H), 1.44 (s, 2H), 1.22 (d, J=3.4 Hz, 3H), 1.17-1.06 (m, 3H).

Table I-15: The compound listed in Table I-15 were synthesized according to the procedures outlined for I-93 using the appropriate commercially available reagents and/or intermediates described elsewhere.

TABLE I-15

| Ex- | | | |
|---|---|---|---|
| am- | | | |
| ple | | | |
| Num- | | | |
| ber | Compound | ¹H NMR | LCMS |
| I-94 | 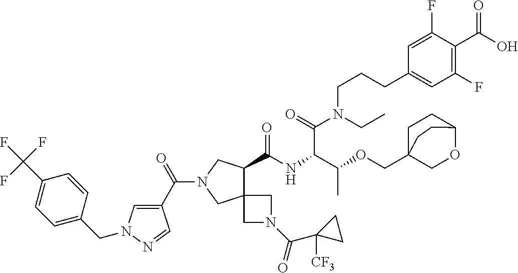 [SMILES: CCN(CCCc1cc(F)c(C(=O)O)c(F)c1)C(=O)[C@@H](NC(=O)[C@@H]1CN(C(=O)c2cnn(Cc3ccc(C(F)(F)F)cc3)c2)CC12CN(C(=O)C1(C(F)(F)F)CC1)C2)[C@@H](C)OCC12CCC(CC1)OC2] | ¹H NMR (400 MHz, DMSO-d₆) δ 8.36 (d, J = 28.4 Hz, 2H), 7.85 (d, J = 14.2 Hz, 1H), 7.72 (d, J = 8.0 Hz, 2H), 7.44 (d, J = 7.8 Hz, 2H), 7.07 (s, 2H), 5.47 (s, 2H), 4.99-3.69 (m, 9H), 3.59 (d, J = 26.4 Hz, 6H), 3.21-2.70 (m, 5H), 1.82 (s, 3H), 1.46 (d, J = 58.2 Hz, 8H), 1.29-0.97 (m, 8H). | m/z = 993.3 [M + H]⁺ |
| I-75 | 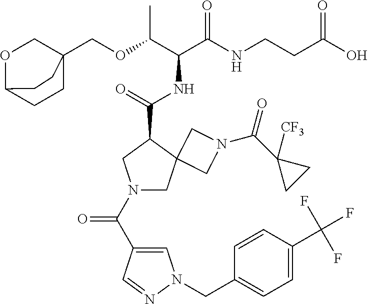 [SMILES: C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)NCCC(=O)O] | ¹H NMR (400 MHz, DMSO-d₆) δ 8.42- 7.83 (m, 4H), 7.72 (d, J = 8 Hz, 2H), 7.44 (d, J = 7.6 Hz, 2H), 5.52-5.48 (m, 2H), 4.32-3.72 (m, 10H), 3.63-3.43 (m, 6H), 3.12-2.92 (m, 4H), 1.81-1.37 (m, 8H), 1.15-0.94 (m, 7H). | m/z = 841.1 [M + H]⁺ |

### Synthesis of 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)-N-(methylsulfonyl)acetamide (1-95)

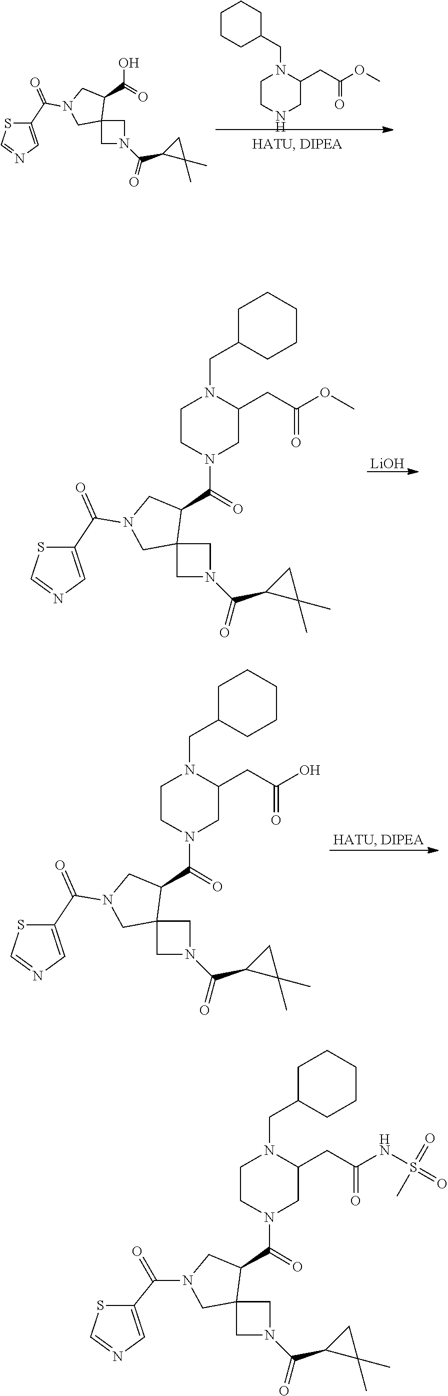

Step 1: methyl 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetate To a solution of (S)-2-((S)-2,2-dimethylcyclopropane-1-carbonyl)-6-(thiazole-5-carbonyl)-2,6-diazaspiro[3.4]octane-8-carboxylic acid (29 mg, 0.08 mmol) in DCM (2 mL) was added HATU (45 mg, 0.12 mmol) and DIPEA (41.28 mg, 0.32 mmol) and the mixture stirred at room temperature for 30 min. Methyl 2-(1-(cyclohexylmethyl)piperazin-2-yl)acetate (20 mg, 0.08 mmol) was added and stirring continued for 2 h. The solvent was removed under reduced pressure and the residue purified by prep-HPLC to afford methyl 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetate (14 mg, 29%) as a white solid. LCMS m/z=600.2[M+H]⁺; ¹H NMR (400 MHz, DMSO-d6) δ 9.25 (s, 1H), 8.39 (d, J=5.2 Hz, 1H), 4.11 (dd, J=29.4, 9.2 Hz, 4H), 3.81 (s, 5H), 3.62 (t, J=6.0 Hz, 6H), 3.30 (s, 1H), 2.89 (d, J=16.4 Hz, 2H), 2.58 (s, 1H), 2.40-2.22 (m, 3H), 2.06 (s, 1H), 1.63 (s, 5H), 1.40 (s, 2H), 1.10 (d, J=8.6 Hz, 6H), 1.05 (q, J=5.6, 5.2 Hz, 3H), 0.86 (s, 3H), 0.70-0.64 (m, 1H).

Step 2: 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetic acid To a solution of methyl 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetate (250 mg, 0.42 mmol) in a mixture of THF (2 mL), MeOH (0.5 mL) and water (0.5 mL) was added LiOH (30 mg, 1.26 mmol) and the reaction stirred at room temperature for 2 h. The mixture was diluted with water (50 mL) and extracted with EtOAc (30 mL×2). The aqueous phase was collected, acidified with 1M HCl to pH~2 and extracted with EtOAc (50 mL×3). The combined organic layers were washed with brine, dried over Na₂SO₄ filtered and concentrated. The residue obtained was purified by prep-HPLC to afford 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetic acid (40 mg, 16%) as a white solid. LCMS m/z=586.2 [M+H]⁺; ¹H NMR (400 MHz, DMSO-d6) δ 9.25 (s, 1H), 8.45-8.30 (m, 1H), 4.33-3.99 (m, 4H), 3.91-3.76 (m, 4H), 3.77-3.59 (m, 3H), 3.58-3.39 (m, 3H), 3.29 (s, 2H), 2.40-2.34 (m, 1H), 2.13-2.04 (m, 1H), 1.79-1.71 (m, 1H), 1.68-1.57 (m, 4H), 1.40 (s, 2H), 1.16 (s, 3H), 1.11 (s, 4H), 1.08-1.01 (m, 4H), 0.89-0.79 (m, 3H), 0.67 (s, 1H).

Step 3: 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)-N-(methylsulfonyl)acetamide I-95 To a solution of 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)acetic acid (20 mg, 0.034 mmol) in DCM (2 mL) was added HATU (13 mg, 0.034 mmol) and DIPEA (13 mg, 0.102 mmol) and the mixture stirred at room temperature for 30 min. Methanesulfonamide (4 mg, 0.034 mmol) was added and stirring continued for 2 h. The solvent was removed under reduced pressure and the residue obtained purified by prep-HPLC to afford 2-(1-(cyclohexylmethyl)-4-((S)-2-((S)-2,2-dimethylcyclopropane-1-carbonyl)-6-(thiazole-5-carbonyl)-2,6-diazaspiro[3.4]octane-8-carbonyl)piperazin-2-yl)-N-(methylsulfonyl)acetamide (8 mg, 8%) as a white solid as the first eluting diastereomer. LCMS m/z=663.2 [M+H]⁺; ¹H NMR (400 MHz, DMSO-d6) δ 9.25 (s, 1H), 8.39-8.29 (m, 1H), 5.94-5.83 (m, 1H), 4.34-3.98 (m, 3H), 3.95-3.54 (m, 8H), 3.13 (s, 10H), 2.13 (s, 2H), 1.75-1.60 (m, 4H), 1.47-1.02 (m, 12H), 0.89-0.76 (m, 3H), 0.67 (s, 1H).

### Synthesis of 4-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoic acid (I-89)

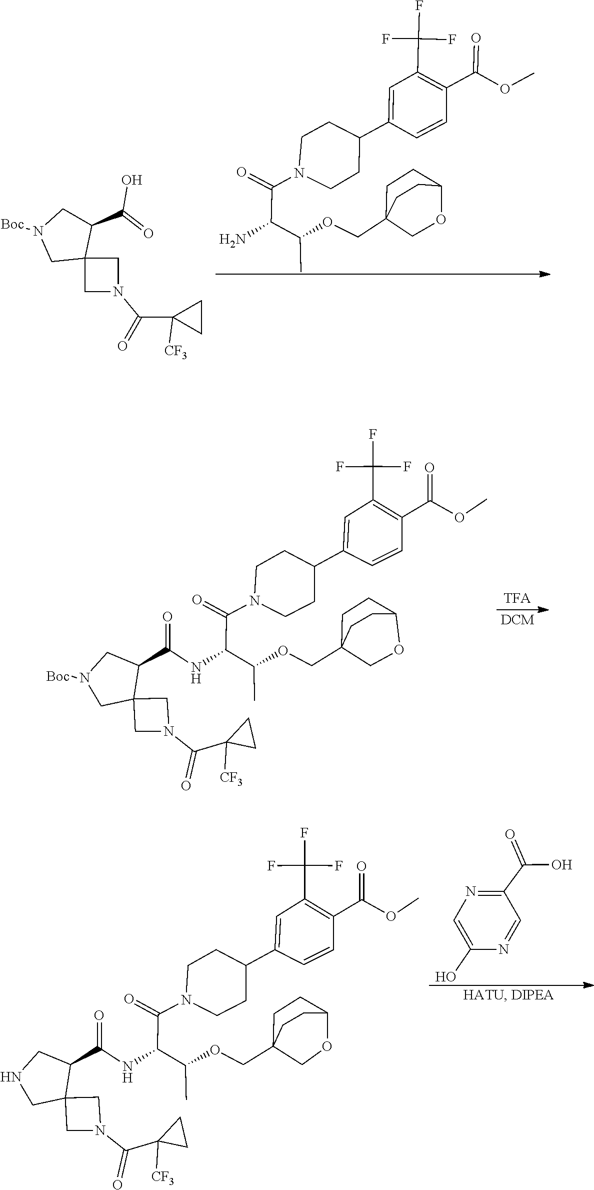

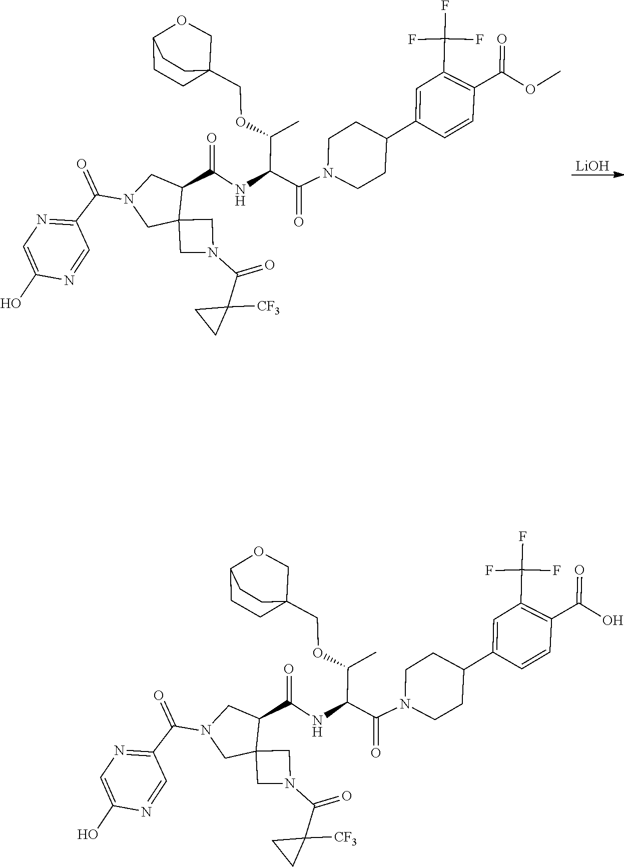 [SMILES: COC(=O)c1ccc(C2CCN(C(=O)[C@@H](NC(=O)[C@@H]3CN(C(=O)c4cnc(O)cn4)CC34CN(C(=O)C3(C(F)(F)F)CC3)C4)[C@@H](C)OCC34CCC(CC3)OC4)CC2)cc1C(F)(F)F ; "LiOH" ; C[C@@H](OCC12CCC(CC1)OC2)[C@H](NC(=O)[C@@H]1CN(C(=O)c2cnc(O)cn2)CC12CN(C(=O)C1(C(F)(F)F)CC1)C2)C(=O)N1CCC(c2ccc(C(=O)O)c(C(F)(F)F)c2)CC1]

Step 1: tert-butyl (S)-8-(((2S,3R)-3-((2-oxabicyclo[2.2.2]octan-4-yl)methoxy)-1-(4-(4-(methoxycarbonyl)-3-(trifluoromethyl)phenyl)piperidin-1-yl)-1-oxobutan-2-yl)carbamoyl)-2-(1-(trifluoromethyl)cyclopropane-1-carbonyl)-2,6-diazaspiro[3.4]octane-6-carboxylate To a solution of methyl 4-(1-(O-((2-oxabicyclo[2.2.2]octan-4-yl)methyl)-L-threonyl)piperidin-4-yl)-2-(trifluoromethyl)benzoate (120 mg, 0.30 mmol) in DCM (2 mL) was added HATU (116 mg, 0.31 mmol) and the mixture was stirred at room temperature for 30 mins. (S)-6-(tert-butoxycarbonyl)-2-(1-(trifluoromethyl)cyclopropane-1-carbonyl)-2,6-diazaspiro[3.4]octane-8-carboxylic acid (172 mg, 0.33 mmol) and DIPEA (118 mg, 0.91 mmol) were added and stirring continued for 2 h. The mixture was diluted with water (20 mL) and extracted with DCM (50 mL×3). The combined organic layers were washed with brine, dried over Na₂SO₄, filtered and concentrated. The residue was purified by RP-column (H₂O/MeCN=30/70, V/V) to afford (S)-8-(((2S,3R)-3-((2-oxabicyclo[2.2.2]octan-4-yl)methoxy)-1-(4-(4-(methoxycarbonyl)-3-(trifluoromethyl)phenyl)piperidin-1-yl)-1-oxobutan-2-yl)carbamoyl)-2-(1-(trifluoromethyl)cyclopropane-1-carbonyl)-2,6-diazaspiro[3.4]octane-6-carboxylate (340 mg, 100%) as a white solid. LCMS m/z=887.3[M+H]⁺; ¹H NMR (400 MHz, DMSO-d6) δ 8.29 (s, 1H), 7.84-7.75 (m, 1H), 7.74-7.63 (m, 2H), 4.91 (s, 1H), 4.56 (s, 1H), 4.40-4.04 (m, 4H), 3.85 (s, 3H), 3.66-3.35 (m, 10H), 3.28-2.96 (m, 5H), 2.08-1.65 (m, 6H), 1.61-1.50 (m, 5H), 1.41-1.34 (m, 11H), 1.15 (s, 3H), 1.09-0.97 (m, 3H).

Step 2: methyl 4-(1-(O-((2-oxabicyclo[2.2.2]octan-4-yl)methyl)-N—((S)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoate To a solution of tert-butyl (S)-8-(((2S,3R)-3-((2-oxabicyclo[2.2.2]octan-4-yl)methoxy)-1-(4-(4-(methoxycarbonyl)-3-(trifluoromethyl)phenyl)piperidin-1-yl)-1-oxobutan-2-yl)carbamoyl)-2-(1-(trifluoromethyl)cyclopropane-1-carbonyl)-2,6-diazaspiro[3.4]octane-6-carboxylate (100 mg, 0.1 mmol) in DCM (1.2 mL) was added TFA (0.3 mL) and the reaction at room temperature for 2 h. The solvent was removed under reduced pressure to afford methyl 4-(1-(O-((2-oxabicyclo[2.2.2]octan-4-yl)methyl)-N—((S)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoate (88 mg, 100%) which was used directly in the next step. LCMS m/z=787.2 [M+H]⁺.

Step 3: methyl 4-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoate To a solution of 5-hydroxypyrazine-2-carboxylic acid (40 mg, 0.28 mmol) in DCM (3 mL) was added HATU (109 mg, 0.28 mmol) and the mixture stirred at room temperature for 30 min. Methyl 4-(1-(O-((2-oxabicyclo[2.2.2]octan-4-yl)methyl)-N—((S)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoate (225 mg, 0.28 mmol) and DIPEA (258 mg, 2.0 mmol) were added and stirring continued for 2 h. The mixture was diluted with water (30 mL) and extracted with DCM (50 mL×3). The combined organic layers were washed with brine, dried over Na₂SO₄, filtered and concentrated. The residue was purified by RP-column (H₂O/MeCN=40/60, V/V) to afford methyl 4-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoate (170 mg, 65%) as a white solid. LCMS m/z=909.1 [M+H]⁺.

Step 4: 4-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoic acid I-89 To a solution of methyl 4-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoate (140 mg, 0.15 mmol) in a mixture of THF, water and EtOH (1.2 mL/0.3 mL/0.3 mL) was added LiOH (20 mg, 0.46 mmol) and the reaction stirred at room temperature for 4 h. The reaction was diluted with 1M HCl to a final pH of 2 and then concentrated. The residue obtained was purified by Prep-HPLC to afford 4-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoic acid (21.5 mg, 15%) as a white solid. LCMS m/z=895.2 [M+H]⁺; ¹H NMR (400 MHz, Methanol-d₄) δ 8.32-8.07 (m, 1H), 8.06-7.96 (m, 2H), 7.83-7.73 (m, 1H), 7.66 (s, 1H), 7.60-7.54 (m, 1H), 4.97 (s, 1H), 4.73-3.64 (m, 15H), 3.42-3.33 (m, 2H), 3.29-2.97 (m, 2H), 2.79 (s, 1H), 2.09-1.40 (m, 13H), 1.27-1.08 (m, 6H).

Synthesis of 4-(1-(O-((2-oxabicyclo[2.2.2]octan-4-yl)methyl)-N-(6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoic acid (I-89) To a solution of methyl 4-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoate (70 mg, 0.07 mmol) in a mixture of THF, H₂O and EtOH (0.8 mL/0.2 mL/0.2 mL) was added LiOH (10 mg, 0.23 mmol) and the reaction stirred at room temperature for 4 h. The reaction was diluted with 1M HCl to a final pH of 2 then was concentrated. The residue obtained was purified by Prep-HPLC to afford 4-(1-(O-((2-oxabicyclo[2.2.2]octan-4-yl)methyl)-N-(6-(5-hydroxypyrazine-2-carbonyl)-2-(1-(trifluoromethyl)cyclopropane-1-carbonyl)-2,6-diazaspiro[3.4]octane-8-carbonyl)-L-threonyl)piperidin-4-yl)-2-(trifluoromethyl)benzoic acid I-89 (A) (5 mg, 7%) as a white solid. LCMS m/z=895.2 [M+H]⁺; ¹H NMR (400 MHz, Methanol-d₄) δ 8.32-8.07 (m, 1H), 8.06-7.96 (m, 2H), 7.83-7.73 (m, 1H), 7.66 (s, 1H), 7.60-7.54 (m, 1H), 4.97 (s, 1H), 4.73-3.64 (m, 15H), 3.42-3.33 (m, 2H), 3.29-2.97 (m, 2H), 2.79 (s, 1H), 2.09-1.40 (m, 13H), 1.27-1.08 (m, 6H). Further elution provided a second diastereomer I-89 (B) (5.9 mg, 8%) as a white solid. LCMS m/z=895.3 [M+H]⁺; ¹H NMR (400 MHz, Methanol-d₄) δ 8.22-8.07 (m, 1H), 8.06-7.96 (m, 2H), 7.81-7.71 (m, 1H), 7.64 (s, 1H), 7.60-7.54 (m, 1H), 4.99 (s, 1H), 4.77-3.64 (m, 15H), 3.44-3.34 (m, 2H), 3.24-2.97 (m, 2H), 2.79 (s, 1H), 2.02-1.47 (m, 12H), 1.29-1.15 (m, 7H).

### Synthesis of (S)—N-((2S,3R)-3-((2-oxabicyclo[2.2.2]octan-4-yl)methoxy)-1-((4-fluorophenyl)sulfonamido)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide I-85

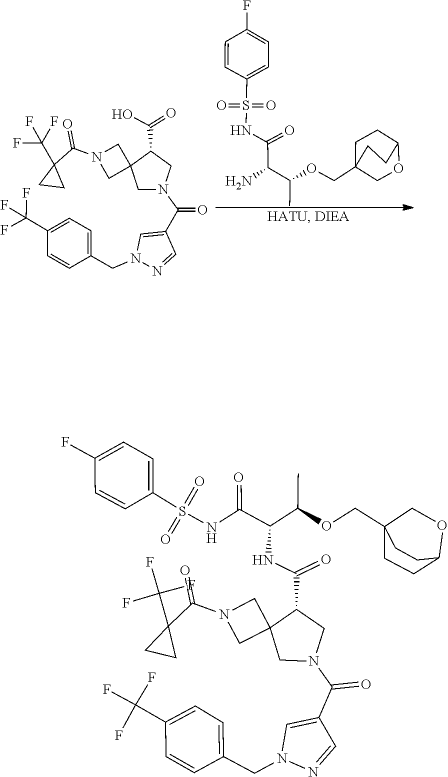 [SMILES: O=C(O)[C@@H]1CN(C(=O)c2cnn(Cc3ccc(C(F)(F)F)cc3)c2)CC12CN(C(=O)C1(C(F)(F)F)CC1)C2 ; C[C@@H](OCC12CCC(CC1)OC2)[C@H](N)C(=O)NS(=O)(=O)c1ccc(F)cc1 ; C[C@@H](OCC12CCC(CC1)OC2)[C@H](NC(=O)[C@@H]1CN(C(=O)c2cnn(Cc3ccc(C(F)(F)F)cc3)c2)CC12CN(C(=O)C1(C(F)(F)F)CC1)C2)C(=O)NS(=O)(=O)c1ccc(F)cc1]

To a solution of (2S,3R)-3-((2-oxabicyclo[2.2.2]octan-4-yl)methoxy)-2-amino-N-((4-fluorophenyl)sulfonyl)butanamide (125 mg, 0.31 mmol) in DCM (3 mL) was added HATU (119 mg, 0.31 mmol) and DIPEA (121 mg, 0.94 mmol) and the mixture stirred at room temperature for min. (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (170 mg, 0.31 mmol) was added and stirring continued for 2 h. The mixture was diluted with water (20 mL) and extracted with EtOAc (30 mL×3). The combined organic layers were washed with brine, dried over Na₂SO₄, filtered and concentrated. The residue obtained was purified by Prep-HPLC to afford (S)—N-((2S,3R)-3-((2-oxabicyclo[2.2.2]octan-4-yl)methoxy)-1-((4-fluorophenyl)sulfonamido)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (57 mg, 20%) as a white solid. LCMS m/z=927.1 [M+H]⁺; ¹H NMR (400 MHz, DMSO-d6) δ 8.41 (s, 1H), 8.19 (s, 1H), 8.05-7.96 (m, 2H), 7.85 (d, J=15.4 Hz, 1H), 7.72 (d, J=8.0 Hz, 2H), 7.45 (t, J=8.6 Hz, 4H), 5.48 (s, 2H), 4.46-3.67 (m, 10H), 3.60 (s, 2H), 3.40 (s, 2H), 2.96 (d, J=9.0 Hz, 1H), 2.61 (s, 1H), 1.76 (s, 2H), 1.47 (d, J=11.8 Hz, 2H), 1.35 (d, J=12.0 Hz, 2H), 1.25-1.12 (m, 6H), 0.92 (t, J=6.2 Hz, 3H).

Table I-16: The compound listed in Table I-16 were synthesized according to the procedures outlined for I-11 using the appropriate commercially available reagents and/or intermediates described elsewhere.

TABLE I-16

| Example | | | |
|---|---|---|---|
| Number | Compound | ¹H NMR | LCMS |
| I-84 | 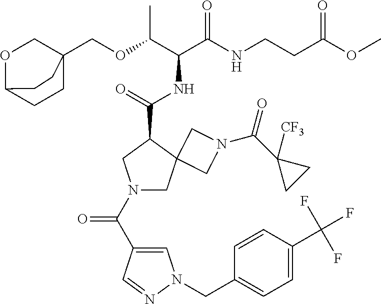 [SMILES: COC(=O)CCNC(=O)[C@@H](NC(=O)[C@@H]1CN(C(=O)c2cnn(Cc3ccc(C(F)(F)F)cc3)c2)CC12CN(C(=O)C1(C(F)(F)F)CC1)C2)[C@@H](C)OCC12CCC(CC1)OC2] | ¹H NMR (400 MHz, DMSO-d₆) δ 8.41 (d, J = 7.6 Hz, 1H), 8.18- 8.12 (m, 1H), 7.91- 7.84 (m, 2H), 7.72 (d, J = 8 Hz, 2H), 7.44 (d, J = 8 Hz, 2H), 5.48 (s, 2H), 4.33- 4.30 (m, 1H), 4.21- 4.16 (m, 1H), 4.01- 3.75 (m, 4H), 3.67- 3.35 (m, 11H), 3.27- 2.91 (m, 5H), 2.47- 2.45 (m, 1H), 1.82- 1.51 (m, 6H), 1.40- 1.30 (m, 2H), 1.20- 0.95 (m, 7H). | m/z = 855.2 [M + H]⁺ |

### Synthesis of 2-((cyclohexylmethyl)((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)amino)-N-(methylsulfonyl)acetamide I-72

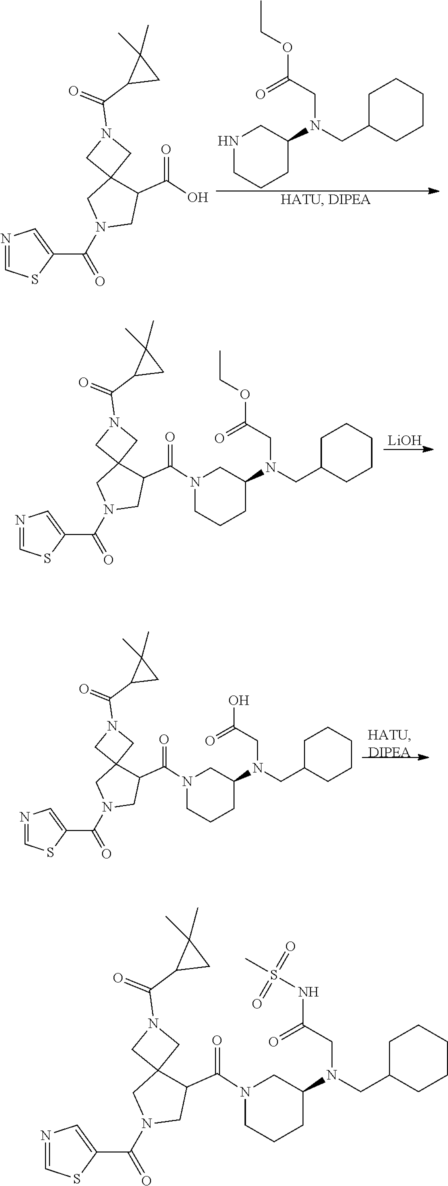

Step 1: ethyl N-(cyclohexylmethyl)-N—((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycinate To a solution of ethyl (S)-2-((S)-2,2-dimethylcyclopropane-1-carbonyl)-6-(thiazole-5-carbonyl)-2,6-diazaspiro[3.4]octane-8-carboxylic acid (188 mg, 0.52 mmol) in DCM (3 mL) was added HATU (197 mg, 0.52 mmol) and DIPEA (201 mg, 0.52 mmol) and the mixture stirred at room temperature for 30 min. (S)—N-(cyclohexylmethyl)-N-(piperidin-3-yl)glycinate (147 mg, 0.52 mmol) was added and stirring continued for 2 h. The solvent was removed under vacuum and the residue purified by prep-HPLC to afford ethyl N-(cyclohexylmethyl)-N—((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycinate (25 mg, 8%) as a white solid. LCMS m/z=628.4 [M+H]⁺; ¹H NMR (400 MHz, DMSO-d6) δ 9.26 (s, 1H), 8.44-8.32 (m, 1H), 4.60-4.01 (m, 8H), 3.81 (dd, J=21.8, 12.4 Hz, 8H), 3.05 (d, J=13.8 Hz, 1H), 1.74 (s, 4H), 1.63 (s, 4H), 1.55-1.27 (m, 4H), 1.24-1.02 (m, 15H), 0.86 (d, J=5.0 Hz, 2H), 0.70-0.65 (m, 1H).

Step 2: N-(cyclohexylmethyl)-N—((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycine To a solution of ethyl N-(cyclohexylmethyl)-N—((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycinate (200 mg, 0.32 mmol) in a mixture of THF (2 mL), MeOH (1 mL) and water (1 mL) was added LiOH (23 mg, 0.96 mmol). The mixture was stirred at room temperature for 2 h then was diluted with water (50 mL) and extracted with EtOAc (30 mL×2). The aqueous phase was collected, acidified with 1M HCl to pH ~2 and extracted with EtOAc (50 mL×3). The combined organic layers were washed with brine, dried over Na₂SO₄ filtered and concentrated to afford N-(cyclohexylmethyl)-N—((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycine (30 mg, 16%) as a yellow oil. LCMS m/z=600.3 [M+H]⁺.

Step 3: 2-((cyclohexylmethyl)((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)amino)-N-(methylsulfonyl)acetamide I-72 To a solution of N-(cyclohexylmethyl)-N—((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycine (30 mg, 0.5 mmol) in DCM (1 mL) was added HATU (28.5 mg, 0.75 mmol) and DIPEA (25.8 mg, 0.2 mmol) and he mixture stirred at room temperature for 30 min. Methanesulfonamide (4.75 mg, 0.05 mmol) was added and stirring continued for 2 h. The solvent was removed under reduced pressure and the residue obtained purified by prep-HPLC to afford 2-((cyclohexylmethyl)((S)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)amino)-N-(methylsulfonyl)acetamide (10 mg, 30%) as a white solid. LCMS m/z=677.4 [M+H]⁺; ¹H NMR (400 MHz, DMSO-d6) δ 9.25 (s, 1H), 8.39 (d, J=5.0 Hz, 1H), 4.43-3.62 (m, 16H), 3.21 (s, 1H), 3.16 (s, 3H), 1.68 (d, J=40.4 Hz, 8H), 1.37 (s, 3H), 1.11 (s, 7H), 1.04 (s, 3H), 0.85 (s, 2H), 0.68 (s, 1H).

### Synthesis of (S)—N-((2S,3R)-3-((2-oxabicyclo[2.2.2]octan-4-yl)methoxy)-1-((3-(methylsulfonamido)-3-oxopropyl)amino)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (I-73)

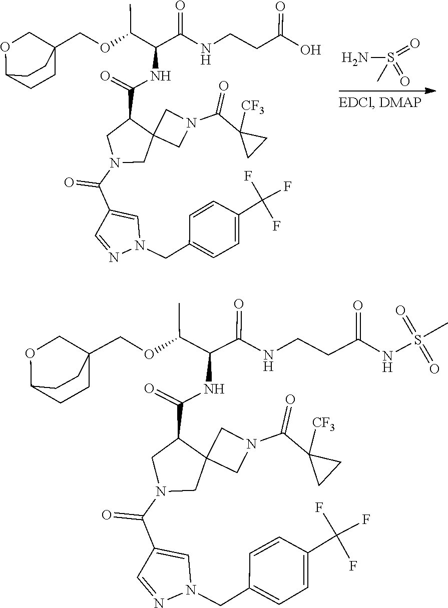

To a solution of 3-((2S,3R)-3-((2-oxabicyclo[2.2.2]octan-4-yl)methoxy)-2-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butanamido)propanoic acid (20 mg, 0.024 mmol) in DCE (1 mL) was added methanesulfonamide (2 mg, 0.017 mmol), EDCI (4 mg, 0.020 mmol) and DMAP (0.5 mg, 0.0036 mmol). The reaction was stirred at room temperature for 2 h then was diluted with water (10 mL), extracted with DCM (20 mL×3) and the combined organic layers washed with brine, dried over Na₂SO₄ and filtered and concentrated. The residue obtained was purified by prep-HPLC to afford the (S)—N-((2S,3R)-3-((2-oxabicyclo[2.2.2]octan-4-yl)methoxy)-1-((3-(methylsulfonamido)-3-oxopropyl)amino)-1-oxobutan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (2.2 mg, 10%) as a white solid. LCMS m/z=918.2 [M+H]⁺; ¹H NMR (400 MHz, DMSO-d6) δ 7.94-7.77 (m, 2H), 7.61-7.55 (m, 2H), 7.35 (s, 2H), 5.36 (s, 2H), 4.52-4.39 (m, 2H), 4.21-3.87 (m, 6H), 3.78-3.41 (m, 8H), 3.27-3.24 (m, 4H), 3.12-3.04 (m, 1H), 2.58-2.49 (m, 2H), 1.98-2.02 (m, 3H), 1.63-1.60 (m, 4H), 1.46-1.40 (m, 3H), 1.20 (s, 3H), 1.04-1.02 (m, 2H).

### Synthesis of tert-butyl 4-((4-((S)-8-(((2S,3R)-3-((2-oxabicyclo[2.2.2]octan-4-yl)methoxy)-1-(4-(2-(methoxycarbonyl)phenyl)piperidin-1-yl)-1-oxobutan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)piperidine-1-carboxylate (I-65)

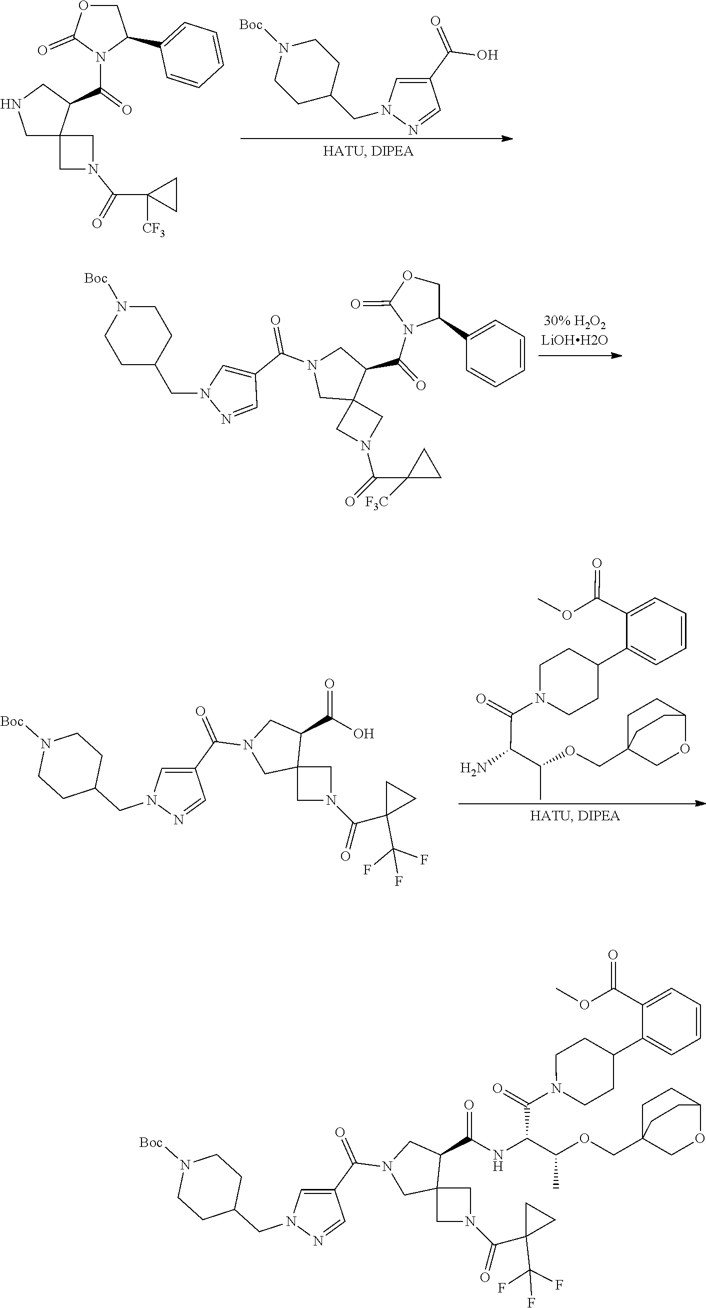

Step 1: tert-butyl 4-((4-((S)-8-((R)-2-oxo-4-phenyloxazolidine-3-carbonyl)-2-(1-(trifluoromethyl)cyclopropane-1-carbonyl)-2,6-diazaspiro[3.4]octane-6-carbonyl)-1H-pyrazol-1-yl)methyl)piperidine-1-carboxylate To a solution of 1-((1-(tert-butoxycarbonyl)piperidin-4-yl)methyl)-1H-pyrazole-4-carboxylic acid (900 mg, 2.9 mmol) in DCM (40 mL) was added HATU (1.1 g, 2.9 mmol) and the mixture was stirred at room temperature for 30 min. (R)-4-phenyl-3-((S)-2-(1-(trifluoromethyl)cyclopropane-1-carbonyl)-2,6-diazaspiro[3.4]octane-8-carbonyl)oxazolidin-2-one (1.3 g, 2.9 mmol) and DIPEA (1.1 g, 8.7 mmol) were added and stirring continued for 2 h. The mixture was diluted with water (100 mL) and extracted with EtOAc (150 mL×3). The combined organic layers were washed with brine, dried over Na₂SO₄, filtered and concentrated. The residue obtained was purified by column chromatography on silica gel (eluent: DCM:MeOH=50:1) to afford tert-butyl 4-((4-((S)-8-((R)-2-oxo-4-phenyloxazolidine-3-carbonyl)-2-(1-(trifluoromethyl)cyclopropane-1-carbonyl)-2,6-diazaspiro[3.4]octane-6-carbonyl)-1H-pyrazol-1-yl)methyl)piperidine-1-carboxylate (800 mg, 38%) as a yellow solid. LCMS m/z=629.1 [M+H-100]⁺; ¹H NMR (400 MHz, DMSO-d6) δ 8.15 (s, 1H), 7.79-7.73 (m, 1H), 7.34-7.07 (m, 5H), 5.46 (s, 1H), 4.80-4.70 (m, 1H), 4.42-3.98 (m, 8H), 3.95-3.74 (m, 6H), 2.74-2.58 (m, 2H), 2.08-1.91 (m, 1H), 1.45-1.35 (m, 12H), 1.23-1.13 (m, 3H), 1.11-0.97 (m, 2H).

Step 2: (S)-6-(1-((1-(tert-butoxycarbonyl)piperidin-4-yl)methyl)-1H-pyrazole-4-carbonyl)-2-(1-(trifluoromethyl)cyclopropane-1-carbonyl)-2,6-diazaspiro[3.4]octane-8-carboxylic acid To a solution of tert-butyl 4-((4-((S)-8-((R)-2-oxo-4-phenyloxazolidine-3-carbonyl)-2-(1-(trifluoromethyl)cyclopropane-1-carbonyl)-2,6-diazaspiro[3.4]octane-6-carbonyl)-1H-pyrazol-1-yl)methyl)piperidine-1-carboxylate (700 mg, 0.14 mmol) in a mixture of THE and water (3 mL/0.25 mL) at 0° C. was added a solution of lithium hydroxide monohydrate (84 mg, 0.28 mmol) and 30% H₂O₂ (280 mg, 0.35 mmol) in water (1 mL). The reaction mixture was stirred at 0° C. for 1 h then was diluted with water (15 mL) and extracted with EtOAc (30 mL). The aqueous layer was collected, acidified with 1M HCl to pH ~2 and extracted with EtOAc (80 mL×3). The combined organic layers were washed with brine, dried over Na₂SO₄, filtered and concentrated to afford (S)-6-(1-((1-(tert-butoxycarbonyl)piperidin-4-yl)methyl)-1H-pyrazole-4-carbonyl)-2-(1-(trifluoromethyl)cyclopropane-1-carbonyl)-2,6-diazaspiro[3.4]octane-8-carboxylic acid (500 mg, 89%) as a white solid. LCMS m/z=484.1 [M+H-100]⁺; ¹H NMR (400 MHz, DMSO-d6) δ 8.19 (d, J=4.6 Hz, 1H), 7.80 (d, J=14.8 Hz, 1H), 4.44-4.13 (m, 2H), 4.11-3.59 (m, 11H), 3.37-3.26 (m, 2H), 2.78-2.56 (m, 2H), 2.07-1.99 (m, 1H), 1.47-1.34 (m, 11H), 1.30-1.19 (m, 2H), 1.11-0.98 (m, 2H).

Step 3: tert-butyl 4-((4-((S)-8-(((2S,3R)-3-((2-oxabicyclo[2.2.2]octan-4-yl)methoxy)-1-(4-(2-(methoxycarbonyl)phenyl)piperidin-1-yl)-1-oxobutan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)piperidine-1-carboxylate I-65 To a solution of (S)-6-(1-((1-(tert-butoxycarbonyl)piperidin-4-yl)methyl)-1H-pyrazole-4-carbonyl)-2-(1-(trifluoromethyl)cyclopropane-1-carbonyl)-2,6-diazaspiro[3.4]octane-8-carboxylic acid (50 mg, 0.08 mmol) in DCM (3 mL) was added HATU (39 mg, 0.10 mmol) and the mixture stirred at room temperature for 5 mins. DIPEA (33 mg, 0.26 mmol) and methyl 2-(1-(O-((2-oxabicyclo[2.2.2]octan-4-yl)methyl)-L-threonyl)piperidin-4-yl)benzoate (46 mg, 0.10 mmol) were added and the reaction stirred for another 2 h. The mixture was diluted with water (30 mL) and extracted with DCM (40 mL×3). The combined organic layers were washed with brine, dried over Na₂SO₄, filtered and concentrated. The residue obtained was purified by prep-HPLC to afford tert-butyl 4-((4-((S)-8-(((2S,3R)-3-((2-oxabicyclo[2.2.2]octan-4-yl)methoxy)-1-(4-(2-(methoxycarbonyl)phenyl)piperidin-1-yl)-1-oxobutan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)piperidine-1-carboxylate (15 mg, 17%) as a white solid. LCMS m/z=910.3 [M+H-100]⁺; ¹H NMR (400 MHz, DMSO-d6) δ 8.33 (s, 1H), 8.17 (s, 1H), 7.80 (d, J=12.4 Hz, 1H), 7.70 (s, 1H), 7.55 (s, 1H), 7.36 (d, J=8.4 Hz, 2H), 4.91 (s, 1H), 4.56 (s, 1H), 4.14 (s, 3H), 4.02 (s, 4H), 3.88 (s, 4H), 3.84 (s, 3H), 3.75 (s, 1H), 3.60 (d, J=25.4 Hz, 6H), 3.46 (s, 2H), 3.12 (dd, J=23.6, 12.7 Hz, 3H), 2.59 (s, 1H), 1.99 (s, 1H), 1.82 (s, 5H), 1.56 (d, J=10.8 Hz, 6H), 1.41 (s, 2H), 1.38 (s, 9H), 1.24 (s, 2H), 1.16 (s, 2H), 1.04 (d, J=16.2 Hz, 7H).

### Synthesis of 2-((cyclohexylmethyl)((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)amino)-N-(methylsulfonyl)acetamide (I-70)

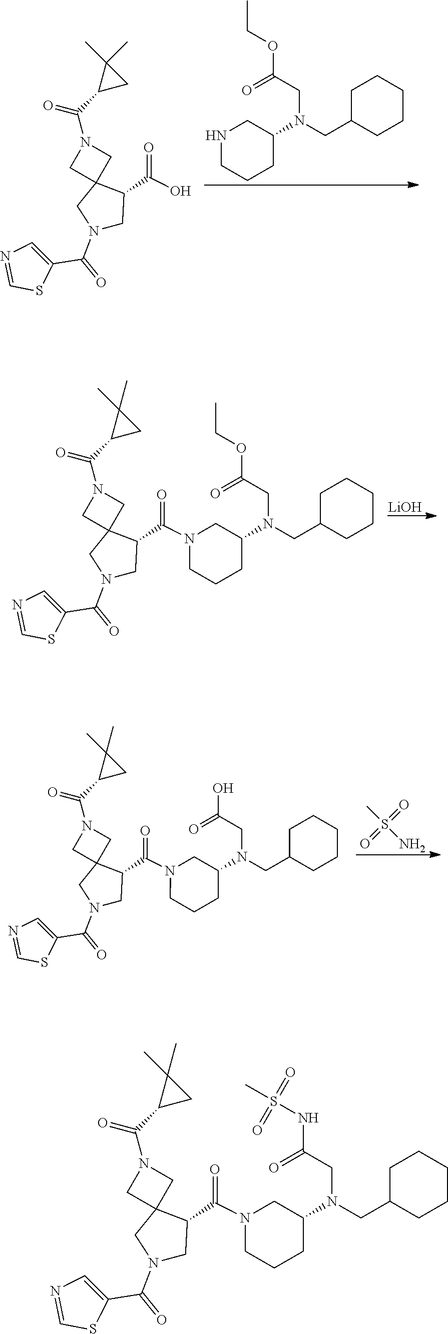

Step 1: ethyl N-(cyclohexylmethyl)-N—((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycinate To a solution of (S)-2-((S)-2,2-dimethylcyclopropane-1-carbonyl)-6-(thiazole-5-carbonyl)-2,6-diazaspiro[3.4]octane-8-carboxylic acid (300 mg, 0.8 mmol) in DCM (5 mL) and added HATU (376 mg, 1.0 mmol) and DIPEA (319 mg, 2.47 mmol) and the mixture stirred at room temperature for 30 min. Ethyl (R)—N-(cyclohexylmethyl)-N-(piperidin-3-yl)glycinate (279 mg, 1.0 mmol) was added and stirring continued for 2 h. The mixture was diluted with water (30 mL) and extracted with EtOAc (50 mL×3). The combined organic layers were washed with brine, dried over Na₂SO₄, filtered and concentrated. The residue was purified by RP-column to afford ethyl N-(cyclohexylmethyl)-N—((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycinate (130 mg, 25%) as a white solid. LCMS m/z=628.5[M+H]⁺.

Step 2: N-(cyclohexylmethyl)-N—((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycine I-70 To a solution of ethyl N-(cyclohexylmethyl)-N—((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycinate (130 mg, 0.2 mmol) in MeOH (1 mL) was added LiOH (17 mg, 0.4 mmol) and the reaction stirred at room temperature for 2 h. The mixture was diluted with water (20 mL) and the pH adjusted to 5 with 1 N HCl. The solvent was removed under reduced pressure and the residue obtained purified by RP-column to afford ethyl N-(cyclohexylmethyl)-N—((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycine (50 mg, 41%) as a white solid. LCMS m/z=600.2 [M+H]⁺; ¹HNMR (400 MHz, DMSO-d₆) δ 9.16 (s, 1H), 8.12 (s, 1H), 4.71-3.58 (m, 14H), 3.26 (s, 1H), 3.08 (s, 1H), 2.95 (s, 1H), 2.21 (s, 1H), 1.99-1.83 (m, 3H), 1.78-1.62 (m, 5H), 1.39 (d, J=56.7 Hz, 5H), 1.21-1.10 (m, 7H), 1.01 (d, J=30.1 Hz, 3H), 0.79 (s, 1H).

Step 3: 2-((cyclohexylmethyl)((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)amino)-N-(methylsulfonyl)acetamide To a solution of N-(cyclohexylmethyl)-N—((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)glycine (40 mg, 66.8 umol) in DCM (1 mL) was added HATU (30 mg, 80.1 umol) and DIPEA (25 mg, 200.3 umol) and the mixture stirred at room temperature for 30 min. Methanesulfonamide (7 mg, 73.4 mmol) was added and stirring continued overnight. The mixture was diluted with water (30 mL) and extracted with DCM (50 mL×2). The combined organic layers were washed with brine, dried over Na₂SO₄, filtered and concentrated. The residue was purified by prep-TLC (DCM:MeOH=15:1) to afford 2-((cyclohexylmethyl)((R)-1-((S)-2-((S)-2,2-dimethylcyclopropane-1-carbonyl)-6-(thiazole-5-carbonyl)-2,6-diazaspiro[3.4]octane-8-carbonyl)piperidin-3-yl)amino)-N-(methylsulfonyl)acetamide (7 mg, 16%) as a white solid. LCMS m/z=677.1 [M+H]⁺; ¹H NMR (400 MHz, CD₃OD) δ 9.16 (s, 1H), 8.09 (s, 1H), 4.62-3.76 (m, 11H), 3.64-3.48 (m, 2H), 3.25-2.51 (m, 8H), 2.15-2.01 (m, 1H), 1.92-1.36 (m, 10H), 1.35-1.09 (m, 9H), 1.07-0.88 (m, 3H), 0.83-0.72 (m, 1H).

### Building Blocks:

### Synthesis of methyl 5-(piperidin-4-yl)picolinate

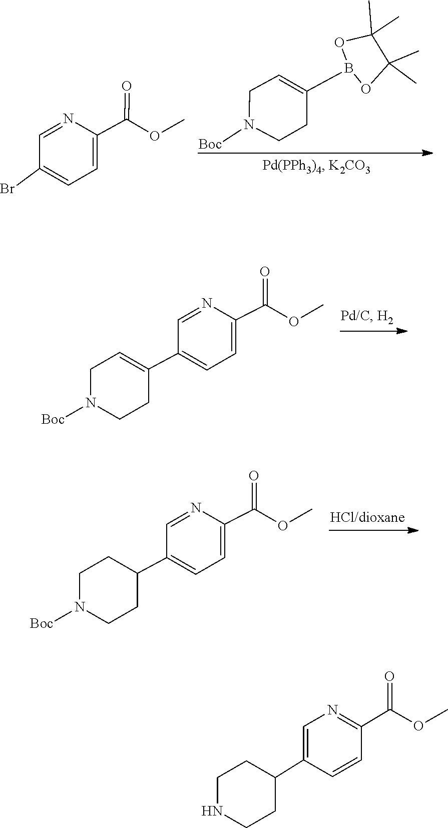

Step 1: methyl 1′-(tert-butylcarboxy)-1′,2′,3′,6′-tetrahydro-[3,4′-bipyridine]-6-carboxylate To a solution of methyl 5-bromopicolinate (1.0 g, 4.63 mmol) in a mixture of dioxane (10 mL) and water (2 mL) was added tert-butyl 4-(4,4,5,5-tetramethyl-1,3,2-dioxaborolan-2-yl)-3,6-dihydropyridine-1 (2H)-carboxylate (1.7 g, 5.55 mmol), K₂CO₃ (1.3 g, 9.26 mmol) and Pd(PPh₃)₄ (535 mg, 0.46 mol). The reaction was heated at 100° C. under N₂ atmosphere for 3 h then was diluted with water (50 mL) and extracted with EtOAc (100 mL×3). The combined organic layers were dried over Na₂SO₄, filtered and concentrated. The residue was purified by column chromatography on silica gel (eluent: Pet. Ether:EtOAc=4:1, v/v) to afford methyl 1′-(tert-butylcarboxy)-1′,2′,3′,6′-tetrahydro-[3,4′-bipyridine]-6-carboxylate (1.02 g, 68%) as a white solid. LCMS m/z=319.1 [M+H]⁺; ¹H NMR (400 MHz, DMSO-d₆) δ 8.82-8.80 (m, 1H), 8.04-7.98 (m, 2H), 6.46 (s, 1H), 4.07-4.03 (m, 2H), 3.88 (s, 3H), 3.56 (t, J=5.6 Hz, 2H), 2.55-2.51 (m, 2H), 1.43 (s, 9H).

Step 2: methyl 5-(1-(tert-butoxycarbonyl)piperidin-4-yl)picolinate To a solution of methyl 1′-(tert-butylcarboxy)-1′,2′,3′,6′-tetrahydro-[3,4′-bipyridine]-6-carboxylate (1.0 g, 3.14 mmol) in MeOH (8 mL) was added 10% Pd/C (400 mg, 40%) and the reaction heated at 50° C. under a H₂ atmosphere for 2 h. The catalyst was removed by filtration through Celite and the filtrate concentrated to afford the crude methyl 5-(1-(tert-butoxycarbonyl)piperidin-4-yl)picolinate (988 mg, 98%) as a black oil, which was used in the next step directly. LCMS m/z=321.1 [M+H]⁺; ¹H NMR (400 MHz, DMSO-d6) δ 8.63 (d, J=2.2 Hz, 1H), 8.00-7.97 (m, 1H), 7.89-7.86 (m, 1H), 4.14-4.04 (m, 2H), 3.86 (s, 3H), 2.90-2.76 (m, 3H), 1.82-1.74 (m, 2H), 1.59-1.49 (m, 2H), 1.42 (s, 9H).

Step 3: methyl 5-(piperidin-4-yl)picolinate To a solution of methyl 5-(1-(tert-butoxycarbonyl)piperidin-4-yl)picolinate (140 mg, 0.43 mmol) in DCM (1 mL) was added 4M HCl in dioxane (1 mL). The mixture was stirred at room temperature for 2 h then the solvent was removed under vacuum to afford methyl 5-(piperidin-4-yl)picolinate (96 mg, 100%) as a yellow oil. LCMS m/z=221.2 [M+H]⁺.

### Synthesis of methyl 4-(piperidin-4-yl)-2-(trifluoromethyl)benzoate

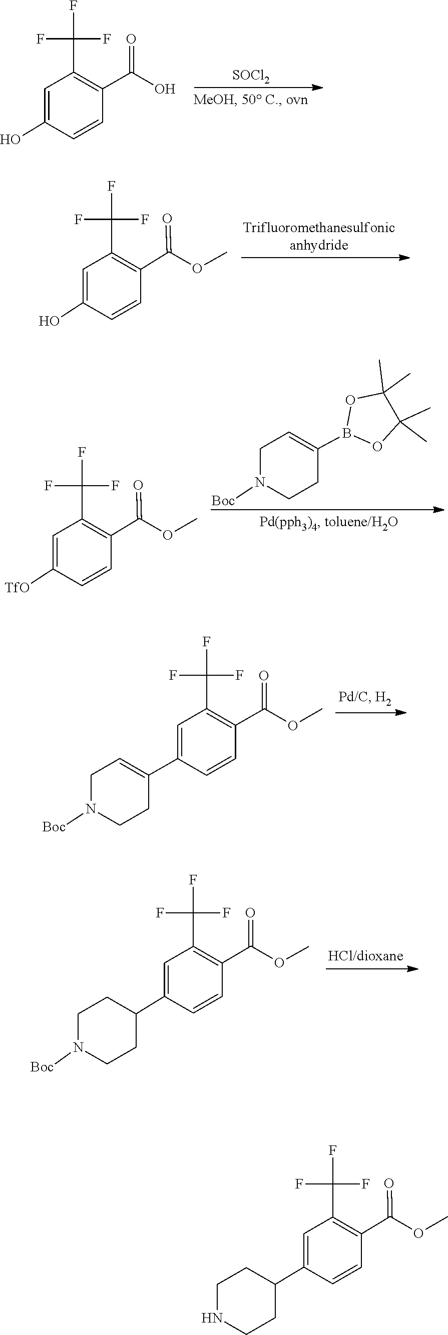

Step 1: methyl 4-hydroxy-2-(trifluoromethyl)benzoate To a solution of 4-hydroxy-2-(trifluoromethyl)benzoic acid (2 g, 9.7 mmol) in MeOH (30 mL) at 0° C. was added SOCl₂ (1.5 mL). The reaction was heated at 50° C. for 4 days then was quenched with water (100 mL) and extracted with EtOAc (100 mL×5). The combined organic layers were dried over Na₂SO₄, filtered and concentrated to afford methyl 4-hydroxy-2-(trifluoromethyl)benzoate (1.7 g, 81%) as a white solid. ¹H NMR (400 MHz, DMSO-d6) δ 7.78 (d, J=8.4 Hz, 1H), 7.18 (d, J=2.4 Hz, 1H), 7.09 (dd, J=8.8, 2.4 Hz, 1H), 3.80 (s, 3H).

Step 2: methyl 2-(trifluoromethyl)-4-(((trifluoromethyl)sulfonyl)oxy)benzoate To a solution of methyl 4-hydroxy-2-(trifluoromethyl)benzoate (1.7 g, 7.7 mmol) in DCM (17 mL) was added pyridine (1.3 g, 16.9 mmol) and trifluoromethanesulfonic anhydride (2.4 g, 8.5 mmol). The reaction was stirred at room temperature for 3 h then was quenched with water (100 mL) and extracted with EtOAc (100 mL×3). The combined organic layers were dried over Na₂SO₄, filtered and concentrated to afford methyl 2-(trifluoromethyl)-4-(((trifluoromethyl)sulfonyl)oxy)benzoate (2.2 g, 81%). 1H NMR (400 MHz, DMSO-d6) δ 8.16 (d, J=2.4 Hz, 1H), 8.08 (d, J=8.6 Hz, 1H), 8.01 (dd, J=8.8, 2.4 Hz, 1H), 3.90 (s, 3H).

Step 3: tert-butyl 4-(4-(methoxycarbonyl)-3-(trifluoromethyl)phenyl)-3,6-dihydropyridine-1 (2H)-carboxylate To a solution of methyl 2-(trifluoromethyl)-4-(((trifluoromethyl)sulfonyl)oxy)benzoate (2.2 g, 6.3 mmol) in a mixture of toluene and water (15 mL/3 mL) under a N₂ atmosphere was added tert-butyl 4-(4,4,5,5-tetramethyl-1,3,2-dioxaborolan-2-yl)-3,6-dihydropyridine-1 (2H)-carboxylate (2.3 g, 7.5 mmol), Pd(PPh₃)₄ (721 mg, 0.6 mmol) and K₂CO₃ (1.7 g, 12.5 mmol) and the reaction heated at 100° C. overnight. The mixture was cooled to room temperature, diluted with water (150 mL) and extracted with EtOAc (100 mL×3). The combined organic layers were washed with brine, dried over Na₂SO₄, filtered and concentrated. The residue obtained was purified by column chromatography (Petroleum ether:EtOAc=15/1, v/v) to afford tert-butyl 4-(4-(methoxycarbonyl)-3-(trifluoromethyl)phenyl)-3,6-dihydropyridine-1 (2H)-carboxylate (1.7 g, 70%) as colorless oil. LCMS m/z=330.2 [M+H-56]⁺; H NMR (400 MHz, DMSO-d6) δ 7.83 (d, J=2.8 Hz, 3H), 6.43 (s, 1H), 4.06-4.02 (m, 2H), 3.86 (s, 3H), 3.55 (t, J=5.8 Hz, 2H), 2.54-2.51 (m, 2H), 1.43 (s, 9H).

Step 4: tert-butyl 4-(4-(methoxycarbonyl)-3-(trifluoromethyl)phenyl)piperidine-1-carboxylate To a solution of tert-butyl 4-(4-(methoxycarbonyl)-3-(trifluoromethyl)phenyl)-3,6-dihydropyridine-1 (2H)-carboxylate (1.7 g, 4.4 mmol) in MeOH (15 mL) was added 10% Pd/C (680 mg) and the reaction stirred under a H₂ atmosphere for 12 h. The catalyst was removed by filtration though Celite and the filtrate concentrated to afford tert-butyl 4-(4-(methoxycarbonyl)-3-(trifluoromethyl)phenyl)piperidine-1-carboxylate (1.7 g, 100%). LCMS m/z=410.1[M+Na]⁺; ¹H NMR (400 MHz, DMSO-d6) δ 7.78 (d, J=8.0 Hz, 1H), 7.73 (d, J=1.6 Hz, 1H), 7.68 (dd, J=8.0, 1.8 Hz, 1H), 4.09 (d, J=13.2 Hz, 2H), 3.85 (s, 3H), 2.93-2.75 (m, 3H), 1.78 (dd, J=13.2, 3.2 Hz, 2H), 1.59-1.50 (m, 2H), 1.41 (s, 9H).

Step 5: methyl 4-(piperidin-4-yl)-2-(trifluoromethyl)benzoate To a solution of tert-butyl 4-(4-(methoxycarbonyl)-3-(trifluoromethyl)phenyl)piperidine-1-carboxylate (40 mg, 0.1 mmol) in DCM (1 mL) was added a solution of HCl in dioxane (4M, 1 mL). The reaction mixture was stirred at room temperature for 2 h then the solvent was removed under reduced pressure to afford methyl O-(cyclohexylmethyl)-L-threoninate (29 mg, 100%) which was used directly in the next step. LCMS m/z=288.1[M+H]⁺; ¹H NMR (400 MHz, DMSO-d6) δ 9.06 (d, J=48.4 Hz, 1H), 7.84 (d, J=7.8 Hz, 1H), 7.70-7.65 (m, 1H), 3.86 (s, 3H), 3.39-3.34 (m, 2H), 3.09-2.91 (m, 3H), 2.00-1.87 (m, 4H).

Table I-17: The compounds listed in Table I-17 were synthesized according to the procedure outlined for 4-nitrobenzyl ((2S,3R)-3-((2-oxabicyclo[2.2.2]octan-4-yl)methoxy)-1-oxo-1-(4-(4-(trifluoromethyl)benzyl)piperazin-1-yl)butan-2-yl)carbamate using the appropriate commercially available reagents.

TABLE I-17

| Compound | LCMS | ¹H NMR |
|---|---|---|
| methyl 3-((2S,3R)-3-((2-oxabicyclo[2.2.2]octan-4- | LCMS m/z = | ¹H NMR (400 MHz, |
| yl)methoxy)-2-((((4- | 508.2 [M + H]⁺ | DMSO-d₆) δ 8.24 (d, J = 2 |
| nitrobenzyl)oxy)carbonyl)amino)butanamido) | | Hz, 2H), 7.98 (t, J = 5.2 Hz, |
| propanoate | | 1H), 7.64 (d, J = 8.4 Hz, |
| 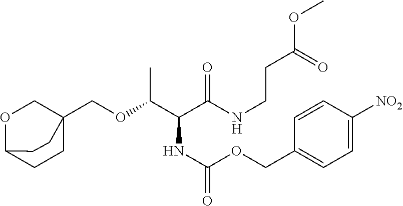 [SMILES: COC(=O)CCNC(=O)[C@@H](NC(=O)OCc1ccc([N+](=O)[O-])cc1)[C@@H](C)OCC12CCC(CC1)OC2] | | 2H), 7.19 (d, J = 9.2 Hz, 1H), 5.19 (s, 2H), 3.98 (dd, J₁ = 4.8 Hz, J₂ = 4.8 Hz, 1 H), 3.63-3.58 (m, 6H), 3.38-3.33 (m, 1H), 3.29- 3.15 (m, 2H), 3.10 (d, J = 9.2 Hz, 1H), 2.90 (d, J = 9.2 Hz, 1H), 2.46 (t, J = 6.8 Hz, 2H), 1.81-1.75 (m, 2H), 1.55-1.49 (m, 4H), 1.38- 1.32 (m, 2H), 1.00 (d, J = 6 Hz, 3H). |

The compounds listed in Table I-17 cont. were synthesized according
to the procedure outlined for (2S, 3R)-2-amino-3-(2-cyclohexylethoxy)-N-methylbutanamide
using the appropriate commercially available reagents.

| Compound | LCMS |
|---|---|
| methyl 3-((2S,3R)-3-((2-oxabicyclo[2.2.2]octan-4- | LCMS m/z = 329.2 [M + H]⁺ |
| yl)methoxy)-2-aminobutanamido)propanoate | |
| 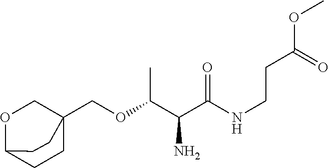 [SMILES: COC(=O)CCNC(=O)[C@@H](N)[C@@H](C)OCC12CCC(CC1)OC2] | |

### Synthesis of ethyl (R)-N-(cyclohexylmethyl)-N-(piperidin-3-yl)glycinate

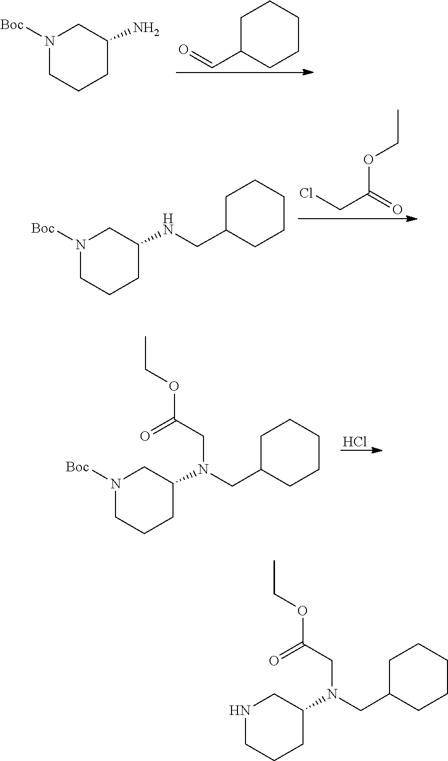

Step 1: tert-butyl (R)-3-((cyclohexylmethyl)amino)piperidine-1-carboxylate To a solution of tert-butyl (R)-3-aminopiperidine-1-carboxylate (1 g, 5.0 mmol) in DCE (10 mL) was added cyclohexanecarbaldehyde (560 mg, 5.0 mmol) and sodium triacetoxyborohydride (1.6 g, 7.5 mmol). The reaction mixture was stirred at room temperature overnight then was diluted with water (30 mL) and extracted with EtOAc (50 mL×3). The combined organic layers were washed with brine, dried over Na₂SO₄, filtered and concentrated to afford tert-butyl (R)-3-((cyclohexylmethyl)amino)piperidine-1-carboxylate (1.48 g, quant) as a white solid. LCMS m/z=297.4[M+H]⁺.

Step 2: tert-butyl (R)-3-((cyclohexylmethyl)(2-ethoxy-2-oxoethyl)amino)piperidine-1-carboxylate To a solution of tert-butyl (R)-3-((cyclohexylmethyl)amino)piperidine-1-carboxylate (1.48 g, 5.0 mmol) in DMF (10 mL) was added ethyl 2-chloroacetate (618 mg, 5 mmol) and K₂CO₃ (2 g, 15 mmol). The mixture was stirred at room temperature overnight then was diluted with water (30 mL) and extracted with EtOAc (50 mL×3). The combined organic layers were washed with brine, dried over Na₂SO₄, filtered and concentrated to afford tert-butyl (R)-3-((cyclohexylmethyl)(2-ethoxy-2-oxoethyl)amino)piperidine-1-carboxylate (1.9 g, quant) as an oil. LCMS m/z=383.0 [M+H]⁺.

Step 3: ethyl (R)—N-(cyclohexylmethyl)-N-(piperidin-3-yl)glycinate To a solution of tert-butyl (R)-3-((cyclohexylmethyl)(2-ethoxy-2-oxoethyl)amino)piperidine-1-carboxylate (1.9 g, 5.0 mmol) in DCM (10 mL) was added a solution of HCl in dioxane (4 M, 10 mL, 40 mmol. The reaction was stirred at room temperature for 4 h then the solvent was removed to afford ethyl (R)—N-(cyclohexylmethyl)-N-(piperidin-3-yl)glycinate (1.4 g, quant) as a yellow oil. LCMS m/z=283.2 [M+H]⁺.

### Synthesis of methyl 5-(1-(O-((2-oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)picolinate

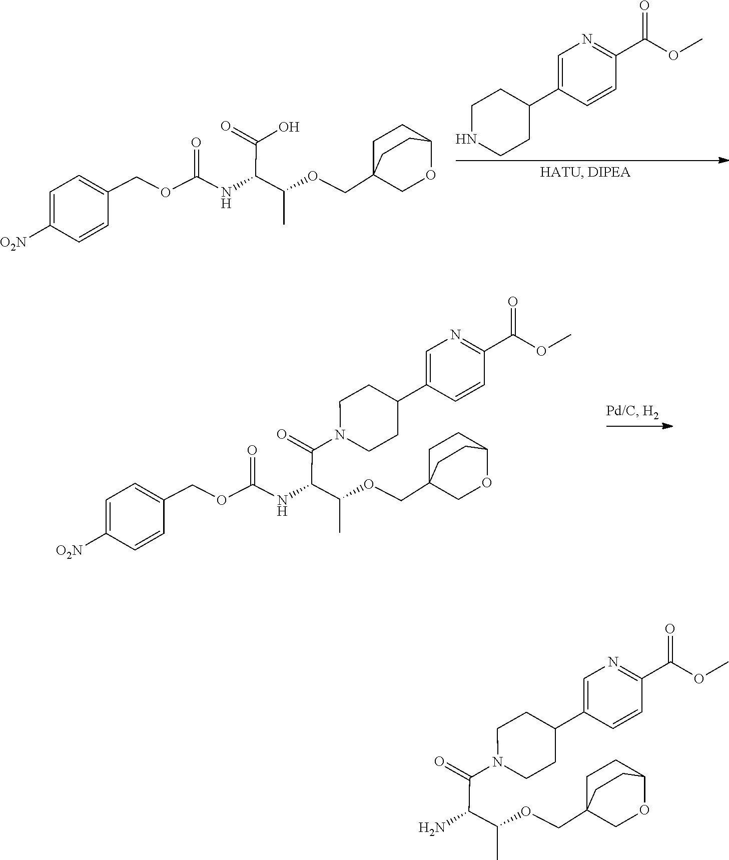

Step 1: methyl 5-(1-(O-((2-oxabicyclo[2.2.2]octan-4-yl)methyl)-N-(((4-nitrobenzyl)oxy)carbonyl)-L-threonyl)piperidin-4-yl)picolinate To a solution of O-((2-oxabicyclo[2.2.2]octan-4-yl)methyl)-N-(((4-nitrobenzyl)oxy)carbonyl)-L-threonine (160 mg, 0.38 mmol) in DCM (2 mL) was added HATU (187 mg, 0.49 mmol) and DIPEA (147 mg, 1.14 mmol) and the mixture stirred at room temperature for 30 min. Methyl 5-(piperidin-4-yl)picolinate (83 mg, 0.38 mmol) was added and stirring continued for 2 h. The mixture was diluted with water (20 mL) and extracted with DCM (50 mL×3). The combined organic layers were washed with brine, dried over Na₂SO₄, filtered and concentrated. The residue obtained was purified by prep-TLC (eluent: DCM:MeOH=20:1) to afford methyl 5-(1-(O-((2-oxabicyclo[2.2.2]octan-4-yl)methyl)-N-(((4-nitrobenzyl)oxy)carbonyl)-L-threonyl)piperidin-4-yl)picolinate (94 mg, 40%) as a colorless oil. LCMS m/z=625.2 [M+H]⁺; ¹H NMR (400 MHz, DMSO-d₆) δ 8.60 (d, J=14.9 Hz, 1H), 8.19 (dd, J=36.1, 8.3 Hz, 2H), 8.03-7.96 (m, 1H), 7.87-7.76 (m, 1H), 7.65-7.58 (m, 2H), 7.55 (s, 1H), 5.18 (s, 2H), 4.57-4.48 (m, 2H), 4.19 (s, 1H), 3.87 (s, 3H), 3.64-3.57 (m, 2H), 3.52-3.39 (m, 5H), 3.15 (d, J=9.4 Hz, 1H), 2.99 (s, 1H), 1.92-1.41 (m, 12H), 1.10-1.02 (m, 3H).

Step 2: methyl 5-(1-(O-((2-oxabicyclo[2.2.2]octan-4-yl)methyl)-L-threonyl)piperidin-4-yl)picolinate To a solution of methyl 5-(1-(O-((2-oxabicyclo[2.2.2]octan-4-yl)methyl)-N-(((4-nitrobenzyl)oxy)carbonyl)-L-threonyl)piperidin-4-yl)picolinate (50 mg, 0.08 mmol) in MeOH (1 mL) was added 10% Pd/C (20 mg, 40%) and the reaction heated at 50° C. under a H₂ atmosphere for 1 h. The catalyst was removed by filtration through Celite and concentrated to afford methyl 5-(1-(O-((2-oxabicyclo[2.2.2]octan-4-yl)methyl)-L-threonyl)piperidin-4-yl)picolinate (35 mg, 100%) as a black oil, which was used directly in the next step. LCMS m/z=446.3 [M+H]⁺; ¹H NMR (400 MHz, DMSO-d₆) δ 8.62 (d, J=2.2 Hz, 1H), 8.00 (d, J=8.0 Hz, 1H), 7.84 (d, J=8.2 Hz, 1H), 6.81 (d, J=7.8 Hz, 1H), 6.46 (d, J=8.2 Hz, 1H), 4.57 (s, 1H), 4.21 (s, 1H), 3.87 (s, 3H), 3.67-3.37 (m, 8H), 3.14 (s, 1H), 2.99 (s, 1H), 1.84 (s, 4H), 1.59-1.35 (m, 8H), 1.09-0.98 (m, 3H).

### Synthesis of Methyl 2-(1-(cyclohexylmethyl)piperazin-2-yl)acetate

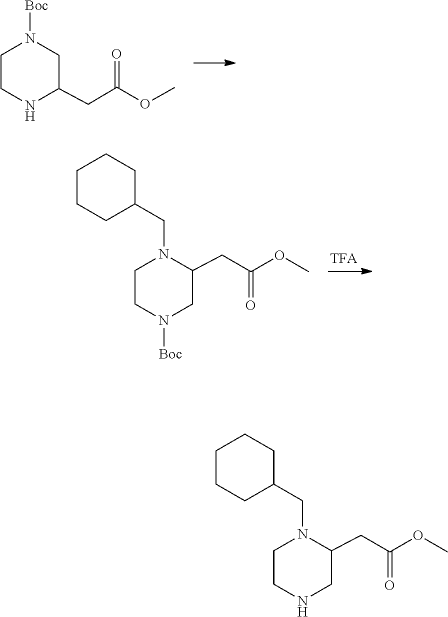 [SMILES: COC(=O)CC1CN(C(=O)OC(C)(C)C)CCN1 ; COC(=O)CC1CN(C(=O)OC(C)(C)C)CCN1CC1CCCCC1 ; COC(=O)CC1CNCCN1CC1CCCCC1]

Step 1: tert-butyl 4-(cyclohexylmethyl)-3-(2-methoxy-2-oxoethyl)piperazine-1-carboxylate To a solution of tert-butyl 3-(2-methoxy-2-oxoethyl)piperazine-1-carboxylate (250 mg, 1 mmol) and cyclohexanecarbaldehyde (112 mg, 1.32 mmol) in DCE (4 mL) was added sodium triacetoxyborohydride (318 mg, 1.5 mmol) and the reaction stirred at room temperature for 5 h. The mixture was diluted with water (30 mL) and extracted with DCM (30 mL×2). The combined organic layers were washed with brine, dried over Na₂SO₄, filtered and concentrated to afford tert-butyl 4-(cyclohexylmethyl)-3-(2-methoxy-2-oxoethyl)piperazine-1-carboxylate (240 mg, 91%) as a yellow oil. ¹H NMR (400 MHz, DMSO-d6) δ 3.60 (s, 3H), 3.19 (dd, J=13.2, 7.8 Hz, 1H), 3.01 (dd, J=9.6, 6.4 Hz, 1H), 2.86 (d, J=8.0 Hz, 1H), 2.45 (d, J=4.0 Hz, 1H), 2.35-2.20 (m, 4H), 1.81 (d, J=4.8 Hz, 3H), 1.37 (s, 9H), 1.29 (td, J=8.8, 8.2, 2.4 Hz, 6H), 1.22-1.09 (m, 4H), 0.83 (d, J=12.4 Hz, 1H).

Step 2: methyl 2-(1-(cyclohexylmethyl)piperazin-2-yl)acetate To a solution of tert-butyl 4-(cyclohexylmethyl)-3-(2-methoxy-2-oxoethyl)piperazine-1-carboxylate (350 mg, 1 mmol) in DCM (4 mL) was added TFA (1 mL) and the reaction stirred at room temperature for 2 h. The solvent was removed under vacuum to afford 1-((1-benzyl-1H-pyrazol-4-yl)methyl)-5-(methylcarbamoyl)-1H-indole-3-carboxylic acid (250 mg, 100%) which was used directly in the next step. LCMS m/z=255.2 [M+H]⁺.

### Synthesis of methyl 4-(1-(O-((2-oxabicyclo[2.2.2]octan-4-yl)methyl)-L-threonyl)piperidin-4-yl)-2-(trifluoromethyl)benzoate

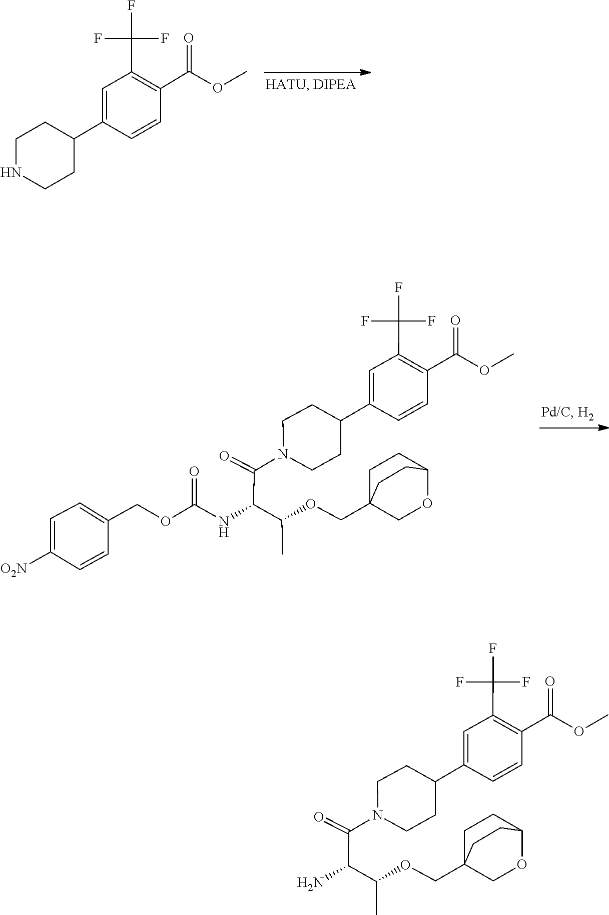 [SMILES: COC(=O)c1ccc(C2CCNCC2)cc1C(F)(F)F ; COC(=O)c1ccc(C2CCN(C(=O)[C@@H](NC(=O)OCc3ccc([N+](=O)[O-])cc3)[C@@H](C)OCC34CCC(CC3)OC4)CC2)cc1C(F)(F)F ; COC(=O)c1ccc(C2CCN(C(=O)[C@@H](N)[C@@H](C)OCC34CCC(CC3)OC4)CC2)cc1C(F)(F)F]

Step 1: 4-nitrobenzyl ((2S,3R)-3-((2-oxabicyclo[2.2.2]octan-4-yl)methoxy)-1-((4-fluorophenyl)sulfonamido)-1-oxobutan-2-yl)carbamate To a solution of 4-fluorobenzenesulfonamide (124 mg, 0.71 mmol) in DCM (4 mL) was added HATU (270 g, 0.71 mmol) and DIPEA (275 mg, 2.13 mmol) and the reaction stirred at room temperature for 30 min. O-((2-oxabicyclo[2.2.2]octan-4-yl)methyl)-N-(((4-nitrobenzyl)oxy)carbonyl)-L-threonine (300 mg, 0.71 mmol) was then added and stirring continued for 2 h. The mixture was diluted with water (50 mL) and extracted with DCM (100 mL×3). The combined organic layers were washed with brine, dried over Na₂SO₄, filtered and concentrated. The residue was purified by RP column (44% MeCN in water) to afford 4-nitrobenzyl ((2S,3R)-3-((2-oxabicyclo[2.2.2]octan-4-yl)methoxy)-1-((4-fluorophenyl)sulfonamido)-1-oxobutan-2-yl)carbamate (210 mg, 51%) as a yellow oil. LCMS m/z=580.1 [M+H]⁺; ¹H NMR (400 MHz, DMSO-d₆) δ 8.29-8.13 (m, 3H), 7.92-7.77 (m, 2H), 7.69-7.54 (m, 2H), 7.32-7.18 (m, 2H), 6.42 (s, 1H), 5.23-5.08 (m, 2H), 3.69-3.56 (m, 3H), 3.38-3.35 (m, 2H), 3.18-3.10 (m, 2H), 1.75 (q, J=8.4, 6.2 Hz, 2H), 1.45 (m, 2H), 1.32 (d, J=12.2 Hz, 2H), 1.22 (s, 3H), 0.94 (d, J=6.2 Hz, 2H).

Step 2: (2S,3R)-3-((2-oxabicyclo[2.2.2]octan-4-yl)methoxy)-2-amino-N-((4-fluorophenyl)sulfonyl)butanamide To a solution of 4-nitrobenzyl ((2S,3R)-3-((2-oxabicyclo[2.2.2]octan-4-yl)methoxy)-1-((4-fluorophenyl)sulfonamido)-1-oxobutan-2-yl)carbamate (210 mg, 0.36 mmol) in MeOH (4 mL) was added 10% Pd/C (105 mg) and the reaction heated at 50° C. under a H₂ atmosphere for 2 h. The catalyst was removed by filtration through Celite and the filtrate concentrated to afford (2S,3R)-3-((2-oxabicyclo[2.2.2]octan-4-yl)methoxy)-2-amino-N-((4-fluorophenyl)sulfonyl)butanamide (125 mg, 86%) as yellow oil which was used directly in the next step. LCMS m/z=401.0 [M+H]⁺.

### Synthesis of Ethyl (S)—N-(cyclohexylmethyl)-N-(piperidin-3-yl)glycinate

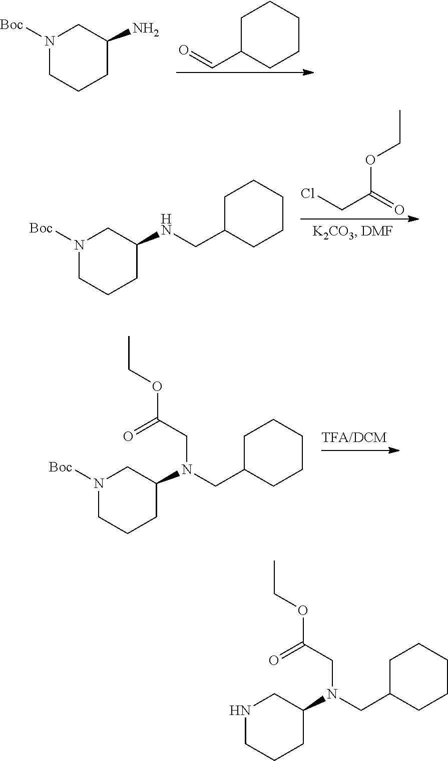

Step 1: tert-butyl (S)-3-((cyclohexylmethyl)amino)piperidine-1-carboxylate To a solution tert-butyl (S)-3-aminopiperidine-1-carboxylate (200 mg, 1 mmol) and cyclohexanecarbaldehyde (112 mg, 1 mmol) in DCE (4 mL) was added sodium triacetoxyborohydride (318 mg, 1.5 mmol) and the reaction stirred at room temperature for 5 h. The mixture was diluted with water (30 mL) and extracted with DCM (30 mL×2). The combined organic layers were washed with brine, dried over Na₂SO₄, filtered and concentrated to afford tert-butyl (S)-3-((cyclohexylmethyl)amino)piperidine-1-carboxylate (200 mg, 67%) which was used directly in the next step. LCMS m/z=297.3 [M+H]⁺.

Step 2: tert-butyl (S)-3-((cyclohexylmethyl)(2-ethoxy-2-oxoethyl)amino)piperidine-1-carboxylate To a solution of tert-butyl (S)-3-((cyclohexylmethyl)amino)piperidine-1-carboxylate (200 mg, 0.67 mmol) and ethyl 2-chloroacetate (81.7 mg, 0.67 mmol) in DMF (2 mL) was added K₂CO₃ (277 mg, 2.1 mmol) and catalytic amount KI. The reaction was stirred at room temperature for 2 h then was diluted with water (30 mL) and extracted with EtOAc (30 mL×3). The combined organic layers were washed with brine, dried over Na₂SO₄, filtered and concentrated to afford tert-butyl (S)-3-((cyclohexylmethyl)(2-ethoxy-2-oxoethyl)amino)piperidine-1-carboxylate (200 mg, 78%) which was used directly in the next step. LCMS m/z=383.3 [M+H]⁺.

Step 3: ethyl (S)—N-(cyclohexylmethyl)-N-(piperidin-3-yl)glycinate To a solution of tert-butyl (S)-3-((cyclohexylmethyl)(2-ethoxy-2-oxoethyl)amino)piperidine-1-carboxylate (200 mg, 0.52 mmol) in DCM (2 mL) was added TFA (1 mL) and the reaction stirred at room temperature for 2 h. The solvent was removed under vacuum to afford ethyl (S)—N-(cyclohexylmethyl)-N-(piperidin-3-yl)glycinate (146 mg, 100%) which was used directly in the next step. LCMS m/z=283.3 [M+H]⁺.

### Synthesis of 1-((1-(tert-butoxycarbonyl)piperidin-4-yl)methyl)-1H-pyrazole-4-carboxylic acid

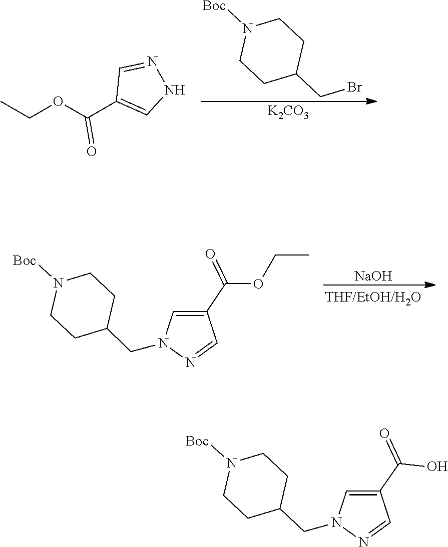

Step 1: tert-butyl 4-((4-(ethoxycarbonyl)-1H-pyrazol-1-yl)methyl)piperidine-1-carboxylate To a solution of ethyl 1H-pyrazole-4-carboxylate (4.5 g, 32 mmol) and tert-butyl 4-(bromomethyl)piperidine-1-carboxylate (8.9 g, 32 mmol) in MeCN (50 mL) was added K₂CO₃ (8.8 g, 64 mmol) and the reaction stirred at room temperature under a N₂ atmosphere overnight. The mixture was diluted with water (50 mL) and extracted with EtOAc (100 mL×3). The combined organic layers were washed with brine, dried over Na₂SO₄, filtered and concentrated. The residue obtained was purified by column chromatography on silica gel (eluent: Pet. Ether:EtOAc=1:1) to to afford tert-butyl 4-((4-(ethoxycarbonyl)-1H-pyrazol-1-yl)methyl)piperidine-1-carboxylate (7.8 g, 72%) as a white solid. LCMS m/z=360.1 [M+Na]⁺; ¹H NMR (400 MHz, DMSO-d6) δ 8.31 (s, 1H), 7.85 (s, 1H), 4.20 (q, J=7.0 Hz, 2H), 4.05 (d, J=7.2 Hz, 2H), 3.90 (d, J=13.2 Hz, 2H), 2.66 (s, 2H), 2.02-1.97 (m, 1H), 1.48-1.34 (m, 11H), 1.26 (t, J=7.2 Hz, 3H), 1.09-0.97 (m, 2H).

Step 2: 1-((1-(tert-butoxycarbonyl)piperidin-4-yl)methyl)-1H-pyrazole-4-carboxylic acid To a solution of tert-butyl 4-((4-(ethoxycarbonyl)-1H-pyrazol-1-yl)methyl)piperidine-1-carboxylate (1 g, 3 mmol) in a mixture of THE (8 mL) and water (2 ml) was added NaOH (118 mg, 6 mmol). The reaction mixture was stirred at room temperature for 4 h then was diluted with water (20 mL) and extracted with EtOAc (30 mL×2). The aqueous layers were collected, acidified with 1M HCl to pH~2 and extracted with EtOAc (50 mL×2). The combined organic layers were washed with brine, dried over Na₂SO₄, filtered and concentrated to afford 1-((1-(tert-butoxycarbonyl)piperidin-4-yl)methyl)-1H-pyrazole-4-carboxylic acid (900 mg, 98%) as a white solid. LCMS m/z=308.1 [M−H]⁻; ¹H NMR (400 MHz, DMSO-d6) δ 8.22 (s, 1H), 7.79 (s, 1H), 4.03 (d, J=7.2 Hz, 2H), 3.90 (d, J=13.2 Hz, 2H), 2.66 (s, 2H), 2.07-1.94 (m, 1H), 1.49-1.32 (m, 11H), 1.03 (qd, J=12.2, 4.4 Hz, 2H).

### Synthesis of (S)—N-((2S,3R)-1-(4-(1,3,4-oxadiazol-2-yl)piperidin-1-yl)-3-(cyclohexylmethoxy)-1-oxobutan-2-yl)-6-(1-benzyl-1H-pyrazole-4-carbonyl)-2-((S)-2,2-dimethylcyclopropane-1-carbonyl)-2,6-diazaspiro[3.4]octane-8-carboxamide

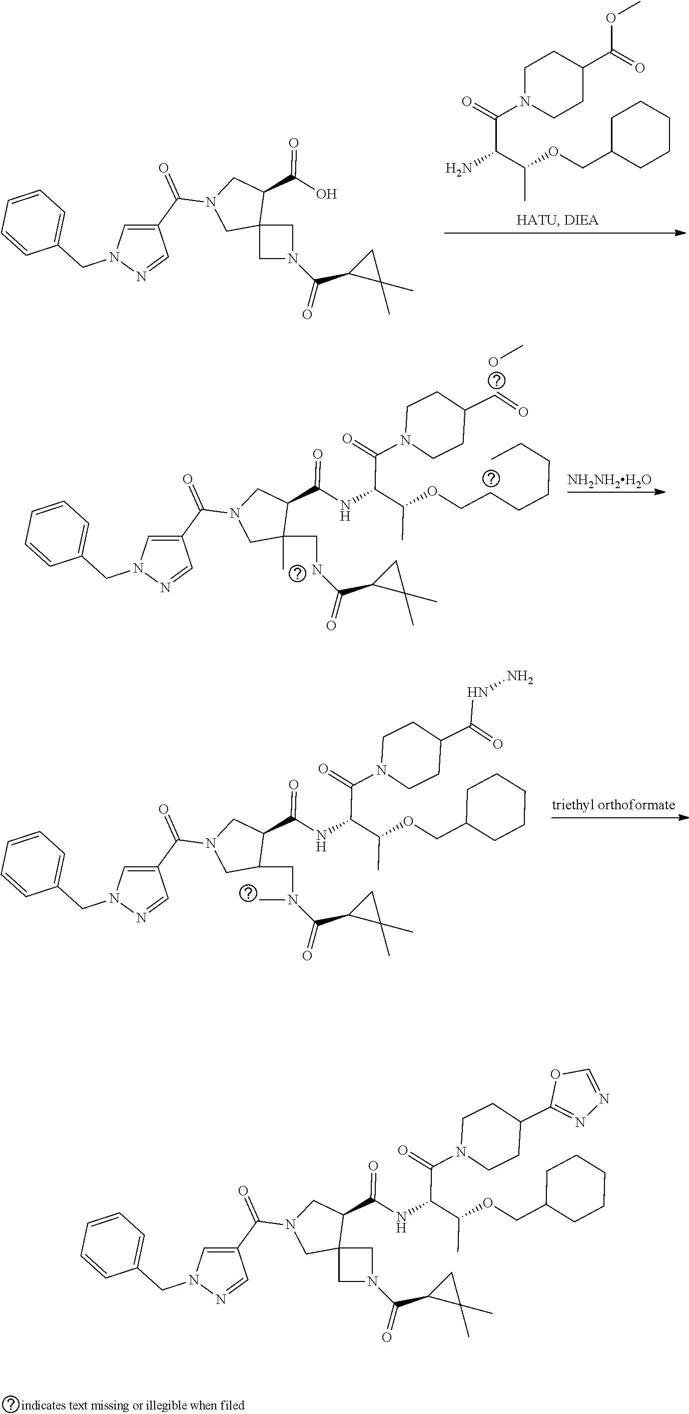

Step 1: methyl 1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-4-carboxylate To a solution of (S)-6-(1-benzyl-1H-pyrazole-4-carbonyl)-2-((S)-2,2-dimethylcyclopropane-1-carbonyl)-2,6-diazaspiro[3.4]octane-8-carboxylic acid (50 mg, 0.11 mmol) in DCM (2 mL) was added HATU (65 mg, 0.17 mmol) and DIPEA (44.4 mg, 0.34 mmol). The reaction mixture was stirred at room temperature for 30 min then methyl 1-(O-(cyclohexylmethyl)-L-threonyl)piperidine-4-carboxylate (47 mg, 0.14 mmol) was added and the mixture stirred for another 2 h. The reaction was diluted with water (20 mL) and extracted with DCM (50 mL×2). The combined organic layers were washed with brine, dried over Na₂SO₄, filtered and concentrated. The residue was purified by silica gel column (eluent: DCM:MeOH=20:1) to afford methyl 1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-4-carboxylate (30 mg, 35%) as a colorless oil. LCMS m/z=759.4 [M+H]⁺; ¹H NMR (400 MHz, DMSO-d6) δ 8.35 (d, J=14.4 Hz, 2H), 7.82 (d, J=15.8 Hz, 1H), 7.37-7.24 (m, 5H), 5.35 (s, 2H), 4.88 (s, 1H), 4.26-4.16 (m, 2H), 4.12-3.70 (m, 9H), 3.61 (s, 4H), 3.23 (s, 1H), 3.16 (s, 1H), 2.67 (s, 1H), 1.84 (s, 2H), 1.64 (d, J=13.2 Hz, 5H), 1.40 (m, 4H), 1.21-1.03 (m, 13H), 0.85 (d, J=7.2 Hz, 4H), 0.69-0.65 (m, 1H).

Step 2: (S)-6-(1-benzyl-1H-pyrazole-4-carbonyl)-N-((2S,3R)-3-(cyclohexylmethoxy)-1-(4-(hydrazinecarbonyl)piperidin-1-yl)-1-oxobutan-2-yl)-2-((S)-2,2-dimethylcyclopropane-1-carbonyl)-2,6-diazaspiro[3.4]octane-8-carboxamide To a solution of methyl 1-(N—((S)-6-(1-benzyl-1H-pyrazole-4-carbonyl)-2-((S)-2,2-dimethylcyclopropane-1-carbonyl)-2,6-diazaspiro[3.4]octane-8-carbonyl)-O-(cyclohexylmethyl)-L-threonyl)piperidine-4-carboxylate (10 mg, 0.01 mmol) in MeOH (1 mL) was added hydrazine hydrate (2.4 mg, 0.04 mmol). The reaction mixture was stirred at room temperature for 1 h then was diluted with water (10 mL) and extracted with DCM (20 mL×2). The mixture was concentrated to afford (S)-6-(1-benzyl-1H-pyrazole-4-carbonyl)-N-((2S,3R)-3-(cyclohexylmethoxy)-1-(4-(hydrazinecarbonyl)piperidin-1-yl)-1-oxobutan-2-yl)-2-((S)-2,2-dimethylcyclopropane-1-carbonyl)-2,6-diazaspiro[3.4]octane-8-carboxamide (4 mg, 40% yield) as a white solid which was used directly in the next step. LCMS m/z=759.30 [M+H]⁺.

Step 3: (S)—N-((2S,3R)-1-(4-(1,3,4-oxadiazol-2-yl)piperidin-1-yl)-3-(cyclohexylmethoxy)-1-oxobutan-2-yl)-6-(1-benzyl-1H-pyrazole-4-carbonyl)-2-((S)-2,2-dimethylcyclopropane-1-carbonyl)-2,6-diazaspiro[3.4]octane-8-carboxamide To a solution of (S)-6-(1-benzyl-1H-pyrazole-4-carbonyl)-N-((2S,3R)-3-(cyclohexylmethoxy)-1-(4-(hydrazinecarbonyl)piperidin-1-yl)-1-oxobutan-2-yl)-2-((S)-2,2-dimethylcyclopropane-1-carbonyl)-2,6-diazaspiro[3.4]octane-8-carboxamide (25 mg, 0.03 mmol) in triethyl orthoformate (0.5 mL) was added PPTS (0.6 mg, 0.003 mmol). The reaction mixture was heated at 150° C. in a sealed-tube for 1 h. The solvent was removed and the residue obtained purified by prep-HPLC to afford (S)—N-((2S,3R)-1-(4-(1,3,4-oxadiazol-2-yl)piperidin-1-yl)-3-(cyclohexylmethoxy)-1-oxobutan-2-yl)-6-(1-benzyl-1H-pyrazole-4-carbonyl)-2-((S)-2,2-dimethylcyclopropane-1-carbonyl)-2,6-diazaspiro[3.4]octane-8-carboxamide (2.2 mg, 9%) as a yellow solid. LCMS m/z=769.5 [M+H]⁺; ¹H NMR (400 MHz, Methanol-d₄) δ 8.87 (m, 1H), 8.21 (d, J=15.2 Hz, 1H), 7.96-7.88 (m, 1H), 7.39-7.25 (m, 5H), 5.38 (d, J=2.6 Hz, 2H), 4.99 (s, 1H), 4.60 (s, 1H), 4.47-3.71 (m, 11H), 3.35 (s, 3H), 3.21 (q, J=5.4, 5.0 Hz, 1H), 3.00 (s, 1H), 2.16 (m, 2H), 1.88 (d, J=6.6 Hz, 1H), 1.74 (m, 6H), 1.51 (dt, J=8.0, 3.6 Hz, 1H), 1.39-1.12 (m, 13H), 1.04 (t, J=4.6 Hz, 1H), 0.92 (m, 2H), 0.81-0.76 (m, 1H).

### Synthesis of (8S)—N-((3R)-3-((2-oxabicyclo[2.2.2]octan-4-yl)methoxy)-1-(methylamino)-1-oxobutan-2-yl)-2-((tetrahydrofuran-2-yl)methyl)-6-(thiazole-5-carbonyl)-2,6-diazaspiro[3.4]octane-8-carboxamide (I-59) and (8S)—N-((3R)-3-((1-(hydroxymethyl)cyclohex-3-en-1-yl)methoxy)-1-(methylamino)-1-oxobutan-2-yl)-2-((tetrahydrofuran-2-yl)methyl)-6-(thiazole-5-carbonyl)-2,6-diazaspiro[3.4]octane-8-carboxamide (I-58)

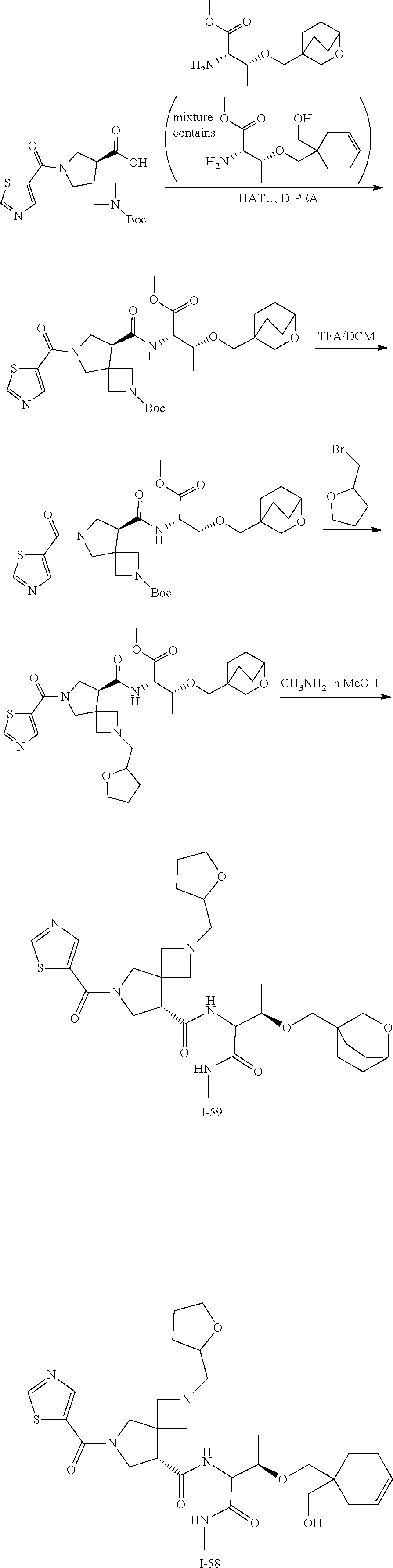

Step 1: tert-butyl (S)-8-(((2S,3R)-3-((2-oxabicyclo[2.2.2]octan-4-yl)methoxy)-1-methoxy-1-oxobutan-2-yl)carbamoyl)-6-(thiazole-5-carbonyl)-2,6-diazaspiro[3.4]octane-2-carboxylate: To a solution of (S)-2-(tert-butoxycarbonyl)-6-(thiazole-5-carbonyl)-2,6-diazaspiro[3.4]octane-8-carboxylic acid (428 mg, 1.17 mmol) in DCM (10 mL) was added HATU (532 mg, 1.40 mmol) and DIPEA (451 mg, 3.50 mmol). The reaction mixture was stirred at room temperature for 30 min then methyl O-((2-oxabicyclo[2.2.2]octan-4-yl)methyl)-L-threoninate (300 mg, 1.17 mmol) was added and the mixture stirred at room temperature for another 2 h. The mixture was diluted with water (30 mL) and extracted with DCM (50 mL×2). The combined organic layers were washed with brine, dried over Na₂SO₄, filtered and concentrated. The residue was purified by prep-TLC (eluent: DCM:MeOH=30:1) to afford tert-butyl (S)-8-(((2S,3R)-3-((2-oxabicyclo[2.2.2]octan-4-yl)methoxy)-1-methoxy-1-oxobutan-2-yl)carbamoyl)-6-(thiazole-5-carbonyl)-2,6-diazaspiro[3.4]octane-2-carboxylate (220 mg, 31%) as a yellow oil. LCMS m/z=607.3 [M+H]⁺; ¹H NMR (400 MHz, DMSO-d₆) δ 9.25 (s, 1H), 8.50-8.34 (m, 2H), 5.58 (dd, J=12.3 Hz, 2H), 4.53 (d, J=9.1 Hz, 1H), 4.30 (d, 1H), 4.12-4.03 (m, 2H), 3.89 (s, 3H), 3.81 (d, J=11.0 Hz, 2H), 3.68-3.64 (m, 3H), 3.57-3.48 (m, 2H), 3.28-3.10 (m, 2H), 1.96-1.86 (m, 2H), 1.81-1.70 (m, 2H), 1.61-1.48 (m, 2H), 1.36 (d, J=5.4 Hz, 13H), 1.11 (d, J=2.1 Hz, 1H), 1.08-1.03 (m, 3H).

Step 2: methyl O-((2-oxabicyclo[2.2.2]octan-4-yl)methyl)-N—((S)-6-(thiazole-5-carbonyl)-2,6-diazaspiro[3.4]octane-8-carbonyl)-L-threoninate To a solution of tert-butyl (S)-8-(((2S,3R)-3-((2-oxabicyclo[2.2.2]octan-4-yl)methoxy)-1-methoxy-1-oxobutan-2-yl)carbamoyl)-6-(thiazole-5-carbonyl)-2,6-diazaspiro[3.4]octane-2-carboxylate (220 mg, 0.36 mmol) in DCM (4 mL) was added TFA (1 mL). The mixture was stirred at room temperature for 2 h then concentrated to afford methyl O-((2-oxabicyclo[2.2.2]octan-4-yl)methyl)-N—((S)-6-(thiazole-5-carbonyl)-2,6-diazaspiro[3.4]octane-8-carbonyl)-L-threoninate (200 mg, 100%) as a yellow oil which was used directly in the next step. LCMS m/z=507.2 [M+H]⁺.

Step 3: methyl O-((2-oxabicyclo[2.2.2]octan-4-yl)methyl)-N-((8S)-2-((tetrahydrofuran-2-yl)methyl)-6-(thiazole-5-carbonyl)-2,6-diazaspiro[3.4]octane-8-carbonyl)-L-threoninate: To a solution of methyl O-((2-oxabicyclo[2.2.2]octan-4-yl)methyl)-N—((S)-6-(thiazole-5-carbonyl)-2,6-diazaspiro[3.4]octane-8-carbonyl)-L-threoninate (220 mg, 0.43 mmol) in DMF (6 mL) was added 2-(bromomethyl)tetrahydrofuran (144 mg, 0.86 mmol) and K₂CO₃ (119 mg, 0.86 mmol). The reaction was heated at 50° C. under a N₂ atmosphere overnight then diluted with water (30 mL) and extracted with EtOAc (50 mL×3). The combined organic layers were washed with brine, dried over Na₂SO₄, filtered and concentrated. The residue was purified by prep-TLC (DCM/MeOH=10/1, v/v) to afford methyl O-((2-oxabicyclo[2.2.2]octan-4-yl)methyl)-N-((8S)-2-((tetrahydrofuran-2-yl)methyl)-6-(thiazole-5-carbonyl)-2,6-diazaspiro[3.4]octane-8-carbonyl)-L-threoninate (30 mg, 12%) as a colorless oil. LCMS m/z=591.20 [M+H]⁺.

Step 4: (8S)—N-((3R)-3-((2-oxabicyclo[2.2.2]octan-4-yl)methoxy)-1-(methylamino)-1-oxobutan-2-yl)-2-((tetrahydrofuran-2-yl)methyl)-6-(thiazole-5-carbonyl)-2,6-diazaspiro[3.4]octane-8-carboxamide (1-59); and (8S)—N-((3R)-3-((1-(hydroxymethyl)cyclohex-3-en-1-yl)methoxy)-1-(methylamino)-1-oxobutan-2-yl)-2-((tetrahydrofuran-2-yl)methyl)-6-(thiazole-5-carbonyl)-2,6-diazaspiro[3.4]octane-8-carboxamide (1-58) A mixture of methyl O-((2-oxabicyclo[2.2.2]octan-4-yl)methyl)-N-((8S)-2-((tetrahydrofuran-2-yl)methyl)-6-(thiazole-5-carbonyl)-2,6-diazaspiro[3.4]octane-8-carbonyl)-L-threoninate (30 mg, 0.05 mmol) in a methanolic solution of MeNH₂ (40%, 4 mL) was heated at 60° C. for 2 h. The mixture was concentrated under reduced pressure and the residue obtained purified by prep-TLC (DCM/MeOH=10/1, v/v) and prep-HPLC to afford (8S)—N-((3R)-3-((2-oxabicyclo[2.2.2]octan-4-yl)methoxy)-1-(methylamino)-1-oxobutan-2-yl)-2-((tetrahydrofuran-2-yl)methyl)-6-(thiazole-5-carbonyl)-2,6-diazaspiro[3.4]octane-8-carboxamide (4 mg, 13%) as a gray solid. LCMS m/z=590.40 [M+H]⁺; ¹H NMR (400 MHz, Methanol-d₄) δ 9.16 (s, 1H), 8.35 (d, J=6.4 Hz, 1H), 4.64-4.54 (m, 2H), 4.36-4.27 (m, 1H), 4.07 (dd, J=19.9, 10.4 Hz, 2H), 3.97-3.81 (m, 5H), 3.72 (s, 4H), 3.55 (d, J=8.0 Hz, 2H), 3.49 (d, J=8.1 Hz, 2H), 2.78-2.68 (m, 5H), 2.05-1.95 (m, 3H), 1.92-1.84 (m, 2H), 1.69-1.62 (m, 3H), 1.55-1.48 (m, 3H), 1.32-1.28 (m, 2H), 1.19 (d, J=6.5 Hz, 3H). (8S)—N-((3R)-3-((1-(hydroxymethyl)cyclohex-3-en-1-yl)methoxy)-1-(methylamino)-1-oxobutan-2-yl)-2-((tetrahydrofuran-2-yl)methyl)-6-(thiazole-5-carbonyl)-2,6-diazaspiro[3.4]octane-8-carboxamide (4 mg, gray solid, 13.3% yield). LCMS m/z=590.45[M+H]⁺; ¹H NMR (400 MHz, Methanol-d₄) δ 9.15 (d, J=4.7 Hz, 1H), 8.35 (d, J=9.9 Hz, 1H), 7.33 (d, J=4.4 Hz, 1H), 5.62 (d, J=17.1 Hz, 2H), 4.58 (s, 2H), 4.35 (d, 1H), 4.12-3.99 (m, 2H), 3.95-3.86 (m, 3H), 3.84-3.75 (m, 2H), 3.72-3.68 (m, 1H), 3.54-3.47 (m, 2H), 3.46-3.41 (m, 3H), 2.74 (d, J=13.9 Hz, 3H), 2.69-2.56 (m, 2H), 2.05-1.97 (m, 3H), 1.92-1.85 (m, 2H), 1.82 (s, 1H), 1.58-1.51 (m, 2H), 1.42 (s, 1H), 1.33 (s, 2H), 1.29 (q, J=3.6, 2.8 Hz, 2H), 1.22 (d, J=6.3 Hz, 3H).

### Building Blocks:

### methyl 1-(O-(cyclohexylmethyl)-L-threonyl)piperidine-4-carboxylate

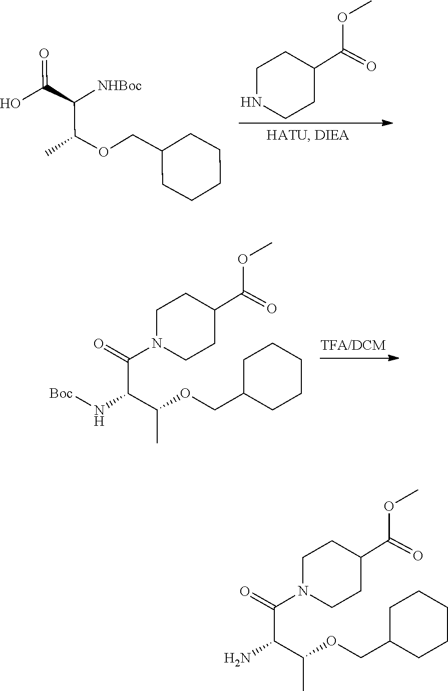

Step 1: methyl 1-(N-(tert-butoxycarbonyl)-O-(cyclohexylmethyl)-L-threonyl)piperidine-4-carboxylate To a solution of N-(tert-butoxycarbonyl)-O-(cyclohexylmethyl)-L-threonine (150 mg, 0.45 mmol) in DCM (5 mL) was added HATU (271 mg, 0.71 mmol) and DIPEA (185 mg, 1.43 mmol). The reaction mixture was stirred at room temperature for 30 min then methyl piperidine-4-carboxylate (82 mg, 0.57 mmol) was added and the mixture was stirred for another 2 h. The reaction was diluted with water (15 mL) and extracted with DCM (30 mL×2). The combined organic layers was washed with brine, dried over Na₂SO₄, filtered and concentrated. The residue was purified by silica gel column (eluent: Pet.Ether:EtOAc=3:1) to afford methyl 1-(N-(tert-butoxycarbonyl)-O-(cyclohexylmethyl)-L-threonyl)piperidine-4-carboxylate (200 mg, 95%) as a colorless oil. LCMS m/z=441.3 [M+H]⁺.

Step 2: methyl 1-(O-(cyclohexylmethyl)-L-threonyl)piperidine-4-carboxylate To a solution of methyl 1-(N-(tert-butoxycarbonyl)-O-(cyclohexylmethyl)-L-threonyl)piperidine-4-carboxylate (200 mg, 0.36 mmol) in DCM (2 mL) was added TFA (1 mL). The mixture was stirred at room temperature for 2 h then was concentrated to afford methyl 1-(O-(cyclohexylmethyl)-L-threonyl)piperidine-4-carboxylate (154 mg, 100%) as a yellow oil which was used directly in the next step.

Table I-18: The compounds listed in Table I-18 were synthesized according to the procedures outlined directly above for (S)—N-((2S,3R)-1-(4-(1,3,4-oxadiazol-2-yl)piperidin-1-yl)-3-(cyclohexylmethoxy)-1-oxobutan-2-yl)-6-(1-benzyl-1H-pyrazole-4-carbonyl)-2-((S)-2,2-dimethylcyclopropane-1-carbonyl)-2,6-diazaspiro[3.4]octane-8-carboxamide or (8S)—N-((3R)-3-((2-oxabicyclo[2.2.2]octan-4-yl)methoxy)-1-(methylamino)-1-oxobutan-2-yl)-2-((tetrahydrofuran-2-yl)methyl)-6-(thiazole-5-carbonyl)-2,6-diazaspiro[3.4]octane-8-carboxamide (I-59); and (8S)—N-((3R)-3-((1-(hydroxymethyl)cyclohex-3-en-1-yl)methoxy)-1-(methylamino)-1-oxobutan-2-yl)-2-((tetrahydrofuran-2-yl)methyl)-6-(thiazole-5-carbonyl)-2,6-diazaspiro[3.4]octane-8-carboxamide (1-58) using the appropriate commercially available reagents.

### Synthesis of 2-((4-((S)-8-(((2R,3R)-3-(2-oxabicyclo[2.2.2]octan-4-ylmethoxy)-1-(piperidin-4-yloxy)butan-2-yl)carbamoyl)-2-(1-(trifluoromethyl)cyclopropanecarbonyl)-2,6-diazaspiro[3.4]octane-6-carbonyl)-1H-pyrazol-1-yl)methyl)benzoic acid I-106

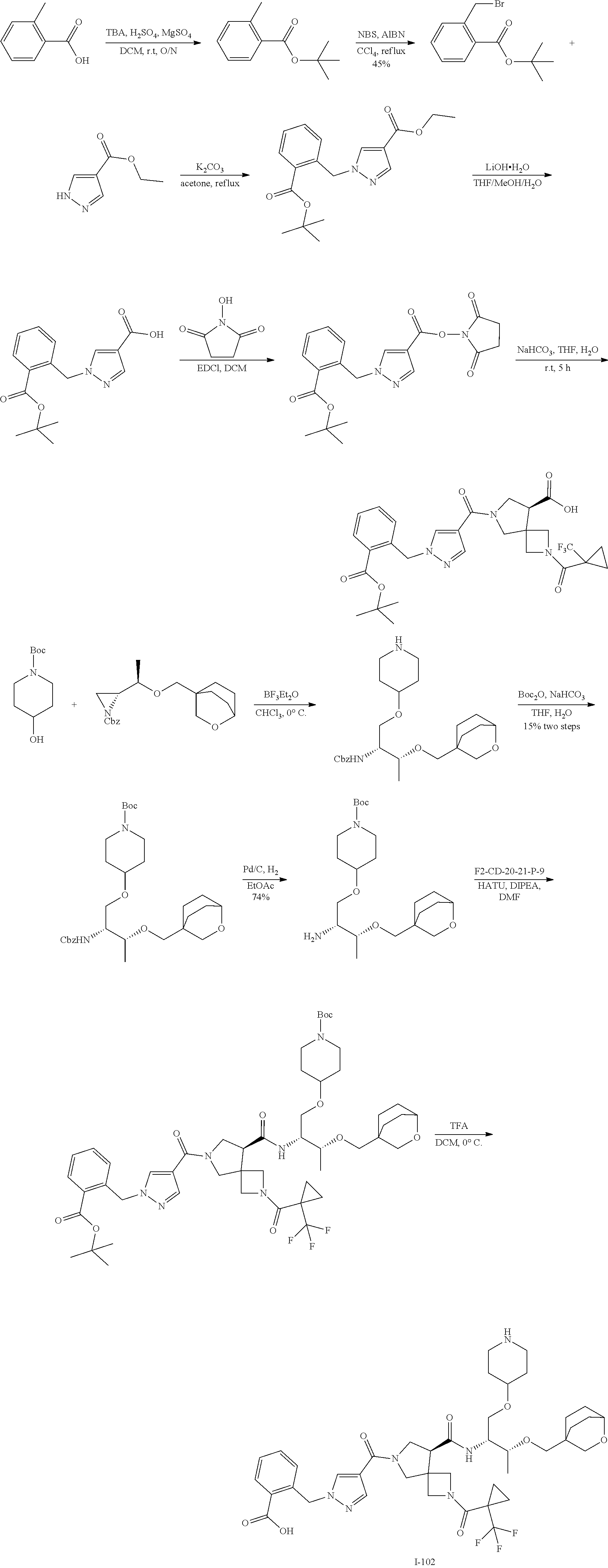

Tert-butyl 2-methylbenzoate: To a mixture of 2-methylbenzoic acid (10.000 g, 73.51 mmol) and magnesium sulfate (35.200 g, 292.40 mmol) in dichloromethane (100 mL) was added conc. sulfuric acid (7.200 g, 73.00 mmol) and tert-butanol (27.200 g, 267.50 mmol) under N₂. The resulting mixture was stirred at r.t overnight. The reaction mixture was poured into ice cold aqueous sodium hydroxide solution (500 mL) and extracted with dichloromethane (300 mL). The organic layer was collected, washed with brine (100 mL), dried over anhydrous sodium sulfate, and concentrated to give a crude residue which was purified by silica gel flash column chromatography using a 100% hexane gradient to afford tert-butyl 2-methylbenzoate (5.650 g, yield 40%) as a colorless oil. ¹HNMR (400 MHz, CDCl₃): δ 7.82-7.80 (m, 1H), 7.35-7.31 (m, 1H), 7.20 (t, J=8.0 Hz, 2H), 2.56 (s, 3H), 1.59 (s, 9H).

Tert-butyl 2-(bromomethyl)benzoate: To a solution of tert-butyl 2-methylbenzoate (5.650 g, 29.40 mmol) in carbon tetrachloride (200 mL) was added NBS (6.05 g, 34.00 mmol) and AIBN (1.03 g, 6.27 mmol), and the resulting mixture was refluxed under nitrogen atmosphere for 3 hours. The reaction mixture was poured into water (100 mL) and extracted with ethyl acetate (300 mL). The combined organic layers were washed with brine (100 mL), dried over anhydrous sodium sulfate, and concentrated to give a crude residue which was purified by silica gel flash column chromatography using a 100% hexane gradient to afford tert-butyl 2-(bromomethyl)benzoate (3.500 g, 44% yield) as a yellow oil. ¹HNMR (400 MHz, CDCl₃): δ 7.88-7.86 (m, 1H), 7.46-7.40 (m, 2H), 7.36-7.32 (m, 1H), 4.92 (s, 2H), 1.63 (s, 9H).

Ethyl 1-(2-(tert-butoxycarbonyl)benzyl)-1H-pyrazole-4-carboxylate: A mixture of tert-butyl 2-(bromomethyl)benzoate (2.000 g, 7.38 mmol), ethyl 1H-pyrazole-4-carboxylate (1.03 g, 7.40 mmol), and potassium carbonate (2.07 g, 15.00 mmol) in acetone (20 mL) was refluxed for 3 hours. The reaction mixture was poured into water (30 mL) and extracted with ethyl acetate (30 mL×2). The combined organic layers were washed with brine (30 mL), dried over anhydrous sodium sulfate, and concentrated to give a crude residue which was purified by silica gel flash column chromatography using a 0-10% ethyl acetate in hexane gradient to afford ethyl 1-(2-(tert-butoxycarbonyl)benzyl)-1H-pyrazole-4-carboxylate (2.050 g, yield 84%) as yellow oil. ¹HNMR (400 MHz, CDCl₃): δ 7.97-7.93 (m, 3H), 7.47-7.43 (m, 1H), 7.39-7.35 (m, 1H), 7.01-7.00 (m, 1H), 5.74 (s, 2H), 4.31-4.25 (m, 2H). 1.58 (s, 9H), 1.33 (t, J=8.0 Hz 3H).

1-(2-(Tert-butoxycarbonyl)benzyl)-1H-pyrazole-4-carboxylic acid: To a solution of ethyl 1-(2-(tert-butoxycarbonyl)benzyl)-1H-pyrazole-4-carboxylate (1.700 g, 5.14 mmol) in tetrahydrofuran (10 mL)-methanol (5 mL)-H₂O (5 mL) was added lithium hydroxide (0.281 g, 6.69 mmol). The resulting mixture was stirred at 60° C. for 1 h. The reaction mixture was concentrated, the residue was diluted with water (10 mL), and extracted with dichloromethane (10 mL). The aqueous layer was acidified to pH 3-4 with hydrochloric acid (2.0 N), and extracted with dichloromethane (15 mL×3). The combined organic layers were dried over anhydrous sodium sulfate, and concentrated to give which was purified by silica gel column chromatography using a 25% ethyl acetate in hexane gradient to afford 1-(2-(tert-butoxycarbonyl)benzyl)-1H-pyrazole-4-carboxylic acid (0.925 g, yield 60%) as a white solid. MS: [MH]⁺303.0

6-((1-(2-(Tert-butoxycarbonyl)benzyl)-1H-pyrazole-4-carbonyl)oxy)-2-(1-(trifluoromethyl)cyclopropane-1-carbonyl)-2,6-diazaspiro[3.5]nonane-9-carboxylic acid: A mixture of 1-(2-(tert-butoxycarbonyl)benzyl)-1H-pyrazole-4-carboxylic acid (0.500 g, 1.66 mmol), 1-hydroxypyrrolidine-2,5-dione (0.190 g, 1.66 mmol), and EDCI (0.380 g, 1.99 mmol) in dichloromethane (10 mL) was stirred at room temperature overnight. The reaction mixture was poured into satd. aqueous NaHCO₃ solution (20 mL), extracted with ethyl acetate (40 mL), washed with brine (10 mL), dried over sodium sulfate, and concentrated to give a residue which was purified by silica gel flash column chromatography using a 33% ethyl acetate in hexane gradient to afford 6-((1-(2-(tert-butoxycarbonyl)benzyl)-1H-pyrazole-4-carbonyl)oxy)-2-(1-(trifluoromethyl)cyclopropane-1-carbonyl)-2,6-diazaspiro[3.5]nonane-9-carboxylic acid (0.655 g, yield 99%) as a colorless oil. MS: [MH]⁺400.0

(S)-6-(1-(2-(tert-butoxycarbonyl)benzyl)-1H-pyrazole-4-carbonyl)-2-(1-(trifluoromethyl)cyclopropanecarbonyl)-2,6-diazaspiro[3.4]octane-8-carboxylic acid: A mixture of 6-((1-(2-(tert-butoxycarbonyl)benzyl)-1H-pyrazole-4-carbonyl)oxy)-2-(1-(trifluoromethyl)cyclopropane-1-carbonyl)-2,6-diazaspiro[3.5]nonane-9-carboxylic acid (0.460 g, 1.15 mmol) and (S)-2-(1-(trifluoromethyl)cyclopropane-1-carbonyl)-2,6-diazaspiro[3.4]octane-8-carboxylic acid (0.320 mg, 1.15 mmol) in tetrahydrofuran (4 mL) and H₂O (4 mL) was stirred at room temperature 4 hours. The reaction mixture was poured into water (20 mL), extracted with ethyl acetate (50 mL), washed with brine (10 mL), dried over sodium sulfate, and concentrated to give a residue which was purified by silica gel flash column chromatography using a 50% ethyl acetate in hexane gradient to afford (S)-6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylic acid (0.160 g, yield 22%) as a white solid. MS: [MH]⁺ 577.0

Tert-butyl 4-((2R,3R)-3-(2-oxabicyclo[2.2.2]octan-4-ylmethoxy)-2-(((benzyloxy)carbonyl)amino)butoxy)piperidine-1-carboxylate: To a solution of tert-butyl 4-hydroxypiperidine-1-carboxylate (0.440 g, 2.19 mmol) and (R)-benzyl 2-((R)-1-(2-oxabicyclo[2.2.2]octan-4-ylmethoxy)ethyl)aziridine-1-carboxylate (0.756 g, 2.19 mmol) in dichloromethane (25 mL) at 0° C. was added boron trifluoride diethyl etherate (0.217 g, 1.53 mmol). The resulting mixture was stirred at this temperature for 2 hours. The reaction mixture was concentrated under reduced pressure to give crude benzyl ((2R,3R)-3-(2-oxabicyclo[2.2.2]octan-4-ylmethoxy)-1-(piperidin-4-yloxy)butan-2-yl)carbamate (crude) as yellow oil which was taken up in THF (8 mL)-water (8 mL), followed by addition of Na₂CO₃ (0.753 g, 8.75 mmol) and Boc₂O (0.716 g, 3.28 mmol). The resulting mixture was stirred at room temperature overnight. The reaction mixture was then poured into water (20 mL) and extracted with ethyl acetate (20 mL×2). The combined organic phases were washed with brine (20 mL×2), dried over anhydrous sodium sulfate, and concentrated under reduced pressure to give a crude residue which was purified by column chromatography using a 20-30% ethyl acetate in hexane gradient to afford tert-butyl 4-((2R,3R)-3-(2-oxabicyclo[2.2.2]octan-4-ylmethoxy)-2-(((benzyloxy)carbonyl)amino)butoxy)piperidine-1-carboxylate (0.180 g, 15% two steps) as colorless oil. MS: [MH]⁺547.35.

Tert-butyl 4-((2R,3R)-3-(2-oxabicyclo[2.2.2]octan-4-ylmethoxy)-2-aminobutoxy)piperidine-1-carboxylate: A mixture of tert-butyl 4-((2R,3R)-3-(2-oxabicyclo[2.2.2]octan-4-ylmethoxy)-2-(((benzyloxy)carbonyl)amino)butoxy)piperidine-1-carboxylate (0.180 g, 0.30 mmol) and palladium on carbon (10%, 0.020 g) in methanol (20 mL) was stirred at room temperature under hydrogen atmosphere (hydrogen balloon) for 3 hours. Palladium on carbon was removed through filtration and washed with methanol (10 mL×2). The combined filtrates were concentrated under reduced pressure to afford tert-butyl 4-((2R,3R)-3-(2-oxabicyclo[2.2.2]octan-4-ylmethoxy)-2-aminobutoxy)piperidine-1-carboxylate (0.100 mg, 74%) as colorless oil. MS: [MH]⁺ 413.40.

Tert-butyl 4-((2R,3R)-3-(2-oxabicyclo[2.2.2]octan-4-ylmethoxy)-2-((S)-6-(1-(2-(tert-butoxycarbonyl)benzyl)-1H-pyrazole-4-carbonyl)-2-(1-(trifluoromethyl)cyclopropanecarbonyl)-2,6-diazaspiro[3.4]octane-8-carboxamido)butoxy)piperidine-1-carboxylate: To a solution of tert-butyl 4-((2R,3R)-3-(2-oxabicyclo[2.2.2]octan-4-ylmethoxy)-2-aminobutoxy)piperidine-1-carboxylate (0.090 g, 0.22 mmol) in N,N-dimethylformamide (4 mL) was added (S)-6-(1-(2-(tert-butoxycarbonyl)benzyl)-1H-pyrazole-4-carbonyl)-2-(1-(trifluoromethyl)cyclopropanecarbonyl)-2,6-diazaspiro[3.4]octane-8-carboxylic acid (0.120 g, 0.22 mmol), N-ethyl-N-isopropylpropan-2-amine (0.084 g, 0.65 mmol), and (2-(7-Aza-1H-benzotriazole-1-yl)-1,1,3,3-tetramethyluronium hexafluorophosphate) (0.100 g, 0.26 mmol). The resulting mixture was stirred at room temperature for 1 hour. The reaction mixture was then poured into water (10 mL) and extracted with ethyl acetate (20 mL×2). The combined organic phases were washed with brine (10 mL×2), dried over anhydrous sodium sulfate, and concentrated under reduced pressure to give a crude residue which was purified by column chromatography using 3-5% methanol in dichloromethane gradient to afford tert-butyl 4-((2R,3R)-3-(2-oxabicyclo[2.2.2]octan-4-ylmethoxy)-2-((S)-6-(1-(2-(tert-butoxycarbonyl)benzyl)-1H-pyrazole-4-carbonyl)-2-(1-(trifluoromethyl)cyclopropanecarbonyl)-2,6-diazaspiro[3.4]octane-8-carboxamido)butoxy)piperidine-1-carboxylate (0.100 g, 66%) as a white solid. MS: [MH]⁺ 971.35.

2-((4-((S)-8-(((2R,3R)-3-(2-oxabicyclo[2.2.2]octan-4-ylmethoxy)-1-(piperidin-4-yloxy)butan-2-yl)carbamoyl)-2-(1-(trifluoromethyl)cyclopropanecarbonyl)-2,6-diazaspiro[3.4]octane-6-carbonyl)-1H-pyrazol-1-yl)methyl)benzoic acid (I-102): A mixture of tert-butyl 4-((2R,3R)-3-(2-oxabicyclo[2.2.2]octan-4-ylmethoxy)-2-((S)-6-(1-(2-(tert-butoxycarbonyl)benzyl)-1H-pyrazole-4-carbonyl)-2-(1-(trifluoromethyl)cyclopropanecarbonyl)-2,6-diazaspiro[3.4]octane-8-carboxamido)butoxy)piperidine-1-carboxylate (0.080 g, 0.08 mmol) and 2,2,2-trifluoroacetic acid (6 ml) in anhydrous dichloromethane (6 ml) was stirred at room temperature for 2 hours. TLC showed the reaction was complete. The volatiles were evaporated under reduced pressure to give a crude residue which was purified by prep-HPLC to afford 2-((4-((S)-8-(((2R,3R)-3-(2-oxabicyclo[2.2.2]octan-4-ylmethoxy)-1-(piperidin-4-yloxy)butan-2-yl)carbamoyl)-2-(1-(trifluoromethyl)cyclopropanecarbonyl)-2,6-diazaspiro[3.4]octane-6-carbonyl)-1H-pyrazol-1-yl)methyl)benzoic acid (37.8 mg, 56%) as a white solid. ¹HNMR (400 MHz, CD₃OD): δ 8.27-8.22 (m, 1H), 8.09-8.07 (m, 1H), 7.97-7.93 (m, 1H), 7.54 (t, J=7.6 Hz, 1H), 7.46 (t, J=7.6 Hz, 1H), 7.03 (d, J=7.6 Hz, 1H), 5.85-5.84 (m, 2H), 4.57-3.87 (m, 9H), 3.77-3.55 (m, 8H), 3.39-3.34 (m, 1H), 3.30-3.25 (m, 4H), 3.12-3.08 (m, 2H), 3.01-2.97 (m, 1H), 2.01-1.98 (m, 4H), 1.89-1.66 (m, 7H), 1.57-1.50 (m, 3H), 1.11-1.08 (m, 3H). MS: [MH]⁺ 815.65.

### Synthesis of (S)—N-((2R,3R)-3-(2-oxabicyclo[2.2.2]octan-4-ylmethoxy)-1-((4-(thiazol-2-yl)cyclohexyl)oxy)butan-2-yl)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamide (I-107)

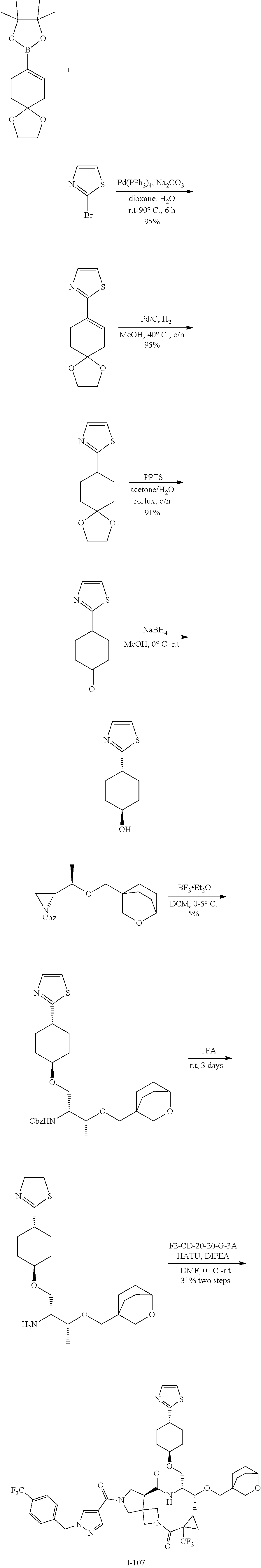

2-(1,4-Dioxaspiro[4.5]dec-7-en-8-yl)thiazole: A mixture of 2-bromothiazole (1.000 g, 6.10 mmol), 4,4,5,5-tetramethyl-2-(1,4-dioxaspiro[4.5]dec-7-en-8-yl)-1,3,2-dioxaborolane (1.947 g, 7.32 mmol), tetrakis(triphenylphosphine)palladium (0.352 g, 0.30 mmol), and sodium carbonate (3.231 g, 30.48 mmol) in 1,4-dioxane (30 mL) and water (15 mL) was stirred at 90° C. under nitrogen atmosphere for 6 hours. The reaction mixture was poured into water (10 mL) and extracted with ethyl acetate (30 mL×2). The combined organic layers were washed with brine (30 mL), dried over anhydrous sodium sulfate, and concentrated to give a crude residue which was purified by silica gel flash column chromatography using a 12.5% ethyl acetate in hexane gradient to afford 2-(1,4-dioxaspiro[4.5]dec-7-en-8-yl)thiazole (1.300 g, 95%) as a yellow oil. MS: [MH]⁺224.20.

2-(1,4-Dioxaspiro[4.5]decan-8-yl)thiazole: To a solution of 2-(1,4-dioxaspiro[4.5]dec-7-en-8-yl)thiazole (1.300 g, 5.82 mmol) in ethyl acetate (70 mL) was added palladium (10%, 0.650 g), and the mixture was stirred at 40° C. under hydrogen overnight. The reaction mixture was filtered, and the filtrate was concentrated to give a crude residue which was purified by silica gel flash column chromatography using a hexane-ethyl acetate-dichloromethane (3:1:1, v/v/v) gradient to afford 2-(1,4-dioxaspiro[4.5]decan-8-yl)thiazole (1.260 g, 95%) as a colorless oil. MS: [MH]⁺226.20

4-(Thiazol-2-yl)cyclohexanone: A solution of 2-(1,4-dioxaspiro[4.5]decan-8-yl)thiazole (0.570 g, 2.530 mmol) and pyridinium p-toluenesulfonate (1.272 g, 5.060 mmol) in a mixture of acetone (12 mL) and water (12 mL) was refluxed overnight. The reaction mixture was poured into water (10 mL) and extracted with ethyl acetate (30 mL×2). The combined organic layers were washed with brine (30 mL), dried over anhydrous sodium sulfate and concentrated to afford 4-(thiazol-2-yl)cyclohexanone (0.430 g, 91%) as a white solid. MS: [MH]⁺182.30.

Trans-4-(thiazol-2-yl)cyclohexanol: To a solution of 4-(thiazol-2-yl)cyclohexanone (1.500 g, 8.276 mmol) in methanol was added sodium borohydride (0.193 g, 16.552 mmol) at 0° C. and the mixture was stirred at room temperature under nitrogen atmosphere for 1.5 hours. The reaction mixture was poured into water (10 mL) and extracted with ethyl acetate (30 mL×2). The combined organic layers were washed with brine (30 mL), dried over anhydrous sodium sulfate, and concentrated to give a crude residue which was purified by silica gel flash column chromatography using a hexane-ethyl acetate-dichloromethane (1:1:1, v/v/v) gradient to afford trans-4-(thiazol-2-yl)cyclohexanol (0.580 g) as a colorless oil MS: [MH]⁺184.10 ¹H NMR (400 MHz, CDCl₃) δ 7.69 (d, J=1.8 Hz, 1H), 7.20 (d, J=1.6 Hz, 1H), 3.75-3.67 (m, 1H), 3.03-2.96 (m, 1H), 2.24-2.21 (m, 2H), 2.24-2.10 (m, 2H), 1.71-1.64 (m, 2H), 1.50-1.39 (m, 2H).

Benzyl ((2R,3R)-3-(2-oxabicyclo[2.2.2]octan-4-ylmethoxy)-1-((4-(thiazol-2-yl)cyclohexyl)oxy)butan-2-yl)carbamate: To a solution of (R)-benzyl 2-((R)-1-(2-oxabicyclo[2.2.2]octan-4-ylmethoxy)ethyl)aziridine-1-carboxylate (0.510 g, 1.650 mmol) and trans-4-(thiazol-2-yl)cyclohexanol (0.605 g, 3.300 mmol) in dichloromethane (6 mL) at 0° C. was added boron trifluoride etherate (0.5 mL) slowly, and the mixture was stirred at 0-5° C. under nitrogen atmosphere for two hours. The reaction mixture was concentrated to give a crude residue which was purified by silica gel flash column chromatography using a 50% ethyl acetate in hexane gradient to afford benzyl ((2R,3R)-3-(2-oxabicyclo[2.2.2]octan-4-ylmethoxy)-1-((4-(thiazol-2-yl)cyclohexyl)oxy)butan-2-yl)carbamate (0.035 g, 5% yield) as a colorless oil. MS: [MH]⁺ 529.25.

(S)—N-((2R,3R)-3-(2-oxabicyclo[2.2.2]octan-4-ylmethoxy)-1-((4-(thiazol-2-yl)cyclohexyl)oxy)butan-2-yl)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamide (I-107): A mixture of benzyl ((2R,3R)-3-(2-oxabicyclo[2.2.2]octan-4-ylmethoxy)-1-((4-(thiazol-2-yl)cyclohexyl)oxy)butan-2-yl)carbamate (0.030 g, 0.057 mmol) in trifluoroacetic acid (8.5 mL) was stirred at room temperature for 80 hours. The reaction mixture was concentrated under reduced pressure to afford (2R,3R)-3-(2-oxabicyclo[2.2.2]octan-4-ylmethoxy)-1-((4-(thiazol-2-yl)cyclohexyl)oxy)butan-2-amine (crude) as a brown oil which was taken up in N,N-dimethylformamide (1 mL), followed by the addition of (S)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxylic acid (0.031 g, 0.057 mmol), N-ethyl-N-isopropylpropan-2-amine (0.037 g, 0.285 mmol), and (2-(7-Aza-1H-benzotriazole-1-yl)-1,1,3,3-tetramethyluronium hexafluorophosphate) (0.026 g, 0.068 mmol) at 0-5° C. The resulting mixture was stirred at room temperature under nitrogen for two hours. The reaction mixture was poured into saturated aqueous ammonium chloride solution (15 mL) and extracted with ethyl acetate (10 mL). The organic layer was washed with water (8 mL×3) and brine (8 mL), dried over anhydrous sodium sulfate, and concentrated under reduced pressure to give a crude residue which was purified by prep-TLC using a 7% methanol in dichloromethane gradient to afford (S)—N-((2R,3R)-3-(2-oxabicyclo[2.2.2]octan-4-ylmethoxy)-1-((4-(thiazol-2-yl)cyclohexyl)oxy)butan-2-yl)-6-(1-(4-(trifluoromethyl)benzyl)-1H-pyrazole-4-carbonyl)-2-(1-(trifluoromethyl)cyclopropanecarbonyl)-2,6-diazaspiro[3.4]octane-8-carboxamide (0.019 g, two steps yields 31%) as a white solid. ¹H NMR (400 MHz, CD₃OD) δ 8.28 (d, J=7.4 Hz, 1H), 7.94 (d, J=6.6 Hz, 1H), 7.91-7.85 (m, 1H), 7.68-7.63 (m, 3H), 7.44-7.43 (m, 3H), 5.48-5.47 (m, 2H), 4.58-4.21 (m, 3H), 4.11-3.81 (m, 6H), 3.76 (s, 3H), 3.65-3.58 (m, 2H), 3.54-3.47 (m, 1H), 3.36-3.35 (m, 1H), 3.27-3.24 (m, 2H), 3.02-2.98 (m, 2H), 2.17-1.97 (m, 6H), 1.73-1.53 (m, 8H), 1.42-1.36 (m, 1H), 1.24-1.23 (m, 5H), 1.08-1.04 (m, 3H). MS: [MH]⁺921.60.

### Synthesis of (S)—N-((2S,3R)-1-oxo-1-(4-(thiazol-2-yl)piperidin-1-yl)-3-((1-(trifluoromethyl)cyclopropyl)methoxy)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide I-80

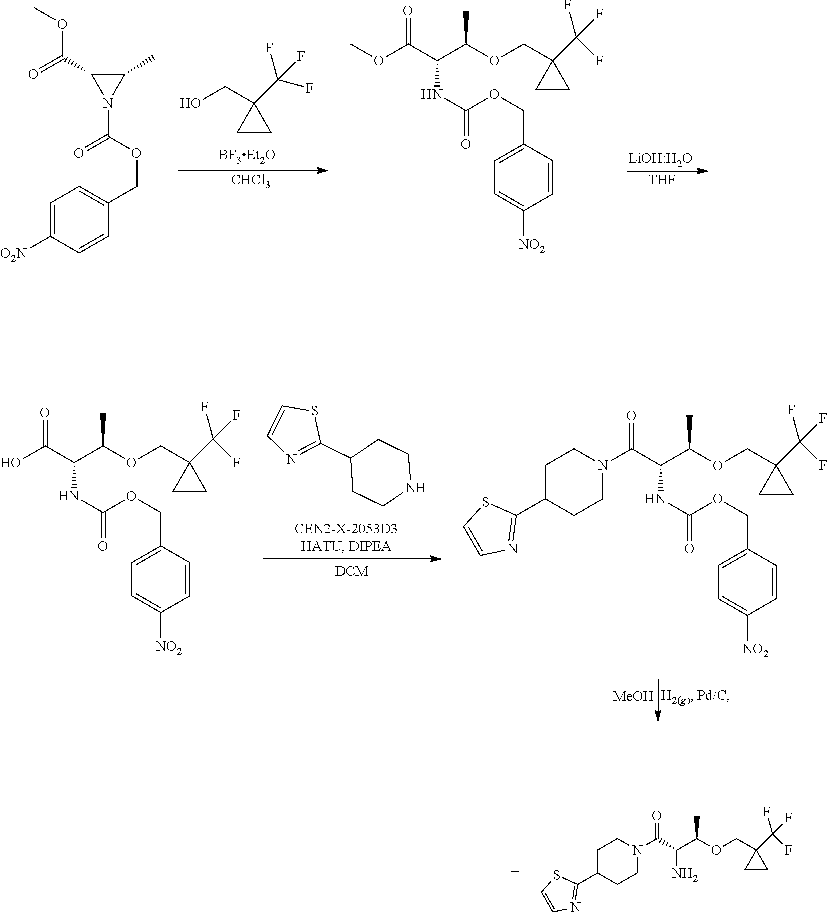

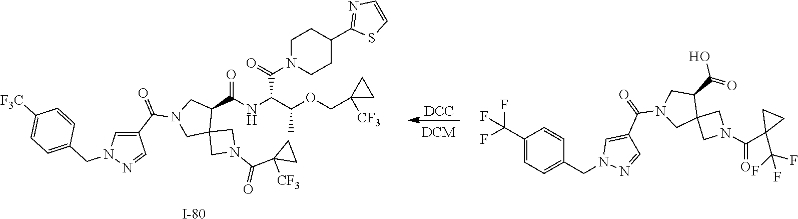

methyl N-(((4-nitrobenzyl)oxy)carbonyl)-O-((1-(trifluoromethyl)cyclopropyl)methyl)-L-threoninate To a stirred solution of 2-methyl 1-(4-nitrobenzyl) (2S,3S)-3-methylaziridine-1,2-dicarboxylate (0.300 g, 1.02 mmol) in CHCl₃ (3 mL) was added (1-(trifluoromethyl)cyclopropyl)methanol (0.285 g, 2.04 mmol) at 0° C. under nitrogen. After 15 minutes of stirring at the same temperature, was added boron trifluoride etherate (0.070 g, 0.51 mmol) and stirring was continued for 1 h at room temperature. [Two more batches of 1 g and 2 g were performed and worked up together]. Reaction mixture was concentrated under reduced pressure to get a crude product, which was purified by silica gel column chromatography, using ethyl acetate-hexane=3:7→2:3 to afford methyl N-(((4-nitrobenzyl)oxy)carbonyl)-O-((1-(trifluoromethyl)cyclopropyl)methyl)-L-threoninate (3.9 g, 80%) as an off-white solid. MS: [MH]⁺ 435.22.

N-(((4-nitrobenzyl)oxy)carbonyl)-O-((1-(trifluoromethyl)cyclopropyl)methyl)-L-threonine To a stirred solution of methyl N-(((4-nitrobenzyl)oxy)carbonyl)-O-((1-(trifluoromethyl)cyclopropyl)methyl)-L-threoninate (1.6 g, 3.68 mmol) in THF (15 mL), was added LiOH·H₂O (0.620 g, 14.74 mmol) at room temperature and the resulting reaction mixture was stirred at same temperature for 12 h. [Another identical batch of 2.3 g was performed and worked up together]. Reaction mixture was back-washed with diethylether (150 mL×2), collected aqueous solution and acidified (pH~2-3) with an aqueous 1N HCl solution (200 mL) and was extracted with EtOAc (250 mL×2). Combined organic extracts dried over anhydrous Na₂SO₄ and concentrated under reduced pressure to afforded N-(((4-nitrobenzyl)oxy)carbonyl)-O-((1-(trifluoromethyl)cyclopropyl)methyl)-L-threonine [3.1 g, 82% (crude)] as a yellow liquid. MS: [MH]⁺421.24.

N-(((4-nitrobenzyl)oxy)carbonyl)-O-((1-(trifluoromethyl)cyclopropyl)methyl)-L-threonine To a stirred solution of methyl N-(((4-nitrobenzyl)oxy)carbonyl)-O-((1-(trifluoromethyl)cyclopropyl)methyl)-L-threoninate (1.6 g, 3.68 mmol) in THF (15 mL), was added LiOH·H₂O (0.620 g, 14.74 mmol) at room temperature and the resulting reaction mixture was stirred at same temperature for 12 h. [Another identical batch of 2.3 g was performed and worked up together]. Reaction mixture was back-washed with diethylether (150 mL×2), collected aqueous solution and acidified (pH~2-3) with an aqueous 1N HCl solution (200 mL) and was extracted with EtOAc (250 mL×2). Combined organic extracts dried over anhydrous Na₂SO₄ and concentrated under reduced pressure to afforded N-(((4-nitrobenzyl)oxy)carbonyl)-O-((1-(trifluoromethyl)cyclopropyl)methyl)-L-threonine [3.1 g, 82% (crude)] as a yellow liquid. MS: [MH]⁺421.24.

### Synthesis of 2-(piperidin-4-yl)thiazole was mentioned under (S)—N-((2S,3R)-1-oxo-3-((tetrahydro-2H-pyran-4-yl)methoxy)-1-(4-(thiazol-2-yl)piperidin-1-yl)butan-2-yl)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamide

4-nitrobenzyl ((2S,3R)-1-oxo-1-(4-(thiazol-2-yl)piperidin-1-yl)-3-((1-(trifluoromethyl)cyclopropyl)methoxy)butan-2-yl)carbamate: To a stirred solution of N-(((4-nitrobenzyl)oxy)carbonyl)-O-((1-(trifluoromethyl)cyclopropyl)methyl)-L-threonine (1.0 g, 2.38 mmol) in DCM (10 mL) was added DIPEA (0.920 g, 7.14 mmol) at 0° C. temperature under nitrogen. After 15 minutes of stirring at the same temperature, was added and 2-(piperidin-4-yl)thiazole (0.600 g, 3.57 mmol) followed by HATU (1.8 g, 4.76 mmol) and stirring was continued for an additional 1 h at room temperature. Reaction mixture was dilute with water (300 mL) and was extracted with DCM (200 mL×3). Combined organic extracts dried over anhydrous Na₂SO₄ and concentrated under reduced pressure to get a crude product. Resulting crude was purified by silica gel column chromatography, using MeOH-DCM=0.5:9.5→1:9 as a gradient, to afford 4-nitrobenzyl ((2S,3R)-1-oxo-1-(4-(thiazol-2-yl)piperidin-1-yl)-3-((1-(trifluoromethyl)cyclopropyl)methoxy)butan-2-yl)carbamate (1.2 g, 88%) as a yellow oil. MS: [MH]⁺ 571.26.

(2S,3R)-2-amino-1-(4-(thiazol-2-yl)piperidin-1-yl)-3-((1-(trifluoromethyl)cyclopropyl)methoxy) butan-1-one To a stirred solution of 4-nitrobenzyl ((2S,3R)-1-oxo-1-(4-(thiazol-2-yl)piperidin-1-yl)-3-((1-(trifluoromethyl)cyclopropyl)methoxy)butan-2-yl)carbamate (1.1 g, 1.92 mmol) in MeOH (10 mL) was added 10% Pd on activated carbon (0.500 g) at room temperature under nitrogen and the resulting mixture was hydrogenated under balloon pressure at the same temperature for 2 h. Reaction mixture was filter through a celite bed, washed the bed with methanol (500 mL) and collected filtrates were concentrated under reduced pressure to afford (2S,3R)-2-amino-1-(4-(thiazol-2-yl)piperidin-1-yl)-3-((1-(trifluoromethyl)cyclopropyl)methoxy)butan-1-one (0.450 g, 55%) as a yellow oil. MS: [MH]⁺ 392.29.

### Synthesis of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid was mentioned under 4-(1-(O-((2-Oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid

(S)—N-((2S,3R)-1-oxo-1-(4-(thiazol-2-yl)piperidin-1-yl)-3-((1-(trifluoromethyl)cyclopropyl)methoxy)butan-2-yl)-6-(1-(4-(trifluoromethyl)benzyl)-H-pyrazole-4-carbonyl)-2-(1-(trifluoromethyl)cyclopropane-1-carbonyl)-2,6-diazaspiro[3.4]octane-8-carboxamide I-80 To a stirred solution of (2S,3R)-2-amino-1-(4-(thiazol-2-yl)piperidin-1-yl)-3-((1-(trifluoromethyl)cyclopropyl)methoxy)butan-1-one (0.286 g, 0.73 mmol) in DCM (5 mL) was added (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (0.290 g, 0.36 mmol) at room temperature under nitrogen. After 15 minutes of stirring at the same temperature, was added DCC (0.151 g, 0.73 mmol) into the reaction mixture and the resulting reaction mixture stirred for 1 h at the same temperature. The reaction mixture was dilute with water (100 mL) and was extracted with DCM (100 mL×3). Combined organic extracts dried over anhydrous Na₂SO₄ and concentrated under reduced pressure to get a crude product, which was purified by silica gel column chromatography, using MeOH-DCM=0.3:9.7→40.6:9.4 as a gradient, to afford (S)—N-((2S,3R)-1-oxo-1-(4-(thiazol-2-yl)piperidin-1-yl)-3-((1-(trifluoromethyl)cyclopropyl)methoxy)butan-2-yl)-6-(1-(4 (trifluoromethyl)benzyl)-1H-pyrazole-4-carbonyl)-2-(1-(trifluoromethyl)cyclopropane-1-carbonyl)-2,6-diazaspiro[3.4]octane-8-carboxamide (1.2 g, 88%) as an off white solid. ¹H-NMR: (400 MHz, DMSO-d₆) δ 8.49-8.39 (m, 2H), 7.88-7.82 (m, 1H), 7.73-7.70 (m, 3H), 7.62-7.56 (m, 1H), 7.45-7.43 (m, 2H), 5.45 (s, 2H), 4.95 (brs, 1H), 4.41 (brs, 2H), 4.18-3.87 (m, 6H), 3.76-3.50 (m, 6H), 3.24-3.16 (m, 1H; peak merged with moisture from DMSO-d₆) 2.82-2.67 (m, 1H), 2.06-1.99 (m, 2H), 1.71-1.69 (m, 1H), 1.57-1.49 (m, 2H), 1.23-1.11 (m, 4H), 1.07-1.06 (m, 3H), 0.92-0.87 (m, 4H). MS: [MH]⁺918.52

### Synthesis of 4-(1-(N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-O-((1-(trifluoromethyl)cyclopropyl)methyl)-L-threonyl)piperidin-4-yl)benzoic acid (I-68)

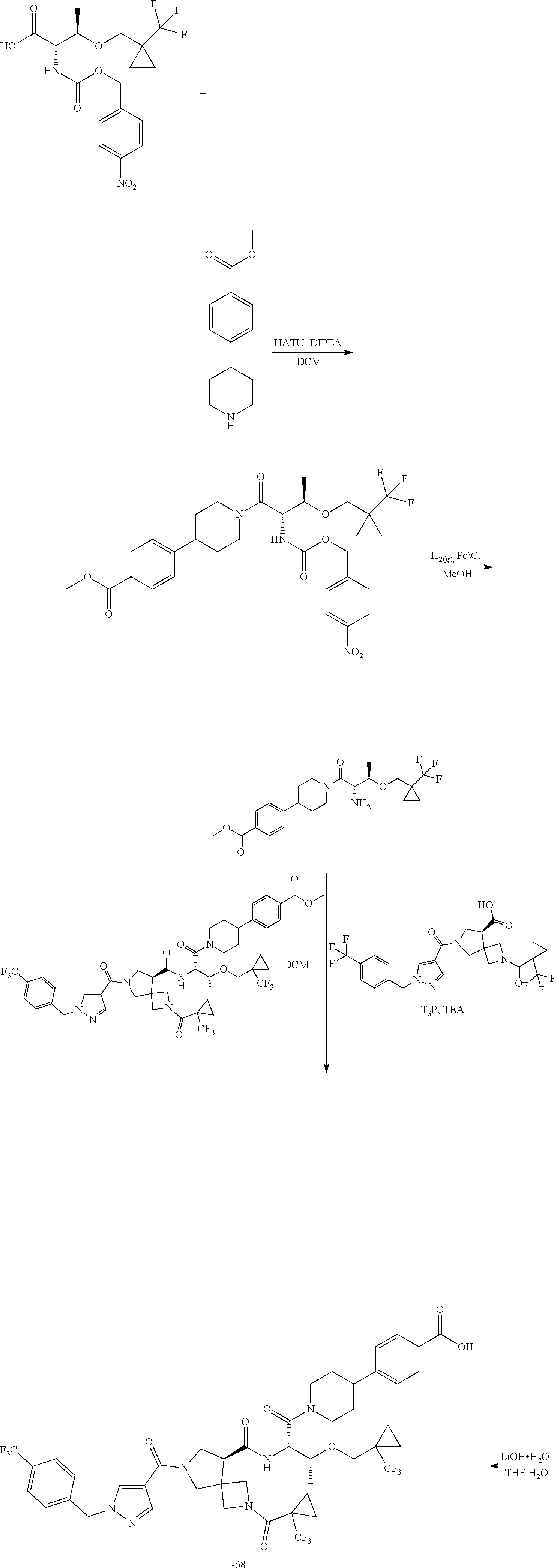

### Synthesis of methyl 4-(piperidin-4-yl)benzoate was mentioned under 4-(1-(O-((tetrahydro-2H-pyran-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)benzoic acid synthesis

Methyl 4-(1-(N-(((4-nitrobenzyl)oxy)carbonyl)-O-((1-(trifluoromethyl)cyclopropyl)methyl)-L-threonyl)piperidin-4-yl)benzoate To a stirred solution of N-(((4-nitrobenzyl)oxy)carbonyl)-O-((1-(trifluoromethyl)cyclopropyl)methyl)-L-threonine (1.20 g, 2.85 mmol) in DCM (10 mL), was added DIPEA (1.10 g, 8.56 mmol) and HATU (2.17 g, 5.71 mmol) at 0° C. under nitrogen. After 10 minutes of stirring at the same temperature, was added methyl 4-(piperidin-4-yl)benzoate (0.930 g, 4.28 mmol) and stirring was continued for 1 h at room temperature. Reaction mixture was slowly poured into ice water (50 mL) and was extracted with DCM (50 mL×3). Organic extracts washed with brine (50 mL), dried over anhydrous Na₂SO₄ and concentrated under reduced pressure to get a crude product. The crude product was purified by silica gel column chromatography, using ethyl acetate-hexane=0:142:3 as a gradient, to afford methyl 4-(1-(N-(((4-nitrobenzyl)oxy)carbonyl)-O-((1-(trifluoromethyl)cyclopropyl)methyl)-L-threonyl)piperidin-4-yl)benzoate (1.60 g, 90%) as a colorless oil. MS: [MH]⁺ 622.4.

Methyl 4-(1-(O-((1-(trifluoromethyl)cyclopropyl)methyl)-L-threonyl)piperidin-4-yl)benzoate To a stirred solution of Methyl 4-(1-(N-(((4-nitrobenzyl)oxy)carbonyl)-O-((1-(trifluoromethyl)cyclopropyl)methyl)-L-threonyl)piperidin-4-yl)benzoate (1.5 g, 2.41 mmol) in methanol (15 mL), was added 10% Pd on activated carbon (0.700 g) at room temperature and the resulting reaction mixture was hydrogenated under balloon pressure for 1 h at the same temperature. Reaction mixture was filtered through celite bed, washed the bed with methanol (100 mL) and collected filtrates were concentrated under reduced pressure to get a crude product. The crude product was purified by silica gel column chromatography, using MeOH-DCM=0:1→1:9 as an eluent, to afford methyl 4-(1-(O-((1-(trifluoromethyl)cyclopropyl)methyl)-L-threonyl)piperidin-4-yl)benzoate (1.0 g, 94%) as a colorless oil. MS: [MH]⁺ 443.3.

### Synthesis of (S)-6-(1-(4-(Trifluoromethyl)benzyl)-1H-pyrazole-4-carbonyl)-2-(1(trifluoromethyl)cyclopropane-1-carbonyl)-2,6-diazaspiro[3.4]octane-8-carboxylic acid mentioned under 4-(1-(O-((2-Oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid

Methyl 4-(1-(N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-O-((1-(trifluoromethyl)cyclopropyl)methyl)-L-threonyl)piperidin-4-yl)benzoate To a stirred solution of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (0.300 g, 0.55 mmol) in DCM (5 mL) were added methyl 4-(1-(O-((1-(trifluoromethyl)cyclopropyl)methyl)-L-threonyl)piperidin-4-yl)benzoate (0.24 g, 0.55 mmol), triethylamine (0.166 g, 1.65 mmol) and T₃P (50% solution in ethylacetate) (0.350 g, 1.10 mmol) sequentially at 0° C. under nitrogen and the resulting mixture was stirred for 1 h at room temperature. Reaction mixture was slowly poured into ice water (70 mL) and was extracted with DCM (50 mL×3). Combined organic extracts were wash with brine (20 mL), dried over anhydrous Na₂SO₄ and concentrated under reduced pressure to get a crude product. The crude product was purified by silica gel column chromatography, using MeOH-DCM=0:1→1:9 as an eluent, to afford methyl 4-(1-(N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-O-((1-(trifluoromethyl)cyclopropyl)methyl)-L-threonyl)piperidin-4-yl)benzoate (0.280 g, 52%) as an off white solid. MS: [MH]⁺ 969.5.

4-(1-(N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-O-((1-(trifluoromethyl)cyclopropyl)methyl)-L-threonyl)piperidin-4-yl)benzoic acid I-68 To a stirred solution of Methyl 4-(1-(N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-O-((1-(trifluoromethyl)cyclopropyl)methyl)-L-threonyl)piperidin-4-yl)benzoate (0.260 g, 0.26 mmol) in a mixture of THF-water (3:1; 5 mL), was added lithium hydroxide monohydrate (0.033 g, 0.80 mmol) at room temperature and the resulting reaction mixture was stirred for 5 h at the same temperature. Reaction mixture was concentrated under reduced pressure, obtained crude was diluted with water (40 mL) and it was washed with diethyl ether (60 mL×2) to remove unwanted organic impurities. Aqueous part was acidified (pH ~2-3) with an aqueous solution of 1N HCl and was extracted with ethyl acetate (50 mL×3). Combined organic extracts were wash with brine (100 mL), dried over anhydrous Na₂SO₄ and concentrated under reduced pressure to get a crude product. The crude product was purified by Prep-HPLC using 0.1% formic acid in water-acetonitrile as gradient, to afforded 4-(1-(N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-O-((1-(trifluoromethyl)cyclopropyl)methyl)-L-threonyl)piperidin-4-yl)benzoic acid. (0.050 g, 20%) as a white solid. ¹H-NMR (400 MHz, DMSO-d₆) δ 13.02-12.78 (brs, 1H), 8.43-8.39 (m, 2H), 7.88-7.83 (m, 3H), 7.73-7.70 (m, 2H), 7.44-7.43 (m, 2H), 7.38-7.29 (m, 2H), 5.48 (s, 2H), 5.03-4.85 (m, 1H), 4.61-4.48 (m, 1H), 4.44-4.10 (m, 3H), 3.95-3.80 (m, 3H), 3.80-3.51 (m, 6H), 3.19-3.12 (m, 2H), 3.95-3.80 (m, 1H), 2.67-2.56 (m, 1H), 1.90-1.70 (m, 2H), 1.70-1.30 (m, 2H), 1.25-1.05 (m, 5H), 0.93-0.84 (m, 4H). MS: [MH]⁺ 955.5.

### Synthesis of 1-((2R,3R)-3-((2-oxabicyclo[2.2.2]octan-4-yl)methoxy)-2-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butyl)piperidine-4-carboxylic acid as I-97

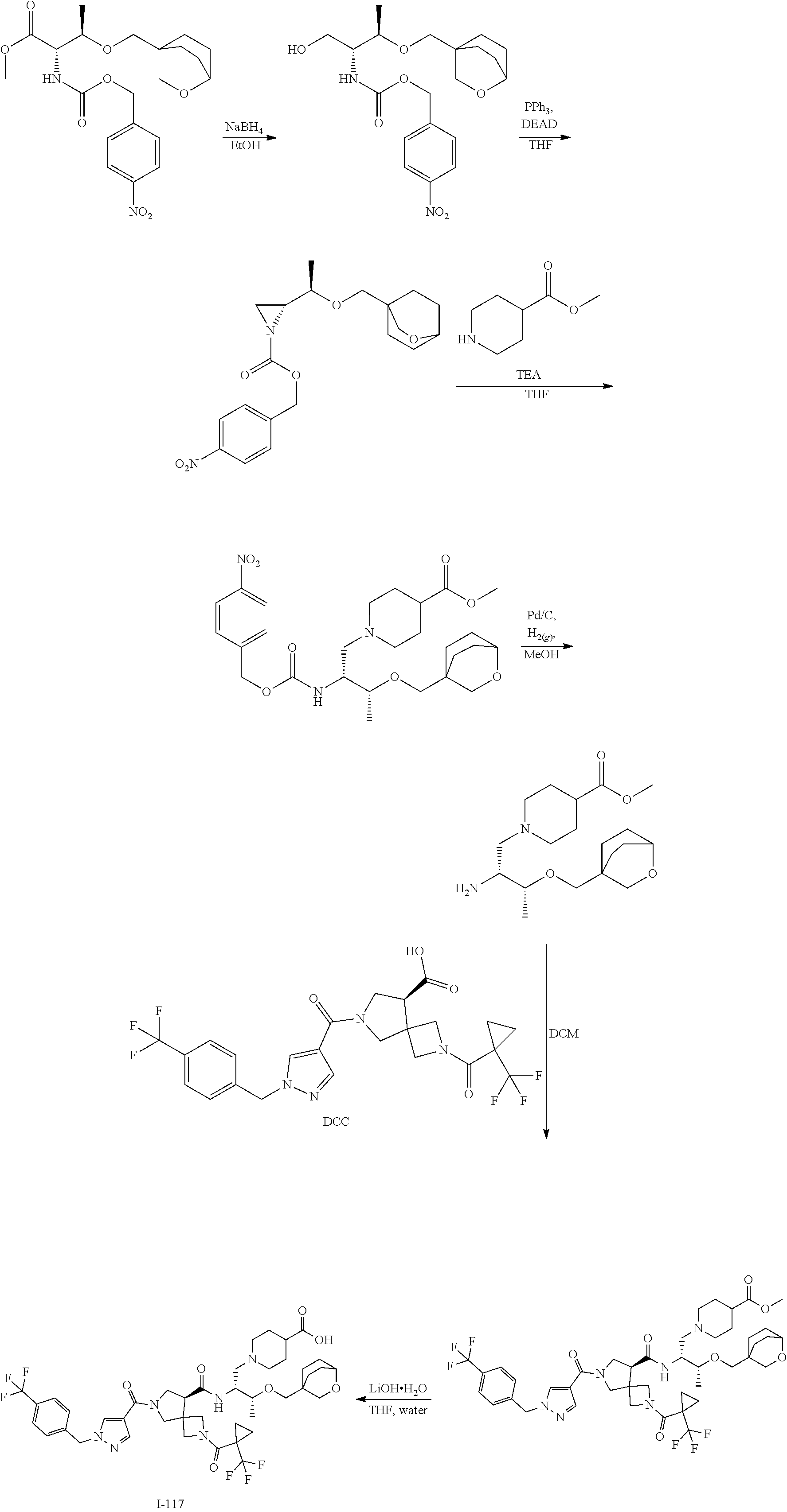

Synthetic procedure of To a stirred solution of methyl O-((2-oxabicyclo[2.2.2]octan-4-yl)methyl)-N-(((4-nitrobenzyl)oxy)carbonyl)-L-threoninate mentioned under Methyl 2-(1-(O-((2-oxabicyclo[2.2.2]octan-4-yl)methyl)-L-threonyl)piperidin-4-yl)-5-(trifluoromethyl)benzoate

4-Nitrobenzyl ((2R,3R)-3-((2-oxabicyclo[2.2.2]octan-4-yl)methoxy)-1-hydroxybutan-2-yl)carbamate To a stirred solution of methyl O-((2-oxabicyclo[2.2.2]octan-4-yl)methyl)-N-(((4-nitrobenzyl)oxy)carbonyl)-L-threoninate (0.500 g, 1.14 mmol) in ethanol (5 mL) was added NaBH₄ (0.130 g, 3.44 mmol) at 0° C. under nitrogen and the resulting mixture was stirred for 3 h at room temperature. [Another five batches of 0.500 g were performed and worked up together]. Reaction mixture was slowly poured into ice water (200 mL) and was extracted with ethyl acetate (100 mL×3). Organic extracts washed with brine (200 mL), dried over anhydrous Na₂SO₄ and concentrated under reduced pressure to get a crude product. The crude product was purified by silica gel column chromatography, using ethyl acetate-hexane=0:1→3:2 as an eluent, to afford 4-nitrobenzyl ((2R,3R)-3-((2-oxabicyclo[2.2.2]octan-4-yl)methoxy)-1-hydroxybutan-2-yl)carbamate (1.50 g, 53%) as an light brown oil. MS: [MH]⁺ 409.3.

4-Nitrobenzyl (R)-2-((R)-1-((2-oxabicyclo[2.2.2]octan-4-yl)methoxy)ethyl)aziridine-1-carboxylate To a stirred solution of 4-nitrobenzyl ((2R,3R)-3-((2-oxabicyclo[2.2.2]octan-4-yl)methoxy)-1-hydroxybutan-2-yl)carbamate (1.50 g, 3.67 mmol) in THF (5 mL) were added PPh₃ (1.4 g, 5.51 mmol) and DEAD (0.959 g, 5.51 mmol) at 0° C. under nitrogen and resulting mixture was stirred for 16 h at room temperature. After completion of reaction, reaction mixture was concentrated under reduced pressure. Resulting crude was purified by silica gel column chromatography, using ethyl acetate-hexane=0:142:1 to afford 4-nitrobenzyl (R)-2-((R)-1-((2-oxabicyclo[2.2.2]octan-4-yl)methoxy)ethyl)aziridine-1-carboxylate (1.0 g, 70%) as an colourless semi solid. MS: [MH]⁺ 391.3

Methyl 1-((2R,3R)-3-((2-oxabicyclo[2.2.2]octan-4-yl)methoxy)-2-((((4-nitrobenzyl)oxy)carbonyl)amino)butyl)piperidine-4-carboxylate To a stirred solution of 4-nitrobenzyl (R)-2-((R)-1-((2-oxabicyclo[2.2.2]octan-4-yl)methoxy)ethyl)aziridine-1-carboxylate (0.500 g, 1.28 mmol) and methyl piperidine-4-carboxylate (0.364 g, 2.56 mmol) in THF (4 mL) was added trimethylamine (0.258 g, 2.56 mmol) at room temperature under nitrogen and the resulting mixture was heated at 80° C. for 16 h. After cooling to room temperature, reaction mixture was slowly poured into water (50 mL) and was extracted with ethyl acetate (50 mL×3). Combined organic extracts were wash with brine (50 mL), dried over anhydrous Na₂SO₄ and concentrated under reduced pressure to get a crude product. Resulting crude was purified by silica gel column chromatography, using ethyl acetate-hexane=0:1→4:1 as a gradient, to afford methyl 1-((2R,3R)-3-((2-oxabicyclo[2.2.2]octan-4-yl)methoxy)-2-((((4-nitrobenzyl)oxy)carbonyl)amino)butyl)piperidine-4-carboxylate (0.250 g, 37%) as an brown sticky solid. MS: [MH]⁺ 534.3.

Methyl 1-((2R,3R)-3-((2-oxabicyclo[2.2.2]octan-4-yl)methoxy)-2-aminobutyl)piperidine-4-carboxylate To a stirred solution of Methyl 1-((2R,3R)-3-((2-oxabicyclo[2.2.2]octan-4-yl)methoxy)-2-((((4-nitrobenzyl)oxy)carbonyl)amino)butyl)piperidine-4-carboxylate (0.250 g, 0.46 mmol) in methanol (5 mL) was added 10% Pd on activated carbon (0.120 g) at room temperature under nitrogen and the resulting mixture was hydrogenated under balloon pressure for 2 h at the same temperature. Reaction mixture was filter through a celite bed, washed the bed with methanol (50 mL) and collected filtrates were concentrated under reduced pressure. Resulting crude was purified by silica gel column chromatography, using methanol-DCM=0:141:9 as gradient, to afford methyl 1-((2R,3R)-3-((2-oxabicyclo[2.2.2]octan-4-yl)methoxy)-2-aminobutyl)piperidine-4-carboxylate (0.140 g, 84%) as a colorless sticky solid. MS: [MH]⁺ 355.4

### Synthetic procedure of (S)-6-(1-(4-(trifluoromethyl)benzyl)-JH-pyrazole-4-carbonyl)-2-(1-(trifluoromethyl)cyclopropane-1-carbonyl)-2,6-diazaspiro[3.4]octane-8-carboxylic acid mentioned under 4-(1-(O-((2-Oxabicyclo[2.2.2]octan-4-yl)methyl)-N—((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)piperidin-4-yl)-2-hydroxybenzoic acid

Methyl 1-((2R,3R)-3-((2-oxabicyclo[2.2.2]octan-4-yl)methoxy)-2-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butyl)piperidine-4-carboxylate To a stirred solution of (S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid] (0.168 g, 0.308 mmol) in DCM (3 mL) were added methyl 1-((2R,3R)-3-((2-oxabicyclo[2.2.2]octan-4-yl)methoxy)-2-aminobutyl)piperidine-4-carboxylate (0.110 g, 0.31 mmol) and DCC (0.128 g, 0.62 mmol) sequentially at 0° C. under nitrogen and the resulting reaction mixture stirred for 30 minutes at room temperature. Reaction mixture was concentrated under reduced pressure and the resulting crude product was purified by silica gel column chromatography, using methanol-DCM=0:1→1:9 as a gradient, to afford methyl 1-((2R,3R)-3-((2-oxabicyclo[2.2.2]octan-4-yl)methoxy)-2-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butyl)piperidine-4-carboxylate (0.100 g, 37%) as an off white solid. MS: [MH]⁺ 881.7

1-((2R,3R)-3-((2-oxabicyclo[2.2.2]octan-4-yl)methoxy)-2-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butyl)piperidine-4-carboxylic acid I-7 To a stirred solution of methyl 1-((2R,3R)-3-((2-oxabicyclo[2.2.2]octan-4-yl)methoxy)-2-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butyl)piperidine-4-carboxylate (0.100 g, 0.113 mmol) in a mixture of THF-water (3:1; 3 mL) was added lithium hydroxide monohydrate (0.014 g, 0.340 mmol) at room temperature and the mixture was heated at 65° C. for 2 h. [Another identical hatch of 0.100 g was performed and worked up together]. The reaction mixture was concentrated under reduced pressure, obtained crude was diluted with water (20 mL) and was washed with diethyl ether (20 mL×2) to remove unwanted organic impurities. Aqueous part was acidified (pH ~2-3) with an aqueous solution of 1N HCl and was extracted with ethyl acetate (30 mL×3). Combined organic extracts were wash with brine (30 mL), dried over anhydrous Na₂SO₄ and concentrated under reduced pressure to get a crude product. The crude product was purified by prep-HPLC, 0.1% formic acid in water and acetonitrile as gradient, to afford 1-((2R,3R)-3-((2-oxabicyclo[2.2.2]octan-4-yl)methoxy)-2-((S)-6-(1-(4-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butyl)piperidine-4-carboxylic acid (0.035 g, 17%) as an off-white solid. ¹H NMR (400 MHz, DMSO-d₆) δ 12.26 (br, 1H), 8.42 (s, 1H), 7.88 (s, 1H), 7.83-7.81 (m, J=8.4 Hz, 1H), 7.74-7.72 (d, J=8.0 Hz, 2H), 7.45-7.43 (d, J=8.0 Hz, 2H), 5.49 (s, 2H), 4.44-4.11 (m, 3H), 4.11-3.69 (m, 6H), 3.69-3.51 (m, 5H), 3.20-3.09 (m, 2H), 2.95-2.80 (m, 2H), 2.80-2.69 (m, 2H), 2.05-1.80 (m, 4H), 1.80-1.65 (m, 2H), 1.69-1.51 (m, 4H), 1.50-1.35 (m, 4H), 1.34-1.10 (m, 5H), 1.00-0.80 (m, 3H). MS: [MH]⁺ 867.7.

### Synthesis of 2-((4-((S)-8-(((2S,3R)-3-((2-oxabicyclo[2.2.2]octan-4-yl)methoxy)-1-oxo-1-(4,7-diazaspiro[2.5]octan-7-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (I-112)

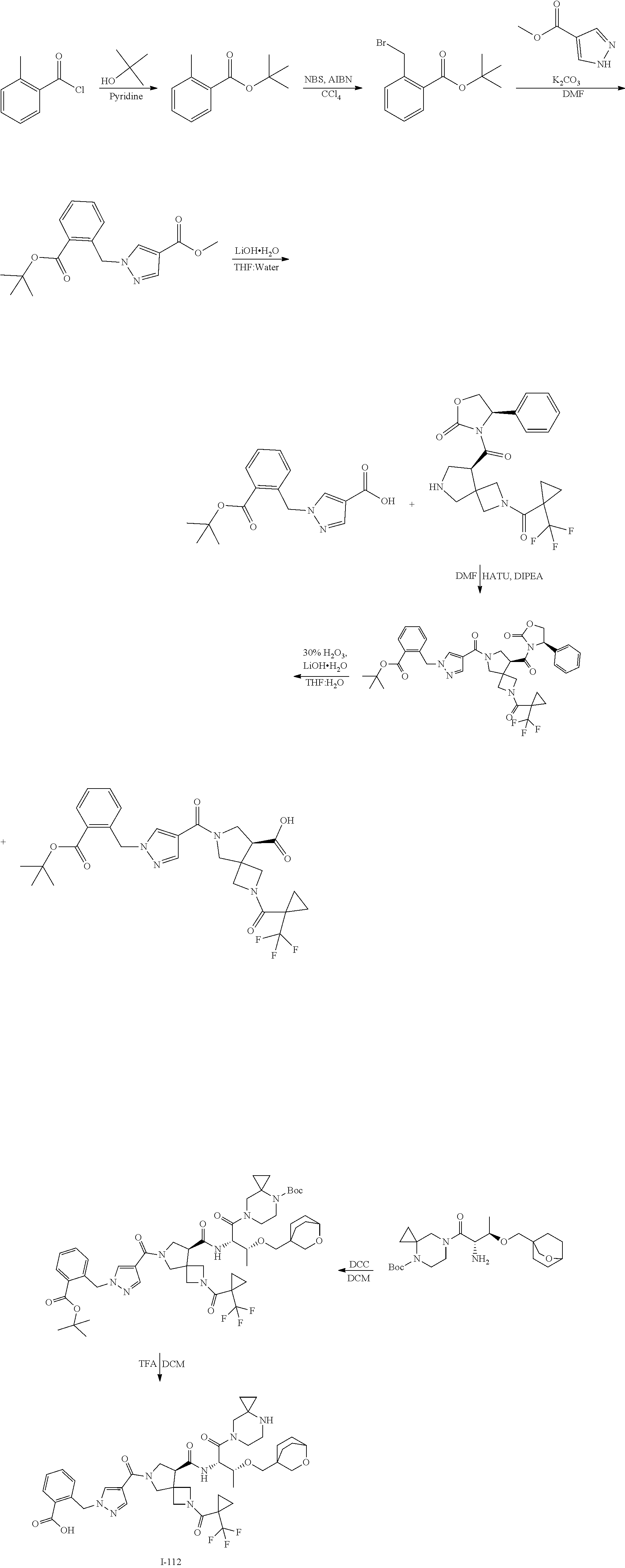

tert-Butyl 2-methylbenzoate To a stirred solution of 2-methylbenzoyl chloride (10.0 g, 64.93 mmol) in t-BuOH (14.4 g, 194.8 mmol) was added pyridine (15.3 g, 194.8 mmol) at 0° C. under nitrogen and allowed to stir at room temperature for 2 h. [Another identical batch of 10.0 g was performed and work-up together]. combined resulting reaction mixture was slowly poured into ice water (1000 mL) and was extracted with DCM (400 mL×3). Organic extracts were washed with an aqueous solution of 1N HCl, dried over anhydrous Na₂SO₄ and concentrated under reduced pressure to get a crude product. The crude product was purified by column chromatography on silica gel, using ethyl Acetate-hexane=0:1→0:1 as an eluent, to afforded tert-butyl 2-methylbenzoate (18.5 g, 74%) as a volatile oil. Product was confirmed by ¹H-NMR. ¹H-NMR (400 MHz, CDCl₃) δ 7.84-7.83 (d, J=7.2 Hz, 1H), 7.39-7.38 (t, J=1.2 Hz, 1H), 7.28-7.22 (m, 2H), 2.59 (s, 3H), 1.61 (s, 9H).

tert-Butyl 2-(bromomethyl)benzoate To a stirred solution of tert-butyl 2-methylbenzoate (18.5 g, 96.35 mmol) in CCl₄ (180 mL) was added NBS (30.8 g, 173.4 mmol) portion-wise at 0° C. under nitrogen. After 10 minutes of stirring at the same temperature, was added AIBN (1.6 g, 9.63 mmol) and stirring was continued at 80° C. for 16 h. Reaction mixture was slowly poured into ice water (1000 mL) and was extracted with DCM (300 mL×3). The organic extracts were washed with brine (200 mL), dried over anhydrous Na₂SO₄ and concentrated under reduced pressure to get a crude product, which was purified by column chromatography on silica gel, using ethyl acetate-hexane=0:10→0:10 as an eluent, to afforded tert-butyl 2-(bromomethyl)benzoate (4.5 g, 17%) as a colourless volatile oil. Product was confirmed by ¹H NMR (400 MHz, DMSO-d₆) δ 7.90-7.89 (d, J=7.2 Hz, 1H), 7.53-7.43 (m, 2H), 7.39-7.28 (m, 1H), 4.94 (s, 2H), 1.65 (s, 9H).

Methyl 1-(2-(tert-butoxycarbonyl)benzyl)-1H-pyrazole-4-carboxylate To a stirred solution of tert-butyl 2-(bromomethyl)benzoate (3.0 g, 11.11 mmol) in DMF (30 mL), was added methyl 1H-pyrazole-4-carboxylate (1.8 g, 1.33 mmol) and K₂CO₃ (3.0 g, 2.22 mmol) at room temperature and the resulting mixture stirred for 6 h at room temperature. Reaction mixture slowly poured into ice water (500 mL) and the resulting precipitate was collected by filtration. Residue was washed with cold water. Obtained solid was dried under high vacuum to get a crude product, which was purified by column chromatography on silica gel, using ethyl acetate-hexane=0:10→1.2:8.8 as an eluent, to afford methyl 1-(2-(tert-butoxycarbonyl)benzyl)-1H-pyrazole-4-carboxylate (3.0 g, 86%) as a white solid. MS: [MH]⁺ 317.2.

1-(2-(tert-butoxycarbonyl)benzyl)-1H-pyrazole-4-carboxylic acid To a stirred solution of methyl 1-(2-(tert-butoxycarbonyl)benzyl)-1H-pyrazole-4-carboxylate (3.2 g, 10.12 mmol) in a mixture of THE-water (3:1; 34 mL) was added lithium hydroxide monohydrate (1.2 g, 30.37 mmol) at room temperature under nitrogen. The resulting reaction mixture stirred at 50° C. for 6 h. Reaction mixture was concentrated under reduced pressure, obtained crude was diluted with water (100 mL) and was washed with diethyl ether (50 mL×2) to remove unwanted organic impurities. Aqueous part was acidified (pH ~3-4) with an aqueous solution of 1N HCl and was extracted with ethyl acetate (50 mL×3). The organic extracts washed with brine (50 mL), dried over anhydrous Na₂SO₄ and concentrated under reduced pressure to afford 1-(2-(tert-butoxycarbonyl)benzyl)-1H-pyrazole-4-carboxylic acid (2.6 g, 87%; crude) as a white solid. MS: [MH]⁺ 303.1.

### Synthesis of (R)-4-phenyl-3-((S)-2-(1-(trifluoromethyl)cyclopropane-1-carbonyl)-2,6-diazaspiro[3.4]octane-8-carbonyl)oxazolidin-2-one mentioned under 2-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl) piperidin-4-yl)-5-(trifluoromethyl)benzoic acid

tert-Butyl 2-((4-((S)-8-((R)-2-oxo-4-phenyloxazolidine-3-carbonyl)-2-(1-(trifluoromethyl)cyclopropane-1-carbonyl)-2,6-diazaspiro[3.4]octane-6-carbonyl)-1H-pyrazol-1-yl)methyl)benzoate To a stirred solution of 1-(2-(tert-butoxycarbonyl)benzyl)-1H-pyrazole-4-carboxylic acid (2.6 g, 8.60 mmol) in DMF (25 mL) were added DIPEA (4.4 mL, 25.82 mmol) and HATU (4.9 g, 12.91 mmol) at 0° C. under nitrogen. After 10 minutes of stirring at the same temperature, was added (R)-4-phenyl-3-((S)-2-(1-(trifluoromethyl)cyclopropane-1-carbonyl)-2,6-diazaspiro[3.4]octane-8-carbonyl)oxazolidin-2-one (4.5 g, 10.33 mmol) and stirring was continued for 1 h at room temperature. The reaction mixture slowly poured into ice water (100 mL) and the resulting precipitate was collected by filtration. Residue was washed with cold water. Obtained solid was dried under high vacuum to get a crude product. The crude product was purified by column chromatography on silica gel, using DCM-Methanol=0:10→0.5:9.5 as a gradient, to afford tert-butyl 2-((4-((S)-8-((R)-2-oxo-4-phenyloxazolidine-3-carbonyl)-2-(1-(trifluoromethyl)cyclopropane-1-carbonyl)-2,6-diazaspiro[3.4]octane-6-carbonyl)-1H-pyrazol-1-yl)methyl)benzoate (6.0 g, 97%) as a yellow solid. MS: [MH]⁺ 722.4.

(S)-6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylic acid To a stirred solution of tert-butyl 2-((4-((S)-8-((R)-2-oxo-4-phenyloxazolidine-3-carbonyl)-2-(1-(trifluoromethyl)cyclopropane-1-carbonyl)-2,6-diazaspiro[3.4]octane-6-carbonyl)-1H-pyrazol-1-yl)methyl)benzoate (5.0 g, 6.93 mmol) in THF (20 mL), was added H₂O₂ (5 mL) at 0° C. under nitrogen and allowed to stir at room temperature for 2 h. Lithium hydroxide monohydrate (0.436 g, 10.40 mmol) was added into the reaction mixture at 0° C. and stirring was continued for an additional 10 min at the same temperature. Reaction mixture was concentrated under reduced pressure, obtained crude was diluted with water (100 mL) and was washed with ethyl acetate (100 mL×2) to remove unwanted organic impurities. Aqueous part was acidified (pH ~5-6) with an aqueous solution of 1N HCl and was extracted with 20% MeOH in DCM (200 mL×3). Collected organic extracts were washed with brine (200 mL), dried over anhydrous Na₂SO₄ and concentrated under reduced pressure to afforded (S)-6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylic acid (2.7 g, 68%) as a white solid. MS: [MH]⁺ 577.3.

Tert-butyl 4-(O-((2-oxabicyclo[2.2.2]octan-4-yl)methyl)-N—((S)-6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)-2,2-dimethylpiperazine-1-carboxylate To a stirred solution of (S)-6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carboxylic acid (0.200 g, 0.34 mmol) in DCM (3 mL) were added tert-butyl 7-(O-((2-oxabicyclo[2.2.2]octan-4-yl)methyl)-L-threonyl)-4,7-diazaspiro[2.5]octane-4-carboxylate (0.300 g, 0.69 mmol) and DCC (0.140 g, 0.69 mmol) at 0° C. under nitrogen and the resulting mixture was stirred for 1 h at room temperature. [Another batch of 0.300 g was performed and worked up together]. Reaction mixture was slowly poured into ice water (50 mL) and was extracted with DCM (60 mL×3). Combined organic extracts were wash with brine (20 mL), dried over anhydrous Na₂SO₄ and concentrated under reduced pressure to get a crude product The crude product was purified by column chromatography on silica gel, using (DCM-Methanol=0:1→0.4:9.6) as an eluent, to afford tert-butyl 4-(O-((2-oxabicyclo[2.2.2]octan-4-yl)methyl)-N—((S)-6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)-2,2-dimethylpiperazine-1-carboxylate (0.700 g, 81%) as an off white solid. MS: [MH]⁺ 996.7.

2-((4-((S)-8-(((2S,3R)-3-((2-oxabicyclo[2.2.2]octan-4-yl)methoxy)-1-oxo-1-(4,7-diazaspiro[2.5]octan-7-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (I-112) To a stirred solution of tert-butyl 4-(O-((2-oxabicyclo[2.2.2]octan-4-yl)methyl)-N—((S)-6-(1-(2-(tert-butoxycarbonyl)benzyl)-1H-pyrazole-4-carbonyl)-2-(1-(trifluoromethyl)cyclopropane-1-carbonyl)-2,6-diazaspiro[3.4]octane-8-carbonyl)-L-threonyl)-2,2-dimethylpiperazine-1-carboxylate (0.700 g, 0.70 mmol) in a DCM (5 mL) was added TFA (2 mL) at 0° C. and the resulting reaction mixture stirred for 4 h at room temperature. The reaction mixture was concentrated under reduced pressure, obtained crude was diluted with water (40 mL) and basified (pH ~6-7) with an aqueous solution of NaHCO₃ and was extracted with ethyl acetate (50 mL×3) to remove unwanted impurities. Product in aqueous layer was concentrated under reduced pressure to get a crude product. Resulting crude was purified by reverse phase (C-18) silica gel column chromatography using acetonitrile-water=0:1→2:8 as gradient, to afford 2-((4-((S)-8-(((2S,3R)-3-((2-oxabicyclo[2.2.2]octan-4-yl)methoxy)-1-oxo-1-(4,7-diazaspiro[2.5]octan-7-yl)butan-2-yl)carbamoyl)-2-(1-(trifluoromethyl)cyclopropane-1-carbonyl)-2,6-diazaspiro[3.4]octane-6-carbonyl)-1H-pyrazol-1-yl)methyl)benzoic acid (0.150 g, 25%) as a white solid. ¹H-NMR (400 MHz, DMSO-d₆) δ 8.35-8.33 (br, 2H), 7.82-7.75 (m, 2H), 7.20-7.18 (m, 2H), 6.70-6.68 (m, 1H), 5.79 (s, 2H), 4.85-4.77 (m, 1H), 4.40-3.52 (m, 14H), 3.20-2.95 (m, 3H), 2.74-2.55 (m, 5H), 1.82 (s, 2H), 1.53-1.37 (m, 6H), 1.23-0.98 (m, 7H), 0.52-0.37 (m, 4H). MS: [MH]⁺ 840.5. Acid proton missing.

### Synthesis of O-((2-oxabicyclo[2.2.2]octan-4-yl)methyl)-N-(((4-nitrobenzyl)oxy)carbonyl)-L-threonine mentioned under 2-(1-(O-((2-oxabicyclo[2.2.2]octan-4-yl)methyl)-N—((S)-2-((S)-2,2-dimethylcyclopropane-1-carbonyl)-6-(5-hydroxypyrazine-2-carbonyl)-2,6-diazaspiro[3.4]octane-8-carbonyl)-L-threonyl)piperidin-4-yl)-5-(trifluoromethyl)benzoic acid

### Synthesis of tert-butyl 7-(O-((2-oxabicyclo[2.2.2]octan-4-yl)methyl)-L-threonyl)-4,7-diazaspiro[2.5]octane-4-carboxylate

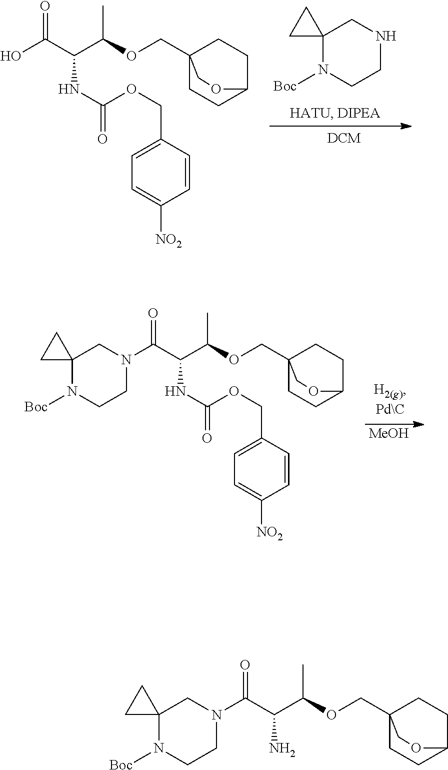

tert-Butyl 7-(O-((2-oxabicyclo[2.2.2]octan-4-yl)methyl)-N-(((4-nitrobenzyl)oxy)carbonyl)-L-threonyl)-4,7-diazaspiro[2.5]octane-4-carboxylate To a stirred solution of O-((2-oxabicyclo[2.2.2]octan-4-yl)methyl)-N-(((4-nitrobenzyl)oxy)carbonyl)-L-threonine (1.80 g, 4.26 mmol) in DCM (20 mL) were added DIPEA (1.62 g, 12.79 mmol) and HATU (3.19 g, 8.52 mmol) at 0° C. under nitrogen. After 10 minutes of stirring at the same temperature, was added tert-butyl 4,7-diazaspiro[2.5]octane-4-carboxylate (1.35 g, 4.54 mmol) and stirring was continued for 2 h at room temperature. Reaction mixture was slowly poured into ice water (100 mL) and was extracted with DCM (50 mL×3). Organic extracts washed with brine (50 mL), dried over anhydrous Na₂SO₄ and concentrated under reduced pressure to get a crude product. The crude product was purified by silica gel column chromatography, using ethyl acetate-hexane=0:1→3.5:6.5 as an eluent, to afford tert-butyl 7-(O-((2-oxabicyclo[2.2.2]octan-4-yl)methyl)-N-(((4-nitrobenzyl)oxy)carbonyl)-L-threonyl)-4,7-diazaspiro[2.5]octane-4-carboxylate (1.20 g, 46%) as a colourless oil. MS: [M-56]⁺ 561.3.

tert-Butyl 7-(O-((2-oxabicyclo[2.2.2]octan-4-yl)methyl)-L-threonyl)-4,7-diazaspiro[2.5]octane-4-carboxylate To a stirred solution of tert-butyl 7-(O-((2-oxabicyclo[2.2.2]octan-4-yl)methyl)-N-(((4-nitrobenzyl)oxy)carbonyl)-L-threonyl)-4,7-diazaspiro[2.5]octane-4-carboxylate (1.2 g, 1.94 mmol) in methanol (15 mL), was added 10% Pd on activated carbon (0.600 g) at room temperature and the resulting reaction mixture was hydrogenated under balloon pressure at room temperature for 1 h. Reaction mixture was filtered through celite bed, washed the bed with methanol (100 mL) and collected filtrates were concentrated under reduced pressure to get a crude product. The crude product was purified by silica gel column chromatography, using (MeOH-DCM=0:1-40.7:9.3) as an eluent, to afford tert-butyl 7-(O-((2-oxabicyclo[2.2.2]octan-4-yl)methyl)-L-threonyl)-4,7-diazaspiro[2.5]octane-4-carboxylate (0.800 g, 94%) as a colourless oil. MS: [MH]⁻ 438.3.

### Methyl 3-((2S,3R)-3-((2-oxabicyclo[2.2.2]octan-4-yl)methoxy)-2-((S)-6-(1-(2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butanamido)propanoate

### Synthesis of Methyl 3-((2S,3R)-3-((2-oxabicyclo[2.2.2]octan-4-yl)methoxy)-2-aminobutanamido)propanoate

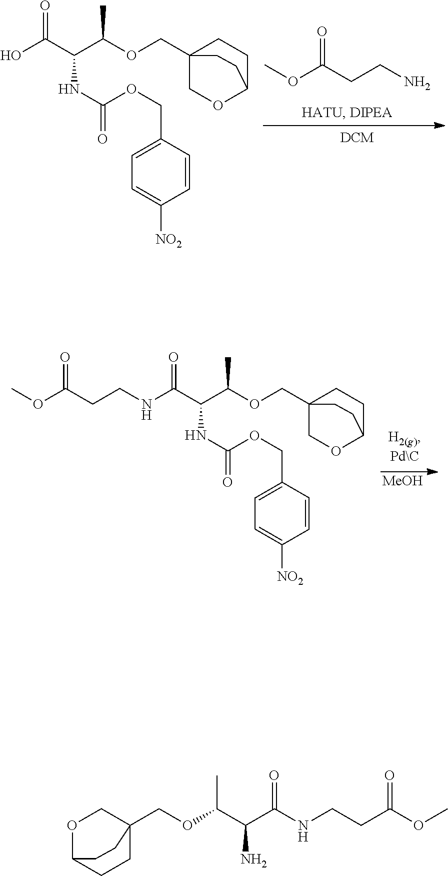

### Synthesis of O-((2-oxabicyclo[2.2.2]octan-4-yl)methyl)-N-(((4-nitrobenzyl)oxy)carbonyl)-L-threonine mentioned under 5-(1-(O-((2-oxabicyclo[2.2.2]octan-4-yl)methyl)-n-((s)-2-((s)-2,2-dimethylcyclopropane-1-carbonyl)-6-(5-hydroxypyrazine-2-carbonyl)-2,6-diazaspiro[3.4]octane-8-carbonyl)-L-threonyl) piperidin-4-yl)-2-(trifluoromethyl)benzoic acid

Methyl 4-(1-(N-(((4-nitrobenzyl)oxy)carbonyl)-O-((tetrahydro-2H-pyran-4-yl)methyl)-L-threonyl)piperidin-4-yl)benzoate To a stirred solution of O-((2-oxabicyclo[2.2.2]octan-4-yl)methyl)-N-(((4-nitrobenzyl)oxy)carbonyl)-L-threonine (2.90, 6.87 mmol) in DCM (20 mL) were added DIPEA (2.65 g, 20.61 mmol) and HATU (3.92 g, 10.30 mmol) sequentially at 0° C. under nitrogen. After 10 minutes of stirring at the same temperature, was added methyl 3-aminopropanoate (1.41 g, 13.74 mmol) and stirring was continued for 1 h at room temperature. Reaction mixture was slowly poured into ice water (200 mL) and was extracted with DCM (100 mL×3). Organic extracts washed with brine (100 mL), dried over anhydrous Na₂SO₄ and concentrated under reduced pressure to get a crude product. The crude product was purified by silica gel column chromatography, using ethyl acetate-hexane=0:1→3:2 as an eluent, to afford methyl4-(1-(N-(((4-nitrobenzyl)oxy)carbonyl)-O-((tetrahydro-2H-pyran-4-yl)methyl)-L-threonyl)piperidin-4-yl)benzoate (3.20 g, 92%) as an off-white solid. MS: [MH]⁺508.3.

Methyl 3-((2S,3R)-3-((2-oxabicyclo[2.2.2]octan-4-yl)methoxy)-2-aminobutanamido)propanoate To a stirred solution of methyl4-(1-(N-(((4-nitrobenzyl)oxy)carbonyl)-O-((tetrahydro-2H-pyran-4-yl)methyl)-L-threonyl)piperidin-4-yl)benzoate (3.20 g, 6.31 mmol) in methanol (20 mL), was added 10% Pd on activated carbon (2.0 g) at room temperature and the resulting reaction mixture was hydrogenated under balloon pressure for 3 h at room temperature. Reaction mixture was filtered through celite bed, washed the bed with methanol (100 mL) and collected filtrates were concentrated under reduced pressure to get a crude product. The crude product was purified by silica gel column chromatography, using MeOH-DCM=0:1→1:19 as an eluent, to afford methyl 3-((2S,3R)-3-((2-oxabicyclo[2.2.2]octan-4-yl)methoxy)-2-aminobutanamido)propanoate (1.40 g, 68%) as an colourless gummy. MS: [MH]+329.3.

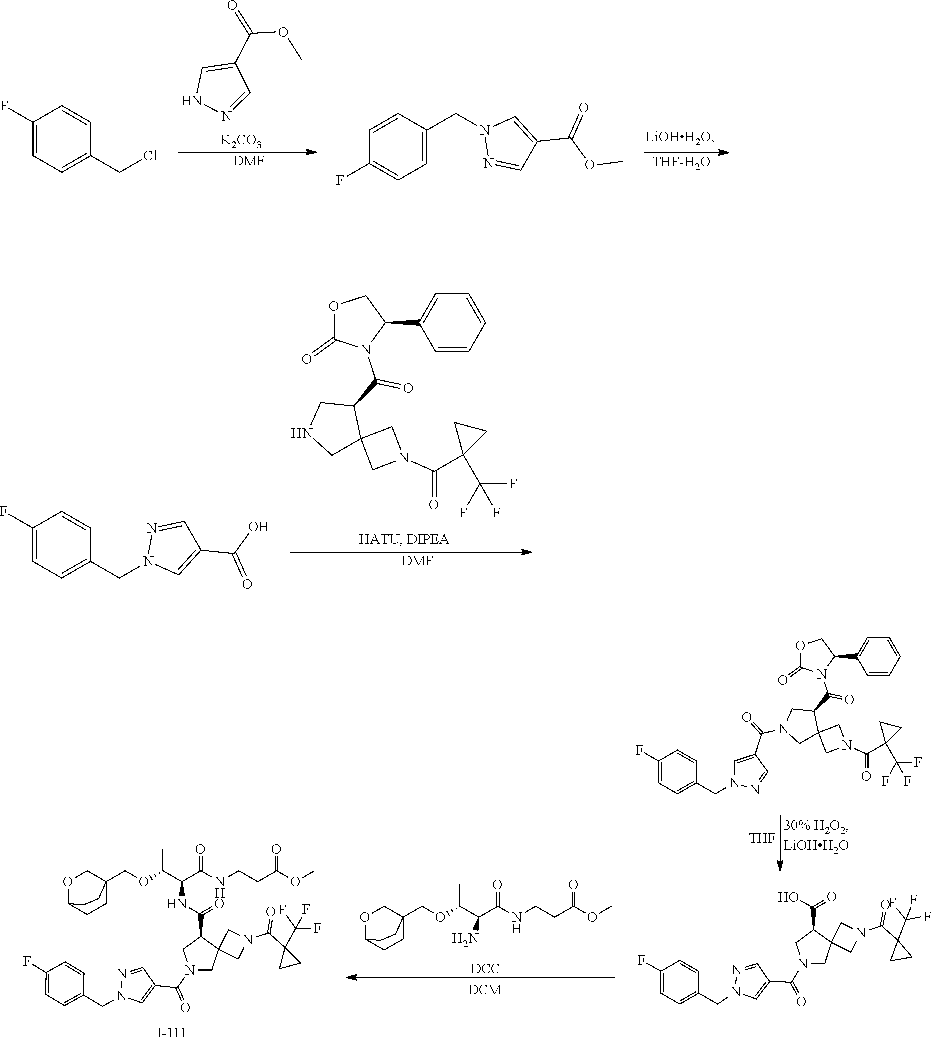

Methyl 1-(4-fluorobenzyl)-1H-pyrazole-4-carboxylate To a stirred solution of 1-(chloromethyl)-4-fluorobenzene (3.00 g, 20.80 mmol) in DMF (25 mL) were added methyl 1H-pyrazole-4-carboxylate (2.62 g, 20.80 mmol) and K₂CO₃ (6.50 g, 47.61 mmol) sequentially at room temperature and the resulting mixture was heated at 90° C. for 4 h. Reaction mixture slowly poured into ice water (200 mL) and the resulting precipitate was collected by filtration. Obtained residue was washed with cold water and dried under high vacuum to afford methyl 1-(4-fluorobenzyl)-1H-pyrazole-4-carboxylate (4.00 g, 82%; crude) as an off-white solid. The crude product was directly used for next step without further purification. MS: [MH]⁺235.1

1-(4-Fluorobenzyl)-1H-pyrazole-4-carboxylic acid To a stirred solution of methyl 1-(4-fluorobenzyl)-1H-pyrazole-4-carboxylate (4.00 g, 17.09 mmol) in a mixture of THF-water (3:1; 10 mL) was added lithium hydroxide monohydrate (2.15 g, 51.28 mmol) and MeOH (0.5 ml) at room temperature under nitrogen. The resulting reaction mixture was stirred for 2 h at room temperature. Reaction mixture was concentrated under reduced pressure, crude was taken in water (50 mL), acidified (pH ~3-4) with an aqueous solution of 1N HCl and the resulting precipitate was collected by filtration. Crude residue was washed with cold water until the pH of the filtrate became neutral (pH ~6-7). Obtained solid was triturated using diethyl ether and dried under high vacuum to afford 1-(4-fluorobenzyl)-1H-pyrazole-4-carboxylic acid (3.50 g, 93%; crude) as a white solid. MS: [MH]⁺221.1.

### Synthesis of (R)-4-phenyl-3-((S)-2-(1-(trifluoromethyl)cyclopropane-1-carbonyl)-2,6-diazaspiro[3.4]octane-8-carbonyl)oxazolidin-2-one mentioned under 2-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl) piperidin-4-yl)-5-(trifluoromethyl)benzoic acid

(R)-3-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one To a stirred solution of 1-(4-fluorobenzyl)-1H-pyrazole-4-carboxylic acid (1.00 g, 4.54 mmol) in DMF (10 mL) were added DIPEA (1.75 g, 13.63 mmol) and HATU (2.11 g, 5.55 mmol) sequentially at 0° C. under nitrogen. The resulting reaction mixture was stirred at room temperature for 30 min followed by addition of (R)-4-phenyl-3-((S)-2-(1-(trifluoromethyl)cyclopropane-1-carbonyl)-2,6-diazaspiro[3.4]octane-8-carbonyl)oxazolidin-2-one (2.38 g, 5.45 mmol) under nitrogen atmosphere. Stirring was continued for an additional 2 h at room temperature. Reaction mixture slowly poured into water (100 mL) and was extracted with ethyl acetate (50 mL×3). Collected organics were washed with brine (150 mL), dried over anhydrous Na₂SO₄ and concentrated under reduced pressure to get a crude product. The crude product was purified by silica gel column chromatography, using MeOH:DCM=0:1-41:4 as an eluent, to afforded (R)-3-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one (2.10 g, Quantitative) as a light yellow solid MS: [MH]⁺640.3.

(S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxylic acid To a stirred solution of (R)-3-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one (1.00 g, 1.56 mmol) in THF (8 mL) was added H₂O₂ (3 mL) at 0° C. and allowed to stirred at room temperature for 2 h. An aqueous (1 mL) solution of lithium hydroxide monohydrate (0.197 g, 4.69 mmol) was added at 0° C. and the resulting reaction mixture was stirred at 0° C. for another 1.5 h. Reaction mixture was diluted with water (20 mL) and was washed with diethyl ether (20 mL×2) to remove unwonted organic impurities. Aqueous solution was acidified (pH ~2-3) with an aqueous solution of 1N HCl. The product was extracted by 10% methanol in DCM (50 mL×3). Combine organic extracts were dried over Na₂SO₄ and was concentrated under reduced pressure to afford (S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxylic acid (0.490 g, 63%; crude) as an white solid. MS: [MH]⁺ 495.1.

Methyl 3-((2S,3R)-3-((2-oxabicyclo[2.2.2]octan-4-yl)methoxy)-2-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxamido)butanamido)propanoate I-111 To a stirred solution of (S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxylic acid (0.200 g, 0.404 mmol) in DCM (1.0 mL) were added methyl 3-((2S,3R)-3-((2-oxabicyclo[2.2.2]octan-4-yl)methoxy)-2-aminobutanamido)propanoate (0.265 g, 0.809 mmol) and DCC (0.166 g, 0.809 mmol) sequentially at 0° C. under nitrogen and the resulting reaction mixture stirred for 1 h at room temperature. Reaction mixture slowly poured into water (50 mL) and extracted with DCM (50 mL×3). Combined organic layers dried over anhydrous Na₂SO₄ and concentrated under reduced pressure to get a crude product. The crude product was purified by Prep HPLC using 0.1% formic acid in water and acetonitrile as gradient to afforded methyl 3-((2S,3R)-3-((2-oxabicyclo[2.2.2]octan-4-yl)methoxy)-2-((S)-6-(1-(2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butanamido)propanoate (0.060 g, 18%) as an off white solid. ¹H NMR (400 MHz, DMSO-d₆) δ 8.37-8.35 (d, J=9.6 Hz, 1H), 8.17-8.15 (d, J=8.4 Hz, 1H), 7.94 (br. s, 1H), 7.84-7.80 (d, J=15.2 Hz. 1H), 7.34-7.31 (t, J=6.4 Hz, 2H), 7.20-7.16 (t, J=8.4 Hz, 2H), 5.34 (s, 2H), 4.32-4.30 (m, 2H), 4.17-4.15 (br, 1H), 4.04-3.88 (m, 3H), 3.78-3.70 (m, 2H), 3.70-3.52 (m, 7H), 3.29-3.20 (m, 2H), 3.18-3.03 (d, J=9.2 Hz, 1H), 3.00-2.83 (m, 1H), 2.47-2.43 (m, 4H), 1.82 (brs, 2H), 1.57-1.48 (m, 4H), 1.42-1.30 (m, 2H), 1.29-1.09 (m, 4H), 1.05-0.90 (m, 3H). MS: [MH]⁺805.5.

The following compound was prepared in a manner analogous to the procedures described above Methyl 3-((2S,3R)-3-((2-oxabicyclo[2.2.2]octan-4-yl)methoxy)-2-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxamido)butanamido)propanoate I-111

### Methyl 3-((2S,3R)-3-((2-oxabicyclo[2.2.2]octan-4-yl)methoxy)-2-((S)-6-(1-(2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butanamido)propanoate (I-105)

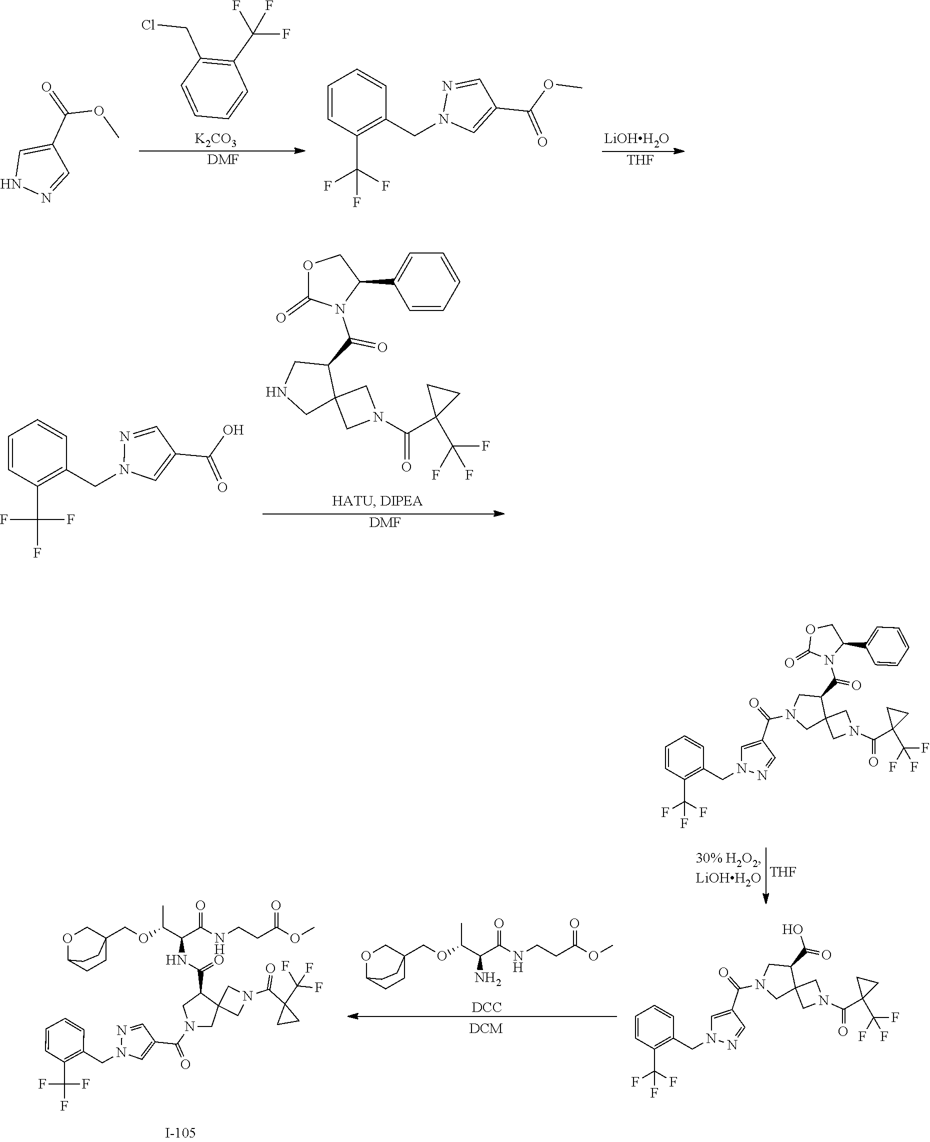

(0.050 g, 16%) as an off white solid. ¹H NMR (400 MHz, DMSO-d₆) δ 8.41-8.37 (d, J=12.8 Hz, 1H), 8.18-8.15 (d, J=8.8 Hz, 1H), 7.94-7.88 (m, 2H), 7.80-7.78 (d, J=8.0 Hz, 1H), 7.66-7.62 (t, J=7.6 Hz, 1H), 7.55-7.52 (t, J=7.6 Hz, 1H), 6.95-6.91 (t, J=8.0 Hz, 1H), 5.58 (s, 2H), 4.33-4.31 (m, 2H), 4.25-4.14 (m, 1H), 4.07-3.70 (m, 5H), 3.70-3.50 (m, 8H), 3.44-3.41 (m, 2H), 3.14-3.04 (m, 1H), 2.93-2.88 (m, 1H), 1.90-1.75 (m, 2H), 1.62-1.49 (m, 4H), 1.42-1.30 (m, 2H), 1.20-1.11 (m, 4H), 0.98-0.95 (t, J=6.0 Hz, 3H) [3 aliphatic protons are merged in DMSO-d₆] MS: [MH]⁺ 855.6.

### Methyl 3-((2S,3R)-3-((2-oxabicyclo[2.2.2]octan-4-yl)methoxy)-2-((S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butanamido)propanoate (I-110)

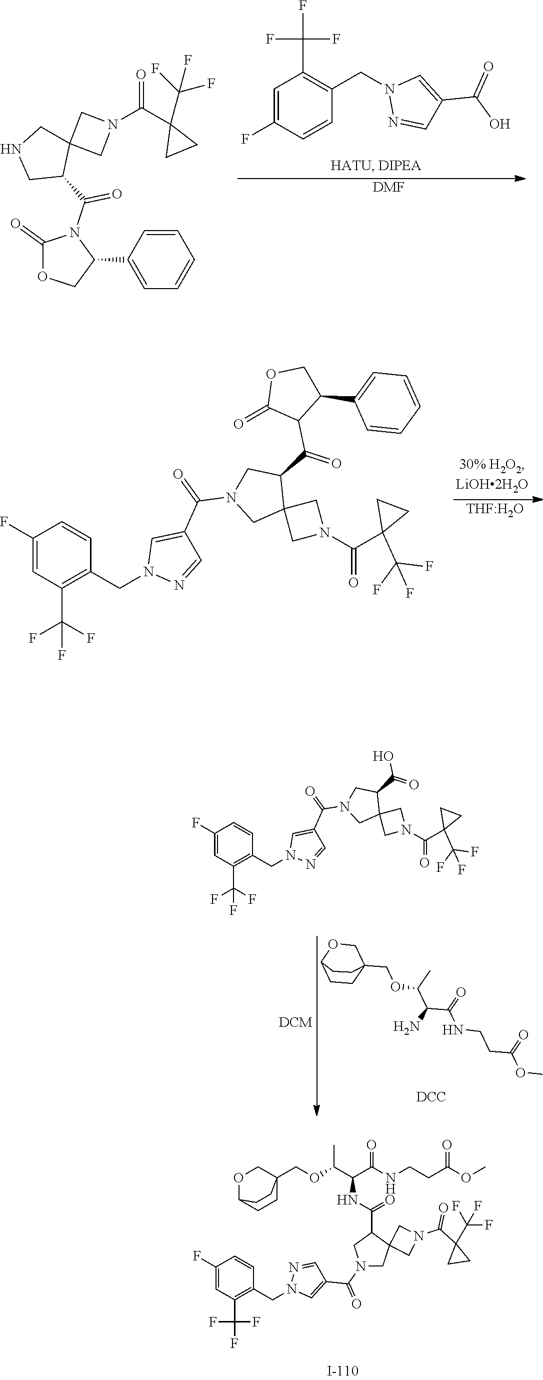

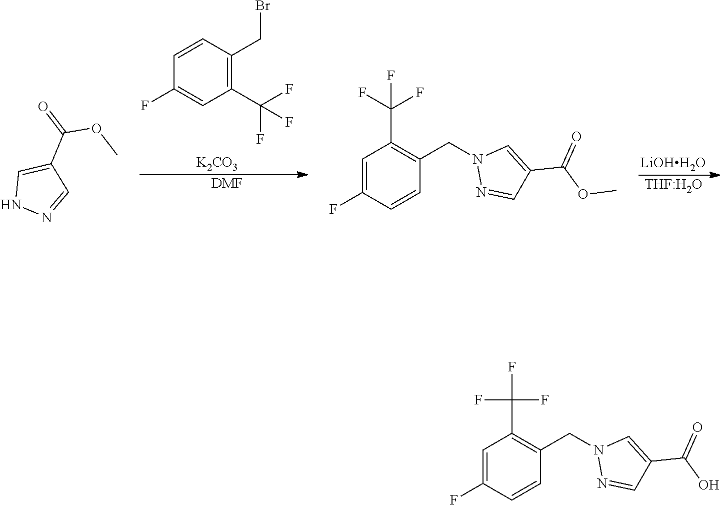

Methyl 1-(4-fluoro-2-(trifluoromethyl)benzyl)-1H-pyrazole-4-carboxylate To a stirred solution of methyl 1H-pyrazole-4-carboxylate (3.0 g, 23.78 mmol) in DMF (15 mL) was added 1-(bromomethyl)-4-fluoro-2-(trifluoromethyl)benzene (4.89 g, 19.03 mmol) and K₂CO₃ (6.56 g, 47.57 mmol) at room temperature under nitrogen. The resulting reaction mixture was heated at 90° C. temperature for 2 h. The reaction mixture slowly poured into ice water (300 mL) and the resulting precipitate was collected by filtration and residue was washed with cold water. The crude product was purified by silica gel column chromatography, using ethyl acetate:hexane=0:1→2:8 as gradient to afford methyl 1-(4-fluoro-2-(trifluoromethyl)benzyl)-1H-pyrazole-4-carboxylate (4.50 g, 62%) as a white solid. MS: [MH]⁺303.1.

1-(4-Fluoro-2-(trifluoromethyl)benzyl)-1H-pyrazole-4-carboxylic acid To a stirred solution of Methyl 1-(4-fluoro-2-(trifluoromethyl)benzyl)-1H-pyrazole-4-carboxylate (4.50 g, 14.90 mmol) in a mixture of THF-water (3:1; 10 mL) was added lithium hydroxide monohydrate (1.87 g, 44.70 mmol) at room temperature under nitrogen and the resulting mixture was stirred for 2 h at same temperature. The reaction mixture was concentrated under reduced pressure and aqueous part was acidified (pH ~3-4) with an aqueous solution of 1N HCl and the resulting precipitate was collected by filtration. Crude residue was washed with cold water until the pH of the filtrate became neutral (pH ~6-7). Obtained solid was triturated using diethyl ether and dried under high vacuum to afford (3.90 g, 91%) as a white solid. MS: [MH]⁺289.1.

### Synthetic procedure of (R)-4-phenyl-3-((S)-2-(1-(trifluoromethyl)cyclopropane-1-carbonyl)-2,6-diazaspiro[3.4]octane-8-carbonyl)oxazolidin-2-one mentioned under 2-(1-(O-((2-oxabicyclo[2.2.2]octan-4-yl)methyl)-N—((S)-6-(5-hydroxypyrazine-2-carbonyl)-2-(1-(trifluoromethyl)cyclopropane-1-carbonyl)-2,6-diazaspiro[3.4]octane-8-carbonyl)-L-threonyl) piperidin-4-yl)-5-(trifluoromethyl)benzoic acid

(R)-3-((S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one To a stirred solution of 1-(4-(trifluoromethyl)benzyl)-1H-pyrazole-4-carboxylic acid (0.900 g, 3.12 mmol) in DMF (4 mL) were added DIPEA (1.20 g, 9.37 mmol) and HATU (1.78 g, 4.68 mmol) sequentially at 0° C. under nitrogen. The resulting reaction mixture was stirred at room temperature for 30 min, followed by addition of (R)-4-phenyl-3-((S)-2-(1-(trifluoromethyl)cyclopropane-1-carbonyl)-2,6-diazaspiro[3.4]octane-8-carbonyl)oxazolidin-2-one (1.64 g, 3.750 mmol) under nitrogen atmosphere and resulting reaction mixture was stirred at same temperature for 2 h. The reaction mixture slowly poured into ice water (150 mL) and the resulting precipitate was collected by filtration and the residue was washed with cold water. The resulting crude was purified by silica gel column chromatography, using ethyl acetate:heaxane=0:1→9:1 as gradient to afford (R)-3-((S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one (1.60 g, 72%) as a white solid MS: [MH]⁺708.3.

(S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid To a stirred solution of (R)-3-((S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carbonyl)-4-phenyloxazolidin-2-one (0.500 g, 0.707 mmol) in THF (1 mL) was added H₂O₂ (1 mL) at 0° C. and stirred at room temperature for 2 h. An aqueous (1 mL) solution of lithium hydroxide monohydrate (0.044 g, 1.060 mmol) was added at 0° C. and the resulting reaction mixture was stirred at same temperature for an additional 2 h. Reaction mixture was diluted with water (50 mL) and was washed with diethyl ether (50 mL×2) to remove unwanted organic impurities. Aqueous solution was acidified (pH ~2-3) with an aqueous solution of 1N HCl and the resulting precipitate was collected by filtration. Crude residue was washed with cold water until the pH of the filtrate became neutral (pH ~6-7). Obtained solid was dried under high vacuum to afford (S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (0.300 g, 75%) as a white solid. MS: [MH]⁺ 563.2.

### Synthetic procedure of 3-((2S, 3R)-3-((2-oxabicyclo[2.2.2]octan-4-yl)methoxy)-2-aminobutanamido)propanoate mentioned under methyl 3-((2S,3R)-3-((2-oxabicyclo[2.2.2]octan-4-yl)methoxy)-2-((S)-6-(1-(4-fluorobenzyl)-1H-pyrazole-4-carbonyl)-2-(1-(trifluoromethyl)cyclopropane-1-carbonyl)-2,6-diazaspiro[3.4]octane-8-carboxamido)butanamido)propanoate

Methyl 3-((2S,3R)-3-((2-oxabicyclo[2.2.2]octan-4-yl)methoxy)-2-((S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butanamido)propanoate (I-110) To a stirred solution of (S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxylic acid (0.300 g, 0.533 mmol) in DCM (3.0 mL) were added methyl 3-((2S,3R)-3-((2-oxabicyclo[2.2.2]octan-4-yl)methoxy)-2-aminobutanamido)propanoate (0.350 g, 1.067 mmol) and DCC (0.219 g, 1.06 mmol) at 0° C. under nitrogen. The resulting reaction mixture was stirred at room temperature for 1 h. The reaction mixture slowly poured into ice water (50 mL) and extracted with DCM (50 mL×3). Combined organic layers dried over anhydrous Na₂SO₄ and concentrated under reduced pressure to get a crude product. The crude product was purified by Prep HPLC using 0.1% formic acid in water and acetonitrile:methanol:isopropyl alcohol (65:25:10) as gradient to afforded methyl 3-((2S,3R)-3-((2-oxabicyclo[2.2.2]octan-4-yl)methoxy)-2-((S)-6-(1-(4-fluoro-2-(trifluoromethyl)benzyl)-1H-pyrazole-4-carbonyl)-2-(1-(trifluoromethyl)cyclopropane-1-carbonyl)-2,6-diazaspiro[3.4]octane-8-carboxamido)butanamido)propanoate (0.055 g, 12%) as an off white solid. ¹H NMR (400 MHz, MeOH-d₄) δ 8.54 (s, 1H), 8.26-8.23 (d, J=10.8 Hz, 1H), 7.99-7-98 (d, J=5.2 Hz, 1H), 7.58-7.55 (dd, J=8.8, 2.0 Hz, 1H), 7.42-7.38 (m, 1H), 7.18-7.13 (m, 1H), 5.61 (s, 2H), 4.71-4.50 (m, 1H), 4.49-4.25 (m, 2H), 4.25-4.12 (s, 1H), 4.12-3.94 (m, 3H), 3.94-3.80 (m, 2H), 3.80-3.65 (m, 5H), 3.60-3.37 (m, 3H), 3.30-3.20 (d, J=9.2 Hz, 2H), 3.07-3.00 (m, 1H), 2.75-2.44 (m, 2H), 2.13-1.89 (m, 2H), 1.80-1.60 (m, 4H), 1.58-1.42 (m, 2H), 1.25 (s, 4H), 1.13 (s, 3H). MS: [MH]⁺ 873.6. (1 proton, probably, merged in MeOH-d₄ peak)

### Synthesis of I-113

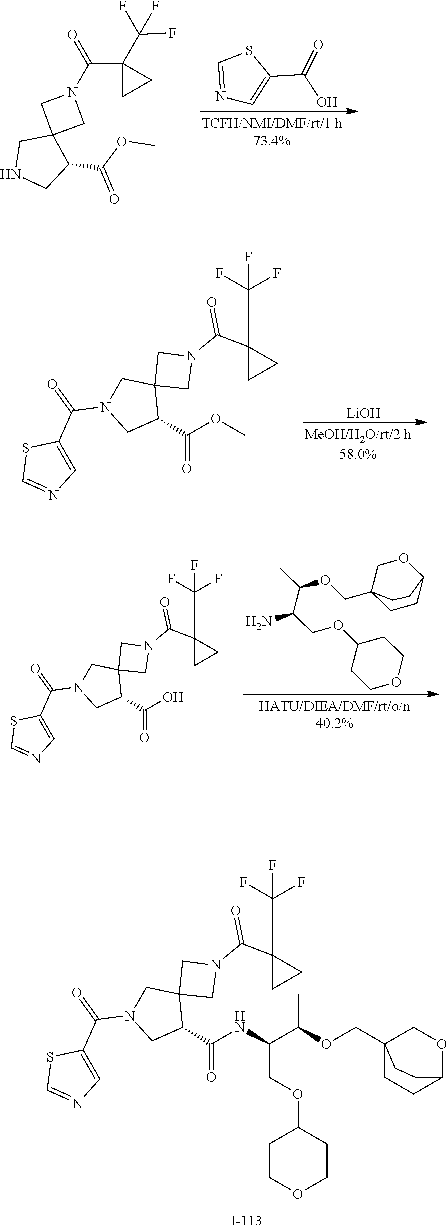

### Step 1:

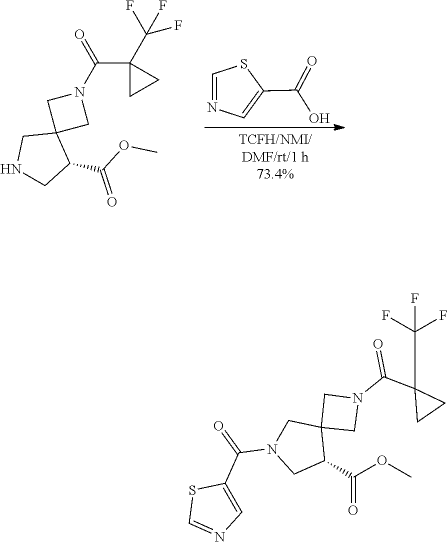 [SMILES: COC(=O)[C@@H]1CNCC12CN(C(=O)C1(C(F)(F)F)CC1)C2 ; O=C(O)c1cncs1 ; COC(=O)[C@@H]1CN(C(=O)c2cncs2)CC12CN(C(=O)C1(C(F)(F)F)CC1)C2]

A mixture of methyl (8S)-2-[1-(trifluoromethyl)cyclopropanecarbonyl]-2,6-diazaspiro[3.4]octane-8-carboxylate (0.5 g, 1.632 mmol, 1.0 equiv), 1,3-thiazole-5-carboxylic acid (0.25 g, 1.958 mmol, 1.2 equiv), TCFH (0.60 g, 2.122 mmol, 1.3 equiv) and NMI (0.47 g, 5.712 mmol, 3.5 equiv) in DMF (5 mL) were stirred for 1 h at room temperature under nitrogen atmosphere. The reaction was monitored by LCMS. The resulting mixture was quenched by addition of water and extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:1) to afford methyl (8S)-6-(1,3-thiazole-5-carbonyl)-2-[1-(trifluoromethyl)cyclopropanecarbonyl]-2,6-diazaspiro[3.4]octane-8-carboxylate (0.5 g, 73.4%) as a light yellow oil. LCMS (ESI, m/z)=418.4 [M+H]⁺.

### Step 2:

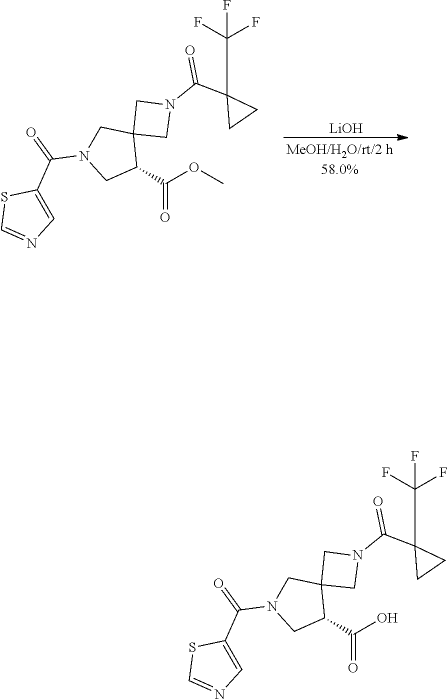 [SMILES: COC(=O)[C@@H]1CN(C(=O)c2cncs2)CC12CN(C(=O)C1(C(F)(F)F)CC1)C2 ; "LiOH" ; O=C(O)[C@@H]1CN(C(=O)c2cncs2)CC12CN(C(=O)C1(C(F)(F)F)CC1)C2]

Methyl (8S)-6-(1,3-thiazole-5-carbonyl)-2-[1-(trifluoromethyl)cyclopropanecarbonyl]-2,6-diazaspiro[3.4]octane-8-carboxylate (0.5 g, 1.198 mmol, 1.0 equiv) was dissolved in MeOH (5 mL), then a solution of LiOH (0.20 g, 4.792 mmol, 4.0 equiv) in H₂O (5 mL) was added. The resulting mixture was stirred at room temperature for 2 h. The reaction was monitored by LCMS. The reaction was concentrated under reduced pressure. The residue was purified by silica gel column chromatography, eluted with CH₂Cl₂/MeOH (5:1) to afford (8S)-6-(1,3-thiazole-5-carbonyl)-2-[1-(trifluoromethyl)cyclopropanecarbonyl]-2,6-diazaspiro[3.4]octane-8-carboxylic acid (0.28 g, 58.0%) as a white solid. LCMS (ESI, m/z)=404.0 [M+H]⁺.

### Step 3:

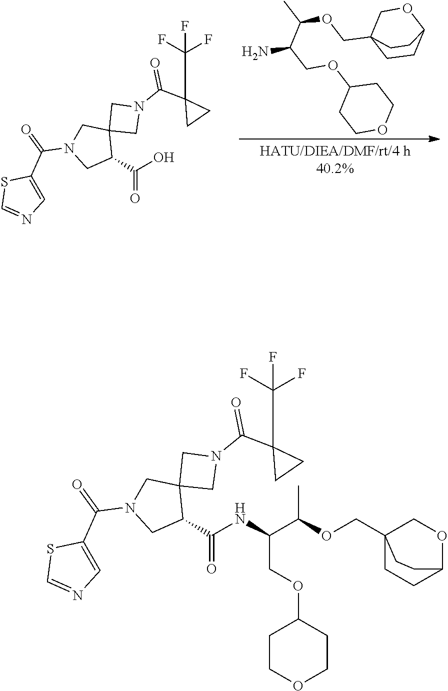 [SMILES: O=C(O)[C@@H]1CN(C(=O)c2cncs2)CC12CN(C(=O)C1(C(F)(F)F)CC1)C2 ; C[C@@H](OCC12CCC(CC1)OC2)[C@H](N)COC1CCOCC1 ; C[C@@H](OCC12CCC(CC1)OC2)[C@@H](COC1CCOCC1)NC(=O)[C@@H]1CN(C(=O)c2cncs2)CC12CN(C(=O)C1(C(F)(F)F)CC1)C2]

A mixture of (8S)-6-(1,3-thiazole-5-carbonyl)-2-[1-(trifluoromethyl)cyclopropanecarbonyl]-2,6-diazaspiro[3.4]octane-8-carboxylic acid (120 mg, 0.298 mmol, 1.0 equiv), (2R,3R)-3-{2-oxabicyclo[2.2.2]octan-4-ylmethoxy}-1-(oxan-4-yloxy)butan-2-amine (93.2 mg, 0.298 mmol, 1.0 equiv), HATU (135.7 mg, 0.357 mmol, 1.2 equiv) and DIEA (156 uL, 0.894 mmol, 3.0 equiv) in DMF (10 mL) was stirred for 4 h at room temperature under nitrogen atmosphere. The reaction was monitored by LCMS. The reaction was quenched by addition of water (100 mL), extracted with EtOAc (3×50 mL). The combined organic phase was washed with brine (1×30 mL), dried over anhydrous Na₂SO₄. After filtration, the filtrate was concentrated under reduced pressure. The crude product was purified by Prep-HPLC with the following conditions (Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 28% B to 47% B in 9 min, 47% B; Wave Length: 254/220 nm; RT1(min): 8.35) to afford (8S)—N-[(2R,3R)-3-{2-oxabicyclo[2.2.2]octan-4-ylmethoxy}-1-(oxan-4-yloxy)butan-2-yl]-6-(1,3-thiazole-5-carbonyl)-2-[1-(trifluoromethyl)cyclopropanecarbonyl]-2,6-diazaspiro[3.4]octane-8-carboxamide (83.7 mg, 40.2%) as a white solid. LCMS (ESI, m/z)=699.05 [M+H]⁺; ¹H NMR (400 MHz, Methanol-d₄) δ 9.16 (s, 1H), 8.38-8.34 (m, 1H), 4.57-4.36 (m, 2H), 4.14-3.99 (m, 5H), 3.91-3.82 (m, 4H), 3.76-3.69 (m, 3H), 3.63-3.39 (m, 7H), 3.27-3.24 (m, 1H), 3.01-2.97 (m, 1H), 1.99-1.83 (m, 4H), 1.70-1.60 (m, 4H), 1.55-1.43 (m, 4H), 1.24-1.23 (m, 4H), 1.10-1.04 (m, 3H).

### Synthesis of (R)—N-((2S,3R)-3-(cyclohexylmethoxy)-1-((S)-3-(methoxymethyl)piperidin-1-yl)-1-oxobutan-2-yl)-2-((S)-2,2-dimethylcyclopropanecarbonyl)-6-(thiazole-5-carbonyl)-2,6-diazaspiro[3.4]octane-8-carboxamide I-116

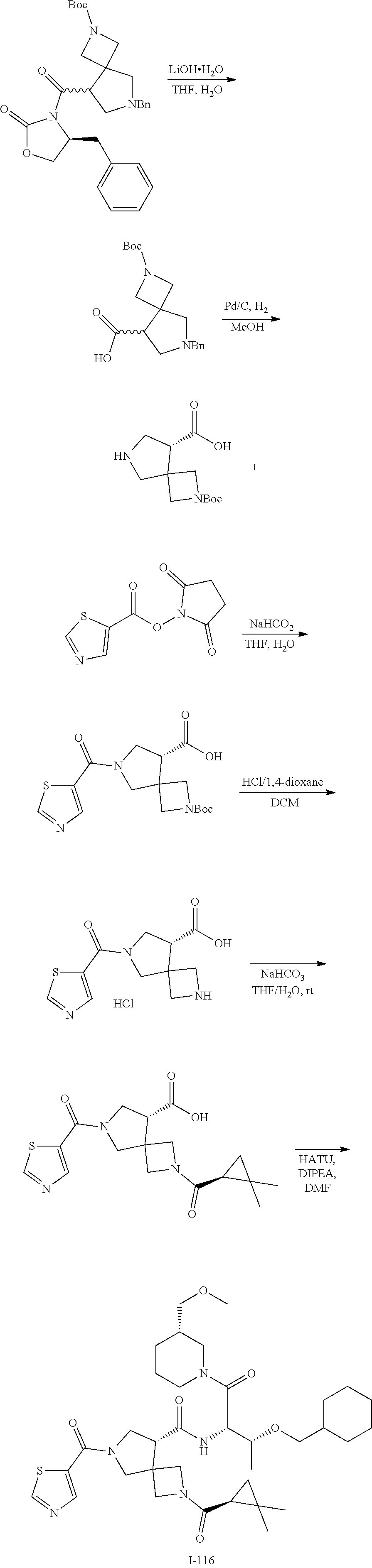

6-Benzyl-2-(tert-butoxycarbonyl)-2,6-diazaspiro[3.4]octane-8-carboxylic acid: To a solution of tert-butyl 6-benzyl-8-((S)-4-benzyl-2-oxooxazolidine-3-carbonyl)-2,6-diazaspiro[3.4]octane-2-carboxylate (9.988 g, 19.78 mmol) in tetrahydrofuran (72 mL) at 0° C. was added a solution of lithium hydroxide monohydrate (2.076 g, 49.45 mmol) and H₂O₂ (30% aqueous solution, 4.5 g, 39.56 mmol) in water (24 mL). The resulting mixture was stirred at room temperature for 1 hour. TLC showed the reaction was completed. To the reaction mixture was added a solution of sodium sulfite (5.103 g, 39.56 mmol) in water (30 mL), and the resulting mixture was extracted with MTBE (50 mL×2). The aqueous layer was acidified to pH 3-4 with hydrochloric acid (1.0N), and extracted with dichloromethane (50 mL×5). The combined organic layers were dried over anhydrous sodium sulfate, and concentrated under reduced pressure to give a crude residue which was purified by silica gel column chromatography using a 100% methanol in dichloromethane gradient to afford the title compound (2.305 g, yield 330%) as a yellow solid. ¹H NMR (400 MHz, DMSO-d₆). δ 7.64-7.62 (m, 2H), 7.45-7.44 (m, 3H), 4.34 (s, 2H), 3.95-3.79 (m, 4H), 3.53-3.39 (m, 5H), 1.37 (s, 9H).

(R)-2-(tert-butoxycarbonyl)-2,6-diazaspiro[3.4]octane-8-carboxylic acid: A mixture of 6-benzyl-2-(tert-butoxycarbonyl)-2,6-diazaspiro[3.4]octane-8-carboxylic acid (2.3 g) and palladium on carbon (10%, 1.0 g) in methanol (160 mL) was stirred under hydrogen atmosphere (hydrogen balloon) at room temperature overnight. Large amount of solid precipitate appeared. To the reaction mixture was added water (160 mL), and the mixture was stirred at room temperature for 4 hours. Palladium on carbon was removed through filtration and washed with methanol-water (1:1) (30 mL×2). The combined filtrates were concentrated under reduced pressure to afford (R)-2-(tert-butoxycarbonyl)-2,6-diazaspiro[3.4]octane-8-carboxylic acid (1.500 g, yield 88%) as an off-white solid. ¹HNMR (400 MHz, DMSO-d₆): δ 13.16 (br, 1H), 9.74 (br, 1H), 3.92-3.82 (m, 4H), 3.42-3.29 (m, 5H), 1.37 (s, 9H).

(R)-2-(tert-butoxycarbonyl)-6-(thiazole-5-carbonyl)-2,6-diazaspiro[3.4]octane-8-carboxylic acid: To a solution of (S)-2-(tert-butoxycarbonyl)-2,6-diazaspiro[3.4]octane-8-carboxylic acid (1.500 g, 5.85 mmol) in water (15 mL) was added NaHCO₃ (0.984 g, 11.71 mmol), followed by the addition of a solution of 2,5-dioxopyrrolidin-1-yl thiazole-5-carboxylate, 1.300 g, 5.85 mmol) in THE (10 mL). The resulting mixture was stirred at room temperature for 1 hours. The reaction mixture was poured into water (5 mL) and extracted with ethyl acetate (20 mL×2). The aqueous layer was acidified to pH 3-4 with hydrochloric acid (2.0 N), and extracted with 10% methanol in dichloromethane (20 mL×5). The combined organic layers were dried over anhydrous sodium sulfate and concentrated to afford (R)-2-(tert-butoxycarbonyl)-6-(thiazole-5-carbonyl)-2,6-diazaspiro[3.4]octane-8-carboxylic acid (1.5 g, yield 70%) which was used in next step without further purification. MS: [M-56]⁺ 311.9.

(R)-2-((S)-2,2-dimethylcyclopropanecarbonyl)-6-(thiazole-5-carbonyl)-2,6-diazaspiro[3.4]octane-8-carboxylic acid: To a solution of (R)-2-(tert-butoxycarbonyl)-6-(thiazole-5-carbonyl)-2,6-diazaspiro[3.4]octane-8-carboxylic acid (0.600 g, 1.63 mmol) in dichloromethane (5 mL) was added hydrogen chloride in dioxane (4.0M, 5 mL). The resulting mixture was stirred at room temperature overnight. The reaction mixture was concentrated in vacuo to give (R)-6-(thiazole-5-carbonyl)-2,6-diazaspiro[3.4]octane-8-carboxylic acid which was taken up in aqueous NaHCO₃ solution (687 mg, 8.17 mmol, in 8 mL of water), followed by the addition of a solution of (S)-2,5-dioxopyrrolidin-1-yl 2,2-dimethylcyclopropanecarboxylate, 0.345 g, 1.63 mmol) in THE (5 mL), and the resulting mixture was stirred at room temperature for 1 hours. The reaction mixture was then poured into water (5 mL) and extracted with ethyl acetate (20 mL×2). The aqueous layer was acidified to pH 3-4 with hydrochloric acid (2.0 N), and extracted with 10% methanol in dichloromethane (20 mL×5). The combined organic layers were dried over anhydrous sodium sulfate, and concentrated in vacuo to give crude (R)-2-((S)-2,2-dimethylcyclopropanecarbonyl)-6-(thiazole-5-carbonyl)-2,6-diazaspiro[3.4]octane-8-carboxylic acid (0.430 g, 72% yield over two steps) which was used in next step without further purification. MS: [MH]⁺ 364.0

(R)—N-((2S,3R)-3-(cyclohexylmethoxy)-1-((S)-3-(methoxymethyl)piperidin-1-yl)-1-oxobutan-2-yl)-2-((S)-2,2-dimethylcyclopropanecarbonyl)-6-(thiazole-5-carbonyl)-2,6-diazaspiro[3.4]octane-8-carboxamide (I-116): To a solution of (R)-2-((S)-2,2-dimethylcyclopropanecarbonyl)-6-(thiazole-5-carbonyl)-2,6-diazaspiro[3.4]octane-8-carboxylic acid (0.080 g, 0.22 mmol) in N,N-dimethylformamide (2 mL) was added (2S,3R)-2-amino-3-(cyclohexylmethoxy)-1-((S)-3-(methoxymethyl)piperidin-1-yl)butan-1-one hydrochloride (0.088 g, 0.24 mmol), N-ethyl-N-isopropylpropan-2-amine (0.2 mL, 0.66 mmol), and (2-(7-Aza-1H-benzotriazole-1-yl)-1,1,3,3-tetramethyluronium hexafluorophosphate) (0.100 g, 0.26 mmol). The resulting mixture was stirred at room temperature for 1 hour. The reaction mixture was then poured into water (10 mL) and extracted with ethyl acetate (10 mL×2). The combined organic layers were washed with brine (10 mL×2), dried over anhydrous sodium sulfate, and concentrated under reduced pressure to give a crude residue which was purified by silica gel column chromatography using a 10% methanol in dichloromethane gradient to afford (R)—N-((2S,3R)-3-(cyclohexylmethoxy)-1-((S)-3-(methoxymethyl)piperidin-1-yl)-1-oxobutan-2-yl)-2-((S)-2,2-dimethylcyclopropanecarbonyl)-6-(thiazole-5-carbonyl)-2,6-diazaspiro[3.4]octane-8-carboxamide (0.092 g, yield 62%) as a white solid. ¹HNMR (400 MHz, CD₃OD): δ 9.16 (s, 1H), 8.40-8.35 (m, 1H), 5.00-4.94 (m, 1H), 4.58-3.69 (m, 13H), 3.42-3.39 (m, 3H), 3.28-3.11 (m, 5H), 2.99-2.92 (m, 1H), 2.76-2.54 (m, 1H), 1.83-1.69 (m, 9H), 1.54-1.40 (m, 3H), 1.19-1.11 (m, 11H), 1.05-1.03 (m, 1H), 0.94-0.88 (m, 2H), 0.79-0.77 (m, 1H). MS: [MH]⁺672.3.

The following compounds of Table I-18 were prepared according to procedures described above using the appropriate commercially available reagents.

TABLE I-18

| Ex- | | | |
|---|---|---|---|
| ample | | | |
| Num- | | | |
| ber | Compound | ¹HNMR | LCMS |
| I-71 | 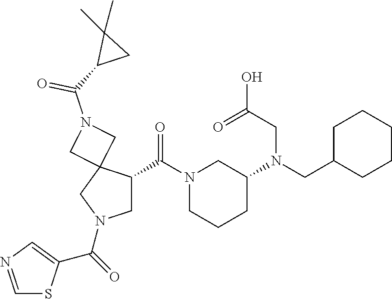 [SMILES: CC1(C)C[C@@H]1C(=O)N1CC2(CN(C(=O)c3cncs3)C[C@H]2C(=O)N2CCC[C@@H](N(CC(=O)O)CC3CCCCC3)C2)C1] | ¹H NMR (400 MHz, DMSO-d6) δ 9.16 (s, 1H), 8.12 (s, 1H), 4.71- 3.58 (m, 14H), 3.31- 3.15 (s, 1H), 3.08- 3.02 (s, 1H), 3.01-2.9 (s, 1H), 2.25-2.15 (s, 1H), 1.99-1.83 (m, 3H), 1.78-1.62 (m, 5H), 1.52-1.23 (m, 5H), 1.21-1.10 (m, 7H), 1.09-0.95 (m, 3H), 0.79 (s, 1H). | m/z = 600.3 [M + H]⁺ |
| I-62 | 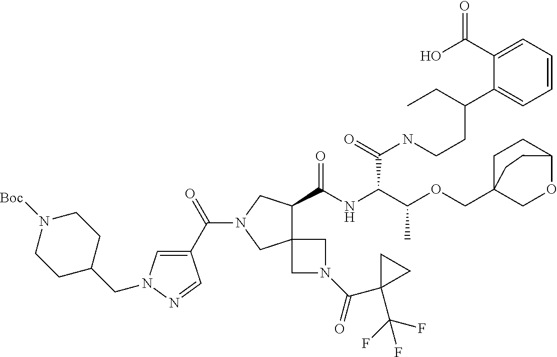 [SMILES: CCC(CCNC(=O)[C@@H](NC(=O)[C@@H]1CN(C(=O)c2cnn(CC3CCN(C(=O)OC(C)(C)C)CC3)c2)CC12CN(C(=O)C1(C(F)(F)F)CC1)C2)[C@@H](C)OCC12CCC(CC1)OC2)c1ccccc1C(=O)O] | ¹H NMR (400 MHz, Chloroform-d) δ 7.93 (d, J = 7.8 Hz, 1H), 7.81 (d, J = 9.2 Hz, 2H), 7.50 (t, J = 8.4 Hz, 1H), 7.30 (d, J = 9.6 Hz, 3H), 4.22- 3.92 (m, 12H), 3.69 (m, 5H), 3.16-3.1 (m, 2H), 2.69 (t, J = 12.8 Hz, 2H), 2.00 (d, J = 12.0 Hz, 3H), 1.61- 1.49 (m, 25H), 1.23 (s, 5H), 1.12 (s, 3H), 0.89 (s, 3H). | m/z = 996.3 [M + H]⁺ |
| I-101 | 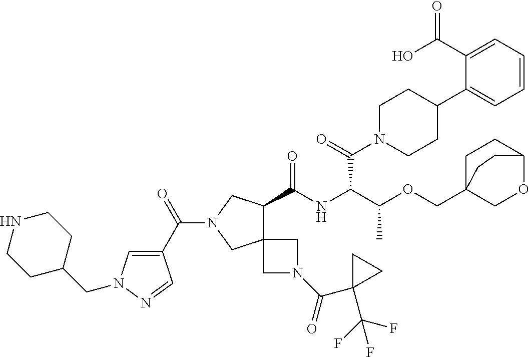 [SMILES: C[C@@H](OCC12CCC(CC1)OC2)[C@H](NC(=O)[C@@H]1CN(C(=O)c2cnn(CC3CCNCC3)c2)CC12CN(C(=O)C1(C(F)(F)F)CC1)C2)C(=O)N1CCC(c2ccccc2C(=O)O)CC1] | ¹H NMR (400 MHz, Chloroform-d) δ 7.81 (s, 2H), 7.68 (s, 1H), 7.37 (s, 3H), 7.22 (s, 1H), 5.01 (s, 1H), 4.60 (s, 1H), 4.02 (s, 12H), 3.77-3.57 (m, 7H), 3.05-2.9 (m, 6H), 3.01 (d, J = 9.4 Hz, 1H), 2.74 (s, 5H), 1.97 (s, 4H), 1.58 (s, 6H), 1.43 (s, 3H), 1.25 (s, 2H), 1.12 (s, 3H). | m/z = 896.3 [M + H]⁺ |
| I-102 | 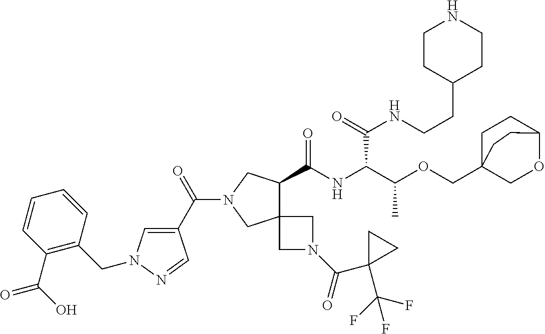 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)C1(C(F)(F)F)CC1)C2)C(=O)NCCC1CCNCC1] | ¹H NMR (400 MHz, DMSO-d6) δ 8.25 (d, J = 17.6 Hz, 1H), 8.16- 8.00 (m, 1H), 7.90- 7.71 (m, 3H), 7.49- 7.30 (m, 2H), 6.95 (d, J = 23.0 Hz, 1H), 5.78- 5.62 (m, 2H), 4.20 (s, 3H), 4.07-3.84 (m, 3H), 3.78-3.37 (m, 8H), 3.25-2.87 (m, 7H), 2.78-2.65 (m, 2H), 1.88-1.70 (m, 4H), 1.62-1.45 (m, 5H), 1.42-1.12 (m, 10H), 1.02 (q, J = 7.4 Hz, 3H). | m/z = 856.3 [M + H]⁺ |
| I-103 | 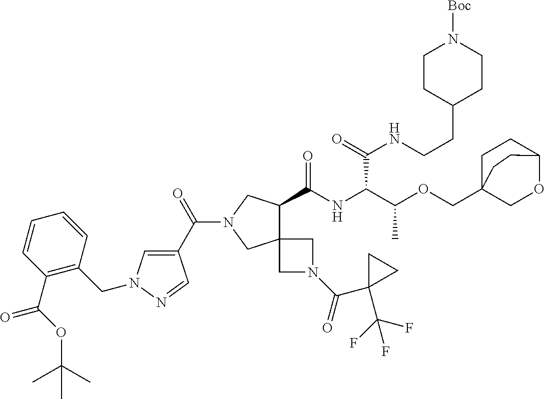 [SMILES: C[C@@H](OCC12CCC(CC1)OC2)[C@H](NC(=O)[C@@H]1CN(C(=O)c2cnn(Cc3ccccc3C(=O)OC(C)(C)C)c2)CC12CN(C(=O)C1(C(F)(F)F)CC1)C2)C(=O)NCCC1CCN(C(=O)OC(C)(C)C)CC1] | ¹H NMR (400 MHz, DMSO-d₆) δ 8.27 (d, J = 14.6 Hz, 1H), 8.09 (d, J = 10.2 Hz, 1H), 7.90- 7.82 (m, 2H), 7.75 (s, 1H), 7.51 (t, J = 7.6 Hz, 1H), 7.42 (t, J = 7.6 Hz, 1H), 6.78 (t, J = 7.4 Hz, 1H), 5.69 (d, J = 3.8 Hz, 2H), 4.40-4.09 (m, 3H), 4.03-3.84 (m, 5H), 3.80-3.37 (m, 8H), 3.20-2.89 (m, 4H), 2.65 (d, J = 15.6 Hz, 2H), 1.82 (s, 1H), 1.55 (d, J = 1.8 Hz, 15H), 1.38 (s, 15H), 1.17 (d, J = 18.2 Hz, 4H), 1.0-0.88 (m, 5H). | m/z = 1012.3 [M + H]⁺ |
| I-109 | 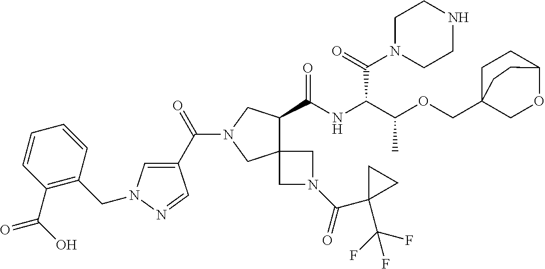 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)C1(C(F)(F)F)CC1)C2)C(=O)N1CCNCC1] | ¹H NMR (400 MHz, DMSO-d6) δ 8.62- 8.21 (m, 2H), 7.93- 7.67 (m, 2H), 7.51- 7.30 (m, 2H), 7.19-7.1 (m, 1H), 5.88-5.50 (m, 2H), 4.89-4.72 (m, 1H), 4.24-4.18 (m, 1H), 4.01-3.94 (m, 2H), 3.64-3.60 (m, 5H), 3.54-3.52 (m, 3H), 3.17 (s, 2H), 3.14 (s, 1H), 3.11 (s, 1H), 3.06-2.97 (m, 6H), 1.89-1.59 (m, 3H), 1.57-1.55 (m, 1H), 1.55-1.52 (m, | m/z = 814.5 [M + H]⁺ |
| | | 2H), 1.51-1.49 (m, | |
| | | 1H), 1.44-1.29 (m, | |
| | | 3H), 1.26-1.21 (m, | |
| | | 1H), 1.15 (s, 3H), 1.03- | |
| | | 1.00 (m, 3H). | |
| I-108 | 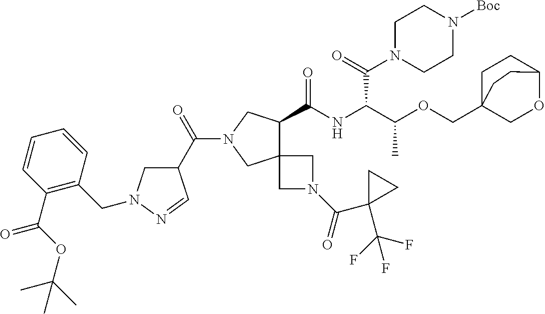 [SMILES: C[C@@H](OCC12CCC(CC1)OC2)[C@H](NC(=O)[C@@H]1CN(C(=O)C2C=NN(Cc3ccccc3C(=O)OC(C)(C)C)C2)CC12CN(C(=O)C1(C(F)(F)F)CC1)C2)C(=O)N1CCN(C(=O)OC(C)(C)C)CC1] | ¹H NMR (400 MHz, DMSO-d6) δ 8.40- 8.22 (m, 2H), 7.91- 7.80 (m, 2H), 7.57- 7.36 (m, 2H), 6.78 (t, J = 8.8 Hz, 1H), 5.73- 5.65 (m, 2H), 4.86 (s, 1H), 4.44-3.41 (m, 19H), 3.19-2.92 (m, 2H), 1.88-1.73 (m, 1H), 1.70-1.47 (m, 14H), 1.45-1.30 (m, 12H), 1.29-1.09 (m, 5H), 1.05-0.95 (m, 3H). | m/z = 970.6 [M + H]⁺ |
| I-96 | 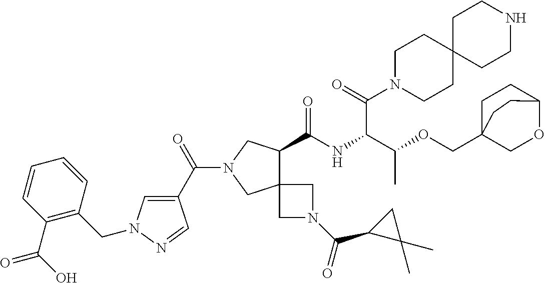 [SMILES: C[C@@H](OCC12CCC(CC1)OC2)[C@H](NC(=O)[C@@H]1CN(C(=O)c2cnn(Cc3ccccc3C(=O)O)c2)CC12CN(C(=O)[C@H]1CC1(C)C)C2)C(=O)N1CCC2(CCNCC2)CC1] | ¹H NMR (400 MHz, DMSO-d6) δ 8.68- 8.63 (m, 1H), 8.12 (d, J = 11.6 Hz, 1H), 7.78- 7.73 (m, 2H), 7.42- 7.28 (m, 3H), 6.01- 5.93 (m, 1H), 5.53- 5.45 (m, 1H), 4.62- 4.39 (m, 2H), 4.16- 3.51 (m, 16H), 3.11- 3.05 (m, 2H), 2.96- 2.91 (m, 2H), 2.75 (s, 2H), 2.75 (s, 2H), 1.82 (s, 3H), 1.54-1.39 (m, 12H), 1.11-1.05 (m, 7H), 0.99 (d, J = 6.0 Hz, 3H), 0.86-0.77 (m, 2H), 0.71-0.66 (m, 1H). | m/z = 842.6 [M + H]⁺ |
| I-114 | 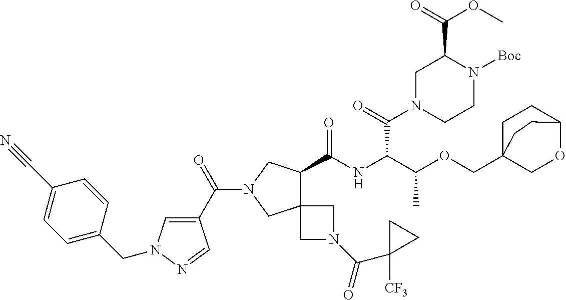 [SMILES: COC(=O)[C@@H]1CN(C(=O)[C@@H](NC(=O)[C@@H]2CN(C(=O)c3cnn(Cc4ccc(C#N)cc4)c3)CC23CN(C(=O)C2(C(F)(F)F)CC2)C3)[C@@H](C)OCC23CCC(CC2)OC3)CCN1C(=O)OC(C)(C)C] | ¹H NMR (400 MHz, CD₃OD) δ 8.31-8.23 (m, 1H), 7.97-7.91 (m, 1H), 7.72 (d, J = 8.4 Hz, 2H), 7.44- 7.37 (m, 2H), 5.48 (s, 2H), 4.97-4.85 (m, 1H), 4.76-3.91 (m, 10H), 3.88-3.33 (m, 11H), 3.22 (d, 1H), 3.16-2.91 (m, 2H), 2.02-1.55 (m, 6H), 1.54-1.36 (m, 12H), 1.27-1.21 (m, 4H), 1.13-1.07 (m, 3H). | m/z = 953.6 [M + H]⁺ |
| I-115 | 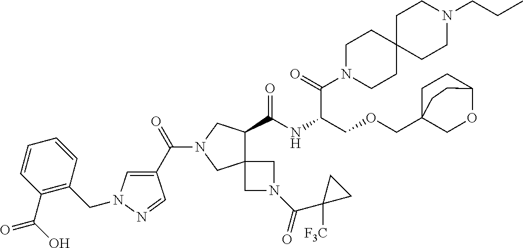 | ¹H NMR (400 MHz, Methanol-d4) δ 8.35 (s, 1H), 8.19 (d, J = 16.4 Hz, 1H), 7.85- 7.75 (m, 2H), 7.45- 7.34 (m, 2H), 7.19 (m, 1H), 6.13-5.06 (m, 2H), 4.67-3.82 (m, 8H), 3.81-3.58 (m, 6H), 3.57-3.32 (m, 3H), 3.30-3.09 (m, 5H), 3.02-2.95 (m, 4H), 2.05-1.56 (m, 12H), 1.55-1.30 (m, 5H), 1.24 (d, J = 5.2 Hz, 5H), 1.14-1.08 (m, 3H), 1.05-0.97 (m, 3H). | m/z = 922.4 [M − H]⁻ |

### Example A1: CDK2/Cyclin E1 Caliper Assay

Inhibition of CDK2/Cyclin E1 activity in the presence of compounds of the present disclosure was evaluated using a CaliperLabChip® EZ Reader mobility shift assay. In the assay, CDK2/Cyclin E1 (Eurofins, 14-475) catalyzed the phosphorylation of a fluorescently tagged peptide 5-FAM-QSPKKG-CONH2 (PerkinEliner, FL Peptide 18) which induced a difference in capillary electrophoresis mobility. The peptide substrate and product were measured, and the conversion ratio was used to determine the inhibition (as % activity and IC50 values) of CDK2/Cyclin E1. Reactions contained 50 mM HEPES pH 7.5, 10 mM MgCl2, 1 mM EDTA, 2 mM DTT, 0.01% Brij35, 0.5 mg/mL BSA, 0.1% DMSO, 2.5 nM CDK2/Cyclin E1, 100 μM ATP, and 1.5 μM fluorescent peptide substrate.

Dose titrations of inhibitors in 100% DMSO were combined with 3.25 nM CDK2/Cyclin E1 and 130 μM of ATP in reaction buffer. The mixtures were incubated for 30 minutes before the addition of fluorescent peptide substrate to initiate the kinase reaction. The final conditions were 2.5 nM CDK2/Cyclin E1, 100 μM ATP, and 1.5 μM fluorescent peptide. The reactions were stopped after 100 minutes with the addition of EDTA (400 mM final EDTA concentration). The stopped reactions were analyzed on a Caliper LabChip® EZ Reader II. The conversion ratios were normalized to yield % activity, plotted against compound concentration, and fit to a four-parameter equation to determine the IC50 for each compound.

The results of the Caliper Assay are reported in Table 3, below. Compounds with an IC₅₀ less than or equal to 0.01 μM are designated as “A”. Compounds with an IC₅₀ greater than 0.01 μM and less than or equal to 0.1 μM are designated as “B”. Compounds with an IC₅₀ greater than 0.1 μM and less than or equal to 1.0 μM are designated as “C”. Compounds with an IC₅₀ greater than 1.0 μM and less than or equal to 10.0 μM are designated as “D”. Compounds with an IC50 greater than 10.0 μM are designated as “E”.

### Example A2: ADPGLO (CDK2/E1-37C)

Inhibition of CDK2/Cyclin E1 activity by the presence of small molecules was evaluated using ADP-Glo Luminescent Kinase Assay (Promega). Activated CDK2/Cyclin E1 was incubated with its substrate Histone H1 (SignalChem H10-54N) in the kinase reaction buffer (100 μM ATP, 50 mM HEPES pH 7.5, 10 mM MgCl2, 1 mM EDTA, 2 mM DTT, 0.01% Brij35, 0.5 mg/mL BSA). Luminescence was recorded with an Envision plate reader (PerkinElmer).

Dose titrations of inhibitors in 100% DMSO were combined with 0.36 nM CDK2/Cyclin E1 in reaction buffer. The mixtures were incubated for 60 minutes at 37° C. before the addition of ATP and Histone H1 substrate to initiate the kinase reaction. The final conditions were 0.18 nM CDK2/Cyclin E1, 100 μM ATP, and 1.5 μM Histone H1. The reactions were incubated at 37° C. for 90 minutes before being stopped with the addition of ADP-Glo reagent. This mixture was incubated at room temperature for 60 minutes before Kinase Detection Solution is added to generate luminescence. The stopped reactions were analyzed on an Envision plate reader. The conversion ratios were normalized to yield % activity, plotted against compound concentration, and fit to a four-parameter equation to determine the IC₅₀ for each compound.

The results of the ADPGLO assay are reported in Table 3, below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 μM and less than or equal to 10.0 μM are designated as “C”. Compounds with an IC₅₀ greater than 10.0 μM are designated as “D”. Compounds with an IC₅₀ greater than 100.0 μM are designated as “E”.

### Example A3: IncuCyte® Cell Proliferation Assay

IncuCyte® assay was used to measure the effect of disclosed compounds on cell proliferation. Fluorescent microscopy images of cells were taken immediately after compound treatment and 72 hours later. Image analysis software was used to obtain cell counts as a function of compound concentration. Kuramochi cells labeled with mApple-H2B were seeded on 384-well assay-ready plates. Plates were placed in an IncuCyte® (Sartorius) and scanned at 0 and 72 hours. IncuCyte® software was used to count the number of fluorescent nuclei in each well. The fold change in cell count from 0 to 72 hours in wells treated with increasing compounds concentrations (10 pts, 1/2 log dilution, 20 μM top concentration) was normalized to DMSO control wells. The normalized cell counts were fit with dose response curves and a G150 was calculated.

The results of the IncuCyte® cell proliferation assay are reported in Table 3, below. Compounds with an IC₅₀ less than or equal to 0.5 μM are designated as “A”. Compounds with an IC₅₀ greater than 0.5 μM and less than or equal to 5.0 μM are designated as “B”. Compounds with an IC₅₀ greater than 5.0 μM and less than or equal to 20.0 μM are designated as “C”. Compounds with an IC₅₀ greater than 20.0 μM are designated as “D”.

TABLE 3
Assay Results

| | | | IncuCyte |
|---|---|---|---|
| | Caliper Assay | ADPGLO | Kuramochi |
| | (CDK2/CCNE1): | (CDK2/E1-37C): | RB-WT: |
| Cmpd # | IC50 (μM) | IC50 (μM) | IC50 (μM) |
| I-1 | | A | D |
| I-2 | | A | B |
| I-3 | | A | B |
| I-4 | | A | D |
| I-5 | | D | D |
| I-6 | | D | D |
| I-7 | | A | D |
| I-8 | | A | B |
| I-9 | | A | B |
| I-10 | | A | D |
| I-11 | | A | B |
| I-12 | | A | D |
| I-13 | | A | B |
| I-14 | | A | D |
| I-15 | | A | D |
| I-16 | | C | D |
| I-17 | | A | A |
| I-18 | | A | D |
| I-19 | | D | D |
| I-20 | | C | D |
| I-21 | | D | D |
| I-22 | | A | B |
| I-23 | | D | D |
| I-24 | | C | D |
| I-25 | | A | D |
| I-26 | | A | B |
| I-27 | | A | B |
| I-28 | | A | B |
| I-29 | | A | B |
| I-30 | | A | B |
| I-31 | | A | A |
| I-32 | | A | C |
| I-33 | | A | D |
| I-34 | | A | B |
| I-35 | | A | D |
| I-36 | | A | D |
| I-37 | | A | C |
| I-38 | A | A | A |
| I-39 | A | A | A |
| I-40 | A | A | D |
| I-41 | A | A | D |
| I-42 | A | A | A |
| I-43 | A | A | B |
| I-44 | E | E | D |
| I-45 | A | A | B |
| I-46 | A | A | D |
| I-47 | A | A | C |
| I-48 | | C | D |
| I-49 | C | D | D |
| I-50 | A | | B |
| I-51 | B | | B |
| I-52 | A | | B |
| I-53 | A | | A |
| I-54 | A | | B |
| I-55 | B | | D |
| I-56 | D | | D |
| I-57 | C | | D |
| I-58 | C | | D |
| I-59 | C | | C |
| I-60 | | A | B |
| I-63 | | A | D |
| I-64 1^{st} | | A | D |
| I-64 2^{nd} | | D | D |
| I-65 | | B | C |
| I-66 | | A | B |
| I-67 | | B | D |
| I-68 | | B | D |
| I-69 | | A | B |
| I-70 | | D | D |
| I-71 | | D | D |
| I-72 | | D | D |
| I-73 | | C | D |
| I-74 | | C | D |
| I-75 | | C | D |
| I-76 | | C | |
| I-77 | | D | D |
| I-78 | | A | D |
| I-79 | | A | B |
| I-80 | | B | B |
| I-81 | | A | D |
| I-82 | | | D |
| I-83 | | A | D |
| I-84 | | B | A |
| I-85 | | A | D |
| I-86 | | B | D |
| I-87 | | A | C |
| I-88 | | A | D |
| I-89 1^{st} | | D | D |
| I-89 2^{nd} | | B | D |
| I-89 3^{rd} | | A | D |
| I-90 | | A | D |
| I-91 | | A | B |
| I-92 | | A | D |
| I-93 | | A | D |
| I-94 | | A | D |
| I-95 | | D | D |
| I-96 | | A | D |
| I-97 | | D | |
| I-98 | | D | D |
| I-99 | | A | D |
| I-100 | | A | D |
| I-101 | | B | D |
| I-102 | | B | D |
| I-103 | | A | B |
| I-104 | | A | D |
| I-106 | | B | D |
| I-107 | | B | C |
| I-108 | | A | B |
| I-109 | | A | D |
| I-110 | | B | D |
| I-111 | | A | D |
| I-112 | | A | D |
| I-113 | | B | D |
| I-114 | | A | C |
| I-115 | | A | D |
| I-116 | C | | C |

1. A compound of Formula IA: 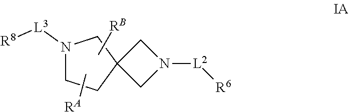
or a pharmaceutically acceptable salt thereof, wherein:
R^{A} is 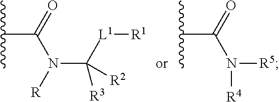
R^{B} is hydrogen, an optionally substituted C₁₋₆ aliphatic group, —OR, —NR₂ or a halogen;
L¹ is a covalent bond or a saturated or unsaturated, straight or branched, optionally substituted bivalent C₁₋₆ hydrocarbon chain, wherein 0-2 methylene units of L¹ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R¹ is hydrogen, an optionally substituted C₁₋₆ aliphatic group, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring that is optionally bridged bicyclic or spirocyclic (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur);
R² is hydrogen, an optionally substituted C₁₋₆ aliphatic group, —C₁₋₆ alkylene-OR, —C₁₋₃ alkylene-O—C₁₋₃ alkylene-R, —C(O)OR, —C(O)NR₂, —S(O)₂R, —S(O)₂NR₂, —S(O)R, —P(O)R₂, —C(O)NRS(O)₂R, or an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R³ is hydrogen; or
R² and R³ together with the intervening carbon atom form an optionally substituted 3-7 membered saturated or partially unsaturated carbocyclic ring, or an optionally substituted 3-7 membered saturated or partially unsaturated heterocyclic ring (having 1-2 heteroatoms independently selected from nitrogen, oxygen, and sulfur);
R⁴ is an optionally substituted cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur); and R⁵ is hydrogen; or
R⁴ and R⁵ together with the intervening nitrogen atom form an optionally substituted 4-7 membered saturated, or partially unsaturated heterocyclic ring (having 0-2 heteroatoms, in addition to the nitrogen, independently selected from nitrogen, oxygen, and sulfur), or an optionally substituted heteroaryl ring (having 0-3 heteroatoms, in addition to the nitrogen, independently selected from nitrogen, oxygen, and sulfur);
L² is a saturated or unsaturated, straight or branched, optionally substituted bivalent C₁₋₄ hydrocarbon chain, wherein 0-2 methylene units of L² are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁶ is an optionally substituted C₁₋₆ aliphatic group, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁷;
each instance of R⁷ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
L³ is a saturated or unsaturated, straight or branched, optionally substituted bivalent C₁₋₄ hydrocarbon chain, wherein 0-2 methylene units of L³ are independently replaced by —O—, —NR—, —S—, —OC(O)—, —C(O)O—, —C(O)—, —S(O)—, —S(O)₂—, —C(S)—, —NRS(O)₂—, —S(O)₂NR—, —NRC(O)—, —C(O)NR—, —OC(O)NR—, —NRC(O)O—, or —NRC(O)NR—;
R⁸ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted with one or more instances of R⁹;
each instance of R⁹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group, an optionally substituted C₁₋₆ aliphatic-Cy group, or Cy;
each Cy is independently an optionally substituted cyclic group selected from a 3-8 membered saturated or partially unsaturated monocyclic carbocyclic ring, phenyl, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
when there are two R groups on the same nitrogen they are taken together with their intervening atoms to form a cyclic group selected from a 4-7 membered saturated, partially unsaturated, or heteroaryl ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹;
wherein the compound is not 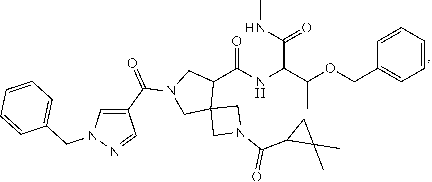 [SMILES: CNC(=O)C(NC(=O)C1CN(C(=O)c2cnn(Cc3ccccc3)c2)CC12CN(C(=O)C1CC1(C)C)C2)C(C)OCc1ccccc1]
or a pharmaceutically acceptable salt thereof.

2. (canceled)

3. The compound of claim 1, wherein R^{A} is 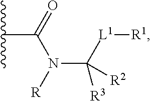
or a pharmaceutically acceptable salt thereof.

4-5. (canceled)

6. The compound of claim 1, or a pharmaceutically acceptable salt thereof, wherein R^{A} is a substituent of Table B.

7. The compound of claim 1, or a pharmaceutically acceptable salt thereof, wherein R^{B} is H.

8. The compound of claim 1, or a pharmaceutically acceptable salt thereof, wherein L¹ is

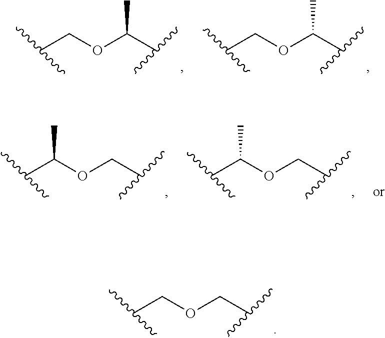

9. The compound of claim 1, or a pharmaceutically acceptable salt thereof, wherein L¹ is 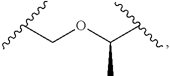 [SMILES: *CO[C@@H](*)C]
wherein the [symbol] on the left of 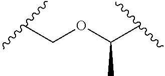 [SMILES: *CO[C@@H](*)C]
is attached to R¹.

10. The compound of claim 1, or a pharmaceutically acceptable salt thereof, wherein R¹ is

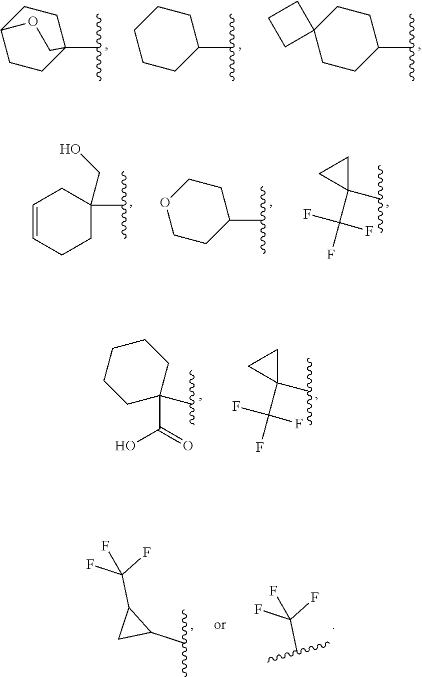

11. The compound of claim 1, or a pharmaceutically acceptable salt thereof, wherein R¹ is

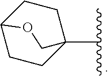 [SMILES: *C12CCC(CC1)OC2]

12. The compound of claim 1, or a pharmaceutically acceptable salt thereof, wherein R² is an optionally substituted C₁₋₆ aliphatic group, —C₁₋₆ alkylene-OR, —C(O)OR, —C(O)NR₂, —C(O)NRS(O)₂R, or an optionally substituted phenyl; and R³ is hydrogen.

13. The compound of claim 1, or a pharmaceutically acceptable salt thereof, wherein R² is —C(O)NR₂, wherein the two R groups, of —C(O)NR₂, taken together with the intervening nitrogen atom, form a cyclic group selected from a 4-7 membered saturated heterocyclic ring (having 0-3 heteroatoms, in addition to the nitrogen, independently selected from nitrogen, oxygen, and sulfur), and a 7-12 membered saturated or partially unsaturated bicyclic heterocyclic ring that is optionally bridged bicyclic or spirocyclic (having 1-4 heteroatoms, in addition to the nitrogen, independently selected from nitrogen, oxygen, and sulfur) wherein the cyclic group is optionally substituted with one or more instances of R⁹;
wherein R⁹ is selected from —CN, —C(O)O(C₁₋₃alkyl), —O(C₁₋₃alkyl), C₁₋₃haloalkyl, halo, optionally substituted C₁₋₆ aliphatic group, an optionally substituted cyclic group selected phenyl, a 7-12 membered saturated or partially unsaturated bicyclic heterocyclic ring (having 1-4 heteroatoms independently selected from nitrogen, oxygen, and sulfur), and a 5-6 membered monocyclic heteroaromatic ring (having 1-4 heteroatoms independently selected from nitrogen, oxygen, and sulfur); and
R³ is hydrogen.

14. The compound of claim 1, or a pharmaceutically acceptable salt thereof, wherein R² is a substituent of Table A.

15. The compound of claim 1, or a pharmaceutically acceptable salt thereof, wherein L² is

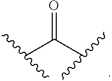 [SMILES: *C(*)=O]

16. The compound of claim 1, or a pharmaceutically acceptable salt thereof, wherein R⁶ is or

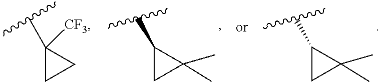

17. The compound of claim 1, or a pharmaceutically acceptable salt thereof, wherein -L²-R⁶ is a substituent of Table C.

18-48. (canceled)

49. A method of treating a disease or disorder associated with CDK2 activity in a patient comprising administering to the patient in need thereof a compound of claim 1 wherein the disease or disorder associated with CDK2 activity is a cancer selected from the group consisting of breast cancer, ovarian cancer, bladder cancer, uterine cancer, prostate cancer, lung cancer, esophageal cancer, head and neck cancer, colorectal cancer, kidney cancer, liver cancer, pancreatic cancer, stomach cancer, melanoma and thyroid cancer.

50-57. (canceled)
